SEQUENCE LISTING (1 sequence)

SEQ ID NO: 1 - protein, 470 aa, synthetic construct
  REGION 1..470: note = hPDL1(18-239)-TVMV-mIgG1(221-447)-C225S
AFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGSPGGGGGRETVRFQGGTGDAVPRDSGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2024/0409585 A1
(43) Pub. Date: Dec. 12, 2024

(54) MACROCYCLIC IMMUNOMODULATORS

(71) Applicant: Bristol-Myers Squibb Company, Princeton, NJ (US)

(72) Inventors: Martin Patrick ALLEN, Flemington, NJ (US); Claudio MAPELLI, Linden, NJ (US); Michael A. POSS, Lawrenceville, NJ (US); Yunhui ZHANG, Princeton, NJ (US); Claude QUESNELLE, Skillman, NJ (US); Tammy C. WANG, Lawrenceville, NJ (US); Jennifer X. QIAO, Princeton, NJ (US)

(21) Appl. No.: 18/290,656

(22) PCT Filed: Jul. 12, 2022
(86) PCT No.: PCT/US2022/073632

§ 371 Date: Jan. 19, 2024

Related U.S. Application Data
(60) Provisional application No. 63/223,301, filed on Jul. 19, 2021.

(51) Int. Cl. C07K 7/56; A61K 38/00

(52) U.S. Cl. CPC C07K 7/56; A61K 38/00

(57) ABSTRACT

In accordance with the present disclosure, macrocyclic compounds have been discovered that bind to PD-1 and are capable of inhibiting the interaction of PD-1 and PD-L1. These macrocyclic compounds exhibit in vitro immunomodulatory efficacy thus making them therapeutic candidates for the treatment of various diseases including cancer and infectious diseases.

### CROSS-REFERENCE TO RELATED APPLICATIONS

This application claims priority to U.S. Provisional Application No. 63/223,301, filed Jul. 19, 2021, which is herein incorporated by reference in its entirety.

### REFERENCE TO SEQUENCE LISTING SUBMITTED ELECTRONICALLY

The content of the electronically submitted sequence listing (Name: 3338_220PC01_Seqlisting; Size: 2,569 bytes; and Date of Creation: Jul. 8, 2022) is herein incorporated by reference in its entirety.

### FIELD

The present disclosure provides macrocyclic compounds that bind to PD-1 and are capable of inhibiting the interaction of PD-1 with PD-L1. These macrocyclic compounds exhibit in vitro immunomodulatory efficacy thus making them therapeutic candidates for the treatment of various diseases including cancer and infectious diseases.

### BACKGROUND

Human cancers harbor numerous genetic and epigenetic alterations, generating neoantigens potentially recognizable by the immune system (Sjoblom et al., 2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

The protein Programmed Death 1 (PD-1) is an inhibitory member of the CD28 family of receptors, that also includes CD28, CTLA-4, ICOS and BTLA. PD-1 is expressed on activated B cells, T cells, and myeloid cells (Agata et al., supra; Okazaki et al., Curr. Opin. Immunol., 14:779-782 (2002); Bennett et al., J. Immunol., 170:711-718 (2003)).

The PD-1 protein is a 55 kDa type I transmembrane protein that is part of the Ig gene superfamily (Agata et al., Int. Immunol., 8:765-772 (1996)). PD-1 contains a membrane proximal immunoreceptor tyrosine inhibitory motif (ITIM) and a membrane distal tyrosine-based switch motif (ITSM) (Thomas, M. L., J. Exp. Med, 181:1953-1956 (1995); Vivier, E. et al., Immunol. Today, 18:286-291 (1997)). Although structurally similar to CTLA-4, PD-1 lacks the MYPPY motif that is critical for CD80 CD86 (B7-2) binding. Two ligands for PD-1 have been identified, PD-L1 (B7-H1) and PD-L2 (b7-DC). The activation of T cells expressing PD-1 has been shown to be downregulated upon interaction with cells expressing PD-L1 or PD-L2 (Freeman et al., J. Exp. Med, 192:1027-1034 (2000); Latchman et al., Nat. Immunol., 2:261-268 (2001); Carter et al., Eur. J Immunol., 32:634-643 (2002)). Both PD-L1 and PD-L2 are B7 protein family members that bind to PD-1, but do not bind to other CD28 family members. The PD-L1 ligand is abundant in a variety of human cancers (Dong et al., Nat. Med, 8:787-789 (2002)). The interaction between PD-1 and PD-L1 results in a decrease in tumor infiltrating lymphocytes, a decrease in T-cell receptor mediated proliferation, and immune evasion by the cancerous cells (Dong et al., J. Mol. Med, 81:281-287 (2003); Blank et al., Cancer Immunol. Immunother., 54:307-314 (2005); Konishi et al., Clin. Cancer Res., 10:5094-5100 (2004)). Immune suppression can be reversed by inhibiting the local interaction of PD-1 with PD-L1, and the effect is additive when the interaction of PD-1 with PD-L2 is blocked as well (Iwai et al., Proc. Natl. Acad Sci. USA, 99:12293-12297 (2002); Brown et al., J. Immunol., 170:1257-1266 (2003)).

When PD-1 expressing T cells contact cells expressing its ligands, functional activities in response to antigenic stimuli, including proliferation, cytokine secretion, and cytotoxicity, are reduced. PD-1/PD-L1 or PD-L2 interactions down regulate immune responses during resolution of an infection or tumor, or during the development of self tolerance (Keir, M. E. et al., Annu. Rev. Immunol., 26:Epub (2008)). Chronic antigen stimulation, such as that which occurs during tumor disease or chronic infections, results in T cells that express elevated levels of PD-1 and are dysfunctional with respect to activity towards the chronic antigen (reviewed in Kim et al., Curr. Opin. Imm. (2010)). This is termed “T cell exhaustion”. B cells also display PD-1/PD-ligand suppression and “exhaustion”.

In addition to enhancing immunologic responses to chronic antigens, blockade of the PD-1/PD-L1 pathway has also been shown to enhance responses to vaccination, including therapeutic vaccination in the context of chronic infection (Ha, S. J. et al., “Enhancing therapeutic vaccination by blocking PD-1-mediated inhibitory signals during chronic infection”, J. Exp. Med., 205(3):543-555 (2008); Finnefrock, A. C. et al., “PD-1 blockade in rhesus macaques: impact on chronic infection and prophylactic vaccination”, J. Immunol., 182(2):980-987 (2009); Song, M.-Y. et al., “Enhancement of vaccine-induced primary and memory CD8+ t-cell responses by soluble PD-1”, J. Immunother., 34(3):297-306 (2011)).

The PD-1 pathway is a key inhibitory molecule in T cell exhaustion that arises from chronic antigen stimulation during chronic infections and tumor disease.

Accordingly, agents that block the interaction of PD-1 with PD-L1 are desired.

### SUMMARY

The present disclosure provides macrocyclic compounds which inhibit the PD-1 protein/protein interaction, and are thus useful for the amelioration of various diseases, including cancer and infectious diseases.

In a first aspect the present disclosure provides a compound of Formula (I)

or a pharmaceutically acceptable salt thereof, wherein:
R¹ is selected from C₁-C₃alkoxyC₁-C₃alkyl; C₁-C₆alkyl; C₁-C₃alkylS(O)C₁-C₆alkyl; mono-, di- or tri-C₁-C₆alkylaminoC₁-C₆alkyl; aminoC₁-C₆alkyl; aminocarbonylC₁-C₆alkyl; arylC₁-C₆alkyl; carbamidylC₁-C₆alkyl; carboxyC₁-C₃alkyl; cyanoC₁-C₆alkyl; C₃-C₆cycloalkylC₁-C₆alkyl; C₃-C₆cycloalkylcarbonylaminoC₁-C₆alkyl; heteroarylC₁-C₆alkyl; heterocyclylC₁-C₆alkyl; hydroxyC₁-C₆alkyl; H₂NC(X)NHC₁-C₆alkyl; and H₂NC(X)N C—, where X is O or NH, and N C— represents an azetidine, pyrrolidine, or piperidine ring; and wherein the aryl part of the arylC₁-C₆alkyl and the heteroaryl part of the heteroarylC₁-C₆alkyl are optionally substituted with one, two, three, four, or five groups independently selected from aminoC₂-C₆alkoxy, C₁-C₃alkyl, C₁-C₃alkylcarbonylaminoC₁-C₃alkyl, aminoC₁-C₆alkyl, R⁷⁰NHC₁-C₆alkyl, aminocarbonyl, carboxy, carboxyC₁-C₆alkoxy, carboxyC₁-C₆alkyl, guanidinylC₁-C₆alkyl, halo, haloC₁-C₃alkyl, hydroxy, nitro, and phenyl optionally substituted with a C₁-C₃alkylcarbonylamino or a carboxy group; wherein R⁷⁰ is selected from C₁-C₃alkylcarbonyl, arylC₁-C₃alkylcarbonyl, C₃-C₆cycloalkylcarbonyl, and heteroarylC₁-C₃alkylcarbonyl;
R² is selected from C₂-C₆alkenyl; C₁-C₆alkylcarbonylaminoC₁-C₆alkylthioC₁-C₆alkyl; aminocarbonylC₁-C₆alkyl; arylC₁-C₆alkyl; aryl-arylC₁-C₃alkyl; aryl-heteroarylC₁-C₃alkyl; heteroarylC₁-C₆alkyl; and hydroxyC₁-C₆alkyl; wherein the aryl part of the arylC₁-C₆alkyl and the aryl-arylC₁-C₃alkyl are optionally substituted with one, two, three, four, or five groups independently selected from C₂-C₆alkenyl, C₂-C₆alkenyloxy, C₁-C₆alkoxy, C₁-C₆alkyl, C₁-C₆alkylcarbonyloxyC₁-C₆alkoxy, C₂-C₆alkynyloxy, amino, aminoC₁-C₆alkoxy, aminoC₁-C₆alkyl, aminocarbonyl, aryloxy, carboxy, carboxyC₁-C₆alkoxy, cyano, halo, hydroxy, hydroxyC₂-C₆alkenyl, carboxyaryl, nitro, trifluoromethyl, and —OP(O)X¹X², wherein each of X¹ and X² is —OH, —NH₂, and —N(C₁-C₆alkyl)₂;
R³ is selected from aminocarbonylC₁-C₃alkyl; C₁-C₃alkylsulfonylaminocarbonylC₁-C₃alkyl; arylsulfonylaminocarbonylC₁-C₃alkyl; bis(carboxyC₁-C₃alkyl)aminoC₁-C₃alkylcarbonylaminoC₁-C₃alkyl; carboxyC₁-C₃alkyl; carboxyC₁-C₃alkylaminocarbonylC₁-C₃alkyl; carboxyC₁-C₃alkylcarbonylaminoC₁-C₃alkyl; dimethylaminosulfonylaminocarbonylC₁-C₃alkyl; heteroarylaminocarbonylC₁-C₃alkyl; (OH)₂P(O)OC₁-C₃alkyl; tetrazolylC₁-C₃alkyl; and R⁶⁵R⁶⁶C═C(CH₃)—NHC₁-C₃alkyl; wherein R⁶⁵ and R⁶⁶, together with the carbon atom to which they are attached, form a five- to seven-membered cycloalkyl ring optionally substituted with one, two, three, or four groups selected from C₁-C₃alkyl and oxo; wherein the aryl part of the arylsulfonylaminocarbonylC₁-C₃alkyl is optionally substituted with one, two, or three groups selected from C₁-C₃alkoxycarbonyl and halo;
R⁴ is selected from arylC₁-C₆alkyl and heteroarylC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, cyano, fluoroC₁-C₆alkyl, and halo;
R⁵ is selected from C₂-C₆alkenyl; C₁-C₆alkyl; C₁-C₆alkylcarbonylaminoC₁-C₆alkylthioC₁-C₆alkyl; aminoC₁-C₆alkyl; aminocarbonylC₁-C₆alkyl; arylC₁-C₆alkyl; aryl-arylC₁-C₃alkyl; arylcarbonylaminoC₁-C₃alkylarylC₁-C₃alkyl; carboxyC₁-C₆alkyl; cyanoC₁-C₆alkyl; C₃-C₈cycloalkyl; (C₃-C₈cycloalkyl)C₁-C₆alkyl; (C₃-C₈cycloalkyl)carbonylaminoC₁-C₃alkylarylC₁-C₃alkyl; heteroarylC₁-C₆alkyl; heteroaryl-arylC₁-C₃alkyl, heteroarylcarbonylaminoC₁-C₃alkylarylC₁-C₃alkyl and hydroxyC₁-C₆alkyl; wherein the aryl part of the arylC₁-C₆alkyl, the aryl-arylC₁-C₃alkyl, and the arylcarbonylaminoC₁-C₃alkylarylC₁-C₃alkyl, and the heteroaryl part of the heteroarylC₁-C₆alkyl and the heteroaryl-arylC₁-C₃alkyl are optionally substituted with one, two, three, four, or five groups independently selected from C₁-C₆alkyl, C₁-C₃alkylcarbonylamino, amino, aminoC₁-C₆alkyl, aminocarbonyl, C₁-C₃alkylaminosulfonyl, carboxy, carboxyC₁-C₆alkoxy, cyano, C₃-C₈cycloalkyl, (C₃-C₈cycloalkyl)oxy, fluoroC₁-C₆alkyl, halo, haloC₁-C₃alkyl, hydroxy, heterocyclylsulfonyl, and phenylcarbonyl;
R⁶ is aryl-arylC₁-C₃alkyl, heteroaryl-arylC₁-C₃alkyl, aryl-heteroarylC₁-C₃alkyl, heteroaryl-heteroarylC₁-C₃alkyl, wherein the aryl or the heteroaryl part is optionally substituted with one, two, three, four, or five groups independently selected from C₁-C₆alkylcarbonylamino, aminocarbonyl, fluoroC₁-C₆alkyl, halo, hydroxy, trifluoromethoxy, C₁-C₆alkoxy, C₁-C₆alkoxyC₁-C₆alkyl, carboxyC₁-C₆alkoxyC₁-C₆alkyl, cyanoC₁-C₆alkyl, and arylC₁-C₆alkoxy;
R⁷ is selected from hydrogen; C₂-C₆alkenyl; C₁-C₆alkyl; C₁-C₃alkylcarbonylaminoC₁-C₆alkyl; aminoC₁-C₆alkyl; aminocarbonylC₁-C₆alkyl; carboxyC₁-C₆alkyl; arylC₁-C₆alkyl; aryl-arylC₁-C₃alkyl; C₁-C₆alkylcarbonylaminoC₁-C₆alkylthioC₁-C₆alkyl; C₁-C₆alkylcarbonylaminoC₁-C₆alkyl; guanidinyl; heteroarylC₁-C₆alkyl; hydroxyC₁-C₆alkyl; and NH₂C(X)NHC₁-C₆alkyl, where X is O or NH; and wherein the aryl part of the arylC₁-C₆alkyl and the aryl-arylC₁-C₃alkyl, and the heteroaryl part of the heteroarylC₁-C₆alkyl are optionally substituted with one, two, three, four, or five groups independently selected from aminoC₁-C₆alkyl, aminocarbonyl, carboxy, carboxyC₁-C₆alkoxy, and hydroxy;
R⁸ is selected from C₁-C₆alkyl; aminoC₁-C₆alkyl; carboxyC₁-C₆alkyl; aryl; arylC₁-C₆alkyl; heteroarylC₁-C₆alkyl; and hydroxyC₁-C₆alkyl; wherein the aryl part of the arylC₁-C₆alkyl is optionally substituted with one, two, three, four, or five groups independently selected from halo and hydroxy;
R⁹ is selected from hydrogen; C₁-C₆alkyl; aminoC₁-C₆alkyl; aminocarbonylC₁-C₆alkyl; aryl; arylC₁-C₆alkyl; carboxyC₁-C₆alkyl; C₃-C₈cycloalkyl; C₃-C₈cycloalkylC₁-C₆alkyl; heteroarylC₁-C₆alkyl; hydroxyC₁-C₆alkyl; C₁-C₆alkylthioC₁-C₆alkyl; and NH₂C(X)NHC₁-C₆alkyl, where X is O or NH; and wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amino, carboxyC₁-C₆alkyl, cyano, halo, hydroxy, nitro, and trifluoromethyl;
R¹⁰ is selected from C₁-C₆alkyl; C₁-C₆alkylcarbonylaminoC₁-C₆alkyl; C₁-C₆alkylNHC₁-C₆alkyl; aminoC₁-C₆alkyl; aminocarbonylC₁-C₆alkyl; carboxyC₁-C₆alkyl; hydroxyC₁-C₆alkyl; NH₂C(X)NHC₁-C₆alkyl, where X is O or NH; heteroarylC₁-C₆alkyl; and arylC₁-C₆alkyl; and wherein the aryl part of the arylC₁-C₆alkyl is optionally substituted with one, two, three, four, or five aminoC₁-C₆alkyl groups;
R¹¹ is selected from C₁-C₆alky; aminoC₁-C₆alkyl; arylC₁-C₆alkyl; C₃-C₈cycloalkylC₁-C₆alkyl; heteroarylC₁-C₆alkyl; and heterocyclylC₁-C₆alkyl; wherein the aryl part of the arylC₁-C₆alkyl, the heteroaryl part of the heteroarylC₁-C₆alkyl, and the heterocyclyl part of the heterocyclylC₁-C₆alkyl are optionally substituted with one, two, three, four, or five groups independently selected from C₁-C₆alkoxy, C₁-C₆alkyl, amino, aminoC₁-C₃alkyl, halo, and hydroxy;
R¹² is selected from C₂-C₆alkenyl; C₁-C₆alkyl; C₁-C₆alkylcarbonylaminoC₁-C₆alkylthioC₁-C₆alkyl; aminoC₁-C₆alkyl; arylC₁-C₆alkyl; carboxyC₁-C₆alkyl; hydroxyC₁-C₆alkyl; and NH₂C(X)NHC₁-C₆alkyl, where X is O or NH;
R¹³ is selected from C₂-C₆alkenyl; C₁-C₆alkyl; C₁-C₆alkylcarbonylaminoC₁-C₆alkyl; C₁-C₆alkylcarbonylaminoC₁-C₆alkylthioC₁-C₆alkyl; aminoC₁-C₆alkyl; aminocarbonylC₁-C₆alkyl; carboxyC₁-C₆alkyl; carboxyC₁-C₆alkylcarbonylaminoC₁-C₃alkyl; cyanoC₁-C₆alkyl; C₃-C₈cycloalkyl; heteroarylC₁-C₆alkyl; hydroxyC₁-C₆alkyl; haloC₁-C₆alkylcarbonylaminoC₁-C₃alkyl; hydroxyC₁-C₆alkylcarbonylaminoC₁-C₃alkyl; and NH₂C(X)NHC₁-C₆alkyl, where X is O or NH;
R¹⁴ is aminocarbonyl or —C(O)NR^{14′}CR¹⁵R^{15′}, wherein
   R^{14′} is hydrogen, or R¹⁵ and R^{14′}, together with the atoms to which they are attached, form an azetidine, morpholine, piperidine, piperazine, or pyrrolidine ring, wherein each ring is optionally substituted with an amino, aminocarbonyl, or a hydroxy group;
   R¹⁵ is selected from hydrogen; C₂-C₆alkenyl; C₁-C₆alkyl; C₁-C₆alkylcarbonylaminoC₁-C₆alkyl; C₁-C₆alkylcarbonylaminoC₁-C₆alkylthioC₁-C₆alkyl; aminoC₁-C₆alkyl; aminocarbonylC₁-C₆alkyl; carboxy; carboxyC₁-C₆alkyl; heterocyclyl; hydroxyC₁-C₆alkyl; and NH₂C(X)NHC₁-C₆alkyl, where X is O or NH;
   R^{15′} is hydrogen, or R¹⁵ and R^{15′}, together with the atoms to which they are attached, form a C₃-C₈cycloalkyl ring; and
   R^{15′} is hydrogen; —C(O)NH₂, or —(CH₂)ₙC(O)NHCHR¹⁶R^{16′}; wherein
      n is 0, 1, or 2;
      R¹⁶ is selected from hydrogen, C₂-C₆alkynyl, aminoC₁-C₆alkyl, carboxyC₁-C₆alkyl, and hydroxyC₁-C₃alkyl;
      R^{16′} is hydrogen; C₁-C₆alkyl; aminocarbonyl; carboxy; or —C(O)NHCHR¹⁷R^{17′}; wherein
      R¹⁷ is hydrogen or hydroxyC₁-C₃alkyl; and
      R^{17′} is —C(O)NH₂ or —C(O)NHCHR¹⁸R^{18′}; wherein
         R¹⁸ is aminoC₁-C₆alkyl; and
         R^{18′} is carboxy.

In some aspects, R¹ is selected from aminoC₁-C₆alkyl; aminocarbonylC₁-C₆alkyl; arylC₁-C₆alkyl; C₃-C₆cycloalkylC₁-C₆alkyl; heteroarylC₁-C₆alkyl; and hydroxyC₁-C₆alkyl; wherein the aryl part of the arylC₁-C₆alkyl is optionally substituted with one, two, or three groups independently selected from C₁-C₃alkyl, carboxyC₁-C₆alkoxy, halo, and haloC₁-C₃alkyl.

In some aspects, R² is selected from aryl-arylC₁-C₂alkyl, arylC₁-C₆alkyl and heteroarylC₁-C₆alkyl, wherein the aryl part of the aryl-arylC₁-C₂alkyl and the arylC₁-C₆alkyl are optionally substituted with one, two, or three groups independently selected from carboxy, carboxyC₁-C₆alkoxy, cyano, halo, and hydroxy.

In some aspects, R³ is aminocarbonylC₁-C₃alkyl, carboxyC₁-C₃alkyl, or tetrazolylC₁alkyl.

In some aspects, R⁴ is arylC₁-C₃alkyl or heteroarylC₁-C₃alkyl, wherein the aryl part of the arylC₁-C₃alkyl and the heteroaryl part of the heteroarylC₁-C₆alkyl are optionally substituted with one, two, three, four, or five groups independently selected from C₁-C₆alkyl and cyano.

In some aspects, R⁵ is C₁-C₆alkyl; aryl-arylC₁-C₃alkyl; or arylC₁-C₆alkyl, wherein the aryl part of the arylC₁-C₆alkyl and the aryl-arylC₁-C₃alkyl are optionally substituted with one, two, or three groups independently selected from carboxy, carboxyC₁-C₆alkoxy, hydroxy, and methylcarbonylamino.

In some aspects, R⁶ is aryl-arylC₁-C₆alkyl.

In some aspects, R⁷ is selected from C₁-C₆alkyl; and arylC₁-C₆alkyl; carboxyC₁-C₆alkyl; and NH₂C(X)NHC₁-C₆alkyl, where X is O or NH; and wherein the aryl part of the arylC₁-C₆alkyl is optionally substituted with one, two, or three groups independently selected from carboxy, carboxyC₁-C₆alkoxy and hydroxy.

In some aspects, R⁸ is C₁-C₆alkyl.

In some aspects, R⁹ is C₁-C₆alkyl or arylC₁-C₆alkyl; and R⁹ is hydrogen or methyl.

In some aspects, R¹⁰ is aminoC₁-C₆alkyl; aminocarbonylC₁-C₆alkyl; or NH₂C(X)NHC₁-C₆alkyl, where X is O or NH.

In some aspects, R¹¹ is C₁-C₄alkyl or C₃-C₆cycloalkylC₁-C₃alkyl.

In some aspects, R¹² is C₁-C₄alkyl or hydroxyC₁-C₄alkyl.

In some aspects, R¹³ is aminoC₁-C₆alkyl, carboxyC₁-C₆alkyl, or hydroxyC₁-C₄alkyl.

In some aspects, R¹⁴ is aminocarbonyl or —C(O)NHCHR¹⁵C(O)NH₂; and wherein R¹⁵ is hydrogen or C₁-C₆alkyl.

In some aspects, R¹⁵ is hydrogen; C₁-C₆alkyl; aminoC₁-C₆alkyl; or carboxyC₁-C₆alkyl.

In some aspects, R¹⁶ is hydrogen or C₂-C₄alkynyl.

In some aspects, the present disclosure provides a compound of formula (I), or a pharmaceutically acceptable salt thereof, wherein
R¹ is selected from aminoC₁-C₄alkyl; aminocarbonylC₁-C₃alkyl; butyl; carbamidylC₃-C₄alkyl; cyanoC₁-C₆alkyl; cyclohexylmethyl; cyclopropylcarbonylaminopropyl; guanidinylC₃-C₄alkyl; heteroarylC₁-C₂alkyl; heterocyclylmethyl; hydroxyethyl; mono-, di-, or tri-methylaminoC₁-C₆alkyl; and H₂NC(X) , where X is O or NH, and represents a piperidine ring; arylC₁-C₂alkyl; wherein the aryl part of the arylC₁-C₂alkyl is optionally substituted with one, two, or three groups independently selected from C₁-C₃alkyl, aminocarbonyl, aminoethoxy, aminomethyl, carboxy, carboxymethoxy, halo, haloC₁-C₃alkyl, hydroxy, and nitro;
R² is selected from aryl-arylC₁-C₂alkyl; arylC₁-C₂alkyl; hydroxyethyl; heteroarylC₁-C₂alkyl; methylcarbonylaminomethylthiomethyl; and propenyl; wherein the aryl part of the aryl-arylC₁-C₂alkyl and the arylC₁-C₂alkyl are optionally substituted with one, two, or three groups independently selected from amino, aminocarbonyl, aminoethoxy, aminomethyl, aryloxy, carboxy, carboxymethoxy, cyano, halo, hydroxy, methyl, methoxy, nitro, propenoxyl, propenyl, propynoxyl, trifluoromethyl, or —OP(O)X¹X², wherein each of X¹ and X² independently is amino, hydroxy, or mono- or di-methylamino;
R³ is selected from aminocarbonylmethyl; carboxymethyl; methyl dihydrogen phosphate; and tetrazolylmethyl;
R⁴ is selected from arylmethyl and heteroarylmethyl; and wherein the aryl part of the arylmethyl and the heteroaryl part of the heteroarylmethyl are optionally substituted with one, two, three, four, or five groups independently selected from cyano, halo, methyl, methoxy, and trifluoromethyl;
R⁵ is selected from C₃-C₄alkyl; aminocarbonylethyl; aminoethyl; arylmethyl; biphenylmethyl; carboxyethyl; cyanomethyl; cyclohexylmethyl; cyclopentyl; heteroarylmethyl; hydroxypropyl; methylcarbonylaminomethylthiomethyl; and propenyl; and wherein the distal phenyl of the biphenylmethyl and the aryl part of the arylmethyl are optionally substituted with one, two, or three groups independently selected from aminocarbonyl, aminomethyl, carboxy, carboxymethoxy, halo, hydroxy, methyl, and methylcarbonylamino;
R⁶ is aryl-arylmethyl, wherein the terminal aryl part of the aryl-arylmethyl is optionally substituted with one, two, or three groups independently selected from C₁-C₂alkoxy, aminocarbonyl, benzyloxy, carboxymethoxyC₁-C₂alkyl, cyanoethyl, halo, hydroxy, methoxymethyl, methylcarbonylaminotrifluoromethoxy, heteroaryl, and, trifluoromethyl;
R⁷ is selected from hydrogen; C₁-C₅alkyl; aminoC₃-C₄alkyl; aminocarbonylC₁-C₂alkyl; arylmethyl; carboxyC₁-C₃alkyl; heteroarylmethyl; hydroxyC₁-C₃alkyl; methylcarbonylaminomethylthiomethyl; methylcarbonylaminoC₃-C₄alkyl; propenyl; and NH₂C(X)NHpropyl, where X is O or NH; and wherein the aryl part of the arylmethyl is optionally substituted with one, two, or three groups independently selected from aminocarbonyl, aminoC₁-C₂alkyl, carboxy, carboxymethoxy, and hydroxy;
R⁸ is selected from C₁-C₄alkyl; aminopropyl; aryl; arylmethyl; carboxymethyl; heteroarylmethyl; and hydroxymethyl; wherein the aryl part of the arylmethyl is optionally substituted with one, two, three, four, or five hydroxy groups;
R⁹ is selected from hydrogen; C₁-C₄alkyl; cyclohexyl; cyclohexylmethyl; aminoC₁-C₄alkyl; aminocarbonylC₁-C₆alkyl; carboxyC₁-C₆alkyl; aryl; arylmethyl; hydroxyC₁-C₂alkyl; heteroarylmethyl; methylthioethyl; and NH₂C(X)NHpropyl, where X is O or NH; and wherein the aryl part of the arylmethyl and the heteroaryl part of the heteroarylmethyl are optionally substituted with one or more groups independently selected from halo, trifluoromethyl, nitro, amino, cyano, methyl, methoxy, and carboxymethyl;
R⁹ is hydrogen or methyl;
R¹⁰ is selected from C₁-C₃alkyl; aminoC₁-C₄alkyl; aminocarbonylmethyl; carboxyC₁-C₂alkyl; hydroxyethyl; C₁-C₄alkylcarbonylaminoethyl; methylaminoethyl; and NH₂C(X)NHpropyl, where X is O or NH; heteroarylmethyl; and arylmethyl; and wherein the aryl part of the arylmethyl is optionally substituted with one, two, or three aminomethyl;
R¹¹ is selected from C₂-C₄alkyl or C₃-C₆cycloalkylmethyl;
R¹² is selected from C₃-C₄alkyl; aminoC₁-C₄alkyl; arylmethyl; carboxyC₁-C₃alkyl; hydroxyC₂-C₃alkyl; methylcarbonylaminomethylthiomethyl; propenyl; and NH₂C(X)NHpropyl, where X is O or NH;
R¹³ is selected from aminoC₁-C₄alkyl; aminocarbonylC₁-C₂alkyl; butyl; carboxyC₁-C₂alkyl; cyanomethyl; cyclopentyl; heteroarylmethyl; hydroxyC₁-C₃alkyl; methylcarbonylaminobutyl; methylcarbonylaminomethylthiomethyl; propenyl; and NH₂C(X)NHpropyl, where X is O or NH;
R¹⁴ is aminocarbonyl or —C(O)NR^{14′}CR¹⁵R^{15′}R^{15″}, wherein
   R^{14′} is hydrogen, or R¹⁵ and R^{14′}, together with the atoms to which they are attached, form a pyrrolidine ring;
   R¹⁵ is selected from hydrogen; C₁-C₃alkyl; C₁-C₄alkylcarbonylaminoethyl; aminoC₁-C₄alkyl; aminocarbonylmethyl; carboxy; carboxyC₁-C₂alkyl; heterocyclyl; hydroxyC₁-C₃alkyl; methylcarbonylaminomethylthiomethyl; propenyl; and NH₂C(X)NHpropyl, where X is O or NH;
   R^{15′} is hydrogen or R¹⁵ and R^{15′}, together with the atoms to which they are attached, form a cyclopropyl ring; and
   R^{15″} is hydrogen; aminocarbonyl; carboxy; or —(CH₂)ₙC(O)NHCHR¹⁶R^{16′}; wherein
      n is 0 or 1;
      R¹⁶ is selected from hydrogen, C₃-C₄alkynyl, aminoC₁-C₅alkyl, and carboxyethyl; and
      R^{16′} is hydrogen; C₁-C₂alkyl; aminocarbonyl; carboxy; or —C(O)NHCHR¹⁷R¹⁷;
   wherein
      R¹⁷ is hydrogen; and
      R¹⁷ is —C(O)CHR¹⁸R^{18′}; wherein
         R¹⁸ is aminoethyl; and
R^{18′} is carboxy.

In some aspects, R¹ is selected from aminoC₁-C₄alkyl; aminocarbonylC₁-C₃alkyl; arylC₁-C₂alkyl; cyclohexylmethyl; heteroarylmethyl; and hydroxyethyl; wherein the aryl part of the arylC₁-C₂alkyl is optionally substituted with one, two, or three groups independently selected from C₁-C₃alkyl, aminocarbonyl, halo, haloC₁-C₃alkyl, hydroxy, and nitro.

In some aspects, the present disclosure provides a compound of formula (I), or a pharmaceutically acceptable salt thereof, wherein:
R¹ is selected from aminoC₁-C₄alkyl; butyl; aminocarbonylC₁-C₃alkyl; arylC₁-C₂alkyl; carbamidylC₃-C₄alkyl; cyanoC₁-C₆alkyl; cyclohexylmethyl; cyclopropylcarbonylaminopropyl; guanidinylC₃-C₄alkyl; heteroarylC₁-C₂alkyl; heterocyclylmethyl; hydroxyethyl; mono-, di-, or tri-methylaminoC₁-C₆alkyl; and H₂NC(X) , where X is O or NH, and represents a piperidine ring; wherein the aryl part of the arylC₁-C₂alkyl is optionally substituted with one, two, or three groups independently selected from C₁-C₃alkyl, halo, haloC₁-C₃alkyl, nitro, aminocarbonyl, aminomethyl, aminoethoxy, carboxy, and carboxymethoxy;
R² is selected from aryl-arylC₁-C₂alkyl; arylC₁-C₂alkyl; heteroarylC₁-C₂alkyl; hydroxyethyl; methylcarbonylaminomethylthiomethyl; and propenyl; wherein the aryl part of the aryl-arylC₁-C₂alkyl and the arylC₁-C₂alkyl are optionally substituted with one, two, or three groups independently selected from amino, aminocarbonyl, aminoethoxy, aminomethyl, carboxy, carboxymethoxy, cyano, halo, hydroxy, nitro, methoxy, methyl, propenyl, trifluoromethyl, or —OP(O)X¹X², wherein each of X¹ and X² independently is hydroxy, amino, and dimethylamino;
R³ is selected from aminocarbonylmethyl; carboxymethyl; and tetrazolylmethyl;
R⁴ is selected from arylmethyl and heteroarylmethyl; and wherein the aryl part of the arylmethyl and the heteroaryl part of the heteroarylmethyl are optionally substituted with one or more groups independently selected from bromo, chloro, cyano, methoxy, methyl, and trifluoromethyl;
R⁵ is selected from C₃-C₄alkyl; arylmethyl; biphenylmethyl; cyclopentyl; cyclohexylmethyl; hydroxypropyl; and propenyl; and wherein the distal phenyl of the biphenylmethyl and the aryl part of the arylmethyl is optionally substituted with one, two, or three groups independently selected from aminocarbonyl, carboxy, and carboxymethoxy, fluoro, hydroxy, and methylcarbonylamino;
R⁶ is aryl-arylmethyl; and wherein the terminal aryl part of the aryl-arylmethyl is optionally substituted with one, two, or three groups independently selected from chloro, fluoro, and thiophenyl;
R⁷ is selected from C₁-C₅alkyl; propenyl; aminoC₃-C₄alkyl; hydroxyC₁-C₃alkyl; aminocarbonylC₁-C₂alkyl; carboxyC₁-C₃alkyl; arylmethyl; heteroarylmethyl; methylcarbonylaminoC₃-C₄alkyl; and NH₂C(X)NHpropyl, where X is O or NH; and wherein the aryl part of the arylmethyl is optionally substituted with one, two, or three groups independently selected from hydroxy, aminocarbonyl, carboxy, aminoC₁-C₂alkyl, and carboxymethoxy;
R⁸ is selected from C₁-C₄alkyl; hydroxymethyl; phenyl; and phenylmethyl; wherein the phenyl part of the phenylmethyl is optionally substituted with one, two, or three hydroxy groups;
R⁹ is selected from hydrogen; C₁-C₄alkyl; aminocarbonylC₁-C₂alkyl; arylmethyl; cyclohexyl; cyclohexylmethyl; and heteroarylmethyl; and wherein the aryl part of the arylmethyl and the heteroaryl part of the heteroarylmethyl are optionally substituted with one, two, three, four, or five groups independently selected from carboxymethyl and cyano;
R^{9′} is hydrogen;
R¹⁰ is selected from C₁-C₄alkylcarbonylaminoethyl; aminoC₁-C₄alkyl; aminocarbonylmethyl; arylmethyl; carboxyC₁-C₂alkyl; heteroarylmethyl; hydroxyethyl; methyl; methylaminoethyl; and NH₂C(X)NHpropyl, where X is O or NH; wherein the aryl part of the arylmethyl is optionally substituted with one, two, or three aminomethyl groups;
R¹¹ is selected from butyl; cyclohexylmethyl; cyclopropylmethyl; isobutyl; and isopentyl;
R¹² is selected from C₃-C₄alkyl; aminoC₃-C₄alkyl; carboxyC₁-C₃alkylisopropyl; carboxypropyl; hydroxyC₂-C₃alkyl; imidazolylmethyl; phenylmethyl; and propenyl;
R¹³ is selected from aminoC₁-C₄alkyl; aminocarbonylC₁-C₂alkyl; carboxyC₁-C₂alkyl; cyanomethyl; hydroxyC₁-C₂alkyl; methylcarbonylaminobutyl; propenyl; and NH₂C(X)NHpropyl, where X is O or NH, and
R¹⁴ is aminocarbonyl or —C(O)NR^{14′}CR¹⁵R^{15′}R^{15″}, wherein
   R^{14′} is hydrogen, or R¹⁵ and R^{14′}, together with the atoms to which they are attached, form a pyrrolidine ring;
   R¹⁵ is selected from hydrogen; aminoC₁-C₄alkyl; aminocarbonylmethyl; butylcarbonylaminoethyl; carboxy; carboxyC₁-C₂alkyl; hydroxymethyl; methyl; propenyl; methylcarbonylaminoethyl; methylcarbonylaminomethylthiomethyl; and NH₂C(X)NHpropyl, where X is O or NH;
   R^{15′} is hydrogen or R¹⁵ and R^{15′}, together with the atoms to which they are attached, form a cyclopropyl ring; and
   R^{15″} is hydrogen; aminocarbonyl; carboxy; or —(CH₂)ₙC(O)NHCHR¹⁶R^{16′}; wherein
   R¹⁶ is selected from hydrogen; C₃-C₄alkynyl; aminoC₁-C₄alkyl; and carboxyethyl; and
   R^{16′} is hydrogen; C₁-C₂alkyl; aminocarbonyl; carboxy; or —C(O)NHCHR¹⁷R^{17′}; wherein
      n is 0 or 1;
      R¹⁷ is hydrogen; and
      R^{17′} is —C(O)CHR¹⁸R^{18′}; wherein
         R¹⁸ is aminoethyl; and
         R^{18′} is carboxy.

In some aspects, the present disclosure provides a compound of formula (I), or a pharmaceutically acceptable salt thereof, wherein:
R¹ is selected from aminoC₁-C₄alkyl; aminocarbonylmethyl; arylC₁-C₂alkyl; carbamidylC₃-C₄alkyl; cyanomethyl; cyclohexylmethyl; cyclopropylcarbonylaminopropyl; guanidinylC₃-C₄alkyl; heteroarylC₁-C₂alkyl; heterocyclylmethyl; 1-hydroxyethyl; mono-, di-, or tri-methylaminoC₁-C₆alkyl; and H₂NC(X) , where X is O or NH, and represents a piperidine ring; wherein the aryl part of the arylC₁-C₂alkyl is optionally substituted with one, two, or three groups independently selected from aminocarbonyl, aminomethyl, aminoethoxy, carboxy, carboxymethoxy, methyl, fluoro, and trifluoromethyl;
R² is selected from aryl-arylC₁-C₂alkyl, arylC₁-C₂alkyl and heteroarylC₁-C₂alkyl; wherein the aryl part of the aryl-arylC₁-C₂alkyl and the arylC₁-C₂alkyl are optionally substituted with one, two, or three groups independently selected from aminocarbonyl, aminoethoxy, aminomethyl, carboxy, carboxymethoxy, cyano, fluoro, hydroxy, methoxy, methyl, nitro, and propenoxyl;
R³ is selected from aminocarbonylmethyl; carboxymethyl; and imidazolylmethyl;
R⁴ is selected from indolylmethyl and phenylmethyl, and wherein the phenyl part of the phenylmethyl is optionally substituted with one, two, or three groups independently selected from chloro, methyl, methoxy, and trifluoromethyl;
R⁵ is selected from C₃-C₄alkyl; biphenylmethyl, hydroxypropyl; hydroxyisopropyl; and phenymethyl; and wherein the distal phenyl of the biphenylmethyl and the phenyl part of the phenylmethyl are optionally substituted with one, two, or three groups independently selected from aminocarbonyl, carboxy, carboxymethoxy, fluoro, hydroxy, and methylcarbonylamino;
R⁶ is biphenylmethyl;
R⁷ is selected from C₃-C₄alkyl; aminocarbonylC₁-C₂alkyl; aminopropyl; carboxyethyl; hydroxyC₂-C₃alkyl; imidazolylmethyl; methylcarbonylaminobutyl; phenylmethyl; and NH₂C(X)NHpropyl, where X is O or NH; and wherein the phenyl part of the phenylmethyl is optionally substituted with one, two, or three groups independently selected from aminocarbonyl, aminomethyl, carboxy, carboxymethoxy, and hydroxy;
R⁸ is selected from C₁-C₄alkyl; hydroxymethyl; and phenylmethyl; wherein the phenyl part of the phenylmethyl is optionally substituted with one or two hydroxy groups;
R⁹ is selected from isobutyl and methyl;
R⁹ is hydrogen;
R¹⁰ is selected from aminoC₁-C₄alkyl; aminocarbonylmethyl; carboxymethyl; methyl; methylcarbonylaminoethyl; and NH₂C(X)NHpropyl, where X is O or NH;
R¹¹ is selected from cyclohexylmethyl and isobutyl;
R¹² is selected from C₃-C₄alkyl; aminoC₃-C₄alkyl; hydroxyC₂-C₃alkyl; and phenylmethyl;
R¹³ is selected from aminopropyl; aminocarbonylC₁-C₂alkyl; carboxyethyl; hydroxyC₁-C₂alkyl; imidazolylmethyl; methylcarbonylaminobutyl; and NH₂C(X)NHpropyl, where X is O or NH;
R¹⁴ is aminocarbonyl or —C(O)NR^{14′}CR¹⁵R^{15′}R^{15″}, wherein
   R^{14′} is hydrogen;
   R¹⁵ is selected from hydrogen; aminoC₁-C₃alkyl; aminocarbonylmethyl; butylcarbonylaminoethyl; carboxy; carboxyC₁-C₂alkyl; hydroxymethyl; methyl; and methylcarbonylaminoethyl;
   R^{15′} is hydrogen or R¹⁵ and R^{15′}, together with the atoms to which they are attached, form a cyclopropyl ring; and
   R^{15″} is hydrogen; aminocarbonyl; carboxy; or —(CH₂)ₙC(O)NHCHR¹⁶R^{16′}; wherein
      n is 0 or 1;
      R¹⁶ is selected from hydrogen; C₃-C₄alkynyl; and aminoC₁-C₄alkyl; and
      R^{16′} is hydrogen; C₁-C₂alkyl; aminocarbonyl; or carboxy.

In some aspects, the present disclosure provides a compound of formula (I), or a pharmaceutically acceptable salt thereof, wherein:
R¹ is selected from aminocarbonylmethyl; aminoethyl; aminomethyl; aminopropyl; cyclohexylmethyl; 1-hydroxyethyl; imidazolylmethyl; morpholinylmethyl; phenylmethyl; pyridylmethyl; and thienylmethyl; wherein the phenyl part of the phenylmethyl is optionally substituted with a carboxymethoxy, methyl, halo, or trifluoromethyl group;
R² is selected from biphenylmethyl, phenylmethyl, and pyridylmethyl; wherein the distal phenyl of the biphenylmethyl, and the phenyl part of the phenylmethyl are optionally substituted with carboxy, carboxymethoxy, or hydroxy;
R³ is carboxymethyl;
R⁴ is selected from indolylmethyl and phenylmethyl, wherein the phenyl part of the phenylmethyl is optionally substituted with a methyl or a trifluoromethyl group;
R⁵ is selected from C₃-C₄alkyl, biphenylmethyl, and phenymethyl, and wherein distal phenyl of the biphenylmethyl and the phenyl part of the phenylmethyl are optionally substituted with aminocarbonyl, carboxy, carboxymethoxy, methylcarbonylamino, or fluoro;
R⁶ is biphenylmethyl;
R⁷ is selected from C₃-C₄alkyl; aminocarbonylethyl; phenylmethyl; and NH₂C(X)NHpropyl, where X is O or NH; and wherein the phenyl part of the phenylmethyl is optionally substituted with one or two groups independently selected from aminocarbonyl, carboxy, carboxymethoxy, and hydroxy;
R⁸ is methyl;
R⁹ is selected from methyl and butyl;
R^{9′} is hydrogen;
R¹⁰ is selected from aminocarbonylmethyl and aminoethyl;
R¹¹ is selected from butyl and cyclohexylmethyl;
R¹² is selected from hydroxypropyl and propyl;
R¹³ is selected from aminopropyl; carboxyethyl; hydroxyC₁-C₂alkyl; imidazolylmethyl; and methylcarbonylaminobutyl;
R¹⁴ is aminocarbonyl or —C(O)NR^{14′}CR¹⁵R^{15′}R^{15″}, wherein
   R^{14′} is hydrogen;
   R¹⁵ is selected from hydrogen; aminoC₁-C₂alkyl; aminocarbonylmethyl; and methyl;
   R^{15′} is hydrogen; and
   R^{15″} is hydrogen; aminocarbonyl; carboxy; or C(O)NHCHR¹⁶R^{16′}; wherein
      R¹⁶ is hydrogen; and
      R^{16′} is hydrogen or ethyl.

Another aspect of the present disclosure provides a pharmaceutical composition comprising a compound of Formula (I), or a pharmaceutically acceptable salt thereof.

An additional aspect of the present disclosure provides a method of enhancing, stimulating, and/or increasing an immune response in a subject in need thereof, wherein the method comprises administering to the subject a therapeutically effective amount of a compound of Formula (I), or a pharmaceutically acceptable salt thereof.

In another aspect, the present disclosure provides a method of inhibiting growth, proliferation, or metastasis of cancer cells in a subject in need thereof, said method comprising administering to the subject a therapeutically effective amount a compound of formula (I), or a pharmaceutically acceptable salt thereof. In some aspects, the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

In another aspect the present disclosure provides a method of treating an infectious disease in a subject in need thereof, the method comprising administering to the subject a therapeutically effective amount of a compound of formula (I), or a pharmaceutically acceptable salt thereof. In some aspects, the infectious disease is caused by a virus. In a second aspect the virus is selected from HIV, Hepatitis A, Hepatitis B, Hepatitis C, herpes viruses, and influenza.

In an another aspect the present disclosure provides a method of treating septic shock in a subject in need thereof, the method comprising administering to the subject a therapeutically effective amount of a compound of formula (I), or a pharmaceutically acceptable salt thereof.

Another aspect of the present disclosure provides a method of blocking the interaction of PD-1 with PD-L1 in a subject, wherein the method comprises administering to the subject a therapeutically effective amount of a compound of Formula (I), or a pharmaceutically acceptable salt thereof.

### DETAILED DESCRIPTION

Unless otherwise indicated, any atom with unsatisfied valences is assumed to have hydrogen atoms sufficient to satisfy the valences.

The singular forms “a,” “an,” and “the” include plural referents unless the context dictates otherwise.

As used herein, the term “or” is a logical disjunction (i.e., and/or) and does not indicate an exclusive disjunction unless expressly indicated such as with the terms “either,” “unless,” “alternatively,” and words of similar effect.

As used herein, the phrase “or a pharmaceutically acceptable salt thereof” refers to at least one compound, or at least one salt of the compound, or a combination thereof. For example, “a compound of Formula (I) or a pharmaceutically acceptable salt thereof” includes, but is not limited to, a compound of Formula (I), two compounds of Formula (I), a pharmaceutically acceptable salt of a compound of Formula (I), a compound of Formula (I) and one or more pharmaceutically acceptable salts of the compound of Formula (I), and two or more pharmaceutically acceptable salts of a compound of Formula (I).

The term “C₁-C₂alkoxy”, as used herein, refers to a C₁-C₂alkyl group attached to the parent molecular moiety through an oxygen atom.

The term “C₁-C₃alkoxy”, as used herein, refers to a C₁-C₃alkyl group attached to the parent molecular moiety through an oxygen atom.

The term “C₁-C₆alkoxy”, as used herein, refers to a C₁-C₆alkyl group attached to the parent molecular moiety through an oxygen atom.

The term “C₁-C₃alkoxyC₁-C₃alkyl”, as used herein, refers to a C₁-C₃alkoxy group attached to the parent molecular moiety through a C₁-C₃alkyl group.

The term “C₁-C₆alkoxyC₁-C₆alkyl”, as used herein, refers to a C₁-C₆alkoxy group attached to the parent molecular moiety through a C₁-C₆alkyl group.

The term “alkyl”, as used herein, refers to a group derived from a straight or branched chain saturated hydrocarbon contain carbon atoms. The term “alkyl” may be proceeded by “C_{#}-C_{#}” wherein the #is an integer and refers to the number of carbon atoms in the alkyl group. For example, C₁-C₂alkyl contains one to two carbon atoms and C₁-C₃alkyl contains one to three carbon atoms.

The term “C₂-C₆alkenyl”, as used herein, refers to a group derived from a straight or branched chain hydrocarbon contain one or more carbon-carbon double bonds containing two to six carbon atoms.

The term “C₂-C₆alkenyl”, as used herein, refers to a group derived from a straight or branched chain hydrocarbon contain one or more carbon-carbon double bonds containing two to six carbon atoms.

The term “C₂-C₆alkenyloxy”, as used herein, refers to a C₂-C₆alkenyl group attached to the parent molecular moiety through an oxygen atom.

The term “C₁-C₃alkylamino,” as used herein, refers to —NHR, wherein R is a C₁-C₃alkyl group.

The term “C₁-C₃alkylaminosulfonyl,” as used herein, refers to a C₁-C₃alkylamino group attached to the parent molecular moiety through an SO₂ group.

The term “C₁-C₃alkylcarbonyl,” as used herein, refers to a C₁-C₃alkyl group attached to the parent molecular moiety through a carbonyl group.

The term “C₁-C₃alkylcarbonylamino,” as used herein, refers to a C₁-C₃alkylcarbonyl group attached to the parent molecular moiety through an amino group.

The term “C₁-C₃alkylcarbonylaminoC₁-C₃alkyl,” as used herein, refers to a C₁-C₃alkylcarbonylamino group attached to the parent molecular moiety through a C₁-C₃alkyl group.

The term “C₁-C₃alkylcarbonylaminoC₁-C₆alkyl,” as used herein, refers to a C₁-C₃alkylcarbonylamino group attached to the parent molecular moiety through a C₁-C₆alkyl group.

The term “C₁-C₃alkylS(O),” as used herein, refers to a C₁-C₃alkyl group attached to the parent molecular moiety through a S(O) group.

The term “C₁-C₃alkylS(O)C₁-C₆alkyl,” as used herein, refers to a C₁-C₃alkylS(O)— group attached to the parent molecular moiety through a C₁-C₆alkyl group.

The term “mono-, di- or tri-C₁-C₆alkylamino group,” as used herein, refers to —NHR, —NR², or —N⁺R³, wherein each R group is independently a C₁-C₆alkyl group.

The term “mono-, di- or tri-C₁-C₆alkylaminoC₁-C₆alkyl,” as used herein, refers to a mono-, di- or tri-C₁-C₆alkylamino group attached to the parent molecular moiety through a C₁-C₆alkyl group.

The term “C₂-C₄alkynyl”, as used herein, refers to a group derived from a straight or branched chain hydrocarbon containing one or more carbon-carbon triple bonds containing two to four carbon atoms.

The term “C₃-C₄alkynyl”, as used herein, refers to a group derived from a straight or branched chain hydrocarbon containing one or more carbon-carbon triple bonds containing three to four carbon atoms.

The term “C₂-C₆alkynyl”, as used herein, refers to a group derived from a straight or branched chain hydrocarbon containing one or more carbon-carbon triple bonds containing two to six carbon atoms.

The term “C₂-C₆alkynoxy”, as used herein, refers to a C₂-C₆alkynyl group attached to the parent molecular moiety through an oxygen atom.

The term “C₁-C₆alkylamino,” as used herein, refers to —NHR^{a}, wherein R^{a} is a C₁-C₆alkyl group.

The term “C₁-C₆alkylaminoC₁-C₆alkyl”, as used herein, refers to a C₁-C₆alkylamino group attached to the parent molecular moiety through a C₁-C₆alkyl group.

The term “C₁-C₄alkylcarbonylamino,” as used herein, refers to —NHC(O)R^{a}, wherein R^{a} is a C₁-C₄alkyl group.

The term “C₁-C₆alkylcarbonylamino,” as used herein, refers to —NHC(O)R^{a}, wherein R^{a} is a C₁-C₆alkyl group.

The term “C₃-C₆cycloalkylcarbonylamino,” as used herein, refers to —NHC(O)R^{a}, wherein R^{a} is a C₃-C₆cycloalkyl group.

The term “C₁-C₄alkylcarbonylaminoethyl”, as used herein, refers to a C₁-C₄alkylcarbonylamino group attached to the parent molecular moiety through an ethylene group.

The term “C₁-C₆alkylcarbonylaminoC₁-C₆alkyl”, as used herein, refers to a C₁-C₆alkylcarbonylamino group attached to the parent molecular moiety through a C₁-C₆alkyl group.

The term “C₃-C₆cycloalkylcarbonylaminoC₁-C₆alkyl”, as used herein, refers to a C₃-C₆cycloalkylcarbonylamino attached to the parent molecular moiety through a C₁-C₆alkyl group.

The term “C₁-C₆alkylcarbonyloxy,” as used herein, refers to a —OC(O)R^{a}, wherein R^{a} is C₁-C₆alkylcarbonyl group.

The term “C₁-C₆alkylcarbonyloxyC₁-C₆alkyl,” as used herein, refers to a C₁-C₆alkylcarbonyloxy group attached to the parent molecular moiety through a C₁-C₆alkyl group.

The term “C₁-C₆alkylcarbonyloxyC₁-C₆alkoxy”, as used herein, refers to a C₁-C₆alkylcarbonyloxyC₁-C₆alkyl group attached to the parent molecular moiety through an oxygen atom.

The term “C₁-C₆alkylcarbonylaminoC₁-C₆alkylthio,” as used herein, refers to a C₁-C₆alkylcarbonylaminoC₁-C₆alkyl group attached to the parent molecular moiety through a sulfur atom.

The term “C₁-C₆alkylcarbonylaminoC₁-C₆alkylthioC₁-C₆alkyl”, as used herein, refers to a C₁-C₆alkylcarbonylaminoC₁-C₆alkylthio group attached to the parent molecular moiety through a C₁-C₆alkyl group.

The term “C₃-C₈cycloalkyl”, as used herein, refers to a C₃-C₈cyclo group attached to the parent molecular moiety through an alkyl group.

The term “(C₃-C₈cycloalkyl)carbonyl,” as used herein, refers to a C₃-C₈cycloalkyl group attached to the parent molecular moiety through a carbonyl group.

The term “(C₃-C₈cycloalkyl)carbonylamino,” as used herein, refers to a (C₃-C₈cycloalkyl)carbonyl group attached to the parent molecular moiety through an amino group.

The term “(C₃-C₈cycloalkyl)carbonylaminoC₁-C₃alkyl,” as used herein, refers to a (C₃-C₈cycloalkyl)carbonylamino group attached to the parent molecular moiety through a C₁-C₃alkyl group.

The term “(C₃-C₈cycloalkyl)carbonylaminoC₁-C₃alkylaryl,” as used herein, refers to a (C₃-C₈cycloalkyl)carbonylaminoC₁-C₃alkyl group attached to the parent molecular moiety through an aryl group.

The term “(C₃-C₈cycloalkyl)carbonylaminoC₁-C₃alkylarylC₁-C₃alkyl,” as used herein, refers to a (C₃-C₈cycloalkyl)carbonylaminoC₁-C₃alkylaryl group attached to the parent molecular moiety through a C₁-C₃alkyl group.

The term “(C₃-C₈cycloalkyl)oxy,” as used herein, refers to a C₃-C₈cycloalkyl group attached to the parent molecular moiety through an oxygen atom.

The term “C₃-C₆cycloalkylmethyl”, as used herein, refers to a C₃-C₆cycloalkyl group attached to the parent molecular moiety through a methylene group.

The term “C₃-C₆cycloalkylC₁-C₃alkyl,” as used herein, refers to a C₃-C₆cycloalkyl group attached to the parent molecular moiety through a C₁-C₃alkyl group.

The term “C₃-C₈cycloalkylC₁-C₆alkyl,” as used herein, refers to a C₃-C₈cycloalkyl group attached to the parent molecular moiety through a C₁-C₆alkyl group.

The term “C₃-C₈cycloalkylC₁-C₆alkylfluoroC₁-C₆alkyl”, as used herein, refers to a C₃-C₈cycloalkylC₁-C₆alkyl group attached to the parent molecular moiety through a fluoroC₁-C₆alkyl group.

The term “C₃-C₆cycloalkylcarbonyl,” as used herein, refers to a C₃-C₆cycloalkyl group attached to the parent molecular moiety through a carbonyl group.

The term “C₃-C₆cycloalkylcarbonylamino,” as used herein, refers to a C₃-C₆cycloalkylcarbonyl group attached to the parent molecular moiety through an amino group.

The term “C₃-C₆cycloalkylcarbonylaminoC₁-C₆alkyl,” as used herein, refers to a C₃-C₆cycloalkylcarbonylamino group attached to the parent molecular moiety through a C₁-C₆alkyl group.

The term “fluoroC₁-C₆alkyl,” as used herein, refers to a C₁-C₆alkyl group substituted with one, two, or three fluoro groups.

The term “fluoroC₁-C₆alkylheterocyclylsulfonyl,” as used herein, refers to a heterocyclylsulfonyl group substituted with a fluoroC₁-C₆alkyl group.

The term “C₁-C₃alkylsulfonyl,” as used herein, refers to a C₁-C₃alkyl group attached to the parent molecular moiety through an SO₂ group.

The term “C₁-C₃alkylsulfonylamino,” as used herein, refers to a C₁-C₃alkylsulfonyl group attached to the parent molecular moiety through an amino group.

The term “C₁-C₃alkylsulfonylaminocarbonyl,” as used herein, refers to a C₁-C₃alkylsulfonylamino group attached to the parent molecular moiety through a carbonyl group.

The term “C₁-C₃alkylsulfonylaminocarbonylC₁-C₃alkyl,” as used herein, refers to a C₁-C₃alkylsulfonylaminocarbonyl group attached to the parent molecular moiety through a C₁-C₃alkyl group.

The term C₁-C₆alkylthio,” as used herein, refers to a C₁-C₆alkyl group attached to the parent molecular moiety through a sulfur atom.

The term “C₁-C₆alkylthioC₁-C₆alkyl”, as used herein, refers to a C₁-C₆alkylthio group attached to the parent molecular moiety through a C₁-C₆alkyl group.

The term “C₁-C₆alkylNHC₁-C₆alkyl”, as used herein, refers to a C₁-C₆alkylNH group attached to the parent molecular moiety through a C₁-C₆alkyl group.

The term “amino”, as used herein, refers to —NH₂. The term “aminomethyl”, as used herein, refers to an amino group attached to the parent molecular moiety through a methyl group.

The term “aminoethyl”, as used herein, refers to an amino group attached to the parent molecular moiety through a ethyl group.

The term “aminoethoxy”, as used herein refers to an amino group attached to the parent molecular moiety through a ethoxy group.

The term “aminocarbonyl”, as used herein, refers to an amino group attached to the parent molecular moiety through a carbonyl group. The term “aminopentanyl”, as used herein refers to an amino group attached to the parent molecular moiety through a pentanyl group.

The term “aminocarbonylC₁-C₂alkyl”, as used herein, refers to (CH₂)ₓC(O)NH₂, wherein x is 1 or 2.

The term “aminocarbonylC₁-C₃alkyl”, as used herein, refers to an aminocarbonyl group attached to the parent molecular moiety through a C₁-C₃alkyl group.

The term “aminocarbonylC₁-C₆alkyl,” as used herein, refers to an aminocarbonyl group attached to the parent molecular moiety through a C₁-C₆alkyl group.

The term “aminocarbonylmethyl”, as used herein, refers to —CH₂C(O)NH₂.

The term “aminocarbonylethyl”, as used herein, refers to (CH₂)₂C(O)NH₂.

The term “aminoC₁-C₂alkyl”, as used herein, refers to an amino group attached to the parent molecular moiety through a C₁-C₂alkyl group.

The term “aminoC₁-C₃alkyl”, as used herein, refers to an amino group attached to the parent molecular moiety through a C₁-C₃alkyl group.

The term “aminoC₁-C₄alkyl”, as used herein, refers to an amino group attached to the parent molecular moiety through a C₁-C₄alkyl group.

The term “aminoC₁-C₅alkyl”, as used herein, refers to an amino group attached to the parent molecular moiety through a C₁-C₅alkyl group.

The term “aminoC₁-C₆alkyl”, as used herein, refers to an amino group attached to the parent molecular moiety through a C₁-C₆alkyl group.

The term “aminoC₃-C₄alkyl”, as used herein, refers to an amino group attached to the parent molecular moiety through a C₃-C₄alkyl group.

The term “aminoC₁-C₆alkoxy”, as used herein, refers to an amino group attached to the parent molecular moiety through a C₁-C₆alkoxy group.

The term “aminoC₂-C₆alkoxy”, as used herein, refers to an amino group attached to the parent molecular moiety through a C₂-C₆alkoxy group.

The term “aminocarbonylC₁-C₆alkyl”, as used herein, refers to an aminocarbonyl group attached to the parent molecular moiety through a C₁-C₆alkyl group.

The term “aminopropyl”, as used herein, refers to a amino group attached to the parent molecular moiety through a propyl group.

The term “aryl,” as used herein, refers to a phenyl group, or a bicyclic fused ring system wherein one or both of the rings is a phenyl group. Bicyclic fused ring systems consist of a phenyl group fused to a four- to six-membered aromatic or non-aromatic carbocyclic ring. The aryl groups of the present disclosure can be attached to the parent molecular moiety through any substitutable carbon atom in the group. Representative examples of aryl groups include, but are not limited to, indanyl, indenyl, naphthyl, phenyl, and tetrahydronaphthyl.

The term “aryloxy”, as used herein, refers to an aryl group attached to the parent molecular moiety though an oxygen atom.

The term “arylmethyl”, as used herein, refers to an aryl group attached to the parent molecular moiety through a methyl group.

The term “arylC₁-C₂alkyl”, as used herein, refers to an aryl group attached to the parent molecular moiety through a C₁-C₂alkyl group.

The term “arylC₁-C₃alkyl,” as used herein, refers to an aryl group attached to the parent molecular moiety through a C₁-C₃alkyl group.

The term “arylC₁-C₃alkylcarbonyl,” as used herein, refers to an arylC₁-C₃alkyl group attached to the parent molecular moiety through a carbonyl group.

The term “arylC₁-C₆alkoxy”, as used herein, refers to an aryl group attached to the parent molecular moiety through a C₁-C₆alkoxy group.

The term “arylC₁-C₆alkyl”, as used herein, refers to an aryl group attached to the parent molecular moiety through a C₁-C₆alkyl group.

The term “aryl-aryl,” as used herein, refers to an aryl group attached to the parent molecular moiety through a second aryl group.

The term “aryl-arylC₁-C₃alkyl,” as used herein, refers to an aryl-aryl group attached to the parent molecular moiety through a C₁-C₃alkyl group. The term “biphenylC₁-C₆alkyl”, as used herein, refers to a biphenyl group attached to the parent molecular moiety through a C₁-C₆alkyl group. The biphenyl group can be attached to the alkyl moiety through any substitutable atom in the group.

The term “aryl-arylmethyl”, as used herein, refers to an aryl-aryl group attached to the parent molecular moiety through a methylene group.

The term “aryl-arylC₁-C₃alkyl”, as used herein, refers to an aryl-aryl group attached to the parent molecular moiety through a C₁-C₃alkyl group.

The term “aryl-heteroarylC₁-C₃alkyl”, as used herein, refers to an aryl-heteroaryl group attached to the parent molecular moiety through a C₁-C₃alkyl group.

The term “arylsulfonyl,” as used herein, refers to an aryl group attached to the parent molecular moiety through an SO₂ group.

The term “arylsulfonylamino,” as used herein, refers to an arylsulfonyl group attached to the parent molecular moiety through an amino group.

The term “arylsulfonylaminocarbonyl,” as used herein, refers to an arylsulfonylamino group attached to the parent molecular moiety through a carbonyl group.

The term “arylsulfonylaminocarbonylC₁-C₃alkyl,” as used herein, refers to an arylsulfonylaminocarbonyl group attached to the parent molecular moiety through a C₁-C₃alkyl group.

The term “azidoC₁-C₂alkyl”, as used herein, refers to an azido group attached to the parent molecular moiety through a C₁-C₂alkyl group.

The term “benzyloxy”, as used herein, refers to a benzyl group attached to the parent molecular moiety through an oxygen atom.

The term “bis(carboxyC₁-C₃alkyl)amino,” as used herein, refers to —NR₂, wherein each R group is a (carboxyC₁-C₃alkyl group.

The term “bis(carboxyC₁-C₃alkyl)aminoC₁-C₃alkyl,” as used herein, refers to a bis(carboxyC₁-C₃alkyl)amino group attached to the parent molecular moiety through a C₁-C₃alkyl group.

The term “bis(carboxyC₁-C₃alkyl)aminoC₁-C₃alkylcarbonyl,” as used herein, refers to a bis(carboxyC₁-C₃alkyl)aminoC₁-C₃alkyl group attached to the parent molecular moiety through a carbonyl group.

The term “bis(carboxyC₁-C₃alkyl)aminoC₁-C₃alkylcarbonylamino,” as used herein, refers to a bis(carboxyC₁-C₃alkyl)aminoC₁-C₃alkylcarbonyl group attached to the parent molecular moiety through an amino group.

The term “bis(carboxyC₁-C₃alkyl)aminoC₁-C₃alkylcarbonylaminoC₁-C₃alkyl,” as used herein, refers to a bis(carboxyC₁-C₃alkyl)aminoC₁-C₃alkylcarbonylamino group attached to the parent molecular moiety through a C₁-C₃alkyl group.

The term “butylcarbonylaminoethyl”, as used herein, refers to a butylcarbonylamino group attached to the parent molecular moiety through an ethylene group.

The term “butylcarbonylamino,” as used herein, refers to —NHC(O)R^{a}, wherein R^{a} is butyl.

The term “butoxycarbonylmethoxy”, as used herein refers to a butoxycarbonylmethyl group attached to the parent molecular moiety through an oxygen atom.

The term “butoxycarbonylmethyl,” as used herein, refers to —(CH₂)CO₂R^{a}, wherein R^{a} is butyl.

The term “carbamidylC₁-C₆alkyl”, as used herein refers to a carbamidyl group attached to the parent molecular moiety through a C₁-C₆alkyl group.

The term “carbamidylC₃-C₄alkyl”, as used herein refers to a carbamidyl group attached to the parent molecular moiety through a C₃-C₄alkyl group.

The term “carbonyl”, as used herein, refers to —C(O)—.

The term “carboxy”, as used herein, refers to —CO₂H.

The term “carboxyC₁-C₂alkyl”, as used herein, refers to a C₁-C₂alkyl group substituted with one or two carboxy groups.

The term “carboxyC₁-C₃alkyl”, as used herein, refers to a —C₁-C₃alkyl group substituted with one or two carboxy groups.

The term “carboxyC₁-C₆alkyl”, as used herein, refers to a C₁-C₆alkyl group substituted with one or two carboxy groups.

The term “carboxyC₁-C₃alkylamino,” as used herein, refers to a carboxyC₁-C₃alkyl attached to the parent molecular moiety through an amino group.

The term “carboxyC₁-C₃alkylaminocarbonyl,” as used herein, refers to a carboxyC₁-C₃alkylamino attached to the parent molecular moiety through a carbonyl group.

The term “carboxyC₁-C₃alkylaminocarbonylC₁-C₃alkyl,” as used herein, refers to a carboxyC₁-C₃alkylaminocarbonyl attached to the parent molecular moiety through a C₁-C₃alkyl group.

The term “carboxyC₁-C₃alkylcarbonyl,” as used herein, refers to a carboxyC₁-C₃alkyl group attached to the parent molecular moiety through a carbonyl group.

The term “carboxyC₁-C₆alkylcarbonyl,” as used herein, refers to a carboxyC₁-C₃alkyl group attached to the parent molecular moiety through a carbonyl group.

The term “carboxyC₁-C₃alkylcarbonylamino,” as used herein, refers to a carboxyC₁-C₃alkylcarbonyl group attached to the parent molecular moiety through an amino group.

The term “carboxyC₁-C₆alkylcarbonylamino,” as used herein, refers to a carboxyC₁-C₃alkylcarbonyl group attached to the parent molecular moiety through an amino group.

The term “carboxyC₁-C₃alkylcarbonylaminoC₁-C₃alkyl,” as used herein, refers to a carboxyC₁-C₃alkylcarbonylamino group attached to the parent molecular moiety through a C₁-C₃alkyl group.

The term “carboxyC₁-C₆alkylcarbonylaminoC₁-C₃alkyl,” as used herein, refers to a carboxyC₁-C₆alkylcarbonylamino group attached to the parent molecular moiety through a C₁-C₃alkyl group.

The term “carboxyC₁-C₆alkoxy”, as used herein, refers to a carboxy group attached to the parent molecular moiety through a C₁-C₆alkoxy group.

The term “carboxyaryl”, as used herein refers to a carboxy group attached to the parent molecular moiety through an aryl group.

The term “carboxymethyl”, as used herein, refers to refers to a carboxy group attached to the parent molecular moiety through a methyl group.

The term “carboxyethyl”, as used herein, refers to a carboxy group attached to the parent molecular moiety through a ethyl group.

The term “carboxymethoxy”, as used herein, refers to a carboxy group attached to the parent molecular moiety through a methoxy group.

The term “carboxymethoxyC₁-C₂alkyl”, as used herein, refers to (CH₂)₂₀CH₂CO₂H.

The term “carboxyC₁-C₆alkoxyC₁-C₆alkyl”, as used herein refers to a carboxyC₁-C₆alkoxy group attached to the parent molecular moiety through a C₁-C₆alkyl group.

The term “carboxypropyl”, as used herein, refers to a carboxy group attached to the parent molecular moiety through a propyl group.

The term “cyano”, as used herein, refers to —CN.

The term “cyanomethyl”, as used herein, refers to a cyano group attached to the parent molecular moiety through a methyl group.

The term “cyanoethyl”, as used herein, refers to a cyano group attached to the parent molecular moiety through an ethyl group.

The term “cyanoC₁-C₆alkyl”, as used herein, refers to a cyano group attached to the parent molecular moiety through a C₁-C₆alkyl.

The term “cyclohexylmethyl”, as used herein, refers to a cyclohexyl group attached to the parent molecular moiety through a methyl group.

The term “cyclopropylmethyl”, as used herein refers to a cyclopropyl group attached to the parent molecular moiety though a methyl group.

The term “cyclopropylcarbonylaminopropyl”, as used herein refers to a cyclopropylcarbonylamino group attached to the parent molecular moiety through a propylene group.

The term “cyclopropylcarbonylamino,” as used herein, refers to —NHC(O)R^{a}, wherein R^{a} is a cyclopropyl group.

The term “cyclohexyl”, as used herein, refers to a group derived from a monocyclic or bicyclic hydrocarbon containing six carbon atoms that is completely saturated and has a single point of attachment to the parent molecular moiety.

The term “biphenylmethyl”, as used herein refers to a biphenyl group attached to the parent molecular moiety through a methylene group.

The term “dimethylaminosulfonyl,” as used herein, refers to a dimethylamino group attached to the parent molecular moiety through a sulfonyl group.

The term “dimethylaminosulfonylamino,” as used herein, refers to a dimethylaminosulfonyl group attached to the parent molecular moiety through an amino group.

The term “dimethylaminosulfonylaminocarbonyl,” as used herein, refers to a dimethylaminosulfonylamino group attached to the parent molecular moiety through a carbonyl group.

The term “dimethylaminosulfonylaminocarbonylC₁-C₃alkyl,” as used herein, refers to a dimethylaminosulfonylaminocarbonyl group attached to the parent molecular moiety through a C₁-C₃alkyl group.

The term “guanidinylC₁-C₆alkyl”, as used herein refers to a guandinyl group attached to the parent molecular moiety through a C₁-C₆alkyl group.

The term “guanidinylC₃-C₄alkyl”, as used herein refers to a guandinyl group attached to the parent molecular moiety through a C₃-C₄alkyl group.

The terms “halo” and “halogen”, as used herein, refer to F, Cl, Br, or I.

The term “haloC₁-C₃alkyl,” as used herein, refers to a C₁-C₃alkyl group substituted with one, two, or three halogen atoms.

The term “haloC₁-C₆alkylcarbonyl,” as used herein, refers to a haloC₁-C₆alkyl group attached to the parent molecular moiety through a carbonyl group.

The term “haloC₁-C₆alkylcarbonylamino,” as used herein, refers to a haloC₁-C₆alkylcarbonyl group attached to the parent molecular moiety through an amino group.

The term “haloC₁-C₆alkylcarbonylaminoC₁-C₃alkyl,” as used herein, refers to a haloC₁-C₆alkylcarbonylamino group attached to the parent molecular moiety through a C₁-C₃alkyl group.

The term “heteroaryl”, as used herein, refers to a monocyclic, bicyclic, and tricyclic ring system having a total of five to fourteen ring members, wherein said ring system has a single point of attachment to the rest of the molecule, at least one ring in the system is aromatic, at least one ring in the system contains one or more heteroatoms independently selected from nitrogen, oxygen, sulfur or phosphorus, and wherein each ring in the system contains 4 to 7 ring members. The term “heteroaryl” may be used interchangeably with the term “heteroaryl ring” or the term “heteroaromatic.”

The term “heteroarylamino,” as used herein, refers to a heteroaryl group attached to the parent molecular moiety through an amino group.

The term “heteroarylaminocarbonyl,” as used herein, refers to a heteroarylamino group attached to the parent molecular moiety through a carbonyl group.

The term “heteroarylaminocarbonylC₁-C₃alkyl,” as used herein, refers to a heteroarylaminocarbonyl group attached to the parent molecular moiety through a C₁-C₃alkyl group.

The term “aryl-heteroaryl,” as used herein, refers to an aryl group attached to the parent molecular moiety through a heteroaryl group.

The term “heteroaryl-aryl,” as used herein, refers to a heteroaryl group attached to the parent molecular moiety through an aryl group.

The term “heteroaryl-heteroaryl,” as used herein, refers to a heteroaryl group attached to the parent molecular moiety through a heteroaryl group.

The term “heteroaryl-arylC₁-C₃alkyl,” as used herein, refers to a heteroaryl-aryl group attached to the parent molecular moiety through a C₁-C₃alkyl group.

The term “aryl-heteroarylC₁-C₃alkyl,” as used herein, refers to a aryl-heteroaryl group attached to the parent molecular moiety through a C₁-C₃alkyl group.

The term “heteroaryl-heteroarylC₁-C₃alkyl,” as used herein, refers to a heteroaryl-heteroaryl group attached to the parent molecular moiety through a C₁-C₃alkyl group.

The term “heteroarylmethyl”, as used herein, refers to a heteroaryl group attached to the parent molecular moiety through a methyl group.

The term “heteroarylC₁-C₂alkyl”, as used herein, refers to a heteroaryl group attached to the parent molecular moiety through a C₁-C₂alkyl group.

The term “heteroarylC₁-C₃alkyl”, as used herein, refers to a heteroaryl group attached to the parent molecular moiety through a C₁-C₃alkyl group.

The term “heteroarylC₁-C₆alkyl”, as used herein, refers to a heteroaryl group attached to the parent molecular moiety through a C₁-C₆alkyl group.

The term “heteroarylC₁-C₆alkyl”, as used herein, refers to a heteroaryl group attached to the parent molecular moiety through a C₁-C₆alkyl group.

The term “heteroarylC₁-C₃alkylcarbonyl,” as used herein, refers to a heteroarylC₁-C₃alkyl group attached to the parent molecular moiety through a carbonyl group.

The term “heteroaryl-heteroarylC₁-C₃alkyl”, as used herein, refers heteroaryl-heteroaryl group attached to the parent molecular moiety through a C₁-C₃alkyl group.

The term “heteroaryl-arylC₁-C₃alkyl”, as used herein, refers to a heteroaryl-aryl group attached to the parent molecular moiety through a C₁-C₃alkyl group.

The term “heterocyclyl”, as used herein, refers to a five-, six-, or seven-membered ring containing one, two, or three heteroatoms independently selected from nitrogen, oxygen, and sulfur. The five-membered ring has zero to two double bonds and the six- and seven-membered rings have zero to three double bonds. The term “heterocyclyl” also includes bicyclic groups in which the heterocyclyl ring is fused to a four- to six-membered aromatic or non-aromatic carbocyclic ring or another monocyclic heterocyclyl group. The heterocyclyl groups of the present disclosure are attached to the parent molecular moiety through a carbon atom in the group. Examples of heterocyclyl groups include, but are not limited to, benzothienyl, furyl, imidazolyl, indolinyl, indolyl, isothiazolyl, isoxazolyl, morpholinyl, oxazolyl, piperazinyl, piperidinyl, pyrazolyl, pyridinyl, pyrrolidinyl, pyrrolopyridinyl, pyrrolyl, thiazolyl, thienyl, and thiomorpholinyl.

The term “heterocyclylmethyl”, as used herein, refers to a heterocyclyl group attached to the parent molecular moiety through a methyl group.

The term “heterocyclylC₁-C₆alkyl”, as used herein, refers to a heterocyclyl group attached to the parent molecular moiety through a C₁-C₆alkyl group.

The term “heterocyclylsulfonyl,” as used herein, refers to a heterocyclyl group attached to the parent molecular moiety through an SO₂ group.

The term “hydroxy”, as used herein, refers to —OH.

The term “hydroxymethyl”, as used herein, refers to a hydroxy group attached to the parent molecular moiety through a methyl group.

The term “hydroxyethyl”, as used herein, refers to a hydroxy group attached to the parent molecular moiety through an ethyl group.

The term “hydroxypropyl”, as used herein, refers to a hydroxy group attached to the parent molecular moiety through a propyl group.

The term “hydroxyC₂-C₆alkenyl,” as used herein, refers to a hydroxy group attached to the parent molecular moiety through a C₂-C₆alkenyl group.

The term “hydroxyC₁-C₂alkyl”, as used herein, refers to a hydroxy group attached to the parent molecular moiety through a C₁-C₂alkyl group.

The term “hydroxyC₁-C₃alkyl”, as used herein, refers to a hydroxy group attached to the parent molecular moiety through a C₁-C₃alkyl group.

The term “hydroxyC₁-C₄alkyl”, as used herein, refers to a hydroxy group attached to the parent molecular moiety through a C₁-C₄alkyl group.

The term “hydroxyC₁-C₆alkyl”, as used herein, refers to a hydroxy group attached to the parent molecular moiety through a C₁-C₆alkyl group.

The term “hydroxyC₂-C₃alkyl”, as used herein, refers to a hydroxy group attached to the parent molecular moiety through a C₂-C₃alkyl group. The term “hydroxyC₁-C₆alkylcarbonyl,” as used herein, refers to a hydroxyC₁-C₆alkyl group attached to the parent molecular moiety through a carbonyl group.

The term “hydroxyC₁-C₆alkylcarbonylamino,” as used herein, refers to a hydroxyC₁-C₆alkylcarbonyl group attached to the parent molecular moiety through an amino group.

The term “hydroxyC₁-C₆alkylcarbonylaminoC₁-C₃alkyl,” as used herein, refers to a hydroxyC₁-C₆alkylcarbonylamino group attached to the parent molecular moiety through a C₁-C₃alkyl group.

The term “methoxy”, as used herein, refers to —OCH₃

The term “methoxymethyl”, as used herein, refers to a methoxy group attached to the parent molecular moiety through a methyl group.

The term “methylamino,” as used herein, refers to —NHCH₃.

The term “methylcarbonylamino”, as used herein, refers to —NHC(O)CH₃

The term “methoxyC₁-C₂alkyl”, as used herein refers to a methoxy group attached to the parent molecular moiety through a C₁-C₂alkyl group.

The term “methylaminoC₁-C₆alkyl”, as used herein refers to a methylamino group attached to the parent molecular moiety through a C₁-C₆alkyl group.

The term “methylthioethyl”, as used herein refers to a methylthio group attached to the parent molecular moiety through an ethylene group.

The term “methylcarbonylaminobutyl”, as used herein, refers to —(CH₂)₄NHC(O)CH₃.

The term “methylcarbonylaminoC₃-C₄alkyl”, as used herein, refers to a methylcarbonylamino group attached to the parent molecular moiety through a C₃-C₄alkyl group.

The term “methylaminoethyl”, as used herein refers to —(CH₂)₂NHCH₃.

The term “methylcarbonylaminoethyl”, as used herein refers to a methylcarbonylamino group attached to the parent molecular moiety through an ethylene group.

The term “methylcarbonylaminomethylthiomethyl”, as used herein refers to a methylcarbonylaminomethylthio group attached to the parent molecular moiety through a methylene group.

The term “methylcarbonylaminomethylthio”, as used herein refers to a methylcarbonylaminomethyl group attached to the parent molecular moiety through a sulfur atom.

The term “methylcarbonylaminomethyl,” as used herein, refers to a methylcarbonyl amino group attached to the parent molecular moiety through a methylene group.

The term “nitro”, as used herein, refers to —NO₂.

The term “phenylcarbonyl,” as used herein, refers to a phenyl group attached to the parent molecular moiety through a carbonyl group.

The term “phenylmethyl”, as used herein, refers to a phenyl group attached to the parent molecular moiety through a methyl group.

The term “propynoxyl”, as used herein, refers to a three-membered carbon chain containing a carbon-carbon double bond attached to the parent molecular moiety through an oxygen atom.

The term “propenoxyl”, as used herein, refers to a three-membered carbon chain containing a carbon-carbon triple bond attached to the parent molecular moiety through an oxygen atom.

The term “pyridylmethyl”, as used herein refers to a pyridyl group attached to the parent molecular moiety through a methyl group.

The term “imidazolylmethyl”, as used herein, refers to an imidzolyl group attached to the parent molecular moiety through a methyl group.

The term “indolylmethyl”, as used herein refers to an indolyl group attached to the parent molecular moiety through a methyl group.

The term “R⁷NHC₁-C₆alkyl,” as used herein, refers to a R⁷⁰NH group attached to the parent molecular moiety through a C₁-C₆alkyl group.

The term “tetrazolylC₁-C₃alkyl,” as used herein, refers to a tetrazolyl group attached to the parent molecular moiety through a C₁-C₃alkyl group.

As used herein, “hyperproliferative disease” refers to conditions wherein cell growth is increased over normal levels. For example, hyperproliferative diseases or disorders include malignant diseases (e.g., esophageal cancer, colon cancer, biliary cancer) and non-malignant diseases (e.g., atherosclerosis, benign hyperplasia, and benign prostatic hypertrophy).

The term “immune response” refers to the action of, for example, lymphocytes, antigen presenting cells, phagocytic cells, granulocytes, and soluble macromolecules that results in selective damage to, destruction of, or elimination from the human body of invading pathogens, cells or tissues infected with pathogens, cancerous cells, or, in cases of autoimmunity or pathological inflammation, normal human cells or tissues.

The terms “Programmed Death Ligand 1”, “Programmed Cell Death Ligand 1”, “PD-L1”, “PDL1”, “hPD-L1”, “hPD-L1”, and “B7-H1” are used interchangeably, and include variants, isoforms, species homologs of human PD-L1, and analogs having at least one common epitope with PD-L1. The complete PD-L1 sequence can be found under GENBANK® Accession No. NP_054862.

The terms “Programmed Death 1”, “Programmed Cell Death 1”, “Protein PD-1”, “PD-1”, “PD1”, “hPD-1” and “hPD-1” are used interchangeably, and include variants, isoforms, species homologs of human PD-1, and analogs having at least one common epitope with PD-1. The complete PD-1 sequence can be found under GENBANK® Accession No. U64863.

The term “treating” refers to inhibiting the disease, disorder, or condition, i.e., arresting its development; and (iii) relieving the disease, disorder, or condition, i.e., causing regression of the disease, disorder, and/or condition and/or symptoms associated with the disease, disorder, and/or condition.

The present disclosure is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the disclosure can generally be prepared by conventional techniques known to those skilled in the art or by processes analogous to those described herein, using an appropriate isotopically-labeled reagent in place of the non-labeled reagent otherwise employed. Such compounds can have a variety of potential uses, for example as standards and reagents in determining biological activity. In the case of stable isotopes, such compounds can have the potential to favorably modify biological, pharmacological, or pharmacokinetic properties.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

Various aspect of the disclosure are described in greater detail below.

### Compounds of Formula (I)

In a first aspect, the present disclosure provides a compound of Formula (I):

or a pharmaceutically acceptable salt thereof, wherein:
R¹ is selected from C₁-C₆alkyl; mono-, di- or tri-C₁-C₆alkylaminoC₁-C₆alkyl; aminoC₁-C₆alkyl; aminocarbonylC₁-C₆alkyl; arylC₁-C₆alkyl; carbamidylC₁-C₆alkyl; cyanoC₁-C₆alkyl; C₃-C₆cycloalkylcarbonylaminoC₁-C₆alkyl; guanidinylC₁-C₆alkyl; heteroarylC₁-C₆alkyl; heterocyclylC₁-C₆alkyl; hydroxyC₁-C₆alkyl; and H₂NC(X) , where X is O or NH, and represents an azetidine, pyrrolidine, or piperidine ring; and wherein the aryl part of the arylC₁-C₆alkyl and the heteroaryl part of the heteroarylC₁-C₆alkyl a optionally substituted with one, two, three, four, or five groups independently selected from aminoC₂-C₆alkoxy, aminoC₁-C₆alkyl, aminocarbonyl, carboxy, carboxyC₁-C₆alkoxy halo, hydroxy, and nitro;
R² is selected from C₂-C₆alkenyl; C₁-C₆alkylcarbonylaminoC₁-C₆alkylthioC₁-C₆alkyl; aminocarbonyl C₁-C₆alkyl; arylC₁-C₆alkyl; heteroarylC₁-C₆alkyl; and hydroxyC₁-C₆alkyl; wherein the aryl part of the arylC₁-C₆alkyl is optionally substituted with one, two, three, four, or five groups independently selected from C₂-C₆alkenyl, C₂-C₆alkenyloxy, C₁-C₆alkoxy, C₁-C₆alkyl, C₁-C₆alkylcarbonyloxyC₁-C₆alkoxy, C₂-C₆alkynyloxy, amino, aminoC₁-C₆alkoxy, aminoC₁-C₆alkyl, aminocarbonyl, aryloxy, carboxy, carboxyC₁-C₆alkoxy, cyano, halo, hydroxy, carboxyaryl, nitro, trifluoromethyl, and —OP(O)X¹X², wherein each of X¹ and X² is —OH, —NH₂, or —N(C₁-C₆alkyl)₂;
R³ is selected from aminocarbonylC₁-C₃alkyl; carboxyC₁-C₃alkyl; (OH)₂P(O)OC₁-C₃alkyl; and tetrazolylC₁-C₃alkyl;
R⁴ is selected from arylC₁-C₆alkyl and heteroarylC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, cyano, fluoroC₁-C₆alkyl, and halo;
R⁵ is selected from C₂-C₆alkenyl; C₁-C₆alkyl; C₁-C₆alkylcarbonylaminoC₁-C₆alkylthioC₁-C₆alkyl; aminoC₁-C₆alkyl; aminocarbonylC₁-C₆alkyl; arylC₁-C₆alkyl; carboxyC₁-C₆alkyl; cyanoC₁-C₆alkyl; C₃-C₈cycloalkyl; (C₃-C₈cycloalkyl)C₁-C₆alkyl; and heteroarylC₁-C₆alkyl; and hydroxyC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkyl, fluoroC₁-C₆alkyl, carboxy, aminoC₁-C₆alkyl, aminocarbonyl, and carboxyC₁-C₆alkoxy halo, and hydroxy;
R⁶ is aryl-arylC₁-C₃alkyl, heteroaryl-arylC₁-C₃alkyl, aryl-heteroarylC₁-C₃alkyl, heteroaryl-heteroarylC₁-C₃alkyl, wherein the aryl or the heteroaryl part is optionally substituted with one, two, three, four, or five groups independently selected from C₁-C₆alkylcarbonylamino, aminocarbonyl, fluoroC₁-C₆alkyl, halo, hydroxy, trifluoromethoxy, C₁-C₆alkoxy, C₁-C₆alkoxyC₁-C₆alkyl, carboxyC₁-C₆alkoxyC₁-C₆alkyl, cyanoC₁-C₆alkyl, and arylC₁-C₆alkoxy;
R⁷ is selected from hydrogen; C₂-C₆alkenyl; C₁-C₆alkyl; aminoC₁-C₆alkyl; aminocarbonylC₁-C₆alkyl; carboxyC₁-C₆alkyl; arylC₁-C₆alkyl; C₁-C₆alkylcarbonylaminoC₁-C₆alkylthioC₁-C₆alkyl; C₁-C₆alkylcarbonylaminoC₁-C₆alkyl; heteroarylC₁-C₆alkyl; hydroxyC₁-C₆alkyl; and NH₂C(X)NHC₁-C₆alkyl, where X is O or NH; and wherein the aryl part of the arylC₁-C₆alkyl and the heteroaryl part of the heteroarylC₁-C₆alkyl are optionally substituted with one, two, three, four, or five groups independently selected from aminoC₁-C₆alkyl, aminocarbonyl, carboxy, carboxyC₁-C₆alkoxy, and hydroxy;
R⁸ is selected from C₁-C₆alkyl; aminoC₁-C₆alkyl; carboxyC₁-C₆alkyl; aryl; arylC₁-C₆alkyl; heteroarylC₁-C₆alkyl; and hydroxyC₁-C₆alkyl; wherein the aryl part of the arylC₁-C₆alkyl is optionally substituted with one, two, three, four, or five groups independently selected from halo and hydroxy;
R⁹ is selected from hydrogen; C₁-C₆alkyl; aminoC₁-C₆alkyl; aminocarbonylC₁-C₆alkyl; aryl; arylC₁-C₆alkyl; carboxyC₁-C₆alkyl; C₃-C₈cycloalkyl; C₃-C₈cycloalkylC₁-C₆alkyl; heteroarylC₁-C₆alkyl; hydroxyC₁-C₆alkyl; C₁-C₆alkylthioC₁-C₆alkyl; and NH₂C(X)NHC₁-C₆alkyl, where X is O or NH; and wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amino, carboxyC₁-C₆alkyl, cyano, halo, hydroxy, nitro, and trifluoromethyl;
R¹⁰ is selected from C₁-C₆alkyl; C₁-C₆alkylcarbonylaminoC₁-C₆alkyl; C₁-C₆alkylNHC₁-C₆alkyl; aminoC₁-C₆alkyl; aminocarbonylC₁-C₆alkyl; carboxyC₁-C₆alkyl; hydroxyC₁-C₆alkyl; NH₂C(X)NHC₁-C₆alkyl, where X is O or NH; heteroarylC₁-C₆alkyl; and arylC₁-C₆alkyl; and wherein the aryl part of the arylC₁-C₆alkyl is optionally substituted with one, two, three, four, or five aminoC₁-C₆alkyl groups;
R¹¹ is selected from C₁-C₆alkyl, arylC₁-C₆alkyl, and C₃-C₈cycloalkylC₁-C₆alkyl; wherein the aryl part of the arylC₁-C₆alkyl is optionally substituted with one, two, three, four, or five groups independently selected from C₁-C₆alkyl, halo, and hydroxy;
R¹² is selected from C₂-C₆alkenyl; C₁-C₆alkyl; C₁-C₆alkylcarbonylaminoC₁-C₆alkylthioC₁-C₆alkyl; aminoC₁-C₆alkyl; arylC₁-C₆alkyl; carboxyC₁-C₆alkyl; hydroxyC₁-C₆alkyl; and NH₂C(X)NHC₁-C₆alkyl, where X is O or NH;
R¹³ is selected from C₂-C₆alkenyl; C₁-C₆alkyl; C₁-C₆alkylcarbonylaminoC₁-C₆alkyl; C₁-C₆alkylcarbonylaminoC₁-C₆alkylthioC₁-C₆alkyl; aminoC₁-C₆alkyl; aminocarbonylC₁-C₆alkyl; carboxyC₁-C₆alkyl; cyanoC₁-C₆alkyl; C₃-C₈cycloalkyl; heteroarylC₁-C₆alkyl; hydroxyC₁-C₆alkyl; and NH₂C(X)NHC₁-C₆alkyl, where X is O or NH;
R¹⁴ is aminocarbonyl or —C(O)NR^{14′}CR¹⁵R^{15′}R^{15″}, wherein
R^{14′} is hydrogen, or R¹⁵ and R^{14′}, together with the atoms to which they are attached, form an azetidine, morpholine, piperidine, piperazine, or pyrrolidine ring, wherein each ring is optionally substituted with an amino or a hydroxy group;
R¹⁵ is selected from hydrogen; C₂-C₆alkenyl; C₁-C₆alkyl; C₁-C₆alkylcarbonylaminoC₁-C₆alkyl; C₁-C₆alkylcarbonylaminoC₁-C₆alkylthioC₁-C₆alkyl; aminoC₁-C₆alkyl; aminocarbonylC₁-C₆alkyl; carboxy; carboxyC₁-C₆alkyl; heterocyclyl; hydroxyC₁-C₆alkyl; and NH₂C(X)NHC₁-C₆alkyl, where X is O or NH;
R^{15′} is hydrogen, or R¹⁵ and R^{15′}, together with the atoms to which they are attached, form a C₃-C₈cycloalkyl ring; and
R^{15″} is hydrogen; —C(O)NH₂, or —(CH₂)ₙC(O)NHCHR¹⁶R^{16′}; wherein
n is 0, 1, or 2;
R¹⁶ is selected from hydrogen, C₂-C₆alkynyl, aminoC₁-C₆alkyl, and carboxyC₁-C₆alkyl;
R^{16′} is hydrogen; C₁-C₆alkyl; aminocarbonyl; carboxy; or —C(O)NHCHR¹⁷R^{17′}; wherein
R¹⁷ is hydrogen; and
R¹⁷ is —C(O)NHCHR¹⁸R^{18′}; wherein
R¹⁸ is aminoC₁-C₆alkyl; and
R^{18′} is carboxy.

Those of ordinary skill in the art are aware that an amino acid includes a compound represented by the general structure:

where R and R′ are as discussed herein. Unless otherwise indicated, the term “amino acid” as employed herein, alone or as part of another group, includes, without limitation, an amino group and a carboxyl group linked to the same carbon, referred to as “a” carbon, where R and/or R′ can be a natural or an un-natural side chain, including hydrogen. The absolute “S” configuration at the “a” carbon is commonly referred to as the “L” or “natural” configuration. In the case where both the “R” and the “R′” (prime) substituents equal hydrogen, the amino acid is glycine and is not chiral.

Where not specifically designated, the amino acids described herein can be D- or L-stereochemistry and can be substituted as described elsewhere in the disclosure. It should be understood that when stereochemistry is not specified, the present disclosure encompasses all stereochemical isomeric forms, or mixtures thereof, which possess the ability to inhibit the interaction between PD-1 and PD-L1. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, or direct separation of enantiomers on chiral chromatographic columns. Starting compounds of particular stereochemistry are either commercially available or can be made and resolved by techniques known in the art.

Certain compounds of the present disclosure can exist in different stable conformational forms which may be separable. Torsional asymmetry due to restricted rotation about an asymmetric single bond, for example because of steric hindrance or ring strain, may permit separation of different conformers. The present disclosure includes each conformational isomer of these compounds and mixtures thereof.

The pharmaceutical compounds of the disclosure can include one or more pharmaceutically acceptable salts. A “pharmaceutically acceptable salt” refers to a salt that retains the desired biological activity of the parent compound and does not impart any undesired toxicological effects (see e.g., Berge, S. M. et al., J. Pharm. Sci., 66:1-19 (1977)). The salts can be obtained during the final isolation and purification of the compounds described herein, or separately be reacting a free base function of the compound with a suitable acid or by reacting an acidic group of the compound with a suitable base. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Methods

As demonstrated herein, the compounds of the present disclosure are capable of binding to PD-1, disrupting the interaction between PD-1 and PD-L1, competing with the binding of PD-1 with anti-PD-1 monoclonal antibodies that are known to block the interaction with PD-L1, and enhancing CMV-specific T cell IFNγ secretion. As a result, the compounds of the present disclosure are useful for modifying an immune response, treating diseases such as cancer, stimulating a protective autoimmune response, or to stimulate antigen-specific immune responses (e.g., by co-administration of PD-L1 blocking compounds with an antigen of interest).

Another aspect of the present disclosure is directed to a method of enhancing, stimulating, and/or increasing an immune response in a subject in need thereof, wherein the method comprises administering to the subject a therapeutically effective amount of a compound of Formula (I), or a pharmaceutically acceptable salt thereof. In a first aspect this method further comprises administering an additional agent prior to, after, or simultaneously with the compound of formula (I), compound of formula (I)), or a pharmaceutically acceptable salt thereof. In a second aspect the additional agent is selected from an antimicrobial agent, an antiviral agent, a cytotoxic agent, a TLR7 agonist, a TLR8 agonist, an HDAC inhibitor, and an immune response modifier.

The present disclosure also provides a method of inhibiting growth, proliferation, or metastasis of cancer cells in a subject in need thereof, said method comprising administering to the subject a therapeutically effective amount a compound of formula (I), or a pharmaceutically acceptable salt thereof. In a first aspect of this aspect the cancer is selected from melanoma, renal cell carcinoma, squamous non-small cell lung cancer (NSCLC), non-squamous NSCLC, colorectal cancer, castration-resistant prostate cancer, ovarian cancer, gastric cancer, hepatocellular carcinoma, pancreatic carcinoma, squamous cell carcinoma of the head and neck, carcinomas of the esophagus, gastrointestinal tract and breast, and hematological malignancies.

In another aspect the present disclosure provides a method of treating an infectious disease in a subject in need thereof, the method comprising administering to the subject a therapeutically effective amount of a compound of formula (I), or a pharmaceutically acceptable salt thereof. In a first aspect of the fourth aspect the infectious disease is caused by a virus. In a second aspect the virus is selected from HIV, Hepatitis A, Hepatitis B, Hepatitis C, herpes viruses, and influenza.

In another aspect the present disclosure provides a method of treating septic shock in a subject in need thereof, the method comprising administering to the subject a therapeutically effective amount of a compound of formula (I), or a pharmaceutically acceptable salt thereof.

An additional aspect of the present disclosure is directed to a method of blocking the interaction of PD-1 with PD-L1 in a subject, wherein the method comprises administering to the subject a therapeutically effective amount of a compound of Formula (I), or a pharmaceutically acceptable salt thereof.

Administration of a therapeutic agent described herein includes, without limitation, administration of a therapeutically effective amount of therapeutic agent. The term “therapeutically effective amount” as used herein refers, without limitation, to an amount of a therapeutic agent to treat a condition treatable by administration of a composition comprising the PD-1/PD-L1 binding inhibitors described herein. That amount is the amount sufficient to exhibit a detectable therapeutic or ameliorative effect. The effect can include, for example and without limitation, treatment of the conditions listed herein. The precise effective amount for a subject will depend upon the subject's size and health, the nature and extent of the condition being treated, recommendations of the treating physician, and therapeutics or combination of therapeutics selected for administration.

For administration of the macrocycles described herein, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg.

An additional aspect of the subject matter described herein is the use of the disclosed compounds as radiolabeled ligands for development of ligand binding assays or for monitoring of in vivo adsorption, metabolism, distribution, receptor binding or occupancy, or compound disposition. For example, a macrocyclic compound described herein can be prepared using a radioactive isotope and the resulting radiolabeled compound can be used to develop a binding assay or for metabolism studies. Alternatively, and for the same purpose, a macrocyclic compound described herein can be converted to a radiolabeled form by catalytic tritiation using methods known to those skilled in the art.

The macrocyclic compounds of the present disclosure can also be used as PET imaging agents by adding a radioactive tracer using methods known to those skilled in the art.

### Pharmaceutical Compositions

Another aspect of the present disclosure is directed to a composition, e.g., a pharmaceutical composition, containing one or a combination of the compounds described within the present disclosure, Formulated together with a pharmaceutically acceptable carrier. Pharmaceutical compositions of the disclosure also can be administered in combination therapy, i.e., combined with other agents. For example, the combination therapy can include a macrocyclic compound combined with at least one other anti-inflammatory or immunosuppressant agent. Examples of therapeutic agents that can be used in combination therapy are described in greater detail below in the section on uses of the compounds of the disclosure.

As used herein, “pharmaceutically acceptable carrier” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound can be coated in a material to protect the compound from the action of acids and other natural conditions that can inactivate the compound.

A pharmaceutical composition of the disclosure also can include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutical compositions of the present disclosure can be administered via one or more routes of administration using one or more of a variety of methods known in the art. As will be appreciated by the skilled artisan, the route and/or mode of administration will vary depending upon the desired results. In some aspects, the routes of administration for macrocyclic compounds of the disclosure include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, some methods of preparation are vacuum drying and freeze-drying (lyophilization) that yield a powder of the active ingredient plus any additional desired ingredient from a previously sterile-filtered solution thereof.

Examples of suitable aqueous and non-aqueous carriers that can be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions can also contain adjuvants such as preservatives, wetting agents, emulsifying agents and dispersing agents. Prevention of presence of microorganisms can be ensured both by sterilization procedures, supra, and by the inclusion of various antibacterial and antifungal agents, for example, paraben, chlorobutanol, phenol sorbic acid, and the like. It can also be desirable to include isotonic agents, such as sugars, sodium chloride, and the like into the compositions. In addition, prolonged absorption of the injectable pharmaceutical form can be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. Supplementary active compounds can also be incorporated into the compositions.

Therapeutic compositions typically must be sterile and stable under the conditions of manufacture and storage. The composition can be Formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be desirable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Alternatively, the compounds of the disclosure can be administered via a non-parenteral route, such as a topical, epidermal or mucosal route of administration, for example, intranasally, orally, vaginally, rectally, sublingually or topically.

Any pharmaceutical composition contemplated herein can, for example, be delivered orally via any acceptable and suitable oral preparation. Exemplary oral preparations include, but are not limited to, for example, tablets, troches, lozenges, aqueous and oily suspensions, dispersible powders or granules, emulsions, hard and soft capsules, liquid capsules, syrups, and elixirs. Pharmaceutical compositions intended for oral administration can be prepared according to any methods known in the art for manufacturing pharmaceutical compositions intended for oral administration. In order to provide pharmaceutically palatable preparations, a pharmaceutical composition in accordance with the disclosure can contain at least one agent selected from sweetening agents, flavoring agents, coloring agents, demulcents, antioxidants, and preserving agents.

A tablet can, for example, be prepared by admixing at least one compound of Formula (I) and/or at least one pharmaceutically acceptable salt thereof with at least one nontoxic pharmaceutically acceptable excipient suitable for the manufacture of tablets. Exemplary excipients include, but are not limited to, for example, inert diluents, such as, for example, calcium carbonate, sodium carbonate, lactose, calcium phosphate, and sodium phosphate; granulating and disintegrating agents, such as, for example, microcrystalline cellulose, sodium crosscarmellose, corn starch, and alginic acid; binding agents such as, for example, starch, gelatin, polyvinyl-pyrrolidone, and acacia; and lubricating agents, such as, for example, magnesium stearate, stearic acid, and talc. Additionally, a tablet can either be uncoated, or coated by known techniques to either mask the bad taste of an unpleasant tasting drug, or delay disintegration and absorption of the active ingredient in the gastrointestinal tract thereby sustaining the effects of the active ingredient for a longer period. Exemplary water soluble taste masking materials include, but are not limited to, hydroxypropyl-methylcellulose and hydroxypropyl-cellulose. Exemplary time delay materials include, but are not limited to, ethyl cellulose and cellulose acetate butyrate.

Hard gelatin capsules can, for example, be prepared by mixing at least one compound of Formula (I) and/or at least one salt thereof with at least one inert solid diluent, such as, for example, calcium carbonate; calcium phosphate; and kaolin.

Soft gelatin capsules can, for example, be prepared by mixing at least one compound of Formula (I) and/or at least one pharmaceutically acceptable salt thereof with at least one water soluble carrier, such as, for example, polyethylene glycol; and at least one oil medium, such as, for example, peanut oil, liquid paraffin, and olive oil.

An aqueous suspension can be prepared, for example, by admixing at least one compound of Formula (I) and/or at least one pharmaceutically acceptable salt thereof with at least one excipient suitable for the manufacture of an aqueous suspension, include, but are not limited to, for example, suspending agents, such as, for example, sodium carboxymethylcellulose, methylcellulose, hydroxypropylmethyl-cellulose, sodium alginate, alginic acid, polyvinyl-pyrrolidone, gum tragacanth, and gum acacia; dispersing or wetting agents, such as, for example, a naturally-occurring phosphatide, e.g., lecithin; condensation products of alkylene oxide with fatty acids, such as, for example, polyoxyethylene stearate; condensation products of ethylene oxide with long chain aliphatic alcohols, such as, for example, heptadecathylene-oxycetanol; condensation products of ethylene oxide with partial esters derived from fatty acids and hexitol, such as, for example, polyoxyethylene sorbitol monooleate; and condensation products of ethylene oxide with partial esters derived from fatty acids and hexitol anhydrides, such as, for example, polyethylene sorbitan monooleate. An aqueous suspension can also contain at least one preservative, such as, for example, ethyl and n-propyl p-hydroxybenzoate; at least one coloring agent; at least one flavoring agent; and/or at least one sweetening agent, including but not limited to, for example, sucrose, saccharin, and aspartame.

Oily suspensions can, for example, be prepared by suspending at least one compound of Formula (I) and/or at least one pharmaceutically acceptable salt thereof in either a vegetable oil, such as, for example, arachis oil, sesame oil, and coconut oil; or in mineral oil, such as, for example, liquid paraffin. An oily suspension can also contain at least one thickening agent, such as, for example, beeswax, hard paraffin, and cetyl alcohol. In order to provide a palatable oily suspension, at least one of the sweetening agents already described herein above, and/or at least one flavoring agent can be added to the oily suspension. An oily suspension can further contain at least one preservative, including, but not limited to, for example, an anti-oxidant, such as, for example, butylated hydroxyanisol, and alpha-tocopherol.

Dispersible powders and granules can, for example, be prepared by admixing at least one compound of Formula (I) and/or at least one pharmaceutically acceptable salt thereof with at least one dispersing and/or wetting agent, at least one suspending agent, and/or at least one preservative. Suitable dispersing agents, wetting agents, and suspending agents are already described above. Exemplary preservatives include, but are not limited to, for example, antioxidants, e.g., ascorbic acid. In addition, dispersible powders and granules can also contain at least one excipient, including, but not limited to, for example, sweetening agents, flavoring agents, and coloring agents.

An emulsion of at least one compound of Formula (I) and/or at least one pharmaceutically acceptable salt thereof can, for example, be prepared as an oil-in-water emulsion. The oily phase of the emulsions comprising the compounds of Formula (I) can be constituted from known ingredients in a known manner. The oil phase can be provided by, but is not limited to, for example, a vegetable oil, such as, for example, olive oil and arachis oil; a mineral oil, such as, for example, liquid paraffin; and mixtures thereof. While the phase can comprise merely an emulsifier, it can comprise a mixture of at least none emulsifier with a fat or an oil or with both a fat and an oil. Suitable emulsifying agents include, but are not limited to, for example, naturally-occurring phosphatides, e.g., soy bean lecithin, esters or partial esters derived from fatty acids and hexitol anhydrides, such as, for example sorbitan monoleate, and condensation products of partial esters with ethylene oxide, such as, for example, polyoxyethylene sorbitan monooleate. In some aspects, a hydrophilic emulsifier is included together with a lipophilic emulsifier which acts as a stabilizer. It is also sometimes desirable to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream Formulations. An emulsion can also contain a sweetening agent, a flavoring agent, a preservative, and/or an antioxidant. Emulsifiers and emulsion stabilizers suitable for use in the Formulation of the present disclosure include Tween 60, Span 80, cetostearyl alcohol, myristyl alcohol, glyceryl monostearate, sodium lauryl sulfate, glyceral disterate alone or with a wax, or other materials well known in the art.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such Formulations are patented or generally known to those skilled in the art. See, e.g., Robinson, J. R., ed., Sustained and Controlled Release Drug Delivery Systems, Marcel Dekker, Inc., New York (1978).

Therapeutic compositions can be administered with medical devices known in the art. For example, in one aspect, a therapeutic composition of the disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medication through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain aspects, the compounds of the disclosure can be formulated to ensure proper distribution in vivo. For example, the blood-brain barrier (BBB) excludes many highly hydrophilic compounds. To ensure that therapeutic compounds of the disclosure cross the BBB (if desired), they can be Formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., Ranade, V. V., J. Clin. Pharmacol., 29:685 (1989)). Exemplary targeting moieties include folate or biotin (see, e.g., U.S. Pat. No. 5,416,016 to Low et al.); mannosides (Umezawa et al., Biochem. Biophys. Res. Commun., 153:1038 (1988)); macrocyclic compounds (Bloeman, P. G. et al., FEBS Lett., 357:140 (1995); Owais, M. et al., Antimicrob. Agents Chemother., 39:180 (1995)); surfactant protein A receptor (Briscoe et al., Am. J. Physiol., 1233:134 (1995)); p120 (Schreier et al., J. Biol. Chem., 269:9090 (1994)); see also Keinanen, K. et al., FEBS Lett., 346:123 (1994); Killion, J. J. et al., Immunomethods 4:273 (1994).

In certain aspects, the compounds of the present disclosure can be administered parenterally, i.e., by injection, including, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and/or infusion.

In some aspects, the compounds of the present disclosure can be administered orally, i.e, via a gelatin capsule, tablet, hard or soft capsule, or a liquid capsule. The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

### EXAMPLES

The following examples are included to demonstrate various aspics of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific examples which are disclosed and still obtain a like or similar result without departing from the spirit and scope of the disclosure.

The compounds can be made by methods known in the art including those described below and including variations within the skill of the art. Some reagents and intermediates are known in the art. Other reagents and intermediates can be made by methods known in the art using readily available materials. Any variables (e.g. numbered “R” substituents) used to describe the synthesis of the compounds are intended only to illustrate how to make the compounds and are not to be confused with variables used in the claims or in other sections of the specification. The following methods are for illustrative purposes and are not intended to limit the scope of the disclosure.

Abbreviations used in the schemes generally follow conventions used in the art. Chemical abbreviations used in the specification and examples are defined as follows: Ph=phenyl; Bn=benzyl; i-Bu=iso-butyl; i-Pr=iso-propyl; Me=methyl; Et=ethyl; Pr=n-propyl; Bu=n-butyl; t-Bu=tert-butyl; Trt=trityl; TMS=trimethylsilyl; TIS=triisopropylsilane; Et₂O=diethyl ether; HOAc or AcOH=acetic acid; MeCN or AcCN=acetonitrile; DMF=N,N-dimethylformamide; EtOAc=ethyl acetate; THF=tetrahydrofuran; TFA=trifluoroacetic acid; TFE=α,α,α-trifluoroethanol; Et₂NH=diethylamine; NMN=N-methylmorpholine; NMP=N-methylpyrrolidone; DCM=dichloromethane; TEA=trimethylamine; min.=minute(s); h or hr=hour(s); L=liter; mL or ml=milliliter; μL=microliter; g=gram(s); mg=milligram(s); mol=mole(s); mmol=millimole(s); meq=milliequivalent; rt or RT=room temperature; sat or sat'd=saturated; aq.=aqueous; mp=melting point; FMOC for fluorenylmethoxycarbonyl; HOBt for 1-hydroxybenzotriazole hydrate; HOAT for 1-hydroxy-7-azabenzotriazole; DIC for diisopropylcarbodiimide; HBTU for 2-(1H-benzotriazol-1-yl)-1,1,3,3-tetramethyluronium hexafluorophosphate, hexafluorophosphate benzotriazole tetramethyl uronium; BOP for benzotriazol-1-yloxytris(dimethylamino)phosphonium hexafluorophosphate; PyBOP for benzotriazol-1-yl-oxytripyrrolidinophosphonium hexafluorophosphate; HCTU for HCTU for 1-[bis(dimethylamino)methylen]-5-chlorobenzotriazolium 3-oxide hexafluorophosphate or N,N,N′,N′-tetramethyl-O-(6-chloro-1H-benzotriazol-1-yl)uronium hexafluorophosphate; HATU for 1-[bis(dimethylamino)methylene]-1H-1,2,3-triazolo[4,5-b]pyridinium 3-oxid hexafluorophosphate or N-[(dimethylamino)-1H-1,2,3-triazolo-[4,5-b]pyridin-1-ylmethylene]-N-methylmethanaminium hexafluorophosphate N-oxide; iPrNEt₂ or DIPEA or DIEA for diisopropylethylamine; DTT for dithiothreitol (Cleland's reagent); TCEP for tris-2(-carboxyethyl)-phosphine; DMSO for dimethylsulfoxide; CAN for ceric ammonium nitrate; DVB for divinylbenzene; Pbf for 2,2,4,6,7-Pentamethyldihydrobenzofuran-5-sulfonyl chloride; Trt for trityl; t-Bu for tert-butyl; BOC for tert-butoxycarbonyl; Me for methyl; NMM for N-methylmorpholine; rt or RT for room temperature or retention time (context will dictate); min or mins for minutes; h or hr or hrs for hours; NOS-CL for 4-nitrobenzenesulfonyl chloride; DBU for 1,8-diazabicyclo[5.4.0]undec-7-ene; dtbpf for [1,1′-bis(di-tert-butylphosphino)ferrocene]; MeOH for methanol; Fmoc-OSu for N-(9-Fluorenylmethoxy carbonyloxy)succiniimide, 9-fluorenylmethyl-succinimidyl carbonate; Ac for acetyl; SPhos for 2-dicyclohexylphosphino-2′,6′-dirnethoxybiphenyl; dba for tris(dibenzylideneacetone); TMS for trimethylsilyl; Hex for hexyl; XPhos for 2-dicyclohexylphosphino-2′,4′,6′-triisopropylbiphenyl; TEMPO for (2,2,6,6-tetramethylpiperidin-1-yl)oxyl or (2,2,6,6-tetramethylpiperidin-1-yl)oxidanyl; ACN or MeCN for acetonitrile; EA or EtOAc for ethyl acetate; Et₃N or TEA for trimethylamine; PE for petroleum ether; KHMDS for potassium hexamethyldisilazide; HFIP for hexafluoroisopropanol; TCNHPI for N-hydroxytetrachlorophthalinide; DIAD for diisopropyl azodicarboxylate; DtBuPf for 1,1′-Bis(di-tert-butylphosphino)ferrocene; and t-Bu for tert-butyl; HPLC=high performance liquid chromatography; LC/MS=high performance liquid chromatography/mass spectrometry; MS or Mass Spec=mass spectrometry; NMR=nuclear magnetic resonance; Sc or SC or SQ=subcutaneous; and IP or ip=intra-peritoneal

### Example 1: General Synthetic Procedures and Analytical Methods

The macrocyclic compounds of the present disclosure can be produced by methods known in the art, such as they can be synthesized chemically, recombinantly in a cell free system, recombinantly within a cell or can be isolated from a biological source. Chemical synthesis of a macrocyclic compound of the present disclosure can be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. A preferred method to synthesize the macrocyclic compounds and analogs thereof described herein is chemical synthesis using various solid-phase techniques such as those described in Chan, W. C. et al, eds., Fmoc Solid Phase Synthesis, Oxford University Press, Oxford (2000); Barany, G. et al, The Peptides: Analysis, Synthesis, Biology, Vol. 2: “Special Methods in Peptide Synthesis, Part A”, pp. 3-284, Gross, E. et al, eds., Academic Press, New York (1980); in Atherton, E., Sheppard, R. C. Solid Phase Peptide Synthesis: A Practical Approach, IRL Press, Oxford, England (1989); and in Stewart, J. M. Young, J. D. Solid-Phase Peptide Synthesis, 2nd Edition, Pierce Chemical Co., Rockford, IL (1984). The preferred strategy is based on the (9-fluorenylmethyloxycarbonyl) group (Fmoc) for temporary protection of the α-amino group, in combination with the tert-butyl group (tBu) for temporary protection of the amino acid side chains (see for example Atherton, E. et al, “The Fluorenylmethoxycarbonyl Amino Protecting Group”, in The Peptides: Analysis, Synthesis, Biology, Vol. 9: “Special Methods in Peptide Synthesis, Part C”, pp. 1-38, Undenfriend, S. et al, eds., Academic Press, San Diego (1987).

The compounds can be synthesized in a stepwise manner on an insoluble polymer support (also referred to as “resin”) starting from the C-terminus of the peptide. A synthesis is begun by appending the C-terminal amino acid of the compound to the resin through formation of an amide or ester linkage. This allows the eventual release of the resulting peptide as a C— terminal amide or carboxylic acid, respectively.

The C-terminal amino acid and all other amino acids used in the synthesis are required to have their α-amino groups and side chain functionalities (if present) differentially protected such that the α-amino protecting group may be selectively removed during the synthesis. The coupling of an amino acid is performed by activation of its carboxyl group as an active ester and reaction thereof with the unblocked α-amino group of the N-terminal amino acid appended to the resin. The sequence of α-amino group deprotection and coupling is repeated until the entire sequence is assembled. The compound is then released from the resin with concomitant deprotection of the side chain functionalities, usually in the presence of appropriate scavengers to limit side reactions. The resulting compound is finally purified by reverse phase HPLC.

The synthesis of the peptidyl-resins required as precursors to the final compounds utilizes commercially available cross-linked polystyrene polymer resins (Novabiochem, San Diego, CA; Applied Biosystems, Foster City, CA). Preferred solid supports are: 4-(2′,4′-dimethoxyphenyl-Fmoc-aminomethyl)-phenoxyacetyl-p-methyl benzhydrylamine resin (Rink amide MBHA resin); 9-Fmoc-amino-xanthen-3-yloxy-Merrifield resin (Sieber amide resin); 4-(9-Fmoc)aminomethyl-3,5-dimethoxyphenoxy)valerylaminomethyl-Merrifield resin (PAL resin), for C-terminal carboxamides. Coupling of first and subsequent amino acids can be accomplished using HOBt, 6-Cl-HOBt or HOAt active esters produced from DIC/HOBt, HBTU/HOBt, BOP, PyBOP, or from DIC/6-Cl-HOBt, HCTU, DIC/HOAt or HATU, respectively. Preferred solid supports are: 2-chlorotrityl chloride resin and 9-Fmoc-amino-xanthen-3-yloxy-Merrifield resin (Sieber amide resin) for protected peptide fragments. Loading of the first amino acid onto the 2-chlorotrityl chloride resin is best achieved by reacting the Fmoc-protected amino acid with the resin in dichloromethane and DIEA. If necessary, a small amount of DMF may be added to solubilize the amino acid.

The syntheses of the compounds described herein can be carried out by using a single or multi-channel peptide synthesizer, such as an CEM Liberty Microwave synthesizer, or a Protein Technologies, Inc. Prelude (6 channels) or Symphony (12 channels) or Symphony X (24 channels) synthesizer.

Useful Fmoc amino acids derivatives are shown in Table 1.

TABLE 1
Examples of Orthogonally Protected Amino Acids
used in Solid Phase Synthesis

The peptidyl-resin precursors for their respective compounds may be cleaved and deprotected using any standard procedure (see, for example, King, D. S. et al, Int. J. Peptide Protein Res., 36:255-266 (1990)). A desired method is the use of TFA in the presence of TIS as scavenger and DTT or TCEP as the disulfide reducing agent. Typically, the peptidyl-resin is stirred in TFA/TIS/DTT (95:5:1 to 97:3:1), v:v:w; 1-3 mL/100 mg of peptidyl resin) for 1.5-3 hrs at room temperature. The spent resin is then filtered off and the TFA solution was cooled and Et₂O solution was added. The precipitates were collected by centrifuging and decanting the ether layer (3×). The resulting crude compound is either redissolved directly into DMF or DMSO or CH₃CN/H₂O for purification by preparative HPLC or used directly in the next step.

Compounds with the desired purity can be obtained by purification using preparative HPLC, for example, on a Waters Model 4000 or a Shimadzu Model LC-8A liquid chromatography. The solution of crude compound is injected into a YMC S5 ODS (20×100 mm) column and eluted with a linear gradient of MeCN in water, both buffered with 0.1% TFA, using a flow rate of 14-20 mL/min with effluent monitoring by UV absorbance at 217 or 220 nm. The structures of the purified compound can be confirmed by electro-spray MS analysis.

List of unnatural amino acids referred to herein is provided in Table 2.

```
TABLE 2
Examples of Orthogonally Protected Amino Acids
used in Solid Phase Synthesis
  Fmoc-Phe(2,5-di-Me)-OH
  Fmoc-Phe(4-OCF₃)-OH
  Fmoc-4-Pya(2-OMe)-OH
  Fmoc-3-Pya(6-o-tolyl)-OH (S)-2-((((9H-fluoren-9- yl)methoxy)carbonyl)amino)-3-(6- (o-tolyl)pyridin-3-yl)propanoic acid
  Fmoc-Bip(4′-COOtBu)-OH
  Fmoc-(S)-β-Me-Trp(Boc)-OH
  Fmoc-Bip(3′-COOtBu)-OH
  Fmoc-Phe(4-COOtBu)
  Fmoc-Trp(CH₂COOtBu)-OH
  Fmoc-Tyr(CH₂COOtBu)-OH
  Fmoc-Ala(β-morpholin-4-yl)-OH(2S)-2- ({[(9H-fluoren-9-yl)methoxy]carbonyl}amino)- 3-(morpholin-4-yl)propanoic acid
  (S)-2-Fmoc-amino--2- (1-Boc-aminomethyl-cyclopropyl)acetic acid
  (S)-2-Fmoc-amino-2- (1-Boc-azetidin-3-yl)acetic acid
  Fmoc-β,β-diMe-Glu(tBu)-OH
  Fmoc-β,-β-diMe-Orn(Boc)-OH
  Fmoc-Phe(3-Me-4-F)-OH
  Fmoc-Phe(3-COOtBu)-OH
  Fmoc-Phe(2,4-di-F-5-OMe)-OH
  Fmoc-β,β-DiMe-Asp(tBu)-OH
  Fmoc-β-Ala-OH
  Fmoc-HomoLeu-OH
  Fmoc-Ile-OH
  Fmoc-HomoCha-OH
  Fmoc-Cha-OH
  (Fmoc-Aib-OH)
  Fmoc-Nle-OH
  Fmoc-tBu-Gly-OH (Fmoc-Tle-OH)
  Fmoc-Abu-OH
  Fmoc-Cyclopropyl-Gly-OH
  (Fmoc-Pra-OH)
  1-(Fmoc- amino)cyclopropane carboxylic acid
  Fmoc-1-amino-3- OtBu-cyclopentane- 1-carboxylic acid
  Fmoc-1- aminocyclopentane- 1-carboxylic acid
  Fmoc-cis-Hyp(tBu)-OH
  Fmoc-Hyp(tBu)-OH
  Fmoc-cis-Hyp(Bn)-OH
  Fmoc-NPh-AzaPro-OH
  Fmoc-Pip-OH
  Fmoc-Chg-OH
  Fmoc-Ala(β-cyclopropyl)-OH Fmoc-Cyclopropyl-Ala-OH
  Fmoc-Nva-OH
  Fmoc-Cit-OH
  Fmoc-trans-4-NHBoc-Pro-OH
  Fmoc-cis-4-Ph-Pro-OH
  Fmoc-4-Ph-Pro-OH
  Fmoc-azetidine-2- carboxylic acid Fmoc-Azt-OH
  Fmoc-isoPro(Boc)-OH
  Fmoc-homoGlu(OtBu)-OH
  Fmoc-Orn(Boc)-OH
  Fmoc-Dab(Boc)-OH
  Fmoc-Dap(Boc)-OH
  1-Fmoc-4- (tert-butoxycarbonyl)piperazine- 2-carboxylic acid 1-Fmoc-4-Boc-piperazine-OH
  4-N-Fmoc- morpholinecarboxylic acid
  Fmoc-Ala(β-1-Boc-piperidin-3-yl)-OH
  (2S)-2-{1-[(1Z)- {[(tert-butoxy)carbonyl]amino} ({[(tert-butoxy)carbonyl]imino})methyl]piperidin-4-yl}- 2-({[(9H-fluoren-9-yl)methoxy]carbonyl}amino)acetic acid
  Fmoc-Ala(β-1-Boc-piperidin-4-yl)-OH
  Fmoc-Ala(β-isoquinolin-7-yl)-OH
  Fmoc-Ala(β-quinolin-6-yl)-OH
  Fmoc-Ala(β-isoquinolin-3-yl)-OH
  Fmoc-Ala(β-isoquinolin-6-yl)-OH
  Fmoc-Ala(β-isoquinolin-4-yl)-OH
  Fmoc-Trp(1,2-N2,7-Me)-OH
  Fmoc-Phe(3-OCH₂COOtBu)-OH
  Fmoc-AllylGly-OH
  Fmoc-2-furanyl-Ala-OH
  Fmoc-3-thienyl-Ala-OH
  Fmoc-2-thienyl-Ala-OH
  Fmoc-His(3-Me)-OH
  Fmoc-2-thienyl-5-Br-Ala-OH
  Fmoc-Phe(4-Cl)-OH
  Fmoc-Phe(3-Cl)-OH
  Fmoc-Phe(2-Cl)-OH
  Fmoc-Phe(3,5-di-F)-OH
  Fmoc-Phe(3,4-di-Cl)-OH
  Fmoc-Phe(3,4-diF)-OH
  Fmoc-Bpa-OH
  Fmoc-PhenylGly-OH (Fmoc-Phg-OH)
  Fmoc-3-Ph-Phe-OH
  Fmoc-4-Thiazolylalanine Fmoc-Ala(4-Thiazol-3-yl)
  Fmoc-Phe(3,4,5-tri-F)-OH
  Fmoc--Phe(2,3,4,5,6-penta-F)-OH
  Fmoc-His(1-Me)-OH
  Fmoc-Bzt-OH
  Fmoc-3-Py-Ala-OH (Fmoc-3-Pya-OH)
  Fmoc-4-Py-Ala-OH (Fmoc-4-Pya-OH)
  Fmoc-2-Py-Ala-OH (Fmoc-2-Pya-OH)
  Fmoc-1-Nal-OH
  Fmoc-Phe(4-CF₃)-OH
  Fmoc-Phe(3-CF₃)-OH
  Fmoc-Phe(2-CF₃)-OH
  Fmoc-Phe(4-F)-OH
  Fmoc-Phe(3-F)-OH
  Fmoc-Phe(4-I)-OH
  Fmoc-Tyr(Me)-OH (Fmoc-Phe(4-OMe)-OH)
  Fmoc-Phe(-3-OMe)-OH
  Fmoc-Phe(2-OMe)-OH
  Fmoc-Tyr(Bn)-OH
  Fmoc-2-Nal-OH
  Fmoc-β-hydroxy-Phe-OH
  Fmoc-Phe(2-F)-OH
  Fmoc-Tyr(Et)-OH
  Fmoc-Tyr(CH₂CH₂NHBoc)-OH
  Fmoc-Tyr(propargyl)-OH
  Fmoc-Phe(4-NO₂)-OH
  Fmoc-Tyr(tBu)-OH
  Fmoc-homo-homoPhe-OH
  Fmoc-Phe(4-aminomethyl(Boc))-OH
  Fmoc-Phe(4-tBu)-OH
  Fmoc-Phe(4-CN)-OH
  Fmoc-Phe(4-CONH₂)-OH
  Fmoc-Phe(4-COOtBu)--OH
  Fmoc-α-Me-Phe-OH
  Fmoc-Phe(4-OPh)-OH (Fmoc-Tyr(Ph)-OH)
  Fmoc-Phe(4-Me)-OH
  Fmoc-Phe(4-NHBoc)-OH
  Fmoc-Tyr(CH₂COOtBu)-OH
  Fmoc-Tyr(propargyl)-OH
  Fmoc-HomoPhe-OH
  Fmoc-Hom4-Py-Ala-OH
  Fmoc-Phe(3-CN)-OH
  Fmoc-4-Phe(iPr)-OH
  Fmoc-Homo-Ser(Bn)-OH
  Fmoc-Bip-OH
  Fmoc-Bip(2′-Me)-OH
  Fmoc-N-Me-Gly-OH (Fmoc-mGly-OH) N
  Fmoc-N-Me-Nle-OH (Fmoc-mNle-OH)
  Fmoc-N-Me-Asp(OtBu)-OH (Fmoc-mAsp(OtBu)-OH)
  Fmoc-N-Me-Ala-OH (Fmoc-mAla-OH)
  Fmoc-N-Me-Cys(Trt)-OH (Fmoc-mCys(Trt)-OH)
  Fmoc-HomoCys(Trt)-OH
  Fmoc-N-Me-Phe-OH (Fmoc-mPhe-OH)
  Fmoc-Iso-Tic-OH
  Fmoc-Tic-OH
  Fmoc-N-Me-Tyr(OtBu)-OH (Fmoc-mTyr(OtBu)-OH)
  Fmoc-N-Me-Val-OH (Fmoc-mVal-OH)
  Fmoc-Glu-OtBu
  Fmoc-Glu(2-phenylisopropyloxy)-OH
  Fmoc-S-hydroxyvaline Fmoc-V(β-OH)-OH
  Fmoc-Ala(β-Cha(4,4-di-F))-OH
  Fmoc-Dab(COtBu)-OH
  Fmoc-Dab(Ac)-OH
  Fmoc-β,β-dimethyl-Cys(Trt)-OH Fmoc-Pen(Trt)-OH
  2-NHFmoc-2- (oxan-4-yl)acetic acid
  Fmo-Lys(N,N-di-Me)-OH
  FMOC-Lys(trimethyl)-OH
  Fmoc-Orn(CO-cyclopropyl)-OH
  Fmoc-Homo Tyr(OtBu)-OH
  Fmoc-4-Cl-2-Me-phenoxyhomoSer-OH
  Fmoc-Phe(3,4-di-OMe)-OH
  Fmoc-Phe(3-Me)-OH
  Fmoc-5-MeO-Trp(Boc)-OH
  Fmoc-Nme-Trp-OH
  Fmoc-7-Me-Trp(Boc)-OH
  Fmoc-N-AcOH-Trp-OH
  Fmoc-Trp(Boc)-OH
  Fmoc-2-Me-Trp(Boc)-OH
  Fmoc-Iso-Trp(Boc)-OH
  Fmoc-Ala-(β-isoquinolin-4-yl)-OH
  Fmoc-Ala-(β-quinolin-6-yl)-OH
  Fmoc-Ala-(β-quinolin-7-yl)-OH
  Fmoc-Ala-(β-isoquinolin-8-yl)-OH
  Fmoc-Ala(β-indazol-3-yl)-OH
  Fmoc-Trp(Boc)Tic-OH
  Fmoc-5-OH-Trp(Boc)-OH
  Fmoc-Phe(3-Br)-OH
  Fmoc-Bip(4′-Cl)-OH
  Fmoc-Bip(4′-COMe)-OH
  Fmoc-Bip(4′-NHCOMe)-OH
  Fmoc-Bip(4′-CH₂CH₂OCH₂COOtBu)-OH
  Fmoc-Bip(4′-CH₂CH₂CN)-OH
  Fmoc-Bip(4′-F)-OH
  Fmoc-Bip(4′-Pyr)-OH
  Fmoc-Bip(3′-Pyr)-OH
  Fmoc-Bip(3′,5′-F2)-OH
  Fmoc-Bip(3′-OMe)-OH
  Fmoc-Bip(3′-Thio)-OH
  Fmoc-Bip(3′-OH)-OH
  Fmoc-Bip(3′-OEt)-OH
  Fmoc-Bip(3′-Cl)-OH
  Fmoc-Bip(3′-Me)-OH
  Fmoc-Bip(3′-F,5′-OMe)-OH
  Fmoc-Bip(3′-CONH₂)-OH
  Fmoc-Bip(3′-OCF₃)-OH
  Fmoc-Bip(3′-F)-OH
  Fmoc-N-Me-Ser(tBu)-OH (Fmoc-mSer(tBu)-OH)
  Fmoc-N-Me-Asn(Trt)-OH (Fmoc-mAsn(Trt)-OH)
  Fmoc-N-MeTrp(Boc)-OH (Fmoc-mTrp(Boc)-OH)
  Fmoc-N-Me-Arg(Pbf)-OH (Fmoc-mArf(Pbf)-OH)
  Fmoc-Gln(Trt)
  Fmoc-N-Me-His(Trt)
  Fmoc-N-Me-Asp(OtBu)-OH (Fmoc-mAsp(OtBu)-OH)
  Fmoc-N-Me-Lys(Boc)-OH (Fmoc-mLys(Boc)-OH)
  Fmoc-N-Me-Glu(OtBu)-OH (Fmoc-nGlu(OtBu)-OH)
  2-(Fmoc-amino)-3- (1H-indol-4-yl)propanoic acid
  Fmoc-Ala(β-1- carbamimidoylpiperidin- 4-yl)-OH
  (S)-1-Fmoc-4-Boc- piperazine-2-carboxylic acid
  Fmoc-Phe(4-N₃)-OH
  Fmoc-α-Me-Glu(tBu)
  Fmoc-β-COOtBu-Glu(tBu)
  Fmoc-Phe(2-OMe-5-Me)-OH
  Fmoc-Tyr(tBu, 2,6-di-F)-OH
```

### Analytical Data:

Mass Spectrometry: “ESI-MS(+)” signifies electrospray ionization mass spectrometry performed in positive ion mode; “ESI-MS(−)” signifies electrospray ionization mass spectrometry performed in negative ion mode; “ESI-HRMS(+)” signifies high-resolution electrospray ionization mass spectrometry performed in positive ion mode; “ESI-HRMS(−)” signifies high-resolution electrospray ionization mass spectrometry performed in negative ion mode. The detected masses are reported following the “m z” unit designation. Compounds with exact masses greater than 1000 were often detected as double-charged or triple-charged ions.

The crude material was purified via preparative LC/MS. Fractions containing the desired product were combined and dried via centrifugal evaporation.

### Analytical LC/MS Condition A:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 50° C.; Gradient: 0-100% B over 3 minutes, then a 0.75-minute hold at 100% B; Flow: 1.0 mL/min; Detection: UV at 220 nm.

### Analytical LC/MS Condition B:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 50° C.; Gradient: 0-100% B over 3 minutes, then a 0.75-minute hold at 100% B; Flow: 1.0 mL/min; Detection: UV at 220 nm.

### Analytical LC/MS Condition C:

Column: Waters Acquity UPLC BEH C18, 2.1×50 mm, 1.7-μm particles; Mobile Phase A: 5:95 acetonitrile:water with 10 mM ammonium acetate; Mobile Phase B: 95:5 acetonitrile:water with 10 mM ammonium acetate; Temperature: 70° C.; Gradient: 0-100% B over 3 minutes, then a 2.0-minute hold at 100% B; Flow: 0.75 mL/min; Detection: UV at 220 nm.

### Analytical LCMS Condition D:

Column: Waters Acquity UPLC BEH C18, 2.1×50 mm, 1.7-μm particles; Mobile Phase A: 5:95 acetonitrile:water with 0.1% trifluoroacetic acid; Mobile Phase B: 95:5 acetonitrile:water with 0.1% trifluoroacetic acid; Temperature: 70° C.; Gradient: 0-100% B over 3 minutes, then a 2.0-minute hold at 100% B; Flow: 0.75 mL/min; Detection: UV at 220 nm.

### Analytical LCMS Condition E:

Column: Kinetex XB C18, 3.0×75 mm, 2.6-μm particles; Mobile Phase A: 10 mM ammonium formate in water:acetonitrile (98:2); Mobile Phase B: 10 mM ammonium formate in Water:acetonitrile (02:98); Gradient: 20-100% B over 4 minutes, then a 0.6-minute hold at 100% B; Flow: 1.0 mL/min; Detection: UV at 254 nm.

### Analytical LCMS Condition F:

Column: Ascentis Express C18, 2.1×50 mm, 2.7-μm particles; Mobile Phase A: 10 mM ammonium acetate in water:acetonitrile (95:5); Mobile Phase B: 10 mM ammonium acetate in Water:acetonitrile (05:95), Temperature: 50° C.; Gradient: 0-100% B over 3 minutes; Flow: 1.0 mL/min; Detection: UV at 220 nm.

### Analytical LCMS Condition G:

Column: X Bridge C18, 4.6×50 mm, 5-μm particles; Mobile Phase A: 0.1% TFA in water; Mobile Phase B: acetonitrile, Temperature: 35° C.; Gradient: 5-95% B over 4 minutes; Flow: 4.0 mL/min; Detection: UV at 220 nm.

### Analytical LCMS Condition H:

Column: X Bridge C18, 4.6×50 mm, 5-μm particles; Mobile Phase A: 10 mM NH₄OAc; Mobile Phase B: methanol, Temperature: 35° C.; Gradient: 5-95% B over 4 minutes; Flow: 4.0 mL/min; Detection: UV at 220 nm.

### Analytical LCMS Condition I:

Column: X Bridge C18, 4.6×50 mm, 5-μm particles; Mobile Phase A: 10 mM NH₄OAc; Mobile Phase B: acetonitrile, Temperature: 35° C.; Gradient: 5-95% B over 4 minutes; Flow: 4.0 mL/min; Detection: UV at 220 nm.

### Analytical LCMS Condition J.

Column: Waters Acquity UPLC BEH C18, 2.1×50 mm, 1.7-μm particles; Mobile Phase A: 5:95 acetonitrile:water with 0.05% trifluoroacetic acid; Mobile Phase B: 95:5 acetonitrile:water with 0.05% trifluoroacetic acid; Temperature: 70° C.; Gradient: 0-100% B over 1.5 minutes, then a 2.0-minute hold at 100% B; Flow: 0.75 mL/min; Detection: UV at 254 nm.

### Analytical LC/MS Condition K:

Column: Waters Acquity UPLC BEH C18, 2.1×50 mm, 1.7-μm particles; Mobile Phase A: 100% water with 0.05% trifluoroacetic acid; Mobile Phase B: 100% acetonitrile with 0.05% trifluoroacetic acid; Temperature: 50° C.; Gradient: 2-98% B over 1.0 minutes, then at 1.0-1.5 minute hold at 100% B; Flow: 0.80 mL/min; Detection: UV at 220 nm.

### Analytical LC/MS Condition L:

Column: Waters Acquity UPLC BEH C18, 2.1×50 mm, 1.7-μm particles; Buffer:10 mM Ammonium Acetate. Mobile Phase A: buffer” CH₃CN (95/5); Mobile Phase B: Mobile Phase B: Buffer:ACN(5:95); Temperature: 50° C.; Gradient: 20-98% B over 2.0 minutes, then at 0.2 minute hold at 100% B; Flow: 0.70 mL/min; Detection: UV at 220 nm.

### Analytical LC/MS Condition M:

Column: Waters Acquity UPLC BEH C18, 3.0×50 mm, 1.7-μm particles; Mobile Phase A: 95% water and 5% water with 0.1% trifluoroacetic acid; Mobile Phase B: 95% acetonitrile and 5% water with 0.1% trifluoroacetic acid; Temperature: 50° C.; Gradient: 20-100% B over 2.0 minutes, then at 2.0-2.3 minute hold at 100% B; Flow: 0.7 mL/min; Detection: UV at 220 nm.

### Prelude Method:

All manipulations were performed under automation on a Prelude peptide synthesizer (Protein Technologies). Unless noted, all procedures were performed in a 45-mL polypropylene reaction vessel fitted with a bottom frit. The reaction vessel connects to the Prelude peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution was used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis: Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Prelude Method” describe an experiment performed on a 0.100 mmol scale, where the scale is determined by the amount of Sieber or Rink or 2-chlorotrityl or PL-FMP resin. This scale corresponds to approximately 140 mg of the Sieber amide resin described above. All procedures can be scaled down from the 0.100 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- used the “Double-coupling procedure” described below.

### Resin-Swelling Procedure:

To a 45-mL polypropylene solid-phase reaction vessel was added Sieber amide resin (140 mg, 0.100 mmol). The resin was washed (swelled) two times as follows: to the reaction vessel was added DMF (5.0 mL) through the top of the vessel “DMF top wash” upon which the mixture was periodically agitated for 10 minutes before the solvent was drained through the frit.

### Single-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 60-120 minutes, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Double-Coupling Procedure:

To the reaction vessel containing the resin from the previous step was added piperidine:DMF (20:80 v/v, 5.0 mL). The mixture was periodically agitated for 5.0 minutes and then the solution was drained through the frit. To the reaction vessel was added piperidine:DMF (20:80 v/v, 5.0 mL). The mixture was periodically agitated for 5.0 minutes and then the solution was drained through the frit. The resin was washed successively six times as follows: for each wash, DMF (6.0 mL) was added through the top of the vessel and the resulting mixture was periodically agitated for 1.0 minutes before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hour, then the reaction solution was drained through the frit. The resin was washed successively two times as follows: for each wash, DMF (5.0 mL) was added through the top of the vessel and the resulting mixture was periodically agitated for 1.0 minute before the solution was drained through the frit. To the reaction vessel was added the amino acid (0.2 M in DMF, 5.0 mL, 10 equiv), then HATU (0.4 M in DMF, 2.5 mL, 10 equiv), and finally NMM (0.8 M in DMF, 2.5 mL, 20 equiv). The mixture was periodically agitated for 1-1.5 hours, then the reaction solution was drained through the frit. The resin was washed successively four times as follows: for each wash, DMF (5.0 mL) was added through the top of the vessel and the resulting mixture was periodically agitated for 1.0 minute before the solution was drained through the frit. The resulting resin was used directly in the next step.

### Single-Coupling Manual Addition Procedure A:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-2 mL) was added manually using a pipette from the top of the vessel while the bottom of the vessel remained attached to the instrument, then the vessel was closed. The automatic program was resumed and HATU (0.4 M in DMF, 1.3 mL, 4 equiv) and NMM (1.3 M in DMF, 1.0 mL, 8 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, followed by the manual addition of HATU (2-4 equiv, same equiv as the unnatural amino acid), and then the vessel was closed. The automatic program was resumed and NMM (1.3 M in DMF, 1.0 mL, 8 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 5.0 mL). The mixture was periodically agitated for 5 minutes and then the solution was drained through the frit. To the reaction vessel was added piperidine:DMF (20:80 v/v, 5.0 mL). The mixture was periodically agitated for 5 minutes and then the solution was drained through the frit. The resin was washed successively six times as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed twice as follows: for each wash, DMF (6.0 mL) was added through the top of the vessel and the resulting mixture was periodically agitated for one minute before the solution was drained through the frit. To the reaction vessel was added the chloroacetic anhydride solution (0.4 M in DMF, 5.0 mL, 20 equiv), then N-methylmorpholine (0.8 M in DMF, 5.0 mL, 40 equiv). The mixture was periodically agitated for 15 minutes, then the reaction solution was drained through the frit. The resin was washed successively five times as follows: for each wash, DMF (6.0 mL) was added through the top of the vessel and the resulting mixture was periodically agitated for one minute before the solution was drained through the frit. The resin was washed successively four times as follows: for each wash, DCM (6.0 mL) was added through the top of the vessel and the resulting mixture was periodically agitated for one minute before the solution was drained through the frit. The resin was then dried with nitrogen flow for 10 minutes. The resulting resin was used directly in the next step.

### Symphony Method:

All manipulations were performed under automation on a 12-channel Symphony peptide synthesizer (Protein Technologies). Unless noted, all procedures were performed in a 25-mL polypropylene reaction vessel fitted with a bottom frit. The reaction vessel connects to the Symphony peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. Amino acid solutions were generally not used beyond two weeks from preparation. HATU solution were used within 7-14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading.

PL-FMP resin: (4-Formyl-3-methoxyphenoxymethyl)polystyrene.

Fmoc-glycine-2-chlorotrityl chloride resin, 200-400 mesh, 1% DVB, 0.63 mmol/g loading.

Common amino acids used are listed below with side-chain protecting groups indicated inside parenthesis: Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony Method” describe an experiment performed on a 0.05 mmol scale, where the scale is determined by the amount of Sieber or Rink or chlorotrityl linker or PL-FMP bound to the resin. This scale corresponds to approximately 70 mg of the Sieber resin described above. All procedures can be scaled up from the 0.05 mmol scale by adjusting the described volumes by the multiple of the scale.

Prior to the amino acid coupling, all peptide synthesis sequences began with a resin-swelling procedure, described below as “Resin-swelling procedure”. Coupling of amino acids to a primary amine N-terminus used the “Single-coupling procedure” described below.

### Resin-Swelling Procedure:

To a 25-mL polypropylene solid-phase reaction vessel was added Sieber resin (70 mg, 0.05 mmol). The resin was washed (swelled) as follows: to the reaction vessel was added DMF (2.0 mL), upon which the mixture was periodically agitated for 10 minutes before the solvent was drained through the frit.

### Single-Coupling Procedure:

To the reaction vessel containing the resin from the previous step was added DMF (2.5 mL) three times, upon which the mixture was agitated for 30 seconds before the solvent was drained through the frit each time. To the resin was added piperidine:DMF (20:80 v/v, 3.75 mL). The mixture was periodically agitated for 5.0 minutes and then the solution was drained through the frit. To the reaction vessel was added piperidine:DMF (20:80 v/v, 3.75 mL). The mixture was periodically agitated for 5.0 minutes and then the solution was drained through the frit. The resin was washed successively six times as follows: for each wash, DMF (2.5 mL) was added to the vessel and the resulting mixture was periodically agitated for 30 seconds before the solution was drained through the frit. To the reaction vessel was added the amino acid (0.2 M in DMF, 2.5 mL, 10 equiv), then HATU (0.4 M in DMF, 1.25 mL, 10 equiv), and finally NMM (0.8 M in DMF, 1.25 mL, 20 equiv). The mixture was periodically agitated for 30-120 minutes, then the reaction solution was drained through the frit. The resin was washed successively six times as follows: for each wash, DMF (2.5 mL) was added and the resulting mixture was periodically agitated for 30 seconds before the solution was drained through the frit. The resulting resin was used directly in the next step.

### Single-Coupling Pre-Activation Procedure:

To the reaction vessel containing the resin from the previous step was added DMF (3.75 mL) three times, upon which the mixture was agitated for 30 seconds before the solvent was drained through the frit each time. To the resin was added piperidine:DMF (20:80 v/v, 3.75 mL). The mixture was periodically agitated for 5.0 minutes and then the solution was drained through the frit. To the reaction vessel was added piperidine:DMF (20:80 v/v, 3.75 mL). To the resin was added piperidine:DMF (20:80 v/v, 3.75 mL). The mixture was periodically agitated for 5.0 minutes and then the solution was drained through the frit. The mixture was periodically agitated for 5.0 minutes and then the solution was drained through the frit. The resin was washed successively six times as follows: for each wash, DMF (3.75 mL) was added to the vessel and the resulting mixture was periodically agitated for 30 seconds before the solution was drained through the frit. To the reaction vessel was added the premixed amino acid and HATU (0.1 M in DMF, 1.25 mL, 1:1 ratio 2.5 equiv), then NMM (0.8 M in DMF, 1.25 mL, 20 equiv). The mixture was periodically agitated for 2-3 hours, then the reaction solution was drained through the frit. The resin was washed successively four times as follows: for each wash, DMF (3.75 mL) was added and the resulting mixture was periodically agitated for 30 seconds before the solution was drained through the frit. The resulting resin was used directly in the next step.

### Double-Coupling Procedure:

To the reaction vessel containing resin from the previous step was added DMF (2.5 mL) three times, upon which the mixture was agitated for 30 seconds before the solvent was drained through the frit each time. To the reaction vessel was added piperidine:DMF (20:80 v/v, 3.75 mL). The mixture was periodically agitated for 5 minutes and then the solution was drained through the frit. To the reaction vessel was added piperidine:DMF (20:80 v/v, 3.75 mL). The mixture was periodically agitated for 5 minutes and then the solution was drained through the frit. The resin was washed successively six times as follows: for each wash, DMF (3.75 mL) was added and the resulting mixture was periodically agitated for 30 seconds before the solution was drained through the frit. To the reaction vessel was added the amino acid (0.2 M in DMF, 2.5 mL, 10 equiv), then HATU (0.4 M in DMF, 1.25 mL, 10 equiv), and finally NMM (0.8 M in DMF, 1.25 mL, 20 equiv). The mixture was periodically agitated for 1 hour, then the reaction solution was drained through the frit. The resin was washed twice with DMF (3.75 mL) and the resulting mixture was periodically agitated for 30 seconds before the solution was drained through the frit each time. To the reaction vessel was added the amino acid (0.2 M in DMF, 2.5 mL, 10 equiv), then HATU (0.4 M in DMF, 1.25 mL, 10 equiv), and finally NMM (0.8 M in DMF, 1.25 mL, 20 eq). The mixture was periodically agitated for 1-2 hours, then the reaction solution was drained through the frit. The resin was successively washed six times as follows: for each wash, DMF (3.75 mL) was added and the resulting mixture was periodically agitated for 30 seconds before the solution was drained through the frit. The resulting resin was used directly in the next step.

### Chloroacetic Anhydride Coupling:

To the reaction vessel containing resin from the previous step was added DMF (3.75 mL) three times, upon which the mixture was agitated for 30 seconds before the solvent was drained through the frit each time. To the reaction vessel containing the resin from the previous step was added piperidine:DMF (20:80 v/v, 3.75 mL). The mixture was periodically agitated for 5 minutes and then the solution was drained through the frit. To the reaction vessel was added piperidine:DMF (20:80 v/v, 3.75 mL). The mixture was periodically agitated for 5 minutes and then the solution was drained through the frit. The resin was washed successively six times as follows: for each wash, DMF (3.75 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NMM (0.8 M in DMF, 2.5 mL, 40 equiv). The mixture was periodically agitated for 15 minutes, then the reaction solution was drained through the frit. The resin was washed once as follows: DMF (6.25 mL) was added through the top of the vessel and the resulting mixture was periodically agitated for 30 seconds before the solution was drained through the frit. To the reaction vessel was added the chloroacetic anhydride solution (0.4 M in DMF, 3.75 mL, 30 equiv), then NMM (0.8 M in DMF, 2.5 mL, 40 equiv). The mixture was periodically agitated for 15 minutes, then the reaction solution was drained through the frit. The resin was washed successively six times as follows: for each wash, DMF (2.5 mL) was added through the top of the vessel and the resulting mixture was periodically agitated for 30 seconds before the solution was drained through the frit. The resin was washed successively four times as follows: for each wash, DCM (2.5 mL) was added through the top of the vessel and the resulting mixture was periodically agitated for 30 seconds before the solution was drained through the frit. The resulting resin was dried using a nitrogen flow for 10 mins before being used directly in the next step.

### Symphony X Methods:

All manipulations were performed under automation on a Symphony X peptide synthesizer (Protein Technologies). Unless noted, all procedures were performed in a 45-mL polypropylene reaction vessel fitted with a bottom frit. The reaction vessel connects to the Symphony X peptide synthesizer through both the bottom and the top of the vessel. DMF and DCM can be added through the top of the vessel, which washes down the sides of the vessel equally. The remaining reagents are added through the bottom of the reaction vessel and pass up through the frit to contact the resin. All solutions are removed through the bottom of the reaction vessel. “Periodic agitation” describes a brief pulse of N₂ gas through the bottom frit; the pulse lasts approximately 5 seconds and occurs every 30 seconds. A “single shot” mode of addition describes the addition of all the solution contained in the single shot falcon tube that is usually any volume less than 5 mL. Amino acid solutions were generally not used beyond two weeks from preparation. HATU solution was used within 14 days of preparation.

Sieber amide resin=9-Fmoc-aminoxanthen-3-yloxy polystyrene resin, where “3-yloxy” describes the position and type of connectivity to the polystyrene resin. The resin used is polystyrene with a Sieber linker (Fmoc-protected at nitrogen); 100-200 mesh, 1% DVB, 0.71 mmol/g loading.

Rink=(2,4-dimethoxyphenyl)(4-alkoxyphenyl)methanamine, where “4-alkoxy” describes the position and type of connectivity to the polystyrene resin. The resin used is Merrifield polymer (polystyrene) with a Rink linker (Fmoc-protected at nitrogen); 100-200 mesh, 1% DVB, 0.56 mmol/g loading.

2-Chlorotrityl chloride resin (2-Chlorotriphenylmethyl chloride resin), 50-150 mesh, 1% DVB, 1.54 mmol/g loading. Fmoc-glycine-2-chlorotrityl chloride resin, 200-400 mesh, 1% DVB, 0.63 mmol/g loading.

PL-FMP resin: (4-Formyl-3-methoxyphenoxymethyl)polystyrene.

Common amino acids used are listed below with side-chain protecting groups indicated inside parenthesis: Fmoc-Ala-OH; Fmoc-Arg(Pbf)-OH; Fmoc-Asn(Trt)-OH; Fmoc-Asp(tBu)-OH; Fmoc-Bip-OH; Fmoc-Cys(Trt)-OH; Fmoc-Dab(Boc)-OH; Fmoc-Dap(Boc)-OH; Fmoc-Gln(Trt)-OH; Fmoc-Gly-OH; Fmoc-His(Trt)-OH; Fmoc-Hyp(tBu)-OH; Fmoc-Ile-OH; Fmoc-Leu-OH; Fmoc-Lys(Boc)-OH; Fmoc-Nle-OH; Fmoc-Met-OH; Fmoc-[N-Me]Ala-OH; Fmoc-[N-Me]Nle-OH; Fmoc-Orn(Boc)-OH, Fmoc-Phe-OH; Fmoc-Pro-OH; Fmoc-Sar-OH; Fmoc-Ser(tBu)-OH; Fmoc-Thr(tBu)-OH; Fmoc-Trp(Boc)-OH; Fmoc-Tyr(tBu)-OH; Fmoc-Val-OH and their corresponding D-amino acids.

The procedures of “Symphony X Method” describe an experiment performed on a 0.050 mmol scale, where the scale is determined by the amount of Sieber or Rink or 2-chlorotrityl or PL-FMP bound to the resin. This scale corresponds to approximately 70 mg of the Sieber amide resin described above. All procedures can be scaled beyond or under 0.050 mmol scale by adjusting the described volumes by the multiple of the scale. Prior to amino acid coupling, all peptide synthesis sequences began with a resin-swelling procedure, described below as “Resin-swelling procedure”. Coupling of amino acids to a primary amine N-terminus used the “Single-coupling procedure” described below. Coupling of amino acids to a secondary amine N-terminus or to the N-terminus of Arg(Pbf)- and D-Arg(Pbf)- or D-Leu used the “Double-coupling procedure” or the “Single-Coupling 2-Hour Procedure” described below. Unless otherwise specified, the last step of automated synthesis is the acetyl group installation described as “Chloroacetyl Anhydride Installation”. All syntheses end with a final rinse and drying step described as “Standard final rinse and dry procedure”.

### Resin-Swelling Procedure:

To a 45-mL polypropylene solid-phase reaction vessel was added Sieber amide resin (70 mg, 0.050 mmol). The resin was washed (swelled) three times as follows: to the reaction vessel was added DMF (5.0 mL) through the top of the vessel “DMF top wash” upon which the mixture was periodically agitated for 3 minutes before the solvent was drained through the frit.

### Single-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Double-Coupling Procedure:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1 hour, then the reaction solution was drained through the frit. The resin was washed successively two times as follows: for each wash, DMF (5.0 mL) was added through the top of the vessel and the resulting mixture was periodically agitated for 30 seconds before the solution was drained through the frit. To the reaction vessel was added the amino acid (0.2 M in DMF, 2.0 mL, 8 equiv), then HATU (0.4 M in DMF, 1.0 mL, 8 equiv), and finally NMM (0.8 M in DMF, 1.0 mL, 16 equiv). The mixture was periodically agitated for 1-2 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure A:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, then the vessel was closed. The automatic program was resumed and HATU (0.4 M in DMF, 1.0 mL, 8 equiv) and NMM (0.8 M in DMF, 1.0 mL, 16 equiv) were added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Single-Coupling Manual Addition Procedure B:

To the reaction vessel containing the resin from the previous step was added piperidine:DMF (20:80 v/v, 4.0 mL). The mixture was periodically agitated for 5 minutes and then the solution was drained through the frit. To the reaction vessel was added piperidine:DMF (20:80 v/v, 4.0 mL). The mixture was periodically agitated for 5 minutes and then the solution was drained through the frit. The resin was washed successively six times as follows: for each wash, DMF (5.0 mL) was added through the top of the vessel and the resulting mixture was periodically agitated for 30 seconds before the solution was drained through the frit. The reaction was paused. The reaction vessel was opened and the unnatural amino acid (2-4 equiv) in DMF (1-1.5 mL) was added manually using a pipette from the top of the vessel while the bottom of the vessel remained attached to the instrument, followed by the manual addition of HATU (2-4 equiv, same equiv as the unnatural amino acid), then the vessel was closed. The automatic program was resumed and NMM (0.8 M in DMF, 1.0 mL, 16 equiv) was added sequentially. The mixture was periodically agitated for 2-3 hours, then the reaction solution was drained through the frit. The resin was washed successively five times as follows: for each wash, DMF (5.0 mL) was added through the top of the vessel and the resulting mixture was periodically agitated for 30 seconds before the solution was drained through the frit. The resulting resin was used directly in the next step.

### Chloroacetic Anhydride Coupling:

To the reaction vessel containing the resin from the previous step was added piperidine:DMF (20:80 v/v, 3.0 mL). The mixture was periodically agitated for 3.5 or 5 minutes and then the solution was drained through the frit. To the reaction vessel was added piperidine:DMF (20:80 v/v, 3.0 mL). The mixture was periodically agitated for 5 minutes and then the solution was drained through the frit. The resin was washed successively six times as follows: for each wash, DMF (3.0 mL) was added through the top of the vessel and the resulting mixture was periodically agitated for 30 seconds before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed twice as follows: for each wash, DMF (3.0 mL) was added through the top of the vessel and the resulting mixture was periodically agitated for 1.0 minute before the solution was drained through the frit. To the reaction vessel was added the chloroacetic anhydride solution (0.4 M in DMF, 2.5 mL, 20 equiv), then N-methylmorpholine (0.8 M in DMF, 2.0 mL, 32 equiv). The mixture was periodically agitated for 15 minutes, then the reaction solution was drained through the frit. The resin was washed successively five times as follows: for each wash, DMF (3.0 mL) was added through the top of the vessel and the resulting mixture was periodically agitated for 1.0 minute before the solution was drained through the frit. The resulting resin was used directly in the next step.

### Final Rinse and Dry Procedure:

The resin from the previous step was washed successively six times as follows: for each wash, DCM (5.0 mL) was added through the top of the vessel and the resulting mixture was periodically agitated for 30 seconds before the solution was drained through the frit. The resin was then dried using a nitrogen flow for 10 minutes. The resulting resin was used directly in the next step.

### Global Deprotection Method A:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 50-mL falcon tube was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. To the suspension was added 35-50 mL of cold diethyl ether. The mixture was vigorously mixed upon which a significant amount of a white solid precipitated. The mixture was centrifuged for 3-5 minutes, then the solution was decanted away from the solids and discarded. The solids were suspended in Et₂O (30-40 mL); then the mixture was centrifuged for 3-5 minutes; and the solution was decanted away from the solids and discarded. For a final time, the solids were suspended in Et₂O (30-40 mL); the mixture was centrifuged for 3-5 minutes; and the solution was decanted away from the solids and discarded to afford the crude peptide as a white to off-white solid together with the cleaved resin after drying under a flow of nitrogen and/or under house vacuum. The crude was used at the same day for the cyclization step.

### Global Deprotection Method B:

Unless noted, all manipulations were performed manually. The procedure of “Global Deprotection Method” describes an experiment performed on a 0.050 mmol scale, where the scale is determined by the amount of Sieber or Rink or Wang or chlorotrityl resin or PL-FMP resin. The procedure can be scaled beyond 0.05 mmol scale by adjusting the described volumes by the multiple of the scale. In a 30-ml Bio-Rad poly-prep chromatography column was added the resin and 2.0-5.0 mL of the cleavage cocktail (TFA:TIS:DTT, v/v/w=94:5:1). The volume of the cleavage cocktail used for each individual linear peptide can be variable. Generally, higher number of protecting groups present in the sidechain of the peptide requires larger volume of the cleavage cocktail. The mixture was shaken at room temperature for 1-2 hours, usually about 1.5 hour. The acidic solution was drained into 40 mL of cold diethyl ether and the resin was washed twice with 0.5 mL of TFA. The mixture was centrifuged for 3-5 minutes, then the solution was decanted away from the solids and discarded. The solids were suspended in Et₂O (35 mL); then the mixture was centrifuged for 3-5 minutes; and the solution was decanted away from the solids and discarded. For a final time, the solids were suspended in Et₂O (35 mL); the mixture was centrifuged for 3-5 minutes; and the solution was decanted away from the solids and discarded to afford the crude peptide as a white to off-white solid after drying under a flow of nitrogen and/or under house vacuum. The crude was used at the same day for the cyclization step.

### Cyclization Method A:

Unless noted, all manipulations were performed manually. The procedure of “Cyclization Method A” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids from the global deprotection were dissolved in DMF (30-45 mL) in the 50-mL centrifuge tube at room temperature, and to the solution was added DIEA (1.0-2.0 mL) and the pH value of the reaction mixture above was 8. The solution was then allowed to shake for several hours or overnight or over 2-3 days at room temperature. The reaction solution was concentrated to dryness on a speedvac or Genevac EZ-2 and the crude residue was then dissolved in DMF or DMF/DMSO (2 mL). After filtration, this solution was subjected to single compound reverse-phase HPLC purification to afford the desired cyclic peptide.

### Cyclization Method B:

Unless noted, all manipulations were performed manually. The procedure of “Cyclization Method B” describes an experiment performed on a 0.05 mmol scale, where the scale is determined by the amount of Sieber or Rink or chlorotrityl or Wang or PL-FMP resin that was used to generate the peptide. This scale is not based on a direct determination of the quantity of peptide used in the procedure. The procedure can be scaled beyond 0.05 mmol scale by adjusting the described volumes by the multiple of the scale. The crude peptide solids in the 50-mL centrifuge tube were dissolved in CH₃CN/0.1 M aqueous solution of ammonium bicarbonate (1:1, v/v, 30-45 mL). The solution was then allowed to shake for several hours at room temperature. The reaction solution was checked by pH paper and LCMS, and the pH can be adjusted to above 8 by adding 0.1 M aqueous ammonium bicarbonate (5-10 mL). After completion of the reaction based on the disappearance of the linear peptide on LCMS, the reaction was concentrated to dryness on a speedvac or Genevac EZ-2. The resulting residue was charged with CH₃CN:H₂O (2:3, v/v, 30 mL), and concentrated to dryness on a speedvac or Genevac EZ-2. This procedure was repeated (usually 2 times). The resulting crude solids were then dissolved in DMF or DMF/DMSO or CH₃CN/H₂O/formic acid. After filtration, the solution was subjected to single compound reverse-phase HPLC purification to afford the desired cyclic peptide.

N-Methylation on-resin Method A. To the resin (50 μmol) in a Bio-Rad tube was added CH₂Cl₂ (2 mL) and shaken for 5 min at RT 2-Nitrobenzene-1-sulfonyl chloride (44.3 mg, 200 μmol, 4 equiv) was added followed by the addition of 2,4,6-trimethylpyridine (0.040 mL, 300 μmol, 6 equiv). The reaction was shaken at RT for 2 h. The solvent was drained and the resin was rinsed with CH₂Cl₂ (5 mL×3), DMF (5 mL×3) and then THF (5 mL×3). The resin was added THE (1 mL). Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at RT for 2-16 h. The reaction was repeated. Triphenylphosphine (65.6 mg, 250 μmol, 5 equiv), methanol (0.020 mL, 500 μmol, 10 equiv) and Diethyl azodicarboxylate or DIAD (0.040 mL, 250 μmol, 5 equiv) were added. The mixture was shaken at RT for 1-16 h. The solvent was drained, and the resin was washed with THE (5 mL×3) and CHCl₃ (5 mL×3). The resin was air dried and used directly in the next step. The resin was shaken in DMF (2 mL). 2-Mercaptoethanol (39.1 mg, 500 μmol) was added followed by DBU (0.038 mL, 250 μmol, 5 equiv). The reaction was shaken for 1.5 h. The solvent was drained. The resin was washed with DMF (4×) air dried and used directly in the next step.

N-Methylation on-resin Method B (Turner, R. A. et al, Org. Lett., 15(19):5012-5015 (2013)). All manipulations were performed manually unless noted. The procedure of “N-methylation on-resin Method A” describes an experiment performed on a 0.100 mmol scale, where the scale is determined by the amount of Sieber or Rink linker bound to the resin that was used to generate the peptide. This scale is not based on a direct determination of the quantity of peptide used in the procedure. The procedure can be scaled beyond 0.10 mmol scale by adjusting the described volumes by the multiple of the scale. The resin was transferred into a 25 mL fritted syringe. To the resin was added piperidine:DMF (20:80 v/v, 5.0 mL). The mixture was shaken for 3 min. and then the solution was drained through the frit. The resin was washed 3 times with DMF (4.0 mL). To the reaction vessel was added piperidine:DMF (20:80 v/v, 4.0 mL). The mixture was shaken for 3 min. and then the solution was drained through the frit. The resin was washed successively three times with DMF (4.0 mL) and three times with DCM (4.0 mL). The resin was suspended in DMF (2.0 mL) and ethyl trifluoroacetate (0.119 ml, 1.00 mmol), 1,8-diazabicyclo[5.4.0]undec-7-ene (0.181 ml, 1.20 mmol). The mixture was placed on a shaker for 60 min. The solution was drained through the frit. The resin was washed successively three times with DMF (4.0 mL) and three times with DCM (4.0 mL). The resin was washed three times with dry THE (2.0 mL) to remove any residual water. In an oven dried 4.0 mL vial was added THE (1.0 mL) and triphenylphosphine (131 mg, 0.500 mmol) over dry 4 Å molecular sieves (20 mg). The solution was transferred to the resin and diisopropyl azodicarboxylate (0.097 mL, 0.5 mmol) was added slowly. The resin was stirred for 15 min. The solution was drained through the frit and the resin was washed three times with dry THF (2.0 mL) to remove any residual water. In an oven dried 4.0 mL vial was added THE (1.0 mL), and triphenylphosphine (131 mg, 0.50 mmol) over dry 4 A molecular sieves (20 mg). The solution was transferred to the resin and diisopropyl azodicarboxylate (0.097 mL, 0.5 mmol) was added slowly. The resin was stirred for 15 min. The solution was drained through the frit. The resin was washed successively three times with DMF (4.0 mL) and three times with DCM (4.0 mL). The resin was suspended in Ethanol (1.0 mL) and THE (1.0 mL), and sodium borohydride (37.8 mg, 1.000 mmol) was added. The mixture was stirred for 30 min. and drained. The resin was washed successively three times with DMF (4.0 mL) and three times with DCM (4.0 mL).

### N-Alkylation On-Resin Procedure Method A:

A solution of the alcohol corresponding to the alkylating group (0.046 g, 1.000 mmol), triphenylphosphine (0.131 g, 0.500 mmol), and DIAD (0.097 mL, 0.500 mmol) in 3 mL of THE was added to nosylated resin (0.186 g, 0.100 mmol), and the reaction mixture was stirred for 16 hours at room temperature. The resin was washed three times with THE (5 mL), and the above procedure was repeated 1-3 times. Reaction progress was monitored by TFA micro-cleavage of small resin samples treated with a solution of 50 μL of TIS in 1 mL of TFA for 1.5 hours.

### N-Alkylation On-Resin Procedure Method B:

The nosylated resin (0.100 mmol) was washed three times with N-methylpyrrolidone (NMP) (3 mL). A solution of NMP (3 mL), Alkyl Bromide (20 eq, 2.000 mmol) and DBU (20 eq, 0.301 mL, 2.000 mmol) was added to the resin, and the reaction mixture was stirred for 16 hours at room temperature. The resin was washed with NMP (3 mL) and the above procedure was repeated once more. Reaction progress was monitored by TFA micro-cleavage of small resin samples treated with a solution of 50 μL of TIS in 1 mL of TFA for 1.5 hours.

### N-Nosylate Formation Procedure:

A solution of collidine (10 eq.) in DCM (2 mL) was added to the resin, followed by a solution of Nos-Cl (8 eq.) in DCM (1 mL). The reaction mixture was stirred for 16 hours at room temperature. The resin was washed three times with DCM (4 mL) and three times with DMF (4 mL). The alternating DCM and DMF washes were repeated three times, followed by one final set of four DCM washes (4 mL).

### N-Nosylate Removal Procedure:

The resin (0.100 mmol) was swelled using three washes with DMF (3 mL) and three washes with NMP (3 mL). A solution of NMP (3 mL), DBU (0.075 mL, 0.500 mmol) and 2-mercaptoethanol (0.071 mL, 1.000 mmol) was added to the resin and the reaction mixture was stirred for 5 minutes at room temperature. After filtering and washing with NMP (3 mL), the resin was re-treated with a solution of NMP (3 mL), DBU (0.075 mL, 0.500 mmol) and 2-mercaptoethanol (0.071 mL, 1.000 mmol) for 5 minutes at room temperature. The resin was washed three times with NMP (3 mL), four times with DMF (4 mL) and four times with DCM (4 mL), and was placed back into a Symphony reaction vessel for completion of sequence assembly on the Symphony peptide synthesizer.

### General Procedure for Preloaded Amines on the PL-FMP Resin:

PL-FMP resin (Novabiochem, 1.00 mmol/g substitution) was swollen with DMF (20 mL/mmol) at room temperature. The solvent was drained and 10 ml of DMF was added, followed by the addition of the amine (2.5 mmol) and acetic acid (0.3 mL) into the reaction vessel. After 10-min agitation, sodium triacetoxyhydroborate (2.5 mmol) was added. The reaction was allowed to agitate overnight. The resin was washed with DMF (1×), THF/H₂O/AcOH (6:3:1) (2×), DMF (2×), DCM (3×), and dried. The resulting PL-FMP resin preloaded with the amine can be checked by the following method: Took 100 mg of above resin and reacted with benzoyl chloride (5 equiv), and DIEA (10 equiv) in DCM (2 mL) at room temperature for 0.5 h. The resin was washed with DMF (2×), MeOH (lx), and DCM (3×). The sample was then cleaved with 40% TFA/DCM (1 h). The product was collected and analyzed by HPLC and MS. Collected sample was dried and got weight to calculate resin loading.

### Click Reaction On-Resin Procedure Method A:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swelled with DCM (2×5 mL×5 mins) and then DMF (2×5 mL×5 mins). In a 200-ml bottle was charged with 30 fold of the following: vitamin C (0.026 g, 0.150 mmol), bis(2,2,6,6-tetramethyl-3,5-heptanedionato)copper(II) (10.75 mg, 0.025 mmol), DMF (1.5 mL), 2,6-lutidine (0.058 mL, 0.50 mmol) and THE (1.5 ml), followed by DIPEA (0.087 ml, 0.50 mmol) and the azide, tert-butyl (S)-1-azido-40-(tert-butoxycarbonyl)-37,42-dioxo-3,6,9,12,15,18,21,24,27,30,33-undecaoxa-36,41-diazanonapentacontan-59-oate (0.028 g, 0.025 mmol). The mixture was stirred until everything was in solution. The DMF in the above Bio-Rad tube was drained, and the above click solution (3 mL each) was added to each Bio-Rad tube. The tubes were shaken overnight on an orbital shaker. Solutions were drained through the frit. The resins were washed with DMF (3×2 mL) and DCM (3×2 mL).

### Click Reaction On-Resin Procedure Method B:

This procedure describes an experiment performed on a 0.050 mmol scale. It can be scaled beyond or under 0.050 mmol scale by adjusting the described volumes by the multiple of the scale. The alkyne containing resin (50 μmol each) was transferred into Bio-Rad tubes and swelled with DCM (2×5 mL×5 mins) and then DMF (2 5 mL×5 mins). In a separate bottle, nitrogen was bubbled into 4.0 mL of DMSO for 15 mins. To the DMSO was added copper iodide (9.52 mg, 0.050 mmol, 1.0 eq) (sonicated), lutidine (58 μL, 0.500 mmol, 10.0 eq) and DIEA (87 uL, 0.050 mmol, 10.0 eq). The solution was purged with nitrogen again. DCM was drained through the frit. In a separate vial, ascorbic acid (8.8 mg, 0.050 mmol, 1.0 eq) was dissolved into water (600 uL). Nitrogen was bubbled through the solution for 10 mins. Coupling partners were distributed in the tubes (0.050 mmol to 0.10 mmol, 1.0 to 2.0 eq) followed by the DMSO copper and base solution and finally ascorbic acid aqueous solution. The solutions were topped with a blanket of nitrogen and capped. The tube was put onto the rotatory mixer for 16 hours. Solutions were drained through the frit. The resins were washed with DMF (3×2 mL) and DCM (3×2 mL).

### Suzuki Reaction On-Resin Procedure:

In a Bio Rad tube is placed 50 umoles of dried Rink resin of a N-terminus Fmoc-protected linear polypeptide containing a 4-bromo-phenylalanine side chain. The resin was swelled with DMF (2×5 mL). To this was added a DMF solution (2 mL) of p-tolylboronic acid (0.017 g, 0.125 mmol), potassium phosphate (0.2 mL, 0.400 mmol) followed by the catalyst [1′-bis(di-tert-butylphosphino)ferrocene]dichloropalladiun(II) [PdCl₂(dthpf)] (3.26 mg, 5.00 μmol). The tube was shaken at RT overnight. The solution was drained and the resin was washed with DMF (5×3 mL) followed by alternating DCM (2×3 mL), then DMF (2×3 mL), and then DCM (5×3 mL). A small sample of resin was micro-cleaved using 235 μL of TIS in 1 ml TFA at RT for 1 h. The rest of the resin was used in the next step of peptide coupling or chloroacetic acid capping of the N-terminus.

### Preparation of (S)-2-((((9H-fluoren-9-yl)methoxy)carbonyl)-3-(1-(2-(tert-butoxy)-2-oxoethyl)-1H-indol-3-yl)propanoic acid

Step 1: To a 0° C. solution of (S)-benzyl 2-(((benzyloxy)carbonyl)amino)-3-(1H-indol-3-yl) propanoate (25.0 g, 58.3 mmol) and cesium carbonate (20.9 g, 64.2 mmol) in DMF (200 mL) was added tert-butyl 2-bromoacetate (9.36 mL, 64.2 mmol). The solution was allowed to slowly warm up to RT with stirring for 18 h. The reaction mixture was poured into ice water:aq. 1N HCl (1:1) and then extracted with EtOAc. The organic layer was washed with brine, collected, dried over MgSO₄, filtered, and then concentrated in vacuo. The resulting solid was subjected to flash chromatography (330 g column, 0-50% EtOAc:Hex over 20 column volumes) to afford (S)-benzyl 2-(((benzyloxy)carbonyl)amino)-3-(1-(2-(tert-butoxy)-2-oxoethyl)-1H-indol-3-yl)propanoate as a white solid (29.6 g, 93%).

Step 2: H₂ was slowly bubbled through a mixture of (S)-benzyl 2-(((benzyloxy)carbonyl)amino)-3-(1-(2-(tert-butoxy)-2-oxoethyl)-1H-indol-3-yl)propanoate (29.6 g, 54.5 mmol) and Pd—C(1.45 g, 1.36 mmol) in MeOH (200 mL) at RT for 10 min. The mixture was then stirred under positive pressure of H₂ while conversion was monitored by LCMS. After 48 h the reaction mixture was filtered through diatomaceous earth and evaporated to afford crude (S)-2-amino-3-(1-(2-(tert-butoxy)-2-oxoethyl)-1H-indol-3-yl)propanoic acid (17.0 g) which was carried into step three without additional purification.

Step 3: To a solution of (S)-2-amino-3-(1-(2-(tert-butoxy)-2-oxoethyl)-1H-indol-3-yl)propanoic acid (5.17 g, 16.2 mmol) and sodium bicarbonate (6.8 g, 81 mmol) in acetone:water (50.0 mL:100 mL) was added (9H-fluoren-9-yl)methyl (2,5-dioxopyrrolidin-1-yl) carbonate (5.48 g, 16.2 mmol). The mixture stirred overnight upon which LCMS analysis indicated complete conversion. The vigorously stirred mixture was acidified via slow addition of aq 1N HCl. Once acidified, the mixture was diluted with DCM (150 mL), and the isolated organic phase was then washed with water, followed by brine. The organic layer was collected, dried over sodium sulfate, and concentrated under vacuum to afford the crude product. The crude material was purified via silica gel chromatography (330 g column, 20-80% EtOAc:Hex over 20 column 25 volumes) to afford (S)-2-((((9H-fluoren-9-yl)methoxy)carbonyl)amino)-3-(1-(2-(tertbutoxy)-2-oxoethyl)-1H-indol-3-yl)propanoic acid as a white foam (7.26 g, 83%). ¹H NMR (500 MHz, methanol-d₄) δ 7.80 (d, J=7.6 Hz, 2H), 7.67-7.60 (m, 2H), 7.39 (t, J=7.5 Hz, 2H), 7.32-7.22 (m, 3H), 7.18 (td, J=7.6, 0.9 Hz, 1H), 7.08 (td, J=7.5, 0.9 Hz, 1H), 7.04 (s, 1H), 4.54 (dd, J=8.4, 4.9 Hz, 1H), 4.36-4.23 (m, 2H), 4.23-4.14 (m, 1H), 30 3.43-3.35 (m, 2H), 3.25-3.09 (m, 1H), 1.55-1.38 (m, 9H). ESI-MS(+) m/z=541.3 (M+H).

### Preparation of (S)-2-((((9H-fluoren-9-yl)methoxy)carbonyl)amino)-3-(4-(2-(tert-butoxy)-2-oxoethoxy)phenyl)propanoic acid

Step 1. To a cooled stirred solution of (S)-benzyl 2-(((benzyloxy)carbonyl)amino)-3-(4-hydroxyphenyl)propanoate (70 g, 173 mmol) and K₂CO₃ (35.8 g, 259 mmol) in DMF (350 mL) was added tert-butyl-2-bromoacetate (30.6 mL, 207 mmol) dropwise and the resulting mixture was stirred at RT overnight. The reaction mixture was diluted with 10% brine solution (1000 mL) and extracted with ethyl acetate (2×250 mL). The combined organic layer was washed with water (500 mL), saturated brine solution (500 mL), dried over anhydrous sodium sulfate, filtered, and concentrated to afford a colorless gum. The crude compound was purified by flash column chromatography using 20% ethyl acetate in petroleum ether as an eluent to afford a white solid (78 g, 85%).

Step 2. The (S)-benzyl 2-(((benzyloxy)carbonyl)amino)-3-(4-(2-(tert-butoxy)-2-oxoethoxy)phenyl)propanoate (73 g, 140 mmol) was dissolved in MeOH (3000 mL) and purged with nitrogen for 5 min. To the above purged mixture was added Pd/C (18 g, 16.91 mmol) and stirred under hydrogen pressure of 3 kg for 15 hours. The reaction mixture was filtered through a bed of diatomaceous earth (Celite©) and washed with methanol (1000 mL). The filtrate was concentrated under vacuum to afford a white solid (36 g, 87%).

Step 3. To a stirred solution of (S)-2-amino-3-(4-(2-(tert-butoxy)-2-oxoethoxy)phenyl)propanoic acid (38 g, 129 mmol) and sodium bicarbonate (43.2 g, 515 mmol) in water (440 mL) was added Fmoc-OSu (43.4 g, 129 mmol) dissolved in dioxane (440 mL) dropwise and the resulting mixture was stirred at RT overnight. The reaction mixture was diluted with 1.5 N HCl (200 mL) and water (500 mL) and extracted with ethyl acetate (2×250 mL). The combined organic layer was washed with water (250 mL), saturated brine solution (250 mL), and dried over Na₂SO₄, filtered, and concentrated to afford a pale yellow gum. The crude compound was purified by column chromatography using 6% MeOH in chloroform as an eluent to afford a pale green gum. The gum was further triturated with petroleum ether to afford an off-white solid (45 g, 67%). ¹H NMR (400 MHz, DMSO-d₆) δ 12.86-12.58 (m, 1H), 7.88 (d, J=7.5 Hz, 2H), 7.73-7.61 (m, 3H), 7.58-7.47 (m, 1H), 7.44-7.27 (m, 4H), 7.18 (d, J=8.5 Hz, 2H), 6.79 (d, J=8.5 Hz, 2H), 4.57 (s, 2H), 4.25-4.10 (m, 4H), 3.34 (br s, 3H), 3.02 (dd, J=13.8, 4.3 Hz, 1H), 2.81 (dd, J=14.1, 10.5 Hz, 1H), 1.41 (s, 9H).

### Preparation of (S)-2-((((9H-fluoren-9-yl)methoxy)carbonyl)amino)-3-(4-(tert-butoxycarbonyl)phenyl)propanoic acid

Step 1. (S)-Benzyl 2-(((benzyloxy)carbonyl)amino)-3-(4-hydroxyphenyl)propanoate (10 g, 24.66 mmol) was taken in DCM (100 mL) in a 250 mL multi-neck round bottom flask under magnetic stirring with N₂ outlet. The reaction mixture was cooled to −40° C., pyridine (5.49 mL, 67.8 mmol) was added slowly and then stirred at the same temperature for 20 minutes, followed by addition of triflic anhydride (11.46 mL, 67.8 mmol) slowly at −40° C. and allowed to stir at −40° C. for 2 hours. The reaction mixture was quenched with water at −10° C., and then citric acid solution (50 mL) was added. The organic layer was extracted with DCM, and the separated organic layer was dried over anhydrous Na₂SO₄, filtered, and then evaporated to give (S)-benzyl 2-(((benzyloxy)carbonyl)amino)-3-(4-(((trifluoromethyl)sulfonyl)oxy)phenyl)propanoate (11.93 g, 22.20 mmol, 90% yield) as a pale yellow solid.

Step 2. A solution of DMF (1500 mL) was purged with nitrogen for 10 min. To this was added sodium formate (114 g, 1676 mmol) and acetic anhydride (106 mL, 1123 mmol). Purging continued and the mixture was cooled to 0° C. DIPEA (194 mL, 1111 mmol) was added and the reaction mixture was allowed to stir for 1 h at RT under nitrogen atmosphere.

To a 10-liter autoclave was added DMF (3200 mL) and the system was purged with nitrogen. Under the nitrogen purging conditions, (S)-benzyl 2-(((benzyloxy)carbonyl)amino)-3-(4-(((trifluoromethyl)sulfonyl)oxy)phenyl)propanoate (300 g, 558 mmol), lithium chloride (71 g, 1675 mmol), 1,3-bis(diphenylphosphino)propane (24.17 g, 58.6 mmol) were added followed by the addition of palladium(II) acetate (12.9 g, 57.5 mmol). To this reaction mixture was added the above prepared solution and heated to 80° C. for 16 h.

The reaction mass was diluted with ethyl acetate and water. The phases were separated and the ethyl acetate layer was washed with water and brine solution, dried over anhydrous sodium sulphate, filtered, and concentrated. The crude material was added to a torrent column and was eluted with petroleum ether and ethyl acetate. The fractions at 30%-65% ethyl acetate in petroleum ether were concentrated to afford a cream solid (300 g), which was dissolved in ethyl acetate (700 mL) and petroleum ether was added slowly. At about 20% ethyl acetate in petroleum ether a white solid precipitated out, which was filtered and washed with 20% ethyl acetate in petroleum ether to obtain a white solid (180 g, yield 74%).

Step 3. To a 2000-ml multi-neck round-bottomed flask was charged (S)-4-(3-(benzyloxy)-2-(((benzyloxy)carbonyl)amino)-3-oxopropyl)benzoic acid (130 g, 300 mmol), dichloromethane (260 mL) and cyclohexane (130 mL). To the slurry reaction mixture was added BF₃·OEt₂ (3.80 mL, 30.0 mmol) at room temperature, followed by the addition of tert-butyl 2,2,2-trichloroacetimidate (262 g, 1200 mmol) slowly at room temperature over 30 min. Upon addition, the slurry slowly started dissolving and at the end of the addition it was completely dissolved. The reaction mixture was allowed to stir at room temperature for 16 h. The reaction mixture was diluted with DCM and the remaining solids were removed by filtration. The filtrate was concentrated and purified by flash chromatography. The crude material was purified by Torrent using a 1.5 Kg silicycle column. The product spot was eluted with a 15% ethyl acetate/petroleum ether mixture. The collected fractions were concentrated to obtain a colorless liquid (120 g, yield 82%).

Step 4. (S)-tert-Butyl 4-(3-(benzyloxy)-2-(((benzyloxy)carbonyl)amino)-3-oxopropyl)benzoate (200 g, 409 mmol) was dissolved in MeOH (4000 mL) and N₂ was purged for 10 min. Pd/C (27.4 g, 25.7 mmol) was added. The reaction was shaken under H₂ for 16 h at room temperature. The reaction mass was filtered through a celite bed and the bed was washed with methanol. The obtained filtrate was concentrated to obtain a pale yellow solid. The obtained solid was stirred with 5% methanol:diethyl ether mixture for 15 min before being filtered, and dried under vacuum to obtain a pale yellow solid. It was made slurry with 5% methanol in diethyl ether and stirred for 15 min, filtered, and dried to give (S)-2-amino-3-(4-(tert-butoxycarbonyl)phenyl)propanoic acid as a white solid (105 g, yield 97%). Analysis condition E: Retention time=0.971 min; ESI-MS(+) m/z [M+H]⁺: 266.2.

Step 5. (S)-2-Amino-3-(4-(tert-butoxycarbonyl)phenyl)propanoic acid (122 g, 460 mmol) was dissolved in acetone (1000 mL) and then water (260 mL) and sodium bicarbonate (116 g, 1380 mmol) were added. The reaction was cooled to 0° C. and Fmoc-OSu (155 g, 460 mmol) was added portionwise into the reaction mixture. After completion of addition it was stirred at room temperature for 16 h. The reaction mixture was diluted with dichloromethane (2 L) and then water was added (1.5 L). The organic layer was washed with saturated citric acid solution and extracted, and the aqueous layer was again extracted with DCM. The combined organic layer was washed with 10% citric acid solution, brine solution, and dried over Na₂SO₄, and evaporated to dryness. The obtained white solid was made slurry with diethyl ether, filtered, and dried to provide the desired product as a white solid (80 g, yield 35%). ¹H NMR (400 MHz, DMSO-d₆) δ 7.87 (d, J=7.5 Hz, 2H), 7.83-7.73 (m, 3H), 7.60 (t, J=8.5 Hz, 2H), 7.51-7.24 (m, 7H), 4.26-4.11 (m, 4H), 3.45-3.27 (m, 4H), 3.17 (br dd, J=13.8, 4.3 Hz, 1H), 2.94 (dd, J=13.5, 11.0 Hz, 1H), 2.52-2.48 (m, 4H), 1.51 (s, 9H).

### Preparation of tert-butyl (R)-2-((((9H-fluoren-9-yl)methoxy)carbonyl)amino)-3-iodopropanoate

Step 1. To a solution of (R)-2-amino-3-chloropropanoic acid hydrochloride (125 g, 781 mmol) in a 1:1 mixture of acetone (1 L) and water (1 L) was added Na₂CO₃ (182 g, 1719 mmol) followed by Fmoc-OSu (250 g, 742 mmol). The reaction was stirred at RT overnight. It was extracted with ethyl acetate (2×500 mL) and the aq. layer was acidified with 5N HCl. The HCl solution was extracted with ethyl acetate (1500 mL, then 2×500 mL). The combined organic layers were dried over anhydrous MgSO₄, filtered, and concentrated to give the crude product (R)-2-((((9H-fluoren-9-yl)methoxy)carbonyl)amino)-3-chloropropanoic acid. The product (220 g) was taken to the next step as such.

Step 2. A solution of (R)-2-((((9H-fluoren-9-yl)methoxy)carbonyl)amino)-3-chloropropanoic acid (220 g, 636 mmol) in DCM (2 L) was cooled to −20° C. 2-Methylpropene (200 mL, 636 mmol) was bubbled into the solution for 15 mins, then H₂SO₄ (57.7 mL, 1082 mmol) was added and the mixture was stirred at RT overnight. To the reaction mixture was added water (500 mL). The layers were separated and the aqueous layer was extracted with DCM (2×500 mL). The combined organic layers were dried over anhydrous MgSO₄, filtered, and evaporated. The crude was purified by flash chromatography using petroleum ether and ethyl acetate elution solvents. The desired fractions were combined and concentrated to give the product (R)-tert-butyl 2-((((9H-fluoren-9-yl)methoxy)carbonyl)amino)-3-chloropropanoate (83 g, 182 mmol, 29% yield).

Step 3. To a solution of (R)-tert-butyl 2-((((9H-fluoren-9-yl)methoxy)carbonyl)amino)-3-chloropropanoate (80 g, 199 mmol) in acetone (1000 mL) was added sodium iodide (119 g, 796 mmol) and the reaction was heated to reflux for 40 hours. Acetone was removed by rotavap and the crude product was diluted with water (1000 mL) and DCM (1000 mL). The layers were separated and the organic layer was washed with aqueous saturated sodium sulphite solution (1000 mL) and brine (1000 mL). The organic layer was dried over anhydrous Na₂SO₄, filtered, and concentrated. The crude was purified by flash chromatography using 7 to 9% of ethyl acetate in petroleum ether. The desired product fractions were combined and concentrated to afford the product (R)-tert-butyl 2-((((9H-fluoren-9-yl)methoxy)carbonyl)amino)-3-iodopropanoate (83 g, 156 mmol, 79%). ¹H NMR (400 MHz, CDCl₃) δ 7.77 (d, J=7.5 Hz, 2H), 7.62 (d, J=7.5 Hz, 2H), 7.45-7.30 (m, 4H), 5.67 (br d, J=7.0 Hz, 1H), 4.54-4.32 (m, 3H), 4.30-4.21 (m, 1H), 3.71-3.50 (m, 2H), 1.56-1.48 (m, 9H).

### Preparation of (S)-2-((((9H-fluoren-9-yl)methoxy)carbonyl)amino)-3-(2-methyl-1H-indol-3-yl)propanoic acid

Step 1. In a 100-ml three-neck, flame-dried, nitrogen-purged round-bottomed flask, zinc (2.319 g, 35.5 mmol) was added under argon atmosphere and the flask was heated to 150° C. using a heat gun and was purged with argon. To the reaction flask, DMF (50 mL) was added followed by the addition of 1,2-dibromoethane (0.017 mL, 0.20 mmol) and TMS-Cl (0.026 mL, 0.20 mmol) under argon atmosphere and then stirred for 10 min. To the reaction mixture (R)-tert-butyl 2-((((9H-fluoren-9-yl)methoxy)carbonyl)amino)-3-iodopropanoate (5 g, 10.14 mmol) was added and the reaction was stirred for 1 h. The reaction progress was monitored via TLC and LCMS, till the starting iodide was completely converted into the Zn-complex. The solution of organozinc reagent was allowed to cool to room temperature and then tris(dibenzylideneacetone)dipalladium(0) (Pd₂(dba)₃) (0.23 g, 0.25 mmol), dicyclohexyl(2′,6′-dimethoxy-[1,1′-biphenyl]-2-yl)phosphine (SPhos) (0.21 g, 0.51 mmol), and tert-butyl 3-bromo-2-methyl-1H-indole-1-carboxylate (3.77 g, 12.16 mmol) were added. The reaction mixture was allowed to stir at RT under a positive pressure of nitrogen for 1 h and then heated to 50° C. for 6 hrs. The reaction progress was monitored via LCMS. The mixture was diluted with EtOAc (700 mL) and filtered through Celite. The organic phase was washed with sat. NH₄Cl (250 mL), water (2×200 mL), and sat. NaCl (aq) (250 mL), dried over anhydrous Na₂SO₄(s), concentrated, and dried under vacuum to afford the crude compound (19 g). It was purified through ISCO flash chromatography a using 330 g RediSep column and the product was eluted with 7 to 9% of ethyl acetate in petroleum ether. The above reaction and purification were repeated. The pure fractions were concentrated to give tert-butyl (S)-3-(2-((((9H-fluoren-9-yl)methoxy)carbonyl)amino)-3-(tert-butoxy)-3-oxopropyl)-2-methyl-1H-indole-1-carboxylate as a brownish solid (10.2 g. 95% pure, ca. 80% yield). Analysis condition G: Retention time=4.23 min; ESI-MS(+) m/z [M+2H][M-Boc-tBu+H]⁺: 441.2.

Step 2. In a 25-ml multi neck, round-bottomed flask, DCM (65 mL) was added followed by (S)-tert-butyl 3-(2-((((9H-fluoren-9-yl)methoxy)carbonyl)amino)-3-(tert-butoxy)-3-oxopropyl)-2-methyl-1H-indole-1-carboxylate (6.5 g, 10.89 mmol) under nitrogen atmosphere at RT. The reaction mixture was cooled to 0° C., triethylsilane (4.18 mL, 26.1 mmol) was added followed by the addition of TFA (5.87 mL, 76 mmol) dropwise at 0° C. The temperature of the reaction mixture was slowly brought to RT and stirred at RT for 4 h. The reaction progress was monitored by TLC. To the reaction mixture, TFA (5.87 mL, 76 mmol) was added. The reaction mixture was stirred at RT overnight, and concentrated under reduced pressure. The crude material was triturated with hexanes and stored in a cold room to give a brown colored solid (crude weight: 6.5 g). It was purified via reverse phase flash chromatography, and the pure fractions were concentrated to obtain the desired final product as an off-white powder (2.3 g, 46%). ¹H NMR (DMSO-d₆): δ ppm: 10.65 (s, 1H), 7.84 (d, J=9.12 Hz, 2H), 7.65 (d, J=9.12 Hz, 2H), 7.42-7.49 (m, 1H), 7.30-7.38 (m, 2H), 7.26-7.29 (m, 2H), 7.17-7.19 (m, 2H), 6.91-6.95 (m, 1H), 6.85-6.88 (t, J=7.85 Hz, 1H), 4-16-4.18 (m, 2H), 4.01-4.06 (m, 1H), 3.09-3.14 (m, 1H), 2.96-2.99 (m, 1H), 2.50 (s, 3H). Analysis condition F: Retention time=1.37 min; ESI-MS(+) m/z [M+2H][M+H]⁺: 441.2.

### Preparation of (S)-2-((((9H-fluoren-9-yl)methoxy)carbonyl)amino)-3-(7-methyl-1H-indol-3-yl)propanoic acid

Step 1. In a 50-mi round-bottomed flask, dry zinc (0.928 g, 14.19 mmol) was charged and flushed with argon three times and then the flask was heated to 150° C. for 5 min and then allowed to cool to room temperature and flushed with argon 3 times. DMF (20 mL) was added followed by the addition of 1,2-dibromoethane (6.99 μl, 0.081 mmol) and TMS-Cl (0.013 mL, 0.10 mmol). Successful zinc insertion was accompanied by a noticeable exotherm. After 5 min (R)-tert-butyl 2-((((9H-fluoren-9-yl)methoxy)carbonyl)amino)-3-iodopropanoate (2.0 g, 4.05 mmol) was added and the reaction was stirred for 30 min. In a 50-ml round-bottomed flask charged with Argon was added the above alkyl zinc reagent, tert-butyl 3-bromo-7-methyl-1H-indole-1-carboxylate (1.26 g, 4.05 mmol) followed by 2-dicyclohexylphosphino-2′,6′-dimethoxybiphenyl (SPhos) (0.083 g, 0.20 mmol) and Pd₂(dba)₃ (0.093 g, 0.101 mmol). After the addition the reaction mixture was heated to 50° C. overnight. Another equivalent of Sphos and Pd₂(dba)₃ were added and heating continued for another 16 h. The reaction mixture was diluted with EtOAc (100 mL) and filtered through Celite. The organic phase was washed with sat. aq. NH₄Cl (100 mL), water (50 mL), and sat NaCl (100 mL), dried over anhydrous Na₂SO₄(s), concentrated, and dried under vacuum. After purification by flash chromatography the desired tert-butyl (S)-3-(2-((((9H-fluoren-9-yl)methoxy)carbonyl)amino)-3-(tert-butoxy)-3-oxopropyl)-2-methyl-1H-indole-1-carboxylate was obtained in 58% yield.

Step 2. Final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(7-methyl-1H-indol-3-yl)propanoic acid as an off white solid in 64% yield after purification by reverse phase flash chromatography. Analysis condition E: Retention time=2.16 min; ESI-MS(+) m/z [M+H]⁺: 441.1. ¹H NMR (300 MHz, DMSO-d₆) Shift 12.70 (br s, 1H), 10.81 (br s, 1H), 7.88 (d, J=7.6 Hz, 2H), 7.76-7.56 (m, 2H), 7.49-7.21 (m, 5H), 7.17 (d, J=2.3 Hz, 1H), 6.94-6.84 (m, 2H), 4.29-4.13 (m, 3H), 4.07 (br s, 1H), 3.19 (br dd, J=14.7, 4.5 Hz, 1H), 3.01 (br dd, J=14.5, 9.6 Hz, 1H), 2.47-2.40 (m, 3H), 0.02-−0.06 (m, 1H).

### Preparation of(S)-2-((((9H-fluoren-9-yl)methoxy)carbonyl)amino)-3-(quinolin-6-yl)propanoic acid

Step 1. In a 25-ml round bottom flask, dry zinc (2.32 g, 35.5 mmol) was charged and argon was flushed three times. The flask was heated to 150° C. for 5 min and then allowed to cool to room temp and flushed with argon 3 times. DMF (50 mL) was added followed by the addition of 1,2-dibromoethane (0.017 mL, 0.20 mmol) and TMS-Cl (0.032 mL, 0.25 mmol). Successful zinc insertion was accompanied by a noticeable exotherm. After 5 min (R)-tert-butyl 2-((((9H-fluoren-9-yl)methoxy)carbonyl)amino)-3-iodopropanoate (5.0 g, 10.14 mmol) was added and the reaction was stirred for 30 min.

In a 250-ml round bottom flask purged with Argon was added DMF (50 mL), 6-bromoquinoline (2.53 g, 12.16 mmol), previously prepared solution of alkyl zinc reagent, (R)-tert-butyl 2-((((9H-fluoren-9-yl)methoxy)carbonyl)amino)-3-iodopropanoate (5.0 g, 10.14 mmol) followed by 2-dicyclohexylphosphino-2′,6′-diisopropoxy-1,1′-biphenyl (RuPhos) (0.24 g, 0.51 mmol) and Pd₂(dba)₃ (0.23 g, 0.25 mmol). The reaction mixture was allowed to stir at RT for 5 h and then heated to 50° C. for 16 h. It was cooled to RT and filtered over celite and rinsed with ethyl acetate. The solution was concentrated on a rotovap. Purification by flash chromatography gave the desired compound as a thick brown liquid in quantitative yield. Analysis condition E: Retention time=3.47 min; ESI-MS(+) m/z [M+H]⁺: 495.2.

Step 2. The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(quinolin-6-yl)propanoic acid as a beige solid in 40% yield after solid-liquid extraction with diethyl ether and water. ¹H NMR (300 MHz, DMSO-d₆) δ 8.94 (br d, J=4.5 Hz, 1H), 8.49 (d, J=8.7 Hz, 1H), 8.01-7.92 (m, 2H), 7.85-7.79 (m, 3H), 7.65 (dd, J=8.3, 4.5 Hz, 1H), 7.55 (dd, J=7.2, 4.2 Hz, 2H), 7.36 (t, J=7.4 Hz, 2H), 7.26-7.14 (m, 2H), 4.32 (dd, J=10.6, 4.5 Hz, 1H), 4.18-4.08 (m, 3H), 3.38-3.29 (m, 2H), 3.11 (br d, J=10.6 Hz, 1H), 2.72 (s, 1H), 1.07 (t, J=7.0 Hz, 1H), −0.02 (s, 1H). Analysis condition E: Retention time=1.54 min; ESI-MS(+) m/z [M+H]⁺: 439.0.

### Preparation of (S)-2-((((9H-fluoren-9-yl)methoxy)carbonyl)amino)-3-(isoquinolin-6-yl)propanoic acid

Step 1. In a 50-ml three neck flame-dried round bottom flask zinc (1.392 g, 21.28 mmol) was added under argon atmosphere and the flask was heated to 150° C. using a heat gun and was purged with argon. To the reaction DMF (30 mL) was added followed by the addition of 1,2-dibromoethane (10.48 μl, 0.12 mmol) and TMS-Cl (0.016 mL, 0.12 mmol) under argon. The reaction was stirred for 10 minutes. To the reaction mixture (R)-tert-butyl 2-((((9H-fluoren-9-yl)methoxy)carbonyl)amino)-3-iodopropanoate (3.0 g, 6.08 mmol) was added and the reaction was stirred for 1 hr To the reaction mixture 6-bromoisoquinoline (1.52 g, 7.30 mmol) and bis-(triphenylphosphino)-palladous chloride (0.20 g, 0.30 mmol) were added and the reaction was stirred for 16 h. The reaction mixture was diluted with ethyl acetate (50 mL), filtered through celite and washed with ethyl acetate (50 mL). The filtrate was concentrated under reduced pressure to afford the crude product as a red thick gum. The crude was purified by flash chromatography using 40 to 42% EtOAc in petroleum ether. After concentration on rotovap tert-butyl (S)-2-((((9H-fluoren-9-yl)methoxy)carbonyl)amino)-3-(isoquinolin-6-yl)propanoate (2.0 g, 66%) was obtained as a yellow gum. Analysis condition B: Retention time=2.46 min; ESI-MS(+) m/z [M+H]⁺: 495.3.

Step 2. The final product was obtained following the same procedure of (S)-2-((((9H-fluoren-9-yl)methoxy)carbonyl)amino)-3-(2-methyl-1H-indol-3-yl)propanoic acid. TFA hydrolysis with triethylsilane afforded the desired (S)-2-((((9H-fluoren-9-yl)methoxy)carbonyl)amino)-3-(isoquinolin-6-yl)propanoic acid as a grey solid in 90% yield after recrystallization in EtOAc and hexanes. ¹H NMR (400 MHz, METHANOL-d₄) δ 9.55 (s, 1H), 8.46 (d, J=6.5 Hz, 1H), 8.33 (d, J=8.5 Hz, 1H), 8.17 (d, J=6.0 Hz, 1H), 8.08 (s, 1H), 7.99-7.86 (m, 1H), 7.78 (dd, J=7.5, 4.0 Hz, 2H), 7.66-7.48 (m, 2H), 7.43-7.30 (m, 2H), 7.30-7.17 (m, 2H), 4.68 (dd, J=10.0, 4.5 Hz, 1H), 4.32-4.13 (m, 2H), 4.12-3.84 (m, 1H), 3.61 (dd, J=13.8, 4.8 Hz, 1H), 3.32-3.26 (m, 1H), 1.46 (s, 1H). Analysis condition B: Retention time=2.77 min; ESI-MS(+) m/z [M+H]⁺: 439.2.

### Preparation of (S)-2-((((9H-fluoren-9-yl)methoxy)carbonyl)amino)-3-(isoquinolin-4-yl)propanoic acid

Step 1. To a stirred mixture of zinc (2.319 g, 35.5 mmol) in DMF (50 mL) was added dibromomethane (0.071 mL, 1.014 mmol) and TMS-Cl (0.130 mL, 1.014 mmol). Exotherm was observed. The reaction mixture was stirred for 10 min. (R)-tert-butyl 2-((((9H-fluoren-9-yl)methoxy)carbonyl)amino)-3-iodopropanoate (5 g, 10.14 mmol) was added and again exotherm was observed. The reaction was allowed to stir for 1 h at room temperature. 2-Dicyclohexylphosphino-2′,6′-dimethoxybiphenyl (0.21 g, 0.51 mmol), tris(dibenzylideneacetone)dipalladium(0) (0.23 g, 0.25 mmol) and 4-bromoisoquinoline (2.11 g, 10.14 mmol) were added sequentially and the reaction was heated to 50° C. for 16 h. The reaction mixture was cooled to RT and treated with saturated ammonium chloride solution (200 mL). The crude was diluted with ethyl acetate (300 mL). Layers were separated and the organic layer was washed with brine and dried over anhydrous sodium sulphate. After filtration and concentration the crude product was purified by flash chromatography eluting with 30% of ethyl acetate in petroleum ether to afford tert-butyl (S)-2-((((9H-fluoren-9-yl)methoxy)carbonyl)amino)-3-(isoquinolin-4-yl)propanoate (2.5 g, 50%).

Analysis condition E: Retention time=3.44 min; ESI-MS(+) m/z [M+H]⁺: 495.2.

Step 2. The final product was obtained following the same procedure of (S)-2-((((9H-fluoren-9-yl)methoxy)carbonyl)amino)-3-(2-methyl-1H-indol-3-yl)propanoic acid. TFA hydrolysis afforded the desired (S)-2-((((9H-fluoren-9-yl)methoxy)carbonyl)amino)-3-(isoquinolin-4-yl)propanoic acid as an off white solid in quantitative yield after purification by diethyl ether trituration. ¹H NMR (400 MHz, DMSO-d₆) δ 9.55 (s, 1H), 8.52 (s, 1H), 8.44-8.24 (m, 2H), 8.18-8.00 (m, 1H), 7.95-7.80 (m, 4H), 7.59 (br d, J=7.5 Hz, 1H), 7.56 (br d, J=7.5 Hz, 1H), 7.47-7.34 (m, 2H), 7.34-7.24 (m, 2H), 4.46-4.30 (m, 1H), 4.25-4.02 (m, 3H), 3.69 (dd, J=14.1, 4.5 Hz, 1H), 3.37 (dd, J=14.1, 10.5 Hz, 1H), 0.10-0.11 (m, 1H). Analysis condition E: Retention time=1.57 min; ESI-MS(+) m/z [M+H]⁺: 441.2.

### Preparation of (S)-2-((((9H-fluoren-9-yl)methoxy)carbonyl)amino)-3-(4-(tert-butoxy)-3,5-difluorophenyl)propanoic acid

Step 1. The compound was prepared following the same procedure of tert-butyl (S)-2-((((9H-fluoren-9-yl)methoxy)carbonyl)amino)-3-(isoquinolin-4-yl)propanoate. First Negishi coupling with methyl (R)-2-((((9H-fluoren-9-yl)methoxy)carbonyl)amino)-3-iodopropanoate at 50° C. afforded the desired methyl (S)-2-((((9H-fluoren-9-yl)methoxy)carbonyl)amino)-3-(4-(tert-butoxy)-2,6-difluorophenyl)propanoate (5.5 g, 48.5% yield) after purification by flash chromatography.

Analysis condition E: Retention time=3.99 min; ESI-MS(+) m/z [M+NH₄]⁺: 527.2.

Step 2. In a multi-neck round bottom flask methyl (S)-2-((((9H-fluoren-9-yl)methoxy)carbonyl)amino)-3-(4-(tert-butoxy)-3,5-difluorophenyl)propanoate (11 g, 21.59 mmol) was added followed by the addition of tetrahydrofuran (132 mL) under nitrogen atmosphere at RT. The reaction mixture was cooled to 0° C. and LiOH (1.09 g, 45.3 mmol) in water (132 mL) solution was added. The reaction was stirred for 3 h. It was concentrated under reduced pressure below 38° C. to remove the solvent. The crude compound was cooled to 0° C., sat. Citric acid solution was added to adjust the pH to 4-5. It was extracted with ethyl acetate (3×250 mL). The combined organic layer was washed with water (200 mL) followed by brine (200 mL). The organic layer dried over sodium sulphate, filtered and concentrated under reduced pressure to give the crude (12 g) as a colorless thick mass. The crude compound was purified through ISCO using 120 g RediSep column, the product was eluted with 20% of ethyl acetate in petroleum ether. The reactions were concentrated to give (S)-2-((((9H-fluoren-9-yl)methoxy)carbonyl)amino)-3-(4-(tert-butoxy)-3,5-difluorophenyl)propanoic acid (9.0 g, 82%, HPLC purity 97%) as a white fluffy solid. Analysis condition E: Retention time=3.62 min; ESI-MS(+) m/z [M+H]⁺: 513.2. ¹H NMR (CDCl3, 400 MHz) δ 7.75 (d, J=7.6 Hz, 2H), 7.60 (m, 2H), 7.39 (t, J=7.6 Hz, 2H), 7.30 (m, 2H), 6.71 (d, J=7.6 Hz, 2H), 5.26 (m, 1H), 4.65 (m, 1H), 4.48-4.38 (m, 2H), 4.20 (m, 1H), 3.14-2.99 (m, 1H), 1.35 (s, 9H).

### Preparation of (S)-2-((((9H-fluoren-9-yl)methoxy)carbonyl)amino)-3-(isoquinolin-8-yl)propanoic acid

Step 1. Zinc (0.79 g, 12.00 mmol) was added to a flame-dried, nitrogen-purged side arm round-bottomed flask. DMF (5 mL) was added via syringe, followed by a catalytic amount of iodine (0.16 g, 0.63 mmol). A color change of the DMF was observed from colorless to yellow and back again. Protected (R)-tert-butyl 2-((((9H-fluoren-9-yl)methoxy)carbonyl)amino)-3-iodopropanoate (1.97 g, 4.00 mmol) was added immediately, followed by a catalytic amount of iodine (0.16 g, 0.63 mmol). The solution was stirred at room temperature; successful zinc insertion was accompanied by a noticeable exotherm. The solution of organozinc reagent was allowed to cool to room temperature and then Pd₂(dba)₃ (0.088 g, 0.096 mmol), dicyclohexyl(2′,6′-dimethoxy-[1,1′-biphenyl]-2-yl)phosphine (0.082 g, 0.200 mmol) and 8-bromoisoquinoline (1.082 g, 5.20 mmol) were added sequentially. The reaction mixture was stirred at 50 C for 4 h. under a positive pressure of nitrogen. The reaction mixture was cooled to RT, diluted with EtOAc (200 mL) and passed through Celite. The organic solvent was washed with sat. aq. NH₄Cl (200 mL), water (150 mL), and sat. aq. NaCl (200 mL), dried over Na₂SO₄, concentrated, and dried under vacuum to afford the crude compound. It was purified using ISCO combiflash column chromatography (24 g silica gel column, hexanes/ethyl acetate as the eluents) to afford (S)-tert-butyl 2-((((9H-fluoren-9-yl)methoxy)carbonyl)amino)-3-(isoquinolin-8-yl)propanoate (380 mg, 0.768 mmol, 19.21% yield). Analysis condition G: Retention time=2.59 min; ESI-MS(+) m/z [M+H]⁺: 495.3.

Step 2. (S)-tert-Butyl 2-((((9H-fluoren-9-yl)methoxy)carbonyl)amino)-3-(isoquinolin-8-yl)propanoate (380 mg, 0.768 mmol) was placed in 50-mi round bottom flask and was dissolved in DCM (8 mL). Triethylsilane (0.31 mL, 1.92 mmol) was added followed by trifluoroacetic acid (2.66 mL, 34.6 mmol). The reaction mixture was stirred at room temperature for 5 h. The solvents were evaporated, and the residue was dissolved in diethyl ether. The product was precipitated by the addition of petroleum ether. The resulting powder was then triturated with petroleum ether to yield (S)-2-((((9H-fluoren-9-yl)methoxy)carbonyl)amino)-3-(isoquinolin-8-yl)propanoic acid (320 mg, 0.712 mmol, 93% yield) as an off white solid. ¹H-NMR: (400 MHz, DMSO-d₆) δ ppm: 12.98 (bs, 1H), 9.79 (s, 1H), 8.62 (d, J=9.42 Hz, 1H), 8.22 (d, J=9.42 Hz, 1H), 8.06 (d, J=9.42 Hz, 1H), 7.84-7.93 (m, 4H), 7.74-7.76 (m, 1H), 7.56-7.58 (m, 1H), 7.38-7.42 (m, 2H), (m, 3H), 7.26-7.30 (m, 2H), 4.41 (m, 1H), 4.10-4.15 (m, 3H), 3.731-3.66 (m, 1H), 3.47-3.50 (m, 1H). Analysis condition G: Retention time=2.012 min; ESI-MS(+) m/z [M+H]⁺: 439.2 with 97.5% purity.

### Preparation of (S)-2-((((9H-fluoren-9-yl)methoxy)carbonyl)amino)-3-(7-fluoro-1H-indol-3-yl)propanoic acid

Step 1. Synthesis of tert-butyl 6-fluoro-3-iodo-1H-indole-1-carboxylate from 6-fluoro-1H-indole: A solution of iodine (3.76 g, 14.80 mmol) in DMF (15 mL) was dropped to the solution of 6-fluoro-1H-indole (2 g, 14.80 mmol) and potassium hydroxide (2.076 g, 37.0 mmol) in DMF (15 mL) at room temperature and the mixture was stirred for 45 min. The reaction mixture was then poured on 200 mL of ice water containing 0.5% ammonia and 0.1% sodium disulfite. The mixture was placed in a refrigerator to ensure the complete precipitation. The precipitate was filtered, washed with 100 mL ice water and dried in vacuo to obtain 3.80 g. The solid was suspended in dichloromethane (25 mL). 4-Dimethylaminopyridine (160 mg, 10 mol %) and di-tert-butyl dicarbonate (4.84 g, 22.20 mmol) were dissolved in dichloromethane (15 mL), and were added to the reaction. The resulting mixture was stirred for 30 min at room temperature, washed with 0.1 N HCl (25 mL) and the aqueous phase was extracted with dichloromethane (3×35 mL, monitored by TLC). The combined organic layers were dried with sodium sulfate, the solvents were removed under reduced pressure to obtain tert-butyl 6-fluoro-3-iodo-1H-indole-1-carboxylate (4.16 g, 11.52 mmol, 78% yield) as an orange solid. ¹H-NMR (CDCl₃) δ ppm: 7.82 (d, J=8.23 Hz, 1H), 7.68 (s 1H), 7.30-7.34 (m, 1H), 7.03-7.08 (m, 1H), 1.66 (s, 9H)

Step 2. Compound was prepared following the same procedure of (S)-tert-butyl 2-((((9H-fluoren-9-yl)methoxy)carbonyl)amino)-3-(isoquinolin-8-yl)propanoate. First Negishi coupling at 50° C. afforded the desired tert-butyl (S)-3-(2-((((9H-fluoren-9-yl)methoxy)carbonyl)amino)-3-(tert-butoxy)-3-oxopropyl)-7-fluoro-1H-indole-1-carboxylate (690 mg, 1.149 mmol, 57.4% yield) after purification by flash chromatography.

Analysis condition H: Retention time=3.885 min; ESI-MS(+) m/z [M-Boc-tBu+H]⁺: 445.2

Step 3. Final product was obtained following the same procedure of (S)-2-((((9H-fluoren-9-yl)methoxy)carbonyl)amino)-3-(isoquinolin-8-yl)propanoic acid. TFA hydrolysis afforded the desired (S)-2-((((9H-fluoren-9-yl)methoxy)carbonyl)amino)-3-(7-fluoro-1H-indol-3-yl)propanoic acid as an off white powder (96 mg, 0.191 mmol, 16.63% yield) after purification by reverse phase prep HPLC (Column: 80 g size, Silisep C18, 19×150 mm, 5 μm, Mobile phases: A=10 mM ammonium acetate in water, B=MeoH. 15 mL/min flow Gradient: 0-20 min, 5-30% B, 20-55 min, 30-80% B, 55-60 min, 80-100% B, held at 100% B for 5 min. Compound was eluted at 75% B) followed by lyophilization.

Analysis condition F: Retention time=1.367 min; ESI-MS(+) m/z [M+H]⁺: 445.3. ¹H-NMR (400 MHz, DMSO-d₆) δ ppm: 11.22 (s, 1H), 7.86 (d, J=8.72 Hz, 2H), 7.62-7.65 (m, 1H), 7.52-7.55 (m, 3H), 7.40-7.42 (m, 2H), 7.26-7.38 (m, 2H), 6.78-6.83 (m, 2H), 4.12-4.21 (m, 4H), 3.15-3.18 (m, 1H), 2.97-3.03 (m, 1H).

### Preparation of (2S,3S)-2-((((9H-fluoren-9-yl)methoxy)carbonyl)amino)-3-(1-(tert-butoxycarbonyl)-1H-indol-3-yl)butanoic acid

Compound (2S,3S)-2-azido-3-(1-(tert-butoxycarbonyl)-1H-indol-3-yl)butanoic acid was prepared following the procedure reported in Tetrahedron Letters 2001, 42, 4601-4603. The azide reduction step used different conditions as detailed below.

Step 1. To a solution of (2S,3S)-2-azido-3-(1-(tert-butoxycarbonyl)-1H-indol-3-yl)butanoic acid (1000 mg, 2.90 mmol) in THE (58 mL) was added platinum(IV) oxide (132 mg, 0.58 mmol). The reaction mixture was evacuated and filled with hydrogen. The reaction mixture was allowed to stir at room temperature with a hydrogen balloon for 2 h. The reaction mixture was evacuated and back filled with nitrogen three times. The solution was filtered through Celite©. The solvent was removed under vacuum and the crude residue was redissolved in EtOH. This solution was filtered through Celite© to give a clear solution which was concentrated under vacuum (0.89 g 96% yield). ¹H NMR (400 MHz, METHANOL-d₄) δ 8.13 (br d, J=8.0 Hz, 1H), 7.75 (d, J=7.8 Hz, 1H), 7.61 (s, 1H), 7.46-7.18 (m, 2H), 4.89 (s, 2H), 3.80 (d, J=6.5 Hz, 1H), 3.58 (t, J=7.2 Hz, 1H), 1.68 (s, 9H), 1.53 (d, J=7.3 Hz, 3H). Analysis condition B: Retention time=0.93 min; ESI-MS(+) m/z [M+H]⁺: 319.1.

Step 2. To a solution of (2S,3S)-2-amino-3-(1-(tert-butoxycarbonyl)-1H-indol-3-yl)butanoic acid (3.96 g, 12.44 mmol) in MeOH (25 mL) was added (9H-fluoren-9-yl)methyl 2,5-dioxopyrrolidine-1-carboxylate (888 mg, 2.76 mmol) followed by Et₃N (0.385 mL, 2.76 mmol). The reaction was stirred for 2 h at room temperature. The solvent was removed under vacuum and the residue was redissolved in EtOAc and washed with 1 N HCl aqueous solution then brine. The organic layer was collected, dried over anhydrous sodium sulfate, and concentrated under vacuum to give the desired product (1.3 g, 89% yield) which was not purified further. ¹H NMR (500 MHz, DMSO-d₆) δ 12.78 (br s, 1H), 8.07-7.80 (m, 2H), 7.76-7.48 (m, 4H), 7.46-7.15 (m, 6H), 5.75 (s, 1H), 4.44 (t, J=8.2 Hz, 1H), 4.33-4.22 (m, 1H), 4.19-4.07 (m, 2H), 1.56 (s, 9H), 1.39-1.27 (m, 3H). Analysis condition B: Retention time=1.27 min; ESI-MS(+) m/z [M+H]⁺: not observed.

### Preparation of (S)-2-((((9H-fluoren-9-yl)methoxy)carbonyl)amino)-3-(6-(o-tolyl)pyridin-3-yl)propanoic acid

Step 1. To a stirred solution of tert-butyl (S)-2-((((9H-fluoren-9-yl)methoxy)carbonyl)amino)-3-(6-bromopyridin-3-yl)propanoate (1750 mg, 3.35 mmol) in toluene/iPrOH (1:1, v:v, 50 mL) was added o-tolylboronic acid (911.6 mg, 6.7 mmol) and 2M Na₂CO₃ aqueous solution (25.0 mL). The mixture was purged with argon three times. Dichlorobis(tricyclohexylphosphine)palladium(II) (123.6 mg, 0.167 mmol) was added and the reaction mixture was purged twice with argon. The reaction was heated to 80° C. for 20 h. The reaction was cooled to room temperature and iPrOH was removed by rotovap. The crude was partitioned between water and EtOAc. The aqueous phase was extracted with EtOAc. Organic phases were combined and dried over anhydrous MgSO₄. After filtration and concentration the crude product was obtained as a brown oil. Purification by flash chromatography using EtOAc:DCM (1:9) as eluant lead to tert-butyl (S)-2-((((9H-fluoren-9-yl)methoxy)carbonyl)amino)-3-(6-(o-tolyl)pyridin-3-yl)propanoate (1.81 g, 3.39 mmol, 90%) as a colorless oil.

Step 2. (S)-2-((((9H-Fluoren-9-yl)methoxy)carbonyl)amino)-3-(6-(o-tolyl)pyridin-3-yl)propanoate (1750 mg, 3.19 mmol) was dissolved in trifluoroacetic acid (5.00 mL) and the reaction was allowed to stir at room temperature for two hours. The reaction was brought to dryness on a rotovap and the crude product was dissolved in diethyl ether and 1M HCl in diethyl ether. The mixture was sonicated for 2 hours to give a white solid. The product was isolated by filtration and washed with water to give (S)-2-((((9H-fluoren-9-yl)methoxy)carbonyl)amino)-3-(6-(o-tolyl)pyridin-3-yl)propanoic acid (1.91 g, 3.99 mmol, 100%) as a white solid. ¹H NMR (499 MHz, DMSO-d₆) δ 8.90 (s, 1H), 8.48 (br d, J=8.0 Hz, 1H), 7.96 (t, J=6.9 Hz, 2H), 7.89 (d, J=7.5 Hz, 2H), 7.64 (dd, J=7.2, 4.8 Hz, 2H), 7.52-7.45 (m, 1H), 7.43-7.29 (m, 7H), 4.46 (ddd, J=10.7, 8.9, 4.5 Hz, 1H), 4.25-4.15 (m, 3H), 3.45-3.34 (m, 1H), 3.18-3.10 (m, 1H), 3.08-3.00 (m, 1H), 2.27-2.20 (m, 3H).

### Preparation of (S)-2-((((9H-fluoren-9-yl)methoxy)carbonyl)amino)-3-(4′-acetamido-[1,1′-biphenyl]-4-yl)propanoic acid

Step 1. A 5.0-1 multi-neck round-bottomed flask was charged with (S)-2-amino-3-(4-bromophenyl)propanoic acid (150.0 g, 615 mmol), Fmoc-OSu (207 g, 615 mmol) in acetone (1500 mL), a solution of sodium bicarbonate (258 g, 3073 mmol) in water (3000 mL) in one lot and allowed to stir at room temperature for 16 h. The reaction mixture was slowly acidified with 10 N HCl solution to pH 1 and stirred for 15 min. The slurry was filtered and dried under vacuum and the cake was washed with water (3.0 L). Solids were dried for 16 h. The desired product was obtained as a white solid (280 g, 98%) and the product was taken to the next stage. Analysis condition E: Retention time=2.17 min; ESI-MS(+) m/z [M+H]⁺: 466.2.

Step 2. To a stirred solution of (S)-2-((((9H-fluoren-9-yl)methoxy)carbonyl)amino)-3-(4-bromophenyl)propanoic acid (1.0 g, 2.144 mmol) and (4-acetamidophenyl)boronic acid (0.576 g, 3.22 mmol) in THE (50 mL) in a 150-ml pressure tube, Argon was purged for 5 min. Potassium phosphate, tribasic (1.366 g, 6.43 mmol) was then added and the purging was continued for another 5 min. 1,1′-bis(di-tert-butylphosphino)ferrocene palladium dichloride (0.140 g, 0.214 mmol) was then added, and the purging was continued for another 5 min. The reaction mixture was heated to 65° C. for 26 h. The reaction mass was diluted with EtOAc (25 mL) and washed with 10% citric acid aqueous solution (10 mL) and then brine solution to provide the crude product. It was triturated with 20% DCM, stirred for 10 min and filtered with a buchner funnel, and then dried for 10 min. The crude was purified by flash chromatography to give 0.7 g (57%) of the desired product as a brown solid. Analysis condition E: Retention time=1.79 min; ESI-MS(+) m/z [M+H]⁺: 519.0. ¹H NMR (400 MHz, DMSO-d₆) δ 12.75 (br s, 1H), 9.99 (s, 1H), 7.87 (d, J=7.5 Hz, 2H), 7.77-7.49 (m, 9H), 7.47-7.22 (m, 7H), 4.26-4.13 (m, 4H), 3.11 (br dd, J=13.8, 4.3 Hz, 1H), 2.91 (dd, J=13.8, 10.8 Hz, 1H), 2.12-2.01 (m, 4H).

### Synthesis of Aryl/Heteroaryl Substituted Phenylalanines

General procedures for Suzuki-Miyaura coupling (SMC) reactions in Scheme 1. To a N₂-flushed 20-mL scintillation vial equipped with a magnetic stir bar was added Fmoc-halo-Phe-OH (0.5 mmol), boronic acid (1.5-2.5 equiv.), and anhydrous THE (6 mL). The suspension was degassed by bubbling N₂ into the vial for several minutes. Palladium(II) acetate (4.5 mol %), DtBuPF (5 mol %), and then anhydrous K₃PO₄ (2.5 equiv.) were added. The suspension was degassed for several minutes, and then the vial was capped with a septum. The reaction mixture was stirred at 50° C. for 16 h. After cooling, 20% aqueous citric acid solution was added to acidify the reaction. The organic layer was separated, and the aqueous layer was extracted with EtOAc (2×). Silica gel was added to the combined organic layers, and the mixture was concentrated to dryness. The residue was dry-loaded on a silica gel column (ISCO system) and eluted with hexanes/EtOAc to give the desired product. Sometimes for compounds which are tailing in a Hexanes/EtOAc system, further eluting with MeOH/CH₂Cl₂ is also needed.

### Preparation of (S)-2-((((9H-fluoren-9-yl)methoxy)carbonyl)amino)-3-(4′-(tert-butoxycarbonyl)-[1,1′-biphenyl]-4-yl)propanoic acid

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 78% (439 mg); colorless solids. ¹H NMR (400 MHz, methanol-d₄) δ 7.94 (d, J=8.3 Hz, 2H), 7.74 (d, J=7.6 Hz, 2H), 7.56 (d, J=8.4 Hz, 4H), 7.51 (d, J=8.1 Hz, 2H), 7.38-7.28 (m, 4H), 7.28-7.17 (m, 2H), 4.56-4.38 (m, 1H), 4.29 (dd, J=10.5, 7.0 Hz, 1H), 4.17 (dd, J=10.5, 7.1 Hz, 1H), 4.08 (t, J=7.0 Hz, 1H), 3.29-3.21 (m, 1H), 2.98 & 2.80 (dd, J=13.8, 9.6 Hz, total 1H), 1.59 (s, 9H). ESI-HRMS: Calcd for C₃₅H₃₄NO₆ [M+H]⁺ 564.23806, found 564.23896, mass difference 1.588 ppm.

### Preparation of (S)-2-((((9H-fluoren-9-yl)methoxy)carbonyl)amino)-3-(3′-(tert-butoxycarbonyl)-[1,1′-biphenyl]-4-yl)propanoic acid

(S)-2-((((9H-Fluoren-9-yl)methoxy)carbonyl)amino)-3-(4′-(tert-butoxycarbonyl)-[1,1′-biphenyl]-4-yl)propanoic acid was prepared according to the SMC general procedure. Yield: 85% (240 mg); off-white solids. ¹H NMR (500 MHz, DMSO-d₆) δ 8.08 (t, J=1.8 Hz, 1H), 7.86 (dd, J=7.7, 1.4 Hz, 3H), 7.83 (d, J=8.1 Hz, 1H), 7.64 (d, J=7.7 Hz, 1H), 7.63 (d, J=7.5 Hz, 1H), 7.58-7.48 (m, 3H), 7.41-7.35 (m, 2H), 7.31 (d, J=7.8 Hz, 2H), 7.30-7.23 (m, 2H), 4.31-4.10 (m, 4H), 4.05 (td, J=8.2, 4.5 Hz, 1H), 3.13 & 2.9 (dd, J=13.6, 4.5 Hz, total 1H), 2.94 & 2.76 (dd, J=13.6, 8.7 Hz, total 1H), 1.56 (s, 9H). ESI-HRMS: Calcd for C₃₅H₃₇N₂O₆ [M+NH₄]⁺ 581.26461, found at 581.26474, mass difference 0.218 ppm.

### Preparation of (S)-2-((((9H-fluoren-9-yl)methoxy)carbonyl)amino)-3-(4-boronophenyl)propanoic acid (ELN: A0934-595-01)

To a 75-ml pressure bottle (S)-2-((((9H-fluoren-9-yl)methoxy)carbonyl)amino)-3-(4-bromophenyl)propanoic acid (6.0 g, 12.87 mmol) and 2-methyl THE (250 mL) were charged, and the solution was purged with argon for 5 min. Tri-o-tolylphosphine (0.31 g, 1.03 mmol), tetrahydroxydiboron (2.31 g, 25.7 mmol), potassium acetate (3.79 g, 38.6 mmol) were added in 10-min interval followed by the addition of MeOH (100 mL) and Pd(OAc)₂ (0.12 g, 0.52 mmol), and argon was purged for 10 min. The reaction was heated at 50° C. overnight. The reaction mixture was transferred into a 1-liter separatory funnel, diluted with 2-methyl-THF, and acidified with 1.5 N HCl to pH=2. The organic layer was washed with brine, dried (sodium sulphate), passed through celite, and concentrated to give black crude material. The crude was treated with petroleum ether to give a solid (10 g) which was dissolved with 2-methyl-THF and charcoal (2 g) was added. The mixture was heated on a rotovap without vacuum at 50° C. After filtration, the filtrate was passed through celite, and concentrated. The resulting solid was treated with 30% ethyl acetate in petroleum ether, filtered to give 8 g of the crude as a fine off-white solid, which was further purified via flash chromatography then trituration with petroleum ether to give (S)-2-((((9H-fluoren-9-yl)methoxy)carbonyl)amino)-3-(4-boronophenyl)propanoic acid (4.0 g, 9.28 mmol, 72.1% yield) as a white solid. LCMS: 432.1 (M+H), tr=0.82 min. ¹H NMR (500 MHz, DMSO-d₆) δ 7.88 (d, J=7.6 Hz, 2H), 7.85-7.77 (m, 1H), 7.71 (br d, J=7.9 Hz, 3H), 7.68-7.60 (m, 2H), 7.41 (br d, J=6.6 Hz, 2H), 7.35-7.20 (m, 4H), 4.30-4.11 (m, 5H), 3.16-3.03 (m, 1H), 2.95-2.83 (m, 1H).

### Preparation of (S)-2-((((9H-fluoren-9-yl)methoxy)carbonyl)amino)-3-(4′-fluoro-[1,1′-biphenyl]-4-yl)propanoic acid

To a stirred solution of (S)-2-((((9H-fluoren-9-yl)methoxy)carbonyl)amino)-3-(4-boronophenyl)propanoic acid (217.5 mg, 0.504 mmol), 1-bromo-4-fluorobenzene (0.083 mL, 0.757 mmol) and XPhos Pd G2 (9.7 mg, 0.012 mmol) in THE (1 mL) at RT was added 0.5 M aqueous K₃PO₄ (2 mL, 1.000 mmol). N₂ was purged with vacuum three times and the mixture was stirred at 80° C. for 16 h. The mixture was cooled to RT. To the reaction was added 10% citric acid until pH<6. It was partitioned between EtOAc and H₂O, and the organic phase was separated, washed with brine, and dried over sodium sulfate. The mixture was filtered, SiO₂ (5 g) was added and concentrated. The material was then purified by flash chromatography (Teledyne ISCO CombiFlash R_{f}, gradient of 0% to 20% MeOH/CH₂Cl₂ over 15 column volumes, RediSep SiO₂ 40 g). Fractions containing the desired product were collected and concentrated to give (S)-2-((((9H-fluoren-9-yl)methoxy)carbonyl)amino)-3-(4′-fluoro-[1,1′-biphenyl]-4-yl)propanoic acid (206.1 mg, 0.43 mmol, 85% yield) as a cream solid: HPLC: RT=1.04 min (Waters Acquity UPLC BEH C18 1.7 um 2.1×50 mm, CH₃CN/H₂O/0.05% TFA, 1 min. gradient, wavelength=254 nm); MS (ES): m/z=482 [M+H]⁺. ¹H NMR (499 MHz, DMSO-d₆) δ 12.78 (br s, 1H), 7.88 (d, J=7.5 Hz, 3H), 7.71-7.61 (m, 5H), 7.53 (d, J=8.1 Hz, 2H), 7.39 (q, J=7.3 Hz, 3H), 7.36-7.23 (m, 8H), 4.24-4.13 (m, 5H), 3.12 (dd, J=14.0, 4.5 Hz, 1H), 2.91 (dd, J=13.6, 10.3 Hz, 1H).

### Preparation of (S)-2-((((9H-fluoren-9-yl)methoxy)carbonyl)amino)-3-(3′,5′-difluoro-[, 1′-biphenyl]-4-yl)propanoic acid

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,5′-difluoro-[1,1′-biphenyl]-4-yl)propanoic acid (197.1 mg, 0.40 mmol, 78% yield) as a colorless solid after purification by flash chromatography. HPLC: RT=1.06 min (Waters Acquity UPLC BEH C18 1.7 um 2.1×50 mm, CH₃CN/H₂O/0.05% TFA, 1 min. gradient, wavelength=254 nm); MS (ES): m/z=500 [M+H]⁺. ¹H NMR (499 MHz, DMSO-d₆) δ 12.90-12.67 (m, 1H), 7.87 (d, J=7.5 Hz, 2H), 7.69-7.61 (m, 4H), 7.45-7.35 (m, 6H), 7.33-7.27 (m, 2H), 7.22-7.16 (m, 1H), 4.25-4.18 (m, 3H), 4.17-4.12 (m, 1H), 3.14 (dd, J=13.8, 4.4 Hz, 1H), 2.92 (dd, J=13.7, 10.6 Hz, 1H).

### Preparation of (S)-2-((((9H-fluoren-9-yl)methoxy)carbonyl)amino)-3-(3′,4′,5′-trifluoro-[1′-biphenyl]-4-yl)propanoic acid

The final product was obtained following the same procedure of (S)-2-((((9H-fluoren-9-yl)methoxy)carbonyl)amino)-3-(4′-fluoro-[1,1′-biphenyl]-4-yl)propanoic acid. The Suzuki coupling reaction afforded the desired (S)-2-((((9H-fluoren-9-yl)methoxy)carbonyl)amino)-3-(3′,4′,5′-trifluoro-[1,1′-biphenyl]-4-yl)propanoic acid (218.5 mg, 0.422 mmol, 84% yield) as a colourless solid after purification by flash chromatography. HPLC: RT=1.466 min (Shimadzu UPLC with Waters Acquity BEH C18 1.7 um 2.1×50 mm column, CH₃CN/H₂O/0.1% TFA, 3 min. gradient, wavelength=254 nm); MS (ES): m/z=556. ¹H NMR (499 MHz, DMSO-d₆) δ 12.79 (br s, 1H), 7.87 (d, J=7.6 Hz, 2H), 7.75 (d, J=8.6 Hz, 1H), 7.69-7.58 (m, 6H), 7.44-7.35 (m, 4H), 7.33-7.25 (m, 2H), 4.27-4.17 (m, 3H), 4.17-4.10 (m, 1H), 3.14 (dd, J=13.8, 4.4 Hz, 1H), 2.92 (dd, J=13.7, 10.7 Hz, 1H).

General procedure for photoredox reaction. Ir[dF(CF3)ppy₂]₂(dtbbpy)PF₆ (0.018 g, 0.016 mmol, 1 mol %), tert-butyl (R)-2-((((9H-fluoren-9-yl)methoxy)carbonyl)amino)-3-iodopropanoate (1.181 g, 2.393 mmol, 1.5 equiv), bromo-pyridine derivative (1.596 mmol, 1.00 equiv), pulverized Na₂CO₃ (0.338 g, 3.19 mmol, 2.00 equiv), and tris(trimethylsilane)silane (0.278 g, 1.596 mmol, 1.00 equiv) were charged into an oven-dried 40-mL pressure-relief screw cap vial. The vial was capped, purged with nitrogen, diluted with THF (45.0 mL), and then sonicated. In a separate vial were charged NiCl₂-glyme (18 mg, 0.080 mmol, 5 mol %) and di-tertbutylbipyridine (18 mg, 0.096 mmol, 6 mol %) in 1 mL dioxane. The vial was purged with nitrogen for 10 min. The Nickel-ligand complex solution was transferred to the main reaction vial and the mixture was degassed with gentle nitrogen flow for 20 min. The reactor was sealed with parafilm and placed between 2 34 W blue LED Kessil lamps (ca. 7 cm away) and allowed to stir vigorously. After 16 h, the reaction was monitored by LCMS analysis. The resulting oil was dissolved into 4 M HCl dioxane solution (15 mL). After 16 h, the reaction mixture was brought to dryness on a rotovap. The crude product was dissolved in a minimum amount of methanol and dry loaded on a silica gel column for purification.

### Preparation of (2S)-2-({[(9H-fluoren-9-yl)methoxy]carbonyl}amino)-3-(2-methoxypyridin-4-yl)propanoic acid

The mixture was rotovaped onto silica gel, purified by ISCO using 10% to 80% EtOAc/Hexanes. The fractions were pooled, concentrated to obtain the desired product as a clear oil (237 mg, 100%)

Analysis conditions D: Retention time 1.74 min; ES+ 475.1.

### Preparation of ((S)-2-((((9H-fluoren-9-yl)methoxy)carbonyl)amino)-3-(4-(trifluoromethoxy)phenyl)propanoic acid

Step 1. In 4 separate 40-ml vials was placed Ir(dF(CF3)ppy)₂(dtbbpy)PF₆ (5.6 mg, 4.99 μmol) and Na₂CO₃ (249 mg, 2.35 mmol) in dioxane (18 mL), and each vial was fitted with a teflon screw cap and a stir bar. To the mixture was added 1-iodo-4-(trifluoromethoxy)benzene (0.16 mL, 1.02 mmol), stirred briefly, then tris(trimethylsilyl)silane (0.23 mL, 0.75 mmol) was added via syringe, and the suspension was degassed (cap on) with nitrogen for 5 min. To a separate 40-μmL vial was added nickel(II) chloride ethylene glycol dimethyl ether complex (22 mg, 0.10 mmol) and 4,4′-di-tert-butyl-2,2′-bipyridine (33 mg, 0.12 mmol). Dioxane (10 mL) was added and this solution was degassed (cap on) with nitrogen gas for 10 min and stirred. To the Ir mixture was added 2.5 mL of the Ni solution, and 5 mL of a solution of the iodo alanine, tert-butyl (R)-2-((((9H-fluoren-9-yl)methoxy)carbonyl)amino)-3-iodopropanoate (987 mg, 2.0 mmol) in dioxane (20 mL), and then the mixture was further degassed with nitrogen gas for another 5 min (cap on). The vials were sealed with parafilm, placed in the round photoredox reactor with light and fan on, and stirred for 40 h. The reactions were removed from the illumination/reactor. The blackish reaction mixtures of each vial were poured into a 500-ml erlenmeyer flask into which was added EtOAc (200 mL). The mixture was filtered through celite, washed with EtOAc, and concentrated. The residue was purified by flash chromatography (Teledyne ISCO CombiFlash R_{f}, gradient of 0% using solvent A/B=CH₂Cl₂/EtOAc over 10 column volumes, RediSep SiO₂ 80 g, loaded as DCM solution). The fractions containing the desired product were collected and concentrated to obtained the product tert-butyl (S)-2-((((9H-fluoren-9-yl)methoxy)carbonyl)amino)-3-(4-(trifluoromethoxy)phenyl)propanoate (865.2 mg, 1.64 mmol, 82% yield, only about 73% HPLC purity as a colourless oil and was used as is in the deprotection step: HPLC: RT=1.62 min (Waters Acquity UPLC BEH C18 1.7 um 2.1×50 mm, CH₃CN/H₂O/0.05% TFA, 1 min. gradient, wavelength=254 nm); MS (ES): m/z=550 [M+23]+

Step 2. To a stirred solution of tert-butyl (S)-2-((((9H-fluoren-9-yl)methoxy)carbonyl)amino)-3-(4-(trifluoromethoxy)phenyl)propanoate (865.2 mg, 1.64 mmol) in dichloromethane (8.2 mL) at RT was added HCl (4M in dioxane, 8.20 mL, 32.8 mmol). The mixture was stirred at RT for 18 h. The mixture was concentrated in vacuo then dried under vacuum. The residue was dissolved in DMF (4 mL), and purified on an ISCO ACCQ Prep over 2 injections. The fractions containing the desired product were combined and partially concentrated on a rotovap, then air was blown over the mixture over the weekend. The residue was dissolved in CH₃CN, diluted with water, frozen, and lyophilized to obtain the product (S)-2-((((9H-fluoren-9-yl)methoxy)carbonyl)amino)-3-(4-(trifluoromethoxy)phenyl)propanoic acid (344.1 mg, 0.73 mmol, 44.5% yield) as a colorless solid. HPLC: RT=1.38 min (Waters Acquity UPLC BEH C18 1.7 um 2.1×50 mm, CH₃CN/H₂O/0.05% TFA, 1.5 min. gradient, wavelength=254 nm); MS (ES): m/z=472 [M+1]+

¹H NMR (499 MHz, DMSO-d₆) ppm δ 7.88 (d, J=7.5 Hz, 2H), 7.63 (d, J=7.4 Hz, 2H), 7.44-7.37 (m, 2H), 7.35-7.25 (m, 4H), 7.19 (br d, J=7.6 Hz, 3H), 4.30-4.20 (m, 1H), 4.21-4.13 (m, 2H), 4.04 (br d, J=3.5 Hz, 1H), 3.11 (br dd, J=13.6, 4.4 Hz, 1H), 2.91 (br dd, J=13.6, 9.1 Hz, 1H).

### Preparation of (S)-2-((((9H-fluoren-9-yl)methoxy)carbonyl)amino)-3-(2,5-dimethylphenyl)propanoic acid

Step 1. Compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,5-dimethylphenyl)propanoate (140.5 mg, 0.298 mmol, 61.1% yield) after purification by flash chromatography. Analysis condition J: Retention time=1.21 min; ESI-MS(+) m/z [M-tBu+H]⁺: 416. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.07 (d, J=7.7 Hz, 1H), 6.98 (d, J=7.7 Hz, 1H), 6.96 (s, 1H), 4.58-4.51 (m, 1H), 4.39 (dd, J=10.5, 7.3 Hz, 1H), 4.34 (dd, J=10.5, 7.2 Hz, 1H), 4.24-4.19 (m, 1H), 3.10-3.01 (m, 2H), 2.34 (s, 3H), 2.28 (s, 3H), 1.40 (s, 8H)

Step 2. Final product was obtained following the same procedure of (S)-2-((((9H-fluoren-9-yl)methoxy)carbonyl)amino)-3-(4-(trifluoromethoxy)phenyl)propanoic acid. Removal of tBu ester with HCl/dioxane afforded the desired (S)-2-((((9H-fluoren-9-yl)methoxy)carbonyl)amino)-3-(2,5-dimethylphenyl)propanoic acid (115.2 mg, 0.277 mmol, 93% yield) as a cream solid after purification by reverse phase flash chromatography. Analysis condition J: RT=1.03 min, MS (ES): m/z=416 [M+H]⁺. ¹H NMR (499 MHz, CHLOROFORM-d) δ 7.88 (d, J=7.4 Hz, 2H), 7.79 (br d, J=8.6 Hz, 1H), 7.67 (d, J=7.4 Hz, 1H), 7.64 (d, J=7.5 Hz, 1H), 7.41 (td, J=7.3, 4.2 Hz, 3H), 7.35-7.29 (m, 2H), 7.29-7.25 (m, 1H), 7.02 (br d, J=8.9 Hz, 2H), 6.91 (br d, J=7.4 Hz, 1H), 4.21-4.10 (m, 5H), 3.07 (dd, J=14.1, 4.4 Hz, 1H), 2.80 (dd, J=14.1, 10.3 Hz, 1H), 2.24 (s, 3H), 2.18 (s, 3H)

### Preparation of (S)-2-((((9H-fluoren-9-yl)methoxy)carbonyl)amino)-3-(4-fluoro-3-methylphenyl)propanoic acid

Step 1. The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(4-fluoro-3-(trifluoromethyl)phenyl)propanoate (66.3 mg, 0.13 mmol, 24.9% yield) as a colourless solid after purification by flash chromatography. HPLC: RT=1.19 min (Waters Acquity UPLC BEH C18 1.7 um 2.1×50 mm, CH₃CN/H₂O/0.05% TFA, 1 min. gradient, wavelength=254 nm); MS (ES): m/z=474 [M-tBu]*. ¹H NMR (499 MHz, CHLOROFORM-d) δ 7.80 (d, J=7.5 Hz, 2H), 7.60 (dd, J=7.6, 3.3 Hz, 2H), 7.47-7.39 (m, 3H), 7.38-7.32 (m, 2H), 7.16-7.09 (m, 1H), 5.34 (br d, J=7.7 Hz, 1H), 4.57-4.47 (m, 2H), 4.40 (dd, J=10.3, 6.9 Hz, 1H), 4.26-4.21 (m, 1H), 3.14 (br d, J=4.9 Hz, 2H), 1.44 (s, 9H)

Step 2. Final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(4-fluoro-3-methylphenyl)propanoic acid (58.3 mg, 0.139 mmol, 85% yield) as a cream solid after purification by reverse phase flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=420 [M+H]⁺. ¹H NMR (499 MHz, DMSO-d₆) δ 12.86-12.66 (m, 1H), 7.89 (d, J=7.5 Hz, 2H), 7.73 (d, J=8.3 Hz, 1H), 7.65 (t, J=7.5 Hz, 2H), 7.42 (t, J=7.5 Hz, 2H), 7.35-7.26 (m, 2H), 7.17 (br d, J=7.5 Hz, 1H), 7.14-7.08 (m, 1H), 7.06-6.99 (m, 1H), 4.24-4.11 (m, 4H), 3.03 (dd, J=13.7, 4.3 Hz, 1H), 2.82 (dd, J=13.6, 10.6 Hz, 1H), 2.17 (s, 3H).

### Preparation of (S)-2-((((9H-fluoren-9-yl)methoxy)carbonyl)amino)-3-(2,4-difluoro-5-methoxyphenyl)propanoic acid

Step 1. The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,4-difluoro-5-methoxyphenyl)propanoate (77.1 mg, 0.151 mmol, 29.1% yield as a colourless solid after purification by flash chromatography. HPLC: RT=1.15 min (Waters Acquity UPLC BEH C18 1.7 um 2.1×50 mm, CH₃CN/H₂O/0.05% TFA, 1 min. gradient, wavelength=254 nm); MS (ES): m/z=454 [M-t-Bu]⁺. ¹H NMR (499 MHz, CHLOROFORM-d) δ 7.79 (d, J=7.4 Hz, 2H), 7.59 (t, J=6.4 Hz, 2H), 7.43 (t, J=7.3 Hz, 2H), 7.33 (td, J=7.5, 1.1 Hz, 3H), 6.85 (dd, J=10.8, 9.3 Hz, 1H), 6.83-6.79 (m, 1H), 5.40 (br d, J=8.1 Hz, 1H), 4.58-4.51 (m, 1H), 4.38 (dd, J=7.0, 4.5 Hz, 2H), 4.25-4.20 (m, 1H), 3.82 (s, 3H), 3.18-3.05 (m, 2H), 1.45 (s, 9H).

Step 2. The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2,4-difluoro-5-methoxyphenyl)propanoic acid (45.9 mg, 0.101 mmol, 66.9% yield) as a cream solid after purification by reverse phase flash chromatography. HPLC: RT=0.99 min (Waters Acquity UPLC BEH C18 1.7 um 2.1×50 mm, CH₃CN/H₂O/0.05% TFA, 1 min. gradient, wavelength=254 nm); MS (ES): m/z=454 [M+1]⁺. ¹H NMR (499 MHz, DMSO-d₆) δ 12.92 (br s, 1H), 7.89 (d, J=7.5 Hz, 2H), 7.71-7.65 (m, 1H), 7.63 (d, J=7.5 Hz, 2H), 7.41 (t, J=7.5 Hz, 2H), 7.34-7.25 (m, 2H), 7.24-7.15 (m, 2H), 4.24-4.12 (m, 4H), 3.77 (s, 3H), 3.16 (br dd, J=13.8, 4.6 Hz, 1H), 2.82 (dd, J=13.6, 10.7 Hz, 1H).

### Preparation of (S)-2-((((9H-fluoren-9-yl)methoxy)carbonyl)amino)-3-(2,3-dimethylphenyl)propanoic acid

Step 1. The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2,3-dimethylphenyl)propanoate (107.5 mg, 0.228 mmol, 55.5% yield) as a tan viscous oil after purification by flash chromatography. HPLC: RT=1.21 min (Waters Acquity UPLC BEH C18 1.7 um 2.1×50 mm, CH₃CN/H₂O/0.05% TFA, 1 min. gradient, wavelength=254 nm); MS (ES): m/z=416 [M-t-Bu]⁺. ¹H NMR (499 MHz, CHLOROFORM-d) δ 7.79 (d, J=7.5 Hz, 2H), 7.61-7.56 (m, 2H), 7.42 (t, J=7.5 Hz, 2H), 7.35-7.31 (m, 2H), 7.09-7.06 (m, 1H), 7.02 (t, J=7.5 Hz, 1H), 7.00-6.96 (m, 1H), 5.30 (br d, J=8.3 Hz, 1H), 4.53 (q, J=7.4 Hz, 1H), 4.39 (dd, J=10.6, 7.3 Hz, 1H), 4.34 (dd, J=10.4, 7.0 Hz, 1H), 4.21 (t, J=7.2 Hz, 1H), 3.15 (dd, J=14.2, 7.0 Hz, 1H), 3.08 (dd, J=14.1, 7.3 Hz, 1H), 2.29 (s, 3H), 2.28 (s, 3H), 1.40 (s, 9H).

Step 2. The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2,3-dimethylphenyl)propanoic acid (72.9 mg, 0.175 mmol, 77% yield) as a cream solid after purification by reverse phase flash chromatography. HPLC: RT=1.03 min (Waters Acquity UPLC BEH C18 1.7 um 2.1×50 mm, CH₃CN/H₂O/0.05% TFA, 1 min. gradient, wavelength=254 nm); MS (ES): m/z=416 [M+H]⁺. ¹H NMR (499 MHz, DMSO-d₆) δ 12.76 (br d, J=1.8 Hz, 1H), 7.89 (d, J=7.5 Hz, 2H), 7.79-7.71 (m, 1H), 7.66 (dd, J=13.6, 7.6 Hz, 2H), 7.42 (td, J=7.2, 4.1 Hz, 2H), 7.35-7.27 (m, 2H), 7.07 (d, J=7.3 Hz, 1H), 7.04-6.99 (m, 1H), 6.99-6.94 (m, 1H), 4.24-4.14 (m, 3H), 4.13-4.05 (m, 1H), 3.15 (dd, J=14.1, 4.1 Hz, 1H), 2.85 (dd, J=13.9, 10.4 Hz, 1H), 2.22 (s, 3H), 2.19 (s, 3H).

### Preparation of (S)-2-((((9H-fluoren-9-yl)methoxy)carbonyl)amino)-3-(2-fluoro-3-methylphenyl)propanoic acid

Step 1. The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-3-methylphenyl)propanoate (136.9 mg, LCMS showed 77% product and 23% impurity) as a viscous oil after purification by flash chromatography. Used as is; purified after tBu hydrolysis.

Step 2. The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2-fluoro-3-methylphenyl)propanoic acid (79.7 mg, 0.190 mmol, 66.0% yield) as a cream solid after purification by reverse phase flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=420 [M+1]⁺. ¹H NMR (499 MHz, DMSO-d₆) δ 12.79 (br s, 1H), 7.89 (d, J=7.7 Hz, 2H), 7.78 (d, J=8.6 Hz, 1H), 7.65 (dd, J=11.6, 7.5 Hz, 2H), 7.44-7.39 (m, 3H), 7.37-7.25 (m, 3H), 7.14 (br t, J=7.4 Hz, 2H), 7.01-6.96 (m, 1H), 4.24-4.12 (m, 4H), 3.17 (dd, J=13.8, 4.8 Hz, 1H), 2.86 (dd, J=13.6, 10.8 Hz, 1H), 2.21 (s, 3H). ¹H NMR and LCMS showed a 14% impurity.

### Preparation of ((S)-2-((((9H-fluoren-9-yl)methoxy)carbonyl)amino)-3-(2-fluoro-5-methylphenyl)propanoic acid

The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-fluoro-5-methylphenyl)propanoate (148.1 mg, 0.311 mmol, 65.4% yield) as a colourless gum after purification by flash chromatography. HPLC: RT=1.19 min (Waters Acquity UPLC BEH C18 1.7 um 2.1×50 mm, CH₃CN/H₂O/0.05% TFA, 1 min. gradient, wavelength=254 nm); MS (ES): m/z=420 [M-t-Bu]⁺. ¹H NMR (499 MHz, CHLOROFORM-d) δ 7.79 (d, J=7.6 Hz, 2H), 7.60 (t, J=7.2 Hz, 2H), 7.42 (t, J=7.4 Hz, 2H), 7.37-7.30 (m, 2H), 7.06-6.99 (m, 2H), 6.97-6.90 (m, 1H), 5.41 (br d, J=8.1 Hz, 1H), 4.60-4.54 (m, 1H), 4.43 (dd, J=10.4, 7.2 Hz, 1H), 4.30 (dd, J=10.1, 7.5 Hz, 1H), 4.26-4.21 (m, 1H), 3.16 (dd, J=13.9, 6.7 Hz, 1H), 3.10 (dd, J=13.9, 6.4 Hz, 1H), 2.28 (s, 3H), 1.44 (s, 9H).

Step 2. The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2-fluoro-5-methylphenyl)propanoic acid (98.1 mg, 0.23 mmol, 75% yield) as a colourless solid after purification by reverse phase flash chromatography. HPLC: RT=1.01 min (Waters Acquity UPLC BEH C18 1.7 um 2.1×50 mm, CH₃CN/H₂O/0.05% TFA, 1 min. gradient, wavelength=254 nm); MS (ES): m/z=420 [M+1]⁺. ¹H NMR (499 MHz, DMSO-d₆) δ 12.82 (br s, 1H), 7.89 (d, J=7.5 Hz, 2H), 7.78 (d, J=8.6 Hz, 1H), 7.67 (d, J=7.4 Hz, 1H), 7.64 (d, J=7.4 Hz, 1H), 7.42 (td, J=7.4, 3.0 Hz, 2H), 7.34-7.27 (m, 2H), 7.16-7.11 (m, 1H), 7.08-6.97 (m, 2H), 4.26-4.12 (m, 5H), 3.15 (dd, J=13.8, 4.9 Hz, 1H), 2.83 (dd, J=13.8, 10.3 Hz, 1H), 2.20 (s, 3H).

### Preparation of (S)-2-((((9H-fluoren-9-yl)methoxy)carbonyl)amino)-3-(2-fluoro-5-methoxyphenyl)propanoic acid

Step 1. The compound was prepared following the same procedure of tert-butyl (S)-2-((((9H-fluoren-9-yl)methoxy)carbonyl)amino)-3-(2-fluoro-5-methoxyphenyl)propanoate (117.7 mg, 0.24 mmol, 50.4% yield) as a colourless solid after purification by flash chromatography. HPLC: RT=1.15 min (Waters Acquity UPLC BEH C18 1.7 um 2.1×50 mm, CH₃CN/H₂O/0.05% TFA, 1 min. gradient, wavelength=254 nm); MS (ES): m/z=436 [M-t-Bu]⁺. ¹H NMR (499 MHz, CHLOROFORM-d) δ 7.78 (d, J=7.5 Hz, 2H), 7.63-7.56 (m, 2H), 7.42 (t, J=7.4 Hz, 2H), 7.37-7.30 (m, 2H), 7.01-6.93 (m, 1H), 6.79-6.72 (m, 2H), 5.41 (br d, J=8.2 Hz, 1H), 4.62-4.55 (m, 1H), 4.41 (dd, J=10.4, 7.3 Hz, 1H), 4.31 (dd, J=10.5, 7.4 Hz, 1H), 4.26-4.20 (m, 1H), 3.75 (s, 3H), 3.17 (dd, J=13.9, 6.7 Hz, 1H), 3.11 (dd, J=14.4, 6.6 Hz, 1H), 1.45 (s, 9H).

Step 2. The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2-fluoro-5-methoxyphenyl)propanoic acid (79.5 mg, 0.183 mmol, 76% yield) as a colourless solid after purification by flash chromatography. HPLC: RT=0.98 min (Waters Acquity UPLC BEH C18 1.7 um 2.1×50 mm, CH₃CN/H₂O/0.05% TFA, 1 min. gradient, wavelength=254 nm); MS (ES): m/z=436 [M+1]⁺. Base peak of 214=fully deprotected amino acid fragment was also observed. ¹H NMR (499 MHz, DMSO-d₆) δ 12.84 (br s, 1H), 7.89 (d, J=7.5 Hz, 2H), 7.79 (d, J=8.6 Hz, 1H), 7.64 (t, J=8.4 Hz, 2H), 7.45-7.38 (m, 2H), 7.34-7.25 (m, 2H), 7.07 (t, J=9.2 Hz, 1H), 6.94 (dd, J=6.1, 3.2 Hz, 1H), 6.80 (dt, J=8.9, 3.6 Hz, 1H), 4.25-4.13 (m, 4H), 3.69 (s, 3H), 3.17 (dd, J=13.9, 4.6 Hz, 1H), 2.83 (dd, J=13.7, 10.7 Hz, 1H).

### Preparation of (S)-2-((((9H-fluoren-9-yl)methoxy)carbonyl)amino)-3-(2-methoxy-5-methylphenyl)propanoic acid

Step 1. The compound was prepared following the same procedure of tert-butyl (S)-2-((((9H-fluoren-9-yl)methoxy)carbonyl)amino)-3-(4-(trifluoromethoxy)phenyl)propanoate. The photoredox coupling afforded the desired product, tert-butyl (S)-2-((((9H-fluoren-9-yl)methoxy)carbonyl)amino)-3-(2-methoxy-5-methylphenyl)propanoate (73.9 mg, 0.15 mmol, 31.3% yield) as a colourless film after purification by flash chromatography. HPLC: RT=1.20 min (Waters Acquity UPLC BEH C18 1.7 um 2.1×50 mm, CH₃CN/H₂O/0.05% TFA, 1 min. gradient, wavelength=254 nm); MS (ES): m/z=488 [M-tBu+H]⁺. ¹H NMR (499 MHz, CHLOROFORM-d) δ 7.78 (d, J=7.6 Hz, 2H), 7.61-7.54 (m, 2H), 7.41 (t, J=7.4 Hz, 2H), 7.34-7.30 (m, 2H), 7.05 (dd, J=8.1, 1.5 Hz, 1H), 6.98 (d, J=1.4 Hz, 1H), 6.79 (d, J=8.3 Hz, 1H), 5.70 (br d, J=7.7 Hz, 1H), 4.49 (q, J=7.4 Hz, 1H), 4.33 (d, J=7.4 Hz, 2H), 4.25-4.18 (m, 1H), 3.82 (s, 3H), 3.10-3.02 (m, 2H), 2.26 (s, 3H), 1.43 (s, 9H).

Step 2. The final product was obtained following the same procedure of (S)-2-((((9H-fluoren-9-yl)methoxy)carbonyl)amino)-3-(4-(trifluoromethoxy)phenyl)propanoic acid. Removal of the tBu ester with HCl/dioxane afforded the desired (S)-2-((((9H-fluoren-9-yl)methoxy)carbonyl)amino)-3-(2-methoxy-5-methylphenyl)propanoic acid (44.7 mg, 0.104 mmol, 68.4% yield) as a colourless solid after purification by flash chromatography. HPLC: RT=1.02 min (Waters Acquity UPLC BEH C18 1.7 um 2.1×50 mm, CH₃CN/H₂O/0.05% TFA, 1 min. gradient, wavelength=254 nm); MS (ES): m/z=432 [M+H]⁺. ¹H NMR (499 MHz, DMSO-d₆) δ 12.61 (br s, 1H), 7.89 (d, J=7.5 Hz, 2H), 7.67 (d, J=7.5 Hz, 1H), 7.63 (d, J=7.5 Hz, 1H), 7.60 (br d, J=8.1 Hz, 1H), 7.42 (td, J=7.2, 3.5 Hz, 2H), 7.32 (td, J=7.5, 1.0 Hz, 1H), 7.30-7.26 (m, 1H), 7.02-6.97 (m, 2H), 6.84 (d, J=8.9 Hz, 1H), 4.26-4.10 (m, 4H), 3.75 (s, 3H), 3.12 (dd, J=13.5, 4.8 Hz, 1H), 2.72 (dd, J=13.4, 10.2 Hz, 1H), 2.16 (s, 3H).

### Preparation of (S)-2-((((9H-fluoren-9-yl)methoxy)carbonyl)amino)-3-hydroxy-3-methylbutanoic acid (99780-839-06)

Step 1. To a 10-L multi-neck round-bottomed flask was charged methyl (tert-butoxycarbonyl)-D-serinate (50 g, 228 mmol), diethyl ether (4200 mL). The mixture was cooled to −78° C. and methylmagnesium bromide (456 mL, 1368 mmol) was added dropwise over 30 min. The reaction was stirred at RT for 1 h. It was cooled to 0° C. and saturated NH₄Cl solution (1500 mL), was added dropwise and stirred for 10 min. The organic layer was separated and the aqueous layer was extracted with ethyl acetate (3×2000 mL). The combined organic layer was washed with brine, dried over Na₂SO₄, and concentrated at 40° C. to give a colorless thick liquid. The crude was purified by IPAC. Desired fractions were eluted at 50% EtOAc:petroleum ether mixture, and were collected and concentrated at 40° C. to give tert-butyl (R)-(1,3-dihydroxy-3-methylbutan-2-yl)carbamate (43.5 g, 87%) as a white solid. ¹H NMR (MeOD, 300 MHz) δ 3.70 (m, 1H), 3.48 (m, 1H), 3.21 (m, 1H), 1.35 (s, 9H), 1.13 (s, 3H), 1.05 (s, 3H).

Step 2. A 50-ml single neck round-bottomed flask was charged with tert-butyl (R)-(1,3-dihydroxy-3-methylbutan-2-yl)carbamate (43.0 g, 196 mmol), acetonitrile (650 mL) and was stirred till solution became clear. Sodium phosphate buffer (460 mL, 196 mmol) (pH=6.7, 0.67 M), (diacetoxyiodo)benzene (4.48 g, 13.92 mmol), and TEMPO (2.206 g, 14.12 mmol) were added sequentially and then the reaction was cooled to 0° C. and sodium chlorite (19.95 g, 221 mmol) was added. The color of the reaction turned black. The reaction was allowed to stir at 0° C. for 2 h. then at RT overnight. The orange colored reaction was quenched with saturated ammonium chloride solution (1000 mL) and a pH meter was used to adjust the pH=2 using 1.5 N HCl (330 mL). The aqueous solution was saturated with solid NaCl and extracted with ethyl acetate. The combined organic layer was washed with brine, dried over Na₂SO₄, and concentrated to obtain crude (S)-2-((tert-butoxycarbonyl)amino)-3-hydroxy-3-methylbutanoic acid (34.0 g, 74.3% yield) as an off-white solid and was taken directly to the next stage. ¹H NMR (MeOD, 300 MHz) δ 3.98 (s, 1H), 1.35 (s, 9H), 1.19 (s, 3H), 1.16 (9s, 3H).

Step 3. A 2000-mL single neck flask was charged with (S)-2-((tert-butoxycarbonyl)amino)-3-hydroxy-3-methylbutanoic acid (90 g, 386 mmol) in dioxane (450 mL) and was cooled to 0° C. 4N HCl in Dioxane (450 mL, 1800 mmol) was added dropwise over 10 min. The reaction was allowed to stir at RT for 3 h. It was concentrated and azetroped with toluene (2×) then stirred with ethyl acetate for 10 min. It was filtered and dried under vacuum to obtain crude (S)-2-amino-3-hydroxy-3-methylbutanoic acid, HCl (70 g, 107% yield) as a white solid and was taken directly to the next step.

Step 4. To a 3000-ml multi-neck round-bottomed flask was charged (S)-2-amino-3-hydroxy-3-methylbutanoic acid, HCl (70 g, 413 mmol), dioxane (1160 mL), and water (540 mL). The stirred solution became clear and a solution of sodium bicarbonate (104 g, 1238 mmol) in water (1160 mL) was added in one portion at RT. The reaction mass was allowed to stir at RT for 30 min. A solution of Fmoc-OSu (139 g, 413 mmol) in 1,4-dioxane (1460 mL) was added in one portion at RT. The reaction was allowed to stir at RT for 16 h. The reaction was concentrated to remove dioxane. To the resulting solution water was added and washed with ethyl acetate (3×1000 mL). The aqueous solution was acidified to pH 1-2 and extracted with ethyl acetate. The combined organic layer was washed with water, followed by brine, finally dried over Na₂SO₄, and concentrated to give an off-white solid (135.7 g). To remove the trapped dioxane and ethyl acetate the following procedure was followed: the solid was dissolved in ethyl acetate (1200 mL) and was stripped off with n-hexane (3000 mL). The slurry obtained was stirred for 10 min, filtered, and dried under vacuum to give (S)-2-((((9H-fluoren-9-yl)methoxy)carbonyl)amino)-3-hydroxy-3-methylbutanoic acid (112.0 g, 74.8 yield for two steps) as a white solid.

### Preparation of (S)-2-((((9H-fluoren-9-yl)methoxy)carbonyl)amino)-3-(3,4,5-trifluorophenyl)propanoic acid

Step 1. To a stirred solution of 2-((diphenylmethylene)amino)acetonitrile (100 g, 454 mmol) in DCM (1000 mL), 5-(bromomethyl)-1,2,3-trifluorobenzene (66.5 mL, 499 mmol) and benzyltrimethylammonium chloride (16.86 g, 91 mmol) was added. To this, 10 M NaOH (136 mL, 1362 mmol) solution was added and stirred at RT overnight. After 26 h, the reaction mixture was diluted with water (500 mL) and the DCM layer was separated. The aqueous layer was further extracted with DCM (2×250 mL). The organic layer was combined, washed with water and brine solution, dried over Na₂SO₄, filtered, and concentrated under vacuum. The crude compound was purified by flash column chromatography (1.5 kg, silica gel, 0-10% ethylacetate/petroleum ether mixture) and the desired fractions were collected and concentrated to afford 2-((diphenylmethylene)amino)-3-(3,4,5-trifluorophenyl)propanenitrile (140 g, 384 mmol, 85% yield) as a yellow solid. Analysis condition E: Retention time=3.78 min; ESI-MS(+) m/z [M+H]⁺: 365.2.

Step 2. To a stirred solution of 2-((diphenylmethylene)amino)-3-(3,4,5-trifluorophenyl)propanenitrile (80 g, 220 mmol) in 1,4-dioxane (240 mL), was added conc. HCl (270 mL, 3293 mmol) and the mixture was stirred at 90° C. for 16 h. The reaction mixture was taken as such for the next step.

Step 3. To the crude aqueous dioxane solution from the previous was added 10 N NaOH solution until the solution was neutral. Na₂CO₃ (438 ml, 438 mmol) was then added, followed by the addition of Fmoc-OSu (81 g, 241 mmol). The mixture was stirred at RT overnight. The aqueous solution was acidified with 1.5 N HCl till pH=2 and the solid formed was filtered, and dried to afford the crude compound. It was slurried initially with 5% EtOAc/petroleum ether for 30 min and filtered. The filtered compound was further slurried with ethyl acetate for 20 min and filtered to get the crude racemic 2-((((9H-fluoren-9-yl)methoxy)carbonyl)amino)-3-(3,4,5-trifluorophenyl)propanoic acid (90 g, 204 mmol, 93% yield) as an off-white solid. This racemic compound was separated into two isomers by SFC purification to provide the desired isomers. After concentration of the desired isomer, it was slurried with 5% EtOAc/petroleum ether and filtered to get (S)-2-((((9H-fluoren-9-yl)methoxy)carbonyl)amino)-3-(3,4,5-trifluorophenyl)propanoic acid (43 g, 95 mmol, 43.3% yield) as an off-white solid. ¹H NMR (MeOD, 400 MHz) δ 7.78 (d, J=7.2 Hz, 2H), 7.60 (t, J=8.0 Hz, 2H), 7.38 (t, J=8.0 Hz, 2H), 7.28 (t, J=7.6 Hz, 2H), 7.01 (t, J=7.8 Hz, 2H), 4.48-4.26 (m, 3H), 4.18 (m, 1H), 3.18 (m, 1H), 2.91 (m, 1H). ¹⁹F (MeOD, 376 MHz) δ −137.56 (d, J=19.6 Hz, 2F), −166.67 (t, J=19.6 Hz, 1F). Analysis condition E: Retention time=3.15 min; ESI-MS(+) m/z [M+H]⁺: 442.2.

The other fraction was concentrated to provide (R)-2-((((9H-fluoren-9-yl)methoxy)carbonyl)amino)-3-(3,4,5-trifluorophenyl)propanoic acid (40 g, 91 mmol, 41.4% yield) as an off-white solid.

### Preparation of (S)-2-((((9H-fluoren-9-yl)methoxy)carbonyl)amino)-4-(tert-butoxy)-3,3-dimethyl-4-oxobutanoic acid

Step 1. To a stirred solution of 4-(tert-butyl) 1-methyl L-aspartate, HCl salt (34 g, 142 mmol) in acetonitrile (550 mL), was added lead(II) nitrate (47.0 g, 142 mmol), potassium phosphate (66.2 g, 312 mmol), and TEA (19.77 mL, 142 mmol) under nitrogen atmosphere. The mixture was cooled to 0° C. then a solution of 9-bromo-9-phenylfluorene (43.3 g, 135 mmol) in acetonitrile (100 mL) was added. The reaction mixture was stirred at RT for 48 h and the reaction progress was monitored by TLC (50% EA in PE) and LCMS. The reaction mixture was filtered over celite, washed with chloroform, and evaporated to provide a thick pale yellow liquid, to which ethyl acetate (3500 mL) was added. The EtOAc layer was washed with 5% citric acid solution (500 mL) followed by brine solution. The organic layer was dried over sodium sulfate and evaporated under reduced pressure to provide a pale yellow thick liquid, which was scratched with petroleum ether and filtered to obtain 4-(tert-butyl) 1-methyl (9-phenyl-9H-fluoren-9-yl)-L-aspartate (55 g, 124 mmol, 87% yield) as a white solid. Analysis condition L: Retention time=1.73 min; ESI-MS(+) m/z [M+Na]*: 466.40.

Step 2. A solution of 4-(tert-butyl) 1-methyl (9-phenyl-9H-fluoren-9-yl)-L-aspartate (22.5 g, 50.7 mmol) was cooled to −78° C. under Ar and a solution of KHMDS (127 mL, 127 mmol, 1 M in THF) was added over 30 min while stirring. The reaction was allowed to warm to −40° C., and methyl iodide (9.52 mL, 152 mmol) was added dropwise. The reaction was stirred at −40° C. for 5 h. The reaction was monitored by TLC and LCMS. Saturated NH₄Cl (400 mL) was added followed by H₂O (100 mL). The resulting mixture was extracted with EtOAc (3×) and the combined organic extracts were washed with 2% citric acid (200 mL), aq. NaHCO₃ (200 mL), and brine. The organic layer was dried over anhydrous Na₂SO₄, evaporated in vacuo, and recrystallized from hexanes to give 1-(tert-butyl) 4-methyl (S)-2,2-dimethyl-3-((9-phenyl-9H-fluoren-9-yl)amino)succinate (18.5 g, 39.2 mmol, 77% yield) as a white solid, which was taken for the next step. Analysis condition L: Retention time=2.04 min; ESI-MS(+) m/z [M+Na]⁺: 494.34.

Step 3. A stirred solution of 1-(tert-butyl) 4-methyl (S)-2,2-dimethyl-3-((9-phenyl-9H-fluoren-9-yl)amino)succinate (24 g, 50.9 mmol) in methanol (270 mL) and ethyl acetate (100 mL) was degassed with nitrogen. Pd—C(2.71 g, 2.54 mmol) (10% by weight) was added, and the mixture was flushed with hydrogen gas and then stirred at RT in a 1-liter capacity autoclave at 50 psi overnight. The reaction mixture was filtered through a celite pad, and washed with a mixture of methanol and ethyl acetate. The combined solvents were evaporated to dryness and the precipitated white solid was removed by filtration to obtain a pale yellow liquid 1-(tert-butyl) 4-methyl (S)-3-amino-2,2-dimethylsuccinate (11.7 g) which was taken as such for the next step.

Step 4. To a stirred solution of 1-(tert-butyl) 4-methyl (S)-3-amino-2,2-dimethylsuccinate (11.0 g, 47.6 mmol), cooled in an ice bath, was added lithium hydroxide (428 mL, 86 mmol, 0.2 M solution in water) and the reaction was slowly brought to RT. The reaction was monitored by TLC and LCMS. The reaction mixture was evaporated and directly taken to the next step. To a stirred solution of (S)-2-amino-4-(tert-butoxy)-3,3-dimethyl-4-oxobutanoic acid (15 g, 69.0 mmol) (which was in water from the previous batch) in acetonitrile (200 mL) cooled to 0° C., was added sodium bicarbonate (5.80 g, 69.0 mmol) and Fmoc-OSu (46.6 g, 138 mmol). The reaction mixture was stirred at RT overnight. It was acidified with 2 N HCl to pH=4, then extracted with ethyl acetate (3×500 mL), and the combined organic layer was washed with brine, dried over sodium sulfate, and evaporated to get an off-white solid, which was purified by ISCO flash chromatography with 20% EA in petroleum ether to get (S)-2-((((9H-fluoren-9-yl)methoxy)carbonyl)amino)-4-(tert-butoxy)-3,3-dimethyl-4-oxobutanoic acid (12.2 g, 26.9 mmol, 39.0% yield) as a white solid. ¹HNMR (CDCl₃, 400 MHz) δ 7.77 (d, J=7.6 Hz, 2H), 7.60 (m, 2H), 7.42 (t, J=8.0 Hz, 2H), 7.33 (t, J=7.6 Hz, 2H), 4.65 (m, 2H), 4.34 (m, 1H), 4.25 (m, 1H), 3.18 (m, 1H), 1.40-1.27 (m, 6H). Analysis condition E: Retention time=1.90 min; ESI-MS(+) m/z [M+H]⁺: 440.2.

### Preparation of (S)-2-((((9H-fluoren-9-yl)methoxy)carbonyl)amino)-3-(3-(tert-butoxycarbonyl)phenyl)propanoic acid

Step 1. To a solution of (S)-2-(1,3-dioxoisoindolin-2-yl)propanoic acid (80 g, 365 mmol), O-methylhydroxylamine hydrochloride (36.6 g, 438 mmol) in CH₂Cl₂ (2000 mL), was added TEA (153 mL, 1095 mmol) at RT. The reaction was cooled to 0° C., 1-propanephosphonic anhydride (326 mL, 547 mmol) was added dropwise. The reaction was stirred at RT for 2 h. It was quenched with saturated ammonium chloride (500 mL) and extracted with EtOAc (3×300 mL). The combined organic layers were washed with saturated brine, dried over Na₂SO₄, and concentrated under reduced pressure. The crude product was purified via combiflash using a 120 g silica column with 38 to 45% EtOAc in petroleum ether to give (S)-2-(1,3-dioxoisoindolin-2-yl)-N-methoxypropanamide (80 g, 322 mmol, 88% yield). ¹H NMR (DMSO-d₆, 400 MHz) δ 11.36 (s, 1H), 7.91-7.85 (m, 4H), 4.75-4.69 (m, 1H), 3.56 (s, 3H), 1.51 (d, J=7.6 Hz, 3H).

Step 2. To a solution of (S)-2-(1,3-dioxoisoindolin-2-yl)-N-methoxypropanamide (20 g, 81 mmol), palladium(II) acetate (1.809 g, 8.06 mmol), silver acetate (26.9 g, 161 mmol) placed in a 1000-ml seal tube, was added tert-butyl 3-iodobenzoate (36.8 g, 121 mmol), 2,6-Lutidine (2.395 ml, 24.17 mmol), and HIP (300 ml) at 25° C. under N₂ atmosphere. The reaction was stirred for 15 min at 25° C. under N₂ and then heated up to 80° C. for 24 h with vigorous stirring. The reaction mixture was filtered through celite and washed with DCM (200 mL). The combined organic layer was concentrated under reduced pressure. The crude product was purified via combiflash using a 220 g silica column eluting with 25 to 30% EtOAc:CHCl₃ to obtain the desired product tert-butyl (S)-3-(2-(1,3-dioxoisoindolin-2-yl)-3-(methoxyamino)-3-oxopropyl)benzoate (11 g, 25.9 mmol, 32.2% yield). Analysis condition E: Retention time=2.52 min; ESI-MS(+) m/z [M−H]⁺: 423.2. ¹H NMR (DMSO-d₆, 400 MHz) δ 11.46 (s, 1H), 7.82 (m, 4H), 7.63 (d, J=7.6 Hz, 1H), 7.54 (s, 1H), 7.40 (d, J=7.6 Hz, 1H), 7.30 (t, J=7.6 Hz, 1H), 4.93-4.89 (m, 1H), 3.59 (s, 3H), 3.56-3.49 (m, 1H), 3.36-3.27 (m, 1H), 1.40 (s, 9H).

Step 3. To a solution of tert-butyl (S)-3-(2-(1,3-dioxoisoindolin-2-yl)-3-(methoxyamino)-3-oxopropyl)benzoate (15 g, 35.3 mmol) in methanol (200 mL), (diacetoxyiodo)benzene (12.52 g, 38.9 mmol) was added at RT. The temperature was slowly raised to 80° C. and stirred for 3 h at 80° C. The reaction was concentrated under reduced pressure to get the crude product. It was purified with silica gel chromatography (100-200 mesh eluting with 20% EA:hexane) to obtain the desired compound tert-butyl (S)-3-(2-(1,3-dioxoisoindolin-2-yl)-3-methoxy-3-oxopropyl)benzoate (10 g, 24.42 mmol, 69.1% yield. ¹H NMR (CDCl₃, 400 MHz) δ 7.80-7.76 (m, 4H), 7.72-7.68 (m, 2H), 7.34-7.26 (m, 1H), 7.25-7.23 (m, 1H), 5.14 (dd, J=10.8, 5.6 Hz, 1H), 3.76 (s, 3H), 3.65-3.49 (m, 2H), 1.50 (s, 9H).

Step 4. To a solution of tert-butyl (S)-3-(2-(1,3-dioxoisoindolin-2-yl)-3-methoxy-3-oxopropyl)benzoate (15 g, 36.6 mmol) in methanol (25 mL) ethylenediamine (12.25 mL, 183 mmol) was added at RT. The reaction temperature was slowly raised to 40° C. and stirred for 3 h at 40° C. The mixture was concentrated under reduced pressure to get the crude product. It was purified with silica gel chromatography (100-200 mesh eluting with 20% EA:hexane) to obtain the desired compound tert-butyl (S)-3-(2-amino-3-methoxy-3-oxopropyl)benzoate (8.3 g, 29.7 mmol, 81% yield). ¹H NMR (DMSO-d₆, 400 MHz) δ 8.32 (s, 1H), 7.77-7.72 (m, 2H), 7.46-7.38 (m, 1H), 3.61-3.57 (m, 4H), 2.96-2.91 (m, 1H), 2.85-2.82 (m, 1H), 1.79 (br. s, 2H), 1.55 (s, 9H).

Step 5. To a solution of tert-butyl (S)-3-(2-amino-3-methoxy-3-oxopropyl)benzoate (10 g, 35.8 mmol) in dioxane (150 mL), sodium bicarbonate (6.01 g, 71.6 mmol) was added followed by the addition of 9-fluorenylmethyl chloroformate (13.89 g, 53.7 mmol) at RT. The reaction was stirred for 12 h at RT. It was diluted with water and extracted with ethyl acetyate. The organic layer was concentrated under reduced pressure to get the crude product. It was purified via silica gel chromatography (100-200 mesh eluting with 20% EA: hexane) to obtain the desired compound tert-butyl (S)-3-(2-((((9H-fluoren-9-yl)methoxy)carbonyl)amino)-3-methoxy-3-oxopropyl)benzoate (15 g, 29.9 mmol, 84% yield).

Step 6. To a solution of tert-butyl (S)-3-(2-((((9H-fluoren-9-yl)methoxy)carbonyl)amino)-3-methoxy-3-oxopropyl)benzoate (18.00 g, 35.9 mmol) in THE (150 mL) and H₂O (150 mL) at RT, lithium hydroxide monohydrate (1.66 g, 39.5 mmol) was added. The reaction was stirred for 2 h at RT. The reaction was concentrated under reduced pressure to remove THF. In the basic medium, the mixture was extracted with diethyl ether to remove the non polar impurities. The aqueous layer was acidified with aqueous citric acid solution and extracted with ethyl acetate. The organic layer was dried over sodium sulphate and concentrated under reduced pressure to get the desired compound as a gummy solid which was further lyopholized to give (S)-2-((((9H-fluoren-9-yl)methoxy)carbonyl)amino)-3-(3-(tert-butoxycarbonyl)phenyl)propanoic acid (16 g, 32.72 mmol, quantative yield) as an off-white solid. ¹H NMR (CDCl₃, 400 MHz) δ 7.86 (t, J=7.6 Hz, 2H), 7.75 (d, J=7.6 Hz, 1H), 7.66-7.59 (m, 2H), 7.52 (m, 2H), 7.41-7.37 (m, 3H), 7.31-7.24 (m, 2H), 4.21-4.16 (m, 4H), 3.17 (m, 1H), 2.96 (m, 1H), 1.53 (br, s. 9H). Analysis condition E: Retention time=3.865 min; ESI-MS(+) m/z [M−H]⁺: 486.2.

### Preparation of (S)-2-((((9H-fluoren-9-yl)methoxy)carbonyl)amino)-3-(m-tolyl)propanoic acid

Compound was synthesized following the similar procedures of (S)-2-((((9H-fluoren-9-yl)methoxy)carbonyl)amino)-3-(3-(tert-butoxycarbonyl)phenyl)propanoic acid. Analysis condition E: Retention time=3.147 min; ESI-MS(+) m/z [M+H]⁺: 402.0. ¹H NMR (DMSO-d₆, 300 MHz) δ 7.88 (d, J=7.5 Hz, 2H), 7.64 (t, J=6.8 Hz, 2H), 7.44 (t, J=7.5 Hz, 2H), 7.36-7.28 (m, 2H), 7.18 (t, J=7.5 Hz, 1H), 7.09-7.02 (m, 3H), 4.24-4.17 (m, 4H), 3.21-3.04 (m, 1H), 2.89-2.81 (m, 1H), 2.26 (s, 3H) ppm.

### Preparation ethyl (S)-5-((tert-butoxycarbonyl)amino)-2-(((S)-mesitylsulfinyl)amino)-3,3-dimethylpentanoate

Step 1: To a 1000-ml flask equipped with a septum inlet and magnetic stirring bar was added bismuth(III) chloride (5.25 g, 16.64 mmol). The flask was connected to an argon line and thionyl chloride (501 mL, 6864 mmol) were added by syringe. To the suspension was added mesitylene (100 g, 832 mmol). The flask was equipped with a condenser, connected to an oil bubbler and the reaction mixture was heated in an oil bath at 60° C. for 5 h. During this time the color of the solution became red-orange and HCl evolved from the solution. The reaction was monitored by LCMS. The flask was cooled in an ice bath and the excess of thionyl chloride was removed under reduced pressure yielding to an orange liquid. In order to remove the catalyst, 2000 mL of pentane was added, stirred and filtered through celite, and the bed was washed with pentane (2×500 mL). The organic phase was collected and evaporated under reduced pressure to give 2,4,6-trimethylbenzenesulfinic chloride (151 g, 745 mmol, 90% yield) as a pale yellow solid. The compound was taken to the next step without further purification. ¹H NMR (400 MHz, CDCl₃) δ 7.07-6.76 (m, 2H), 2.66 (s, 6H), 2.38-2.24 (m, 3H) ppm.

Step 2. A stirred solution of 2,4,6-trimethylbenzenesulfinic chloride (155 g, 765 mmol) was prepared in diethyl ether (1500 mL) an cooled to −40° C. In a separate setup, (2 L multi neck RBF) diethyl ether (900 mL) was added, and then ammonia gas was bubbled 30 minutes at −40° C. Next, this purged solution was added to the above reaction mass at −40° C. After it had warmed to RT the reaction mixture was stirred for 2 hours and monitored by open access LCMS until starting material was absent. The reaction was then stirred at room temperature overnight according to the given procedure. The reaction was monitored by TLC and open access LCMS, TLC wise starting material was absent. Workup: The reaction mixture was diluted with ethyl acetate (3000 mL) and washed with water (2000 ml). The organic layer was separated and the aqueous phase was again extracted with ethyl acetate (1×500 mL). The combined organic layer was washed with brine (1×800 mL). The combined organic layer was dried (Na₂SO₄), filtered, and concentrated under reduced pressure to obtain (235 g) as a pale brown solid. The product (235 g) was recrystallized from 10% ethyl acetate/petroleum ether (500 mL), stirred, filtered, and dried to afford mesitylenesulphinamide (125 g) racemate as a white solid. The compound was submitted for the SFC method development. Two peaks were collected from SFC. The solvent was concentrated to give Peak-1 (Undesired): (R)-2,4,6-trimethylbenzenesulfinamide (51.6 g, 265 mmol, 34.6% yield) as a white colour solid. ¹H NMR (400 MHz, DMSO-d₆) δ 7.01-6.68 (m, 2H), 6.23-5.77 (m, 2H), 2.52-2.50 (m, 6H), 2.32-1.93 (m, 3H) and Peak-2 (desired): (S)-2,4,6-trimethylbenzenesulfinamide (51.6 g, 267 mmol, 35.0% yield) as a white colored solid. ¹H NMR (400 MHz, DMSO-d₆) δ 6.87 (s, 2H), 6.16-5.82 (m, 2H), 2.53-2.50 (m, 6H), 2.34-1.93 (m, 3H).

Step 3. To a well stirred solution of (S)-2,4,6-trimethylbenzenesulfinamide (15.5 g, 85 mmol) in dichloromethane (235 mL) and 4A molecular sieves (84.5 g), was added ethyl 2-oxoacetate in toluene (25.9 mL, 127 mmol) and pyrrolidine (0.699 mL, 8.46 mmol). The reaction mixture was stirred at room temperature for overnight. The reaction was repeated and the two batches were combined together for work up. The reaction was filtered through celite and the bed was washed with DCM. The solvents were removed under reduced pressure to obtain the crude (55 g) as a brownish color mass. The crude compound was purified by ISCO (Column size: 300 g silica column. Adsorbent: 60-120 silica mesh, Mobile phase:40% EtOAc/Pet ether) and the product was collected at 15-20% of EtOAc. The fractions were concentrated to obtain ethyl (S,E)-2-((mesitylsulfinyl)imino)acetate (16.5 g, 57.4 mmol, 67.9% yield) as a colorless liquid. The compound slowly solidified as an off white solid. ¹H NMR (400 MHz, CDCl₃) δ=8.27 (s, 1H), 7.04-6.70 (m, 2H), 4.59-4.21 (m, 2H), 2.55-2.44 (m, 6H), 2.36-2.23 (m, 3H), 1.51-1.30 (m, 3H). 2.670 min. 268.2 (M+H).

Step 4. TCNHPI esters were prepared according to the previously reported general procedure (ACIE paper and references therein): A round-bottom flask or culture tube was charged with carboxylic acid (1.0 equiv), N-hydroxytetrachlorophthalimide (1.0-1.1 equiv) and DMAP (0.1 equiv). Dichloromethane was added (0.1-0.2 M), and the mixture was stirred vigorously. Carboxylic acid (1.0 equiv) was added. DIC (1.1 equiv) was then added dropwise via syringe, and the mixture was allowed to stir until the acid was consumed (determined by TLC). Typical reaction times were between 0.5 h and 12 h. The mixture was filtered (through a thin pad of Celite®, SiO₂, or frit funnel) and washed with additional CH₂Cl₂/Et₂₀. The solvent was removed under reduced pressure, and purification of the crude mixture by column chromatography afforded the desired TCNHPI redox-active ester. If necessary, the TCNHPI redox-active ester could be further recrystallized from CH₂Cl₂/MeOH.

Step 5. 4,5,6,7-tetrachloro-1,3-dioxoisoindolin-2-yl-4-((tert-butoxycarbonyl)amino)-2,2-dimethylbutanoate was obtained as a white solid following General Procedure for the synthesis of TCNHPI redox-active esters on 5.00 mmol scale. Purification by column (silica gel, gradient from CH₂Cl₂ to 10:1 CH₂Cl₂:Et₂O) afforded 2.15 g (84%) of the title compound. ¹H NMR (400 MHz, CDCl₃): δ 4.89 (br s, 1H), 3.30 (q, J=7.0 Hz, 2H), 1.98 (t, J=7.6 Hz, 2H), 1.42 (s, 15H) ppm. ¹³C NMR (151 MHz, CDCl₃): δ 173.1, 157.7, 156.0, 141.1, 130.5, 124.8, 79.3, 40.8, 40.2, 36.8, 28.5, 25.2 ppm. HRMS (ESI-TOF): calc'd for C₁₉H₂OCl₄N₂NaO₆ [M+Na]⁺: 534.9968, found: 534.9973.

Step 6. Ethyl (S)-5-((tert-butoxycarbonyl)amino)-2-(((S)-mesitylsulfinyl)amino)-3,3-dimethylpentanoate was made using the General procedures for decarboxylative Amino acid synthesis in reference ACIE. A culture tube was charged with TCNHPI redox-active ester A (1.0 mmol), sulfinimine B (2.0 mmol), Ni(OAc)₂·4H₂O (0.25 mmol, 25 mol %), and Zinc (3 mmol, 3 equiv). The tube was then evacuated and backfilled with argon (three times). Anhydrous NMP (5.0 mL, 0.2 M) was added using a syringe. The mixture was stirred overnight at RT. Then, the reaction mixture was diluted with EtOAc, washed with water, brine and dried over MgSO₄. Upon filtration, the organic layer was concentrated under reduced pressure (water bath at 30° C.), and purified by flash column chromatography (silica gel) to provide the product. Purification by column (2:1 hexanes:EtOAc) afforded 327.6 mg (72%) of the title compound ethyl (S)-5-((tert-butoxycarbonyl)amino)-2-(((S)-mesitylsulfinyl)amino)-3,3-dimethylpentanoate as a colorless oil. ¹H NMR (600 MHz, CDCl₃): δ 6.86 (s, 2H), 5.04 (d, J=10.1 Hz, 1H), 4.47 (s, 1H), 4.28-4.16 (m, 2H), 3.66 (d, J=10.1 Hz, 1H), 3.27-3.05 (m, 2H), 2.56 (s, 6H), 2.28 (s, 3H), 1.54-1.46 (m, 2H), 1.43 (s, 9H), 1.30 (t, J=7.2 Hz, 3H), 0.96 (s, 6H) ppm. ¹³C NMR (151 MHz, CDCl₃): δ 172.5, 155.9, 141.1, 137.9, 136.9, 131.0, 79.4, 65.5, 61.7, 38.8, 37.1, 36.5, 28.5, 23.9, 23.6, 21.2, 19.4, 14.3 ppm. HRMS (ESI-TOF): calc'd for C₂₃H₃₉N₂O₅S [M+H]⁺: 455.2574, found: 455.2569.

Step 7. 2-((((9H-fluoren-9-yl)methoxy)carbonyl)amino)-5-((tert-butoxycarbonyl)amino)-3,3-dimethylpentanoic acid: A culture tube was charged with ethyl (S)-5-((tert-butoxycarbonyl)amino)-2-(((S)-mesitylsulfinyl)amino)-3,3-dimethylpentanoate (0.5 mmol, 1.0 equiv). HCl (4.0 equiv) in MeOH (0.3 M) was added via syringe and the resulting mixture was stirred at RT for ca. 10 min (screened by TLC). After the reaction, Et₃N was added until pH=7 and the solvents were removed under reduced pressure. LiOH (2 equiv) in MeOH/H₂O (2:1, 0.04 M) was added to the crude mixture. The reaction was stirred at 60° C. overnight. On completion, HCl in MeOH (0.3 M) was added until pH=7 and the solvents were removed under reduced pressure. The crude mixture was dissolved in 9% aqueous Na₂CO₃ (5 mL) and dioxane (2 mL). It was slowly added at 0° C. to a solution of Fmoc-OSu (1.2 equiv) in dioxane (8 mL). The mixture was stirred at 0° C. for 1 h and then allowed to warm to RT. After 10 h, the reaction mixture was quenched with HCl (0.5 M), reaching pH 3, and then diluted with EtOAc. The aqueous phase was extracted with EtOAc (3×15 mL), and the combined organic layers were washed with brine, dried over Na₂SO₄, filtered, and the solvent was removed under reduced pressure. The crude mixture was then purified by flash column chromatography (silica gel, 2:1 hexanes EtOAc) to afford the product ethyl (S)-5-((tert-butoxycarbonyl)amino)-2-(((S)-mesitylsulfinyl)amino)-3,3-dimethylpentanoate in 68% overall yield and 95% ee as a colorless oil. ¹H NMR (600 MHz, CDCl₃): δ 7.76 (d, J=7.5 Hz, 2H), 7.63-7.54 (m, 2H), 7.39 (td, J=7.3, 2.6 Hz, 2H), 7.33-7.28 (m, 2H), 5.50 (br s, 1H), 4.68 (br s, 1H), 4.45-4.43 (m, 1H), 4.38-4.35 (m, 1H), 4.30 (d, J=7.9 Hz, 1H), 4.21 (t, J=6.8 Hz, 1H), 3.27 (br s, 1H), 3.16 (br s, 1H), 1.63-1.50 (m, 2H), 1.43 (s, 9H), 1.09-0.76 (m, 6H) ppm. ¹³C NMR (151 MHz, CDCl₃): δ 185.8, 174.3, 156.5, 144.0, 143.9, 141.5, 127.9, 127.2, 125.24, 125.21, 120.2, 120.1, 79.8, 67.2, 60.9, 47.4, 39.2, 36.8, 29.9, 28.6, 23.9 ppm. HRMS (ESI-TOF): calc'd for C₂₇H₃₅N₂O₆ [M+H]⁺: 483.2490, found: 483.2489.

### Preparation of (S)-2-((((9H-fluoren-9-yl)methoxy)carbonyl)amino)-3-(4,4-difluorocyclohexyl)propanoic acid

Final product was obtained following similar procedures of ethyl (S)-5-((tert-butoxycarbonyl)amino)-2-(((S)-mesitylsulfinyl)amino)-3,3-dimethylpentanoate. The synthesis afforded the desired (S)-2-((((9H-fluoren-9-yl)methoxy)carbonyl)amino)-3-(4,4-difluorocyclohexyl)propanoic acid (60 mg, 0.14 mmol, 27.9% yield) as a white solid after purification by reverse phase HPLC. ¹H NMR (500 MHz, CDCl₃) δ 7.79 (br d, J=7.5 Hz, 2H), 7.61 (br s, 2H), 7.43 (s, 2H), 7.36-7.31 (m, 2H), 5.24-5.06 (m, 1H), 4.57-4.36 (m, 3H), 4.29-4.16 (m, 1H), 2.19-1.99 (m, 2H), 1.97-1.18 (m, 9H).

### Preparation of (2S)-5-(tert-butoxy)-2-({[(9H-fluoren-9-yl)methoxy]carbonyl}amino)-3,3-dimethyl-5-oxopentanoic acid

Step 1 A solution of 4,4-dimethyldihydro-2H-pyran-2,6(3H)-dione (8.29 g, 58.3 mmol) in dry toluene (100 mL) was slowly added to a solution of (R)-2-amino-2-phenylethan-1-ol (10 g, 72.9 mmol) in dry toluene (100 mL) and CH₂Cl₂ (20 mL) at room temperature. The reaction mixture was then heated to 60° C. and reacted for 12 h. It was cooled to room temperature until a white solid was formed. The solid was filtered and washed with 1:1 EtOAc/CH₂Cl₂ to afford the crude desired compound (R)-5-((2-hydroxy-1-phenylethyl)amino)-3,3-dimethyl-5-oxopentanoic acid (11.9 g, 41.0 mmol, 56.2% yield) without further purification. ¹H NMR (300 MHz, DMSO-d₆) δ 8.41 (br d, J=7.9 Hz, 1H), 7.44-7.32 (m, 2H), 7.32-7.27 (m, 4H), 7.26-7.18 (m, 1H), 4.89-4.80 (m, 1H), 4.14-3.98 (m, 1H), 3.63-3.43 (m, 3H), 2.27-2.18 (m, 4H), 2.08 (s, 1H), 1.99 (s, 1H), 1.17 (t, J=7.2 Hz, 1H), 1.00 (d, J=4.5 Hz, 6H), 0.92 (s, 1H).

Step 2 (R)-5-((2-Hydroxy-1-phenylethyl)amino)-3,3-dimethyl-5-oxopentanoic acid (12 g, 43.0 mmol) was dissolved in a solution of benzyltrimethylammonium chloride (8.93 g, 48.1 mmol) in DMA (250 mL). K₂CO₃ (154 g, 1117 mmol) was added to the above solution followed by the addition of 2-bromo-2-methylpropane (235 mL, 2091 mmol). The reaction mixture was stirred at 55° C. for 24 h. The reaction mixture was then diluted with EtOAc (100 mL), washed with H₂O (50 mL×3), and brine (50 mL). The organic phase was dried over Na₂SO₄, concentrated under vacuo, and purified by flash column chromatography on silica gel (CH₂Cl₂/MeOH, 15:1) to give tert-butyl (R)-5-((2-hydroxy-1-phenylethyl)amino)-3,3-dimethyl-5-oxopentanoate (6.0 g, 17.89 mmol, 41.6% yield). Analytical LC/MS Condition M: 1.96 min, 336.3 [M+H]⁺. ¹H NMR (300 MHz, DMSO-d₆) d=8.14 (br d, J=8.3 Hz, 1H), 7.33-7.25 (m, 4H), 7.25-7.17 (m, 1H), 4.90-4.77 (m, 2H), 3.52 (br t, J=5.7 Hz, 2H), 3.34 (s, 1H), 2.94 (s, 1H), 2.78 (s, 1H), 2.20 (d, J=14.0 Hz, 4H), 1.97 (d, J=9.8 Hz, 2H), 1.41-1.31 (m, 9H), 1.00 (d, J=1.1 Hz, 6H).

Step 3 tert-Butyl (R)-5-((2-hydroxy-1-phenylethyl)amino)-3,3-dimethyl-5-oxopentanoate (6 g, 17.89 mmol) and 2,3-dichloro-5,6-dicyano-p-benzoquinone (6.09 g, 26.8 mmol) was dissolved in dry dichloromethane (70 mL) under Ar. Triphenylphosphine (7.04 g, 26.8 mmol) was added to the above solution. The reaction mixture was stirred at room temperature for 2 h. The crude product was then concentrated under vacuo and purified by flash column chromatography on silica gel (EtOAc/Hexanes, 1:5) to give tert-butyl (R)-3,3-dimethyl-4-(4-phenyl-4,5-dihydrooxazol-2-yl)butanoate (5.6 g, 17.64 mmol, 99% yield). ESI-MS(+) m/z: 318.3 [M+H]⁺. ¹H NMR (300 MHz, DMSO-d₆) d=7.41-7.18 (m, 5H), 5.18 (t, J=9.1 Hz, 1H), 4.59 (dd, J=8.7, 10.2 Hz, 1H), 3.94-3.85 (m, 1H), 3.94-3.85 (m, 1H), 3.95-3.84 (m, 1H), 4.10-3.84 (m, 1H), 2.43-2.22 (m, 4H), 1.40 (s, 9H), 1.09 (d, J=1.9 Hz, 6H).

Step 4 A solution of tert-butyl (R)-3,3-dimethyl-4-(4-phenyl-4,5-dihydrooxazol-2-yl)butanoate (5.6 g, 17.64 mmol) in EtOAc (250 mL) was added selenium dioxide (4.89 g, 44.1 mmol) and refluxed for 2 h. The reaction mixture was then cooled to room temperature and stirred for 12 h. The crude product was then concentrated in vacuo and purified by flash column chromatography on silica gel (EtOAc/Hexanes, 1:7) to afford tert-butyl (R)-3-methyl-3-(2-oxo-5-phenyl-5,6-dihydro-2H-1,4-oxazin-3-yl)butanoate (1.3 g, 3.92 mmol, 22.23% yield) as a colorless liquid. ESI-MS(+) m/z: 332.2 [M+H]⁺. ¹H NMR (CDCl₃) δ 1.37 (s, 3H), 1.42 (s, 9H), 1.44 (s, 3H), 2.59 (d, J=15.5 Hz, 1H), 3.12 (d, J=15.5 Hz, 1H), 4.32 (t, J=11.1 Hz, 1H), 4.47 (dd, J=4.3 Hz, J=6.7 Hz, 1H), 4.80 (dd, J=4.3 Hz, J=6.7 Hz, 1H), 7.35-7.39 (m, 5H). ¹³C NMR (CD3Cl) δ 26.40, 27.29, 28.00, 40.84, 45.94, 59.72, 70.88, 80.63, 127.13, 127.92, 128.65, 137.58, 155.07, 167.46, 171.95.

Step 5 Platinum(IV) oxide monohydrate (130 mg, 0.530 mmol) was added to a solution of tert-butyl (R)-3-methyl-3-(2-oxo-5-phenyl-5,6-dihydro-2H-1,4-oxazin-3-yl)butanoate (1.3 g, 3.92 mmol) in methanol (50 mL). The reaction flask was purged with H₂ (3×) and stirred under H₂ for 24 h. After venting the vessel, the reaction mixture was filtered through Celite, and the filtrate was washed with EtOAc. The crude product was concentrated under vacuo and purified by flash column chromatography on silica gel (EtOAc/Hexanes, 1:8) to give tert-butyl 3-methyl-3-((3S,5R)-2-oxo-5-phenylmorpholin-3-yl)butanoate (1.2 g, 3.33 mmol, 85% yield). ¹H NMR (300 MHz, DMSO-d₆) δ 7.52-7.42 (m, 2H), 7.41-7.26 (m, 3H), 4.30-4.20 (m, 2H), 4.13 (d, J=10.6 Hz, 1H), 3.80 (d, J=7.6 Hz, 1H), 3.07-2.98 (m, 1H), 2.47 (br s, 1H), 2.27 (d, J=13.6 Hz, 1H), 1.43-1.35 (m, 9H), 1.17-1.07 (m, 5H).

Step 6. Pearlman's catalyst Pd(OH)₂ on carbon (1.264 g, 1.799 mmol, 20% w w) was added to a solution of tert-butyl 3-methyl-3-((3S,5R)-2-oxo-5-phenylmorpholin-3-yl)butanoate (1.2 g, 3.60 mmol) in methanol (50 mL)/water (3.13 mL)/TFA (0.625 mL) (40:2.5:0.5, v/v/v). The vessel was purged with H₂ and stirred under H₂ for 24 h. After venting the vessel, the reaction mixture was filtered through Celite, and the filtrate was washed with MeOH. The crude product ((S)-2-amino-5-(tert-butoxy)-3,3-dimethyl-5-oxopentanoic acid (0.83 g, 3.59 mmol, 100% yield)) was concentrated under vacuo. This crude was taken for the next step without further purification. Analytical LC/MS Condition M: 1.13 min, 232.2 [M+H]⁺.

Step 7. The crude product (S)-2-amino-5-(tert-butoxy)-3,3-dimethyl-5-oxopentanoic acid (1 g, 4.32 mmol) dissolved in water (30 mL). Na₂CO₃ (0.916 g, 8.65 mmol) was then added to the above solution. To this solution, Fmoc n-hydroxysuccinimide ester (1.458 g, 4.32 mmol) in dioxane (30 mL) was added drop wise at 0° C. and stirred at room temperature for 16 h. The reaction mixture was acidified to pH −2 by 1N HCl and extracted with EtOAc (50 mL×3), dried over Na₂SO₄, concentrated under vacuo and purified by flash column chromatography on silica gel (EtOAc/petroleum ether, 35 to 39%) to give (S)-2-((((9H-fluoren-9-yl)methoxy)carbonyl)amino)-5-(tert-butoxy)-3,3-dimethyl-5-oxopentanoic acid (0.73 g, 1.567 mmol, 36.2% yield) as a white solid. LCMS, Analytical LC/MS Condition E, MS (ESI) t_{R}=2.135 min, m/z 452.2 [M−H]⁻. ¹H NMR (400 MHz, DMSO-d₆) δ 12.78-12.64 (m, 1H), 7.90 (d, J=7.5 Hz, 2H), 7.77 (dd, J=4.5, 7.0 Hz, 2H), 7.65 (br d, J=9.5 Hz, 1H), 7.46-7.39 (m, 2H), 7.37-7.29 (m, 2H), 4.32-4.15 (m, 4H), 2.39-2.31 (m, 1H), 2.30-2.21 (m, 1H), 1.39 (s, 9H), 1.12-1.00 (m, 6H).

### Preparation of (2S)-2-({[(9H-fluoren-9-yl)methoxy]carbonyl}amino)-3-(morpholin-4-yl)propanoic acid

Step 1 In a 2-L multi-necked round-bottomed flask fitted with a thermo pocket was added (S)-3-amino-2-((tert-butoxycarbonyl)amino)propanoic acid (50 g, 245 mmol), dioxane (500 mL), followed by 1-bromo-2-(2-bromoethoxy)ethane (30.8 mL, 245 mmol) at RT. NaOH (367 mL, 734 mmol) solution was added and the resulting yellow clear solution was heated to 110° C. (external temperature, 85° C. internal temperature) for 12 h. An aliquot of clear solution was subjected to LCMS (Polar method) which showed completion, and then the dioxane was evaporated to get light red solution which was acidified to pH 3. The resulting mixture was concentrated under high vacuum pump (˜4 mbar) at 60° C. to get (S)-2-((tert-butoxycarbonyl)amino)-3-morpholinopropanoic acid (67 g, 244 mmol, 100% yield) pale yellow solid. Analytical LC/MS Condition M: 0.56 min, 275.2 [M+H]⁺.

Step 2 To a stirred suspension of (S)-2-((tert-butoxycarbonyl)amino)-3-morpholinopropanoic acid (100 g, 365 mmol) in dioxane (400 mL) at 0-5° C. was added HCl in dioxane (911 mL, 3645 mmol) slowly over 20 min. The resulting mixture was stirred at RT for 12 h. The volatiles were evaporated to get a pale yellow sticky crude (S)-2-amino-3-morpholinopropanoic acid (16 g), which was taken for next step without further purification. MS (ESI) m/z 175.2 [M+H]⁺.

Step 3 The crude product (S)-2-amino-3-morpholinopropanoic acid (11 g, 63.1 mmol was dissolved in water (250 mL). Na₂CO₃ (13.39 g, 126 mmol) was then added to the above solution. To this solution, Fmoc-N-hydroxysuccinimide ester (21.30 g, 63.1 mmol) was added dropwise at 0° C. and stirred at room temperature for 16 h. The reaction mixture was acidified to pH −2 by 1N HCl and extracted with EtOAc (500 mL×3), dried over Na₂SO₄, concentrated under vacuo, and purified by flash column chromatography on silica gel (petrolium ether/EtOAc, 0-100% then MeOH/CHCl₃ 0-15%) to get (S)-2-((((9H-fluoren-9-yl)methoxy)carbonyl)amino)-3-morpholinopropanoic acid (23 g, 55.9 mmol, 89% yield) as a brown solid. Analytical LC/MS Condition E: 1.43 min, 397.2 [M+H]⁺. ¹H NMR (400 MHz, METHANOL-d₄) δ 7.78 (br d, J=7.5 Hz, 2H), 7.71-7.57 (m, 2H), 7.42-7.34 (m, 2H), 7.34-7.26 (m, 2H), 4.71 (br s, 1H), 4.54-4.32 (m, 2H), 4.29-4.17 (m, 1H), 3.90 (br s, 4H), 3.76-3.62 (m, 1H), 3.58-3.47 (m, 1H), 3.41 (br s, 2H), 3.36-3.32 (m, 2H), 3.31-3.26 (m, 1H).

### Preparation of (2S,3S)-3-{[(tert-butoxy)carbonyl]amino}-2-({[(9H-fluoren-9-yl)methoxy]carbonyl}amino)butanoic acid

To a solution of the benzyl (tert-butoxycarbonyl)-L-threoninate (22 g, 71.1 mmol) in CH₂Cl₂ (600 mL) at −78° C. was sequentially added trifluoromethanesulfonic anhydride (24.08 g, 85 mmol) dropwise and then 2,6-lutidine (10.77 mL, 92 mmol) slowly. After stirring at the same temperature for 1.5 h and monitoring by TLC (Hex:EtOAc 8:2), tetrabutylammonium azide (50.6 g, 178 mmol) was added in portions. After stirring at −78° C. for 1 h, the cooling bath was removed and the reaction mixture was allowed to reach 23° C. for 1.5 h. The reaction was repeated. A saturated aqueous solution of NaHCO₃ was added, and the aqueous phase extracted with EtOAc. The crude product was purified by flash chromatography over silica gel (Hex:EtOAc 95:5 a 9:1) to give benzyl (2S,3S)-3-azido-2-((tert-butoxycarbonyl)amino)butanoate (20 g, 59.8 mmol, 84% yield) as colorless liquid. Analytical LC/MS Condition E: 3.13 min, 333.2 [M−H]⁻.

A solution of benzyl (2S,3S)-3-azido-2-((tert-butoxycarbonyl)amino)butanoate (20 g, 59.8 mmol), dichloromethane (300 mL) and TFA (50 mL, 649 mmol) was stirred for 2 h at 23° C. and then evaporated to dryness to give the corresponding amine. The above amine was redissolved in water (200 mL) and tetrahydrofuran (200 mL). At 0° C., DIPEA (11.49 mL, 65.8 mmol) was added followed by Fmoc chloride (17.02 g, 65.8 mmol). The mixture was warmed up to RT and stirred for 3 h. It was extracted with EtOAc and washed with 0.5 M HCl solution and then brine solution. It was concentrated to get crude liquid. The above crude was purifirf by silica gel column chromatography. The product was eluted with 20% EtOAc in petroleum ether. The fractions were concentrated to get benzyl (2S,3S)-2-((((9H-fluoren-9-yl)methoxy)carbonyl)amino)-3-azidobutanoate (23 g, 50.4 mmol, 84% yield) as a colorless liquid. Analytical LC/MS Condition E: 3.70 min, 479.3 [M+Na]⁺.

Step 3. To a multi-neck round-bottled flask was charged benzyl (2S,3S)-2-((((9H-fluoren-9-yl)methoxy)carbonyl)amino)-3-azidobutanoate (40 g, 88 mmol) in tetrahydrofuran (1200 mL). Pd/C (9.32 g, 8.76 mmol) was added under nitrogen and the reaction was stirred under hydrogen for 12 h. Sodium bicarbonate (11.04 g, 131 mmol) in water 6 (mL) was added followed by Boc-anhydride (30.5 mL, 131 mmol). The mixture was stirred under nitrogen for 12 h. The reaction mass was filtered through a celite bed, and the bed was washed with THF/Water mixture. The mother liquid was concentrated and washed with EtOAc. Then pH of water layer was adjusted to 7-6 using 1.5 N HCl solution. The resulting white solid was extracted with ethyl acetate. The above reaction was repeated three more times. The combined organics were washed with water and brine solution, dried over sodium sulphate, and concentrated to afford (2S,3S)-2-((((9H-fluoren-9-yl)methoxy)carbonyl)amino)-3-((tert-butoxycarbonyl)amino)butanoic acid as a white solid (28 g). This was mixed with a previously obtained batch (8 g) in DCM (200 mL). n-Hexane (1 L) was added to the above solution and sonicated for 2 min. The solids were filtered, rinsed with hexanes and dried overnight to give (2S,3S)-2-((((9H-fluoren-9-yl)methoxy)carbonyl)amino)-3-((tert-butoxycarbonyl)amino)butanoic acid (36 g, 81 mmol, 92% yield) as a white powder. Analytical LC/MS Condition E: 1.90 min, 439.2 [M−H]⁻. ¹H NMR (400 MHz, DMSO-d₆) δ 7.90 (d, J=7.6 Hz, 2H), 7.75 (d, J=7.2 Hz, 2H), 7.43 (t, J=7.2 Hz, 2H), 7.34 (t, J=Hz, 6.71 (br. d. J=7.6 Hz, 1H), 4.29-4.26 (m, 2H), 4.25-4.21 (m, 1H), 3.94-3.90 (m, 1H), 1.37 (s, 9H), 1.02 (d, J=6.8 Hz, 3H). ¹³C NMR (101 Hz, DMSO-d₆) δ 171.9, 156.3, 154.8, 143.7, 140.6, 127.6, 127.0, 125.3, 120.0, 77.7, 65.8, 57.8, 47.0, 46.6, 28.2, 16.2.

### Preparation of (S)-2-((((9H-fluoren-9-yl)methoxy)carbonyl)amino)-2-(1-(((tert-butoxycarbonyl)amino)methyl)cyclopropyl)acetic acid

The compound was obtained following similar procedures of ethyl (S)-5-((tert-butoxycarbonyl)amino)-2-(((S)-mesitylsulfinyl)amino)-3,3-dimethylpentanoate. The synthesis afforded the desired product (0.65 g, 22% yield) as a white solid after purification by flash column chromatography (RediSep, 40 g, SiO₂, 35 to 40% EtOAc:hexanes (compound ELSD active)). Analytical LC/MS Condition E: 2.04 min, 465.2 [M−H]⁻. ¹H NMR (300 MHz, DMSO-d₆) δ 7.90 (d, J=7.6 Hz, 2H), 7.71 (m, 3H), 7.47-7.27 (m, 2H), 6.98-6.71 (m, 2H), 4.30-4.17 (m, 3H), 3.94-3.82 (m, 1H), 3.20-2.90 (m, 2H), 1.44-1.30 (m, 9H), 0.48 (br s, 4H).

### Preparation of (S)-2-((((9H-fluoren-9-yl)methoxy)carbonyl)amino)-2-(1-(tert-butoxycarbonyl)azetidin-3-yl)acetic acid

The compound was obtained following similar procedures of ethyl (S)-5-((tert-butoxycarbonyl)amino)-2-(((S)-mesitylsulfinyl)amino)-3,3-dimethylpentanoate. The synthesis afforded the desired product (2.66 g, 20% yield) as a slightly tan solid after purification by reverse-phase HPLC. Analytical LC/MS Condition E: 1.87 min, 467.2 [M−H]⁻. H NMR (400 MHz, DMSO-d₆) δ 7.89 (d, J=7.6 Hz, 2H), 7.69 (m, 2H), 7.41 (t, J=7.2 Hz, 2H), 7.34-7.31 (m, 2H), 6.71 (br. d. J=7.6 Hz, 1H), 4.29-4.23 (m, 3H), 3.77-3.70 (m, 5H), 2.80 (m, 1H), 1.36 (s, 9H).

### Example 2: Synthesis of Compounds of Formula (I)

### Preparation of Compound 1000

To a 45-mL polypropylene solid-phase reaction vessel was added using Siebber or Rink resin on a 50 μmol scale, and the reaction vessel was placed on the Symphony peptide synthesizer. The following procedures were then performed sequentially: “Symphony Resin-swelling procedure” was followed; “Symphony Single-coupling procedure” was followed with Fmoc-Gly-OH; “Symphony Single-coupling procedure” was followed with Fmoc-Cys(Trt)-OH; “Symphony Single-coupling procedure” was followed with Fmoc-Ser(tBu)-OH; “Symphony Single-coupling procedure” was followed with Fmoc-Val-OH; “Symphony Single-coupling procedure” was followed with Fmoc-Leu-OH; “Symphony Single-coupling procedure” was followed with Fmoc-Arg(Pbf)-OH; “Symphony Single-coupling procedure” was followed with Fmoc-Trp(Boc)-OH; “Symphony Single-coupling procedure” was followed with Fmoc-N-Me-Ala-OH; “Symphony Single-coupling procedure” was followed with Fmoc-Arg(Pbf)-OH; “Symphony double-coupling procedure” was followed with Fmoc-Bip-OH; “Symphony single-coupling procedure” was followed with Fmoc-Val-OH; “Symphony single-coupling procedure” was followed with Fmoc-Trp(Boc)-OH; “Symphony single-coupling procedure” was followed with Fmoc-Asp(tBu)-OH; “Symphony Single-coupling procedure” was followed with Fmoc-Tyr(tBu)-OH; “Symphony Single-coupling procedure” was followed with Fmoc-Phe-OH; “Symphony Chloroacetic Anhydride coupling procedure” was followed; “Global Deprotection Method A” was followed; “Cyclization Method” was followed.

The crude material was purified via preparative LC/MS with the following conditions: Column: XBridge C18, 30×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-65% B over 20 minutes, then a 5-minute hold at 100% B; Flow: 45 mL/min. Fractions containing the desired product were combined and dried via centrifugal evaporation. The yield of the product was 21.4 mg, and its estimated purity by LCMS analysis was 94%.

Analysis condition A: Retention time=1.92 min; ESI-MS(+) m/z [M+2H]²⁺: 998.1.

Analysis condition B: Retention time=1.63 min; ESI-MS(+) m/z [M+H]⁺: 1995.0.

### Preparation of Compound 1001

To a 45-mL polypropylene solid-phase reaction vessel was added using Siebber or Rink resin on a 50 μmol scale, and the reaction vessel was placed on the Symphony X peptide synthesizer. The following procedures were then performed sequentially:

“Symphony X Resin-swelling procedure” was followed; “Symphony X Single-coupling procedure” was followed with Fmoc-Gly-OH; “Symphony X Single-coupling procedure” was followed with Fmoc-Cys(Trt)-OH; “Symphony X Single-coupling procedure” was followed with Fmoc-Ser(tBu)-OH; “Symphony X Single-coupling procedure” was followed with Fmoc-Val-OH; “Symphony X Single-coupling procedure” was followed with Fmoc-Leu-OH; “Symphony X Single-coupling procedure” was followed with Fmoc-Asn(Trt)-OH; “Symphony X Single-coupling procedure” was followed with Fmoc-Asn(Trt)-OH; “Symphony X Single-coupling procedure” was followed with Fmoc-N-Me-Ala-OH; “Symphony X Single-coupling procedure” or “Symphony X double-coupling procedure” was followed with Fmoc-Val-OH; “Symphony X single-coupling procedure” was followed with Fmoc-Bip-OH; “Symphony X single-coupling procedure” was followed with Fmoc-Val-OH; “Symphony X single-coupling procedure” was followed with Fmoc-Trp(Boc)-OH; “Symphony X single-coupling procedure” was followed with Fmoc-Asp(tBu)-OH; “Symphony X Single-coupling procedure” was followed with Fmoc-Tyr(tBu)-OH; “Symphony X Single-coupling procedure” was followed with Fmoc-Phe-OH; “Symphony X Chloroacetic Anhydride coupling procedure” was followed; “Global Deprotection Method A” was followed; “Cyclization Method” was followed.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 38.3 mg, and its estimated purity by LCMS analysis was 99%.

Analysis condition A: Retention time=1.68 min; ESI-MS(+) m/z [M+H]⁺: 1876.2.

Analysis condition B: Retention time=1.78 min; ESI-MS(+) m/z [M+2H]²⁺: 939.2.

### Preparation of Compound 1002

To a 45-mL polypropylene solid-phase reaction vessel was added Rink resin (470 mg, 0.25 mmol), and the reaction vessel was placed on the Prelude peptide synthesizer. The following procedures were then performed sequentially:

“Prelude Resin-swelling procedure” was followed; “Prelude Single-coupling procedure” was followed with Fmoc-Ala-OH; “Prelude Single-coupling procedure” was followed with Fmoc-Cys(Trt)-OH; “Prelude Single-coupling procedure” was followed with Fmoc-Thr(tBu)-OH; “Prelude Single-coupling procedure” was followed with Fmoc-Val-OH; “Prelude Single-coupling procedure” was followed with Fmoc-Leu-OH; “Prelude Single-coupling procedure” was followed with Fmoc-Dab(Boc)-OH; “Prelude Single-coupling procedure” was followed with Fmoc-D-Leu-OH; “Prelude Single-coupling procedure” was followed with Fmoc-NMe-Ala-OH; “Prelude Single-coupling procedure” was followed with Fmoc-Arg(Pbf)-OH; “Prelude Single-coupling procedure” was followed with Fmoc-Phe(4-Br)—OH; “Prelude Single-coupling procedure” was followed with Fmoc-Val-OH; “Prelude Single-coupling procedure” was followed with Fmoc-Trp(Boc)-OH; “Prelude Single-coupling procedure” was followed with Fmoc-Asp(tBu)-OH; “Prelude Single-coupling procedure” was followed with Tyr(tBu)-OH; “Prelude Single-coupling procedure” was followed with Fmoc-Asn(Trt)-OH; The resin was split into 0.050 mmol and was transferred to a Bio-Rad reaction vessel, and “Suzuki Reaction On-resin Procedure” was followed; The resin was transferred to a 45-mL polypropylene solid-phase reaction vessel, and it was placed on the Symphony peptide synthesizer. The following procedures were then performed sequentially: “Symphony Chloroacetic Anhydride coupling procedure” was followed; “Symphony Final rinse and dry procedure” was followed; “Global Deprotection Method A” was followed; “Cyclization Method A” was followed.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 2.8 mg, and its estimated purity by LCMS analysis was 99%.

Analysis condition A: Retention time=1.51 min; ESI-MS(+) m/z [M+2H]²⁺: 968.1.

Analysis condition B: Retention time=1.48 min; ESI-MS(+) m/z [M+2H]²⁺: 967.2.

### Preparation of Compound 1003

To a 45-mL polypropylene solid-phase reaction vessel was added Rink resin (470 mg, 0.25 mmol), and the reaction vessel was placed on the Prelude peptide synthesizer. The following procedures were then performed sequentially:

“Prelude Resin-swelling procedure” was followed; “Prelude Single-coupling procedure” was followed with Fmoc-Ala-OH; “Prelude Single-coupling procedure” was followed with Fmoc-Cys(Trt)-OH; “Prelude Single-coupling procedure” was followed with Fmoc-Thr(tBu)-OH; “Prelude Single-coupling procedure” was followed with Fmoc-Val-OH; “Prelude Single-coupling procedure” was followed with Fmoc-Leu-OH; “Prelude Single-coupling procedure” was followed with Fmoc-Dab(Boc)-OH; “Prelude Single-coupling procedure” was followed with Fmoc-D-Leu-OH; “Prelude Single-coupling procedure” was followed with Fmoc-Ala-OH; The resin was split into 0.050 mmol and was transferred to a Bio-Rad reaction vessel, and “N-Nosylate Formation Procedure” was followed; “N-Alkylation On-resin Procedure Method A” with NHBoc(CH₂)₃OH was followed; “N-Nosylate Removal Procedure” was followed; The resin was transferred to a 45-mL polypropylene solid-phase reaction vessel and it was placed on the Symphony peptide synthesizer. The following procedures were then performed sequentially: “Symphony Single-coupling procedure” was followed with Fmoc-Arg(Pbf)-OH; “Symphony Single-coupling procedure” was followed with Fmoc-Bip-OH; “Symphony Single-coupling procedure” was followed with Fmoc-Val-OH; “Symphony Single-coupling procedure” was followed with Fmoc-Trp(Boc)-OH; “Symphony Single-coupling procedure” was followed with Fmoc-Asp(tBu)-OH; “Symphony Single-coupling procedure” was followed with Tyr(tBu)-OH; “Symphony Single-coupling procedure” was followed with Fmoc-Asn(Trt)-OH; “Symphony Chloroacetic Anhydride coupling procedure” was followed; “Symphony Final rinse and dry procedure” was followed; “Global Deprotection Method A” was followed; “Cyclization Method A” was followed.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 5-55% B over 20 minutes, then a 2-minute hold at 100% B; Flow: 40 mL/min. Fractions containing the desired product were combined and dried via centrifugal evaporation. The yield of the product was 2.4 mg, and its estimated purity by LCMS analysis was 94%.

Analysis condition A: Retention time=1.46 min; ESI-MS(+) m/z [M+2H]²⁺: 972.2.

Analysis condition B: Retention time=1.38 min; ESI-MS(+) m/z [M+2H]²⁺: 972.1.

### Preparation of Compound 1004

To a 45-mL polypropylene solid-phase reaction vessel was added Rink resin (470 mg, 0.25 mmol), and the reaction vessel was placed on the Prelude peptide synthesizer. The following procedures were then performed sequentially: “Prelude Resin-swelling procedure” was followed; “Prelude Single-coupling procedure” was followed with Fmoc-Ala-OH; “Prelude Single-coupling procedure” was followed with Fmoc-Cys(Trt)-OH; “Prelude Single-coupling procedure” was followed with Fmoc-Thr(tBu)-OH; “Prelude Single-coupling procedure” was followed with Fmoc-Val-OH; “Prelude Single-coupling procedure” was followed with Fmoc-Leu-OH; “Prelude Single-coupling procedure” was followed with Fmoc-Dab(Boc)-OH; “Prelude Single-coupling procedure” was followed with Fmoc-D-Leu-OH; “Prelude Single-coupling procedure” was followed with Fmoc-NMe-Ala-OH; “Symphony Single-coupling procedure” was followed with Fmoc-Arg(Pbf)-OH; “Symphony Single-coupling procedure” was followed with Fmoc-Bip-OH; “Symphony Single-coupling procedure” was followed with Fmoc-Val-OH; The resin was transferred to a 45-mL polypropylene solid-phase reaction vessel and it was placed on the Symphony peptide synthesizer. The following procedures were then performed sequentially: “Symphony Single-coupling procedure” was followed with Fmoc-Bzt-OH; “Symphony Single-coupling procedure” was followed with Fmoc-Asp(tBu)-OH; “Symphony Single-coupling procedure” was followed with Tyr(tBu)-OH; “Symphony Single-coupling procedure” was followed with Fmoc-Asn(Trt)-OH; “Symphony Chloroacetic Anhydride coupling procedure” was followed; “Symphony Final rinse and dry procedure” was followed; “Global Deprotection Method A” was followed; “Cyclization Method A” was followed.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 18.2 mg, and its estimated purity by LCMS analysis was 98%.

Analysis condition A: Retention time=1.52 min; ESI-MS(+) m/z [M+2H]²⁺: 960.2.

Analysis condition B: Retention time=1.51 min; ESI-MS(+) m/z [M+2H]²⁺: 960.2.

### Preparation of Compound 1005

To a 45-mL polypropylene solid-phase reaction vessel was added Rink resin (93 mg, 0.05 mmol), and the reaction vessel was placed on the Prelude peptide synthesizer. The following procedures were then performed sequentially:

“Prelude Resin-swelling procedure” was followed; “Prelude Single-coupling procedure” was followed with Fmoc-Ala-OH; “Prelude Single-coupling procedure” was followed with Fmoc-Cys(Trt)-OH; “Prelude Single-coupling procedure” was followed with Fmoc-Thr(tBu)-OH; “Prelude Single-coupling procedure” was followed with Fmoc-Val-OH; “Prelude Single-coupling procedure” was followed with Fmoc-Leu-OH; “Prelude Single-coupling procedure” was followed with Fmoc-Dab(Boc)-OH; “Prelude Single-coupling procedure” was followed with Fmoc-D-Leu-OH; “Prelude Single-coupling procedure” was followed with Fmoc-NMe-Ala-OH; “Prelude Single-coupling procedure” was followed with Fmoc-Val-OH; “Prelude Single-coupling procedure” was followed with Fmoc-Bip-OH; “Prelude Single-coupling procedure” was followed with Fmoc-Val-OH; “Prelude Single-coupling procedure” was followed with Fmoc-Trp(Boc)-OH; “Prelude Single-coupling procedure” was followed with Fmoc-Asp(tBu)-OH; “Prelude Single-coupling procedure” was followed with Tyr(tBu)-OH; “Prelude Single-coupling procedure” was followed with Fmoc-Asn(Trt)-OH; “Prelude Chloroacetic Anhydride coupling procedure” was followed; “Prelude Final rinse and dry procedure” was followed; “Global Deprotection Method A” was followed; “Cyclization Method A” was followed.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 15.2 mg, and its estimated purity by LCMS analysis was 95%.

Analysis condition A: Retention time=1.45 min; ESI-MS(+) m/z [M+2H]²⁺: 923.0.

Analysis condition B: Retention time=1.54 min; ESI-MS(+) m/z [M+2H]²⁺: 923.1.

### Preparation of Compound 1006

Compound 1006 was prepared on a 50 μmol scale. The yield of the product was 21.4 mg, and its estimated purity by LCMS analysis was 94.1%. Analysis condition B: Retention time=1.66 min; ESI-MS(+) m/z [M+H]⁺: 1995.

### Preparation of Compound 1007

Compound 1007 was prepared on a 50 μmol scale. The yield of the product was 41.1 mg, and its estimated purity by LCMS analysis was 93.6%. Analysis condition B: Retention time=1.84 min; ESI-MS(+) m/z [M+H]⁺: 1850.

### Preparation of Compound 1008

Compound 1008 was prepared on a 50 μmol scale. The yield of the product was 4.9 mg, and its estimated purity by LCMS analysis was 94.6%. Analysis condition B: Retention time=1.83 min; ESI-MS(+) m/z [M+H]⁺: 1981.7.

### Preparation of Compound 1009

Compound 1009 was prepared on a 50 μmol scale. The yield of the product was 28.8 mg, and its estimated purity by LCMS analysis was 98.9%. Analysis condition A: Retention time=1.65 min; ESI-MS(+) m/z [M+2H]²⁺: 998.

### Preparation of Compound 1010

Compound 1010 was prepared on a 50 μmol scale. The yield of the product was 44.1 mg, and its estimated purity by LCMS analysis was 98.9%. Analysis condition A: Retention time=1.83 min; ESI-MS(+) m/z [M+2H]²⁺: 1024.1.

### Preparation of Compound 1011

Compound 1011 was prepared on a 50 μmol scale. The yield of the product was 34.1 mg, and its estimated purity by LCMS analysis was 91%. Analysis condition A: Retention time=1.65 min; ESI-MS(+) m/z [M+2H]²⁺: 1047.2.

### Preparation of Compound 1012

Compound 1012 was prepared on a 50 μmol scale. The yield of the product was 31 mg, and its estimated purity by LCMS analysis was 97.2%. Analysis condition B: Retention time=1.73 min; ESI-MS(+) m/z [M+2H]²⁺: 1023.1.

### Preparation of Compound 1013

Compound 1013 was prepare on a 50 μmol scale. The yield of the product was 22.5 mg, and its estimated purity by LCMS analysis was 95.6%. Analysis condition B: Retention time=1.52 min; ESI-MS(+) m/z [M+2H]²⁺: 967.2.

### Preparation of Compound 1014

Compound 1014 was prepared on a 50 μmol scale. The yield of the product was 18.7 mg, and its estimated purity by LCMS analysis was 89%. Analysis condition A: Retention time=1.82 min; ESI-MS(+) m/z [M+2H]²⁺: 1015.1.

### Preparation of Compound 1015

Compound 1015 was prepared on a 50 μmol scale. The yield of the product was 18.5 mg, and its estimated purity by LCMS analysis was 90.8%. Analysis condition A: Retention time=1.75 min; ESI-MS(+) m/z [M+2H]²⁺: 1016.

### Preparation of Compound 1016

Compound 1016 was prepared on a 50 μmol scale. The yield of the product was 12.2 mg, and its estimated purity by LCMS analysis was 97.2%. Analysis condition A: Retention time=1.41 min; ESI-MS(+) m/z [M+2H]²⁺: 960.2.

### Preparation of Compound 1017

Compound 1017 was prepared on a 50 μmol scale. The yield of the product was 34.9 mg, and its estimated purity by LCMS analysis was 98.9%. Analysis condition A: Retention time=1.69 min; ESI-MS(+) m/z [M+2H]²⁺: 1961.

### Preparation of Compound 1018

Compound 1018 was prepared on a 50 μmol scale. The yield of the product was 16.3 mg, and its estimated purity by LCMS analysis was 95.9%. Analysis condition A: Retention time=1.62 min; ESI-MS(+) m/z [M+2H]²⁺: 1005.3.

### Preparation of Compound 1019

Compound 1019 was prepared on a 50 μmol scale. The yield of the product was 21.7 mg, and its estimated purity by LCMS analysis was 99%. Analysis condition B: Retention time=1.38 min; ESI-MS(+) m/z [M+2H]²⁺: 982.2.

### Preparation of Compound 1020

Compound 1020 was prepared on a 50 μmol scale. The yield of the product was 30.7 mg, and its estimated purity by LCMS analysis was 98.4%. Analysis condition B: Retention time=1.5 min; ESI-MS(+) m/z [M+2H]²⁺: 1006.3.

### Preparation of Compound 1021

Compound 1021 was prepared on a 50 μmol scale. The yield of the product was 37.1 mg, and its estimated purity by LCMS analysis was 94.4%. Analysis condition B: Retention time=1.67 min; ESI-MS(+) m/z [M+2H]²⁺: 1018.

### Preparation of Compound 1022

Compound 1022 was prepared on a 50 μmol scale. The yield of the product was 18.1 mg, and its estimated purity by LCMS analysis was 97.5%. Analysis condition A: Retention time=1.4 min; ESI-MS(+) m/z [M+2H]²⁺: 993.2.

### Preparation of Compound 1023

Compound 1023 was prepared on a 50 μmol scale. The yield of the product was 18.9 mg, and its estimated purity by LCMS analysis was 99.1%. Analysis condition B: Retention time=1.45 min; ESI-MS(+) m/z [M+2H]²⁺: 989.

### Preparation of Compound 1024

Compound 1024 was prepared on a 50 μmol scale. The yield of the product was 17.6 mg, and its estimated purity by LCMS analysis was 100%. Analysis condition A: Retention time=1.68 min; ESI-MS(+) m/z [M+H]⁺: 1962.2.

### Preparation of Compound 1025

Compound 1025 was prepared on a 50 μmol scale. The yield of the product was 25.9 mg, and its estimated purity by LCMS analysis was 96.4%. Analysis condition A: Retention time=1.67 min; ESI-MS(+) m/z [M+3H]³⁺: 681.

### Preparation of Compound 1026

Compound 1026 was prepared on a 50 μmol scale. The yield of the product was 36.6 mg, and its estimated purity by LCMS analysis was 100%. Analysis condition A: Retention time=1.66 min; ESI-MS(+) m/z [M+2H]²⁺: 1009.9.

### Preparation of Compound 1027

Compound 1027 was prepared on a 50 μmol scale. The yield of the product was 29.3 mg, and its estimated purity by LCMS analysis was 97.400. Analysis condition A: Retention time=1.79 min; ESI-MS(+) m/z [M+2H]2: +1009.3.

### Preparation of Compound 1028

Compound 1028 was prepared on a 50 μmol scale. The yield of the product was 12.8 mg, and its estimated purity by LCMS analysis was 96.4%. Analysis condition B: Retention time=1.6 min; ESI-MS(+) m/z [M+2H]²⁺: 1016.8.

### Preparation of Compound 1029

Compound 1029 was prepared on a 50 μmol scale. The yield of the product was 20.8 mg, and its estimated purity by LCMS analysis was 98.3%. Analysis condition B: Retention time=1.43 min; ESI-MS(+) m/z [M+H]⁺: 1956.2.

### Preparation of Compound 1030

Compound 1030 was prepared on a 50 μmol scale. The yield of the product was 20.3 mg, and its estimated purity by LCMS analysis was 97%. Analysis condition B: Retention time=1.62 min; ESI-MS(+) m/z [M+2H]²⁺: 960.1.

### Preparation of Compound 1031

Compound 1031 was prepared on a 50 μmol scale. The yield of the product was 19.5 mg, and its estimated purity by LCMS analysis was 97.5%. Analysis condition B: Retention time=1.46 min; ESI-MS(+) m/z [M+3H]³⁺: 663.1.

### Preparation of Compound 1032

Compound 1032 was prepared on a 50 μmol scale. The yield of the product was 29.8 mg, and its estimated purity by LCMS analysis was 92.1%. Analysis condition B: Retention time=1.46, 1.51 min; ESI-MS(+) m/z [M+2H]²⁺: 982.

### Preparation of Compound 1033

Compound 1033 was prepared on a 50 μmol scale. The yield of the product was 21.2 mg, and its estimated purity by LCMS analysis was 100%. Analysis condition A: Retention time=1.51 min; ESI-MS(+) m/z [M+H]⁺: 1942.1.

### Preparation of Compound 1034

Compound 1034 was prepared on a 50 μmol scale. The yield of the product was 31.6 mg, and its estimated purity by LCMS analysis was 97.9%. Analysis condition B: Retention time=1.36 min; ESI-MS(+) m/z [M+H]⁺: 1920.1.

### Preparation of Compound 1035

Compound 1035 was prepared on a 50 μmol scale. The yield of the product was 42.2 mg, and its estimated purity by LCMS analysis was 97.6%. Analysis condition A: Retention time=1.6 min; ESI-MS(+) m/z [M+H]⁺: 1976.1.

### Preparation of Compound 1036

Compound 1036 was prepared on a 50 μmol scale. The yield of the product was 27.4 mg, and its estimated purity by LCMS analysis was 98.4%. Analysis condition B: Retention time=1.26 min; ESI-MS(+) m/z [M+2H]²⁺: 988.4.

### Preparation of Compound 1037

Compound 1037 was prepared on a 50 μmol scale. The yield of the product was 31.5 mg, and its estimated purity by LCMS analysis was 98.3%. Analysis condition B: Retention time=1.6 min; ESI-MS(+) m/z [M+H]⁺: 1942.1.

### Preparation of Compound 1038

Compound 1038 was prepared on a 50 μmol scale. The yield of the product was 65.4 mg, and its estimated purity by LCMS analysis was 100%. Analysis condition A: Retention time=1.53 min: ESI-MS(+) m/z [M+H]⁺: 1900.2.

### Preparation of Compound 1039

Compound 1039 was prepared on a 50 μmol scale. The yield of the product was 31.8 mg, and its estimated purity by LCMS analysis was 99.4%. Analysis condition B: Retention time=1.67 min; ESI-MS(+) m/z [M+H]⁺: 1899.3.

### Preparation of Compound 1040

Compound 1040 was prepared on a 50 μmol scale. The yield of the product was 13.7 mg, and its estimated purity by LCMS analysis was 96.2%. Analysis condition A: Retention time=1.67 min; ESI-MS(+) m/z [M+2H]²⁺: 1028.3.

### Preparation of Compound 1041

Compound 1041 was prepared on a 50 μmol scale. The yield of the product was 8.8 mg, and its estimated purity by LCMS analysis was 96.8%. Analysis condition B: Retention time=1.76 min; ESI-MS(+) m/z [M+2H]²⁺: 1016.4.

### Preparation of Compound 1042

Compound 1042 was prepared on a 50 μmol scale. The yield of the product was 34.6 mg, and its estimated purity by LCMS analysis was 99.2%. Analysis condition B: Retention time=1.53 min; ESI-MS(+) m/z [M+H⁺: 1976.1.

### Preparation of Compound 1043

Compound 1043 was prepared on a 50 μmol scale. The yield of the product was 53.8 mg, and its estimated purity by LCMS analysis was 86.2%. Analysis condition A: Retention time=1.88 min; ESI-MS(+) m/z [M+H]⁺: 1990.

### Preparation of Compound 1044

Compound 1044 was prepared on a 50 μmol scale. The yield of the product was 42.6 mg, and its estimated purity by LCMS analysis was 85.4%. Analysis condition A: Retention time=1.98 min; ESI-MS(+) m/z [M+H]⁺: 1998.8.

### Preparation of Compound 1045

Compound 1045 was prepared on a 50 μmol scale. The yield of the product was 40.6 mg, and its estimated purity by LCMS analysis was 99.1%. Analysis condition B: Retention time=1.52 min; ESI-MS(+) m/z [M+H]⁺: 1956.

### Preparation of Compound 1046

Compound 1046 was prepared on a 500 μmol scale. The yield of the product was 34.3 mg, and its estimated purity by LCMS analysis was 99.4%. Analysis condition B: Retention time=1.37 min; ESI-MS(+) m/z [M+2H]²⁺: 986.

### Preparation of Compound 1047

Compound 1047 was prepared on a 50 μmol scale. The yield of the product was 20.2 mg, and its estimated purity by LCMS analysis was 98.8%. Analysis condition B: Retention time=1.36 min; ESI-MS(+) m/z [M+2H]²⁺: 990.2.

### Preparation of Compound 1048

Compound 1048 was prepared on a 50 μmol scale. The yield of the product was 25 mg, and its estimated purity by LCMS analysis was 98.5%. Analysis condition A: Retention time=1.7 min; ESI-MS(+) m/z [M+H]⁺: 1989.3.

### Preparation of Compound 1049

Compound 1049 was prepared on a 50 μmol scale. The yield of the product was 45.5 mg, and its estimated purity by LCMS analysis was 97.5%. Analysis condition A: Retention time=1.48 min; ESI-MS(+) m/z [M+2H]²⁺: 995.2.

### Preparation of Compound 1050

Compound 1050 was prepared on a 50 μmol scale. The yield of the product was 41.6 mg, and its estimated purity by LCMS analysis was 99.4%. Analysis condition A: Retention time=1.68 min; ESI-MS(+) m/z [M+H]⁺: 1955.2.

### Preparation of Compound 1051

Compound 1051 was prepared on a 50 μmol scale. The yield of the product was 29.7 mg, and its estimated purity by LCMS analysis was 100%. Analysis condition A: Retention time=1.54 min; ESI-MS(+) m/z [M+H]⁺: 1923.2.

### Preparation of Compound 1052

Compound 1052 was prepared on a 50 μmol scale. The yield of the product was 29.5 mg, and its estimated purity by LCMS analysis was 100%. Analysis condition A: Retention time=1.58 min; ESI-MS(+) m/z [M+2H]²⁺: 967.4.

### Preparation of Compound 1053

Compound 1053 was prepared on a 50 μmol scale. The yield of the product was 43.3 mg, and its estimated purity by LCMS analysis was 98.9%. Analysis condition A: Retention time=1.43, 1.46 min; ESI-MS(+) m/z [M+2H]²⁺: 968.18, 968.18.

### Preparation of Compound 1054

Compound 1054 was prepared on a 50 μmol scale. The yield of the product was 51.5 mg, and its estimated purity by LCMS analysis was 92.9%. Analysis condition A: Retention time=1.49 min; ESI-MS(+) m/z [M+H]⁺: 1886.

### Preparation of Compound 1055

Compound 1055 was prepared on a 50 μmol scale. The yield of the product was 21.3 mg, and its estimated purity by LCMS analysis was 86.4%. Analysis condition B: Retention time=1.62 min; ESI-MS(+) m/z [M+2H]²⁺: 1017.

### Preparation of Compound 1056

Compound 1056 was prepared on a 50 μmol scale. The yield of the product was 11.6 mg, and its estimated purity by LCMS analysis was 99.1%. Analysis condition B: Retention time=1.45 min; ESI-MS(+) m/z [M+H]⁺: 1914.4.

### Preparation of Compound 1057

Compound 1057 was prepared on a 50 μmol scale. The yield of the product was 9.1 mg, and its estimated purity by LCMS analysis was 100%. Analysis condition A: Retention time=1.75 min; ESI-MS(+) m/z [M+2H]²⁺: 1024.3.

### Preparation of Compound 1058

Compound 1058 was prepared on a 50 μmol scale. The yield of the product was 60.9 mg, and its estimated purity by LCMS analysis was 91.1%. Analysis condition A: Retention time=1.56 min; ESI-MS(+) m/z [M+H]⁺: 1976.9.

### Preparation of Compound 1059

Compound 1059 was prepared on a 50 μmol scale. The yield of the product was 55.5 mg, and its estimated purity by LCMS analysis was 98.7%. Analysis condition B: Retention time=1.64 min; ESI-MS(+) m/z [M+2H]²⁺: 1005.1.

### Preparation of Compound 1060

Compound 1060 was prepared on a 50 μmol scale. The yield of the product was 69.7 mg, and its estimated purity by LCMS analysis was 97.1%. Analysis condition A: Retention time=1.67 min; ESI-MS(+) m/z [M+H]⁺: 1975.

### Preparation of Compound 1061

Compound 1061 was prepared on a 50 μmol scale. The yield of the product was 37.6 mg, and its estimated purity by LCMS analysis was 97%. Analysis condition A: Retention time=1.77 min; ESI-MS(+) m/z [M+2H]²⁺: 1008.2.

### Preparation of Compound 1062

Compound 1062 was prepared on a 50 μmol scale. The yield of the product was 62.1 mg, and its estimated purity by LCMS analysis was 95.9%. Analysis condition B: Retention time=1.59 min; ESI-MS(+) m/z [M+2H]²⁺: 1004.3.

### Preparation of Compound 1063

Compound 1063 was prepared on a 50 μmol scale. The yield of the product was 43.9 mg, and its estimated purity by LCMS analysis was 98.7%. Analysis condition A: Retention time=1.62, 1.68 min; ESI-MS(+) m/z [M+H]⁺: 1989.02, 1989.02.

### Preparation of Compound 1064

Compound 1064 was prepared on a 50 μmol scale. The yield of the product was 62.5 mg, and its estimated purity by LCMS analysis was 98.7%. Analysis condition A: Retention time=1.7 min; ESI-MS(+) m/z [M+2H]²⁺: 988.1.

### Preparation of Compound 1065

Compound 1065 was prepared on a 50 μmol scale. The yield of the product was 49.9 mg, and its estimated purity by LCMS analysis was 97.7%. Analysis condition A: Retention time=1.85 min; ESI-MS(+) m/z [M+2H]²⁺: 1019.1.

### Preparation of Compound 1066

Compound 1066 was prepared on a 50 μmol scale. The yield of the product was 12.1 mg, and its estimated purity by LCMS analysis was 100%. Analysis condition A: Retention time=1.69 min; ESI-MS(+) m/z [M+H]⁺: 1990.2.

### Preparation of Compound 1067

Compound 1067 was prepared on a 50 μmol scale. The yield of the product was 15 mg, and its estimated purity by LCMS analysis was 94.1%. Analysis condition A: Retention time=1.47 min; ESI-MS(+) m/z [M+2H]²⁺: 1024.

### Preparation of Compound 1068

Compound 1068 was prepared on a 50 μmol scale. The yield of the product was 17.8 mg, and its estimated purity by LCMS analysis was 100%. Analysis condition A: Retention time=1.73 min; ESI-MS(+) m/z [M+2H]²⁺: 1013.2.

### Preparation of Compound 1069

Compound 1069 was prepared on a 50 μmol scale. The yield of the product was 18.7 mg, and its estimated purity by LCMS analysis was 93.8%. Analysis condition B: Retention time=1.56 min; ESI-MS(+) m/z [M+2H]²⁺: 981.1.

### Preparation of Compound 1070

Compound 1070 was prepared on a 50 μmol scale. The yield of the product was 54.5 mg, and its estimated purity by LCMS analysis was 97.5%. Analysis condition B: Retention time=1.76 min; ESI-MS(+) m/z [M+2H]²⁺: 1029.1.

### Preparation of Compound 1071

Compound 1071 was prepared on a 50 μmol scale. The yield of the product was 35.1 mg, and its estimated purity by LCMS analysis was 100%. Analysis condition A: Retention time=1.75 min; ESI-MS(+) m/z [M+2H]²⁺: 1021.

### Preparation of Compound 1072

Compound 1072 was prepared on a 50 μmol scale. The yield of the product was 46.9 mg, and its estimated purity by LCMS analysis was 98.9%. Analysis condition B: Retention time=1.4 min; ESI-MS(+) m/z [M+2H]²⁺: 1012.2.

### Preparation of Compound 1073

Compound 1073 was prepared on a 50 μmol scale. The yield of the product was 46.9 mg, and its estimated purity by LCMS analysis was 99%. Analysis condition A: Retention time=1.69 min; ESI-MS(+) m/z [M+2H]²⁺: 1030.1.

### Preparation of Compound 1074

Compound 1074 was prepared on a 50 μmol scale. The yield of the product was 47.3 mg, and its estimated purity by LCMS analysis was 97.7%. Analysis condition B: Retention time=1.62 min; ESI-MS(+) m/z [M+2H]²⁺: 1034.3.

### Preparation of Compound 1075

Compound 1075 was prepared on a 50 μmol scale. The yield of the product was 32.4 mg, and its estimated purity by LCMS analysis was 100%. Analysis condition A: Retention time=1.8 min; ESI-MS(+) m/z [M+2H]²⁺: 1046.2.

### Preparation of Compound 1076

Compound 1076 was prepared on a 50 μmol scale. The yield of the product was 37.7 mg, and its estimated purity by LCMS analysis was 99.2%. Analysis condition A: Retention time=1.81 min; ESI-MS(+) m/z [M+2H]²⁺: 1015.

### Preparation of Compound 1077

Compound 1077 was prepared on a 50 μmol scale. The yield of the product was 1.2 mg, and its estimated purity by LCMS analysis was 98.8%. Analysis condition A: Retention time=1.52 min; ESI-MS(+) m/z [M+2H]²⁺: 1010.2.

### Preparation of Compound 1078

Compound 1078 was prepared on a 50 μmol scale. The yield of the product was 24.5 mg, and its estimated purity by LCMS analysis was 97.3%. Analysis condition A: Retention time=1.39 min; ESI-MS(+) m/z [M+2H]²⁺: 996.1.

### Preparation of Compound 1079

Compound 1079 was prepared on a 50 μmol scale. The yield of the product was 12.3 mg, and its estimated purity by LCMS analysis was 95.9%. Analysis condition A: Retention time=1.7 min; ESI-MS(+) m/z [M+2H]²⁺: 1004.

### Preparation of Compound 1080

Compound 1080 was prepared on a 50 μmol scale. The yield of the product was 57.4 mg, and its estimated purity by LCMS analysis was 98.5%. Analysis condition B: Retention time=1.79 min; ESI-MS(+) m/z [M+3H]³⁺: 664.4.

### Preparation of Compound 1081

Compound 1081 was prepared on a 50 μmol scale. The yield of the product was 15.3 mg, and its estimated purity by LCMS analysis was 100%. Analysis condition A: Retention time=1.91 min; ESI-MS(+) m/z [M+2H]²⁺: 1004.

### Preparation of Compound 1082

Compound 1082 was prepared on a 50 μmol scale. The yield of the product was 39.1 mg, and its estimated purity by LCMS analysis was 98.4%. Analysis condition B: Retention time=1.49 min; ESI-MS(+) m/z [M+H]⁺: 1988.2.

### Preparation of Compound 1083

Compound 1083 was prepare on a 50 μmol scale. The yield of the product was 45.5 mg, and its estimated purity by LCMS analysis was 98.6%. Analysis condition B: Retention time=1.47 min; ESI-MS(+) m/z [M+2H]²⁺: 1002.1.

### Preparation of Compound 1084

Compound 1084 was prepared on a 50 μmol scale. The yield of the product was 43.6 mg, and its estimated purity by LCMS analysis was 96.9%. Analysis condition B: Retention time=1.46 min; ESI-MS(+) m/z [M+H]⁺: 1989.2.

### Preparation of Compound 1085

Compound 1085 was prepared on a 50 μmol scale. The yield of the product was 57.2 mg, and its estimated purity by LCMS analysis was 96.3%. Analysis condition A: Retention time=1.56 min; ESI-MS(+) m/z [M+2H]²⁺: 987.7.

### Preparation of Compound 1086

Compound 1086 was prepared on a 50 μmol scale. The yield of the product was 40.7 mg, and its estimated purity by LCMS analysis was 99.2%. Analysis condition A: Retention time=1.76 min; ESI-MS(+) m/z [M+2H]²⁺: 1052.1.

### Preparation of Compound 1087

Compound 1087 was prepared on a 50 μmol scale. The yield of the product was 30.4 mg, and its estimated purity by LCMS analysis was 93.7%. Analysis condition B: Retention time=1.69 min; ESI-MS(+) m/z [M+2H]²⁺: 1020.1.

### Preparation of Compound 1088

Compound 1088 was prepared on a 50 μmol scale. The yield of the product was 24.1 mg, and its estimated purity by LCMS analysis was 99.1%. Analysis condition B: Retention time=1.86 min; ESI-MS(+) m/z [M+2H]²⁺: 1015.4.

### Preparation of Compound 1089

Compound 1089 was prepared on a 50 μmol scale. The yield of the product was 23.8 mg, and its estimated purity by LCMS analysis was 100%. Analysis condition A: Retention time=1.63 min; ESI-MS(+) m/z [M+2H]²⁺: 1020.4.

### Preparation of Compound 1090

Compound 1090 was prepared on a 50 μmol scale. The yield of the product was 24.2 mg, and its estimated purity by LCMS analysis was 97.7%. Analysis condition A: Retention time=1.69 min; ESI-MS(+) m/z [M+H]⁺: 1948.2.

### Preparation of Compound 1091

Compound 1091 was prepared on a 50 μmol scale. The yield of the product was 24.4 mg, and its estimated purity by LCMS analysis was 100%. Analysis condition A: Retention time=1.82 min; ESI-MS(+) m/z [M+2H]²⁺: 1002.

### Preparation of Compound 1092

Compound 1092 was prepared on a 50 μmol scale. The yield of the product was 27.1 mg, and its estimated purity by LCMS analysis was 96.6%. Analysis condition B: Retention time=1.8 min; ESI-MS(+) m/z [M+2H]²⁺: 1075.

### Preparation of Compound 1093

Compound 1093 was prepared on a 50 μmol scale. The yield of the product was 16.8 mg, and its estimated purity by LCMS analysis was 100%. Analysis condition A: Retention time=1.87 min; ESI-MS(+) m/z [M+2H]²⁺: 1121.1.

### Preparation of Compound 1094

Compound 1094 was prepared on a 50 μmol scale. The yield of the product was 38.4 mg, and its estimated purity by LCMS analysis was 93.3%. Analysis condition B: Retention time=1.65 min; ESI-MS(+) m/z [M+2H]²⁺: 1024.1.

### Preparation of Compound 1095

Compound 1095 was prepared on a 50 μmol scale. The yield of the product was 10.6 mg, and its estimated purity by LCMS analysis was 91.6%. Analysis condition B: Retention time=1.76 min; ESI-MS(+) m/z [M+2H]²⁺: 1018.1.

### Preparation of Compound 1096

Compound 1096 was prepared on a 50 μmol scale. The yield of the product was 37 mg, and its estimated purity by LCMS analysis was 95.1%. Analysis condition A: Retention time=1.54 min; ESI-MS(+) m/z [M+2H]²⁺: 996.2.

### Preparation of Compound 1097

Compound 1097 was prepared on a 50 μmol scale. The yield of the product was 50.2 mg, and its estimated purity by LCMS analysis was 93.8%. Analysis condition A: Retention time=1.64 min; ESI-MS(+) m/z [M+2H]²⁺: 1003.2.

### Preparation of Compound 1098

Compound 1098 was prepared on a 50 μmol scale. The yield of the product was 42.5 mg, and its estimated purity by LCMS analysis was 91.2%. Analysis condition A: Retention time=1.77 min; ESI-MS(+) m/z [M+2H]²⁺: 1012.1.

### Preparation of Compound 1099

Compound 1099 was prepared on a 50 μmol scale. The yield of the product was 38.7 mg, and its estimated purity by LCMS analysis was 100%. Analysis condition A: Retention time=1.62 min; ESI-MS(+) m/z [M+2H]²⁺: 1009.1.

### Preparation of Compound 1100

Compound 1100 was prepared on a 50 μmol scale. The yield of the product was 70.1 mg, and its estimated purity by LCMS analysis was 91.3%. Analysis condition B: Retention time=1.76 min; ESI-MS(+) m/z [M+2H]²⁺: 1000.2.

### Preparation of Compound 1101

Compound 1101 was prepared on a 50 μmol scale. The yield of the product was 50 mg, and its estimated purity by LCMS analysis was 100%. Analysis condition A: Retention time=1.75 min; ESI-MS(+) m/z [M+2H]²⁺: 988.1.

### Preparation of Compound 1102

Compound 1102 was prepared on a 50 μmol scale. The yield of the product was 52.2 mg, and its estimated purity by LCMS analysis was 95.9%. Analysis condition B: Retention time=1.71 min; ESI-MS(+) m/z [M+2H]²⁺: 1035.1.

### Preparation of Compound 1103

Compound 1103 was prepared on a 50 μmol scale. The yield of the product was 51.4 mg, and its estimated purity by LCMS analysis was 98.3%. Analysis condition B: Retention time=1.74 min; ESI-MS(+) m/z [M+2H]²⁺: 1030.1.

### Preparation of Compound 1104

Compound 1104 was prepared on a 50 μmol scale. The yield of the product was 26.6 mg, and its estimated purity by LCMS analysis was 95%. Analysis condition B: Retention time=1.43 min; ESI-MS(+) m/z [M+3H]³⁺: 675.3.

### Preparation of Compound 1105

Compound 1105 was prepared on a 50 μmol scale. The yield of the product was 27.9 mg, and its estimated purity by LCMS analysis was 95.2%. Analysis condition A: Retention time=1.64 min; ESI-MS(+) m/z [M+2H]²⁺: 1002.8.

### Preparation of Compound 1106

Compound 1106 was prepared on a 50 μmol scale. The yield of the product was 2.8 mg, and its estimated purity by LCMS analysis was 89.8%. Analysis condition A: Retention time=1.75 min; ESI-MS(+) m/z [M+2H]²⁺: 1002.2.

### Preparation of Compound 1107

Compound 1107 was prepared on a 50 μmol scale. The yield of the product was 40.6 mg, and its estimated purity by LCMS analysis was 96.3%. Analysis condition A: Retention time=1.63 min; ESI-MS(+) m/z [M+2H]²⁺: 1025.1.

### Preparation of Compound 1108

Compound 1108 was prepared on a 50 μmol scale. The yield of the product was 34.6 mg, and its estimated purity by LCMS analysis was 97.9%. Analysis condition B: Retention time=1.67 min; ESI-MS(+) m/z [M+2H]²⁺: 1027.3.

### Preparation of Compound 1109

Compound 1109 was prepared on a 50 μmol scale. The yield of the product was 5 mg, and its estimated purity by LCMS analysis was 95.8%. Analysis condition A: Retention time=1.7 min; ESI-MS(+) m/z [M+2H]²⁺: 1058.3.

### Preparation of Compound 1110

Compound was prepare on a 50 μmol scale. The yield of the product was 17 mg, and its estimated purity by LCMS analysis was 85.5%. Analysis condition A: Retention time=1.38, 1.43 min; ESI-MS(+) m/z [M+2H]²⁺: 1025.7.

### Preparation of Compound 1111

Compound 1111 was prepared on a 50 μmol scale. The yield of the product was 22.3 mg, and its estimated purity by LCMS analysis was 89.5%. Analysis condition B: Retention time=1.66 min; ESI-MS(+) m/z [M+2H]²⁺: 994.2.

### Preparation of Compound 1112

Compound 1112 was prepared on a 50 μmol scale. The yield of the product was 23.2 mg, and its estimated purity by LCMS analysis was 98.9%. Analysis condition B: Retention time=1.56 min; ESI-MS(+) m/z [M+2H]²⁺: 995.1.

### Preparation of Compound 1113

Compound 1113 was prepared on a 50 μmol scale. The yield of the product was 12 mg, and its estimated purity by LCMS analysis was 100%. Analysis condition A: Retention time=1.6 min; ESI-MS(+) m/z [M+2H]²⁺: 988.2.

### Preparation of Compound 1114

Compound 1114 was prepared on a 50 μmol scale. The yield of the product was 14 mg, and its estimated purity by LCMS analysis was 97.2%. Analysis condition A: Retention time=1.57 min; ESI-MS(+) m/z [M+2H]²⁺: 981.

### Preparation of Compound 1115

Compound 1115 was prepared on a 50 μmol scale. The yield of the product was 23.7 mg, and its estimated purity by LCMS analysis was 98.5%. Analysis condition B: Retention time=1.78 min; ESI-MS(+) m/z [M+2H]²⁺: 1015.2.

### Preparation of Compound 1116

Compound 1116 was prepared on a 50 μmol scale. The yield of the product was 37.7 mg, and its estimated purity by LCMS analysis was 97.4%. Analysis condition A: Retention time=1.72 min; ESI-MS(+) m/z [M+2H]²⁺: 1002.4.

### Preparation of Compound 1117

Compound 1117 was prepared on a 50 μmol scale. The yield of the product was 26.3 mg, and its estimated purity by LCMS analysis was 98.3%. Analysis condition A: Retention time=1.7 min; ESI-MS(+) m/z [M+2H]²⁺: 1002.4.

### Preparation of Compound 1118

Compound 1118 was prepared on a 50 μmol scale. The yield of the product was 42.7 mg, and its estimated purity by LCMS analysis was 98.8%. Analysis condition B: Retention time=1.68 min; ESI-MS(+) m/z [M+3H]³⁺: 668.2.

### Preparation of Compound 1119

Compound 1119 was prepared on a 50 μmol scale. The yield of the product was 32.8 mg, and its estimated purity by LCMS analysis was 97.7%. Analysis condition A: Retention time=1.59 min; ESI-MS(+) m/z [M+2H]²⁺: 974.

### Preparation of Compound 1120

Compound 1120 was prepared on a 50 μmol scale. The yield of the product was 16.9 mg, and its estimated purity by LCMS analysis was 97.1%. Analysis condition B: Retention time=1.72 min; ESI-MS(+) m/z [M+3H]³⁺: 680.

### Preparation of Compound 1121

Compound 1121 was prepared on a 50 μmol scale. The yield of the product was 27.2 mg, and its estimated purity by LCMS analysis was 99.3%. Analysis condition B: Retention time=1.56 min; ESI-MS(+) m/z [M+2H]²⁺: 947.1.

### Preparation of Compound 1122

Compound 1122 was prepared on a 50 μmol scale. The yield of the product was 36.7 mg, and its estimated purity by LCMS analysis was 97%. Analysis condition A: Retention time=1.91 min; ESI-MS(+) m/z [M+2H]²⁺: 991.1.

### Preparation of Compound 1123

Compound 1123 was prepared on a 50 μmol scale. The yield of the product was 30.2 mg, and its estimated purity by LCMS analysis was 94.4%. Analysis condition A: Retention time=1.66 min; ESI-MS(+) m/z [M+2H]²⁺: 975.1.

### Preparation of Compound 1124

Compound 1124 was prepared on a 50 μmol scale. The yield of the product was 59.9 mg, and its estimated purity by LCMS analysis was 96%. Analysis condition A: Retention time=1.87 min; ESI-MS(+) m/z [M+2H]²⁺: 1019.2.

### Preparation of Compound 1125

Compound 1125 was prepared on a 50 μmol scale. The yield of the product was 35.4 mg, and its estimated purity by LCMS analysis was 97.7%. Analysis condition A: Retention time=1.64 min; ESI-MS(+) m/z [M+2H]²⁺: 983.

### Preparation of Compound 1126

Compound 1126 was prepared on a 50 μmol scale. The yield of the product was 30.9 mg, and its estimated purity by LCMS analysis was 98%. Analysis condition A: Retention time=1.76 min; ESI-MS(+) m/z [M+H]⁺: 1968.2.

### Preparation of Compound 1127

Compound 1127 was prepared on a 50 μmol scale. The yield of the product was 37.6 mg, and its estimated purity by LCMS analysis was 95%. Analysis condition A: Retention time=1.52 min; ESI-MS(+) m/z [M+2H]²⁺: 976.4.

### Preparation of Compound 1128

Compound 1128 was prepared on a 50 μmol scale. The yield of the product was 48.4 mg, and its estimated purity by LCMS analysis was 98.6%. Analysis condition B: Retention time=1.41 min; ESI-MS(+) m/z [M+2H]²⁺: 958.

### Preparation of Compound 1129

Compound 1129 was prepared on a 50 μmol scale. The yield of the product was 39.2 mg, and its estimated purity by LCMS analysis was 98.3%. Analysis condition A: Retention time=1.51 min; ESI-MS(+) m/z [M+2H]²⁺: 961.5.

### Preparation of Compound 1130

Compound 1130 was prepared on a 50 μmol scale. The yield of the product was 38 mg, and its estimated purity by LCMS analysis was 96.5%. Analysis condition B: Retention time=1.47 min; ESI-MS(+) m/z [M+H]⁺: 1908.3.

### Preparation of Compound 1131

Compound 1131 was prepared on a 50 μmol scale. The yield of the product was 28.2 mg, and its estimated purity by LCMS analysis was 98%. Analysis condition A: Retention time=1.46 min; ESI-MS(+) m/z [M+H]⁺: 1889.1.

### Preparation of Compound 1132

Compound 1132 was prepared on a 50 μmol scale. The yield of the product was 27.6 mg, and its estimated purity by LCMS analysis was 98%. Analysis condition B: Retention time=1.91 min; ESI-MS(+) m/z [M+2H]²⁺: 1006.1.

### Preparation of Compound 1133

Compound 1133 was prepared on a 50 μmol scale. The yield of the product was 48.1 mg, and its estimated purity by LCMS analysis was 87.4%. Analysis condition B: Retention time=1.87 min; ESI-MS(+) m/z [M+2H]²⁺: 1027.1.

### Preparation of Compound 1134

Compound 1134 was prepared on a 50 μmol scale. The yield of the product was 31 mg, and its estimated purity by LCMS analysis was 98.7%. Analysis condition A: Retention time=1.7 min: ESI-MS(+) m/z [M+H]⁺: 1907.

### Preparation of Compound 1135

Compound 1135 was prepared on a 100 μmol scale. The yield of the product was 35.1 mg, and its estimated purity by LCMS analysis was 92.1%. Analysis condition B: Retention time=1.67 min; ESI-MS(+) m/z [M+2H]²⁺: 1011.1.

### Preparation of Compound 1136

Compound 1136 was prepared on a 50 μmol scale. The yield of the product was 30.6 mg, and its estimated purity by LCMS analysis was 100%. Analysis condition B: Retention time=1.24 min; ESI-MS(+) m/z [M+3H]³⁺: 638.

### Preparation of Compound 1137

Compound 1137 was prepared on a 50 μmol scale. The yield of the product was 39.6 mg, and its estimated purity by LCMS analysis was 97.9%. Analysis condition B: Retention time=1.31 min; ESI-MS(+) m/z [M+2H]²⁺: 938.

### Preparation of Compound 1138

Compound 1138 was prepared on a 50 μmol scale. The yield of the product was 30.6 mg, and its estimated purity by LCMS analysis was 91.3%. Analysis condition B: Retention time=1.51 min; ESI-MS(+) m/z [M+H]⁺: 1958.

### Preparation of Compound 1139

Compound 1139 was prepared on a 50 μmol scale. The yield of the product was 18.8 mg, and its estimated purity by LCMS analysis was 97.7%. Analysis condition A: Retention time=1.37 min; ESI-MS(+) m/z [M+2H]²⁺: 972.2.

### Preparation of Compound 1140

Compound 1140 was prepared on a 50 μmol scale. The yield of the product was 28.1 mg, and its estimated purity by LCMS analysis was 97.8%. Analysis condition B: Retention time=1.55, 1.58 min; ESI-MS(+) m/z [M+2H]²⁺: 1004.1.

### Preparation of Compound 1141

Compound 1141 was prepared on a 50 μmol scale. The yield of the product was 35.1 mg, and its estimated purity by LCMS analysis was 94.6%. Analysis condition B: Retention time=1.69 min; ESI-MS(+) m/z [M+2H]²⁺: 982.

### Preparation of Compound 1142

Compound 1142 was prepared on a 50 μmol scale. The yield of the product was 33.4 mg, and its estimated purity by LCMS analysis was 100%. Analysis condition A: Retention time=1.38 min; ESI-MS(+) m/z [M+3H]³⁺: 633.2.

### Preparation of Compound 1143

Compound 1143 was prepared on a 50 μmol scale. The yield of the product was 33.5 mg, and its estimated purity by LCMS analysis was 100%. Analysis condition A: Retention time=1.91 min; ESI-MS(+) m/z [M+H]⁺: 1997.2.

### Preparation of Compound 1144

Compound 1144 was prepared on a 50 μmol scale. The yield of the product was 44 mg, and its estimated purity by LCMS analysis was 89.4%. Analysis condition A: Retention time=1.44 min; ESI-MS(+) m/z [M+2H]²⁺: 969.5.

### Preparation of Compound 1145

Compound 1145 was prepared on a 50 μmol scale. The yield of the product was 9.7 mg, and its estimated purity by LCMS analysis was 99.4%. Analysis condition B: Retention time=1.73 min; ESI-MS(+) m/z [M+2H]²⁺: 990.1.

### Preparation of Compound 1146

Compound 1146 was prepared on a 50 μmol scale. The yield of the product was 31.3 mg, and its estimated purity by LCMS analysis was 100%. Analysis condition A: Retention time=1.43 min; ESI-MS(+) m/z [M+2H]²⁺: 943.2.

### Preparation of Compound 1147

Compound 1147 was prepared on a 50 μmol scale. The yield of the product was 26.8 mg, and its estimated purity by LCMS analysis was 99%. Analysis condition B: Retention time=1.29 min; ESI-MS(+) m/z [M+3H]³⁺: 640.3.

### Preparation of Compound 1148

Compound 1148 was prepared on a 50 μmol scale. The yield of the product was 14.4 mg, and its estimated purity by LCMS analysis was 99%. Analysis condition A: Retention time=1.35 min; ESI-MS(+) m/z [M+3H]³⁺: 666.1.

### Preparation of Compound 1149

Compound 1149 was prepared on a 50 μmol scale. The yield of the product was 16.8 mg, and its estimated purity by LCMS analysis was 96.8%. Analysis condition A: Retention time=1.39 min; ESI-MS(+) m/z [M+3H]³⁺: 655.4.

### Preparation of Compound 1150

Compound 1150 was prepared on a 50 μmol scale. The yield of the product was 17.7 mg, and its estimated purity by LCMS analysis was 100%. Analysis condition A: Retention time=1.41 min; ESI-MS(+) m/z [M+2H]²⁺: 991.2.

### Preparation of Compound 1151

Compound 1151 was prepared on a 50 μmol scale. The yield of the product was 13.7 mg, and its estimated purity by LCMS analysis was 100%. Analysis condition B: Retention time=1.41 min; ESI-MS(+) m/z [M+2H]²⁺: 976.1.

### Preparation of Compound 1152

Compound 1152 was prepared on a 50 μmol scale. The yield of the product was 19.1 mg, and its estimated purity by LCMS analysis was 95.6%. Analysis condition A: Retention time=1.39 min; ESI-MS(+) m/z [M+2H]²⁺: 987.2.

### Preparation of Compound 1153

Compound 1153 was prepared on a 50 μmol scale. The yield of the product was 26 mg, and its estimated purity by LCMS analysis was 100%. Analysis condition A: Retention time=1.64 min; ESI-MS(+) m/z [M+H]⁺: 1990.

### Preparation of Compound 1154

Compound 1154 was prepared on a 50 μmol scale. The yield of the product was 20.5 mg, and its estimated purity by LCMS analysis was 100%. Analysis condition A: Retention time=1.68 min; ESI-MS(+) m/z [M+H]⁺: 1960.

### Preparation of Compound 1155

Compound 1155 was prepared on a 50 μmol scale. The yield of the product was 9.6 mg, and its estimated purity by LCMS analysis was 100%. Analysis condition A: Retention time=1.68 min; ESI-MS(+) m/z [M+H]⁺: 1979.

### Preparation of Compound 1156

Compound 1156 was prepared on a 50 μmol scale. The yield of the product was 18.2 mg, and its estimated purity by LCMS analysis was 99.1%. Analysis condition B: Retention time=1.69 min; ESI-MS(+) m/z [M+H]⁺: 1975.

### Preparation of Compound 1157

Compound 1157 was prepared on a 50 μmol scale. The yield of the product was 14 mg, and its estimated purity by LCMS analysis was 99%. Analysis condition B: Retention time=1.24 min; ESI-MS(+) m/z [M+3H]³⁺: 637.1.

### Preparation of Compound 1158

Compound 1158 was prepared on a 50 μmol scale. The yield of the product was 16.5 mg, and its estimated purity by LCMS analysis was 100%. Analysis condition A: Retention time=1.5 min; ESI-MS(+) m/z [M+2H]²⁺: 997.

### Preparation of Compound 1159

Compound 1159 was prepared on a 50 μmol scale. The yield of the product was 5.4 mg, and its estimated purity by LCMS analysis was 100%. Analysis condition A: Retention time=1.58 min; ESI-MS(+) m/z [M+H]⁺: 1923.

### Preparation of Compound 1160

Compound 1160 was prepared on a 50 μmol scale. The yield of the product was 15.7 mg, and its estimated purity by LCMS analysis was 99.4%. Analysis condition B: Retention time=1.59 min; ESI-MS(+) m/z [M+H]⁺: 1944.9.

### Preparation of Compound 1161

Compound 1161 was prepared on a 50 μmol scale. The yield of the product was 31.2 mg, and its estimated purity by LCMS analysis was 97.4%. Analysis condition B: Retention time=1.35 min; ESI-MS(+) m/z [M+2H]²⁺: 1012.9.

### Preparation of Compound 1162

Compound 1162 was prepared on a 50 μmol scale. The yield of the product was 32.2 mg, and its estimated purity by LCMS analysis was 100%. Analysis condition A: Retention time=1.61 min; ESI-MS(+) m/z [M+3H]³⁺: 659.2.

### Preparation of Compound 1163

Compound 1163 was prepared on a 100 μmol scale. The yield of the product was 19.7 mg, and its estimated purity by LCMS analysis was 100%. Analysis condition A: Retention time=1.67 min; ESI-MS(+) m/z [M+H]⁺: 1977.8.

### Preparation of Compound 1164

Compound 1164 was prepared on a 100 μmol scale. The yield of the product was 19.8 mg, and its estimated purity by LCMS analysis was 100%. Analysis condition A: Retention time=1.66 min; ESI-MS(+) m/z [M+H]⁺: 1963.8.

### Preparation of Compound 1165

Compound 1165 was prepare on a 100 μmol scale. The yield of the product was 9 mg, and its estimated purity by LCMS analysis was 100%. Analysis condition B: Retention time=1.39 min; ESI-MS(+) m/z [M+2H]²⁺: 987.1.

### Preparation of Compound 1166

Compound 1166 was prepared on a 50 μmol scale. The yield of the product was 8 mg, and its estimated purity by LCMS analysis was 99.4%. Analysis condition B: Retention time=1.49 min; ESI-MS(+) m/z [M+2H]²⁺: 978.8.

### Preparation of Compound 1167

Compound 1167 was prepared on a 50 μmol scale. The yield of the product was 6.3 mg, and its estimated purity by LCMS analysis was 91.4%. Analysis condition A: Retention time=1.6, 1.63 min; ESI-MS(+) m/z [M+2H]²⁺: 1019.2.

### Preparation of Compound 1168

Compound 1168 was prepared on a 50 μmol scale. The yield of the product was 18.3 mg, and its estimated purity by LCMS analysis was 97.9%. Analysis condition A: Retention time=1.62 min; ESI-MS(+) m/z [M+2H]²⁺: 1010.9.

### Preparation of Compound 1169

Compound 1169 was prepared on a 50 μmol scale. The yield of the product was 19.9 mg, and its estimated purity by LCMS analysis was 100%. Analysis condition A: Retention time=1.6 min; ESI-MS(+) m/z [M+2H]²⁺: 1019.2.

### Preparation of Compound 1170

Compound 1170 was prepared on a 50 μmol scale. The yield of the product was 34.5 mg, and its estimated purity by LCMS analysis was 100%. Analysis condition A: Retention time=1.61 min; ESI-MS(+) m/z [M+2H]²⁺.

### Preparation of Compound 1171

Compound 1171 was prepared on a 50 μmol scale. The yield of the product was 23.5 mg, and its estimated purity by LCMS analysis was 100%. Analysis condition A: Retention time=1.75 min; ESI-MS(+) m/z [M+2H]²⁺: 1019.2.

### Preparation of Compound 1172

Compound 1172 was prepared on a 50 μmol scale. The yield of the product was 8.6 mg, and its estimated purity by LCMS analysis was 92.8%. Analysis condition B: Retention time=1.42 min; ESI-MS(+) m/z [M+2H]²⁺.

### Preparation of Compound 1173

Compound 1173 was prepared on a 50 μmol scale. The yield of the product was 16 mg, and its estimated purity by LCMS analysis was 97.2%. Analysis condition B: Retention time=1.4 min; ESI-MS(+) m/z [M+2H]²⁺: 994.1.

### Preparation of Compound 1174

Compound 1174 was prepared on a 50 μmol scale. The yield of the product was 19.2 mg, and its estimated purity by LCMS analysis was 100%. Analysis condition A: Retention time=1.53 min; ESI-MS(+) m/z [M+2H]²⁺: 1944.3.

### Preparation of Compound 1175

Compound 1175 was prepared on a 50 μmol scale. The yield of the product was 35.3 mg, and its estimated purity by LCMS analysis was 97.1%. Analysis condition B: Retention time=1.67 min; ESI-MS(+) m/z [M+2H]²⁺: 1991.3.

### Preparation of Compound 1176

Compound 1176 was prepared on a 50 μmol scale. The yield of the product was 24.8 mg, and its estimated purity by LCMS analysis was 95%. Analysis condition B: Retention time=1.54 min; ESI-MS(+) m/z [M+2H]²⁺: 655.2.

### Preparation of Compound 1177

Compound 1177 was prepared on a 50 μmol scale. The yield of the product was 21.9 mg, and its estimated purity by LCMS analysis was 98.3%. Analysis condition B: Retention time=1.4 min; ESI-MS(+) m/z [M+2H]²⁺: 966.1.

### Preparation of Compound 1178

Compound 1178 was prepared on a 50 μmol scale. The yield of the product was 33.4 mg, and its estimated purity by LCMS analysis was 99.2%. Analysis condition A: Retention time=1.57 min; ESI-MS(+) m/z [M+2H]²⁺: 975.2.

### Preparation of Compound 1179

Compound 1179 was prepared on a 50 μmol scale. The yield of the product was 10.8 mg, and its estimated purity by LCMS analysis was 100%. Analysis condition A: Retention time=1.42 min; ESI-MS(+) m/z [M+2H]²⁺: 959.

### Preparation of Compound 1180

Compound 1180 was prepared on a 50 μmol scale. The yield of the product was 51 mg, and its estimated purity by LCMS analysis was 97.3%. Analysis condition A: Retention time=1.76 min; ESI-MS(+) m/z [M+2H]²⁺: 1041.2.

### Preparation of Compound 1181

Compound 1181 was prepared on a 50 μmol scale. The yield of the product was 44.7 mg, and its estimated purity by LCMS analysis was 97.1%. Analysis condition B: Retention time=1.57 min; ESI-MS(+) m/z [M+2H]²⁺: 1024.1.

### Preparation of Compound 1182

Compound 1182 was prepared on a 50 μmol scale. The yield of the product was 20.9 mg, and its estimated purity by LCMS analysis was 99%. Analysis condition B: Retention time=1.56 min; ESI-MS(+) m/z [M+2H]²⁺: 1000.

### Preparation of Compound 1183

Compound 1183 was prepared on a 50 μmol scale. The yield of the product was 15.4 mg, and its estimated purity by LCMS analysis was 98.9%. Analysis condition A: Retention time=1.46 min; ESI-MS(+) m/z [M+2H]²⁺: 951.1.

### Preparation of Compound 1184

Compound 1184 was prepared on a 50 μmol scale. The yield of the product was 21.2 mg, and its estimated purity by LCMS analysis was 95.5%. Analysis condition A: Retention time=1.58 min; ESI-MS(+) m/z [M+H]⁺: 1977.1.

### Preparation of Compound 1185

Compound 1185 was prepared on a 50 μmol scale. The yield of the product was 3.3 mg, and its estimated purity by LCMS analysis was 100%. Analysis condition A: Retention time=1.7 min; ESI-MS(+) m/z [M+2H]²⁺: 1020.2.

### Preparation of Compound 1186

Compound 1186 was prepared on a 50 μmol scale. The yield of the product was 32.9 mg, and its estimated purity by LCMS analysis was 97.8%. Analysis condition B: Retention time=1.4 min; ESI-MS(+) m/z [M+2H]²⁺: 950.

### Preparation of Compound 1187

Compound 1187 was prepared on a 50 μmol scale. The yield of the product was 12.5 mg, and its estimated purity by LCMS analysis was 95.7%. Analysis condition B: Retention time=1.56 min; ESI-MS(+) m/z [M+2H]²⁺: 989.5.

### Preparation of Compound 1188

Compound 1188 was prepared on a 50 μmol scale. The yield of the product was 26 mg, and its estimated purity by LCMS analysis was 98.8%. Analysis condition A: Retention time=1.51 min; ESI-MS(+) m/z [M+2H]²⁺: 965.2.

### Preparation of Compound 1189

Compound 1189 was prepared on a 50 μmol scale. The yield of the product was 10.4 mg, and its estimated purity by LCMS analysis was 91.5%. Analysis condition B: Retention time=1.46 min; ESI-MS(+) m/z [M+2H]²⁺: 951.1.

### Preparation of Compound 1190

Compound 1190 was prepared on a 50 μmol scale. The yield of the product was 9.2 mg, and its estimated purity by LCMS analysis was 94%. Analysis condition B: Retention time=1.54 min; ESI-MS(+) m/z [M+2H]²⁺: 948.2.

### Preparation of Compound 1191

Compound 1191 was prepared on a 50 μmol scale. The yield of the product was 18.8 mg, and its estimated purity by LCMS analysis was 98.7%. Analysis condition A: Retention time=1.58 min; ESI-MS(+) m/z [M+H]⁺: 1947.1.

### Preparation of Compound 1192

Compound 1192 was prepared on a 50 μmol scale. The yield of the product was 10.6 mg, and its estimated purity by LCMS analysis was 98.8%. Analysis condition B: Retention time=1.47 min; ESI-MS(+) m/z [M+2H]²⁺: 966.2.

### Preparation of Compound 1193

Compound 1193 was prepared on a 50 μmol scale. The yield of the product was 10 mg, and its estimated purity by LCMS analysis was 100%. Analysis condition A: Retention time=1.61 min; ESI-MS(+) m/z [M+H]⁺: 1951.

### Preparation of Compound 1194

Compound 1194 was prepared on a 50 μmol scale. The yield of the product was 23 mg, and its estimated purity by LCMS analysis was 95.5%. Analysis condition B: Retention time=1.49 min; ESI-MS(+) m/z [M+2H]²⁺: 977.6.

### Preparation of Compound 1195

Compound 1195 was prepared on a 50 μmol scale. The yield of the product was 15.9 mg, and its estimated purity by LCMS analysis was 98.5%. Analysis condition B: Retention time=1.66 min; ESI-MS(+) m/z [M+2H]²⁺: 994.

### Preparation of Compound 1196

Compound 1196 was prepared on a 50 μmol scale. The yield of the product was 11 mg, and its estimated purity by LCMS analysis was 100%. Analysis condition A: Retention time=1.53 min; ESI-MS(+) m/z [M+H]⁺: 1943.

### Preparation of Compound 1197

Compound 1197 was prepared on a 50 μmol scale. The yield of the product was 21.2 mg, and its estimated purity by LCMS analysis was 97.2%. Analysis condition B: Retention time=1.5 min; ESI-MS(+) m/z [M+H]⁺: 1931.

### Preparation of Compound 1198

Compound 1198 was prepared on a 50 μmol scale. The yield of the product was 17.3 mg, and its estimated purity by LCMS analysis was 95.9%. Analysis condition B: Retention time=1.57 min; ESI-MS(+) m/z [M+2H]²⁺: 1985.1.

### Preparation of Compound 1199

Compound 1199 was prepared on a 50 μmol scale. The yield of the product was 18.6 mg, and its estimated purity by LCMS analysis was 94.9%. Analysis condition A: Retention time=1.63 min; ESI-MS(+) m/z [M+H]⁺: 1934.9.

### Preparation of Compound 1200

Compound 1200 was prepared on a 50 μmol scale. The yield of the product was 10.6 mg, and its estimated purity by LCMS analysis was 95.2%. Analysis condition A: Retention time=1.65 min; ESI-MS(+) m/z [M+2H]²⁺: 973.1.

### Preparation of Compound 1201

Compound 1201 was prepared on a 50 μmol scale. The yield of the product was 16.4 mg, and its estimated purity by LCMS analysis was 95.9%. Analysis condition B: Retention time=1.72 min; ESI-MS(+) m/z [M+2H]²⁺: 974.2.

### Preparation of Compound 1202

Compound 1202 was prepared on a 50 μmol scale. The yield of the product was 26.5 mg, and its estimated purity by LCMS analysis was 96.1%. Analysis condition B: Retention time=1.71 min; ESI-MS(+) m/z [M+H]⁺: 1947.

### Preparation of Compound 1203

Compound 1203 was prepared on a 50 μmol scale. The yield of the product was 10.8 mg, and its estimated purity by LCMS analysis was 90.7%. Analysis condition B: Retention time=1.69 min; ESI-MS(+) m/z [M+H]⁺: 1957.9.

### Preparation of Compound 1204

Compound 1204 was prepared on a 50 μmol scale. The yield of the product was 33.4 mg, and its estimated purity by LCMS analysis was 96.7%. Analysis condition B: Retention time=1.69 min; ESI-MS(+) m/z [M+H]⁺: 1919.8.

### Preparation of Compound 1205

Compound 1205 was prepared on a 25 μmol scale. The yield of the product was 3.8 mg, and its estimated purity by LCMS analysis was 98.3%. Analysis condition B: Retention time=1.34 min; ESI-MS(+) m/z [M+2H]²⁺: 973.1.

### Preparation of Compound 1206

Compound 1206 was prepared on a 50 μmol scale. The yield of the product was 15.4 mg, and its estimated purity by LCMS analysis was 97.2%. Analysis condition A: Retention time=1.49 min; ESI-MS(+) m/z [M+H]⁺: 1957.

### Preparation of Compound 1207

Compound 1207 was prepared on a 50 μmol scale. The yield of the product was 2.2 mg, and its estimated purity by LCMS analysis was 92.7%. Analysis condition A: Retention time=1.6 min; ESI-MS(+) m/z [M+2H]²⁺: 958.

### Preparation of Compound 1208

Compound 1208 was prepared on a 50 μmol scale. The yield of the product was 4.2 mg, and its estimated purity by LCMS analysis was 98.3%. Analysis condition B: Retention time=1.62, 1.65 min; ESI-MS(+) m/z [M+H]⁺: 1939.

### Preparation of Compound 1209

Compound 1209 was prepared on a 50 μmol scale. The yield of the product was 2.4 mg, and its estimated purity by LCMS analysis was 93.9%. Analysis condition A: Retention time=1.46 min; ESI-MS(+) m/z [M+2H]²⁺: 972.2.

### Preparation of Compound 1210

Compound 1210 was prepared on a 25 μmol scale. The yield of the product was 5.8 mg, and its estimated purity by LCMS analysis was 100%. Analysis condition A: Retention time=1.05 min; ESI-MS(+) m/z [M+2H]²⁺: 1001.3.

### Preparation of Compound 1211

Compound 1211 was prepared on a 25 μmol scale. The yield of the product was 9.8 mg, and its estimated purity by LCMS analysis was 96.9%. Analysis condition B: Retention time=1.4 min; ESI-MS(+) m/z [M+2H]²⁺: 966.2.

### Preparation of Compound 1212

Compound 1212 was prepared on a 25 μmol scale. The yield of the product was 2.7 mg, and its estimated purity by LCMS analysis was 91.1%. Analysis condition B: Retention time=1.35 min; ESI-MS(+) m/z [M+H]⁺: 1916.2.

### Preparation of Compound 1213

Compound 1213 was prepared on a 25 μmol scale. The yield of the product was 7.2 mg, and its estimated purity by LCMS analysis was 100%. Analysis condition B: Retention time=1.43 min; ESI-MS(+) m/z [M+2H]²⁺: 977.2.

### Preparation of Compound 1214

Compound 1214 was prepared on a 50 μmol scale. The yield of the product was 52.5 mg, and its estimated purity by LCMS analysis was 95.9%. Analysis condition B: Retention time=1.58 min; ESI-MS(+) m/z [M+H]⁺: 1967.8.

### Preparation of Compound 1215

Compound 1215 was prepared on a 50 μmol scale. The yield of the product was 52.2 mg, and its estimated purity by LCMS analysis was 95.9%. Analysis condition A: Retention time=1.56 min; ESI-MS(+) m/z [M+2H]²⁺: 986.1.

### Preparation of Compound 1216

Compound 1216 was prepared on a 50 μmol scale. The yield of the product was 35 mg, and its estimated purity by LCMS analysis was 98%. Analysis condition B: Retention time=1.71 min; ESI-MS(+) m/z [M+H]⁺: 1899.

### Preparation of Compound 1217

Compound 1217 was prepared on a 50 μmol scale. The yield of the product was 12.7 mg, and its estimated purity by LCMS analysis was 98.7%. Analysis condition B: Retention time=1.57 min; ESI-MS(+) m/z [M+H]⁺: 1940.1.

### Preparation of Compound 1218

Compound 1218 was prepared on a 50 μmol scale. The yield of the product was 16.6 mg, and its estimated purity by LCMS analysis was 96.7%. Analysis condition B: Retention time=1.42 min; ESI-MS(+) m/z [M+H]⁺: 1867.9.

### Preparation of Compound 1219

Compound 1219 was prepared on a 50 μmol scale. The yield of the product was 13.8 mg, and its estimated purity by LCMS analysis was 99.3%. Analysis condition A: Retention time=1.48 min; ESI-MS(+) m/z [M+H]⁺: 1867.

### Preparation of Compound 1220

Compound 1220 was prepared on a 25 μmol scale. The yield of the product was 10.1 mg, and its estimated purity by LCMS analysis was 91.2%. Analysis condition A: Retention time=1.45 min; ESI-MS(+) m/z [M+2H]²⁺: 969.1.

### Preparation of Compound 1221

Compound 1221 was prepared on a 25 μmol scale. The yield of the product was 7.9 mg, and its estimated purity by LCMS analysis was 95.7%. Analysis condition A: Retention time=1.48 min; ESI-MS(+) m/z [M+2H]²⁺: 972.1.

### Preparation of Compound 1222

Compound 1222 was prepared on a 25 μmol scale. The yield of the product was 8.3 mg, and its estimated purity by LCMS analysis was 91.6%. Analysis condition A: Retention time=1.51 min; ESI-MS(+) m/z [M+2H]²⁺: 967.1.

### Preparation of Compound 1223

Compound 1223 was prepared on a 25 μmol scale. The yield of the product was 7.1 mg, and its estimated purity by LCMS analysis was 93.7%. Analysis condition A: Retention time=1.45 min; ESI-MS(+) m/z [M+2H]²⁺: 974.2.

### Preparation of Compound 1224

Compound 1224 was prepared on a 25 μmol scale. The yield of the product was 8.1 mg, and its estimated purity by LCMS analysis was 100%. Analysis condition A: Retention time=1.38 min; ESI-MS(+) m/z [M+2H]²⁺: 953.1.

### Preparation of Compound 1225

Compound 1225 was prepared on a 25 μmol scale. The yield of the product was 1.9 mg, and its estimated purity by LCMS analysis was 97.6%. Analysis condition A: Retention time=1.14 min; ESI-MS(+) m/z [M+2H]²⁺: 972.1.

### Preparation of Compound 1226

Compound 1226 was prepared on a 50 μmol scale. The yield of the product was 10.2 mg, and its estimated purity by LCMS analysis was 90.9%. Analysis condition B: Retention time=1.6 min; ESI-MS(+) m/z [M+2H]²⁺: 965.5.

### Preparation of Compound 1227

Compound 1227 was prepared on a 50 μmol scale. The yield of the product was 16.1 mg, and its estimated purity by LCMS analysis was 91.5%. Analysis condition B: Retention time=1.41 min; ESI-MS(+) m/z [M+2H]²⁺: 972.8.

### Preparation of Compound 1228

Compound 1228 was prepared on a 25 μmol scale. The yield of the product was 8.5 mg, and its estimated purity by LCMS analysis was 98.3%. Analysis condition A: Retention time=1.67 min; ESI-MS(+) m/z [M+H]⁺: 1985.2.

### Preparation of Compound 1229

Compound 1229 was prepared on a 25 μmol scale. The yield of the product was 9.1 mg, and its estimated purity by LCMS analysis was 96%. Analysis condition B: Retention time=1.5 min; ESI-MS(+) m/z [M+H]⁺: 1919.

### Preparation of Compound 1230

Compound 1230 was prepared on a 50 μmol scale. The yield of the product was 22.2 mg, and its estimated purity by LCMS analysis was 90.9%. Analysis condition B: Retention time=1.6 min; ESI-MS(+) m/z [M+H]⁺: 1877.

### Preparation of Compound 1231

Compound 1231 was prepared on a 50 μmol scale. The yield of the product was 17 mg, and its estimated purity by LCMS analysis was 97.8%. Analysis condition A: Retention time=1.51 min; ESI-MS(+) m/z [M+H]⁺: 1892.

### Preparation of Compound 1232

Compound 1232 was prepared on a 50 μmol scale. The yield of the product was 11.3 mg, and its estimated purity by LCMS analysis was 96%. Analysis condition A: Retention time=1.43 min; ESI-MS(+) m/z [M+H]⁺: 1914.

### Preparation of Compound 1233

Compound 1233 was prepared on a 50 μmol scale. The yield of the product was 11.1 mg, and its estimated purity by LCMS analysis was 91.2%. Analysis condition B: Retention time=1.51 min; ESI-MS(+) m/z [M+H]⁺: 1945.1.

### Preparation of Compound 1234

Compound 1234 was prepared on a 50 μmol scale. The yield of the product was 4.8 mg, and its estimated purity by LCMS analysis was 94.3%. Analysis condition A: Retention time=1.5 min; ESI-MS(+) m/z [M+H]⁺: 1915.9.

### Preparation of Compound 1235

Compound 1235 was prepared on a 50 μmol scale. The yield of the product was 20.3 mg, and its estimated purity by LCMS analysis was 97%. Analysis condition A: Retention time=1.49 min; ESI-MS(+) m/z [M+H]⁺: 1902.2.

### Preparation of Compound 1236

Compound 1236 was prepared on a 50 μmol scale. The yield of the product was 21.8 mg, and its estimated purity by LCMS analysis was 97.7%. Analysis condition A: Retention time=1.44 min; ESI-MS(+) m/z [M+2H]²⁺: 1001.4.

### Preparation of Compound 1237

Compound 1237 was prepared on a 50 μmol scale. The yield of the product was 17.4 mg, and its estimated purity by LCMS analysis was 92.1%. Analysis condition A: Retention time=1.46 min; ESI-MS(+) m/z [M+H]⁺: 1888.

### Preparation of Compound 1238

Compound 1238 was prepared on a 50 μmol scale. The yield of the product was 18.2 mg, and its estimated purity by LCMS analysis was 98.4%. Analysis condition B: Retention time=1.49 min; ESI-MS(+) m/z [M+H]⁺: 1885.9.

### Preparation of Compound 1239

Compound 1239 was prepared on a 50 μmol scale. The yield of the product was 18.7 mg, and its estimated purity by LCMS analysis was 100%. Analysis condition A: Retention time=1.47 min; ESI-MS(+) m/z [M+H]⁺: 1886.9.

### Preparation of Compound 1240

Compound 1240 was prepared on a 50 μmol scale. The yield of the product was 15.3 mg, and its estimated purity by LCMS analysis was 97%. Analysis condition B: Retention time=1.49 min; ESI-MS(+) m/z [M+H]⁺: 1873.9.

### Preparation of Compound 1241

Compound 1241 was prepared on a 50 μmol scale. The yield of the product was 38.8 mg, and its estimated purity by LCMS analysis was 95%. Analysis condition A: Retention time=1.55 min; ESI-MS(+) m/z [M+H]⁺: 1872.

### Preparation of Compound 1242

Compound 1242 was prepared on a 50 μmol scale. The yield of the product was 15.1 mg, and its estimated purity by LCMS analysis was 100%. Analysis condition A: Retention time=1.41 min; ESI-MS(+) m/z [M+H]⁺: 1913.2.

### Preparation of Compound 1243

Compound 1243 was prepared on a 50 μmol scale. The yield of the product was 24 mg, and its estimated purity by LCMS analysis was 98.9%. Analysis condition A: Retention time=1.54 min; ESI-MS(+) m/z [M+H]⁺: 1857.

### Preparation of Compound 1244

Compound 1244 was prepared on a 50 μmol scale. The yield of the product was 31.9 mg, and its estimated purity by LCMS analysis was 98.7%. Analysis condition A: Retention time=1.52 min; ESI-MS(+) m/z [M+H]⁺: 1942.

### Preparation of Compound 1245

Compound 1245 was prepared on a 50 μmol scale. The yield of the product was 32.5 mg, and its estimated purity by LCMS analysis was 98.7%. Analysis condition A: Retention time=1.63, 1.66 min; ESI-MS(+) m/z [M+H]⁺: 1914.16, 1914.16.

### Preparation of Compound 1246

Compound 1246 was prepared on a 50 μmol scale. The yield of the product was 11.6 mg, and its estimated purity by LCMS analysis was 100%. Analysis condition A: Retention time=1.46 min; ESI-MS(+) m/z [M+H]⁺: 1929.9.

### Preparation of Compound 1247

Compound 1247 was prepared on a 50 μmol scale. The yield of the product was 24.7 mg, and its estimated purity by LCMS analysis was 100%. Analysis condition A: Retention time=1.46 min; ESI-MS(+) m/z [M+H]⁺: 1873.

### Preparation of Compound 1248

Compound 1248 was prepared on a 50 μmol scale. The yield of the product was 16.5 mg, and its estimated purity by LCMS analysis was 100%. Analysis condition A: Retention time=1.41 min; ESI-MS(+) m/z [M+H]⁺: 1859.9.

### Preparation of Compound 1249

Compound 1249 was prepared on a 50 μmol scale. The yield of the product was 30.6 mg, and its estimated purity by LCMS analysis was 100%. Analysis condition A: Retention time=1.51 min; ESI-MS(+) m/z [M+H]⁺: 1887.3.

### Preparation of Compound 1250

Compound 1250 was prepared on a 50 μmol scale. The yield of the product was 29.5 mg, and its estimated purity by LCMS analysis was 83.9%. Analysis condition B: Retention time=1.5 min; ESI-MS(+) m/z [M+H]⁺: 1942.

### Preparation of Compound 1251

Compound 1251 was prepared on a 50 μmol scale. The yield of the product was 22.1 mg, and its estimated purity by LCMS analysis was 96.3%. Analysis condition B: Retention time=1.43 min; ESI-MS(+) m/z [M+H]⁺: 1858.

### Preparation of Compound 1252

Compound 1252 was prepared on a 50 μmol scale. The yield of the product was 33.6 mg, and its estimated purity by LCMS analysis was 97.4%. Analysis condition B: Retention time=1.44 min; ESI-MS(+) m/z [M+H]⁺: 1970.1.

### Preparation of Compound 1253

Compound 1253 was prepared on a 50 μmol scale. The yield of the product was 29.9 mg, and its estimated purity by LCMS analysis was 98.6%. Analysis condition A: Retention time=1.47 min; ESI-MS(+) m/z [M+H]⁺: 1928.1.

### Preparation of Compound 1254

Compound 1254 was prepared on a 50 μmol scale. The yield of the product was 19.8 mg, and its estimated purity by LCMS analysis was 98.9%. Analysis condition A: Retention time=1.47 min; ESI-MS(+) m/z [M+H]⁺: 1929.2.

### Preparation of Compound 1255

Compound 1255 was prepared on a 50 μmol scale. The yield of the product was 30.4 mg, and its estimated purity by LCMS analysis was 100%. Analysis condition B: Retention time=1.58 min; ESI-MS(+) m/z [M+H]⁺: 1873.2.

### Preparation of Compound 1256

Compound 1256 was prepared on a 50 μmol scale. The yield of the product was 31.3 mg, and its estimated purity by LCMS analysis was 94.7%. Analysis condition B: Retention time=1.48 min; ESI-MS(+) m/z [M+3H]³⁺: 639.3.

### Preparation of Compound 1257

Compound 1257 was prepared on a 50 μmol scale. The yield of the product was 2 mg, and its estimated purity by LCMS analysis was 95.5%. Analysis condition B: Retention time=1.37 min; ESI-MS(+) m/z [M+3H]³⁺: 658.1.

### Preparation of Compound 1258

Compound 1258 was prepared on a 50 μmol scale. The yield of the product was 29.3 mg, and its estimated purity by LCMS analysis was 99.3%. Analysis condition A: Retention time=1.49 min; ESI-MS(+) m/z [M+H]⁺: 1843.2.

### Preparation of Compound 1259

Compound 1259 was prepared on a 50 μmol scale. The yield of the product was 36.5 mg, and its estimated purity by LCMS analysis was 96.3%. Analysis condition B: Retention time=1.47 min; ESI-MS(+) m/z [M+H]⁺: 1859.2.

### Preparation of Compound 1260

Compound 1260 was prepared on a 50 μmol scale. The yield of the product was 55.1 mg, and its estimated purity by LCMS analysis was 98.5%. Analysis condition B: Retention time=1.48 min; ESI-MS(+) m/z [M+H]⁺: 1858.9.

### Preparation of Compound 1261

Compound 1261 was prepared on a 50 μmol scale. The yield of the product was 16.6 mg, and its estimated purity by LCMS analysis was 97.7%. Analysis condition B: Retention time=1.46 min; ESI-MS(+) m/z [M+2H]²⁺: 916.4.

### Preparation of Compound 1262

Compound 1262 was prepared on a 50 μmol scale. The yield of the product was 13.9 mg, and its estimated purity by LCMS analysis was 99%. Analysis condition A: Retention time=1.47 min; ESI-MS(+) m/z [M+2H]²⁺: 929.3.

### Preparation of Compound 1263

Compound 1263 was prepare on a 50 μmol scale. The yield of the product was 23.8 mg, and its estimated purity by LCMS analysis was 97.4%. Analysis condition A: Retention time=1.59 min; ESI-MS(+) m/z [M+2H]²⁺: 966.3.

### Preparation of Compound 1264

Compound 1264 was prepared on a 50 μmol scale. The yield of the product was 29.2 mg, and its estimated purity by LCMS analysis was 100%. Analysis condition B: Retention time=1.55 min; ESI-MS(+) m/z [M+2H]²⁺: 910.1.

### Preparation of Compound 1265

Compound 1265 was prepared on a 50 μmol scale. The yield of the product was 40.6 mg, and its estimated purity by LCMS analysis was 95.4%. Analysis condition A: Retention time=1.5 min; ESI-MS(+) m/z [M+2H]²⁺: 920.

### Preparation of Compound 1266

Compound 1266 was prepared on a 50 μmol scale. The yield of the product was 19 mg, and its estimated purity by LCMS analysis was 95.4%. Analysis condition A: Retention time=1.44 min; ESI-MS(+) m/z [M+2H]²⁺: 930.2.

### Preparation of Compound 1267

Compound 1267 was prepared on a 50 μmol scale. The yield of the product was 14.1 mg, and its estimated purity by LCMS analysis was 94.3%. Analysis condition A: Retention time=1.49 min; ESI-MS(+) m/z [M+2H]²⁺: 930.1.

### Preparation of Compound 1268

Compound 1268 was prepared on a 50 μmol scale. The yield of the product was 26.3 mg, and its estimated purity by LCMS analysis was 95.6%. Analysis condition B: Retention time=1.69 min; ESI-MS(+) m/z [M+2H]²⁺: 935.4.

### Preparation of Compound 1269

Compound 1269 was prepared on a 50 μmol scale. The yield of the product was 34.3 mg, and its estimated purity by LCMS analysis was 98.6%. Analysis condition A: Retention time=1.38 min; ESI-MS(+) m/z [M+2H]²⁺: 965.3.

### Preparation of Compound 1270

Compound 1270 was prepared on a 50 μmol scale. The yield of the product was 30.3 mg, and its estimated purity by LCMS analysis was 92%. Analysis condition A: Retention time=1.55 min; ESI-MS(+) m/z [M+H]⁺: 1857.2.

### Preparation of Compound 1271

Compound 1271 was prepared on a 50 μmol scale. The yield of the product was 35.2 mg, and its estimated purity by LCMS analysis was 98.5%. Analysis condition A: Retention time=1.55 min; ESI-MS(+) m/z [M+H]⁺: 1857.2.

### Preparation of Compound 1272

Compound 1272 was prepared on a 50 μmol scale. The yield of the product was 37.2 mg, and its estimated purity by LCMS analysis was 98.2%. Analysis condition B: Retention time=1.59 min; ESI-MS(+) m/z [M+2H]²⁺: 946.3.

### Preparation of Compound 1273

Compound 1273 was prepared on a 50 μmol scale. The yield of the product was 30.7 mg, and its estimated purity by LCMS analysis was 99.1%. Analysis condition A: Retention time=1.5 min; ESI-MS(+) m/z [M+2H]²⁺: 938.3.

### Preparation of Compound 1274

Compound 1274 was prepared on a 50 μmol scale. The yield of the product was 31.9 mg, and its estimated purity by LCMS analysis was 92.2%. Analysis condition B: Retention time=1.6 min; ESI-MS(+) m/z [M+2H]²⁺: 948.

### Preparation of Compound 1275

Compound 1275 was prepared on a 50 μmol scale. The yield of the product was 21.6 mg, and its estimated purity by LCMS analysis was 96.1%. Analysis condition A: Retention time=1.61 min; ESI-MS(+) m/z [M+2H]²⁺: 998.3.

### Preparation of Compound 1276

Compound 1276 was prepared on a 50 μmol scale. The yield of the product was 44.6 mg, and its estimated purity by LCMS analysis was 96.8%. Analysis condition A: Retention time=1.61 min; ESI-MS(+) m/z [M+H]⁺: 1954.

### Preparation of Compound 1277

Compound 1277 was prepared on a 50 μmol scale. The yield of the product was 18.8 mg, and its estimated purity by LCMS analysis was 95.4%. Analysis condition B: Retention time=1.52 min; ESI-MS(+) m/z [M+2H]²⁺: 918.

### Preparation of Compound 1278

Compound 1278 was prepared on a 50 μmol scale. The yield of the product was 19.5 mg, and its estimated purity by LCMS analysis was 98.9%. Analysis condition A: Retention time=1.62 min; ESI-MS(+) m/z [M+2H]²⁺: 950.3.

### Preparation of Compound 1279

Compound 1279 was prepared on a 50 μmol scale. The yield of the product was 34.5 mg, and its estimated purity by LCMS analysis was 95.6%. Analysis condition B: Retention time=1.54 min; ESI-MS(+) m/z [M+H]⁺: 1855.2.

### Preparation of Compound 1280

Compound 1280 was prepared on a 50 μmol scale. The yield of the product was 9.4 mg, and its estimated purity by LCMS analysis was 88.8%. Analysis condition B: Retention time=1.45 min; ESI-MS(+) m/z [M+2H]²⁺: 965.4.

### Preparation of Compound 1281

Compound 1281 was prepared on a 50 μmol scale. The yield of the product was 26.9 mg, and its estimated purity by LCMS analysis was 97.1%. Analysis condition B: Retention time=1.59 min; ESI-MS(+) m/z [M+2H]²⁺: 901.7.

### Preparation of Compound 1282

Compound 1282 was prepared on a 50 μmol scale. The yield of the product was 14 mg, and its estimated purity by LCMS analysis was 100%. Analysis condition A: Retention time=1.47 min; ESI-MS(+) m/z [M+2H]²⁺: 979.2.

### Preparation of Compound 1283

Compound 1283 was prepared on a 50 μmol scale. The yield of the product was 7.3 mg, and its estimated purity by LCMS analysis was 97.6%. Analysis condition A: Retention time=1.48 min; ESI-MS(+) m/z [M+2H]²⁺: 951.

### Preparation of Compound 1284

Compound 1284 was prepared on a 50 μmol scale. The yield of the product was 24.5 mg, and its estimated purity by LCMS analysis was 94.7%. Analysis condition B: Retention time=1.52 min; ESI-MS(+) m/z [M+2H]²⁺: 971.

### Preparation of Compound 1285

Compound 1285 was prepared on a 50 μmol scale. The yield of the product was 13.5 mg, and its estimated purity by LCMS analysis was 96%. Analysis condition B: Retention time=1.52 min; ESI-MS(+) m/z [M+2H]²⁺ 979.4.

### Preparation of Compound 1286

Compound 1286 was prepared on a 50 μmol scale. The yield of the product was 5.9 mg, and its estimated purity by LCMS analysis was 91.1%. Analysis condition B: Retention time=1.5 min; ESI-MS(+) m/z [M+2H]²⁺: 951.6.

### Preparation of Compound 1287

Compound 1287 was prepared on a 50 μmol scale. The yield of the product was 8.2 mg, and its estimated purity by LCMS analysis was 86.1%. Analysis condition A: Retention time=1.61, 1.64 min; ESI-MS(+) m/z [M+2H]²⁺: 971.

### Preparation of Compound 1288

Compound 1288 was prepared on a 50 μmol scale. The yield of the product was 26.2 mg, and its estimated purity by LCMS analysis was 92.1%. Analysis condition A: Retention time=1.45 min; ESI-MS(+) m/z [M+2H]²⁺: 958.2.

### Preparation of Compound 1289

Compound 1289 was prepared on a 50 μmol scale. The yield of the product was 23.3 mg, and its estimated purity by LCMS analysis was 93.2%. Analysis condition A: Retention time=1.53 min; ESI-MS(+) m/z [M+2H]²⁺: 958.2.

### Preparation of Compound 1290

Compound 1290 was prepared on a 50 μmol scale. The yield of the product was 29.8 mg, and its estimated purity by LCMS analysis was 98.6%. Analysis condition B: Retention time=1.66 min; ESI-MS(+) m/z [M+2H]²⁺: 936.4.

### Preparation of Compound 1291

Compound 1291 was prepared on a 50 μmol scale. The yield of the product was 27 mg, and its estimated purity by LCMS analysis was 93.3%. Analysis condition B: Retention time=1.69 min; ESI-MS(+) m/z [M+2H]²⁺: 965.1.

### Preparation of Compound 1292

Compound 1292 was prepared on a 50 μmol scale. The yield of the product was 14.5 mg, and its estimated purity by LCMS analysis was 96.4%. Analysis condition A: Retention time=1.49 min; ESI-MS(+) m/z [M+2H]²⁺: 946.2.

### Preparation of Compound 1293

Compound 1293 was prepared on a 50 μmol scale. The yield of the product was 19.8 mg, and its estimated purity by LCMS analysis was 98.1%. Analysis condition B: Retention time=1.59, 1.62 min; ESI-MS(+) m/z [M+H]⁺: 1899.3.

### Preparation of Compound 1294

Compound 1294 was prepare on a 50 μmol scale. The yield of the product was 26 mg, and its estimated purity by LCMS analysis was 93.4%. Analysis condition B: Retention time=1.67 min; ESI-MS(+) m/z [M+2H]²⁺: 970.4.

### Preparation of Compound 1295

Compound 1295 was prepared on a 50 μmol scale. The yield of the product was 19.2 mg, and its estimated purity by LCMS analysis was 92%. Analysis condition B: Retention time=1.56 min; ESI-MS(+) m/z [M+2H]²⁺: 922.1.

### Preparation of Compound 1296

Compound 1296 was prepared on a 50 μmol scale. The yield of the product was 26.3 mg, and its estimated purity by LCMS analysis was 96.4%. Analysis condition B: Retention time=1.65 min; ESI-MS(+) m/z [M+2H]²⁺: 942.4.

### Preparation of Compound 1297

Compound 1297 was prepared on a 50 μmol scale. The yield of the product was 32.6 mg, and its estimated purity by LCMS analysis was 86.4%. Analysis condition A: Retention time=1.73, 1.75 min: ESI-MS(+) m/z [M+2H]²⁺: 921.

### Preparation of Compound 1298

Compound 1298 was prepared on a 50 μmol scale. The yield of the product was 19.7 mg, and its estimated purity by LCMS analysis was 96.6%. Analysis condition A: Retention time=1.85 min; ESI-MS(+) m/z [M+2H]²⁺: 935.1.

### Preparation of Compound 1299

Compound 1299 was prepared on a 50 μmol scale. The yield of the product was 29.3 mg, and its estimated purity by LCMS analysis was 98.2%. Analysis condition B: Retention time=1.79 min; ESI-MS(+) m/z [M+H]⁺: 1871.

### Preparation of Compound 1300

Compound 1300 was prepared on a 50 μmol scale. The yield of the product was 20.8 mg, and its estimated purity by LCMS analysis was 100%. Analysis condition A: Retention time=1.81 min; ESI-MS(+) m/z [M+H]⁺: 1844.5.

### Preparation of Compound 1301

Compound 1301 was prepared on a 50 μmol scale. The yield of the product was 39.7 mg, and its estimated purity by LCMS analysis was 98.4%. Analysis condition A: Retention time=1.75 min; ESI-MS(+) m/z [M+H]⁺: 1873.

### Preparation of Compound 1302

Compound 1302 was prepared on a 50 μmol scale. The yield of the product was 15.9 mg, and its estimated purity by LCMS analysis was 98.9%. Analysis condition B: Retention time=1.72 min; ESI-MS(+) m/z [M+H]⁺: 1881.2.

### Preparation of Compound 1303

Compound 1303 was prepared on a 50 μmol scale. The yield of the product was 24.7 mg, and its estimated purity by LCMS analysis was 95.4%. Analysis condition B: Retention time=1.78 min; ESI-MS(+) m/z [M+2H]²⁺: 954.2.

### Preparation of Compound 1304

Compound 1304 was prepared on a 50 μmol scale. The yield of the product was 10.3 mg, and its estimated purity by LCMS analysis was 99%. Analysis condition A: Retention time=1.9 min; ESI-MS(+) m/z [M+H]⁺: 1997.

### Preparation of Compound 1305

Compound 1305 was prepared on a 50 μmol scale. The yield of the product was 8.4 mg, and its estimated purity by LCMS analysis was 98%. Analysis condition B: Retention time=1.79 min; ESI-MS(+) m/z [M+H]⁺: 1899.

### Preparation of Compound 1306

Compound 1306 was prepared on a 50 μmol scale. The yield of the product was 8.6 mg, and its estimated purity by LCMS analysis was 100%. Analysis condition A: Retention time=1.63 min; ESI-MS(+) m/z [M+2H]²⁺: 955.4.

### Preparation of Compound 1307

Compound 1307 was prepared on a 50 μmol scale. The yield of the product was 14.3 mg, and its estimated purity by LCMS analysis was 96%. Analysis condition A: Retention time=1.88 min; ESI-MS(+) m/z [M+2H]²⁺: 957.7.

### Preparation of Compound 1308

Compound 1308 was prepared on a 50 μmol scale. The yield of the product was 12 mg, and its estimated purity by LCMS analysis was 87.2%. Analysis condition B: Retention time=1.78, 1.81 min; ESI-MS(+) m/z [M+H]⁺: 1909.3.

### Preparation of Compound 1309

Compound 1309 was prepared on a 50 μmol scale. The yield of the product was 13.2 mg, and its estimated purity by LCMS analysis was 100%. Analysis condition A: Retention time=1.85 min; ESI-MS(+) m/z [M+H]⁺: 1902.8.

### Preparation of Compound 1310

Compound 1310 was prepared on a 50 μmol scale. The yield of the product was 6.3 mg, and its estimated purity by LCMS analysis was 97.1%. Analysis condition B: Retention time=1.71 min; ESI-MS(+) m/z [M+2H]²⁺: 960.2.

### Preparation of Compound 1311

Compound 1311 was prepared on a 50 μmol scale. The yield of the product was 9 mg, and its estimated purity by LCMS analysis was 94.7%. Analysis condition B: Retention time=1.72 min; ESI-MS(+) m/z [M+H]⁺: 1898.7.

### Preparation of Compound 1312

Compound 1312 was prepared on a 50 μmol scale. The yield of the product was 8.4 mg, and its estimated purity by LCMS analysis was 94.4%. Analysis condition B: Retention time=1.85 min; ESI-MS(+) m/z [M+H]⁺: 1910.

### Preparation of Compound 1313

Compound 1313 was prepared on a 50 μmol scale. The yield of the product was 4.9 mg, and its estimated purity by LCMS analysis was 93.9%. Analysis condition B: Retention time=1.87, 1.92 min; ESI-MS(+) m/z [M+H]⁺: 1915.3.

### Preparation of Compound 1314

Compound 1314 was prepared on a 50 μmol scale. The yield of the product was 6.7 mg, and its estimated purity by LCMS analysis was 94.2%. Analysis condition B: Retention time=1.66 min; ESI-MS(+) m/z [M+H]⁺: 1910.7.

### Preparation of Compound 1315

Compound 1315 was prepared on a 50 μmol scale. The yield of the product was 7.4 mg, and its estimated purity by LCMS analysis was 100%. Analysis condition A: Retention time=1.83, 1.95 min; ESI-MS(+) m/z [M+H]⁺: 1903.3.

### Preparation of Compound 1316

Compound 1316 was prepared on a 50 μmol scale. The yield of the product was 11.9 mg, and its estimated purity by LCMS analysis was 98.6%. Analysis condition A: Retention time=1.59 min; ESI-MS(+) m/z [M+H]⁺: 1918.1.

### Preparation of Compound 1317

Compound 1317 was prepared on a 50 μmol scale. The yield of the product was 5.9 mg, and its estimated purity by LCMS analysis was 100%. Analysis condition A: Retention time=1.32 min; ESI-MS(+) m/z [M+H]⁺: 1929.

### Preparation of Compound 1318

Compound 1318 was prepared on a 50 μmol scale. The yield of the product was 4 mg, and its estimated purity by LCMS analysis was 90%. Analysis condition B: Retention time=1.38, 1.46 min; ESI-MS(+) m/z [M+2H]²⁺: 944.

### Preparation of Compound 1319

Compound 1319 was prepared on a 50 μmol scale. The yield of the product was 6 mg, and its estimated purity by LCMS analysis was 94.8%. Analysis condition A: Retention time=1.55, 1.6 min; ESI-MS(+) m/z [M+2H]²⁺: 973.55, 973.26.

### Preparation of Compound 1320

Compound 1320 was prepared on a 50 μmol scale. The yield of the product was 7.6 mg, and its estimated purity by LCMS analysis was 97%. Analysis condition B: Retention time=1.38 min; ESI-MS(+) m/z [M+H]⁺: 1886.

### Preparation of Compound 1321

Compound 1321 was prepared on a 50 μmol scale. The yield of the product was 9.7 mg, and its estimated purity by LCMS analysis was 98.1%. Analysis condition B: Retention time=1.46 min; ESI-MS(+) m/z [M+H]⁺: 1958.9.

### Preparation of Compound 1322

Compound 1322 was prepared on a 50 μmol scale. The yield of the product was 11.7 mg, and its estimated purity by LCMS analysis was 95.8%. Analysis condition A: Retention time=1.72 min; ESI-MS(+) m/z [M+H]⁺: 1914.1.

### Preparation of Compound 1323

Compound 1323 was prepared on a 50 μmol scale. The yield of the product was 14.7 mg, and its estimated purity by LCMS analysis was 93.9%. Analysis condition B: Retention time=1.39 min; ESI-MS(+) m/z [M+H]⁺: 1935.1.

### Preparation of Compound 1324

Compound 1324 was prepared on a 50 μmol scale. The yield of the product was 6.9 mg, and its estimated purity by LCMS analysis was 97.4%. Analysis condition B: Retention time=1.51 min; ESI-MS(+) m/z [M+2H]²⁺: 1005.1.

### Preparation of Compound 1325

Compound 1325 was prepared on a 50 μmol scale. The yield of the product was 7.6 mg, and its estimated purity by LCMS analysis was 95.5%. Analysis condition B: Retention time=1.41 min; ESI-MS(+) m/z [M+2H]²⁺: 968.2.

### Preparation of Compound 1326

Compound 1326 was prepared on a 50 μmol scale. The yield of the product was 6.2 mg, and its estimated purity by LCMS analysis was 98.2%. Analysis condition B: Retention time=1.5 min; ESI-MS(+) m/z [M+H]⁺: 1977.9.

### Preparation of Compound 1327

Compound 1327 was prepared on a 50 μmol scale. The yield of the product was 1.4 mg, and its estimated purity by LCMS analysis was 100%. Analysis condition A: Retention time=1.44 min; ESI-MS(+) m/z [M+H]⁺: 1934.9.

### Preparation of Compound 1328

Compound 1328 was prepared on a 50 μmol scale. The yield of the product was 6.4 mg, and its estimated purity by LCMS analysis was 100%. Analysis condition A: Retention time=1.33 min; ESI-MS(+) m/z [M+2]⁺: 1963.9.

### Preparation of Compound 1329

Compound 1329 was prepared on a 50 μmol scale. The yield of the product was 3 mg, and its estimated purity by LCMS analysis was 84.6%. Analysis condition B: Retention time=1.47 min; ESI-MS(+) m/z [M+H]⁺: 1991.9.

### Preparation of Compound 1330

Compound 1330 was prepared on a 50 μmol scale. The yield of the product was 9.3 mg, and its estimated purity by LCMS analysis was 91.7%. Analysis condition A: Retention time=1.36, 1.41 min; ESI-MS(+) m/z [M+H]⁺: 1963.4.

### Preparation of Compound 1331

Compound 1331 was prepared on a 50 μmol scale. The yield of the product was 1.2 mg, and its estimated purity by LCMS analysis was 96.2%. Analysis condition B: Retention time=1.41 min; ESI-MS(+) m/z [M+H]⁺: 1992.9.

### Preparation of Compound 1332

Compound 1332 was prepared on a 50 μmol scale. The yield of the product was 5.1 mg, and its estimated purity by LCMS analysis was 91.9%. Analysis condition A: Retention time=1.33 min; ESI-MS(+) m/z [M+H]⁺: 1962.9.

### Preparation of Compound 1333

Compound 1333 was prepared on a 50 μmol scale. The yield of the product was 9.1 mg, and its estimated purity by LCMS analysis was 92.1%. Analysis condition B: Retention time=1.34 min; ESI-MS(+) m/z [M+H]⁺: 1919.9.

### Preparation of Compound 1334

Compound 1334 was prepared on a 50 μmol scale. The yield of the product was 8.6 mg, and its estimated purity by LCMS analysis was 87.1%. Analysis condition B: Retention time=1.36 min; ESI-MS(+) m/z [M+H]⁺: 1919.8.

### Preparation of Compound 1335

Compound 1335 was prepared on a 50 μmol scale. The yield of the product was 9.3 mg, and its estimated purity by LCMS analysis was 96.8%. Analysis condition B: Retention time=1.45 min; ESI-MS(+) m/z [M+3H]³⁺: 665.2.

### Preparation of Compound 1336

Compound 1336 was prepared on a 50 μmol scale. The yield of the product was 2.8 mg, and its estimated purity by LCMS analysis was 93.5%. Analysis condition A: Retention time=1.47 min; ESI-MS(+) m/z [M+H]⁺: 1980.2.

### Preparation of Compound 1337

Compound 1337 was prepared on a 50 μmol scale. The yield of the product was 0.6 mg, and its estimated purity by LCMS analysis was 100%. Analysis condition B: Retention time=1.81 min; ESI-MS(+) m/z [M+2H]²⁺: 990.

### Preparation of Compound 1338

Compound 1338 was prepared on a 50 μmol scale. The yield of the product was 3.7 mg, and its estimated purity by LCMS analysis was 100%. Analysis condition A: Retention time=1.53 min; ESI-MS(+) m/z [M+2H]²⁺ 991.1.

### Preparation of Compound 1339

Compound 1339 was prepared on a 50 μmol scale. The yield of the product was 9.2 mg, and its estimated purity by LCMS analysis was 86%. Analysis condition A: Retention time=1.44 min; ESI-MS(+) m/z [M+2H]²⁺: 961.

### Preparation of Compound 1340

Compound 1340 was prepared on a 50 μmol scale. The yield of the product was 4.7 mg, and its estimated purity by LCMS analysis was 100%. Analysis condition A: Retention time=1.54 min; ESI-MS(+) m/z [M+H]⁺: 1887.

### Preparation of Compound 1341

Compound 1341 was prepared on a 50 μmol scale. The yield of the product was 21.1 mg, and its estimated purity by LCMS analysis was 100%. Analysis condition A: Retention time=1.53 min; ESI-MS(+) m/z [M+H]⁺: 1900.2.

### Preparation of Compound 1342

Compound 1342 was prepared on a 50 μmol scale. The yield of the product was 8.4 mg, and its estimated purity by LCMS analysis was 100%. Analysis condition A: Retention time=1.55 min; ESI-MS(+) m/z [M+H]⁺: 1932.

### Preparation of Compound 1343

Compound 1343 was prepared on a 50 μmol scale. The yield of the product was 15.7 mg, and its estimated purity by LCMS analysis was 98.7%. Analysis condition B: Retention time=1.41 min; ESI-MS(+) m/z [M+H]⁺: 1942.

### Preparation of Compound 1344

Compound 1344 was prepared on a 50 μmol scale. The yield of the product was 7.8 mg, and its estimated purity by LCMS analysis was 98.8%. Analysis condition B: Retention time=1.45 min; ESI-MS(+) m/z [M+H]⁺: 1955.9.

### Preparation of Compound 1345

Compound 1345 was prepared on a 50 μmol scale. The yield of the product was 18 mg, and its estimated purity by LCMS analysis was 96%. Analysis condition A: Retention time=1.55 min; ESI-MS(+) m/z [M+H]⁺: 1872.9.

### Preparation of Compound 1346

Compound 1346 was prepared on a 50 μmol scale. The yield of the product was 17.4 mg, and its estimated purity by LCMS analysis was 95.6%. Analysis condition B: Retention time=1.48 min; ESI-MS(+) m/z [M+H]⁺: 1887.

### Preparation of Compound 1347

Compound 1347 was prepared on a 50 μmol scale. The yield of the product was 13 mg, and its estimated purity by LCMS analysis was 95.4%. Analysis condition A: Retention time=1.56 min; ESI-MS(+) m/z [M+H]⁺: 1902.

### Preparation of Compound 1348

Compound 1348 was prepared on a 50 μmol scale. The yield of the product was 20.4 mg, and its estimated purity by LCMS analysis was 100%. Analysis condition A: Retention time=1.54 min; ESI-MS(+) m/z [M+H]⁺: 1916.2.

### Preparation of Compound 1349

Compound 1349 was prepared on a 50 μmol scale. The yield of the product was 4.7 mg, and its estimated purity by LCMS analysis was 96.8%. Analysis condition B: Retention time=1.51 min; ESI-MS(+) m/z [M+2H]²⁺: 979.9.

### Preparation of Compound 1350

Compound 1350 was prepared on a 50 μmol scale. The yield of the product was 6.2 mg, and its estimated purity by LCMS analysis was 96%. Analysis condition A: Retention time=1.74 min; ESI-MS(+) m/z [M+H]⁺: 1871.6.

### Preparation of Compound 1351

Compound 1351 was prepared on a 50 μmol scale. The yield of the product was 9.5 mg, and its estimated purity by LCMS analysis was 98.8%. Analysis condition B: Retention time=1.58 min; ESI-MS(+) m/z [M+3H]³⁺: 648.3.

### Preparation of Compound 1352

Compound 1352 was prepare on a 50 μmol scale. The yield of the product was 10.6 mg, and its estimated purity by LCMS analysis was 94.2%. Analysis condition A: Retention time=1.7 min; ESI-MS(+) m/z [M+H]⁺: 1899.1.

### Preparation of Compound 1353

Compound 1353 was prepared on a 50 μmol scale. The yield of the product was 15.6 mg, and its estimated purity by LCMS analysis was 98.9%. Analysis condition A: Retention time=1.54 min; ESI-MS(+) m/z [M+2H]²⁺: 950.2.

### Preparation of Compound 1354

Compound 1354 was prepared on a 50 μmol scale. The yield of the product was 1.8 mg, and its estimated purity by LCMS analysis was 93.6%. Analysis condition B: Retention time=1.35 min; ESI-MS(+) m/z [M+2H]²⁺: 957.2.

### Preparation of Compound 1355

Compound 1355 was prepared on a 50 μmol scale. The yield of the product was 7 mg, and its estimated purity by LCMS analysis was 97.7%. Analysis condition A: Retention time=1.4 min; ESI-MS(+) m/z [M+2H]²⁺: 951.5.

### Preparation of Compound 1356

Compound 1356 was prepared on a 50 μmol scale. The yield of the product was 17.9 mg, and its estimated purity by LCMS analysis was 93.4%. Analysis condition B: Retention time=1.54 min; ESI-MS(+) m/z [M+H]⁺: 1924.1.

### Preparation of Compound 1357

Compound 1357 was prepared on a 50 μmol scale. The yield of the product was 18.7 mg, and its estimated purity by LCMS analysis was 94.3%. Analysis condition B: Retention time=1.54, 1.58 min; ESI-MS(+) m/z [M+H]⁺: 1915.3.

### Preparation of Compound 1358

Compound 1358 was prepared on a 50 μmol scale. The yield of the product was 16.7 mg, and its estimated purity by LCMS analysis was 100%. Analysis condition A: Retention time=1.6 min; ESI-MS(+) m/z [M+H]⁺: 1867.1.

### Preparation of Compound 1359

Compound 1359 was prepared on a 50 μmol scale. The yield of the product was 22.4 mg, and its estimated purity by LCMS analysis was 98%. Analysis condition B: Retention time=1.6 min; ESI-MS(+) m/z [M+H]⁺: 1856.9.

### Preparation of Compound 1360

Compound 1360 was prepared on a 50 μmol scale. The yield of the product was 26.6 mg, and its estimated purity by LCMS analysis was 94%. Analysis condition B: Retention time=1.53 min; ESI-MS(+) m/z [M+H]⁺: 1815.

### Preparation of Compound 1361

Compound 1361 was prepared on a 50 μmol scale. The yield of the product was 8.2 mg, and its estimated purity by LCMS analysis was 100%. Analysis condition A: Retention time=1.6 min; ESI-MS(+) m/z [M+H]⁺: 1830.1.

### Preparation of Compound 1362

Compound 1362 was prepared on a 50 μmol scale. The yield of the product was 22 mg, and its estimated purity by LCMS analysis was 97.8%. Analysis condition B: Retention time=1.41, 1.46 min; ESI-MS(+) m/z [M+H]⁺: 1881.89, 1880.98.

### Preparation of Compound 1363

Compound 1363 was prepared on a 50 μmol scale. The yield of the product was 26.3 mg, and its estimated purity by LCMS analysis was 96.3%. Analysis condition A: Retention time=1.66 min; ESI-MS(+) m/z [M+H]⁺: 1872.6.

### Preparation of Compound 1364

Compound 1364 was prepared on a 50 μmol scale. The yield of the product was 1.5 mg, and its estimated purity by LCMS analysis was 90.2%. Analysis condition B: Retention time=1.68 min; ESI-MS(+) m/z [M+2H]²⁺: 968.3.

### Preparation of Compound 1365

Compound 1365 was prepared on a 50 μmol scale. The yield of the product was 6.6 mg, and its estimated purity by LCMS analysis was 100%. Analysis condition A: Retention time=1.86 min; ESI-MS(+) m/z [M+H]⁺: 1885.2.

### Preparation of Compound 1366

Compound 1366 was prepared on a 50 μmol scale. The yield of the product was 2.4 mg, and its estimated purity by LCMS analysis was 96.1%. Analysis condition B: Retention time=1.79 min; ESI-MS(+) m/z [M+H]⁺: 1904.8.

### Preparation of Compound 1367

Compound 1367 was prepared on a 50 μmol scale. The yield of the product was 5.4 mg, and its estimated purity by LCMS analysis was 91.1%. Analysis condition B: Retention time=1.82 min; ESI-MS(+) m/z [M+H]⁺: 1872.2.

### Preparation of Compound 1368

Compound 1368 was prepared on a 50 μmol scale. The yield of the product was 1.5 mg, and its estimated purity by LCMS analysis was 87.2%. Analysis condition B: Retention time=1.88 min; ESI-MS(+) m/z [M+H]⁺: 1958.

### Preparation of Compound 1369

Compound 1369 was prepared on a 50 μmol scale. The yield of the product was 11.5 mg, and its estimated purity by LCMS analysis was 99.3%. Analysis condition B: Retention time=1.8 min; ESI-MS(+) m/z [M+H]⁺: 1872.2.

### Preparation of Compound 1370

Compound 1370 was prepared on a 500 μmol scale. The yield of the product was 5.6 mg, and its estimated purity by LCMS analysis was 100%. Analysis condition A: Retention time=1.62 min; ESI-MS(+) m/z [M+H]⁺: 1860.2.

### Preparation of Compound 1371

Compound 1371 was prepared on a 50 μmol scale. The yield of the product was 7 mg, and its estimated purity by LCMS analysis was 86.4%. Analysis condition B: Retention time=1.75 min; ESI-MS(+) m/z [M+H]⁺: 1885.

### Preparation of Compound 1372

Compound 1372 was prepared on a 50 μmol scale. The yield of the product was 21.2 mg, and its estimated purity by LCMS analysis was 94.7%. Analysis condition B: Retention time=1.56 min; ESI-MS(+) m/z [M+H]⁺: 1958.

### Preparation of Compound 1373

Compound 1373 was prepared on a 50 μmol scale. The yield of the product was 5.9 mg, and its estimated purity by LCMS analysis was 100%. Analysis condition A: Retention time=1.67 min; ESI-MS(+) m/z [M+H]⁺: 1870.2.

### Preparation of Compound 1374

Compound 1374 was prepared on a 50 μmol scale. The yield of the product was 13.7 mg, and its estimated purity by LCMS analysis was 97%. Analysis condition B: Retention time=1.46 min; ESI-MS(+) m/z [M+H]⁺: 1914.8.

### Preparation of Compound 1375

Compound 1375 was prepared on a 50 μmol scale. The yield of the product was 17.7 mg, and its estimated purity by LCMS analysis was 96.4%. Analysis condition B: Retention time=1.61 min; ESI-MS(+) m/z [M+H]⁺: 1890.9.

### Preparation of Compound 1376

Compound 1376 was prepared on a 50 μmol scale. The yield of the product was 22 mg, and its estimated purity by LCMS analysis was 97.6%. Analysis condition B: Retention time=1.59 min; ESI-MS(+) m/z [M+H]⁺: 1856.8.

### Preparation of Compound 1377

Compound 1377 was prepared on a 50 μmol scale. The yield of the product was 22.7 mg, and its estimated purity by LCMS analysis was 98.2%. Analysis condition B: Retention time=1.66 min; ESI-MS(+) m/z [M+H]⁺: 1901.8.

### Preparation of Compound 1378

Compound 1378 was prepared on a 50 μmol scale. The yield of the product was 25.7 mg, and its estimated purity by LCMS analysis was 97.8%. Analysis condition B: Retention time=1.64 min; ESI-MS(+) m/z [M+H]⁺: 1871.1.

### Preparation of Compound 1379

Compound 1379 was prepared on a 50 μmol scale. The yield of the product was 13.2 mg, and its estimated purity by LCMS analysis was 100%. Analysis condition A: Retention time=1.67 min; ESI-MS(+) m/z [M+H]⁺: 1871.2.

### Preparation of Compound 1380

Compound 1380 was prepared on a 50 μmol scale. The yield of the product was 18 mg, and its estimated purity by LCMS analysis was 100%. Analysis condition A: Retention time=1.67 min: ESI-MS(+) m/z [M+H]⁺: 1852.9.

### Preparation of Compound 1381

Compound 1381 was prepared on a 50 μmol scale. The yield of the product was 7.1 mg, and its estimated purity by LCMS analysis was 90.2%. Analysis condition A: Retention time=1.89 min; ESI-MS(+) m/z [M+H]⁺: 1908.2.

### Preparation of Compound 1382

Compound 1382 was prepared on a 50 μmol scale. The yield of the product was 7.7 mg, and its estimated purity by LCMS analysis was 98.8%. Analysis condition A: Retention time=1.85 min: ESI-MS(+) m/z [M+H]⁺: 1928.3.

### Preparation of Compound 1383

Compound 1383 was prepared on a 50 μmol scale. The yield of the product was 16.7 mg, and its estimated purity by LCMS analysis was 91.7%. Analysis condition A: Retention time=1.57 min; ESI-MS(+) m/z [M+2H]²⁺: 955.1.

### Preparation of Compound 1384

Compound 1384 was prepared on a 50 μmol scale. The yield of the product was 1.9 mg, and its estimated purity by LCMS analysis was 94.1%. Analysis condition A: Retention time=1.68 min; ESI-MS(+) m/z [M+H]⁺: 1843.2.

### Preparation of Compound 1385

Compound 1385 was prepared on a 50 μmol scale. The yield of the product was 14.1 mg, and its estimated purity by LCMS analysis was 97.6%. Analysis condition B: Retention time=1.8 min; ESI-MS(+) m/z [M+H]⁺: 1935.3.

### Preparation of Compound 1386

Compound 1386 was prepared on a 50 μmol scale. The yield of the product was 26.2 mg, and its estimated purity by LCMS analysis was 97.9%. Analysis condition B: Retention time=1.53 min; ESI-MS(+) m/z [M+H]⁺: 1886.2.

### Preparation of Compound 1387

Compound 1387 was prepared on a 50 μmol scale. The yield of the product was 23.4 mg, and its estimated purity by LCMS analysis was 95.9%. Analysis condition B: Retention time=1.55 min; ESI-MS(+) m/z [M+H]⁺: 1917.2.

### Preparation of Compound 1388

Compound 1388 was prepared on a 50 μmol scale. The yield of the product was 27.9 mg, and its estimated purity by LCMS analysis was 100%. Analysis condition A: Retention time=1.57 min; ESI-MS(+) m/z [M+H]⁺: 1940.2.

### Preparation of Compound 1389

Compound 1389 was prepared on a 50 μmol scale. The yield of the product was 37.2 mg, and its estimated purity by LCMS analysis was 95.4%. Analysis condition B: Retention time=1.46 min; ESI-MS(+) m/z [M+2H]²⁺: 951.1.

### Preparation of Compound 1390

Compound 1390 was prepared on a 50 μmol scale. The yield of the product was 35.8 mg, and its estimated purity by LCMS analysis was 100%. Analysis condition A: Retention time=1.5 min; ESI-MS(+) m/z [M+H]⁺: 1932.2.

### Preparation of Compound 1391

Compound 1391 was prepared on a 50 μmol scale. The yield of the product was 18.9 mg, and its estimated purity by LCMS analysis was 100%. Analysis condition A: Retention time=1.49 min; ESI-MS(+) m/z [M+2H]²⁺: 936.1.

### Preparation of Compound 1392

Compound 1392 was prepared on a 50 μmol scale. The yield of the product was 13.4 mg, and its estimated purity by LCMS analysis was 98.9%. Analysis condition B: Retention time=1.81 min; ESI-MS(+) m/z [M+H]⁺: 1856.3.

### Preparation of Compound 1393

Compound 1393 was prepared on a 50 μmol scale. The yield of the product was 18.3 mg, and its estimated purity by LCMS analysis was 98.8%. Analysis condition B: Retention time=1.82 min; ESI-MS(+) m/z [M+H]⁺: 1891.2.

### Preparation of Compound 1394

Compound 1394 was prepared on a 50 μmol scale. The yield of the product was 31.5 mg, and its estimated purity by LCMS analysis was 98.1%. Analysis condition B: Retention time=1.35 min; ESI-MS(+) m/z [M+2H]²⁺: 943.1.

### Preparation of Compound 1395

Compound 1395 was prepared on a 50 μmol scale. The yield of the product was 24 mg, and its estimated purity by LCMS analysis was 97.6%. Analysis condition B: Retention time=1.45 min; ESI-MS(+) m/z [M+2H]²⁺: 942.9.

### Preparation of Compound 1396

Compound 1396 was prepared on a 50 μmol scale. The yield of the product was 9.3 mg, and its estimated purity by LCMS analysis was 97.6%. Analysis condition B: Retention time=1.65 min; ESI-MS(+) m/z [M+H]⁺: 1916.1.

### Preparation of Compound 1397

Compound 1397 was prepared on a 50 μmol scale. The yield of the product was 11.6 mg, and its estimated purity by LCMS analysis was 97.8%. Analysis condition B: Retention time=1.73 min; ESI-MS(+) m/z [M+H]⁺: 1866.2.

### Preparation of Compound 1398

Compound 1398 was prepared on a 50 μmol scale. The yield of the product was 17.4 mg, and its estimated purity by LCMS analysis was 97.3%. Analysis condition A: Retention time=1.51 min; ESI-MS(+) m/z [M+2H]²⁺: 917.2.

### Preparation of Compound 1399

Compound 1399 was prepared on a 50 μmol scale. The yield of the product was 7.2 mg, and its estimated purity by LCMS analysis was 100%. Analysis condition A: Retention time=2.08 min; ESI-MS(+) m/z [M+2H]²⁺: 942.2.

### Preparation of Compound 1400

Compound 1400 was prepared on a 50 μmol scale. The yield of the product was 15.5 mg, and its estimated purity by LCMS analysis was 99.1%. Analysis condition B: Retention time=1.58 min; ESI-MS(+) m/z [M+H]⁺: 1891.2.

### Preparation of Compound 1401

Compound 1401 was prepared on a 50 μmol scale. The yield of the product was 9.5 mg, and its estimated purity by LCMS analysis was 100%. Analysis condition A: Retention time=1.65 min; ESI-MS(+) m/z [M+H]⁺: 1892.2.

### Preparation of Compound 1402

Compound 1402 was prepared on a 50 μmol scale. The yield of the product was 7.3 mg, and its estimated purity by LCMS analysis was 95.2%. Analysis condition B: Retention time=1.49 min; ESI-MS(+) m/z [M+2H]²⁺: 973.1.

### Preparation of Compound 1403

Compound 1403 was prepared on a 50 μmol scale. The yield of the product was 5.8 mg, and its estimated purity by LCMS analysis was 82.4%. Analysis condition A: Retention time=1.58 min; ESI-MS(+) m/z [M+2H]²⁺: 942.3.

### Preparation of Compound 1404

Compound 1404 was prepared on a 50 μmol scale. The yield of the product was 3.6 mg, and its estimated purity by LCMS analysis was 83.1%. Analysis condition A: Retention time=1.41 min; ESI-MS(+) m/z [M+3H]³⁺: 634.4.

### Preparation of Compound 1405

Compound 1405 was prepared on a 50 μmol scale. The yield of the product was 6.6 mg, and its estimated purity by LCMS analysis was 87.8%. Analysis condition A: Retention time=1.55 min; ESI-MS(+) m/z [M+2H]²⁺: 908.1.

### Preparation of Compound 1406

Compound 1406 was prepared on a 50 μmol scale. The yield of the product was 6.8 mg, and its estimated purity by LCMS analysis was 96%. Analysis condition B: Retention time=1.59 min; ESI-MS(+) m/z [M+2H]²⁺: 953.1.

### Preparation of Compound 1407

Compound 1407 was prepared on a 50 μmol scale. The yield of the product was 3.8 mg, and its estimated purity by LCMS analysis was 93.1%. Analysis condition B: Retention time=1.58 min; ESI-MS(+) m/z [M+2H]²⁺: 951.1.

### Preparation of Compound 1408

Compound 1408 was prepared on a 50 μmol scale. The yield of the product was 3.2 mg, and its estimated purity by LCMS analysis was 84.2%. Analysis condition A: Retention time=1.58 min; ESI-MS(+) m/z [M+2H]²⁺: 942.2.

### Preparation of Compound 1409

Compound 1409 was prepared on a 50 μmol scale. The yield of the product was 2.9 mg, and its estimated purity by LCMS analysis was 83.4%. Analysis condition B: Retention time=1.51, 1.55 min; ESI-MS(+) m/z [M+2H]²⁺: 938.22, 937.5.

### Preparation of Compound 1410

Compound 1410 was prepared on a 50 μmol scale. The yield of the product was 5.6 mg, and its estimated purity by LCMS analysis was 90%. Analysis condition A: Retention time=1.56 min; ESI-MS(+) m/z [M+2H]²⁺: 939.2.

### Preparation of Compound 1411

Compound 1411 was prepared on a 50 μmol scale. The yield of the product was 3.5 mg, and its estimated purity by LCMS analysis was 94.9%. Analysis condition A: Retention time=1.52 min; ESI-MS(+) m/z [M+H]⁺: 1919.2.

### Preparation of Compound 1412

Compound 1412 was prepared on a 50 μmol scale. The yield of the product was 12.1 mg, and its estimated purity by LCMS analysis was 88.8%. Analysis condition B: Retention time=1.45 min; ESI-MS(+) m/z [M+2H]²⁺: 929.4.

### Preparation of Compound 1413

Compound 1413 was prepared on a 50 μmol scale. The yield of the product was 2.2 mg, and its estimated purity by LCMS analysis was 95.5%. Analysis condition A: Retention time=1.38 min; ESI-MS(+) m/z [M+2H]²⁺: 975.2.

### Preparation of Compound 1414

Compound 1414 was prepared on a 50 μmol scale. The yield of the product was 1.7 mg, and its estimated purity by LCMS analysis was 92.8%. Analysis condition A: Retention time=1.47 min; ESI-MS(+) m/z [M+2H]²⁺: 915.4.

### Preparation of Compound 1415

Compound 1415 was prepared on a 50 μmol scale. The yield of the product was 16.5 mg, and its estimated purity by LCMS analysis was 84.7%. Analysis condition B: Retention time=1.47, 1.5 min; ESI-MS(+) m/z [M+H]⁺: 1818.

### Preparation of Compound 1416

Compound 1416 was prepared on a 40 μmol scale. The yield of the product was 3.9 mg, and its estimated purity by LCMS analysis was 96%. Analysis condition B: Retention time=1.44 min; ESI-MS(+) m/z [M+2H]²⁺: 922.2.

### Preparation of Compound 1417

Compound 1417 was prepared on a 50 μmol scale. The yield of the product was 19.2 mg, and its estimated purity by LCMS analysis was 97.3%. Analysis condition B: Retention time=1.46 min; ESI-MS(+) m/z [M+2H]²⁺: 927.3.

### Preparation of Compound 1418

Compound 1418 was prepared on a 50 μmol scale. The yield of the product was 16.7 mg, and its estimated purity by LCMS analysis was 95.5%. Analysis condition B: Retention time=1.64 min; ESI-MS(+) m/z [M+H]⁺: 1849.

### Preparation of Compound 1419

Compound 1419 was prepared on a 50 μmol scale. The yield of the product was 15.1 mg, and its estimated purity by LCMS analysis was 96.2%. Analysis condition A: Retention time=1.74 min; ESI-MS(+) m/z [M+H]⁺: 1911.

### Preparation of Compound 1420

Compound 1420 was prepared on a 50 μmol scale. The yield of the product was 12.5 mg, and its estimated purity by LCMS analysis was 91.2%. Analysis condition B: Retention time=1.57 min; ESI-MS(+) m/z [M+2H]²⁺: 941.

### Preparation of Compound 1421

Compound 1421 was prepared on a 50 μmol scale. The yield of the product was 13.4 mg, and its estimated purity by LCMS analysis was 94.4%. Analysis condition B: Retention time=1.57 min; ESI-MS(+) m/z [M+H]⁺: 1967.9.

### Preparation of Compound 1422

Compound 1422 was prepared on a 50 μmol scale. The yield of the product was 10.6 mg, and its estimated purity by LCMS analysis was 81.4%. Analysis condition A: Retention time=1.81, 1.89 min; ESI-MS(+) m/z [M+H]⁺: 1848.

### Preparation of Compound 1423

Compound 1423 was prepared on a 40 μmol scale. The yield of the product was 17.9 mg, and its estimated purity by LCMS analysis was 98.7%. Analysis condition B: Retention time=1.52 min; ESI-MS(+) m/z [M+H]⁺: 1866.9.

### Preparation of Compound 1424

Compound 1424 was prepared on a 40 μmol scale. The yield of the product was 17.2 mg, and its estimated purity by LCMS analysis was 90.8%. Analysis condition A: Retention time=1.51 min: ESI-MS(+) m/z [M+2H]²⁺: 927.1.

### Preparation of Compound 1425

Compound 1425 was prepared on a 40 μmol scale. The yield of the product was 21.8 mg, and its estimated purity by LCMS analysis was 99.1%. Analysis condition B: Retention time=1.51 min; ESI-MS(+) m/z [M+H]⁺: 1854.9.

### Preparation of Compound 1426

Compound 1426 was prepared, using Rink Resin on a 50 μmol scale, following the general synthetic sequence described for the preparation of Compound 1948. The crude material was purified via preparative LC/MS with the following conditions: Column: XBridge C18, 200 mm×30 mm, 5-μm particles; Mobile Phase A: 5:95 acetonitrile:water with 10-mM ammonium acetate; Mobile Phase B: 95:5 acetonitrile:water with 10-mM ammonium acetate; Gradient: a 0-minute hold at 20% B, 20-60% B over 20 minutes, then a 2-minute hold at 100% B; Flow Rate: 45 mL/min; Column Temperature: 25 C. Fraction collection was triggered by MS and UV signals. Fractions containing the desired product were combined and dried via centrifugal evaporation. The material was further purified via preparative LC/MS with the following conditions: Column: XBridge C18, 150 mm×30 mm, 5-μm particles; Mobile Phase A: 5:95 acetonitrile:water with 0.05% trifluoroacetic acid; Mobile Phase B: 95:5 acetonitrile:water with 0.05% trifluoroacetic acid; Gradient: a 0-minute hold at 28% B, 28-68% B over 20 minutes, then a 2-minute hold at 100% B; Flow Rate: 40 mL/min; Column Temperature: 25 C. Fraction collection was triggered by MS and UV signals. Fractions containing the desired product were combined and dried via centrifugal evaporation. The yield of the product was 9.7 mg, and its estimated purity by LCMS analysis was 98.8%.

Analysis condition A: Retention time=1.65 min; ESI-MS(+) m/z [M+H]⁺: 1947.1.

### Preparation of Compound 1427

Compound 1427 was prepared on a 40 μmol scale. The yield of the product was 19.7 mg, and its estimated purity by LCMS analysis was 85.5%. Analysis condition B: Retention time=1.6 min; ESI-MS(+) m/z [M+3H]³⁺: 610.3.

### Preparation of Compound 1428

Compound 1428 was prepared on a 50 μmol scale. The yield of the product was 26.7 mg, and its estimated purity by LCMS analysis was 98.7%. Analysis condition B: Retention time=1.6, 1.66 min; ESI-MS(+) m/z [M+H]⁺: 1791.22, 1790.24.

### Preparation of Compound 1429

Compound 1429 was prepared on a 50 μmol scale. The yield of the product was 13 mg, and its estimated purity by LCMS analysis was 95.3%. Analysis condition A: Retention time=1.4 min; ESI-MS(+) m/z [M+2H]²⁺: 939.2.

### Preparation of Compound 1430

Compound 1430 was prepared on a 50 μmol scale. The yield of the product was 8.3 mg, and its estimated purity by LCMS analysis was 0%. Analysis condition B: Retention time=1.42 min; ESI-MS(+) m/z [M+2H]²⁺: 951.1.

### Preparation of Compound 1431

Compound 1431 was prepared on a 50 μmol scale. The yield of the product was 6.3 mg, and its estimated purity by LCMS analysis was 97%. Analysis condition B: Retention time=1.57 min; ESI-MS(+) m/z [M+H]⁺: 1937.9.

### Preparation of Compound 1432

Compound 1432 was prepared on a 40 μmol scale. The yield of the product was 17.3 mg, and its estimated purity by LCMS analysis was 97.8%. Analysis condition B: Retention time=1.5 min; ESI-MS(+) m/z [M+H]⁺: 1862.9.

### Preparation of Compound 1433

Compound 1433 was prepared on a 40 μmol scale. The yield of the product was 15 mg, and its estimated purity by LCMS analysis was 92.6%. Analysis condition A: Retention time=1.61 min; ESI-MS(+) m/z [M+2H]²⁺: 931.9.

### Preparation of Compound 1434

Compound 1434 was prepared on a 40 μmol scale. The yield of the product was 11.2 mg, and its estimated purity by LCMS analysis was 96.8%. Analysis condition B: Retention time=1.6 min; ESI-MS(+) m/z [M+H]⁺: 1904.8.

### Preparation of Compound 1435

Compound 1435 was prepared on a 40 μmol scale. The yield of the product was 16.3 mg, and its estimated purity by LCMS analysis was 91%. Analysis condition B: Retention time=1.63 min; ESI-MS(+) m/z [M+H]⁺: 1825.2.

### Preparation of Compound 1436

Compound 1436 was prepared on a 40 μmol scale. The yield of the product was 13.4 mg, and its estimated purity by LCMS analysis was 95.6%. Analysis condition A: Retention time=1.66 min; ESI-MS(+) m/z [M+H]⁺: 1824.6.

### Preparation of Compound 1437

Compound 1437 was prepared on a 50 μmol scale. The yield of the product was 8.2 mg, and its estimated purity by LCMS analysis was 92.6%. Analysis condition A: Retention time=1.5 min; ESI-MS(+) m/z [M+H]⁺: 1906.2.

### Preparation of Compound 1438

Compound 1438 was prepared on a 40 μmol scale. The yield of the product was 5.6 mg, and its estimated purity by LCMS analysis was 84%. Analysis condition B: Retention time=1.42 min; ESI-MS(+) m/z [M+H]⁺: 1912.3.

### Preparation of Compound 1439

Compound 1439 was prepared on a 40 μmol scale. The yield of the product was 32.5 mg, and its estimated purity by LCMS analysis was 81%. Analysis condition B: Retention time=1.37, 1.44 min; ESI-MS(+) m/z [M+H]⁺: 1881.1.

### Preparation of Compound 1440

Compound 1440 was prepared on a 40 μmol scale. The yield of the product was 29.2 mg, and its estimated purity by LCMS analysis was 85.7%. Analysis condition B: Retention time=1.52 min; ESI-MS(+) m/z [M+2H]²⁺: 936.2.

### Preparation of Compound 1441

Compound 1441 was prepared on a 40 μmol scale. The yield of the product was 17.7 mg, and its estimated purity by LCMS analysis was 96.9%. Analysis condition B: Retention time=1.66, 1.74 min; ESI-MS(+) m/z [M+H]⁺: 1833.6.

### Preparation of Compound 1442

Compound 1442 was prepared on a 40 μmol scale. The yield of the product was 14.7 mg, and its estimated purity by LCMS analysis was 93.4%. Analysis condition A: Retention time=1.67 min; ESI-MS(+) m/z [M+H]⁺: 1818.2.

### Preparation of Compound 1443

Compound 1443 was prepared on a 40 μmol scale. The yield of the product was 26.6 mg, and its estimated purity by LCMS analysis was 97.3%. Analysis condition A: Retention time=1.45 min; ESI-MS(+) m/z [M+2H]²⁺: 997.2.

### Preparation of Compound 1444

Compound 1444 was prepared on a 40 μmol scale. The yield of the product was 25.6 mg, and its estimated purity by LCMS analysis was 95.9%. Analysis condition A: Retention time=1.72 min; ESI-MS(+) m/z [M+2H]²⁺: 941.1.

### Preparation of Compound 1445

Compound 1445 was prepared on a 40 μmol scale. The yield of the product was 26.9 mg, and its estimated purity by LCMS analysis was 96.8%. Analysis condition B: Retention time=1.4, 1.43 min; ESI-MS(+) m/z [M+H]⁺: 1890.6.

### Preparation of Compound 1446

Compound 1446 was prepared on a 40 μmol scale. The yield of the product was 0.5 mg, and its estimated purity by LCMS analysis was 93.4%. Analysis condition A: Retention time=1.51 min; ESI-MS(+) m/z [M+2H]²⁺: 972.5.

### Preparation of Compound 1447

Compound 1447 was prepared on a 40 μmol scale. The yield of the product was 8.9 mg, and its estimated purity by LCMS analysis was 82%. Analysis condition A: Retention time=1.39 min; ESI-MS(+) m/z [M+2H]²⁺: 937.1.

### Preparation of Compound 1448

Compound 1448 was prepared on a 40 μmol scale. The yield of the product was 8.7 mg, and its estimated purity by LCMS analysis was 96.7%. Analysis condition B: Retention time=1.46 min; ESI-MS(+) m/z [M+H]⁺: 1920.9.

Compound 1449 was prepared on a 40 μmol scale. The yield of the product was 4.7 mg, and its estimated purity by LCMS analysis was 94%. Analysis condition B: Retention time=1.32 min; ESI-MS(+) m/z [M+2H]²⁺: 959.1.

### Preparation of Compound 1450

Compound 1450 was prepared on a 40 μmol scale. The yield of the product was 31.2 mg, and its estimated purity by LCMS analysis was 96.7%. Analysis condition B: Retention time=1.3 min; ESI-MS(+) m/z [M+3H]³⁺: 644.4.

### Preparation of Compound 1451

Compound 1451 was prepared on a 40 μmol scale. The yield of the product was 19.6 mg, and its estimated purity by LCMS analysis was 92.2%. Analysis condition B: Retention time=1.3 min; ESI-MS(+) m/z [M+2H]²⁺: 951.

### Preparation of Compound 1452

Compound 1452 was prepared on a 40 μmol scale. The yield of the product was 13.5 mg, and its estimated purity by LCMS analysis was 100%. Analysis condition A: Retention time=1.42 min; ESI-MS(+) m/z [M+2H]²⁺: 965.

### Preparation of Compound 1453

Compound 1453 was prepared on a 40 μmol scale. The yield of the product was 11.5 mg, and its estimated purity by LCMS analysis was 93.3%. Analysis condition B: Retention time=1.37 min; ESI-MS(+) m/z [M+2H]²⁺: 944.

### Preparation of Compound 1454

Compound 1454 was prepared on a 40 μmol scale. The yield of the product was 17.4 mg, and its estimated purity by LCMS analysis was 96.1%. Analysis condition B: Retention time=1.47 min; ESI-MS(+) m/z [M+2H]²⁺: 937.9.

### Preparation of Compound 1455

Compound 1455 was prepared on a 40 μmol scale. The yield of the product was 19.1 mg, and its estimated purity by LCMS analysis was 100%. Analysis condition A: Retention time=1.63 min; ESI-MS(+) m/z [M+H]⁺: 1882.1.

### Preparation of Compound 1456

Compound 1456 was prepared on a 40 μmol scale. The yield of the product was 1.5 mg, and its estimated purity by LCMS analysis was 93%. Analysis condition B: Retention time=1.46 min; ESI-MS(+) m/z [M+H]⁺: 1905.1.

### Preparation of Compound 1457

Compound 1457 was prepared on a 40 μmol scale. The yield of the product was 6.6 mg, and its estimated purity by LCMS analysis was 97.1%. Analysis condition B: Retention time=1.45, 1.52 min; ESI-MS(+) m/z [M+H]⁺: 1875.3.

### Preparation of Compound 1458

Compound 1458 was prepared on a 40 μmol scale. The yield of the product was 5.8 mg, and its estimated purity by LCMS analysis was 90.6%. Analysis condition A: Retention time=1.51 min; ESI-MS(+) m/z [M+3H]³⁺: 643.

### Preparation of Compound 1459

Compound 1459 was prepared on a 40 μmol scale. The yield of the product was 6.2 mg, and its estimated purity by LCMS analysis was 100%. Analysis condition A: Retention time=1.36 min; ESI-MS(+) m/z [M+3H]³⁺: 632.8.

### Preparation of Compound 1460

Compound 1460 was prepared on a 40 μmol scale. The yield of the product was 10.6 mg, and its estimated purity by LCMS analysis was 94.2%. Analysis condition B: Retention time=1.29 min; ESI-MS(+) m/z [M+3H]³⁺: 631.4.

### Preparation of Compound 1461

Compound 1461 was prepared on a 40 μmol scale. The yield of the product was 14.2 mg, and its estimated purity by LCMS analysis was 93.5%. Analysis condition B: Retention time=1.32 min; ESI-MS(+) m/z [M+2H]²⁺: 943.4.

### Preparation of Compound 1462

Compound 1462 was prepared on a 40 μmol scale. The yield of the product was 20.7 mg, and its estimated purity by LCMS analysis was 95.2%. Analysis condition B: Retention time=1.54 min; ESI-MS(+) m/z [M+2H]²⁺: 925.5.

### Preparation of Compound 1463

Compound 1463 was prepared on a 40 μmol scale. The yield of the product was 6.9 mg, and its estimated purity by LCMS analysis was 92.8%. Analysis condition A: Retention time=1.54 min; ESI-MS(+) m/z [M+2H]²⁺: 920.4.

### Preparation of Compound 1464

Compound 1464 was prepared on a 40 μmol scale. The yield of the product was 6.5 mg, and its estimated purity by LCMS analysis was 98.8%. Analysis condition B: Retention time=1.57 min; ESI-MS(+) m/z [M+2H]²⁺: 889.3.

### Preparation of Compound 1465

Compound 1465 was prepared on a 40 μmol scale. The yield of the product was 12.1 mg, and its estimated purity by LCMS analysis was 95.4%. Analysis condition A: Retention time=1.52 min; ESI-MS(+) m/z [M+2H]²⁺: 894.4.

### Preparation of Compound 1466

Compound 1466 was prepared on a 50 μmol scale. The yield of the product was 10 mg, and its estimated purity by LCMS analysis was 100%. Analysis condition A: Retention time=1.73 min; ESI-MS(+) m/z [M+2H]²⁺: 946.

### Preparation of Compound 1467

Compound 1467 was prepared on a 50 μmol scale. The yield of the product was 11.1 mg, and its estimated purity by LCMS analysis was 100%. Analysis condition A: Retention time=1.73 min; ESI-MS(+) m/z [M+2H]²⁺: 953.

### Preparation of Compound 1468

Compound 1468 was prepared on a 50 μmol scale. The yield of the product was 12.6 mg, and its estimated purity by LCMS analysis was 100%. Analysis condition A: Retention time=1.71 min; ESI-MS(+) m/z [M+2H]²⁺: 939.1.

### Preparation of Compound 1469

Compound 1469 was prepared on a 50 μmol scale. The yield of the product was 14 mg, and its estimated purity by LCMS analysis was 100%. Analysis condition A: Retention time=1.74 min; ESI-MS(+) m/z [M+2H]²⁺: 939.1.

### Preparation of Compound 1470

Compound 1470 was prepared on a 50 μmol scale. The yield of the product was 12.7 mg, and its estimated purity by LCMS analysis was 99.3%. Analysis condition B: Retention time=1.73 min; ESI-MS(+) m/z [M+2H]²⁺: 939.1.

### Preparation of Compound 1471

Compound 1471 was prepared on a 50 μmol scale. The yield of the product was 16.1 mg, and its estimated purity by LCMS analysis was 98.2%. Analysis condition B: Retention time=1.76 min; ESI-MS(+) m/z [M+2H]²⁺: 946.1.

### Preparation of Compound 1472

Compound 1472 was prepared on a 50 μmol scale. The yield of the product was 18.2 mg, and its estimated purity by LCMS analysis was 99.2%. Analysis condition A: Retention time=1.71 min; ESI-MS(+) m/z [M+2H]²⁺: 931.2.

### Preparation of Compound 1473

Compound 1473 was prepared on a 50 μmol scale. The yield of the product was 2 mg, and its estimated purity by LCMS analysis was 100%. Analysis condition A: Retention time=1.55, 1.61 min; ESI-MS(+) m/z [M+H]⁺: 1916.

### Preparation of Compound 1474

Compound 1474 was prepared on a 50 μmol scale. The yield of the product was 12.1 mg, and its estimated purity by LCMS analysis was 98.4%. Analysis condition A: Retention time=1.37 min; ESI-MS(+) m/z [M+2H]²⁺: 951.1.

### Preparation of Compound 1475

Compound 1475 was prepared on a 50 μmol scale. The yield of the product was 14.3 mg, and its estimated purity by LCMS analysis was 100%. Analysis condition A: Retention time=1.43 min; ESI-MS(+) m/z [M+2H]²⁺: 958.

### Preparation of Compound 1476

Compound 1476 was prepared on a 50 μmol scale. The yield of the product was 24.8 mg, and its estimated purity by LCMS analysis was 98.7%. Analysis condition B: Retention time=1.68 min; ESI-MS(+) m/z [M+2H]²⁺: 925.1.

### Preparation of Compound 1477

Compound 1477 was prepared on a 50 μmol scale. The yield of the product was 10.6 mg, and its estimated purity by LCMS analysis was 94.6%. Analysis condition A: Retention time=1.49 min; ESI-MS(+) m/z [M+2H]²⁺: 918.

### Preparation of Compound 1478

Compound 1478 was prepared on a 50 μmol scale. The yield of the product was 17 mg, and its estimated purity by LCMS analysis was 94.8%. Analysis condition B: Retention time=1.59 min; ESI-MS(+) m/z [M+2H]²⁺: 925.2.

### Preparation of Compound 1479

Compound 1479 was prepared on a 50 μmol scale. The yield of the product was 14.6 mg, and its estimated purity by LCMS analysis was 98.5%. Analysis condition A: Retention time=1.51 min; ESI-MS(+) m/z [M+2H]²⁺: 911.2.

### Preparation of Compound 1480

Compound 1480 was prepared on a 50 μmol scale. The yield of the product was 8.3 mg, and its estimated purity by LCMS analysis was 93.2%. Analysis condition A: Retention time=1.51 min; ESI-MS(+) m/z [M+2H]²⁺: 935.9.

### Preparation of Compound 1481

Compound 1481 was prepared on a 50 μmol scale. The yield of the product was 7 mg, and its estimated purity by LCMS analysis was 95.9%. Analysis condition A: Retention time=1.47 min; ESI-MS(+) m/z [M+2H]²⁺: 955.4.

### Preparation of Compound 1482

Compound 1482 was prepared on a 50 μmol scale. The yield of the product was 9.2 mg, and its estimated purity by LCMS analysis was 100%. Analysis condition A: Retention time=1.46 min; ESI-MS(+) m/z [M+2H]²⁺: 948.3.

### Preparation of Compound 1483

Compound 1483 was prepared on a 50 μmol scale. The yield of the product was 18.9 mg, and its estimated purity by LCMS analysis was 98.9%. Analysis condition B: Retention time=1.36 min; ESI-MS(+) m/z [M+2H]²⁺: 942.1.

### Preparation of Compound 1484

Compound 1484 was prepared on a 50 μmol scale. The yield of the product was 15.4 mg, and its estimated purity by LCMS analysis was 98.1%. Analysis condition B: Retention time=1.41 min; ESI-MS(+) m/z [M+2H]²⁺: 948.1.

### Preparation of Compound 1485

Compound 1485 was prepared on a 50 μmol scale. The yield of the product was 7.8 mg, and its estimated purity by LCMS analysis was 100%. Analysis condition A: Retention time=1.39 min; ESI-MS(+) m/z [M+3H]³⁺: 638.

### Preparation of Compound 1486

Compound 1486 was prepared on a 50 μmol scale. The yield of the product was 23 mg, and its estimated purity by LCMS analysis was 85.7%. Analysis condition B: Retention time=1.41 min; ESI-MS(+) m/z [M+2H]²⁺: 1024.1.

### Preparation of Compound 1487

Compound 1487 was prepared on a 50 μmol scale. The yield of the product was 20.2 mg, and its estimated purity by LCMS analysis was 97.5%. Analysis condition B: Retention time=1.5 min; ESI-MS(+) m/z [M+2H]²⁺: 987.1.

### Preparation of Compound 1488

Compound 1488 was prepared on a 50 μmol scale. The yield of the product was 10.9 mg, and its estimated purity by LCMS analysis was 89.5%. Analysis condition A: Retention time=1.64 min; ESI-MS(+) m/z [M+2H]²⁺: 943.2.

### Preparation of Compound 1489

Compound 1489 was prepared on a 50 μmol scale. The yield of the product was 9.9 mg, and its estimated purity by LCMS analysis was 100%. Analysis condition A: Retention time=1.57 min; ESI-MS(+) m/z [M+2H]²⁺: 1011.1.

### Preparation of Compound 1490

Compound 1490 was prepared on a 50 μmol scale. The yield of the product was 8.3 mg, and its estimated purity by LCMS analysis was 98.7%. Analysis condition A: Retention time=1.51 min; ESI-MS(+) m/z [M+2H]²⁺: 957.

### Preparation of Compound 1491

Compound 1491 was prepared on a 50 μmol scale. The yield of the product was 11.2 mg, and its estimated purity by LCMS analysis was 98.3%. Analysis condition A: Retention time=1.72 min; ESI-MS(+) m/z [M+2H]²⁺: 973.3.

### Preparation of Compound 1492

Compound 1492 was prepared on a 50 μmol scale. The yield of the product was 5.9 mg, and its estimated purity by LCMS analysis was 83.3%. Analysis condition A: Retention time=1.8 min; ESI-MS(+) m/z [M+2H]²⁺: 963.1.

### Preparation of Compound 1493

Compound 1493 was prepared on a 50 μmol scale. The yield of the product was 7.7 mg, and its estimated purity by LCMS analysis was 87.4%. Analysis condition A: Retention time=1.68 min; ESI-MS(+) m/z [M+2H]²⁺: 1031.

### Preparation of Compound 1494

Compound 1494 was prepared on a 50 μmol scale. The yield of the product was 6.4 mg, and its estimated purity by LCMS analysis was 100%. Analysis condition A: Retention time=1.9 min; ESI-MS(+) m/z [M+2H]²⁺: 994.1.

### Preparation of Compound 1495

Compound 1495 was prepared on a 50 μmol scale. The yield of the product was 11.6 mg, and its estimated purity by LCMS analysis was 98.6%. Analysis condition B: Retention time=1.7 min; ESI-MS(+) m/z [M+2H]²⁺: 977.

### Preparation of Compound 1496

Compound 1496 was prepared on a 50 μmol scale. The yield of the product was 9.3 mg, and its estimated purity by LCMS analysis was 91.1%. Analysis condition B: Retention time=1.65 min; ESI-MS(+) m/z [M+H]⁺: 1886.3.

### Preparation of Compound 1497

Compound 1497 was prepared on a 50 μmol scale. The yield of the product was 14.4 mg, and its estimated purity by LCMS analysis was 100%. Analysis condition A: Retention time=1.62 min; ESI-MS(+) m/z [M+2H]²⁺: 1011.2.

### Preparation of Compound 1498

Compound 1498 was prepared on a 50 μmol scale. The yield of the product was 7.3 mg, and its estimated purity by LCMS analysis was 100%. Analysis condition B: Retention time=1.62 min; ESI-MS(+) m/z [M+3H]³⁺: 638.5.

### Preparation of Compound 1499

Compound 1499 was prepared on a 50 μmol scale. The yield of the product was 10.6 mg, and its estimated purity by LCMS analysis was 98.2%. Analysis condition B: Retention time=1.84 min; ESI-MS(+) m/z [M+H]⁺: 1947.3.

### Preparation of Compound 1500

Compound 1500 was prepared on a 50 μmol scale. The yield of the product was 15.4 mg, and its estimated purity by LCMS analysis was 95.4%. Analysis condition B: Retention time=1.52 min; ESI-MS(+) m/z [M+2H]²¹: 936.2.

### Preparation of Compound 1501

Compound 1501 was prepared on a 50 μmol scale. The yield of the product was 20.2 mg, and its estimated purity by LCMS analysis was 97.8%. Analysis condition A: Retention time=1.55 min; ESI-MS(+) m/z [M+2H]²⁺: 1004.1.

### Preparation of Compound 1502

Compound 1502 was prepared on a 50 μmol scale. The yield of the product was 42.7 mg, and its estimated purity by LCMS analysis was 98.7%. Analysis condition A: Retention time=1.68 min; ESI-MS(+) m/z [M+2H]²⁺: 967.

### Preparation of Compound 1503

Compound 1503 was prepared on a 50 μmol scale. The yield of the product was 16.7 mg, and its estimated purity by LCMS analysis was 96.7%. Analysis condition A: Retention time=1.63 min; ESI-MS(+) m/z [M+2H]²⁺: 985.1.

### Preparation of Compound 1504

Compound 1504 was prepared on a 50 μmol scale. The yield of the product was 19.7 mg, and its estimated purity by LCMS analysis was 100%. Analysis condition A: Retention time=1.62 min; ESI-MS(+) m/z [M+2H]²⁺: 1052.9.

### Preparation of Compound 1505

Compound 1505 was prepared on a 50 μmol scale. The yield of the product was 11.5 mg, and its estimated purity by LCMS analysis was 97%. Analysis condition B: Retention time=1.77 min; ESI-MS(+) m/z [M+2H]²⁺: 1016.3.

### Preparation of Compound 1506

Compound 1506 was prepared on a 50 μmol scale. The yield of the product was 18.3 mg, and its estimated purity by LCMS analysis was 90.3%. Analysis condition B: Retention time=1.58 min; ESI-MS(+) m/z [M+3H]³⁺: 619.1.

### Preparation of Compound 1507

Compound 1507 was prepared on a 50 μmol scale. The yield of the product was 21.1 mg, and its estimated purity by LCMS analysis was 99.2%. Analysis condition A: Retention time=1.61 min; ESI-MS(+) m/z [M+2H]²⁺: 996.1.

### Preparation of Compound 1508

Compound 1508 was prepared on a 50 μmol scale. The yield of the product was 13.2 mg, and its estimated purity by LCMS analysis was 98.6%. Analysis condition A: Retention time=1.84 min; ESI-MS(+) m/z [M+H]⁺: 1890.5.

### Preparation of Compound 1509

Compound 1509 was prepared on a 50 μmol scale. The yield of the product was 22.6 mg, and its estimated purity by LCMS analysis was 99.3%. Analysis condition B: Retention time=1.83 min; ESI-MS(+) m/z [M+H]⁺: 1934.1.

### Preparation of Compound 1510

Compound 1510 was prepared on a 50 μmol scale. The yield of the product was 23 mg, and its estimated purity by LCMS analysis was 100%. Analysis condition A: Retention time=1.7 min; ESI-MS(+) m/z [M+H]⁺: 1906.5.

### Preparation of Compound 1511

Compound 1511 was prepared on a 50 μmol scale. The yield of the product was 13.1 mg, and its estimated purity by LCMS analysis was 98%. Analysis condition B: Retention time=1.8 min; ESI-MS(+) m/z [M+H]⁺: 1876.1.

### Preparation of Compound 1512

Compound 1512 was prepared on a 50 μmol scale. The yield of the product was 16.7 mg, and its estimated purity by LCMS analysis was 97%. Analysis condition A: Retention time=1.67 min; ESI-MS(+) m/z [M+H]⁺: 1920.1.

### Preparation of Compound 1513

Compound 1513 was prepared on a 50 μmol scale. The yield of the product was 21.3 mg, and its estimated purity by LCMS analysis was 100%. Analysis condition A: Retention time=1.63 min; ESI-MS(+) m/z [M+H]⁺: 1892.

### Preparation of Compound 1514

Compound 1514 was prepared on a 50 μmol scale. The yield of the product was 21 mg, and its estimated purity by LCMS analysis was 98.2%. Analysis condition B: Retention time=1.52 min; ESI-MS(+) m/z [M+2H]²⁺: 963.2.

### Preparation of Compound 1515

Compound 1515 was prepared on a 50 μmol scale. The yield of the product was 16.9 mg, and its estimated purity by LCMS analysis was 93.9%. Analysis condition A: Retention time=1.42 min; ESI-MS(+) m/z [M+H]⁺: 1968.

### Preparation of Compound 1516

Compound 1516 was prepared on a 50 μmol scale. The yield of the product was 29.6 mg, and its estimated purity by LCMS analysis was 100%. Analysis condition A: Retention time=1.8 min; ESI-MS(+) m/z [M+H]⁺: 1867.6.

### Preparation of Compound 1517

Compound 1517 was prepared on a 50 μmol scale. The yield of the product was 22.1 mg, and its estimated purity by LCMS analysis was 98.2%. Analysis condition A: Retention time=1.78 min; ESI-MS(+) m/z [M+H]⁺: 1852.1.

### Preparation of Compound 1518

Compound 1518 was prepared on a 50 μmol scale. The yield of the product was 36.1 mg, and its estimated purity by LCMS analysis was 99.1%. Analysis condition B: Retention time=2.10 min; ESI-MS(+) m/z [M+2H]²⁺: 937.5.

### Preparation of Compound 1519

Compound 1519 was prepared on a 50 μmol scale. The yield of the product was 23.5 mg, and its estimated purity by LCMS analysis was 100%. Analysis condition A: Retention time=1.79 min; ESI-MS(+) m/z [M+2H]²⁺: 938.1.

### Preparation of Compound 1520

Compound 1520 was prepared on a 50 μmol scale. The yield of the product was 18.5 mg, and its estimated purity by LCMS analysis was 98.7%. Analysis condition B: Retention time=1.95 min; ESI-MS(+) m/z [M+2H]²⁺: 938.2.

### Preparation of Compound 1521

Compound 1521 was prepared on a 50 μmol scale. The yield of the product was 15.7 mg, and its estimated purity by LCMS analysis was 100%. Analysis condition A: Retention time=1.94 min; ESI-MS(+) m/z [M+H]⁺: 1891.

### Preparation of Compound 1522

Compound 1522 was prepared on a 50 μmol scale. The yield of the product was 6.3 mg, and its estimated purity by LCMS analysis was 100%. Analysis condition A: Retention time=1.82 min; ESI-MS(+) m/z [M+H]⁺: 1828.9.

### Preparation of Compound 1523

Compound 1523 was prepared on a 50 μmol scale. The yield of the product was 9.3 mg, and its estimated purity by LCMS analysis was 98.8%. Analysis condition B: Retention time=1.9 min; ESI-MS(+) m/z [M+H]⁺: 1892.1.

### Preparation of Compound 1524

Compound 1524 was prepared on a 50 μmol scale. The yield of the product was 21.3 mg, and its estimated purity by LCMS analysis was 98.1%. Analysis condition A: Retention time=1.76 min; ESI-MS(+) m/z [M+H]⁺: 1892.1.

### Preparation of Compound 1525

Compound 1525 was prepared on a 50 μmol scale. The yield of the product was 15.8 mg, and its estimated purity by LCMS analysis was 95.3%. Analysis condition A: Retention time=1.83 min; ESI-MS(+) m/z [M+H]⁺: 1884.9.

### Preparation of Compound 1526

Compound 1526 was prepared on a 50 μmol scale. The yield of the product was 7.6 mg, and its estimated purity by LCMS analysis was 92.2%. Analysis condition A: Retention time=1.59 min; ESI-MS(+) m/z [M+H]⁺: 1934.1.

### Preparation of Compound 1527

Compound 1527 was prepared on a 50 μmol scale. The yield of the product was 10.8 mg, and its estimated purity by LCMS analysis was 98.1%. Analysis condition B: Retention time=1.85 min; ESI-MS(+) m/z [M+2H]²⁺: 927.

### Preparation of Compound 1528

Compound 1528 was prepared on a 50 μmol scale. The yield of the product was 12.8 mg, and its estimated purity by LCMS analysis was 100%. Analysis condition A: Retention time=1.66 min; ESI-MS(+) m/z [M+2H]²⁺: 953.2.

### Preparation of Compound 1529

Compound 1529 was prepared on a 50 μmol scale. The yield of the product was 2.9 mg, and its estimated purity by LCMS analysis was 97.6%. Analysis condition B: Retention time=1.62 min; ESI-MS(+) m/z [M+2H]²⁺: 939.4.

### Preparation of Compound 1530

Compound 1530 was prepared on a 50 μmol scale. The yield of the product was 8.4 mg, and its estimated purity by LCMS analysis was 93.2%. Analysis condition A: Retention time=1.66 min; ESI-MS(+) m/z [M+2H]²⁺: 932.

### Preparation of Compound 1531

Compound 1531 was prepared on a 50 μmol scale. The yield of the product was 12.3 mg, and its estimated purity by LCMS analysis was 90.2%. Analysis condition A: Retention time=1.69 min; ESI-MS(+) m/z [M+2H]²⁺: 925.1.

### Preparation of Compound 1532

Compound 1532 was prepared on a 50 μmol scale. The yield of the product was 30.9 mg, and its estimated purity by LCMS analysis was 87%. Analysis condition B: Retention time=1.68, 1.74 min; ESI-MS(+) m/z [M+H]⁺: 1836.

### Preparation of Compound 1533

Compound 1533 was prepared on a 50 μmol scale. The yield of the product was 2 mg, and its estimated purity by LCMS analysis was 91.7%. Analysis condition B: Retention time=1.72 min; ESI-MS(+) m/z [M+2H]²⁺: 939.3.

### Preparation of Compound 1534

Compound 1534 was prepared on a 50 μmol scale. The yield of the product was 4.7 mg, and its estimated purity by LCMS analysis was 92%. Analysis condition B: Retention time=1.73 min; ESI-MS(+) m/z [M+2H]²⁺: 932.1.

### Preparation of Compound 1535

Compound 1535 was prepared on a 50 μmol scale. The yield of the product was 10.8 mg, and its estimated purity by LCMS analysis was 99%. Analysis condition A: Retention time=1.74 min; ESI-MS(+) m/z [M+2H]²⁺: 925.1.

### Preparation of Compound 1536

Compound 1536 was prepared on a 50 μmol scale. The yield of the product was 3.9 mg, and its estimated purity by LCMS analysis was 92.2%. Analysis condition B: Retention time=1.85 min; ESI-MS(+) m/z [M+2H]²⁺: 960.2.

### Preparation of Compound 1537

Compound 1537 was prepared on a 50 μmol scale. The yield of the product was 8.6 mg, and its estimated purity by LCMS analysis was 99.2%. Analysis condition A: Retention time=1.84 min; ESI-MS(+) m/z [M+2H]²⁺: 953.2.

### Preparation of Compound 1538

Compound 1538 was prepared on a 50 μmol scale. The yield of the product was 6.1 mg, and its estimated purity by LCMS analysis was 94.4%. Analysis condition A: Retention time=1.86 min; ESI-MS(+) m/z [M+2H]²⁺: 946.

### Preparation of Compound 1539

Compound 1539 was prepared on a 50 μmol scale. The yield of the product was 9.6 mg, and its estimated purity by LCMS analysis was 92.6%. Analysis condition B: Retention time=1.92 min; ESI-MS(+) m/z [M+2H]²⁺: 939.1.

### Preparation of Compound 1540

Compound 1540 was prepared on a 50 μmol scale. The yield of the product was 0.8 mg, and its estimated purity by LCMS analysis was 95.7%. Analysis condition A: Retention time=1.66 min; ESI-MS(+) m/z [M+2H]²⁺: 936.3.

### Preparation of Compound 1541

Compound 1541 was prepared on a 40 μmol scale. The yield of the product was 1 mg, and its estimated purity by LCMS analysis was 84.6%. Analysis condition A: Retention time=1.85 min; ESI-MS(+) m/z [M+2H]²⁺: 957.4.

### Preparation of Compound 1542

Compound 1542 was prepared on a 50 μmol scale. The yield of the product was 34.3 mg, and its estimated purity by LCMS analysis was 100%. Analysis condition A: Retention time=1.54 min; ESI-MS(+) m/z [M+H]⁺: 1800.6.

### Preparation of Compound 1543

Compound 1543 was prepared on a 50 μmol scale. The yield of the product was 47.1 mg, and its estimated purity by LCMS analysis was 97.3%. Analysis condition B: Retention time=1.48 min; ESI-MS(+) m/z [M+H]⁺: 1774.7.

### Preparation of Compound 1544

Compound 1544 was prepared on a 50 μmol scale. The yield of the product was 19.3 mg, and its estimated purity by LCMS analysis was 100%. Analysis condition A: Retention time=1.6 min; ESI-MS(+) m/z [M+H]⁺: 1790.2.

### Preparation of Compound 1545

Compound 1545 was prepared on a 50 μmol scale. The yield of the product was 23.4 mg, and its estimated purity by LCMS analysis was 100%. Analysis condition A: Retention time=1.49 min; ESI-MS(+) m/z [M+H]⁺: 1859.2.

### Preparation of Compound 1546

Compound 1546 was prepared on a 50 μmol scale. The yield of the product was 23.9 mg, and its estimated purity by LCMS analysis was 94.6%. Analysis condition B: Retention time=1.36 min; ESI-MS(+) m/z [M+H]⁺: 917.1.

### Preparation of Compound 1547

Compound 1547 was prepared on a 50 μmol scale. The yield of the product was 19.9 mg, and its estimated purity by LCMS analysis was 100%. Analysis condition A: Retention time=1.53 min; ESI-MS(+) m/z [M+H]⁺: 1846.6.

### Preparation of Compound 1548

Compound 1548 was prepared on a 50 μmol scale. The yield of the product was 29.7 mg, and its estimated purity by LCMS analysis was 100%. Analysis condition A: Retention time=1.48 min; ESI-MS(+) m/z [M+H]⁺: 1978.3.

### Preparation of Compound 1549

Compound 1549 was prepared on a 50 μmol scale. The yield of the product was 16.6 mg, and its estimated purity by LCMS analysis was 91.2%. Analysis condition A: Retention time=1.62 min; ESI-MS(+) m/z [M+2H]²⁺: 901.

### Preparation of Compound 1550

Compound 1550 was prepared on a 50 μmol scale. The yield of the product was 36.2 mg, and its estimated purity by LCMS analysis was 88.3%. Analysis condition A: Retention time=1.4 min; ESI-MS(+) m/z [M+2H]²⁺: 1004.1.

### Preparation of Compound 1551

Compound 1551 was prepared on a 50 μmol scale. The yield of the product was 37.3 mg, and its estimated purity by LCMS analysis was 93.5%. Analysis condition A: Retention time=1.54 min; ESI-MS(+) m/z [M+H]⁺: 1935.6.

### Preparation of Compound 1552

Compound 1552 was prepared on a 50 μmol scale. The yield of the product was 17.3 mg, and its estimated purity by LCMS analysis was 86%. Analysis condition B: Retention time=1.72 min; ESI-MS(+) m/z [M+2H]²⁺: 937.1.

### Preparation of Compound 1553

Compound 1553 was prepared on a 50 μmol scale. The yield of the product was 32.8 mg, and its estimated purity by LCMS analysis was 91.3%. Analysis condition A: Retention time=1.52 min; ESI-MS(+) m/z [M+H]⁺: 1952.2.

### Preparation of Compound 1554

Compound 1554 was prepared on a 50 μmol scale. The yield of the product was 23.4 mg, and its estimated purity by LCMS analysis was 100%. Analysis condition A: Retention time=1.5 min; ESI-MS(+) m/z [M+2H]²⁺: 977.2.

### Preparation of Compound 1555

Compound 1555 was prepared on a 50 μmol scale. The yield of the product was 22.2 mg, and its estimated purity by LCMS analysis was 88.2%. Analysis condition A: Retention time=1.76 min; ESI-MS(+) m/z [M+H]⁺: 1774.8.

### Preparation of Compound 1556

Compound 1556 was prepared on a 50 μmol scale. The yield of the product was 39.6 mg, and its estimated purity by LCMS analysis was 94.1%. Analysis condition A: Retention time=1.42 min; ESI-MS(+) m/z [M+H]⁺: 1983.

### Preparation of Compound 1557

Compound 1557 was prepared on a 50 μmol scale. The yield of the product was 12.3 mg, and its estimated purity by LCMS analysis was 97.8%. Analysis condition A: Retention time=1.44 min; ESI-MS(+) m/z [M+2H]²⁺: 956.1.

### Preparation of Compound 1558

Compound 1558 was prepared on a 50 μmol scale. The yield of the product was 15.9 mg, and its estimated purity by LCMS analysis was 81.3%. Analysis condition B: Retention time=1.78 min; ESI-MS(+) m/z [M+2H]²⁺: 925.2.

### Preparation of Compound 1559

Compound 1559 was prepared on a 50 μmol scale. The yield of the product was 52.2 mg, and its estimated purity by LCMS analysis was 92.1%. Analysis condition A: Retention time=1.53 min; ESI-MS(+) m/z [M+H]⁺: 1910.2.

### Preparation of Compound 1560

Compound 1560 was prepared on a 50 μmol scale. The yield of the product was 29 mg, and its estimated purity by LCMS analysis was 100%. Analysis condition B: Retention time=1.59 min; ESI-MS(+) m/z [M+2H]²⁺: 976.2.

### Preparation of Compound 1561

Compound 1561 was prepared on a 50 μmol scale. The yield of the product was 19.2 mg, and its estimated purity by LCMS analysis was 98.8%. Analysis condition A: Retention time=1.56 min; ESI-MS(+) m/z [M+H]⁺: 1772.

### Preparation of Compound 1562

Compound 1562 was prepared on a 50 μmol scale. The yield of the product was 47.1 mg, and its estimated purity by LCMS analysis was 97.8%. Analysis condition B: Retention time=1.59 min; ESI-MS(+) m/z [M+2H]²⁺: 991.

### Preparation of Compound 1563

Compound 1563 was prepared on a 50 μmol scale. The yield of the product was 28.5 mg, and its estimated purity by LCMS analysis was 98.9%. Analysis condition B: Retention time=1.53 min; ESI-MS(+) m/z [M+3H]³⁺: 636.1.

### Preparation of Compound 1564

Compound 1564 was prepared on a 50 μmol scale. The yield of the product was 36.7 mg, and its estimated purity by LCMS analysis was 97.9%. Analysis condition B: Retention time=1.48, 1.53 min; ESI-MS(+) m/z [M+2H]²⁺: 954.18, 954.14.

### Preparation of Compound 1565

Compound 1565 was prepared on a 50 μmol scale. The yield of the product was 38.1 mg, and its estimated purity by LCMS analysis was 100%. Analysis condition B: Retention time=1.48 min; ESI-MS(+) m/z [M+3H]³⁺: 645.5.

### Preparation of Compound 1566

Compound 1566 was prepared on a 50 μmol scale. The yield of the product was 22.6 mg, and its estimated purity by LCMS analysis was 92.3%. Analysis condition A: Retention time=1.52 min; ESI-MS(+) m/z [M+H]⁺: 1932.1.

### Preparation of Compound 1567

Compound 1567 was prepared on a 50 μmol scale. The yield of the product was 30.7 mg, and its estimated purity by LCMS analysis was 98.6%. Analysis condition B: Retention time=1.6 min; ESI-MS(+) m/z [M+2H]²⁺: 1004.9.

### Preparation of Compound 1568

Compound 1568 was prepared on a 50 μmol scale. The yield of the product was 34.1 mg, and its estimated purity by LCMS analysis was 97.8%. Analysis condition B: Retention time=1.62 min; ESI-MS(+) m/z [M+2H]²⁺: 1011.1.

### Preparation of Compound 1569

Compound 1569 was prepared on a 50 μmol scale. The yield of the product was 51.9 mg, and its estimated purity by LCMS analysis was 95.1%. Analysis condition A: Retention time=1.27 min; ESI-MS(+) m/z [M+2H]²⁺: 1034.2.

### Preparation of Compound 1570

Compound 1570 was prepared on a 50 μmol scale. The yield of the product was 60.4 mg, and its estimated purity by LCMS analysis was 86.7%. Analysis condition B: Retention time=1.6 min; ESI-MS(+) m/z [M+2H]²⁺: 990.

### Preparation of Compound 157

Compound 1571 was prepared on a 50 μmol scale. The yield of the product was 31.9 mg, and its estimated purity by LCMS analysis was 98.2%. Analysis condition A: Retention time=1.46 min; ESI-MS(+) m/z [M+H]⁺: 1952.6.

### Preparation of Compound 1572

Compound 1572 was prepared on a 50 μmol scale. The yield of the product was 27.2 mg, and its estimated purity by LCMS analysis was 96.3%. Analysis condition A: Retention time=1.49 min; ESI-MS(+) m/z [M+2H]²⁺: 983.6.

### Preparation of Compound 1573

Compound 1573 was prepared on a 50 μmol scale. The yield of the product was 18.1 mg, and its estimated purity by LCMS analysis was 100%. Analysis condition A: Retention time=1.38 min; ESI-MS(+) m/z [M+2H]²⁺: 1019.2.

### Preparation of Compound 1574

Compound 1574 was prepared on a 50 μmol scale. The yield of the product was 31.6 mg, and its estimated purity by LCMS analysis was 90.6%. Analysis condition B: Retention time=1.58 min; ESI-MS(+) m/z [M+2H]²⁺: 1006.1.

### Preparation of Compound 1575

Compound 1575 was prepared on a 50 μmol scale. The yield of the product was 62 mg, and its estimated purity by LCMS analysis was 94%. Analysis condition B: Retention time=1.48 min; ESI-MS(+) m/z [M+H]⁺: 1951.

### Preparation of Compound 1576

Compound 1576 was prepared on a 50 μmol scale. The yield of the product was 41.1 mg, and its estimated purity by LCMS analysis was 96.5%. Analysis condition A: Retention time=1.53 min; ESI-MS(+) m/z [M+2H]²⁺: 909.

### Preparation of Compound 1577

Compound 1577 was prepared on a 50 μmol scale. The yield of the product was 65.2 mg, and its estimated purity by LCMS analysis was 98.5%. Analysis condition B: Retention time=1.52 min; ESI-MS(+) m/z [M+2H]²⁺: 1013.1.

### Preparation of Compound 1578

Compound 1578 was prepared on a 50 μmol scale. The yield of the product was 22.6 mg, and its estimated purity by LCMS analysis was 100%. Analysis condition A: Retention time=1.45 min; ESI-MS(+) m/z [M+H]⁺: 1983.5.

### Preparation of Compound 1579

Compound 1579 was prepared on a 50 μmol scale. The yield of the product was 26.5 mg, and its estimated purity by LCMS analysis was 97.8%. Analysis condition A: Retention time=1.32 min; ESI-MS(+) m/z [M+H]⁺: 1925.

### Preparation of Compound 1580

Compound 1580 was prepared on a 50 μmol scale. The yield of the product was 25.6 mg, and its estimated purity by LCMS analysis was 100%. Analysis condition B: Retention time=1.63 min; ESI-MS(+) m/z [M+2H]²⁺: 978.1.

### Preparation of Compound 1581

Compound 1581 was prepared on a 50 μmol scale. The yield of the product was 30.2 mg, and its estimated purity by LCMS analysis was 99.4%. Analysis condition B: Retention time=1.55 min; ESI-MS(+) m/z [M+2H]²⁺: 942.1.

### Preparation of Compound 1582

Compound 1582 was prepared on a 50 μmol scale. The yield of the product was 8.8 mg, and its estimated purity by LCMS analysis was 99%. Analysis condition A: Retention time=1.46 min; ESI-MS(+) m/z [M+2H]²⁺: 910.1.

### Preparation of Compound 1583

Compound 1583 was prepared on a 50 μmol scale. The yield of the product was 57 mg, and its estimated purity by LCMS analysis was 97.8%. Analysis condition A: Retention time=1.32 min; ESI-MS(+) m/z [M+2H]²⁺: 942.1.

### Preparation of Compound 1584

Compound 1584 was prepared on a 50 μmol scale. The yield of the product was 11 mg, and its estimated purity by LCMS analysis was 100%. Analysis condition A: Retention time=1.59 min; ESI-MS(+) m/z [M+H]⁺: 1747.

### Preparation of Compound 1585

Compound 1585 was prepared on a 50 μmol scale. The yield of the product was 38.7 mg, and its estimated purity by LCMS analysis was 98.3%. Analysis condition B: Retention time=2.46 min; ESI-MS(+) m/z [M+2H]²⁺: 1031.9.

### Preparation of Compound 1586

Compound 1586 was prepared on a 50 μmol scale. The yield of the product was 20.1 mg, and its estimated purity by LCMS analysis was 92.7%. Analysis condition B: Retention time=2.2 min; ESI-MS(+) m/z [M+H]⁺: 1959.1.

### Preparation of Compound 1587

Compound 1587 was prepared on a 50 μmol scale. The yield of the product was 41.7 mg, and its estimated purity by LCMS analysis was 98.2%. Analysis condition A: Retention time=1.96 min; ESI-MS(+) m/z [M+2H]²⁺: 1046.9.

### Preparation of Compound 1588

Compound 1588 was prepared on a 50 μmol scale. The yield of the product was 50.2 mg, and its estimated purity by LCMS analysis was 97.2%. Analysis condition A: Retention time=1.84 min; ESI-MS(+) m/z [M+2H]²⁺: 1011.1.

### Preparation of Compound 1589

Compound 1589 was prepared on a 50 μmol scale. The yield of the product was 1.9 mg, and its estimated purity by LCMS analysis was 75.4%. Analysis condition A: Retention time=1.56 min; ESI-MS(+) m/z [M+2H]²⁺: 963.

### Preparation of Compound 1590

Compound 1590 was prepared on a 50 μmol scale. The yield of the product was 10.4 mg, and its estimated purity by LCMS analysis was 92.1%. Analysis condition A: Retention time=1.55 min; ESI-MS(+) m/z [M+2H]²⁺: 931.9.

### Preparation of Compound 1591

Compound 1591 was prepared on a 50 μmol scale. The yield of the product was 27.4 mg, and its estimated purity by LCMS analysis was 96.2%. Analysis condition A: Retention time=1.53 min; ESI-MS(+) m/z [M+2H]²⁺: 975.1.

### Preparation of Compound 1592

Compound 1592 was prepared on a 0 μmol scale. The yield of the product was 24 mg, and its estimated purity by LCMS analysis was 97.6%. Analysis condition A: Retention time=1.52 min; ESI-MS(+) m/z [M+2H]²⁺: 975.

### Preparation of Compound 1593

Compound 1593 was prepared on a 50 μmol scale. The yield of the product was 28.9 mg, and its estimated purity by LCMS analysis was 96.4%. Analysis condition A: Retention time=1.59 min; ESI-MS(+) m/z [M+2H]²⁺: 975.3.

### Preparation of Compound 1594

Compound 1594 was prepare on a 50 μmol scale. The yield of the product was 4.5 mg, and its estimated purity by LCMS analysis was 96.2%. Analysis condition B: Retention time=1.77 min; ESI-MS(+) m/z [M+2H]²⁺: 943.1.

### Preparation of Compound 1595

Compound 1595 was prepared on a 50 μmol scale. The yield of the product was 22.5 mg, and its estimated purity by LCMS analysis was 94%. Analysis condition B: Retention time=1.91 min; ESI-MS(+) m/z [M+H]⁺: 1854.1.

### Preparation of Compound 1596

Compound 1596 was prepared on a 50 μmol scale. The yield of the product was 22.1 mg, and its estimated purity by LCMS analysis was 98%. Analysis condition B: Retention time=1.87 min; ESI-MS(+) m/z [M+2H]²⁺: 949.1.

### Preparation of Compound 1597

Compound 1597 was prepared on a 50 μmol scale. The yield of the product was 11.4 mg, and its estimated purity by LCMS analysis was 92.7%. Analysis condition B: Retention time=1.78 min; ESI-MS(+) m/z [M+H]⁺: 1791.1.

### Preparation of Compound 1598

Compound 1598 was prepared on a 50 μmol scale. The yield of the product was 11.3 mg, and its estimated purity by LCMS analysis was 95.9%. Analysis condition A: Retention time=1.87 min; ESI-MS(+) m/z [M+2H]²⁺: 859.9.

### Preparation of Compound 1599

Compound 1599 was prepared on a 50 μmol scale. The yield of the product was 3.5 mg, and its estimated purity by LCMS analysis was 96.3%. Analysis condition A: Retention time=1.6 min; ESI-MS(+) m/z [M+H]⁺: 1926.9.

### Preparation of Compound 1600

Compound 1600 was prepared on a 50 μmol scale. The yield of the product was 23.6 mg, and its estimated purity by LCMS analysis was 100%. Analysis condition A: Retention time=1.64 min; ESI-MS(+) m/z [M+H]⁺: 1853.

### Preparation of Compound 1601

Compound 1601 was prepared on a 50 μmol scale. The yield of the product was 26.2 mg, and its estimated purity by LCMS analysis was 98.9%. Analysis condition B: Retention time=1.84 min; ESI-MS(+) m/z [M+2H]²⁺: 985.1.

### Preparation of Compound 1602

Compound 1602 was prepared on a 50 μmol scale. The yield of the product was 34.2 mg, and its estimated purity by LCMS analysis was 92.1%. Analysis condition A: Retention time=1.47 min; ESI-MS(+) m/z [M+2H]²⁺: 1006.1.

### Preparation of Compound 1603

Compound 1603 was prepared on a 50 μmol scale. The yield of the product was 29.5 mg, and its estimated purity by LCMS analysis was 97.7%. Analysis condition B: Retention time=1.88 min; ESI-MS(+) m/z [M+H]⁺: 1982.1.

### Preparation of Compound 1604

Compound 1604 was prepared on a 50 μmol scale. The yield of the product was 32.6 mg, and its estimated purity by LCMS analysis was 100%. Analysis condition A: Retention time=1.49 min; ESI-MS(+) m/z [M+2H]²⁺: 1013.1.

### Preparation of Compound 1605

Compound 1605 was prepared on a 50 μmol scale. The yield of the product was 47.7 mg, and its estimated purity by LCMS analysis was 88.7%. Analysis condition A: Retention time=1.44 min; ESI-MS(+) m/z [M+2H]²⁺: 990.2.

### Preparation of Compound 1606

Compound 1606 was prepared on a 50 μmol scale. The yield of the product was 6.5 mg, and its estimated purity by LCMS analysis was 94%. Analysis condition B: Retention time=1.78 min; ESI-MS(+) m/z [M+2H]²⁺: 985.

### Preparation of Compound 1607

Compound 1607 was prepared on a 50 μmol scale. The yield of the product was 13 mg, and its estimated purity by LCMS analysis was 92.8%. Analysis condition A: Retention time=1.56 min; ESI-MS(+) m/z [M+H]⁺: 1937.8.

### Preparation of Compound 1608

Compound 1608 was prepared on a 50 μmol scale. The yield of the product was 10 mg, and its estimated purity by LCMS analysis was 96.8%. Analysis condition A: Retention time=1.55 min; ESI-MS(+) m/z [M+2H]²⁺: 936.9.

### Preparation of Compound 1609

Compound 1609 was prepared on a 50 μmol scale. The yield of the product was 11.7 mg, and its estimated purity by LCMS analysis was 93.4%. Analysis condition A: Retention time=1.66 min; ESI-MS(+) m/z [M+2H]²⁺: 899.9.

### Preparation of Compound 1610

Compound 1610 was prepared on a 50 μmol scale. The yield of the product was 18.1 mg, and its estimated purity by LCMS analysis was 87.3%. Analysis condition B: Retention time=1.68 min; ESI-MS(+) m/z [M+2H]²⁺: 947.9.

### Preparation of Compound 1611

Compound 1611 was prepared on a 50 μmol scale. The yield of the product was 3.5 mg, and its estimated purity by LCMS analysis was 86.5%. Analysis condition A: Retention time=1.69 min; ESI-MS(+) m/z [M+3H]³⁺: 640.4.

### Preparation of Compound 1612

Compound 1612 was prepared on a 50 μmol scale. The yield of the product was 3.7 mg, and its estimated purity by LCMS analysis was 100%. Analysis condition A: Retention time=1.7 min; ESI-MS(+) m/z [M+2H]²⁺: 1962.2.

### Preparation of Compound 1613

Compound 1613 was prepared on a 50 μmol scale. The yield of the product was 16.9 mg, and its estimated purity by LCMS analysis was 98.4%. Analysis condition A: Retention time=1.82 min; ESI-MS(+) m/z [M+2H]²⁺: 959.

### Preparation of Compound 1614

Compound 1614 was prepared on a 50 μmol scale. The yield of the product was 20.8 mg, and its estimated purity by LCMS analysis was 99.1%. Analysis condition B: Retention time=1.95 min; ESI-MS(+) m/z [M+2H]²⁺: 1010.

### Preparation of Compound 1615

Compound 1615 was prepared on a 50 μmol scale. The yield of the product was 10.7 mg, and its estimated purity by LCMS analysis was 100%. Analysis condition A: Retention time=1.82 min; ESI-MS(+) m/z [M+H]⁺: 1913.3.

### Preparation of Compound 1616

Compound 1616 was prepared on a 50 μmol scale. The yield of the product was 9.8 mg, and its estimated purity by LCMS analysis was 98.4%. Analysis condition A: Retention time=1.96 min; ESI-MS(+) m/z [M+H]⁺: 1841.3.

### Preparation of Compound 1617

Compound 1617 was prepared on a 50 μmol scale. The yield of the product was 23.2 mg, and its estimated purity by LCMS analysis was 95.6%. Analysis condition B: Retention time=1.88 min; ESI-MS(+) m/z [M+2H]²⁺: 988.1.

### Preparation of Compound 1618

Compound 1618 was prepared on a 50 μmol scale. The yield of the product was 16.9 mg, and its estimated purity by LCMS analysis was 97.1%. Analysis condition B: Retention time=2.04 min; ESI-MS(+) m/z [M+H]⁺: 1876.2.

### Preparation of Compound 1619

Compound 1619 was prepared on a 50 μmol scale. The yield of the product was 27.6 mg, and its estimated purity by LCMS analysis was 95.3%. Analysis condition A: Retention time=1.56 min; ESI-MS(+) m/z [M+2H]²⁺: 990.3.

### Preparation of Compound 1620

Compound 1620 was prepared on a 50 μmol scale. The yield of the product was 226 mg, and its estimated purity by LCMS analysis was 90.6%. Analysis condition B: Retention time=1.89 min; ESI-MS(+) m/z [M+2H]²⁺: 937.1.

### Preparation of Compound 1621

Compound 1621 was prepared on a 50 μmol scale. The yield of the product was 19.6 mg, and its estimated purity by LCMS analysis was 97.5%. Analysis condition A: Retention time=1.82 min; ESI-MS(+) m/z [M+H]⁺: 1801.3.

### Preparation of Compound 1622

Compound 1622 was prepared on a 50 μmol scale. The yield of the product was 35.1 mg, and its estimated purity by LCMS analysis was 95.1%. Analysis condition A: Retention time=1.63 min; ESI-MS(+) m/z [M+H]⁺: 1935.2.

### Preparation of Compound 1623

Compound 1623 was prepared on a 50 μmol scale. The yield of the product was 22.7 mg, and its estimated purity by LCMS analysis was 95.7%. Analysis condition A: Retention time=1.5 min; ESI-MS(+) m/z [M+2H]²⁺: 1003.3.

### Preparation of Compound 1624

Compound 1624 was prepared on a 50 μmol scale. The yield of the product was 24 mg, and its estimated purity by LCMS analysis was 96.7%. Analysis condition B: Retention time=1.74 min; ESI-MS(+) m/z [M+H]⁺: 1899.9.

### Preparation of Compound 1625

Compound 1625 was prepared on a 50 μmol scale. The yield of the product was 16.7 mg, and its estimated purity by LCMS analysis was 97.4%. Analysis condition B: Retention time=1.6 min; ESI-MS(+) m/z [M+H]⁺: 1827.3.

### Preparation of Compound 1626

Compound 1626 was prepared on a 50 μmol scale. The yield of the product was 26.3 mg, and its estimated purity by LCMS analysis was 97.8%. Analysis condition A: Retention time=1.76 min; ESI-MS(+) m/z [M+2H]²⁺: 981.1.

### Preparation of Compound 1627

Compound 1627 was prepared on a 50 μmol scale. The yield of the product was 25.3 mg, and its estimated purity by LCMS analysis was 94.7%. Analysis condition A: Retention time=1.57 min; ESI-MS(+) m/z [M+H]⁺: 1935.

### Preparation of Compound 1628

Compound 1628 was prepared on a 50 μmol scale. The yield of the product was 34 mg, and its estimated purity by LCMS analysis was 92.5%. Analysis condition A: Retention time=1.56 min; ESI-MS(+) m/z [M+H]⁺: 1908.2.

### Preparation of Compound 1629

Compound 1629 was prepared on a 50 μmol scale. The yield of the product was 3.5 mg, and its estimated purity by LCMS analysis was 96.2%. Analysis condition A: Retention time=1.67 min; ESI-MS(+) m/z [M+H]⁺: 1890.9.

### Preparation of Compound 1630

Compound 1630 was prepared on a 50 μmol scale. The yield of the product was 6.3 mg, and its estimated purity by LCMS analysis was 98.8%. Analysis condition B: Retention time=1.96 min; ESI-MS(+) m/z [M+H]⁺: 1895.1.

### Preparation of Compound 1631

Compound 1631 was prepared on a 50 μmol scale. The yield of the product was 19.3 mg, and its estimated purity by LCMS analysis was 90.8%. Analysis condition A: Retention time=1.56 min; ESI-MS(+) m/z [M+H]⁺: 1837.1.

Compound 1632 was prepared on a 50 μmol scale. The yield of the product was 9.1 mg, and its estimated purity by LCMS analysis was 90.8%. Analysis condition B: Retention time=1.64 min; ESI-MS(+) m/z [M+2H]²⁺: 911.

### Preparation of Compound 1633

Compound 1633 was prepared on a 50 μmol scale. The yield of the product was 22 mg, and its estimated purity by LCMS analysis was 93.7%. Analysis condition B: Retention time=1.56 min; ESI-MS(+) m/z [M+H]⁺: 1806.1.

### Preparation of Compound 1634

Compound 1634 was prepared on a 50 μmol scale. The yield of the product was 22.2 mg, and its estimated purity by LCMS analysis was 97.8%. Analysis condition A: Retention time=1.48 min; ESI-MS(+) m/z [M+H]⁺: 1976.9.

### Preparation of Compound 1635

Compound 1635 was prepared on a 50 μmol scale. The yield of the product was 13.3 mg, and its estimated purity by LCMS analysis was 90.1%. Analysis condition A: Retention time=1.69 min; ESI-MS(+) m/z [M+H]⁺: 1961.1.

### Preparation of Compound 1636

Compound 1636 was prepared on a 50 μmol scale. The yield of the product was 19.6 mg, and its estimated purity by LCMS analysis was 92.4%. Analysis condition A: Retention time=1.63 min; ESI-MS(+) m/z [M+2H]²⁺: 1004.

### Preparation of Compound 1637

Compound 1637 was prepared on a 50 μmol scale. The yield of the product was 25.3 mg, and its estimated purity by LCMS analysis was 93.2%. Analysis condition B: Retention time=1.63 min; ESI-MS(+) m/z [M+2H]: 874.2.

### Preparation of Compound 1638

Compound 1638 was prepared on a 50 μmol scale. The yield of the product was 6.5 mg, and its estimated purity by LCMS analysis was 86.6%. Analysis condition A: Retention time=1.67 min; ESI-MS(+) m/z [M+H]⁺: 1868.1.

### Preparation of Compound 1639

Compound 1639 was prepared on a 50 μmol scale. The yield of the product was 6.5 mg, and its estimated purity by LCMS analysis was 92.8%. Analysis condition A: Retention time=1.63 min; ESI-MS(+) m/z [M+2H]²⁺: 956.

### Preparation of Compound 1640

Compound 1640 was prepared on a 50 μmol scale. The yield of the product was 5.5 mg, and its estimated purity by LCMS analysis was 98%. Analysis condition B: Retention time=1.9 min; ESI-MS(+) m/z [M+2H]²⁺: 903.4.

### Preparation of Compound 1641

Compound 1641 was prepared on a 50 μmol scale. The yield of the product was 6.2 mg, and its estimated purity by LCMS analysis was 87.3%. Analysis condition A: Retention time=1.62 min; ESI-MS(+) m/z [M+H]⁺: 1804.9.

### Preparation of Compound 1642

Compound 1642 was prepared on a 50 μmol scale. The yield of the product was 9.5 mg, and its estimated purity by LCMS analysis was 92.7%. Analysis condition B: Retention time=1.72 min; ESI-MS(+) m/z [M+2H]²⁺: 956.

### Preparation of Compound 1643

Compound 1643 was prepared on a 50 μmol scale. The yield of the product was 8.8 mg, and its estimated purity by LCMS analysis was 94.4%. Analysis condition B: Retention time=1.82 min; ESI-MS(+) m/z [M+2H]²⁺: 978.2.

### Preparation of Compound 1644

Compound 1644 was prepared on a 50 μmol scale. The yield of the product was 7.1 mg, and its estimated purity by LCMS analysis was 86%. Analysis condition A: Retention time=1.49 min; ESI-MS(+) m/z [M+2H]²⁺: 925.

### Preparation of Compound 1645

Compound 1645 was prepared on a 50 μmol scale. The yield of the product was 15.2 mg, and its estimated purity by LCMS analysis was 100%. Analysis condition A: Retention time=1.7, 1.78 min; ESI-MS(+) m/z [M+2H]²⁺: 1058.04, 1057.94.

### Preparation of Compound 1646

Compound 1646 was prepared on a 50 μmol scale. The yield of the product was 22 mg, and its estimated purity by LCMS analysis was 96.7%. Analysis condition 3: Retention time=1.53 min; ESI-MS(+) m/z [M+2H]²⁺: 1038.

### Preparation of Compound 1647

Compound 1647 was prepared on a 50 μmol scale. The yield of the product was 37.7 mg, and its estimated purity by LCMS analysis was 98.6%. Analysis condition: Retention time=1.77 min; ESI-MS(+) m/z [M+2H]²⁺: 1038.1.

### Preparation of Compound 1648

Compound 1648 was prepared on a 50 μmol scale. The yield of the product was 30.8 mg, and its estimated purity by LCMS analysis was 99.1%. Analysis condition: Retention time=1.82 min; ESI-MS(+) m/z [M+2H]²⁺: 1038.1.

### Preparation of Compound 1649

Compound 1649 was prepared on a 50 μmol scale. The yield of the product was 11.9 mg, and its estimated purity by LCMS analysis was 95.9%. Analysis condition 4: Retention time=1.75 min; ESI-MS(+) m/z [M+2H]²⁺: 1045.

### Preparation of Compound 1650

Compound 1650 was prepared on a 50 μmol scale. The yield of the product was 13.7 mg, and its estimated purity by LCMS analysis was 96.8%. Analysis condition B: Retention time=1.77 min; ESI-MS(+) m/z [M+2H]²⁺: 1031.

### Preparation of Compound 1651

Compound 1651 was prepared on a 50 μmol scale. The yield of the product was 36.9 mg, and its estimated purity by LCMS analysis was 92.8%. Analysis condition: Retention time=1.52 min; ESI-MS(+) m/z [M+2H]²⁺: 1045.1.

### Preparation of Compound 1652

Compound 1652 was prepared on a 50 μmol scale. The yield of the product was 5 mg, and its estimated purity by LCMS analysis was 100%. Analysis condition: Retention time=1.5 min; ESI-MS(+) m/z [M+2H]²⁺: 1105.4.

### Preparation of Compound 1653

Compound 1653 was prepared on a 50 μmol scale. The yield of the product was 39.4 mg, and its estimated purity by LCMS analysis was 95%. Analysis condition: Retention time=1.41 min; ESI-MS(+) m/z [M+2H]²⁺: 1105.3.

### Preparation of Compound 1654

Compound 1654 was prepared on a 50 μmol scale. The yield of the product was 27.4 mg, and its estimated purity by LCMS analysis was 95.9%. Analysis condition B: Retention time=1.86 min; ESI-MS(+) m/z [M+2H]²⁺: 1106.2.

### Preparation of Compound 1655

Compound 1655 was prepared on a 50 μmol scale. The yield of the product was 47.7 mg, and its estimated purity by LCMS analysis was 97.7%. Analysis condition: Retention time=1.87 min; ESI-MS(+) m/z [M+2H]²⁺: 1112.1.

### Preparation of Compound 1656

Compound 1656 was prepared on a 50 μmol scale. The yield of the product was 35.9 mg, and its estimated purity by LCMS analysis was 94%. Analysis condition: Retention time=1.43 min; ESI-MS(+) m/z [M+2H]²⁺: 1099.

### Preparation of Compound 1657

Compound 1657 was prepared on a 50 μmol scale. The yield of the product was 24.3 mg, and its estimated purity by LCMS analysis was 96%. Analysis condition B: Retention time=1.85 min; ESI-MS(+) m/z [M+2H]²⁺: 1113.1.

### Preparation of Compound 1658

Compound 1658 was prepared on a 50 μmol scale. The yield of the product was 34.7 mg, and its estimated purity by LCMS analysis was 96%. Analysis condition B: Retention time=1.82 min; ESI-MS(+) m/z [M+2H]²⁺: 1069.1.

### Preparation of Compound 1659

Compound 1659 was prepared on a 50 μmol scale. The yield of the product was 54.8 mg, and its estimated purity by LCMS analysis was 97.6%. Analysis condition B: Retention time=1.76 min; ESI-MS(+) m/z [M+2H]²⁺: 1076.

### Preparation of Compound 1660

Compound 1660 was prepared on a 50 μmol scale. The yield of the product was 51.5 mg, and its estimated purity by LCMS analysis was 96%. Analysis condition B: Retention time=1.76 min; ESI-MS(+) m/z [M+2H]²⁺: 1062.2.

### Preparation of Compound 1661

Compound 1661 was prepared on a 50 μmol scale. The yield of the product was 24.7 mg, and its estimated purity by LCMS analysis was 97%. Analysis condition B: Retention time=1.73 min; ESI-MS(+) m/z [M+2H]²⁺: 1076.

### Preparation of Compound 1662

Compound 1662 was prepared on a 50 μmol scale. The yield of the product was 6.2 mg, and its estimated purity by LCMS analysis was 96%. Analysis condition B: Retention time=1.72 min; ESI-MS(+) m/z [M+3H]³⁺: 713.2.

### Preparation of Compound 1663

Compound 1663 was prepared on a 50 μmol scale. The yield of the product was 40.9 mg, and its estimated purity by LCMS analysis was 95.1%. Analysis condition B: Retention time=1.73 min; ESI-MS(+) m/z [M+2H]²⁺: 1069.

### Preparation of Compound 1664

Compound 1664 was prepared on a 50 μmol scale. The yield of the product was 35.7 mg, and its estimated purity by LCMS analysis was 90.6%. Analysis condition B: Retention time=1.9 min; ESI-MS(+) m/z [M+2H]²⁺: 1052.

### Preparation of Compound 1665

Compound 1665 was prepared on a 50 μmol scale. The yield of the product was 38.2 mg, and its estimated purity by LCMS analysis was 98.1%. Analysis condition B: Retention time=1.87 min; ESI-MS(+) m/z [M+2H]²⁺: 1059.2.

### Preparation of Compound 1666

Compound 1666 was prepared on a 50 μmol scale. The yield of the product was 54.6 mg, and its estimated purity by LCMS analysis was 97.1%. Analysis condition B: Retention time=1.84 min; ESI-MS(+) m/z [M+2H]²⁺: 1045.3.

### Preparation of Compound 1667

Compound 1667 was prepared on a 50 μmol scale. The yield of the product was 40 mg, and its estimated purity by LCMS analysis was 95.7%. Analysis condition B: Retention time=1.8 min; ESI-MS(+) m/z [M+2H]²⁺: 1059.1.

### Preparation of Compound 1668

Compound 1668 was prepared on a 50 μmol scale. The yield of the product was 25.8 mg, and its estimated purity by LCMS analysis was 98.1%. Analysis condition B: Retention time=1.78 min; ESI-MS(+) m/z [M+2H]²⁺: 1052.3.

### Preparation of Compound 1669

Compound 1669 was prepared on a 50 μmol scale. The yield of the product was 33.9 mg, and its estimated purity by LCMS analysis was 98.5%. Analysis condition B: Retention time=1.86 min; ESI-MS(+) m/z [M+2H]²⁺: 1044.9.

### Preparation of Compound 1670

Compound 1670 was prepared on a 50 μmol scale. The yield of the product was 29.6 mg, and its estimated purity by LCMS analysis was 90.1%. Analysis condition 1.87: Retention time=1.87 min; ESI-MS(+) m/z [M+2H]²⁺: 1045.9.

### Preparation of Compound 1671

Compound 1671 was prepared on a 50 μmol scale. The yield of the product was 27 mg, and its estimated purity by LCMS analysis was 90.9%. Analysis condition A: Retention time=1.47 min; ESI-MS(+) m/z [M+2H]²⁺: 1052.1.

### Preparation of Compound 1672

Compound 1672 was prepared on a 50 μmol scale. The yield of the product was 21.9 mg, and its estimated purity by LCMS analysis was 100%. Analysis condition A: Retention time=1.53 min; ESI-MS(+) m/z [M+2H]²⁺: 1039.1.

### Preparation of Compound 1673

Compound 1673 was prepared on a 50 μmol scale. The yield of the product was 14.8 mg, and its estimated purity by LCMS analysis was 98.5%. Analysis condition B: Retention time=1.91 min; ESI-MS(+) m/z [M+2H]²⁺: 1053.

### Preparation of Compound 1674

Compound 1674 was prepared on a 50 μmol scale. The yield of the product was 18 mg, and its estimated purity by LCMS analysis was 97.6%. Analysis condition B: Retention time=1.74 min; ESI-MS(+) m/z [M+2H]²⁺: 1045.2.

### Preparation of Compound 1675

Compound 1675 was prepared on a 50 μmol scale. The yield of the product was 42 mg, and its estimated purity by LCMS analysis was 96.6%. Analysis condition B: Retention time=1.96 min; ESI-MS(+) m/z [M+3H]³⁺: 702.3.

### Preparation of Compound 1676

Compound 1676 was prepared on a 50 μmol scale. The yield of the product was 49.9 mg, and its estimated purity by LCMS analysis was 93.5%. Analysis condition A: Retention time=1.3, 1.34 min; ESI-MS(+) m/z [M+2H]²⁺: 1052.97, 1052.97.

### Preparation of Compound 1677

Compound 1677 was prepared on a 50 μmol scale. The yield of the product was 47.9 mg, and its estimated purity by LCMS analysis was 99%. Analysis condition: B Retention time=1.95 min; ESI-MS(+) m/z [M+2H]²⁺: 1059.2.

### Preparation of Compound 1678

Compound 1678 was prepared on a 50 μmol scale. The yield of the product was 28.7 mg, and its estimated purity by LCMS analysis was 92.5%. Analysis condition B: Retention time=2.04 min; ESI-MS(+) m/z [M+2H]²⁺: 1046.1.

### Preparation of Compound 1679

Compound 1679 was prepared on a 50 μmol scale. The yield of the product was 35.8 mg, and its estimated purity by LCMS analysis was 92.4%. Analysis condition A: Retention time=1.35 min; ESI-MS(+) m/z [M+2H]²⁺: 1059.9.

### Preparation of Compound 1680

Compound 1680 was prepared on a 50 μmol scale. The yield of the product was 22.4 mg, and its estimated purity by LCMS analysis was 98.6%. Analysis condition B: Retention time=1.89 min; ESI-MS(+) m/z [M+2H]²⁺: 1052.2.

### Preparation of Compound 1681

Compound 1681 was prepared on a 50 μmol scale. The yield of the product was 28.4 mg, and its estimated purity by LCMS analysis was 96.4%. Analysis condition B: Retention time=2.19 min; ESI-MS(+) m/z [M+2H]²⁺: 969.1.

### Preparation of Compound 1682

Compound 1682 was prepared on a 50 μmol scale. The yield of the product was 3 mg, and its estimated purity by LCMS analysis was 96.2%. Analysis condition A: Retention time=1.62 min; ESI-MS(+) m/z [M+H]⁺: 1910.1.

### Preparation of Compound 1683

Compound 1683 was prepared on a 50 μmol scale. The yield of the product was 32.5 mg, and its estimated purity by LCMS analysis was 98.8%. Analysis condition 7: Retention time=2.22 min; ESI-MS(+) m/z [M+H]⁺: 1950.2.

### Preparation of Compound 1684

Compound 1684 was prepared on a 50 μmol scale. The yield of the product was 35 mg, and its estimated purity by LCMS analysis was 93.6%. Analysis condition A: Retention time=1.61 min; ESI-MS(+) m/z [M+H]⁺: 1878.1.

### Preparation of Compound 1685

Compound 1685 was prepared on a 50 μmol scale. The yield of the product was 32.3 mg, and its estimated purity by LCMS analysis was 95.7%. Analysis condition A: Retention time=1.69 min; ESI-MS(+) m/z [M+H]⁺: 1849.

### Preparation of Compound 1686

Compound 1686 was prepared on a 50 μmol scale. The yield of the product was 42.5 mg, and its estimated purity by LCMS analysis was 95.1%. Analysis condition A: Retention time=1.65 min; ESI-MS(+) m/z [M+H]⁺: 1878.1.

### Preparation of Compound 1687

Compound 1687 was prepared on a 50 μmol scale. The yield of the product was 42.1 mg, and its estimated purity by LCMS analysis was 97.1%. Analysis condition B: Retention time=1.61 min; ESI-MS(+) m/z [M+H]⁺: 1893.2.

### Preparation of Compound 1688

Compound 1688 was prepared on a 50 μmol scale. The yield of the product was 51.2 mg, and its estimated purity by LCMS analysis was 99.2%. Analysis condition A: Retention time=1.72 min; ESI-MS(+) m/z [M+H]⁺: 1864.

### Preparation of Compound 1689

Compound 1689 was prepared on a 50 μmol scale. The yield of the product was 41.6 mg, and its estimated purity by LCMS analysis was 100%. Analysis condition A: Retention time=1.86 min; ESI-MS(+) m/z [M+H]⁺: 1917.1.

### Preparation of Compound 1690

Compound 1690 was prepared on a 50 μmol scale. The yield of the product was 51.4 mg, and its estimated purity by LCMS analysis was 100%. Analysis condition A: Retention time=1.91 min; ESI-MS(+) m/z [M+H]⁺: 1888.

### Preparation of Compound 1691

Compound 1691 was prepared on a 50 μmol scale. The yield of the product was 49 mg, and its estimated purity by LCMS analysis was 93.3%. Analysis condition B: Retention time=1.75 min; ESI-MS(+) m/z [M+H]⁺: 1917.

### Preparation of Compound 1692

Compound 1692 was prepared on a 50 μmol scale. The yield of the product was 35.6 mg, and its estimated purity by LCMS analysis was 100%. Analysis condition A: Retention time=1.97 min; ESI-MS(+) m/z [M+H]⁺: 1887.3.

### Preparation of Compound 1693

Compound 1693 was prepared on a 50 μmol scale. The yield of the product was 8.8 mg, and its estimated purity by LCMS analysis was 94.7%. Analysis condition B: Retention time=1.86 min; ESI-MS(+) m/z [M+2H]²¹: 967.3.

### Preparation of Compound 1694

Compound 1694 was prepared on a 50 μmol scale. The yield of the product was 42.8 mg, and its estimated purity by LCMS analysis was 100%. Analysis condition A: Retention time=1.98 min; ESI-MS(+) m/z [M+H]⁺: 1904.3.

### Preparation of Compound 1695

Compound 1695 was prepared on a 50 μmol scale. The yield of the product was 24.3 mg, and its estimated purity by LCMS analysis was 100%. Analysis condition A: Retention time=1.69 min; ESI-MS(+) m/z [M+H]⁺: 1945.3.

### Preparation of Compound 1696

Compound 1696 was prepared on a 50 μmol scale. The yield of the product was 18.7 mg, and its estimated purity by LCMS analysis was 96.2%. Analysis condition A: Retention time=1.81 min; ESI-MS(+) m/z [M+H]⁺: 1916.

### Preparation of Compound 1697

Compound 1697 was prepared on a 50 μmol scale. The yield of the product was 21.2 mg, and its estimated purity by LCMS analysis was 100%. Analysis condition A: Retention time=1.87 min; ESI-MS(+) m/z [M+H]⁺: 1944.3.

### Preparation of Compound 1698

Compound 1698 was prepared on a 50 μmol scale. The yield of the product was 9.1 mg, and its estimated purity by LCMS analysis was 91.7%. Analysis condition A: Retention time=1.89 min; ESI-MS(+) m/z [M+H]⁺: 1916.

### Preparation of Compound 1699

Compound 1699 was prepared on a 50 μmol scale. The yield of the product was 4.5 mg, and its estimated purity by LCMS analysis was 80.4%. Analysis condition A: Retention time=1.81 min; ESI-MS(+) m/z [M+H]⁺: 1961.

### Preparation of Compound 1700

Compound 1700 was prepared on a 50 μmol scale. The yield of the product was 20.5 mg, and its estimated purity by LCMS analysis was 84%. Analysis condition B: Retention time=1.85 min; ESI-MS(+) m/z [M+H]⁺: 1931.9.

### Preparation of Compound 1701

Compound 1701 was prepared on a 50 μmol scale. The yield of the product was 45.7 mg, and its estimated purity by LCMS analysis was 93.9%. Analysis condition A: Retention time=1.75 min; ESI-MS(+) m/z [M+H]⁺: 1961.1.

### Preparation of Compound 1702

Compound 1702 was prepared on a 50 μmol scale. The yield of the product was 38.7 mg, and its estimated purity by LCMS analysis was 97.4%. Analysis condition B: Retention time=1.7 min; ESI-MS(+) m/z [M+H]⁺: 1929.6.

### Preparation of Compound 1703

Compound 1703 was prepared on a 50 μmol scale. The yield of the product was 24.5 mg, and its estimated purity by LCMS analysis was 100%. Analysis condition A: Retention time=1.7 min; ESI-MS(+) m/z [M+H]⁺: 1961.1.

### Preparation of Compound 1704

Compound 1704 was prepared on a 50 μmol scale. The yield of the product was 51.5 mg, and its estimated purity by LCMS analysis was 97.4%. Analysis condition A: Retention time=1.86 min; ESI-MS(+) m/z [M+H]⁺: 1932.3.

### Preparation of Compound 1705

Compound 1705 was prepared on a 50 μmol scale. The yield of the product was 37.7 mg, and its estimated purity by LCMS analysis was 100%. Analysis condition A: Retention time=1.7 min; ESI-MS(+) m/z [M+H]⁺: 1976.8.

### Preparation of Compound 1706

Compound 1706 was prepared on a 50 μmol scale. The yield of the product was 32.7 mg, and its estimated purity by LCMS analysis was 100%. Analysis condition A: Retention time=1.79 min; ESI-MS(+) m/z [M+H]⁺: 1948.

### Preparation of Compound 1707

Compound 1707 was prepared on a 50 μmol scale. The yield of the product was 48.6 mg, and its estimated purity by LCMS analysis was 91%. Analysis condition A: Retention time=1.74 min; ESI-MS(+) m/z [M+H]⁺: 1989.

### Preparation of Compound 1708

Compound 1708 was prepared on a 50 μmol scale. The yield of the product was 65.2 mg, and its estimated purity by LCMS analysis was 88.3%. Analysis condition A: Retention time=1.62 min; ESI-MS(+) m/z [M+2H]²⁺: 981.

### Preparation of Compound 1709

Compound 1709 was prepared on a 50 μmol scale. The yield of the product was 26 mg, and its estimated purity by LCMS analysis was 95.3%. Analysis condition A: Retention time=1.78 min; ESI-MS(+) m/z [M+H]⁺: 1989.

### Preparation of Compound 1710

Compound 1710 was prepared on a 50 μmol scale. The yield of the product was 36.1 mg, and its estimated purity by LCMS analysis was 97.6%. Analysis condition A: Retention time=1.75 min; ESI-MS(+) m/z [M+H]⁺: 1960.1.

### Preparation of Compound 1711

Compound 1711 was prepared on a 50 μmol scale. The yield of the product was 55.2 mg, and its estimated purity by LCMS analysis was 95.3%. Analysis condition A: Retention time=1.76 min; ESI-MS(+) m/z [M+2H]²⁺: 1003.1.

### Preparation of Compound 1712

Compound 1712 was prepared on a 50 μmol scale. The yield of the product was 33.3 mg, and its estimated purity by LCMS analysis was 97%. Analysis condition A: Retention time=1.77 min; ESI-MS(+) m/z [M+H]⁺: 1976.3.

### Preparation of Compound 1713

Compound 1713 was prepared on a 50 μmol scale. The yield of the product was 16.8 mg, and its estimated purity by LCMS analysis was 85%. Analysis condition B: Retention time=1.67 min; ESI-MS(+) m/z [M+2H]²⁺: 1062.3.

### Preparation of Compound 1714

Compound 1714 was prepared on a 50 μmol scale. The yield of the product was 8.7 mg, and its estimated purity by LCMS analysis was 91.7%. Analysis condition A: Retention time=1.62 min; ESI-MS(+) m/z [M+2H]²⁺: 1026.1.

### Preparation of Compound 1715

Compound 1715 was prepared on a 50 μmol scale. The yield of the product was 8.7 mg, and its estimated purity by LCMS analysis was 97.9%. Analysis condition B: Retention time=1.55 min; ESI-MS(+) m/z [M+2H]²⁺: 1046.5.

### Preparation of Compound 1716

Compound 1716 was prepared on a 50 μmol scale. The yield of the product was 8.7 mg, and its estimated purity by LCMS analysis was 100%. Analysis condition A: Retention time=1.62 min; ESI-MS(+) m/z [M+2H]²⁺: 1040.3.

### Preparation of Compound 1717

Compound 1717 was prepared on a 50 μmol scale. The yield of the product was 8.7 mg, and its estimated purity by LCMS analysis was 96.2%. Analysis condition A: Retention time=1.73 min; ESI-MS(+) m/z [M+2H]²⁺: 1061.

### Preparation of Compound 1718

Compound 1718 was prepared on a 25 μmol scale. The yield of the product was 2.1 mg, and its estimated purity by LCMS analysis was 97.8%. Analysis condition B: Retention time=1.9 min; ESI-MS(+) m/z [M+2H]²⁺: 1102.

### Preparation of Compound 1719

Compound 1719 was prepared on a 25 μmol scale. The yield of the product was 4.2 mg, and its estimated purity by LCMS analysis was 98.7%. Analysis condition B: Retention time=1.87 min; ESI-MS(+) m/z [M+2H]²⁺: 1115.1.

### Preparation of Compound 1720

Compound 1720 was prepared on a 50 μmol scale. The yield of the product was 3.4 mg, and its estimated purity by LCMS analysis was 92.1%. Analysis condition A: Retention time=1.69 min; ESI-MS(+) m/z [M+2H]²⁺: 1108.2.

### Preparation of Compound 1721

Compound 1721 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 38.9 mg, and its estimated purity by LCMS analysis was 100%. Analysis condition A: Retention time=1.66 min; ESI-MS(+) m/z [M+2H]²⁺: 911.

### Preparation of Compound 1722

Compound 1722 was prepared, using Sieber or Rink on a 50 μmol scale, following the general synthetic sequence described for the preparation of Compound 1001 and Compound 1000, composed of the following general procedures: “Symphony X Resin-swelling procedure”, “Symphony X Single-coupling procedure”, “Symphony Single-Coupling Pre-Activation Procedure” was followed with Fmoc-4-Pyr-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-55% B over 20 minutes, then a 5-minute hold at 100% B; Flow: 20 mL/min. Fractions containing the desired product were combined and dried via centrifugal evaporation. The yield of the product was 36.2 mg, and its estimated purity by LCMS analysis was 98.6%. Analysis condition A: Retention time=1.5 min; ESI-MS(+) m/z [M+H]⁺: 1835.

### Preparation of Compound 1723

Compound 1723 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation” for Fmoc-Phe(3-CN)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 4-minute hold at 100% B; Flow: 20 mL/min. Fractions containing the desired product were combined and dried via centrifugal evaporation. The yield of the product was 39.6 mg, and its estimated purity by LCMS analysis was 97.7%. Analysis condition B: Retention time=1.73 min; ESI-MS(+) m/z [M+H]⁺: 1857.2.

### Preparation of Compound 1724

Compound 1724 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-DOPA(Acetonide)-OH (S)-2-((((9H-fluoren-9-yl)methoxy)carbonyl)amino)-3-(3,4-di-tert-butoxyphenyl)propanoic acid, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 20-60% B over 20 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 4.5 mg, and its estimated purity by LCMS analysis was 99.3%. Analysis condition B: Retention time=1.75 min; ESI-MS(+) m/z [M+H]⁺: 1865.2.

### Preparation of Compound 1725

Compound 1725 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 19 minutes, then a 5-minute hold at 100% B; Flow: 20 mL/min. Fractions containing the desired product were combined and dried via centrifugal evaporation. The yield of the product was 14.9 mg, and its estimated purity by LCMS analysis was 98.9%. Analysis condition A: Retention time=1.67 min; ESI-MS(+) m/z [M+2H]²⁺: 919.3.

### Preparation of Compound 1726

Compound 1726 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 13.6 mg, and its estimated purity by LCMS analysis was 97.9%. Analysis condition A: Retention time=1.88 min; ESI-MS(+) m/z [M+H]⁺: 1858.

### Preparation of Compound 1727

Compound 1727 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 23.8 mg, and its estimated purity by LCMS analysis was 100%. Analysis condition A: Retention time=1.66 min; ESI-MS(+) m/z [M+H]⁺: 1847.9.

### Preparation of Compound 1728

Compound 1728 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Phe(3-Me)-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 30-70% B over 20 minutes, then a 4-minute hold at 100% B; Flow: 20 mL/min. Fractions containing the desired product were combined and dried via centrifugal evaporation. The yield of the product was 14.9 mg, and its estimated purity by LCMS analysis was 95%. Analysis condition A: Retention time=1.91 min; ESI-MS(+) m/z [M+H]⁺: 1846.

### Preparation of Compound 1729

Compound 1729 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 41.6 mg, and its estimated purity by LCMS analysis was 100%. Analysis condition B: Retention time=1.68 min; ESI-MS(+) m/z [M+2H]²⁺: 918.2.

### Preparation of Compound 1730

Compound 1730 was prepared, using Sieber or Rink on a 50 μmol scale, following the general synthetic sequence described for the preparation of Compound 1001 and Compound 1000, composed of the following general procedures: “Symphony X Resin-swelling procedure”, “Symphony X Single-coupling procedure”, “Symphony Single-Coupling Pre-Activation Procedure” was followed with Fmoc-4-Pyr-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 31.3 mg, and its estimated purity by LCMS analysis was 100%. Analysis condition A: Retention time=1.4 min; ESI-MS(+) m/z [M+2H]²⁺: 925.3.

### Preparation of Compound 1731

Compound 1731 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Single-Coupling Manual Addition Procedure B” was followed with Fmoc-Phe(4-COOtBu)-OH; “Symphony X Chloroacetic Anhydride coupling procedure”, “Symphony X Final rinse and dry procedure”, “Global Deprotection Method”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 12-52% B over 20 minutes, then a 4-minute hold at 100% B; Flow: 20 mL/min. Fractions containing the desired product were combined and dried via centrifugal evaporation. The yield of the product was 48.1 mg, and its estimated purity by LCMS analysis was 100%. Analysis condition A: Retention time=1.48 min; ESI-MS(+) m/z [M+2H]²⁺: 939.

### Preparation of Compound 1732

Compound 1732 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Phe(3-OMe)-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 23.6 mg, and its estimated purity by LCMS analysis was 97.3%. Analysis condition A: Retention time=1.76 min; ESI-MS(+) m/z [M+H]⁺: 1862.

### Preparation of Compound 1733

Compound 1733 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 20-70% B over 20 minutes, then a 2-minute hold at 100% B; Flow: 40 mL/min. Fractions containing the desired product were combined and dried via centrifugal evaporation. The yield of the product was 22.4 mg, and its estimated purity by LCMS analysis was 92.2%. Analysis condition A: Retention time=1.79 min; ESI-MS(+) m/z [M+2H]²⁺: 903.1.

### Preparation of Compound 1734

Compound 1734 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 4.8 mg, and its estimated purity by LCMS analysis was 95.9%. Analysis condition A: Retention time=1.68 min; ESI-MS(+) m/z [M+H]⁺: 1823.

### Preparation of Compound 1735

Compound 1735 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 19 minutes, then a 5-minute hold at 100% B; Flow: 20 mL/min. Fractions containing the desired product were combined and dried via centrifugal evaporation. The yield of the product was 22.6 mg, and its estimated purity by LCMS analysis was 98.2%. Analysis condition A: Retention time=1.71 min; ESI-MS(+) m/z [M+2H]²⁺: 925.3.

### Preparation of Compound 1736

Compound 1736 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 25 minutes, then a 5-minute hold at 100% B; Flow: 20 mL/min. Fractions containing the desired product were combined and dried via centrifugal evaporation. The yield of the product was 5.1 mg, and its estimated purity by LCMS analysis was 95.3%. Analysis condition A: Retention time=1.37 min; ESI-MS(+) m/z [M+2H]²⁺: 920.2.

### Preparation of Compound 1737

Compound 1737 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Tyr(CH₂COOtBu)-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 20 mg, and its estimated purity by LCMS analysis was 99.4%. Analysis condition B: Retention time=1.61 min; ESI-MS(+) m/z [M+2H]²⁺: 954.2.

### Preparation of Compound 1738

Compound 1738 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Tyr(3-NO₂)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 30-70% B over 20 minutes, then a 5-minute hold at 100% B; Flow: 20 mL/min. Fractions containing the desired product were combined and dried via centrifugal evaporation. The yield of the product was 5 mg, and its estimated purity by LCMS analysis was 97.6%. Analysis condition A: Retention time=1.64 min; ESI-MS(+) m/z [M+2H]²⁺: 947.2.

### Preparation of Compound 1739

Compound 1739 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Phe(3-OMe)-OH; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 5-55% B over 20 minutes, then a 2-minute hold at 100% B; Flow: 45 mL/min. Fractions containing the desired product were combined and dried via centrifugal evaporation. The yield of the product was 26 mg, and its estimated purity by LCMS analysis was 98.4%. Analysis condition B: Retention time=1.84 min; ESI-MS(+) m/z [M+H]⁺: 1839.3.

### Preparation of Compound 1740

Compound 1740 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Phe(4-CONH₂), “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 24.2 mg, and its estimated purity by LCMS analysis was 98.3%. Analysis condition A: Retention time=1.56 min; ESI-MS(+) m/z [M+H]⁺: 1875.2.

### Preparation of Compound 1741

Compound 1741 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-3-Pyr-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 17-57% B over 25 minutes, then a 5-minute hold at 100% B; Flow: 20 mL/min. Fractions containing the desired product were combined and dried via centrifugal evaporation. The yield of the product was 31.4 mg, and its estimated purity by LCMS analysis was 99.4%. Analysis condition A: Retention time=1.43 min; ESI-MS(+) m/z [M+2H]²⁺: 925.1.

### Preparation of Compound 1742

Compound 1742 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 20-60% B over 20 minutes, then a 5-minute hold at 100% B; Flow: 20 mL/min. Fractions containing the desired product were combined and dried via centrifugal evaporation. The yield of the product was 33.7 mg, and its estimated purity by LCMS analysis was 97.9%. Analysis condition A: Retention time=2 min; ESI-MS(+) m/z [M+H]⁺: 1934.3.

### Preparation of Compound 1743

Compound 1743 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-3-Pyr-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 23.5 mg, and its estimated purity by LCMS analysis was 99.4%. Analysis condition B: Retention time=1.57 min; ESI-MS(+) m/z [M+2H]²⁺: 918.2.

### Preparation of Compound 1744

Compound 1744 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 22-62% B over 20 minutes, then a 3-minute hold at 100% B; Flow: 20 mL/min. Fractions containing the desired product were combined and dried via centrifugal evaporation. The yield of the product was 34.4 mg, and its estimated purity by LCMS analysis was 99.3%. Analysis condition A: Retention time=1.55 min; ESI-MS(+) m/z [M+2H]²⁺: 931.1.

### Preparation of Compound 1745

Compound 1745 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Leu-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 20 minutes, then a 5-minute hold at 100% B; Flow: 20 mL/min. Fractions containing the desired product were combined and dried via centrifugal evaporation. The yield of the product was 39.1 mg, and its estimated purity by LCMS analysis was 100%. Analysis condition A: Retention time=1.7 min; ESI-MS(+) m/z [M+2H]²⁺: 945.7.

### Preparation of Compound 1746

Compound 1746 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Single-Coupling Manual Addition Procedure B” was followed with Fmoc-Phe(4-COOtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 12-52% B over 20 minutes, then a 5-minute hold at 100% B; Flow: 20 mL/min. Fractions containing the desired product were combined and dried via centrifugal evaporation. The yield of the product was 26.5 mg, and its estimated purity by LCMS analysis was 100%. Analysis condition A: Retention time=1.21 min; ESI-MS(+) m/z [M+2H]²⁺: 961.

### Preparation of Compound 1747

Compound 1747 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Single-Coupling Manual Addition Procedure B” was followed with Fmoc-Phe(4-COOtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 20 minutes, then a 5-minute hold at 100% B; Flow: 20 mL/min. Fractions containing the desired product were combined and dried via centrifugal evaporation. The yield of the product was 19.8 mg, and its estimated purity by LCMS analysis was 100%. Analysis condition A: Retention time=1.29 min; ESI-MS(+) m/z [M+2H]²⁺: 939.1.

### Preparation of Compound 1748

Compound 1748 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 14.8 mg, and its estimated purity by LCMS analysis was 98%. Analysis condition B: Retention time=1.6 min; ESI-MS(+) m/z [M+2H]²⁺: 932.2.

### Preparation of Compound 1749

Compound 1749 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 17.2 mg, and its estimated purity by LCMS analysis was 100%. Analysis condition A: Retention time=1.36 min; ESI-MS(+) m/z [M+2H]²⁺: 932.4.

### Preparation of Compound 1750

Compound 1750 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Phe(4-CH₂NHBoc)-OH, “Symphony Chloroacetic Anhydride coupling procedure”, “Global Deprotection Method A” and “Cyclization Method”.

The crude material was purified via preparative LC/MS with the following conditions: Column: waters xbridge c-18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-60% B over 20 minutes, then a 5-minute hold at 100% B; Flow: 20 mL/min. Fractions containing the desired product were combined and dried via centrifugal evaporation. The yield of the product was 19.3 mg, and its estimated purity by LCMS analysis was 98.4%. Analysis condition A: Retention time=1.73 min; ESI-MS(+) m/z [M+H]⁺: 1861.1.

### Preparation of Compound 1751

Compound 1751 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 18-58% B over 20 minutes, then a 3-minute hold at 100% B; Flow: 20 mL/min. Fractions containing the desired product were combined and dried via centrifugal evaporation. The yield of the product was 17.3 mg, and its estimated purity by LCMS analysis was 96.4%. Analysis condition A: Retention time=1.6 min; ESI-MS(+) m/z [M+H]⁺: 1892.2.

### Preparation of Compound 1752

Compound 1752 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-D-Thr(tBu)-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 20.9 mg, and its estimated purity by LCMS analysis was 99.4%. Analysis condition A: Retention time=1.5 min; ESI-MS(+) m/z [M+2H]²⁺: 932.2.

### Preparation of Compound 1753

Compound 1753 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Nle-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 19 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 12.2 mg, and its estimated purity by LCMS analysis was 99.4%. Analysis condition B: Retention time=1.98 min; ESI-MS(+) m/z [M+H]⁺: 1890.1.

### Preparation of Compound 1754

Compound 1754 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 20 minutes, then a 5-minute hold at 100% B; Flow: 20 mL/min. Fractions containing the desired product were combined and dried via centrifugal evaporation. The yield of the product was 28.2 mg, and its estimated purity by LCMS analysis was 97.4%. Analysis condition A: Retention time=1.33 min; ESI-MS(+) m/z [M+H]⁺: 1843.2.

### Preparation of Compound 1755

Compound 1755 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Nva-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 19 minutes, then a 5-minute hold at 100% B; Flow: 20 mL/min. Fractions containing the desired product were combined and dried via centrifugal evaporation. The yield of the product was 25.9 mg, and its estimated purity by LCMS analysis was 100%. Analysis condition A: Retention time=1.64 min; ESI-MS(+) m/z [M+2H]²⁺: 938.8.

### Preparation of Compound 1756

Compound 1756 was prepared, using the crude product of Compound 1764. After ether trituration, the resulting solid was treated with a solution of TFA/Water (9:1; v:v) for 2.5 hours, after which time LCMS showed complete hydrolysis. Addition of ether (40 mL) resulted in formation of an off-white precipitate, which was collected by centrifugation, washed with ether (3×15 mL), redissolved in DMF, filtered and submitted to purification.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 3.3 mg, and its estimated purity by LCMS analysis was 92.7%. Analysis condition B: Retention time=1.5 min; ESI-MS(+) m/z [M+2H]²⁺: 965.

### Preparation of Compound 1757

Compound 1757 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 6.8 mg, and its estimated purity by LCMS analysis was 92.8%. Analysis condition A: Retention time=1.6 min; ESI-MS(+) m/z [M+H]⁺: 1820.

### Preparation of Compound 1758

Compound 1758 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Single-Coupling Manual Addition Procedure B” was followed with Fmoc-Phe(4-COOtBu)-OH and Fmoc-Ala(3-Pyr)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 12-52% B over 20 minutes, then a 3-minute hold at 100% B; Flow: 20 mL/min. Fractions containing the desired product were combined and dried via centrifugal evaporation. The yield of the product was 14.3 mg, and its estimated purity by LCMS analysis was 98.5%. Analysis condition B: Retention time=1.37 min; ESI-MS(+) m/z [M+H]⁺: 1858.

### Preparation of Compound 1759

Compound 1759A. Fmoc-Phe-Tyr(tBu)-Asp(tBu)-Trp(Boc)-Leu-Phe(4-Br)-Val-NMe-Ala-D-Ala-Asn(Trt)-Leu-Val-Ser(tBu)-Cys(Trt)-Ala-Rink amide resin was prepared, on a 100 μmol scale, following the general synthetic sequence described for the preparation of Compound 1000 composed of the following general procedures: “Symphony Resin-swelling procedure”, “Symphony Single-coupling procedure”.

Compound 1759. The peptidic resin of Compound 1759A (ca. 25 umol) and 3-fluorophenylboronic acid (10 equiv) were coupled using the general synthetic procedure “Suzuki On-resin Coupling Procedure” and then using the following general procedures: “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 1.5 mg, and its estimated purity by LCMS analysis was 96.7%. Analysis condition B: Retention time=1.72 min; ESI-MS(+) m/z [M+H]⁺: 1865.6.

### Preparation of Compound 1760

Compound 1760 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 20 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 10-60% B over 20 minutes, then a 2-minute hold at 100% B; Flow: 40 mL/min. Fractions containing the desired product were combined and dried via centrifugal evaporation. The yield of the product was 15.1 mg, and its estimated purity by LCMS analysis was 96.3%. Analysis condition A: Retention time=1.43 min; ESI-MS(+) m/z [M+H]⁺: 1866.1.

### Preparation of Compound 1761

Compound 1761 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Single-Coupling Manual Addition Procedure B” was followed with Fmoc-Phe(4-COOtBu)-OH and Fmoc-Ala(3-Pyr)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 20 minutes, then a 5-minute hold at 100% B; Flow: 20 mL/min. Fractions containing the desired product were combined and dried via centrifugal evaporation. The yield of the product was 23.4 mg, and its estimated purity by LCMS analysis was 96.9%. Analysis condition B: Retention time=1.28 min; ESI-MS(+) m/z [M+3H]³⁺: 641.2.

### Preparation of Compound 1762

Compound 1762 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 4-minute hold at 100% B; Flow: 20 mL/min. Fractions containing the desired product were combined and dried via centrifugal evaporation. The yield of the product was 9.3 mg, and its estimated purity by LCMS analysis was 97.1%. Analysis condition A: Retention time=1.47 min; ESI-MS(+) m/z [M+H]⁺: 1815.2.

### Preparation of Compound 1763

Compound 1763 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 18-58% B over 20 minutes, then a 5-minute hold at 100% B; Flow: 20 mL/min. Fractions containing the desired product were combined and dried via centrifugal evaporation. The yield of the product was 31.1 mg, and its estimated purity by LCMS analysis was 99.4%. Analysis condition B: Retention time=1.85 min; ESI-MS(+) m/z [M+H]⁺: 1862.2.

### Preparation of Compound 1764

Compound 1764 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Tyr(PO(NMe₂)₂)—OH; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 5-55% B over 20 minutes, then a 2-minute hold at 100% B; Flow: 45 mL/min. Fractions containing the desired product were combined and dried via centrifugal evaporation. The yield of the product was 8.8 mg, and its estimated purity by LCMS analysis was 97.7%. Analysis condition A: Retention time=1.87 min; ESI-MS(+) m/z [M+H]⁺: 1982.6.

### Preparation of Compound 1765

Compound 1765 was prepared, using Sieber or Rink on a 25 μmol scale, following the general synthetic sequence described for the preparation of Compounds 1000-10005 and using the general procedures described previously. The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 4-minute hold at 100% B; Flow: 20 mL/min. Fractions containing the desired product were combined and dried via centrifugal evaporation. The yield of the product was 11 mg, and its estimated purity by LCMS analysis was 100%. Analysis condition A: Retention time=1.63 min; ESI-MS(+) m/z [M+2H]²⁺: 925.2.

### Preparation of Compound 1766

Compound 1766 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 18-58% B over 25 minutes, then a 5-minute hold at 100% B; Flow: 20 mL/min. Fractions containing the desired product were combined and dried via centrifugal evaporation. The yield of the product was 38.6 mg, and its estimated purity by LCMS analysis was 92.7%. Analysis condition B: Retention time=1.76 min; ESI-MS(+) m/z [M+H]⁺: 1892.2.

### Preparation of Compound 1767

Compound 1767 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: X Bridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 20.1 mg, and its estimated purity by LCMS analysis was 98%. Analysis condition A: Retention time=1.41 min; ESI-MS(+) m/z [M+2H]²⁺: 947.1.

### Preparation of Compound 1768

Compound 1768 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 19.3 mg, and its estimated purity by LCMS analysis was 99.1%. Analysis condition A: Retention time=min; ESI-MS(+) m/z [M+H]⁺: 1877.84, 1877.84.

### Preparation of Compound 1769

Compound 1769 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 25 minutes, then a 5-minute hold at 100% B; Flow: 20 mL/min. Fractions containing the desired product were combined and dried via centrifugal evaporation. The yield of the product was 7.3 mg, and its estimated purity by LCMS analysis was 94.8%. Analysis condition A: Retention time=1.33 min; ESI-MS(+) m/z [M+H]⁺: 1873.1.

### Preparation of Compound 1770

Compound 1770 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 20-60% B over 27 minutes, then a 3-minute hold at 100% B; Flow: 20 mL/min. Fractions containing the desired product were combined and dried via centrifugal evaporation. The yield of the product was 34 mg, and its estimated purity by LCMS analysis was 96.2%. Analysis condition B: Retention time=1.68 min; ESI-MS(+) m/z [M+2H]²⁺: 960.1.

### Preparation of Compound 1771

Compound 1771 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 9.4 mg, and its estimated purity by LCMS analysis was 90.2%. Analysis condition A: Retention time=1.76 min; ESI-MS(+) m/z [M+H]⁺: 1876.

### Preparation of Compound 1772

Compound 1772 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Phe(3-OCH₂CH═CH₂)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 12.7 mg, and its estimated purity by LCMS analysis was 97.1%. Analysis condition B: Retention time=2.19 min; ESI-MS(+) m/z [M+H]⁺: 1889.2.

### Preparation of Compound 1773

Compound 1773 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Single-Coupling Manual Addition Procedure B” was followed with Fmoc-Phe(4-COOtBu)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 5-50% B over 20 minutes, then a 5-minute hold at 100% B; Flow: 20 mL/min. Fractions containing the desired product were combined and dried via centrifugal evaporation. The yield of the product was 36.7 mg, and its estimated purity by LCMS analysis was 84%. Analysis condition B: Retention time=1.65 min; ESI-MS(+) m/z [M+2H]²⁺: 932.2.

### Preparation of Compound 1774

Compound 1774 was prepared, using Sieber or Rink on a 25 μmol scale, following the general synthetic sequence described for the preparation of Compound 1759 composed of the following general procedures: “Symphony Resin-swelling procedure”, “Symphony Single-coupling procedure”, “On-resin Suzuki coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 4-minute hold at 100% B; Flow: 20 mL/min. Fractions containing the desired product were combined and dried via centrifugal evaporation. The yield of the product was 4.9 mg, and its estimated purity by LCMS analysis was 87.6%. Analysis condition A: Retention time=1.67 min; ESI-MS(+) m/z [M+2H]²⁺: 942.1.

### Preparation of Compound 1775

Compound 1775 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 30-70% B over 20 minutes, then a 5-minute hold at 100% B; Flow: 20 mL/min. Fractions containing the desired product were combined and dried via centrifugal evaporation. The yield of the product was 20.8 mg, and its estimated purity by LCMS analysis was 100%. Analysis condition B: Retention time=2.07 min; ESI-MS(+) m/z [M+H]⁺: 1947.2.

### Preparation of Compound 1776

Compound 1776 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Asn(Trt)-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 20 minutes, then a 5-minute hold at 100% B; Flow: 20 mL/min. Fractions containing the desired product were combined and dried via centrifugal evaporation. The yield of the product was 23.9 mg, and its estimated purity by LCMS analysis was 99.1%. Analysis condition B: Retention time=1.64 min; ESI-MS(+) m/z [M+2H]²⁺: 939.1.

### Preparation of Compound 1777

Compound 1777 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Phe(3-CF₃)—OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 36.9 mg, and its estimated purity by LCMS analysis was 100%. Analysis condition A: Retention time=1.87 min; ESI-MS(+) m/z [M+H]⁺: 1900.1.

### Preparation of Compound 1778

Compound 1778 was prepared, using Sieber or Rink on a 50 μmol scale, following the general synthetic sequence described for the preparation Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-60% B over 25 minutes, then a 5-minute hold at 100% B; Flow: 20 mL/min. Fractions containing the desired product were combined and dried via centrifugal evaporation. The yield of the product was 25.6 mg, and its estimated purity by LCMS analysis was 100%. Analysis condition A: Retention time=1.63 min; ESI-MS(+) m/z [M+H]⁺: 1862.

### Preparation of Compound 1779

Compound 1779 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 3-minute hold at 100% B; Flow: 20 mL/min. Fractions containing the desired product were combined and dried via centrifugal evaporation. The yield of the product was 20.1 mg, and its estimated purity by LCMS analysis was 100%. Analysis condition A: Retention time=1.75 min; ESI-MS(+) m/z [M+H]⁺: 1876.3.

### Preparation of Compound 1780

Compound 1780 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-55% B over 20 minutes, then a 3-minute hold at 100% B; Flow: 20 mL/min. Fractions containing the desired product were combined and dried via centrifugal evaporation. The yield of the product was 24.3 mg, and its estimated purity by LCMS analysis was 96.2%. Analysis condition B: Retention time=1.88 min; ESI-MS(+) m/z [M+H]⁺: 1989.2.

### Preparation of Compound 1781

Compound 1781 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 3-minute hold at 100% B; Flow: 20 mL/min. Fractions containing the desired product were combined and dried via centrifugal evaporation. The yield of the product was 32.8 mg, and its estimated purity by LCMS analysis was 96.2%. Analysis condition A: Retention time=1.73 min; ESI-MS(+) m/z [M+H]⁺: 1906.

### Preparation of Compound 1782

Compound 1782 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-60% B over 20 minutes, then a 3-minute hold at 100% B; Flow: 20 mL/min. Fractions containing the desired product were combined and dried via centrifugal evaporation. The yield of the product was 20.7 mg, and its estimated purity by LCMS analysis was 96.7%. Analysis condition A: Retention time=2.01 min; ESI-MS(+) m/z [M+H]⁺: 1948.

### Preparation of Compound 1783

Compound 1783 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 6.4 mg, and its estimated purity by LCMS analysis was 90%. Analysis condition B: Retention time=2.05 min; ESI-MS(+) m/z [M+H]⁺: 1923.9.

### Preparation of Compound 1784

Compound 1784 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-D-Gln-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 22.7 mg, and its estimated purity by LCMS analysis was 100%. Analysis condition A: Retention time=1.58 min; ESI-MS(+) m/z [M+H]⁺: 1890.7.

### Preparation of Compound 1785

Compound 1785 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 8.4 mg, and its estimated purity by LCMS analysis was 95.7%. Analysis condition B: Retention time=1.74 min; ESI-MS(+) m/z [M+H]⁺: 1834.9.

### Preparation of Compound 1786

Compound 1786 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”,

“Symphony X Single-Coupling Manual Addition Procedure B” was followed with Fmoc-Phe(4-COOtBu)-OH and Fmoc-Ala(3-Pyr)-OH; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 12-52% B over 20 minutes, then a 5-minute hold at 100% B; Flow: 20 mL/min. Fractions containing the desired product were combined and dried via centrifugal evaporation. The yield of the product was 21.3 mg, and its estimated purity by LCMS analysis was 88.8%. Analysis condition B: Retention time=1.49 min; ESI-MS(+) m/z [M+H]⁺: 1863.8.

### Preparation of Compound 1787

Compound 1787 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 27.1 mg, and its estimated purity by LCMS analysis was 91.7%. Analysis condition B: Retention time=1.61 min; ESI-MS(+) m/z [M+2H]²⁺: 932.1.

### Preparation of Compound 1788

Compound 1788 was prepared, using Sieber or Rink on a 25 μmol scale, following the general synthetic sequence described for the preparation of Compound 1759 composed of the following general procedures: “Symphony Resin-swelling procedure”, “Symphony Single-coupling procedure”, “On-resin Suzuki coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 4-minute hold at 100% B; Flow: 20 mL/min. Fractions containing the desired product were combined and dried via centrifugal evaporation. The material was further purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 1.7 mg, and its estimated purity by LCMS analysis was 100%. Analysis condition A: Retention time=1.67 min; ESI-MS(+) m/z [M+H]⁺: 1862.

### Preparation of Compound 1789

Compound 1789 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 37.8 mg, and its estimated purity by LCMS analysis was 98.4%. Analysis condition B: Retention time=1.75 min; ESI-MS(+) m/z [M+H]⁺: 1920.

### Preparation of Compound 1790

Compound 1790 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 28.6 mg, and its estimated purity by LCMS analysis was 98.2%. Analysis condition A: Retention time=1.7 min; ESI-MS(+) m/z [M+H]⁺: 1891.

### Preparation of Compound 1791

Compound 1791 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-70% B over 20 minutes, then a 5-minute hold at 100% B; Flow: 20 mL/min. Fractions containing the desired product were combined and dried via centrifugal evaporation. The yield of the product was 27.4 mg, and its estimated purity by LCMS analysis was 98.6%. Analysis condition B: Retention time=1.77 min; ESI-MS(+) m/z [M+H]⁺: 1848.1.

### Preparation of Compound 1792

Compound 1792 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 16.6 mg, and its estimated purity by LCMS analysis was 99.4%. Analysis condition A: Retention time=1.35 min; ESI-MS(+) m/z [M+2H]²⁺: 947.3.

### Preparation of Compound 1793

Compound 1793 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Lys-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 23.6 mg, and its estimated purity by LCMS analysis was 100%. Analysis condition A: Retention time=1.61 min; ESI-MS(+) m/z [M+H]⁺: 1905.1.

### Preparation of Compound 1794

Compound 1794 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 19 minutes, then a 5-minute hold at 100% B; Flow: 20 mL/min. Fractions containing the desired product were combined and dried via centrifugal evaporation. The yield of the product was 21.6 mg, and its estimated purity by LCMS analysis was 96.8%. Analysis condition A: Retention time=2 min; ESI-MS(+) m/z [M+H]⁺: 1845.9.

### Preparation of Compound 1795

Compound 1795 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Dab(Boc)-OH, “Symphony Chloroacetic Anhydride coupling procedure”, “Global Deprotection Method A” and “Cyclization Method”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 5-55% B over 20 minutes, then a 2-minute hold at 100% B; Flow: 40 mL/min. Fractions containing the desired product were combined and dried via centrifugal evaporation. The yield of the product was 21.3 mg, and its estimated purity by LCMS analysis was 100%. Analysis condition A: Retention time=1.74 min; ESI-MS(+) m/z [M+2H]²⁺: 932.2.

### Preparation of Compound 1796

Compound 1796 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Trp(2-Aza,7-Me)-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 20 minutes, then a 5-minute hold at 100% B; Flow: 20 mL/min. Fractions containing the desired product were combined and dried via centrifugal evaporation. The yield of the product was 24.6 mg, and its estimated purity by LCMS analysis was 99.1%. Analysis condition A: Retention time=1.65 min; ESI-MS(+) m/z [M+H]⁺: 1863.2.

### Preparation of Compound 1797

Compound 1797 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-D-His-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 42.6 mg, and its estimated purity by LCMS analysis was 96.4%. Analysis condition B: Retention time=1.62 min; ESI-MS(+) m/z [M+2H]²⁺: 951.2.

### Preparation of Compound 1798

Compound 1798 was prepared, using Sieber or Rink on a 25 μmol scale, following the general synthetic sequence described for the preparation of Compound 1759 composed of the following general procedures: “Symphony Resin-swelling procedure”, “Symphony Single-coupling procedure”, “On-resin Suzuki coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-60% B over 20 minutes, then a 4-minute hold at 100% B; Flow: 20 mL/min. Fractions containing the desired product were combined and dried via centrifugal evaporation. The yield of the product was 1.5 mg, and its estimated purity by LCMS analysis was 92.4%. Analysis condition B: Retention time=1.69 min; ESI-MS(+) m/z [M+H]⁺: 1878.8.

### Preparation of Compound 1799

Compound 1799 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-55% B over 25 minutes, then a 5-minute hold at 100% B; Flow: 20 mL/min. Fractions containing the desired product were combined and dried via centrifugal evaporation. The yield of the product was 31.2 mg, and its estimated purity by LCMS analysis was 93.7%. Analysis condition B: Retention time=1.59 min; ESI-MS(+) m/z [M+H]⁺: 1897.1.

### Preparation of Compound 1800

Compound 1800 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 7.7 mg, and its estimated purity by LCMS analysis was 95.8%. Analysis condition B: Retention time=1.91 min; ESI-MS(+) m/z [M+2H]²⁺: 942.

### Preparation of Compound 1801

Compound 1801 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Dap(Boc)-OH, “Symphony Chloroacetic Anhydride coupling procedure”, “Global Deprotection Method A” and “Cyclization Method”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 5-55% B over 20 minutes, then a 2-minute hold at 100% B; Flow: 40 mL/min. Fractions containing the desired product were combined and dried via centrifugal evaporation. The yield of the product was 20.5 mg, and its estimated purity by LCMS analysis was 99.1%. Analysis condition A: Retention time=1.71 min; ESI-MS(+) m/z [M+2H]²⁺: 925.4.

### Preparation of Compound 1802

Compound 1802 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Dap(Boc)-OH, “Symphony Chloroacetic Anhydride coupling procedure”, “Global Deprotection Method A”, and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 19 minutes, then a 5-minute hold at 100% B; Flow: 20 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 13 mg, and its estimated purity by LCMS analysis was 99.2%. Analysis condition A: Retention time=1.62 min; ESI-MS(+) m/z [M+2H]²⁺: 946.

### Preparation of Compound 1803

Compound 1803 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Nva-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 29.7 mg, and its estimated purity by LCMS analysis was 95.8%. Analysis condition A: Retention time=1.79 min; ESI-MS(+) m/z [M+2H]²⁺: 931.8.

### Preparation of Compound 1804

Compound 1804 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure”; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 15-65% B over 20 minutes, then a 2-minute hold at 100% B; Flow: 40 mL/min. Fractions containing the desired product were combined and dried via centrifugal evaporation. The yield of the product was 9.7 mg, and its estimated purity by LCMS analysis was 98.9%. Analysis condition B: Retention time=1.58 min; ESI-MS(+) m/z [M+2H]²⁺: 967.5.

Compound 1805 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 11.5 mg, and its estimated purity by LCMS analysis was 100%. Analysis condition A: Retention time=1.88 min; ESI-MS(+) m/z [M+H]⁺: 1882.1.

### Preparation of Compound 1806

Compound 1806 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 30-70% B over 20 minutes, then a 5-minute hold at 100% B; Flow: 20 mL/min. Fractions containing the desired product were combined and dried via centrifugal evaporation. The yield of the product was 12 mg, and its estimated purity by LCMS analysis was 98.2%. Analysis condition A: Retention time=1.68 min; ESI-MS(+) m/z [M+2H]²⁺: 929.1.

### Preparation of Compound 1807

Compound 1807 was prepared, using Sieber or Rink on a 50 μmol scale, following the general synthetic sequence described for the preparation of Compound 1002, composed of the following general procedures: “Symphony Resin-swelling procedure”, “Symphony Single-coupling procedure”, “Symphony Single-Coupling Pre-activation Procedure”; “Symphony X Resin-swelling procedure”, “Symphony X Single-coupling procedure”, Single-Coupling Manual Addition Procedure B”, “Symphony X Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 40-80% B over 20 minutes, then a 4-minute hold at 100% B; Flow: 20 mL/min. Fractions containing the desired product were combined and dried via centrifugal evaporation. The yield of the product was 41.2 mg, and its estimated purity by LCMS analysis was 95.8%. Analysis condition A: Retention time=1.85 min; ESI-MS(+) m/z [M+H]⁺: 1866.1.

### Preparation of Compound 1808

Compound 1808 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 17-57% B over 25 minutes, then a 5-minute hold at 100% B; Flow: 20 mL/min. Fractions containing the desired product were combined and dried via centrifugal evaporation. The yield of the product was 57.2 mg, and its estimated purity by LCMS analysis was 95.9%. Analysis condition B: Retention time=1.62 min; ESI-MS(+) m/z [M+H]⁺: 1866.9.

### Preparation of Compound 1809

Compound 1809 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 28.5 mg, and its estimated purity by LCMS analysis was 98.6%. Analysis condition A: Retention time=1.69 min; ESI-MS(+) m/z [M+2H]²⁺: 960.1.

### Preparation of Compound 1810

Compound 1810 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 12 mg, and its estimated purity by LCMS analysis was 100%. Analysis condition A: Retention time=2.1 min; ESI-MS(+) m/z [M+H]⁺: 1900.2.

### Preparation of Compound 1811

Compound 1811 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 10-60% B over 20 minutes, then a 2-minute hold at 100% B; Flow: 40 mL/min. Fractions containing the desired product were combined and dried via centrifugal evaporation. The yield of the product was 43.1 mg, and its estimated purity by LCMS analysis was 99.1%. Analysis condition A: Retention time=1.58 min; ESI-MS(+) m/z [M+2H]²⁺: 946.5.

### Preparation of Compound 1812

Compound 1812 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-Cit-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 7.7 mg, and its estimated purity by LCMS analysis was 97%. Analysis condition B: Retention time=1.71 min; ESI-MS(+) m/z [M+2H]²⁺: 961.3.

### Preparation of Compound 1813

Compound 1813 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Tyr(Ph)-OH; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 20 mg, and its estimated purity by LCMS analysis was 92.7%. Analysis condition B: Retention time=1.99 min; ESI-MS(+) m/z [M+2H]²⁺: 963.

### Preparation of Compound 1814

Compound 1814 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-homo-Ser(tBu)-OH, “Symphony Chloroacetic Anhydride coupling procedure”, “Global Deprotection Method A” and “Cyclization Method”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 5-55% B over 20 minutes, then a 2-minute hold at 100% B; Flow: 40 mL/min. Fractions containing the desired product were combined and dried via centrifugal evaporation. The yield of the product was 31.6 mg, and its estimated purity by LCMS analysis was 100%. Analysis condition A: Retention time=min; ESI-MS(+) m/z [M+2H]²⁺: 933.5.

### Preparation of Compound 1815

Compound 1815 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 20 minutes, then a 5-minute hold at 100% B; Flow: 20 mL/min. Fractions containing the desired product were combined and dried via centrifugal evaporation. The yield of the product was 36.9 mg, and its estimated purity by LCMS analysis was 100%. Analysis condition A: Retention time=1.53 min; ESI-MS(+) m/z [M+2H]²⁺: 922.0.

### Preparation of Compound 1816

Compound 1816 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony double-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 25 minutes, then a 5-minute hold at 100% B; Flow: 20 mL/min. Fractions containing the desired product were combined and dried via centrifugal evaporation. The yield of the product was 15 mg, and its estimated purity by LCMS analysis was 99.2%. Analysis condition B: Retention time=1.76 min; ESI-MS(+) m/z [M+H]⁺: 1919.1.

### Preparation of Compound 1817

Compound 1817 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-60% B over 20 minutes, then a 5-minute hold at 100% B; Flow: 20 mL/min. Fractions containing the desired product were combined and dried via centrifugal evaporation. The yield of the product was 18.6 mg, and its estimated purity by LCMS analysis was 100%. Analysis condition A: Retention time=1.55 min; ESI-MS(+) m/z [M+2H]²⁺: 947.3.

### Preparation of Compound 1818

Compound 1818 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×150 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-65% B over 20 minutes, then a 2-minute hold at 100% B; Flow: 40 mL/min. Fractions containing the desired product were combined and dried via centrifugal evaporation. The yield of the product was 28.5 mg, and its estimated purity by LCMS analysis was 98.7%. Analysis condition A: Retention time=1.76 min; ESI-MS(+) m/z [M+H]⁺: 1848.9.

### Preparation of Compound 1819

Compound 1819 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Ser(tBu)-OH, “Symphony Chloroacetic Anhydride coupling procedure”, “Global Deprotection Method A” and “Cyclization Method”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 5-55% B over 20 minutes, then a 2-minute hold at 100% B; Flow: 40 mL/min. Fractions containing the desired product were combined and dried via centrifugal evaporation. The yield of the product was 19.7 mg, and its estimated purity by LCMS analysis was 90.9%. Analysis condition A: Retention time=1.49 min; ESI-MS(+) m/z [M+2H]²⁺: 925.9.

### Preparation of Compound 1820

Compound 1820 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony X Resin-swelling procedure”, “Symphony X Single-coupling procedure”, “Symphony X Single-Coupling Single-shot Procedure”; “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 5-55% B over 20 minutes, then a 2-minute hold at 100% B; Flow: 45 mL/min. Fractions containing the desired product were combined and dried via centrifugal evaporation. The yield of the product was 7.1 mg, and its estimated purity by LCMS analysis was 95.8%. Analysis condition B: Retention time=1.61 min; ESI-MS(+) m/z [M+2H]²⁺: 954.2.

### Preparation of Compound 1821

Compound 1821 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure” was followed with Fmoc-Ser(P03H₂)—OH; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 5-55% B over 20 minutes, then a 2-minute hold at 100% B; Flow: 45 mL/min. Fractions containing the desired product were combined and dried via centrifugal evaporation. The yield of the product was 12.5 mg, and its estimated purity by LCMS analysis was 99.3%. Analysis condition A: Retention time=1.71 min; ESI-MS(+) m/z [M+H]⁺: 1900.2.

### Preparation of Compound 1822

Compound 1822 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 20 minutes, then a 5-minute hold at 100% B; Flow: 20 mL/min. Fractions containing the desired product were combined and dried via centrifugal evaporation. The yield of the product was 34.1 mg, and its estimated purity by LCMS analysis was 100%. Analysis condition A: Retention time=1.49 min; ESI-MS(+) m/z [M+H]⁺: 1843.8.

### Preparation of Compound 1823

Compound 1823 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-70% B over 20 minutes, then a 2-minute hold at 100% B; Flow: 45 mL/min. Fractions containing the desired product were combined and dried via centrifugal evaporation. The yield of the product was 5.8 mg, and its estimated purity by LCMS analysis was 91%. Analysis condition A: Retention time=1.85 min; ESI-MS(+) m/z [M+H]⁺: 1909.8.

### Preparation of Compound 1824

Compound 1824 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 40-80% B over 20 minutes, then a 5-minute hold at 100% B; Flow: 20 mL/min. Fractions containing the desired product were combined and dried via centrifugal evaporation. The yield of the product was 38.8 mg, and its estimated purity by LCMS analysis was 97.3%. Analysis condition B: Retention time=2.02 min; ESI-MS(+) m/z [M+2H]²⁺: 939.2.

### Preparation of Compound 1825

Compound 1825 was prepared, using Sieber or Rink on a 25 μmol scale, following the general synthetic sequence described for the preparation of Compound 1759 composed of the following general procedures: “Symphony Resin-swelling procedure”, “Symphony Single-coupling procedure”, “On-resin Suzuki coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 3 mg, and its estimated purity by LCMS analysis was 100%. Analysis condition A: Retention time=1.44 min; ESI-MS(+) m/z [M+2H]²⁺: 934.8.

### Preparation of Compound 1826

Compound 1827 was prepared, using Sieber or Rink on a 50 μmol scale, following the general synthetic sequence described for the preparation of Compound 1002, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method B”, “Cyclization Method A”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-65% B over 20 minutes, then a 2-minute hold at 100% B; Flow: 45 mL/min. Fractions containing the desired product were combined and dried via centrifugal evaporation. The yield of the product was 22.5 mg, and its estimated purity by LCMS analysis was 99.2%. Analysis condition A: Retention time=1.67 min; ESI-MS(+) m/z [M+2H]²⁺: 946.1.

### Preparation of Compound 1827

Compound 1827 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Tle-OH, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 30-70% B over 20 minutes, then a 4-minute hold at 100% B; Flow: 20 mL/min. Fractions containing the desired product were combined and dried via centrifugal evaporation. The yield of the product was 8 mg, and its estimated purity by LCMS analysis was 87.4%. Analysis condition B: Retention time=1.99 min; ESI-MS(+) m/z [M+2H]²⁺: 946.1.

### Preparation of Compound 1828

Compound 1828 was prepared, using Sieber or Rink on a 50 μmol scale, following the general synthetic sequence described for the preparation of Compound 1001, composed of the following general procedures: “Symphony X Resin-swelling procedure”, “Symphony X Single-coupling procedure”, “Symphony X Chloroacetic Anhydride coupling procedure”, “Symphony X Final rinse and dry procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 4 minutes, then a 5-minute hold at 100% B; Flow: 20 mL/min. Fractions containing the desired product were combined and dried via centrifugal evaporation. The yield of the product was 27.9 mg, and its estimated purity by LCMS analysis was 98.1%. Analysis condition B: Retention time=1.75 min; ESI-MS(+) m/z [M+H]⁺: 1842.9.

### Preparation of Compound 1829

Compound 1829 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, Single-Coupling Pre-Activation Procedure” for Fmoc-D-Asp(Boc)-OH, “Symphony Chloroacetic Anhydride coupling procedure”, “Global Deprotection Method A” and “Cyclization Method”.

The crude material was purified via preparative LC/MS with the following conditions: Column: XBridge C18, 30×150 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 5-55% B over 20 minutes, then a 2-minute hold at 100% B; Flow: 40 mL/min. Fractions containing the desired product were combined and dried via centrifugal evaporation. The yield of the product was 17.7 mg, and its estimated purity by LCMS analysis was 100%. Analysis condition A: Retention time=1.48 min; ESI-MS(+) m/z [M+H]⁺: 1877.7.

### Preparation of Compound 1830

Compound 1830 was prepared, using Sieber or Rink on a 25 μmol scale, following the general synthetic sequence described for the preparation of Compound 1759 composed of the following general procedures: “Symphony Resin-swelling procedure”, “Symphony Single-coupling procedure”, “On-resin Suzuki coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 20 minutes, then a 4-minute hold at 100% B; Flow: 20 mL/min. Fractions containing the desired product were combined and dried via centrifugal evaporation. The material was further purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 5-55% B over 20 minutes, then a 2-minute hold at 100% B; Flow: 45 mL/min. Fractions containing the desired product were combined and dried via centrifugal evaporation. The yield of the product was 2 mg, and its estimated purity by LCMS analysis was 97.5%. Analysis condition B: Retention time=1.65 min; ESI-MS(+) m/z [M+H]⁺: 1890.7.

### Preparation of Compound 1831

Compound 1831 was prepared, using Sieber or Rink on a 50 μmol scale, following the general synthetic sequence described for the preparation of Compound 1002, composed of the following general procedures: “Prelude Resin-swelling procedure”, “Prelude Single-coupling procedure”, “Symphony Resin-swelling procedure”, “Symphony Single-coupling procedure”, “Symphony Single-Coupling Pre-activation Procedure”; “Symphony Chloroacetic Anhydride coupling procedure”, “Global Deprotection Method A”, “Cyclization Method A”.

The crude material was purified via preparative LC/MS with the following conditions: Column: XBridge C18, 19×200 mm, 5-μm particles; Mobile Phase A: 5:95 acetonitrile:water with 0.1% trifluoroacetic acid; Mobile Phase B: 95:5 acetonitrile:water with 0.1% trifluoroacetic acid; Gradient: 40-80% B over 20 minutes, then a 5-minute hold at 100% B; Flow: 20 mL/min. Fractions containing the desired product were combined and dried via centrifugal evaporation. The yield of the product was 11.8 mg, and its estimated purity by LCMS analysis was 98.2%. Analysis condition 2: Retention time=2.17 min; ESI-MS(+) m/z [M+H]⁺: 1900.9.

### Preparation of Compound 1832

Compound 1832 was prepared, using Sieber or Rink on a 50 μmol scale, following the general synthetic sequence described for the preparation of Compound 1000 composed of the following general procedures: “Symphony Resin-swelling procedure”, “Symphony Single-coupling procedure”, “Symphony Chloroacetic Anhydride coupling procedure”, “Global Deprotection Method A” and “Cyclization Method”.

The crude material was purified via preparative LC/MS with the following conditions: Column: XBridge C18, 30×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-60% B over 20 minutes, then a 2-minute hold at 100% B; Flow: 45 mL/min. Fractions containing the desired product were combined and dried via centrifugal evaporation. The yield of the product was 8.5 mg, and its estimated purity by LCMS analysis was 99.1%. Analysis condition A: Retention time=1.41 min; ESI-MS(+) m/z [M+2H]²⁺: 960.1.

### Preparation of Compound 1833

Compound 1833 was prepared on a 50 μmol scale. The yield of the product was 40.4 mg, and its estimated purity by LCMS analysis was 98.7%. Analysis condition B: Retention time=1.79 min; ESI-MS(+) m/z [M+H]⁺: 1859.9.

### Preparation of Compound 1834

Compound 1834 was prepared on a 30 μmol scale. The yield of the product was 15.3 mg, and its estimated purity by LCMS analysis was 91%. Analysis condition B: Retention time=1.86 min; ESI-MS(+) m/z [M+2H]²⁺: 1096.1.

### Preparation of Compound 1835

Compound 1835 was prepared on a 30 μmol scale. The yield of the product was 16.8 mg, and its estimated purity by LCMS analysis was 92.3%. Analysis condition B: Retention time=1.71 min; ESI-MS(+) m/z [M+3H]³⁺: 773.1.

### Preparation of Compound 1836

Compound 1836 was prepared on a 30 μmol scale. The yield of the product was 26.4 mg, and its estimated purity by LCMS analysis was 97.2%. Analysis condition A: Retention time=1.45 min; ESI-MS(+) m/z [M+2H]²⁺: 1135.1.

### Preparation of Compound 1837

Compound 1837 was prepared on a 30 μmol scale. The yield of the product was 23.9 mg, and its estimated purity by LCMS analysis was 95%. Analysis condition A: Retention time=1.53 min; ESI-MS(+) m/z [M+3H]³⁺: 747.1.

### Preparation of Compound 1838

Compound 1838 was prepared on a 30 μmol scale. The yield of the product was 11.2 mg, and its estimated purity by LCMS analysis was 95.4%. Analysis condition A: Retention time=1.67 min; ESI-MS(+) m/z [M+2H]²⁺: 1037.9.

### Preparation of Compound 1839

Compound 1839 was prepared on a 30 μmol scale. The yield of the product was 7.6 mg, and its estimated purity by LCMS analysis was 82.9%. Analysis condition A: Retention time=1.62 min; ESI-MS(+) m/z [M+2H]²⁺: 1067.2.

### Preparation of Compound 1840

Compound 1840 was prepared on a 30 μmol scale. The yield of the product was 17.6 mg, and its estimated purity by LCMS analysis was 94.5%. Analysis condition A: Retention time=2.13 min; ESI-MS(+) m/z [M+2H]²⁺: 1016.2.

### Preparation of Compound 1841

Compound 1841 was prepared on a 30 μmol scale. The yield of the product was 31.4 mg, and its estimated purity by LCMS analysis was 96.9%. Analysis condition A: Retention time=1.93 min; ESI-MS(+) m/z [M+2H]²⁺: 1077.2.

### Preparation of Compound 1842

Compound 1842 was prepared on a 30 μmol scale. The yield of the product was 26.3 mg, and its estimated purity by LCMS analysis was 95.9%. Analysis condition A: Retention time=1.84 min; ESI-MS(+) m/z [M+2H]²⁺: 1050.9.

### Preparation of Compound 1843

Compound 1843 was prepared on a 30 μmol scale. The yield of the product was 27.4 mg, and its estimated purity by LCMS analysis was 95.1%. Analysis condition A: Retention time=1.84 min; ESI-MS(+) m/z [M+2H]²⁺: 1075.1.

### Preparation of Compound 1844

Compound 1844 was prepared on a 30 μmol scale. The yield of the product was 27.9 mg, and its estimated purity by LCMS analysis was 100%. Analysis condition 3: Retention time=1.62 min; ESI-MS(+) m/z [M+2H]²⁺: 1076.2.

### Preparation of Compound 1845

Compound 1845 was prepared on a 30 μmol scale. The yield of the product was 10.1 mg, and its estimated purity by LCMS analysis was 91.5%. Analysis condition A: Retention time=1.51 min; ESI-MS(+) m/z [M+2H]²⁺: 1099.2.

### Preparation of Compound 1846

Compound 1846 was prepared on a 50 μmol scale. The yield of the product was 2.4 mg, and its estimated purity by LCMS analysis was 91%. Analysis condition B: Retention time=1.71 min; ESI-MS(+) m/z [M+2H]²⁺: 1051.1.

### Preparation of Compound 1847

Compound 1847 was prepared on a 50 μmol scale. The yield of the product was 33.8 mg, and its estimated purity by LCMS analysis was 91.6%. Analysis condition A: Retention time=1.47 min; ESI-MS(+) m/z [M+2H]²⁺: 1111.2.

### Preparation of Compound 1848

Compound 1848 was prepared on a 50 μmol scale. The yield of the product was 9.3 mg, and its estimated purity by LCMS analysis was 95.6%. Analysis condition A: Retention time=1.57 min; ESI-MS(+) m/z [M+2H]²⁺: 1103.2.

### Preparation of Compound 1849

Compound 1849 was prepared on a 30 μmol scale. The yield of the product was 34.5 mg, and its estimated purity by LCMS analysis was 97.1%. Analysis condition A: Retention time=1.76 min; ESI-MS(+) m/z [M+2H]²⁺: 1017.

### Preparation of Compound 1850

Compound 1850 was prepared on a 30 μmol scale. The yield of the product was 11.6 mg, and its estimated purity by LCMS analysis was 98.2%. Analysis condition A: Retention time=1.73 min; ESI-MS(+) m/z [M+H]⁺: 1984.2.

### Preparation of Compound 1851

Compound 1851 was prepared on a 50 μmol scale. The yield of the product was 13.6 mg, and its estimated purity by LCMS analysis was 92.4%. Analysis condition B: Retention time=1.92 min; ESI-MS(+) m/z [M+2H]²⁺: 1110.

### Preparation of Compound 1852

Compound 1852 was prepared on a 50 μmol scale. The yield of the product was 14.8 mg, and its estimated purity by LCMS analysis was 95.8%. Analysis condition B: Retention time=1.77 min; ESI-MS(+) m/z [M+2H]²⁺: 1121.1.

### Preparation of Compound 1853

Compound 1853 was prepared on a 50 μmol scale. The yield of the product was 53.4 mg, and its estimated purity by LCMS analysis was 98%. Analysis condition A: Retention time=1.44 min; ESI-MS(+) m/z [M+2H]²⁺: 1141.2.

### Preparation of Compound 1854

Compound 1854 was prepared on a 50 μmol scale. The yield of the product was 23.9 mg, and its estimated purity by LCMS analysis was 95%. Analysis condition B: Retention time=1.76 min; ESI-MS(+) m/z [M+2H]²⁺: 1134.

### Preparation of Compound 1855

Compound 1855 was prepared on a 25 μmol scale. The yield of the product was 32.3 mg, and its estimated purity by LCMS analysis was 91.8%. Analysis condition B: Retention time=1.86 min; ESI-MS(+) m/z [M+2H]²⁺: 1133.2.

### Preparation of Compound 1856

Compound 1856 was prepared on a 25 μmol scale. The yield of the product was 36.2 mg, and its estimated purity by LCMS analysis was 97.9%. Analysis condition B: Retention time=1.66 min; ESI-MS(+) m/z [M+2H]²⁺: 1126.2.

### Preparation of Compound 1857

Compound 1857 was prepared on a 25 μmol scale. The yield of the product was 32.9 mg, and its estimated purity by LCMS analysis was 97.2%. Analysis condition B: Retention time=1.74 min; ESI-MS(+) m/z [M+2H]²⁺: 1148.1.

### Preparation of Compound 1858

Compound 1858 was prepared on a 25 μmol scale. The yield of the product was 29.2 mg, and its estimated purity by LCMS analysis was 98.4%. Analysis condition A: Retention time=1.41 min; ESI-MS(+) m/z [M+2H]²⁺: 1126.2.

### Preparation of Compound 1859

Compound 1859 was prepared on a 25 μmol scale. The yield of the product was 10.2 mg, and its estimated purity by LCMS analysis was 97.8%. Analysis condition B: Retention time=1.8 min; ESI-MS(+) m/z [M+2H]²⁺: 1095.2.

### Preparation of Compound 1860

Compound 1860 was prepared on a 25 μmol scale. The yield of the product was 10.4 mg, and its estimated purity by LCMS analysis was 95.3%. Analysis condition A: Retention time=1.35 min; ESI-MS(+) m/z [M+2H]²⁺: 1105.0.

### Preparation of Compound 1861

Compound 1861 was prepared on a 25 μmol scale. The yield of the product was 15.8 mg, and its estimated purity by LCMS analysis was 98.6%. Analysis condition B: Retention time=1.82 min; ESI-MS(+) m/z [M+2H]²⁺: 1112.2.

### Preparation of Compound 1862

Compound 1862 was prepared on a 25 μmol scale. The yield of the product was 32.1 mg, and its estimated purity by LCMS analysis was 95%. Analysis condition A: Retention time=1.47 min; ESI-MS(+) m/z [M+2H]²⁺: 1073.2.

### Preparation of Compound 1863

Compound 1863 was prepared on a 25 μmol scale. The yield of the product was 27 mg, and its estimated purity by LCMS analysis was 96.2%. Analysis condition A: Retention time=1.47 min; ESI-MS(+) m/z [M+2H]²⁺: 1097.1.

### Preparation of Compound 1864

Compound 1864 was prepared on a 25 μmol scale. The yield of the product was 17.7 mg, and its estimated purity by LCMS analysis was 95.8%. Analysis condition A: Retention time=1.64 min; ESI-MS(+) m/z [M+2H]²⁺: 1123.1.

### Preparation of Compound 1865

Compound 1865 was prepared on a 25 μmol scale. The yield of the product was 29.8 mg, and its estimated purity by LCMS analysis was 91.1%. Analysis condition A: Retention time=1.67 min; ESI-MS(+) m/z [M+2H]²⁺: 1154.1.

### Preparation of Compound 1866

Compound 1866 was prepared on a 25 μmol scale. The yield of the product was 14.2 mg, and its estimated purity by LCMS analysis was 95.1%. Analysis condition B: Retention time=1.89 min; ESI-MS(+) m/z [M+3H]³⁺: 749.1.

### Preparation of Compound 1867

Compound 1867 was prepared on a 25 μmol scale. The yield of the product was 9.6 mg, and its estimated purity by LCMS analysis was 95.2%. Analysis condition A: Retention time=1.56 min; ESI-MS(+) m/z [M+2H]²⁺: 1154.0.

### Preparation of Compound 1868

Compound 1868 was prepared on a 25 μmol scale. The yield of the product was 7 mg, and its estimated purity by LCMS analysis was 95.6%. Analysis condition A: Retention time=2.19 min; ESI-MS(+) m/z [M+H]⁺: 1967.2.

### Preparation of Compound 1869

Compound 1869 was prepared on a 25 μmol scale. The yield of the product was 3.7 mg, and its estimated purity by LCMS analysis was 100%. Analysis condition A: Retention time=1.65 min; ESI-MS(+) m/z [M+2H]²⁺: 1005.2.

### Preparation of Compound 1870

Compound 1870 was prepared on a 50 μmol scale. The yield of the product was 52.3 mg, and its estimated purity by LCMS analysis was 98.7%. Analysis condition A: Retention time=1.56 min; ESI-MS(+) m/z [M+3H]³⁺: 720.1.

### Preparation of Compound 1871

Compound 1871 was prepared on a 50 μmol scale. The yield of the product was 55.6 mg, and its estimated purity by LCMS analysis was 94.7%. Analysis condition B: Retention time=1.59 min; ESI-MS(+) m/z [M+2H]²⁺: 1012.1.

### Preparation of Compound 1872

Compound 1872 was prepared on a 50 μmol scale. The yield of the product was 21.7 mg, and its estimated purity by LCMS analysis was 99.5%. Analysis condition B: Retention time=1.55 min; ESI-MS(+) m/z [M+3H]³⁺: 727.1.

### Preparation of Compound 1873

Compound 1873 was prepared on a 50 μmol scale. The yield of the product was 18 mg, and its estimated purity by LCMS analysis was 97.9%. Analysis condition B: Retention time=1.58 min; ESI-MS(+) m/z [M+2H]²⁺: 1023.2.

### Preparation of Compound 1874

Compound 1874 was prepared on a 50 μmol scale. The yield of the product was 9.9 mg, and its estimated purity by LCMS analysis was 97.4%. Analysis condition B: Retention time=1.35 min; ESI-MS(+) m/z [M+H]⁺: 1975.9.

### Preparation of Compound 1875

Compound 1875 was prepared on a 50 μmol scale. The yield of the product was 17.3 mg, and its estimated purity by LCMS analysis was 100%. Analysis condition A: Retention time=1.5 min; ESI-MS(+) m/z [M+2H]²⁺: 1009.2.

### Preparation of Compound 1876

Compound 1876 was prepared on a 50 μmol scale. The yield of the product was 6.8 mg, and its estimated purity by LCMS analysis was 98.6%. Analysis condition B: Retention time=1.6 min; ESI-MS(+) m/z [M+2H]²⁺: 992.

### Preparation of Compound 1877

Compound 1877 was prepared on a 50 μmol scale. The yield of the product was 15.3 mg, and its estimated purity by LCMS analysis was 91.8%. Analysis condition A: Retention time=1.75 min; ESI-MS(+) m/z [M+H]⁺: 1869.

### Preparation of Compound 1878

Compound 1878 was prepared on a 50 μmol scale. The yield of the product was 13.9 mg, and its estimated purity by LCMS analysis was 97.9%. Analysis condition B: Retention time=1.63 min; ESI-MS(+) m/z [M+2H]²⁺: 1052.1.

### Preparation of Compound 1879

Compound 1879 was prepared on a 50 μmol scale. The yield of the product was 24.1 mg, and its estimated purity by LCMS analysis was 96.1%. Analysis condition B: Retention time=1.83 min; ESI-MS(+) m/z [M+H]⁺: 1974.2.

### Preparation of Compound 1880

Compound 1880 was prepared on a 100 μmol scale. The yield of the product was 63.6 mg, and its estimated purity by LCMS analysis was 100%. Analysis condition A: Retention time=1.63 min; ESI-MS(+) m/z [M+H]⁺: 1904.1.

### Preparation of Compound 1881

Compound 1881 was prepared on a 100 μmol scale. The yield of the product was 90.3 mg, and its estimated purity by LCMS analysis was 97.3%. Analysis condition B: Retention time=2 min; ESI-MS(+) m/z [M+H]⁺: 1905.6.

### Preparation of Compound 1882

Compound 1882 was prepared on a 100 μmol scale. The yield of the product was 84.5 mg, and its estimated purity by LCMS analysis was 100%. Analysis condition A: Retention time=1.64 min; ESI-MS(+) m/z [M+H]⁺: 1893.2.

### Preparation of Compound 1883

Compound 1883 was prepared on a 100 μmol scale. The yield of the product was 92.5 mg, and its estimated purity by LCMS analysis was 93%. Analysis condition B: Retention time=1.88 min; ESI-MS(+) m/z [M+2H]²⁺: 946.4.

### Preparation of Compound 1884

Compound 1884 was prepared on a 100 μmol scale. The yield of the product was 59.7 mg, and its estimated purity by LCMS analysis was 100%. Analysis condition A: Retention time=1.69 min; ESI-MS(+) m/z [M+H]⁺: 1900.1.

### Preparation of Compound 1885

Compound 1885 was prepared on a 100 μmol scale. The yield of the product was 56.6 mg, and its estimated purity by LCMS analysis was 98.9%. Analysis condition B: Retention time=1.74 min; ESI-MS(+) m/z [M+2H]²⁺: 1029.1.

### Preparation of Compound 1886

Compound 1886 was prepared on a 100 μmol scale. The yield of the product was 90.2 mg, and its estimated purity by LCMS analysis was 96.2%. Analysis condition A: Retention time=1.5 min; ESI-MS(+) m/z [M+2H]²⁺: 1031.2.

### Preparation of Compound 1887

Compound 1887 was prepared on a 100 μmol scale. The yield of the product was 65.6 mg, and its estimated purity by LCMS analysis was 97%. Analysis condition A: Retention time=1.59 min; ESI-MS(+) m/z [M+2H]²⁺: 1023.2.

### Preparation of Compound 1888

Compound 1888 was prepared on a 100 μmol scale. The yield of the product was 79 mg, and its estimated purity by LCMS analysis was 95.5%. Analysis condition A: Retention time=1.49 min; ESI-MS(+) m/z [M+2H]²⁺: 1024.1.

### Preparation of Compound 1889

Compound 1889 was prepared on a 100 μmol scale. The yield of the product was 41.5 mg, and its estimated purity by LCMS analysis was 100%. Analysis condition B: Retention time=1.84 min; ESI-MS(+) m/z [M+3H]³⁺: 684.9.

### Preparation of Compound 1890

Compound 1890 was prepared on a 50 μmol scale. The yield of the product was 15.8 mg, and its estimated purity by LCMS analysis was 93.3%. Analysis condition A: Retention time=1.7 min; ESI-MS(+) m/z [M+2H]²⁺: 1051.1

### Preparation of Compound 1891

Compound 1891 was prepared on a 50 μmol scale. The yield of the product was 25.3 mg, and its estimated purity by LCMS analysis was 92.4%. Analysis condition A: Retention time=1.7 min; ESI-MS(+) m/z [M+2H]²⁺: 1045.

### Preparation of Compound 1892

Compound 1892 was prepared on a 50 μmol scale. The yield of the product was 18.4 mg, and its estimated purity by LCMS analysis was 95.9%. Analysis condition B: Retention time=1.7 min; ESI-MS(+) m/z [M+2H]²⁺: 1037.

### Preparation of Compound 1893

Compound 1893 was prepared on a 50 μmol scale. The yield of the product was 32.3 mg, and its estimated purity by LCMS analysis was 93.7%. Analysis condition A: Retention time=1.58 min; ESI-MS(+) m/z [M+2H]²⁺: 1051.

### Preparation of Compound 1894

Compound 1894 was prepared on a 50 μmol scale. The yield of the product was 8.8 mg, and its estimated purity by LCMS analysis was 98.3%. Analysis condition B: Retention time=1.79 min; ESI-MS(+) m/z [M+2H]²⁺: 1057.9.

### Preparation of Compound 1895

Compound 1895 was prepared on a 50 μmol scale. The yield of the product was 20.1 mg, and its estimated purity by LCMS analysis was 98%. Analysis condition A: Retention time=1.6 min; ESI-MS(+) m/z [M+2H]²⁺: 1050.9.

### Preparation of Compound 1896

Compound 1896 was prepared on a 50 μmol scale. The yield of the product was 28.4 mg, and its estimated purity by LCMS analysis was 92.9%. Analysis condition B: Retention time=1.77 min; ESI-MS(+) m/z [M+2H]²⁺: 1058.

### Preparation of Compound 1897

Compound 1897 was prepared on a 50 μmol scale. The yield of the product was 23.7 mg, and its estimated purity by LCMS analysis was 94.8%. Analysis condition B: Retention time=1.51 min; ESI-MS(+) m/z [M+3H]³⁺: 641.1.

### Preparation of Compound 1898

Compound 1898 was prepared on a 50 μmol scale. The yield of the product was 27.4 mg, and its estimated purity by LCMS analysis was 97.5%. Analysis condition B: Retention time=1.64 min; ESI-MS(+) m/z [M+2H]²⁺: 934.1.

### Preparation of Compound 1899

Compound 1899 was prepared on a 50 μmol scale. The yield of the product was 48.7 mg, and its estimated purity by LCMS analysis was 97.5%. Analysis condition A: Retention time=1.62 min; ESI-MS(+) m/z [M+2H]²⁺: 939.1.

### Preparation of Compound 1900

Compound 1900 was prepared on a 50 μmol scale. The yield of the product was 19.2 mg, and its estimated purity by LCMS analysis was 98%. Analysis condition B: Retention time=1.54 min; ESI-MS(+) m/z [M+H]⁺: 1836.

### Preparation of Compound 1901

Compound 1901 was prepared on a 50 μmol scale. The yield of the product was 31.5 mg, and its estimated purity by LCMS analysis was 97.8%. Analysis condition B: Retention time=1.77 min; ESI-MS(+) m/z [M+2H]²⁺: 1010.3.

### Preparation of Compound 1902

Compound 1902 was prepared on a 50 μmol scale. The yield of the product was 32.8 mg, and its estimated purity by LCMS analysis was 97.3%. Analysis condition B: Retention time=1.75 min; ESI-MS(+) m/z [M+2H]²⁺: 1017.2.

### Preparation of Compound 1903

Compound 1903 was prepared on a 50 μmol scale. The yield of the product was 53.8 mg, and its estimated purity by LCMS analysis was 95.7%. Analysis condition B: Retention time=1.79 min; ESI-MS(+) m/z [M+2H]²⁺: 1017.

### Preparation of Compound 1904

Compound 1904 was prepared on a 50 μmol scale. The yield of the product was 30.2 mg, and its estimated purity by LCMS analysis was 95.6%. Analysis condition B: Retention time=1.68 min; ESI-MS(+) m/z [M+2H]²⁺: 1010.2.

### Preparation of Compound 1905

Compound 1905 was prepared on a 50 μmol scale. The yield of the product was 12.6 mg, and its estimated purity by LCMS analysis was 92.9%. Analysis condition B: Retention time=1.6 min; ESI-MS(+) m/z [M+2H]²⁺: 1003.2.

### Preparation of Compound 1906

Compound 1906 was prepared on a 50 μmol scale. The yield of the product was 42.6 mg, and its estimated purity by LCMS analysis was 99.4%. Analysis condition B: Retention time=1.71 min; ESI-MS(+) m/z [M+2H]²⁺: 1042.2.

### Preparation of Compound 1907

Compound 1907 was prepared on a 50 μmol scale. The yield of the product was 44.5 mg, and its estimated purity by LCMS analysis was 96.6%. Analysis condition B: Retention time=1.72 min; ESI-MS(+) m/z [M+2H]²⁺: 1042.1.

### Preparation of Compound 1908

Compound 1908 was prepared on a 50 μmol scale. The yield of the product was 36.3 mg, and its estimated purity by LCMS analysis was 91.9%. Analysis condition A: Retention time=1.72 min; ESI-MS(+) m/z [M+2H]²⁺: 1035.

### Preparation of Compound 1909

Compound 1909 was prepared on a 50 μmol scale. The yield of the product was 14.9 mg, and its estimated purity by LCMS analysis was 95.5%. Analysis condition A: Retention time=1.51 min; ESI-MS(+) m/z [M+2H]²⁺: 1024.1.

### Preparation of Compound 1910

Compound 1910 was prepared on a 50 μmol scale. The yield of the product was 12.2 mg, and its estimated purity by LCMS analysis was 98.300. Analysis condition A: Retention time=1.52 min; ESI-MS(+) m/z [M+2H]²⁺: 1032.1.

### Preparation of Compound 1911

Compound 1911 was prepared on a 50 μmol scale. The yield of the product was 34.3 mg, and its estimated purity by LCMS analysis was 95.1%. Analysis condition A: Retention time=1.66 min; ESI-MS(+) m/z [M+2H]²⁺: 1032.1.

### Preparation of Compound 1912

Compound 1912 was prepared on a 50 μmol scale. The yield of the product was 25 mg, and its estimated purity by LCMS analysis was 97.6%. Analysis condition B: Retention time=1.92 min; ESI-MS(+) m/z [M+2H]²⁺: 1031.1.

### Preparation of Compound 1913

Compound 1913 was prepared on a 50 μmol scale. The yield of the product was 19.9 mg, and its estimated purity by LCMS analysis was 96.5%. Analysis condition B: Retention time=1.86 min; ESI-MS(+) m/z [M+2H]²⁺: 1018.2.

### Preparation of Compound 1914

Compound 1914 was prepared on a 50 μmol scale. The yield of the product was 24.5 mg, and its estimated purity by LCMS analysis was 92%. Analysis condition A: Retention time=1.59 min; ESI-MS(+) m/z [M+2H]²⁺: 1038.1.

### Preparation of Compound 1915

Compound 1915 was prepared on a 50 μmol scale. The yield of the product was 30 mg, and its estimated purity by LCMS analysis was 94.7%. Analysis condition A: Retention time=1.68 min; ESI-MS(+) m/z [M+2H]²⁺: 1039.

### Preparation of Compound 1916

Compound 1916 was prepared on a 50 μmol scale. The yield of the product was 22.6 mg, and its estimated purity by LCMS analysis was 99.4%. Analysis condition A: Retention time=1.68 min; ESI-MS(+) m/z [M+2H]²⁺: 1046.1.

### Preparation of Compound 1917

Compound 1917 was prepared on a 50 μmol scale. The yield of the product was 16.6 mg, and its estimated purity by LCMS analysis was 96.5%. Analysis condition A: Retention time=1.54 min; ESI-MS(+) m/z [M+2H]²⁺: 1038.1.

### Preparation of Compound 1918

Compound 1918 was prepared on a 50 μmol scale. The yield of the product was 2.7 mg, and its estimated purity by LCMS analysis was 91%. Analysis condition B: Retention time=1.69 min; ESI-MS(+) m/z [M+2H]²⁺: 1042.1.

### Preparation of Compound 1919

Compound 1919 was prepared on a 50 μmol scale. The yield of the product was 19.2 mg, and its estimated purity by LCMS analysis was 90.4%. Analysis condition A: Retention time=1.75 min; ESI-MS(+) m/z [M+2H]²⁺: 1045.1.

### Preparation of Compound 1920

Compound 1920 was prepared on a 50 μmol scale. The yield of the product was 17.5 mg, and its estimated purity by LCMS analysis was 93.8%. Analysis condition A: Retention time=1.64 min; ESI-MS(+) m/z [M+2H]²⁺: 1059.

### Preparation of Compound 1921

Compound 1921 was prepared on a 50 μmol scale. The yield of the product was 19 mg, and its estimated purity by LCMS analysis was 94.4%. Analysis condition B: Retention time=1.91 min; ESI-MS(+) m/z [M+2H]²⁺: 1059.

### Preparation of Compound 1922

Compound 1922 was prepared on a 50 μmol scale. The yield of the product was 18.4 mg, and its estimated purity by LCMS analysis was 96.1%. Analysis condition B: Retention time=1.94 min; ESI-MS(+) m/z [M+2H]²⁺: 1045.2.

### Preparation of Compound 1923

Compound 1923 was prepared on a 50 μmol scale. The yield of the product was 15.2 mg, and its estimated purity by LCMS analysis was 95.5%. Analysis condition B: Retention time=1.92 min; ESI-MS(+) m/z [M+2H]²⁺: 1038.1.

### Preparation of Compound 1924

Compound 1924 was prepared on a 50 μmol scale. The yield of the product was 14.8 mg, and its estimated purity by LCMS analysis was 95.4%. Analysis condition B: Retention time=1.92 min; ESI-MS(+) m/z [M+2H]²⁺: 1059.2.

### Preparation of Compound 1925

Compound 1925 was prepared on a 50 μmol scale. The yield of the product was 19.7 mg, and its estimated purity by LCMS analysis was 97.8%. Analysis condition B: Retention time=1.92 min; ESI-MS(+) m/z [M+2H]²⁺: 1059.1.

### Preparation of Compound 1926

Compound 1926 was prepared on a 50 μmol scale. The yield of the product was 5 mg, and its estimated purity by LCMS analysis was 94%. Analysis condition B: Retention time=1.87 min; ESI-MS(+) m/z [M+2H]²⁺: 1038.3.

### Preparation of Compound 1927

Compound 1927 was prepared on a 50 μmol scale. The yield of the product was 3.8 mg, and its estimated purity by LCMS analysis was 97.9%. Analysis condition B: Retention time=1.84 min; ESI-MS(+) m/z [M+2H]²⁺: 1052.1.

### Preparation of Compound 1928

Compound 1928 was prepared on a 50 μmol scale. The yield of the product was 18.3 mg, and its estimated purity by LCMS analysis was 94.1%. Analysis condition A: Retention time=1.77 min; ESI-MS(+) m/z [M+2H]²⁺: 1032.1.

### Preparation of Compound 1929

Compound 1929 was prepared on a 50 μmol scale. The yield of the product was 7.7 mg, and its estimated purity by LCMS analysis was 99.1%. Analysis condition A: Retention time=1.72 min; ESI-MS(+) m/z [M+2H]²⁺: 1025.1.

### Preparation of Compound 1930

Compound 1930 was prepared on a 50 μmol scale. The yield of the product was 17.5 mg, and its estimated purity by LCMS analysis was 96.7%. Analysis condition A: Retention time=1.65 min; ESI-MS(+) m/z [M+2H]²⁺: 1046.1.

### Preparation of Compound 1931

Compound 1931 was prepared on a 50 μmol scale. The yield of the product was 3.1 mg, and its estimated purity by LCMS analysis was 95%. Analysis condition B: Retention time=1.89 min; ESI-MS(+) m/z [M+2H]²⁺: 1045.1.

### Preparation of Compound 1932

Compound 1932 was prepared on a 50 μmol scale. The yield of the product was 25 mg, and its estimated purity by LCMS analysis was 97.9%. Analysis condition B: Retention time=1.84 min; ESI-MS(+) m/z [M+2H]²⁺: 1051.1.

### Preparation of Compound 1933

Compound 1933 was prepared on a 50 μmol scale. The yield of the product was 24.5 mg, and its estimated purity by LCMS analysis was 100%. Analysis condition A: Retention time=1.66 min; ESI-MS(+) m/z [M+2H]²⁺: 1058.1.

### Preparation of Compound 1934

Compound 1934 was prepared on a 50 μmol scale. The yield of the product was 14.7 mg, and its estimated purity by LCMS analysis was 96.4%. Analysis condition A: Retention time=1.57 min; ESI-MS(+) m/z [M+2H]²⁺: 1051.

### Preparation of Compound 1935

Compound 1935 was prepared on a 50 μmol scale. The yield of the product was 20.9 mg, and its estimated purity by LCMS analysis was 94.9%. Analysis condition A: Retention time=1.64 min; ESI-MS(+) m/z [M+2H]²⁺: 1059.2.

### Preparation of Compound 1936

Compound 1936 was prepared on a 50 μmol scale. The yield of the product was 2.5 mg, and its estimated purity by LCMS analysis was 95%. Analysis condition A: Retention time=1.31 min; ESI-MS(+) m/z [M+2H]²⁺: 1111.1.

### Preparation of Compound 1937

Compound 1937 was prepared on a 50 μmol scale. The yield of the product was 24 mg, and its estimated purity by LCMS analysis was 93.1%. Analysis condition B: Retention time=1.82 min; ESI-MS(+) m/z [M+2H]²⁺: 1159.

### Preparation of Compound 1938

Compound 1938 was prepared on a 50 μmol scale. The yield of the product was 16.6 mg, and its estimated purity by LCMS analysis was 95.4%. Analysis condition A: Retention time=1.26 min; ESI-MS(+) m/z [M+2H]²⁺: 1137.

### Preparation of Compound 1939

Compound 1939 was prepared on a 50 μmol scale. The yield of the product was 45.8 mg, and its estimated purity by LCMS analysis was 98.3%. Analysis condition A: Retention time=1.4 min; ESI-MS(+) m/z [M+2H]²⁺: 1137.

### Preparation of Compound 1940

Compound 1940 was prepared on a 50 μmol scale. The yield of the product was 16 mg, and its estimated purity by LCMS analysis was 95.9%. Analysis condition B: Retention time=1.83 min; ESI-MS(+) m/z [M+2H]²⁺: 1064.9.

### Preparation of Compound 1941

Compound 1941 was prepared on a 50 μmol scale. The yield of the product was 18.5 mg, and its estimated purity by LCMS analysis was 97.2%. Analysis condition B: Retention time=1.98 min; ESI-MS(+) m/z [M+2H]²⁺: 1066.1.

### Preparation of Compound 1942

Compound 1942 was prepared on a 50 μmol scale. The yield of the product was 14 mg, and its estimated purity by LCMS analysis was 96.3%. Analysis condition B: Retention time=1.77 min; ESI-MS(+) m/z [M+2H]²⁺: 1031.

### Preparation of Compound 1943

Compound 1943 was prepared on a 50 μmol scale. The yield of the product was 23.6 mg, and its estimated purity by LCMS analysis was 96.1%. Analysis condition A: Retention time=1.78 min; ESI-MS(+) m/z [M+2H]²⁺: 1032.1.

### Preparation of Compound 1944

Compound 1944 was prepared on a 50 μmol scale. The yield of the product was 38.9 mg, and its estimated purity by LCMS analysis was 95.8%. Analysis condition B: Retention time=1.82 min; ESI-MS(+) m/z [M+2H]²⁺: 1144.

### Preparation of Compound 1945

Compound 1945 was prepared on a 50 μmol scale. The yield of the product was 35.6 mg, and its estimated purity by LCMS analysis was 100%. Analysis condition A: Retention time=1.35 min; ESI-MS(+) m/z [M+2H]²⁺: 1144.1.

### Preparation of Compound 1946

Compound 1946 was prepared on a 50 μmol scale. The yield of the product was 48.9 mg, and its estimated purity by LCMS analysis was 100%. Analysis condition A: Retention time=1.37 min; ESI-MS(+) m/z [M+2H]²⁺: 1129.1.

### Preparation of Compound 1947

Compound 1947 was prepared on a 50 μmol scale. The yield of the product was 25.9 mg, and its estimated purity by LCMS analysis was 95.7%. Analysis condition B: Retention time=1.76 min; ESI-MS(+) m/z [M+2H]²⁺: 1114.3.

### Preparation of Compound 1948

To a 45-mL polypropylene solid-phase reaction vessel was added Rink resin (100 mg, 0.050 mmol), and the reaction vessel was placed on the Symphony peptide synthesizer. The following procedures were then performed sequentially: “Symphony Resin-swelling procedure” was followed; “Symphony Single-coupling procedure” was followed with Fmoc-Dab-OH; “Symphony Single-coupling procedure” was followed with Fmoc-Cys(Trt)-OH; “Symphony Single-coupling procedure” was followed with Fmoc-Orn(Boc)-OH; “Symphony Single-coupling procedure” was followed with Fmoc-Val-OH; “Symphony Single-coupling procedure” was followed with Fmoc-Cha-OH; “Symphony Single-coupling procedure” was followed with Fmoc-Dab(Boc)-OH; “Symphony Single-coupling procedure” was followed with Fmoc-D-Leu-OH; “Symphony Single-coupling procedure” or “Symphony X double-coupling procedure” was followed with Fmoc-N-Me-Ala-OH; “Symphony Single-coupling procedure” was followed with Fmoc-Val-OH; “Symphony Single-coupling procedure” was followed with Fmoc-Bip-OH; “Symphony Single-coupling procedure” was followed with Fmoc-Leu-OH; “Symphony Single-coupling procedure” was followed with Fmoc-Trp(Boc)-OH; “Symphony Single-coupling procedure” was followed with Fmoc-Asp(tBu)-OH; “Symphony Single-coupling procedure” was followed with Fmoc-4-Pya-OH (Fmoc-Ala(β-4-pyridinyl)-OH); “Symphony Single-coupling procedure” was followed with Fmoc-Tyr(CH₂CO₂tBu)-OH; “Symphony Chloroacetic Anhydride coupling procedure” was followed; “Symphony Final rinse and dry procedure” was followed; “Global Deprotection Method A” was followed; “Cyclization Method A” was followed.

The crude material was purified via preparative LC/MS with the following conditions: Column: XBridge C18, 200 mm×19 mm, 5-μm particles; Mobile Phase A: 5:95 acetonitrile:water with 10 mM ammonium acetate; Mobile Phase B: 95:5 acetonitrile:water with 10-mM ammonium acetate; Gradient: a 0-minute hold at 19% B, 19-59% B over 20 minutes, then a 4-minute hold at 100% B; Flow Rate: 20 mL/min; Column Temperature: 25 C. Fraction collection was triggered by MS signals. Fractions containing the desired product were combined and dried via centrifugal evaporation. The yield of the product was 8.8 mg, and its estimated purity by LCMS analysis was 93.8%.

Analysis condition A: Retention time=1.56 min; ESI-MS(+) m/z [M+3H]³⁺: 678.

### Preparation of Compound 2000

Compound 2000 was prepare on a 50 μmol scale. The yield of the product was 38.4 mg, and its estimated purity by LCMS analysis was 95.5%. Analysis condition B: Retention time=1.71 min; ESI-MS(+) m/z [M+2H]²⁺: 1018.2.

### Preparation of Compound-2001

Compound 2001 was prepared on a 50 μmol scale. The yield of the product was 36.1 mg, and its estimated purity by LCMS analysis was 97%. Analysis condition A: Retention time=1.71 min; ESI-MS(+) m/z [M+2H]²⁺: 1046.

### Preparation of Compound 2002

Compound 2002 was prepared on a 50 μmol scale. The yield of the product was 36 mg, and its estimated purity by LCMS analysis was 95.2%. Analysis condition B: Retention time=1.89 min; ESI-MS(+) m/z [M+2H]²⁺: 1028.8.

### Preparation of Compound 2003

Compound 2003 was prepared on a 50 μmol scale. The yield of the product was 53.7 mg, and its estimated purity by LCMS analysis was 87.8%. Analysis condition B: Retention time=1.97 min; ESI-MS(+) m/z [M+H]⁺: 1998.

### Preparation of Compound 2004

Compound 2004 was prepared on a 50 μmol scale. The yield of the product was 43.5 mg, and its estimated purity by LCMS analysis was 97.6%. Analysis condition B: Retention time=1.62 min; ESI-MS(+) m/z [M+3H]³⁺: 675.1.

### Preparation of Compound 2005

Compound 2005 was prepared on a 50 μmol scale. The yield of the product was 11.9 mg, and its estimated purity by LCMS analysis was 95.7%. Analysis condition A: Retention time=1.56 min; ESI-MS(+) m/z [M+2H]²⁺: 1060.3.

### Preparation of Compound 2006

Compound 2006 was prepared on a 50 μmol scale. The yield of the product was 24.3 mg, and its estimated purity by LCMS analysis was 96.4%. Analysis condition A: Retention time=1.88 min; ESI-MS(+) m/z [M+2H]²⁺: 1017.1.

### Preparation of Compound 2007

Compound 2007 was prepared on a 50 μmol scale. The yield of the product was 15.6 mg, and its estimated purity by LCMS analysis was 100%. Analysis condition A: Retention time=1.61 min; ESI-MS(+) m/z [M+2H]²⁺: 1055.

### Preparation of Compound 2008

Compound 2008 was prepared on a 50 μmol scale. The yield of the product was 2.2 mg, and its estimated purity by LCMS analysis was 96.5%. Analysis condition B: Retention time=1.96 min; ESI-MS(+) m/z [M+2H]²⁺: 1095.9.

### Preparation of Compound 2009

Compound 2009 was prepared on a 50 μmol scale. The yield of the product was 11.8 mg, and its estimated purity by LCMS analysis was 97.7%. Analysis condition A: Retention time=1.54 min; ESI-MS(+) m/z [M+3H]³⁺: 653.4.

### Preparation of Compound 2010

Compound 2010 was prepared on a 50 μmol scale. The yield of the product was 55.1 mg, and its estimated purity by LCMS analysis was 96.6%. Analysis condition B: Retention time=1.64 min; ESI-MS(+) m/z [M+2H]²⁺: 1124.6.

### Preparation of Compound 2011

Compound 2011 was prepared on a 50 μmol scale. The yield of the product was 26.7 mg, and its estimated purity by LCMS analysis was 100%. Analysis condition A: Retention time=1.58 min; ESI-MS(+) m/z [M+2H]²⁺: 1031.9.

### Preparation of Compound 2012

Compound 2012 was prepared on a 50 μmol scale. The yield of the product was 30.3 mg, and its estimated purity by LCMS analysis was 100%. Analysis condition A: Retention time=1.73 min; ESI-MS(+) m/z [M+2H]²⁺: 1010.

### Preparation of Compound 2013

Compound 2013 was prepared on a 50 μmol scale. The yield of the product was 42.1 mg, and its estimated purity by LCMS analysis was 95.1%. Analysis condition B: Retention time=1.91 min; ESI-MS(+) m/z [M+2H]²⁺: 1038.2.

### Preparation of Compound 2014

Compound 2014 was prepared on a 50 μmol scale. The yield of the product was 31.7 mg, and its estimated purity by LCMS analysis was 97.6%. Analysis condition B: Retention time=1.89 min; ESI-MS(+) m/z [M+2H]²⁺: 1031.

### Preparation of Compound 2015

Compound 2015 was prepared on a 50 μmol scale. The yield of the product was 38.7 mg, and its estimated purity by LCMS analysis was 97.3%. Analysis condition B: Retention time=1.9 min; ESI-MS(+) m/z [M+2H]²⁺: 1059.8.

### Preparation of Compound 2016

Compound 2016 was prepared on a 50 μmol scale. The yield of the product was 9.5 mg, and its estimated purity by LCMS analysis was 98.3%. Analysis condition A: Retention time=1.48 min; ESI-MS(+) m/z [M+2H]²⁺: 1089.

### Preparation of Compound 2017

Compound 2017 was prepared on a 50 μmol scale. The yield of the product was 9.7 mg, and its estimated purity by LCMS analysis was 97%. Analysis condition A: Retention time=1.55 min; ESI-MS(+) m/z [M+2H]²⁺: 1060.

### Preparation of Compound 2018

Compound 2018 was prepared on a 50 μmol scale. The yield of the product was 24.6 mg, and its estimated purity by LCMS analysis was 95%. Analysis condition A: Retention time=1.73 min; ESI-MS(+) m/z [M+2H]²⁺: 1066.2.

### Preparation of Compound 2019

Compound 2019 was prepared on a 50 μmol scale. The yield of the product was 51.1 mg, and its estimated purity by LCMS analysis was 98.1%. Analysis condition B: Retention time=2 min; ESI-MS(+) m/z [M+2H]²⁺: 1088.9.

### Preparation of Compound 2020

Compound 2020 was prepared on a 50 μmol scale. The yield of the product was 25.6 mg, and its estimated purity by LCMS analysis was 91.6%. Analysis condition B: Retention time=2 min; ESI-MS(+) m/z [M+2H]²⁺: 1074.2.

### Preparation of Compound 2021

Compound 2021 was prepared on a 50 μmol scale. The yield of the product was 6.5 mg, and its estimated purity by LCMS analysis was 90.9%. Analysis condition B: Retention time=1.65 min; ESI-MS(+) m/z [M+2H]²⁺: 958.1.

### Preparation of Compound 2022

Compound 2022 was prepared on a 50 μmol scale. The yield of the product was 36.5 mg, and its estimated purity by LCMS analysis was 95.8%. Analysis condition A: Retention time=1.67 min; ESI-MS(+) m/z [M+2H]²⁺: 1017.2.

### Preparation of Compound 2023

Compound 2023 was prepared on a 50 μmol scale. The yield of the product was 44.2 mg, and its estimated purity by LCMS analysis was 97.8%. Analysis condition A: Retention time=1.55 min; ESI-MS(+) m/z [M+2H]²⁺: 1003.1.

### Preparation of Compound 2024

Compound 2024 was prepared on a 50 μmol scale. The yield of the product was 42.5 mg, and its estimated purity by LCMS analysis was 97.7%. Analysis condition B: Retention time=1.84 min; ESI-MS(+) m/z [M+H]⁺: 1901.6.

### Preparation of Compound 2025

Compound 2025 was prepared on a 50 μmol scale. The yield of the product was 15.8 mg, and its estimated purity by LCMS analysis was 96.5%. Analysis condition A: Retention time=1.83 min; ESI-MS(+) m/z [M+H]⁺: 1878.2.

### Preparation of Compound 2026

Compound 2026 was prepared on a 50 μmol scale. The yield of the product was 22.7 mg, and its estimated purity by LCMS analysis was 96%. Analysis condition A: Retention time=1.78 min; ESI-MS(+) m/z [M+H]⁺: 1893.2.

### Preparation of Compound 2027

Compound 2027 was prepared on a 50 μmol scale. The yield of the product was 23.6 mg, and its estimated purity by LCMS analysis was 96.6%. Analysis condition B: Retention time=1.91 min; ESI-MS(+) m/z [M+2H]²⁺: 976.0

### Preparation of Compound 2028

Compound 2028 was prepared on a 50 μmol scale. The yield of the product was 23.9 mg, and its estimated purity by LCMS analysis was 95.7%. Analysis condition B: Retention time=1.88 min; ESI-MS(+) m/z [M+2H]²⁺: 990.1.

### Preparation of Compound 2029

Compound 2029 was prepared on a 50 μmol scale. The yield of the product was 56.4 mg, and its estimated purity by LCMS analysis was 96.7%. Analysis condition B: Retention time=1.85 min; ESI-MS(+) m/z [M+2H]²⁺: 983.2.

### Preparation of Compound 2030

Compound 2030 was prepared on a 50 μmol scale. The yield of the product was 34.8 mg, and its estimated purity by LCMS analysis was 97.5%. Analysis condition B: Retention time=1.87 min; ESI-MS(+) m/z [M+H]⁺: 1976.9.

### Preparation of Compound 2031

Compound 2031 was prepared on a 50 μmol scale. The yield of the product was 48.8 mg, and its estimated purity by LCMS analysis was 100%. Analysis condition A: Retention time=1.68, 1.72 min; ESI-MS(+) m/z [M+H]⁺: 1948.14, 1948.14.

### Preparation of Compound 2032

Compound 2032 was prepared on a 50 μmol scale. The yield of the product was 21.5 mg, and its estimated purity by LCMS analysis was 100%. Analysis condition A: Retention time=1.7 min; ESI-MS(+) m/z [M+H]⁺: 1936.1.

### Preparation of Compound 2033

Compound 2033 was prepared on a 50 μmol scale. The yield of the product was 41.5 mg, and its estimated purity by LCMS analysis was 100%. Analysis condition A: Retention time=1.72 min; ESI-MS(+) m/z [M+H]⁺: 1934.1.

### Preparation of Compound 2034

Compound 2034 was prepared on a 50 μmol scale. The yield of the product was 46.6 mg, and its estimated purity by LCMS analysis was 91.9%. Analysis condition B: Retention time=1.58 min; ESI-MS(+) m/z [M+H]⁺: 1887.

### Preparation of Compound 2035

Compound 2035 was prepared on a 50 μmol scale. The yield of the product was 36.1 mg, and its estimated purity by LCMS analysis was 92.2%. Analysis condition B: Retention time=1.57 min; ESI-MS(+) m/z [M+H]⁺: 1985.6.

### Preparation of Compound 2036

Compound 2036 was prepared on a 50 μmol scale. The yield of the product was 31.4 mg, and its estimated purity by LCMS analysis was 95.6%. Analysis condition A: Retention time=1.68 min; ESI-MS(+) m/z [M+H]⁺: 1985.

### Preparation of Compound 2037

Compound 2037 was prepared on a 50 μmol scale. The yield of the product was 20.6 mg, and its estimated purity by LCMS analysis was 100%. Analysis condition A: Retention time=1.55 min; ESI-MS(+) m/z [M+2H]²⁺: 993.2.

### Preparation of Compound 2038

Compound 2038 was prepared on a 50 μmol scale. The yield of the product was 44.3 mg, and its estimated purity by LCMS analysis was 95.3%. Analysis condition B: Retention time=1.59 min; ESI-MS(+) m/z [M+H]⁺: 1969.1.

### Preparation of Compound 2039

Compound 2039 was prepared on a 50 μmol scale. The yield of the product was 27.8 mg, and its estimated purity by LCMS analysis was 97.1%. Analysis condition B: Retention time=1.55 min; ESI-MS(+) m/z [M+2H]²⁺: 951.6.

### Preparation of Compound 2040

Compound 2040 was prepared on a 50 μmol scale. The yield of the product was 41.6 mg, and its estimated purity by LCMS analysis was 100%. Analysis condition A: Retention time=1.77 min; ESI-MS(+) m/z [M+2H]²⁺: 929.

### Preparation of Compound 2041

Compound 2041 was prepared on a 50 μmol scale. The yield of the product was 54.6 mg, and its estimated purity by LCMS analysis was 92.7%. Analysis condition B: Retention time=1.39 min; ESI-MS(+) m/z [M+2H]²⁺: 915.1.

### Preparation of Compound 2042

Compound 2042 was prepared on a 50 μmol scale. The yield of the product was 28.5 mg, and its estimated purity by LCMS analysis was 100%. Analysis condition A: Retention time=1.82 min; ESI-MS(+) m/z [M+H]⁺: 1930.1.

### Preparation of Compound 2043

Compound 2043 was prepared on a 50 μmol scale. The yield of the product was 10.9 mg, and its estimated purity by LCMS analysis was 96.7%. Analysis condition B: Retention time=1.59 min; ESI-MS(+) m/z [M+3H]³⁺: 647.1.

### Preparation of Compound 2044

Compound 2044 was prepared on a 50 μmol scale. The yield of the product was 18.3 mg, and its estimated purity by LCMS analysis was 99.3%. Analysis condition B: Retention time=1.6 min; ESI-MS(+) m/z [M+3H]³⁺: 661.3.

### Preparation of Compound 2045

Compound 2045 was prepared on a 50 μmol scale. The yield of the product was 14.7 mg, and its estimated purity by LCMS analysis was 98.5%. Analysis condition A: Retention time=1.58 min; ESI-MS(+) m/z [M+3H]³⁺: 661.0.

### Preparation of Compound 2046

Compound 2046 was prepared on a 50 μmol scale. The yield of the product was 18.6 mg, and its estimated purity by LCMS analysis was 100%. Analysis condition A: Retention time=1.75 min; ESI-MS(+) m/z [M+2H]²⁺: 990.9.

### Preparation of Compound 2047

Compound 2047 was prepared on a 50 μmol scale. The yield of the product was 52.4 mg, and its estimated purity by LCMS analysis was 95.3%. Analysis condition B: Retention time=1.98 min; ESI-MS(+) m/z [M+H]⁺: 1972.9.

### Preparation of Compound 2048

Compound 2048 was prepared on a 50 μmol scale. The yield of the product was 90.7 mg, and its estimated purity by LCMS analysis was 96.9%. Analysis condition B: Retention time=1.72 min; ESI-MS(+) m/z [M+2H]²⁺: 1005.

### Preparation of Compound 2049

Compound 2049 was prepared on a 50 μmol scale. The yield of the product was 54.3 mg, and its estimated purity by LCMS analysis was 98.4%. Analysis condition B: Retention time=1.65 min; ESI-MS(+) m/z [M+2H]²⁺: 1004.8.

### Preparation of Compound 2050

Compound 2050 was prepared on a 50 μmol scale. The yield of the product was 26.3 mg, and its estimated purity by LCMS analysis was 98.4%. Analysis condition A: Retention time=1.78 min; ESI-MS(+) m/z [M+H]⁺: 1883.3.

### Preparation of Compound 2051

Compound 2051 was prepared on a 50 μmol scale. The yield of the product was 8.8 mg, and its estimated purity by LCMS analysis was 96.8%. Analysis condition A: Retention time=1.8 min; ESI-MS(+) m/z [M+H]⁺: 1937.9.

### Preparation of Compound 2052

Compound 2052 was prepared on a 50 μmol scale. The yield of the product was 41.3 mg, and its estimated purity by LCMS analysis was 93%. Analysis condition B: Retention time=1.74 min; ESI-MS(+) m/z [M+H]⁺: 1931.3.

### Preparation of Compound 2053

Compound 2053 was prepared on a 50 μmol scale. The yield of the product was 9.6 mg, and its estimated purity by LCMS analysis was 94.9%. Analysis condition B: Retention time=1.91 min; ESI-MS(+) m/z [M+H]⁺: 1969.4.

### Preparation of Compound 2054

Compound 2054 was prepared on a 50 μmol scale. The yield of the product was 23 mg, and its estimated purity by LCMS analysis was 99.3%. Analysis condition A: Retention time=1.77 min; ESI-MS(+) m/z [M+H]⁺: 1965.3.

### Preparation of Compound 2055

Compound 2055 was prepared on a 50 μmol scale. The yield of the product was 45.4 mg, and its estimated purity by LCMS analysis was 98.5%. Analysis condition B: Retention time=1.6 min; ESI-MS(+) m/z [M+2H]²⁺: 1010.8.

### Preparation of Compound 2056

Compound 2056 was prepared on a 50 μmol scale. The yield of the product was 71.4 mg, and its estimated purity by LCMS analysis was 98.5%. Analysis condition A: Retention time=1.55 min; ESI-MS(+) m/z [M+2H]²⁺: 953.4.

### Preparation of Compound 2057

Compound 2057 was prepared on a 50 μmol scale. The yield of the product was 50 mg, and its estimated purity by LCMS analysis was 98.2%. Analysis condition B: Retention time=1.52 min; ESI-MS(+) m/z [M+H]⁺: 1935.2.

### Preparation of Compound 2058

Compound 2058 was prepared on a 50 μmol scale. The yield of the product was 33.6 mg, and its estimated purity by LCMS analysis was 97.2%. Analysis condition A: Retention time=1.55 min; ESI-MS(+) m/z [M+H]⁺: 1935.3.

### Preparation of Compound 2059

Compound 2059 was prepared on a 50 μmol scale. The yield of the product was 13.6 mg, and its estimated purity by LCMS analysis was 100%. Analysis condition B: Retention time=1.61 min; ESI-MS(+) m/z [M+2H]²⁺: 967.8.

### Preparation of Compound 2060

Compound 2060 was prepared on a 50 μmol scale. The yield of the product was 22.5 mg, and its estimated purity by LCMS analysis was 95.5%. Analysis condition A: Retention time=1.85 min; ESI-MS(+) m/z [M+H]⁺: 1905.8.

### Preparation of Compound 2061

Compound 2061 was prepared on a 50 μmol scale. The yield of the product was 44.2 mg, and its estimated purity by LCMS analysis was 95.6%. Analysis condition A: Retention time=1.67 min; ESI-MS(+) m/z [M+2H]²⁺: 949.

### Preparation of Compound 2062

Compound 2062 was prepared on a 50 μmol scale. The yield of the product was 43.3 mg, and its estimated purity by LCMS analysis was 97.7%. Analysis condition B: Retention time=1.72 min; ESI-MS(+) m/z [M+2H]²⁺: 980.

### Preparation of Compound 2063

Compound 2063 was prepared on a 50 μmol scale. The yield of the product was 7.9 mg, and its estimated purity by LCMS analysis was 98%. Analysis condition A: Retention time=1.57 min; ESI-MS(+) m/z [M+H]⁺: 1918.

### Preparation of Compound 2064

Compound 2064 was prepared on a 50 μmol scale. The yield of the product was 5.1 mg, and its estimated purity by LCMS analysis was 95.9%. Analysis condition A: Retention time=1.57 min; ESI-MS(+) m/z [M+H]⁺: 1932.8.

### Preparation of Compound 2065

Compound 2065 was prepared on a 50 μmol scale. The yield of the product was 52.1 mg, and its estimated purity by LCMS analysis was 97.5%. Analysis condition A: Retention time=1.63 min; ESI-MS(+) m/z [M+2H]²⁺: 948.1.

### Preparation of Compound 2066

Compound 2066 was prepared on a 50 μmol scale. The yield of the product was 32.5 mg, and its estimated purity by LCMS analysis was 100%. Analysis condition A: Retention time=1.97 min; ESI-MS(+) m/z [M+H]⁺: 1896.3.

### Preparation of Compound 2067

Compound 2067 was prepared on a 50 μmol scale. The yield of the product was 46.4 mg, and its estimated purity by LCMS analysis was 95.3%. Analysis condition B: Retention time=1.92, 2.09 min; ESI-MS(+) m/z [M+2H]²⁺: 1038.21, 1038.34.

### Preparation of Compound 2068

Compound 2068 was prepared on a 50 μmol scale. The yield of the product was 16 mg, and its estimated purity by LCMS analysis was 99.4%. Analysis condition B: Retention time=1.89 min; ESI-MS(+) m/z [M+2H]²⁺: 1017.1.

### Preparation of Compound 2069

Compound 2069 was prepared on a 50 μmol scale. The yield of the product was 40.5 mg, and its estimated purity by LCMS analysis was 97.1%. Analysis condition A: Retention time=1.47 min; ESI-MS(+) m/z [M+2H]²⁺: 951.2.

### Preparation of Compound 2070

Compound 2070 was prepared on a 50 μmol scale. The yield of the product was 32.3 mg, and its estimated purity by LCMS analysis was 98.4%. Analysis condition A: Retention time=1.57 min; ESI-MS(+) m/z [M+H]⁺: 1843.2.

### Preparation of Compound 2071

Compound 2071 was prepared on a 50 μmol scale. The yield of the product was 30.3 mg, and its estimated purity by LCMS analysis was 98.9%. Analysis condition A: Retention time=1.67 min; ESI-MS(+) m/z [M+H]⁺: 1872.2.

### Preparation of Compound 2072

Compound 2072 was prepared on a 50 μmol scale. The yield of the product was 56.4 mg, and its estimated purity by LCMS analysis was 95.9%. Analysis condition A: Retention time=1.85 min; ESI-MS(+) m/z [M+H]⁺: 1911.1.

### Preparation of Compound 2073

Compound 2073 was prepared on a 50 μmol scale. The yield of the product was 77.4 mg, and its estimated purity by LCMS analysis was 98.2%. Analysis condition A: Retention time=1.73 min; ESI-MS(+) m/z [M+2H]²⁺: 949.7.

### Preparation of Compound 2074

Compound 2074 was prepared on a 25 μmol scale. The yield of the product was 16.1 mg, and its estimated purity by LCMS analysis was 98.2%. Analysis condition A: Retention time=1.58 min; ESI-MS(+) m/z [M+H]⁺: 1885.9.

### Preparation of Compound 2075

Compound 2075 was prepared on a 25 μmol scale. The yield of the product was 8 mg, and its estimated purity by LCMS analysis was 96.9%. Analysis condition A: Retention time=1.64 min; ESI-MS(+) m/z [M+H]⁺: 1873.2.

### Preparation of Compound 2076

Compound 2076 was prepared on a 25 μmol scale. The yield of the product was 12.9 mg, and its estimated purity by LCMS analysis was 98.4%. Analysis condition B: Retention time=1.8 min; ESI-MS(+) m/z [M+H]⁺: 1901.2.

### Preparation of Compound 2077

Compound 2077 was prepared on a 50 μmol scale. The yield of the product was 23.7 mg, and its estimated purity by LCMS analysis was 95.7%. Analysis condition B: Retention time=1.82 min; ESI-MS(+) m/z [M+2H]²⁺: 1018.2.

### Preparation of Compound 2078

Compound 2078 was prepared on a 50 μmol scale. The yield of the product was 30.4 mg, and its estimated purity by LCMS analysis was 95.2%. Analysis condition B: Retention time=1.81 min; ESI-MS(+) m/z [M+H]⁺: 1971.2.

### Preparation of Compound 2079

Compound 2079 was prepared on a 25 μmol scale. The yield of the product was 29.5 mg, and its estimated purity by LCMS analysis was 95.9%. Analysis condition A: Retention time=1.68 min; ESI-MS(+) m/z [M+H]⁺: 1954.

### Preparation of Compound 2080

Compound 2080 was prepared on a 25 μmol scale. The yield of the product was 14.7 mg, and its estimated purity by LCMS analysis was 96.4%. Analysis condition B: Retention time=1.47 min; ESI-MS(+) m/z [M+2H]²⁺: 915.6.

### Preparation of Compound 2081

Compound 2081 was prepared on a 25 μmol scale. The yield of the product was 18 mg, and its estimated purity by LCMS analysis was 96.6%. Analysis condition A: Retention time=1.76 min; ESI-MS(+) m/z [M+H]⁺: 1892.

### Preparation of Compound 2082

Compound 2082 was prepared on a 25 μmol scale. The yield of the product was 29.3 mg, and its estimated purity by LCMS analysis was 100%. Analysis condition A: Retention time=1.62 min; ESI-MS(+) m/z [M+2H]²⁺: 922.9.

### Preparation of Compound 2083

Compound 2083 was prepared on a 25 μmol scale. The yield of the product was 20.4 mg, and its estimated purity by LCMS analysis was 100%. Analysis condition A: Retention time=1.63 min; ESI-MS(+) m/z [M+2H]²⁺: 936.9.

### Preparation of Compound 2084

Compound 2084 was prepared on a 25 μmol scale. The yield of the product was 13 mg, and its estimated purity by LCMS analysis was 100%. Analysis condition A: Retention time=1.59 min; ESI-MS(+) m/z [M+H]⁺: 1887.

### Preparation of Compound 2085

Compound 2085 was prepared on a 50 μmol scale. The yield of the product was 21.5 mg, and its estimated purity by LCMS analysis was 100%. Analysis condition A: Retention time=1.51 min; ESI-MS(+) m/z [M+H]⁺: 1957.1.

### Preparation of Compound 2086

Compound 2086 was prepared on a 50 μmol scale. The yield of the product was 28.7 mg, and its estimated purity by LCMS analysis was 98.8%. Analysis condition A: Retention time=1.37 min; ESI-MS(+) m/z [M+H]⁺: 1917.1.

### Preparation of Compound 2087

Compound 2087 was prepared on a 25 μmol scale. The yield of the product was 14 mg, and its estimated purity by LCMS analysis was 97.3%. Analysis condition B: Retention time=1.55 min; ESI-MS(+) m/z [M+2H]²⁺: 959.

### Preparation of Compound 2088

Compound 2088 was prepared on a 50 μmol scale. The yield of the product was 31.1 mg, and its estimated purity by LCMS analysis was 93.3%. Analysis condition A: Retention time=1.8 min; ESI-MS(+) m/z [M+H]⁺: 1946.2.

### Preparation of Compound 2089

Compound 2089 was prepared on a 50 μmol scale. The yield of the product was 35.2 mg, and its estimated purity by LCMS analysis was 97%. Analysis condition B: Retention time=1.72 min; ESI-MS(+) m/z [M+H]⁺: 1988.

### Preparation of Compound 2090

Compound 2090 was prepared on a 50 μmol scale. The yield of the product was 33.9 mg, and its estimated purity by LCMS analysis was 95.6%. Analysis condition A: Retention time=1.64 min; ESI-MS(+) m/z [M+2H]²⁺: 995.3.

### Preparation of Compound 2091

Compound 2091 was prepared on a 50 μmol scale. The yield of the product was 19.3 mg, and its estimated purity by LCMS analysis was 95.8%. Analysis condition B: Retention time=1.75 min; ESI-MS(+) m/z [M+H]⁺: 1924.7.

### Preparation of Compound 2092

Compound 2092 was prepared on a 50 μmol scale. The yield of the product was 41.2 mg, and its estimated purity by LCMS analysis was 95%. Analysis condition B: Retention time=1.88 min; ESI-MS(+) m/z [M+2H]²⁺: 1023.9.

### Preparation of Compound 2093

Compound 2093 was prepared on a 50 μmol scale. The yield of the product was 18.1 mg, and its estimated purity by LCMS analysis was 95.7%. Analysis condition A: Retention time=1.76 min; ESI-MS(+) m/z [M+H]⁺: 1968.2.

### Preparation of Compound 2094

Compound 2094 was prepared on a 50 μmol scale. The yield of the product was 16.5 mg, and its estimated purity by LCMS analysis was 96.5%. Analysis condition B: Retention time=1.7 min; ESI-MS(+) m/z [M+2H]²⁺: 978.1.

### Preparation of Compound 2095

Compound 2095 was prepared on a 50 μmol scale. The yield of the product was 11.3 mg, and its estimated purity by LCMS analysis was 98.9%. Analysis condition B: Retention time=1.8 min; ESI-MS(+) m/z [M+2H]²⁺: 942.9.

### Preparation of Compound 2096

Compound 2096 was prepared on a 25 μmol scale. The yield of the product was 12.8 mg, and its estimated purity by LCMS analysis was 95.1%. Analysis condition A: Retention time=1.06 min; ESI-MS(+) m/z [M+2H]²⁺: 1008.

### Preparation of Compound 2097

Compound 2097 was prepared on a 25 μmol scale. The yield of the product was 18.8 mg, and its estimated purity by LCMS analysis was 96.5%. Analysis condition B: Retention time=1.53 min; ESI-MS(+) m/z [M+2H]²⁺: 1068.1.

### Preparation of Compound 2098

Compound 2098 was prepared on a 25 μmol scale. The yield of the product was 28 mg, and its estimated purity by LCMS analysis was 97.3%. Analysis condition B: Retention time=1.45 min; ESI-MS(+) m/z [M+2H]²⁺: 1014.

### Preparation of Compound 2099

Compound 2099 was prepared on a 25 μmol scale. The yield of the product was 4.9 mg, and its estimated purity by LCMS analysis was 96.7%. Analysis condition B: Retention time=1.34 min; ESI-MS(+) m/z [M+2H]²⁺: 1021.6.

### Preparation of Compound 2100

Compound 2100 was prepared on a 50 μmol scale. The yield of the product was 51.5 mg, and its estimated purity by LCMS analysis was 98%. Analysis condition B: Retention time=1.44 min; ESI-MS(+) m/z [M+2H]²⁺: 1032.1.

### Preparation of Compound 2101

Compound 2101 was prepared on a 50 μmol scale. The yield of the product was 68.3 mg, and its estimated purity by LCMS analysis was 95.6%. Analysis condition A: Retention time=1.46 min; ESI-MS(+) m/z [M+2H]²⁺: 1044.1.

### Preparation of Compound 2102

Compound 2102 was prepared on a 50 μmol scale. The yield of the product was 25 mg, and its estimated purity by LCMS analysis was 99.2%. Analysis condition B: Retention time=1.8 min; ESI-MS(+) m/z [M+2H]²⁺: 1024.1.

### Preparation of Compound 2103

Compound 2103 was prepared on a 50 μmol scale. The yield of the product was 16.7 mg, and its estimated purity by LCMS analysis was 97.5%. Analysis condition B: Retention time=1.87 min; ESI-MS(+) m/z [M+2H]²⁺: 1038.

### Preparation of Compound 2104

Compound 2104 was prepared on a 50 μmol scale. The yield of the product was 9.9 mg, and its estimated purity by LCMS analysis was 99.3%. Analysis condition A: Retention time=1.57 min; ESI-MS(+) m/z [M+2H]²⁺: 1057.2.

### Preparation of Compound 2105

Compound 2105 was prepared on a 50 μmol scale. The yield of the product was 14.4 mg, and its estimated purity by LCMS analysis was 97.5%. Analysis condition B: Retention time=1.98 min; ESI-MS(+) m/z [M+2H]²⁺: 1046.2.

### Preparation of Compound 2106

Compound 2106 was prepared on a 50 μmol scale. The yield of the product was 33.2 mg, and its estimated purity by LCMS analysis was 99.2%. Analysis condition A: Retention time=1.7 min; ESI-MS(+) m/z [M+2H]²⁺: 1037.1.

### Preparation of Compound 2107

Compound 2107 was prepared on a 50 μmol scale. The yield of the product was 7.3 mg, and its estimated purity by LCMS analysis was 95%. Analysis condition A: Retention time=1.6 min; ESI-MS(+) m/z [M+2H]²⁺: 1065.2.

### Preparation of Compound 2108

Compound 2108 was prepared on a 50 μmol scale. The yield of the product was 103.9 mg, and its estimated purity by LCMS analysis was 95%. Analysis condition A: Retention time=1.72 min; ESI-MS(+) m/z [M+2H]²⁺: 1032.2.

### Preparation of Compound 2109

Compound 2109 was prepared on a 50 μmol scale. The yield of the product was 40.1 mg, and its estimated purity by LCMS analysis was 93.5%. Analysis condition A: Retention time=1.76 min; ESI-MS(+) m/z [M+2H]²⁺: 1046.3.

### Preparation of Compound 2110

Compound 2110 was prepared on a 50 μmol scale. The yield of the product was 35.1 mg, and its estimated purity by LCMS analysis was 99.4%. Analysis condition B: Retention time=1.71 min; ESI-MS(+) m/z [M+2H]²⁺: 1038.1.

### Preparation of Compound 2111

Compound 2111 was prepared on a 50 μmol scale. The yield of the product was 33.5 mg, and its estimated purity by LCMS analysis was 98.5%. Analysis condition B: Retention time=1.78 min; ESI-MS(+) m/z [M+H]⁺: 1969.2.

### Preparation of Compound 2112

Compound 2112 was prepared on a 50 μmol scale. The yield of the product was 52.5 mg, and its estimated purity by LCMS analysis was 100%. Analysis condition A: Retention time=1.56 min; ESI-MS(+) m/z [M+H]⁺: 1995.9.

### Preparation of Compound 2113

Compound 2113 was prepared on a 50 μmol scale. The yield of the product was 22.2 mg, and its estimated purity by LCMS analysis was 97.1%. Analysis condition: Retention time=1.6 min; ESI-MS(+) m/z [M+2H]²⁺: 1051.9.

### Preparation of Compound 2114

Compound 2114 was prepared on a 50 μmol scale. The yield of the product was 30 mg, and its estimated purity by LCMS analysis was 95.5%. Analysis condition: Retention time=1.81 min; ESI-MS(+) m/z [M+H]⁺: 1971.1.

### Preparation of Compound 2115

Compound 2115 was prepared on a 50 μmol scale. The yield of the product was 45.3 mg, and its estimated purity by LCMS analysis was 95.8%. Analysis condition: Retention time=1.78 min; ESI-MS(+) m/z [M+2H]²⁺: 1074.

### Preparation of Compound 2116

Compound 2116 was prepared on a 50 μmol scale. The yield of the product was 44.5 mg, and its estimated purity by LCMS analysis was 99.2%. Analysis condition A: Retention time=1.57 min; ESI-MS(+) m/z [M+H]⁺: 1945.9.

### Preparation of Compound 2117

Compound 2117 was prepared on a 50 μmol scale. The yield of the product was 23.3 mg, and its estimated purity by LCMS analysis was 96.3%. Analysis condition: Retention time=1.7 min; ESI-MS(+) m/z [M+2H]²⁺: 1972.3.

### Preparation of Compound 2118

Compound 2118 was prepared on a 50 μmol scale. The yield of the product was 20.8 mg, and its estimated purity by LCMS analysis was 99.3%. Analysis condition A: Retention time=1.6 min; ESI-MS(+) m/z [M+2H]²⁺: 1041.1.

### Preparation of Compound 2119

Compound 2119 was prepared on a 50 μmol scale. The yield of the product was 52.4 mg, and its estimated purity by LCMS analysis was 98.1%. Analysis condition B: Retention time=1.76 min; ESI-MS(+) m/z [M+2H]²⁺: 1028.2.

### Preparation of Compound 2120

Compound 2120 was prepared on a 50 μmol scale. The yield of the product was 40.4 mg, and its estimated purity by LCMS analysis was 99.1%. Analysis condition B: Retention time=1.85 min; ESI-MS(+) m/z [M+2H]²⁺: 1013.1.

### Preparation of Compound 2121

Compound 2121 was prepared on a 50 μmol scale. The yield of the product was 25.7 mg, and its estimated purity by LCMS analysis was 98.4%. Analysis condition B: Retention time=1.86 min; ESI-MS(+) m/z [M+H]⁺: 1998.1.

### Preparation of Compound 2122

Compound 2122 was prepared on a 50 μmol scale. The yield of the product was 25.8 mg, and its estimated purity by LCMS analysis was 95.5%. Analysis condition A: Retention time=1.39 min; ESI-MS(+) m/z [M+2H]²⁺: 1034.2.

### Preparation of Compound 2123

Compound 2123 was prepared on a 50 μmol scale. The yield of the product was 27 mg, and its estimated purity by LCMS analysis was 97.7%. Analysis condition B: Retention time=1.7 min; ESI-MS(+) m/z [M+2H]²⁺: 1005.1.

### Preparation of Compound 2124

Compound 2124 was prepared on a 50 μmol scale. The yield of the product was 11.5 mg, and its estimated purity by LCMS analysis was 95.9%. Analysis condition A: Retention time=1.65 min; ESI-MS(+) m/z [M+2H]²⁺: 1087.4.

### Preparation of Compound 2125

Compound 2125 was prepared on a 50 μmol scale. The yield of the product was 27.2 mg, and its estimated purity by LCMS analysis was 95.1%. Analysis condition B: Retention time=1.72 min; ESI-MS(+) m/z [M+2H]²⁺: 998.1.

### Preparation of Compound 2126

Compound 2126 was prepared on a 50 μmol scale. The yield of the product was 9.3 mg, and its estimated purity by LCMS analysis was 96.4%. Analysis condition: Retention time=1.76 min; ESI-MS(+) m/z [M+2H]²⁺: 1119.2.

### Preparation of Compound 2127

Compound 2127 was prepared on a 50 μmol scale. The yield of the product was 41 mg, and its estimated purity by LCMS analysis was 95.7%. Analysis condition A: Retention time=1.69 min; ESI-MS(+) m/z [M+2H]²⁺: 1055.

### Preparation of Compound 2128

Compound 2128 was prepared on a 50 μmol scale. The yield of the product was 44.1 mg, and its estimated purity by LCMS analysis was 100%. Analysis condition 3: Retention time=1.58 min; ESI-MS(+) m/z [M+2H]²⁺: 1100.1.

### Preparation of Compound 2129

Compound 2129 was prepared on a 50 μmol scale. The yield of the product was 35.2 mg, and its estimated purity by LCMS analysis was 98.4%. Analysis condition 4: Retention time=1.78 min; ESI-MS(+) m/z [M+2H]²⁺: 1076.1.

### Preparation of Compound 2130

Compound 2130 was prepared on a 50 μmol scale. The yield of the product was 33.6 mg, and its estimated purity by LCMS analysis was 95.7%. Analysis condition: Retention time=1.51 min; ESI-MS(+) m/z [M+2H]²⁺: 1114.9.

### Preparation of Compound 2131

Compound 2131 was prepared on a 50 μmol scale. The yield of the product was 42.6 mg, and its estimated purity by LCMS analysis was 90.9%. Analysis condition: Retention time=1.46 min; ESI-MS(+) m/z [M+2H]²⁺: 1120.3.

### Preparation of Compound 2132

Compound 2132 was prepared on a 50 μmol scale. The yield of the product was 24.6 mg, and its estimated purity by LCMS analysis was 96.4%. Analysis condition: Retention time=1.5 min; ESI-MS(+) m/z [M+2H]²⁺: 1133.2.

### Preparation of Compound 2133

Compound 2133 was prepared on a 50 μmol scale. The yield of the product was 34.1 mg, and its estimated purity by LCMS analysis was 95%. Analysis condition: Retention time=1.53 min; ESI-MS(+) m/z [M+2H]²⁺: 1100.9.

### Preparation of Compound 2134

Compound 2134 was prepared on a 50 μmol scale. The yield of the product was 21.2 mg, and its estimated purity by LCMS analysis was 95.6%. Analysis condition 5: Retention time=1.69 min; ESI-MS(+) m/z [M+2H]²⁺: 1098.2.

### Preparation of Compound 2135

Compound 2135 was prepared on a 50 μmol scale. The yield of the product was 24.9 mg, and its estimated purity by LCMS analysis was 95%. Analysis condition 6: Retention time=1.69 min; ESI-MS(+) m/z [M+2H]²⁺: 1113.3.

### Preparation of Compound 2136

Compound 2136 was prepared on a 30 μmol scale. The yield of the product was 20.8 mg, and its estimated purity by LCMS analysis was 96.9%. Analysis condition: Retention time=1.51, 1.56 min; ESI-MS(+) m/z [M+2H]²⁺: 1093.05, 1093.09.

### Preparation of Compound 2137

Compound 2137 was prepared on a 50 μmol scale. The yield of the product was 59.8 mg, and its estimated purity by LCMS analysis was 94.9%. Analysis condition B: Retention time=1.77 min; ESI-MS(+) m/z [M+2H]²⁺: 712.3.

### Preparation of Compound 2138

Compound 2138 was prepared on a 50 μmol scale. The yield of the product was 1.1 mg, and its estimated purity by LCMS analysis was 100%. Analysis condition A: Retention time=1.57 min; ESI-MS(+) m/z [M+2H]²⁺: 1098.2.

### Preparation of Compound 2139

Compound 2139 was prepared on a 40 μmol scale. The yield of the product was 49.6 mg, and its estimated purity by LCMS analysis was 98.9%. Analysis condition: Retention time=1.75 min; ESI-MS(+) m/z [M+2H]²⁺: 1018.1.

### Preparation of Compound 2140

Compound 2140 was prepared on a 40 μmol scale. The yield of the product was 40 mg, and its estimated purity by LCMS analysis was 96.3%. Analysis condition B: Retention time=1.75 min; ESI-MS(+) m/z [M+2H]²⁺: 1062.4.

### Preparation of Compound 2141

Compound 2141 was prepared on a 40 μmol scale. The yield of the product was 28.5 mg, and its estimated purity by LCMS analysis was 98.6%. Analysis condition: Retention time=1.71 min; ESI-MS(+) m/z [M+H]⁺: 1986.3.

### Preparation of Compound 2142

Compound 2142 was prepared on a 40 μmol scale. The yield of the product was 29.1 mg, and its estimated purity by LCMS analysis was 94.9%. Analysis condition B: Retention time=1.8 min; ESI-MS(+) m/z [M+2H]²⁺: 1039.2.

### Preparation of Compound 2143

Compound 2143 was prepared on a 30 μmol scale. The yield of the product was 6.3 mg, and its estimated purity by LCMS analysis was 96.3%. Analysis condition: Retention time=1.79 min; ESI-MS(+) m/z [M+2H]²⁺: 1007.2.

### Preparation of Compound 2144

Compound 2144 was prepared on a 30 μmol scale. The yield of the product was 17.6 mg, and its estimated purity by LCMS analysis was 96.2%. Analysis condition A: Retention time=1.44, 1.48 min; ESI-MS(+) m/z [M+2H]²⁺: 1108.1.

### Preparation of Compound 2145

Compound 2145 was prepared on a 30 μmol scale. The yield of the product was 21.6 mg, and its estimated purity by LCMS analysis was 95.1%. Analysis condition: Retention time=1.8 min; ESI-MS(+) m/z [M+3H]³⁺: 675.3.

### Preparation of Compound 2146

Compound 2146 was prepared on a 30 μmol scale. The yield of the product was 19.4 mg, and its estimated purity by LCMS analysis was 97.3%. Analysis condition A: Retention time=1.54 min; ESI-MS(+) m/z [M+2H]²⁺: 1105.

### Preparation of Compound 2147

Compound 2147 was prepared on a 30 μmol scale. The yield of the product was 27.4 mg, and its estimated purity by LCMS analysis was 95.7%. Analysis condition A: Retention time=1.71 min: ESI-MS(+) m/z [M+H]⁺: 1976.1.

### Preparation of Compound 2148

Compound 2148 was prepared on a 30 μmol scale. The yield of the product was 46 mg, and its estimated purity by LCMS analysis was 95%. Analysis condition B: Retention time=1.74 min; ESI-MS(+) m/z [M+2H]²⁺: 1082.1.

### Preparation of Compound 2149

Compound 2149 was prepared on a 50 μmol scale. The yield of the product was 15.8 mg, and its estimated purity by LCMS analysis was 97.8%. Analysis condition 7: Retention time=1.78 min; ESI-MS(+) m/z [M+2H]²⁺: 1089.9.

### Preparation of Compound 2150

Compound 2150 was prepared on a 50 μmol scale. The yield of the product was 12.4 mg, and its estimated purity by LCMS analysis was 95.1%. Analysis condition 8: Retention time=1.78 min; ESI-MS(+) m/z [M+2H]²⁺: 1074.9.

### Preparation of Compound 2151

Compound 2151 was prepared on a 50 μmol scale. The yield of the product was 6.5 mg, and its estimated purity by LCMS analysis was 97.8%. Analysis condition B: Retention time=1.78 min; ESI-MS(+) m/z [M+2H]²⁺: 1075.

### Preparation of Compound 2152

Compound 2152 was prepared on a 50 μmol scale. The yield of the product was 19.8 mg, and its estimated purity by LCMS analysis was 99%. Analysis condition: Retention time=1.86 min; ESI-MS(+) m/z [M+2H]²⁺: 1059.3.

### Preparation of Compound 2153

Compound 2153 was prepared on a 50 μmol scale. The yield of the product was 13.8 mg, and its estimated purity by LCMS analysis was 99.2%. Analysis condition B: Retention time=1.71 min; ESI-MS(+) m/z [M+H]⁺: 1985.3.

### Preparation of Compound 2154

Compound 2154 was prepared on a 50 μmol scale. The yield of the product was 20.4 mg, and its estimated purity by LCMS analysis was 95.5%. Analysis condition B: Retention time=1.66 min; ESI-MS(+) m/z [M+3H]³⁺: 726.2.

### Preparation of Compound 2155

Compound 2155 was prepared on a 50 μmol scale. The yield of the product was 58.2 mg, and its estimated purity by LCMS analysis was 95%. Analysis condition B: Retention time=1.67 min; ESI-MS(+) m/z [M+2H]²⁺: 1083.2.

### Preparation of Compound 2156

Compound 2156 was prepared on a 50 μmol scale. The yield of the product was 116.3 mg, and its estimated purity by LCMS analysis was 95.4%. Analysis condition: Retention time=1.54 min; ESI-MS(+) m/z [M+2H]²⁺: 1081.

### Preparation of Compound 2157

Compound 2157 was prepared on a 50 μmol scale. The yield of the product was 35.9 mg, and its estimated purity by LCMS analysis was 95.7%. Analysis condition A: Retention time=1.9 min; ESI-MS(+) m/z [M+H]⁺: 1959.1.

### Preparation of Compound 2158

Compound 2158 was prepared on a 30 μmol scale. The yield of the product was 2.1 mg, and its estimated purity by LCMS analysis was 95.5%. Analysis condition A: Retention time=1.59 min; ESI-MS(+) m/z [M+3H]³⁺: 733.4.

### Preparation of Compound 2159

Compound 2159 was prepared on a 30 μmol scale. The yield of the product was 21 mg, and its estimated purity by LCMS analysis was 96.2%. Analysis condition B: Retention time=1.64 min; ESI-MS(+) m/z [M+2H]²⁺: 1123.1.

### Preparation of Compound 2160

Compound 2160 was prepared on a 30 μmol scale. The yield of the product was 8.3 mg, and its estimated purity by LCMS analysis was 97.1%. Analysis condition B: Retention time=1.78 min; ESI-MS(+) m/z [M+2H]²⁺: 1125.

### Preparation of Compound 2161

Compound 2161 was prepared on a 30 μmol scale. The yield of the product was 16.9 mg, and its estimated purity by LCMS analysis was 97.4%. Analysis condition 9: Retention time=1.49 min; ESI-MS(+) m/z [M+2H]²⁺: 1121.2.

### Preparation of Compound 2162

Compound 2162 was prepared on a 30 μmol scale. The yield of the product was 19.8 mg, and its estimated purity by LCMS analysis was 95.8%. Analysis condition B0: Retention time=1.66 min; ESI-MS(+) m/z [M+3H]³⁺: 763.2.

### Preparation of Compound 2163

Compound 2163 was prepared on a 30 μmol scale. The yield of the product was 29.6 mg, and its estimated purity by LCMS analysis was 96%. Analysis condition B1: Retention time=1.8 min; ESI-MS(+) m/z [M+2H]²⁺: 1147.1.

### Preparation of Compound 2164

Compound 2164 was prepared on a 30 μmol scale. The yield of the product was 33.6 mg, and its estimated purity by LCMS analysis was 94.4%. Analysis condition B2: Retention time=1.54 min; ESI-MS(+) m/z [M+2H]²⁺: 1142.1.

### Preparation of Compound 2165

Compound 2165 was prepared on a 30 μmol scale. The yield of the product was 7.8 mg, and its estimated purity by LCMS analysis was 96.6%. Analysis condition B3: Retention time=1.71 min; ESI-MS(+) m/z [M+3H]³⁺: 777.

### Preparation of Compound 2166

Compound 2166 was prepared on a 30 μmol scale. The yield of the product was 34.8 mg, and its estimated purity by LCMS analysis was 97.9%. Analysis condition B4: Retention time=1.55 min; ESI-MS(+) m/z [M+2H]²⁺: 1168.1.

### Preparation of Compound 2167

Compound 2167 was prepared on a 30 μmol scale. The yield of the product was 19.9 mg, and its estimated purity by LCMS analysis was 100%. Analysis condition B5: Retention time=1.61 min; ESI-MS(+) m/z [M+2H]²⁺: 1058.2.

### Preparation of Compound 2168

Compound 2168 was prepared on a 30 μmol scale. The yield of the product was 14.4 mg, and its estimated purity by LCMS analysis was 99.3%. Analysis condition B: Retention time=1.72 min; ESI-MS(+) m/z [M+3H]³⁺: 721.2.

### Preparation of Compound 2169

Compound 2169 was prepared on a 30 μmol scale. The yield of the product was 14.1 mg, and its estimated purity by LCMS analysis was 99.3%. Analysis condition B6: Retention time=1.85 min; ESI-MS(+) m/z [M+2H]²⁺: 1084.1.

### Preparation of Compound 2170

Compound 2170 was prepared on a 50 μmol scale. The yield of the product was 21.5 mg, and its estimated purity by LCMS analysis was 99.2%. Analysis condition A: Retention time=1.65 min; ESI-MS(+) m/z [M+H]⁺: 1960.7.

### Preparation of Compound 2171

Compound 2171 was prepared on a 30 μmol scale. The yield of the product was 9.1 mg, and its estimated purity by LCMS analysis was 99.1%. Analysis condition B: Retention time=1.96 min; ESI-MS(+) m/z [M+H]⁺: 1997.

### Preparation of Compound 2172

Compound 2172 was prepared on a 30 μmol scale. The yield of the product was 15.1 mg, and its estimated purity by LCMS analysis was 95.2%. Analysis condition B7: Retention time=1.88 min; ESI-MS(+) m/z [M+2H]²⁺: 1026.2.

### Preparation of Compound 2173

Compound 2173 was prepared on a 30 μmol scale. The yield of the product was 21.3 mg, and its estimated purity by LCMS analysis was 96.4%. Analysis condition A: Retention time=1.97 min; ESI-MS(+) m/z [M+H]⁺: 1941.1.

### Preparation of Compound 2174

Compound 2174 was prepared on a 30 μmol scale. The yield of the product was 20.3 mg, and its estimated purity by LCMS analysis was 100%. Analysis condition A: Retention time=1.9 min; ESI-MS(+) m/z [M+H]⁺: 1992.2.

### Preparation of Compound 2175

Compound 2175 was prepared on a 30 μmol scale. The yield of the product was 13.5 mg, and its estimated purity by LCMS analysis was 90.2%. Analysis condition B: Retention time=2.02 min; ESI-MS(+) m/z [M+H]⁺: 1983.1.

### Preparation of Compound 2176

Compound 2176 was prepared on a 30 μmol scale. The yield of the product was 4.4 mg, and its estimated purity by LCMS analysis was 88.4%. Analysis condition B: Retention time=2.11 min; ESI-MS(+) m/z [M+2H]²⁺: 1010.2.

### Preparation of Compound 2177

Compound 2177 was prepared on a 30 μmol scale. The yield of the product was 35.9 mg, and its estimated purity by LCMS analysis was 100%. Analysis condition A: Retention time=1.52 min; ESI-MS(+) m/z [M+2H]²⁺: 1071.

### Preparation of Compound 2178

Compound 2178 was prepared on a 30 μmol scale. The yield of the product was 23.8 mg, and its estimated purity by LCMS analysis was 100%. Analysis condition A: Retention time=1.52 min; ESI-MS(+) m/z [M+2H]²⁺: 1051.

### Preparation of Compound 2179

Compound 2179 was prepared on a 30 μmol scale. The yield of the product was 20.1 mg, and its estimated purity by LCMS analysis was 97.2%. Analysis condition A: Retention time=1.58 min; ESI-MS(+) m/z [M+2H]²⁺: 1052.3.

### Preparation of Compound 2180

Compound 2180 was prepared on a 30 μmol scale. The yield of the product was 21.8 mg, and its estimated purity by LCMS analysis was 95.5%. Analysis condition B: Retention time=1.75 min; ESI-MS(+) m/z [M+2H]²⁺: 1076.2.

### Preparation of Compound 2181

Compound 2181 was prepared on a 30 μmol scale. The yield of the product was 18.4 mg, and its estimated purity by LCMS analysis was 95.4%. Analysis condition B: Retention time=1.82 min; ESI-MS(+) m/z [M+2H]²⁺: 1053.

### Preparation of Compound 2182

Compound 2182 was prepared on a 50 μmol scale. The yield of the product was 41.6 mg, and its estimated purity by LCMS analysis was 99.1%. Analysis condition A: Retention time=1.51 min; ESI-MS(+) m/z [M+2H]²⁺: 1078.1.

### Preparation of Compound 2183

Compound 2183 was prepared on a 50 μmol scale. The yield of the product was 68.5 mg, and its estimated purity by LCMS analysis was 100%. Analysis condition A: Retention time=1.44 min; ESI-MS(+) m/z [M+2H]²⁺: 1084.2.

### Preparation of Compound 2184

Compound 2184 was prepared on a 50 μmol scale. The yield of the product was 55.2 mg, and its estimated purity by LCMS analysis was 95.3%. Analysis condition B: Retention time=1.8 min; ESI-MS(+) m/z [M+2H]²⁺: 1084.1.

### Preparation of Compound 2185

Compound 2185 was prepared on a 50 μmol scale. The yield of the product was 40.5 mg, and its estimated purity by LCMS analysis was 92.5%. Analysis condition B: Retention time=1.95 min; ESI-MS(+) m/z [M+2H]²⁺: 1086.1.

### Preparation of Compound 2186

Compound 2186 was prepared on a 50 μmol scale. The yield of the product was 61.9 mg, and its estimated purity by LCMS analysis was 100%. Analysis condition A: Retention time=1.7 min; ESI-MS(+) m/z [M+H]⁺: 1994.1.

### Preparation of Compound 2187

Compound 2187 was prepared on a 50 μmol scale. The yield of the product was 17.6 mg, and its estimated purity by LCMS analysis was 100%. Analysis condition B: Retention time=1.69 min; ESI-MS(+) m/z [M+3H]³⁺: 751.

### Preparation of Compound 2188

Compound 2188 was prepared on a 50 μmol scale. The yield of the product was 48.7 mg, and its estimated purity by LCMS analysis was 91.1%. Analysis condition A: Retention time=1.42 min; ESI-MS(+) m/z [M+2H]²⁺: 1134.1.

### Preparation of Compound 2189

Compound 2189 was prepared on a 50 μmol scale. The yield of the product was 28.2 mg, and its estimated purity by LCMS analysis was 97%. Analysis condition A: Retention time=1.35, 1.39 min; ESI-MS(+) m/z [M+2H]²⁺: 1119.2.

### Preparation of Compound 2190

Compound 2190 was prepared on a 50 μmol scale. The yield of the product was 32.7 mg, and its estimated purity by LCMS analysis was 99.4%. Analysis condition B: Retention time=1.75 min; ESI-MS(+) m/z [M+2H]²⁺: 1127.2.

### Preparation of Compound 2191

Compound 2191 was prepared on a 50 μmol scale. The yield of the product was 50.3 mg, and its estimated purity by LCMS analysis was 95.5%. Analysis condition A: Retention time=1.48 min; ESI-MS(+) m/z [M+2H]²⁺: 1127.

### Preparation of Compound 2192

Compound 2192 was prepared on a 50 μmol scale. The yield of the product was 43.5 mg, and its estimated purity by LCMS analysis was 98.7%. Analysis condition B: Retention time=1.7 min; ESI-MS(+) m/z [M+3H]³⁺: 756.2.

### Preparation of Compound 2193

Compound 2193 was prepared on a 50 μmol scale. The yield of the product was 8.8 mg, and its estimated purity by LCMS analysis was 96.2%. Analysis condition B: Retention time=1.73 min; ESI-MS(+) m/z [M+2H]²⁺: 1111.3.

### Preparation of Compound 2194

Compound 2194 was prepared on a 50 μmol scale. The yield of the product was 27.1 mg, and its estimated purity by LCMS analysis was 99.1%. Analysis condition B: Retention time=1.78 min; ESI-MS(+) m/z [M+2H]²⁺: 1112.9.

### Preparation of Compound 2195

Compound 2195 was prepared on a 50 μmol scale. The yield of the product was 41.9 mg, and its estimated purity by LCMS analysis was 96.4%. Analysis condition A: Retention time=1.32 min; ESI-MS(+) m/z [M+2H]²⁺: 1098.

### Preparation of Compound 2196

Compound 2196 was prepared on a 50 μmol scale. The yield of the product was 29.7 mg, and its estimated purity by LCMS analysis was 98.3%. Analysis condition B: Retention time=1.73 min; ESI-MS(+) m/z [M+3H]³⁺: 736.2.

### Preparation of Compound 2197

Compound 2197 was prepared on a 50 μmol scale. The yield of the product was 77 mg, and its estimated purity by LCMS analysis was 100%. Analysis condition B: Retention time=1.77 min; ESI-MS(+) m/z [M+2H]²⁺: 1129.9.

### Preparation of Compound 2198

Compound 2198 was prepared on a 50 μmol scale. The yield of the product was 48.9 mg, and its estimated purity by LCMS analysis was 95.4%. Analysis condition A: Retention time=1.38 min; ESI-MS(+) m/z [M+2H]²⁺: 1115.2.

### Preparation of Compound 2199

Compound 2199 was prepared on a 50 μmol scale. The yield of the product was 60 mg, and its estimated purity by LCMS analysis was 89%. Analysis condition A: Retention time=1.68 min; ESI-MS(+) m/z [M+2H]²⁺: 1029.2.

### Preparation of Compound 2200

Compound 2200 was prepared on a 50 μmol scale. The yield of the product was 69 mg, and its estimated purity by LCMS analysis was 95%. Analysis condition A: Retention time=1.5 min; ESI-MS(+) m/z [M+2H]²⁺: 1143.1.

### Preparation of Compound 2201

Compound 2201 was prepared on a 50 μmol scale. The yield of the product was 45.4 mg, and its estimated purity by LCMS analysis was 96.2%. Analysis condition A: Retention time=1.58, 1.62 min; ESI-MS(+) m/z [M+2H]²⁺: 1081.

### Preparation of Compound 2202

Compound 2202 was prepared on a 50 μmol scale. The yield of the product was 28.2 mg, and its estimated purity by LCMS analysis was 90.8%. Analysis condition A: Retention time=1.37 min; ESI-MS(+) m/z [M+2H]²⁺: 1082.2.

### Preparation of Compound 2203

Compound 2203 was prepared on a 50 μmol scale. The yield of the product was 4.4 mg, and its estimated purity by LCMS analysis was 94.4%. Analysis condition B: Retention time=1.61 min; ESI-MS(+) m/z [M+2H]²⁺: 1100.1.

### Preparation of Compound 2204

Compound 2204 was prepared on a 50 μmol scale. The yield of the product was 41.6 mg, and its estimated purity by LCMS analysis was 95.2%. Analysis condition A: Retention time=1.65 min; ESI-MS(+) m/z [M+2H]²⁺: 1005.4.

### Preparation of Compound 2205

Compound 2205 was prepared on a 50 μmol scale. The yield of the product was 11.8 mg, and its estimated purity by LCMS analysis was 100%. Analysis condition A: Retention time=1.64 min; ESI-MS(+) m/z [M+2H]²¹: 1058.1.

### Preparation of Compound 2206

Compound 2206 was prepared on a 50 μmol scale. The yield of the product was 31 mg, and its estimated purity by LCMS analysis was 96%. Analysis condition A: Retention time=1.8 min; ESI-MS(+) m/z [M+H]⁺: 1956.3.

### Preparation of Compound 2207

Compound 2207 was prepared on a 50 μmol scale. The yield of the product was 78 mg, and its estimated purity by LCMS analysis was 95.3%. Analysis condition B: Retention time=1.89 min; ESI-MS(+) m/z [M+2H]²⁺: 1098.2.

### Preparation of Compound 2208

Compound 2208 was prepared on a 50 μmol scale. The yield of the product was 58.5 mg, and its estimated purity by LCMS analysis was 91%. Analysis condition A: Retention time=1.56 min; ESI-MS(+) m/z [M+2H]²⁺: 1090.

### Preparation of Compound 2209

Compound 2209 was prepared on a 50 μmol scale. The yield of the product was 53.5 mg, and its estimated purity by LCMS analysis was 96.7%. Analysis condition A: Retention time=1.42 min; ESI-MS(+) m/z [M+2H]²⁺: 1091.

### Preparation of Compound 2210

Compound 2210 was prepared on a 50 μmol scale. The yield of the product was 37.1 mg, and its estimated purity by LCMS analysis was 99.4%. Analysis condition B: Retention time=1.81 min; ESI-MS(+) m/z [M+3H]³⁺: 654.8.

### Preparation of Compound 2211

Compound 2211 was prepared on a 50 μmol scale. The yield of the product was 24.6 mg, and its estimated purity by LCMS analysis was 100%. Analysis condition A: Retention time=1.67 min; ESI-MS(+) m/z [M+2H]²⁺: 1906.7.

### Preparation of Compound 2212

Compound 2212 was prepared on a 50 μmol scale. The yield of the product was 35.8 mg, and its estimated purity by LCMS analysis was 93%. Analysis condition B: Retention time=1.83 min; ESI-MS(+) m/z [M+H]⁺: 1962.2.

### Preparation of Compound 2213

Compound 2213 was prepared on a 50 μmol scale. The yield of the product was 37.1 mg, and its estimated purity by LCMS analysis was 88.6%. Analysis condition B: Retention time=1.91 min; ESI-MS(+) m/z [M+2H]²⁺: 1975.

### Preparation of Compound 2214

Compound 2214 was prepared on a 50 μmol scale. The yield of the product was 38 mg, and its estimated purity by LCMS analysis was 93.6%. Analysis condition B: Retention time=1.8 min; ESI-MS(+) m/z [M+H]⁺: 1989.4.

### Preparation of Compound 2215

Compound 2215 was prepared on a 50 μmol scale. The yield of the product was 4.4 mg, and its estimated purity by LCMS analysis was 97.6%. Analysis condition B: Retention time=1.8 min; ESI-MS(+) m/z [M+2H]²⁺: 1002.9.

### Preparation of Compound 2216

Compound 2216 was prepared on a 50 μmol scale. The yield of the product was 37.5 mg, and its estimated purity by LCMS analysis was 99%. Analysis condition A: Retention time=1.65 min; ESI-MS(+) m/z [M+2H]²⁺: 957.7.

### Preparation of Compound 2217

Compound 2217 was prepared on a 50 μmol scale. The yield of the product was 13.3 mg, and its estimated purity by LCMS analysis was 95.6%. Analysis condition A: Retention time=1.8 min; ESI-MS(+) m/z [M+H]⁺: 1940.9.

### Preparation of Compound 2218

Compound 2218 was prepared on a 50 μmol scale. The yield of the product was 23.8 mg, and its estimated purity by LCMS analysis was 96.3%. Analysis condition B: Retention time=1.76 min; ESI-MS(+) m/z [M+H]⁺: 1866.1.

### Preparation of Compound 2219

Compound 2219 was prepared on a 50 μmol scale. The yield of the product was 45 mg, and its estimated purity by LCMS analysis was 98.8%. Analysis condition A: Retention time=1.87 min; ESI-MS(+) m/z [M+H]⁺: 1967.8.

### Preparation of Compound 2220

Compound 2220 was prepared on a 50 μmol scale. The yield of the product was 16.4 mg, and its estimated purity by LCMS analysis was 100%. Analysis condition A: Retention time=1.58 min; ESI-MS(+) m/z [M+H]⁺: 1947.

### Preparation of Compound 2221

Compound 2221 was prepared on a 50 μmol scale. The yield of the product was 30.4 mg, and its estimated purity by LCMS analysis was 90.9%. Analysis condition A: Retention time=1.44 min; ESI-MS(+) m/z [M+2H]²⁺: 974.

### Preparation of Compound 2222

Compound 2222 was prepared on a 50 μmol scale. The yield of the product was 12.2 mg, and its estimated purity by LCMS analysis was 95.1%. Analysis condition B: Retention time=1.75, 1.79 min; ESI-MS(+) m/z [M+2H]²⁺: 998.

### Preparation of Compound 2223

Compound 2223 was prepared on a 50 μmol scale. The yield of the product was 13.5 mg, and its estimated purity by LCMS analysis was 100%. Analysis condition A: Retention time=1.51 min; ESI-MS(+) m/z [M+2H]²⁺: 994.

### Preparation of Compound 2224

Compound 2224 was prepared on a 50 μmol scale. The yield of the product was 23.5 mg, and its estimated purity by LCMS analysis was 97.1%. Analysis condition A: Retention time=1.71 min; ESI-MS(+) m/z [M+H]⁺: 1971.9.

### Preparation of Compound 2225

Compound 2225 was prepared on a 50 μmol scale. The yield of the product was 40.7 mg, and its estimated purity by LCMS analysis was 85.9%. Analysis condition A: Retention time=1.57 min; ESI-MS(+) m/z [M+H]⁺: 1976.1.

### Preparation of Compound 2226

Compound 2226 was prepared on a 50 μmol scale. The yield of the product was 27.1 mg, and its estimated purity by LCMS analysis was 96.6%. Analysis condition B: Retention time=1.75 min; ESI-MS(+) m/z [M+H]⁺: 1956.8.

### Preparation of Compound 2227

Compound 2227 was prepared on a 50 μmol scale. The yield of the product was 24.7 mg, and its estimated purity by LCMS analysis was 96%. Analysis condition B: Retention time=1.8 min; ESI-MS(+) m/z [M+H]⁺: 1986.2.

### Preparation of Compound 2228

Compound 2228 was prepared on a 50 μmol scale. The yield of the product was 51.9 mg, and its estimated purity by LCMS analysis was 90.2%. Analysis condition A: Retention time=1.66 min; ESI-MS(+) m/z [M+H]⁺: 1946.8.

### Preparation of Compound 2229

Compound 2229 was prepared on a 50 μmol scale. The yield of the product was 10.5 mg, and its estimated purity by LCMS analysis was 99.2%. Analysis condition B: Retention time=1.82 min; ESI-MS(+) m/z [M+2H]²⁺: 995.2.

### Preparation of Compound 2230

Compound 2230 was prepared on a 50 μmol scale. The yield of the product was 43.3 mg, and its estimated purity by LCMS analysis was 98%. Analysis condition B: Retention time=1.79 min; ESI-MS(+) m/z [M+H]⁺: 1960.3.

### Preparation of Compound 2231

Compound 2231 was prepared on a 50 μmol scale. The yield of the product was 36.4 mg, and its estimated purity by LCMS analysis was 95.6%. Analysis condition B: Retention time=1.9 min; ESI-MS(+) m/z [M+2H]²⁺: 1068.2.

### Preparation of Compound 2232

Compound 2232 was prepared on a 50 μmol scale. The yield of the product was 30.5 mg, and its estimated purity by LCMS analysis was 99.1%. Analysis condition B: Retention time=1.84 min; ESI-MS(+) m/z [M+H]⁺: 1935.2.

### Preparation of Compound 2233

Compound 2233 was prepared on a 50 μmol scale. The yield of the product was 5.8 mg, and its estimated purity by LCMS analysis was 91.6%. Analysis condition B: Retention time=1.89 min; ESI-MS(+) m/z [M+2H]²⁺: 954.2.

### Preparation of Compound 2234

Compound 2234 was prepared on a 50 μmol scale. The yield of the product was 4.7 mg, and its estimated purity by LCMS analysis was 95.6%. Analysis condition A: Retention time=1.62 min; ESI-MS(+) m/z [M+H]⁺: 1967.3.

### Preparation of Compound 2235

Compound 2235 was prepared on a 50 μmol scale. The yield of the product was 14.4 mg, and its estimated purity by LCMS analysis was 95.3%. Analysis condition B: Retention time=1.91 min; ESI-MS(+) m/z [M+2H]²⁺: 1906.9.

### Preparation of Compound 2236

Compound 2236 was prepared on a 50 μmol scale. The yield of the product was 8.5 mg, and its estimated purity by LCMS analysis was 96.7%. Analysis condition A: Retention time=1.63 min; ESI-MS(+) m/z [M+2H]²⁺: 1968.

### Preparation of Compound 2237

Compound 2237 was prepared on a 50 μmol scale. The yield of the product was 42.6 mg, and its estimated purity by LCMS analysis was 95.9%. Analysis condition B: Retention time=1.7 min; ESI-MS(+) m/z [M+2H]²⁺: 1024.7.

### Preparation of Compound 2238

Compound 2238 was prepared on a 50 μmol scale. The yield of the product was 23.8 mg, and its estimated purity by LCMS analysis was 94.5%. Analysis condition B: Retention time=2.65 min; ESI-MS(+) m/z [M+3H]³⁺: 669.1.

### Preparation of Compound 2239

Compound 2239 was prepared on a 50 μmol scale. The yield of the product was 31 mg, and its estimated purity by LCMS analysis was 100%. Analysis condition B: Retention time=1.79 min; ESI-MS(+) m/z [M+2H]²⁺: 1038.2.

### Preparation of Compound 2240

Compound 2240 was prepared on a 50 μmol scale. The yield of the product was 32.5 mg, and its estimated purity by LCMS analysis was 96.4%. Analysis condition A: Retention time=1.74 min; ESI-MS(+) m/z [M+2H]²⁺: 1029.9.

### Preparation of Compound 2241

Compound 2241 was prepared on a 25 μmol scale. The yield of the product was 1 mg, and its estimated purity by LCMS analysis was 100%. Analysis condition A: Retention time=1.44 min; ESI-MS(+) m/z [M+2H]²⁺: 1040.9.

### Preparation of Compound 2242

Compound 2242 was prepared on a 50 μmol scale. The yield of the product was 27.3 mg, and its estimated purity by LCMS analysis was 100%. Analysis condition A: Retention time=1.84 min; ESI-MS(+) m/z [M+H]⁺: 1858.5.

### Preparation of Compound 2243

Compound 2243 was prepared on a 50 μmol scale. The yield of the product was 12.4 mg, and its estimated purity by LCMS analysis was 99.2%. Analysis condition B: Retention time=1.88 min; ESI-MS(+) m/z [M+H]⁺: 1932.2.

### Preparation of Compound 2244

Compound 2244 was prepared on a 50 μmol scale. The yield of the product was 30.9 mg, and its estimated purity by LCMS analysis was 99.1%. Analysis condition A: Retention time=1.7 min; ESI-MS(+) m/z [M+2H]²⁺: 967.2.

### Preparation of Compound 2245

Compound 2245 was prepared on a 50 μmol scale. The yield of the product was 24.5 mg, and its estimated purity by LCMS analysis was 99.3%. Analysis condition B: Retention time=1.64 min; ESI-MS(+) m/z [M+H]⁺: 1919.

### Preparation of Compound 2246

Compound 2246 was prepared on a 50 μmol scale. The yield of the product was 29.2 mg, and its estimated purity by LCMS analysis was 97.9%. Analysis condition B: Retention time=1.78 min; ESI-MS(+) m/z [M+H]⁺: 1944.8.

### Preparation of Compound 2247

Compound 2247 was prepared on a 30 μmol scale. The yield of the product was 5.9 mg, and its estimated purity by LCMS analysis was 95.2%. Analysis condition B: Retention time=2.06 min; ESI-MS(+) m/z [M+H]⁺: 1951.9.

### Preparation of Compound 2248

Compound 2248 was prepared on a 50 μmol scale. The yield of the product was 24.8 mg, and its estimated purity by LCMS analysis was 100%. Analysis condition A: Retention time=1.76 min; ESI-MS(+) m/z [M+H]⁺: 1871.3.

### Preparation of Compound 2249

Compound 2249 was prepared on a 50 μmol scale. The yield of the product was 49.7 mg, and its estimated purity by LCMS analysis was 100%. Analysis condition A: Retention time=1.5 min; ESI-MS(+) m/z [M+2H]²⁺: 1024.

### Preparation of Compound 2250

Compound 2250 was prepared on a 50 μmol scale. The yield of the product was 34.8 mg, and its estimated purity by LCMS analysis was 96.2%. Analysis condition B: Retention time=1.95 min; ESI-MS(+) m/z [M+H]⁺: 1846.

### Preparation of Compound 2251

Compound 2251 was prepared on a 50 μmol scale. The yield of the product was 30.6 mg, and its estimated purity by LCMS analysis was 99.2%. Analysis condition B: Retention time=1.8 min; ESI-MS(+) m/z [M+H]⁺: 1857.9.

### Preparation of Compound 2252

Compound 2252 was prepared on a 50 μmol scale. The yield of the product was 18.9 mg, and its estimated purity by LCMS analysis was 94.7%. Analysis condition A: Retention time=1.76 min; ESI-MS(+) m/z [M+H]⁺: 1983.8.

### Preparation of Compound 2253

Compound 2253 was prepared on a 50 μmol scale. The yield of the product was 16.6 mg, and its estimated purity by LCMS analysis was 96.4%. Analysis condition A: Retention time=1.81 min; ESI-MS(+) m/z [M+H]⁺: 1894.2.

### Preparation of Compound 2254

Compound 2254 was prepared on a 50 μmol scale. The yield of the product was 41.7 mg, and its estimated purity by LCMS analysis was 96.2%. Analysis condition B: Retention time=1.76 min; ESI-MS(+) m/z [M+H]⁺: 1993.2.

### Preparation of Compound 2255

Compound 2255 was prepared on a 50 μmol scale. The yield of the product was 34.1 mg, and its estimated purity by LCMS analysis was 97%. Analysis condition B: Retention time=1.75 min; ESI-MS(+) m/z [M+2H]²⁺: 984.1.

### Preparation of Compound 2256

Compound 2256 was prepared on a 50 μmol scale. The yield of the product was 34.5 mg, and its estimated purity by LCMS analysis was 100%. Analysis condition A: Retention time=1.45 min; ESI-MS(+) m/z [M+H]⁺: 1996.1.

### Preparation of Compound 2257

Compound 2257 was prepared on a 50 μmol scale. The yield of the product was 53.9 mg, and its estimated purity by LCMS analysis was 96.3%. Analysis condition A: Retention time=1.52, 1.55 min; ESI-MS(+) m/z [M+H]⁺: 1938.28, 1937.18.

### Preparation of Compound 2258

Compound 2258 was prepared on a 50 μmol scale. The yield of the product was 25.3 mg, and its estimated purity by LCMS analysis was 97.1%. Analysis condition B: Retention time=1.83 min; ESI-MS(+) m/z [M+H]⁺: 1962.3.

### Preparation of Compound 2259

Compound 2259 was prepared on a 50 μmol scale. The yield of the product was 27 mg, and its estimated purity by LCMS analysis was 98.8%. Analysis condition B: Retention time=1.87 min; ESI-MS(+) m/z [M+H]⁺: 1847.

### Preparation of Compound 2260

Compound 2260 was prepared on a 50 μmol scale. The yield of the product was 47.2 mg, and its estimated purity by LCMS analysis was 97.2%. Analysis condition A: Retention time=1.51 min; ESI-MS(+) m/z [M+H]⁺: 1965.1.

### Preparation of Compound 2261

Compound 2261 was prepared on a 50 μmol scale. The yield of the product was 50.7 mg, and its estimated purity by LCMS analysis was 98.3%. Analysis condition A: Retention time=1.61 min; ESI-MS(+) m/z [M+2H]²⁺: 1052.1.

### Preparation of Compound 2262

Compound 2262 was prepared on a 50 μmol scale. The yield of the product was 56.4 mg, and its estimated purity by LCMS analysis was 95.9%. Analysis condition B: Retention time=1.7, 1.74 min; ESI-MS(+) m/z [M+2H]²⁺: 1030.16, 1030.16.

### Preparation of Compound 2263

Compound 2263 was prepared on a 50 μmol scale. The yield of the product was 26.3 mg, and its estimated purity by LCMS analysis was 97.8%. Analysis condition B: Retention time=1.89 min; ESI-MS(+) m/z [M+2H]²⁺: 1019.1.

### Preparation of Compound 2264

Compound 2264 was prepared on a 50 μmol scale. The yield of the product was 25.2 mg, and its estimated purity by LCMS analysis was 95.3%. Analysis condition B: Retention time=1.85 min; ESI-MS(+) m/z [M+2H]²⁺: 1025.

### Preparation of Compound 2265

Compound 2265 was prepared on a 50 μmol scale. The yield of the product was 29.1 mg, and its estimated purity by LCMS analysis was 95.6%. Analysis condition B: Retention time=1.73 min; ESI-MS(+) m/z [M+2H]²⁺: 1054.2.

### Preparation of Compound 2266

Compound 2266 was prepared on a 30 μmol scale. The yield of the product was 5.1 mg, and its estimated purity by LCMS analysis was 97.1%. Analysis condition B: Retention time=1.98 min; ESI-MS(+) m/z [M+H]⁺: 1901.2.

### Preparation of Example 2267

Example 2267 was prepared on a 200 μmol scale. The yield of the product was 109.7 mg, and its estimated purity by LCMS analysis was 92.6%. Analysis condition B: Retention time=1.66 min; ESI-MS(+) m/z [M+2H]²⁺: 1103.

### Preparation of Example 2268

Example 2268 was prepared on a 50 μmol scale. The yield of the product was 13.4 mg, and its estimated purity by LCMS analysis was 98.9%. Analysis condition B: Retention time=1.79 min; ESI-MS(+) m/z [M+2H]²⁺: 1112.3.

### Preparation of Example 2269

Example 2269 was prepared on a 100 μmol scale. The yield of the product was 41.6 mg, and its estimated purity by LCMS analysis was 86.5%. Analysis condition A: Retention time=1.65 min; ESI-MS(+) m/z [M+2H]²⁺: 1073.4.

### Preparation of Example 2270

Example 2270 was prepared on a 100 μmol scale. The yield of the product was 27.7 mg, and its estimated purity by LCMS analysis was 98%. Analysis condition A: Retention time=1.54 min; ESI-MS(+) m/z [M+2H]²⁺: 1061.4.

### Preparation of Example 2271

Example 2271 was prepared on a 100 μmol scale. The yield of the product was 29.6 mg, and its estimated purity by LCMS analysis was 88.2%. Analysis condition A: Retention time=1.53 min; ESI-MS(+) m/z [M+2H]²⁺: 1046.1.

### Preparation of Example 2272

Example 2272 was prepared on a 100 μmol scale. The yield of the product was 40.8 mg, and its estimated purity by LCMS analysis was 86.1%. Analysis condition A: Retention time=1.62 min; ESI-MS(+) m/z [M+2H]²⁺: 1088.4.

### Preparation of Example 2273

Example 2273 was prepared on a 100 μmol scale. The yield of the product was 15.5 mg, and its estimated purity by LCMS analysis was 95.8%. Analysis condition B: Retention time=1.58 min; ESI-MS(+) m/z [M+2H]²⁺: 1103.5.

### Preparation of Example 2274

Example 2274 was prepared on a 100 μmol scale. The yield of the product was 46.6 mg, and its estimated purity by LCMS analysis was 84.5%. Analysis condition A: Retention time=1.56 min; ESI-MS(+) m/z [M+2H]²⁺: 1076.4.

### Preparation of Example 2275

Example 2275 was prepared on a 50 μmol scale. The yield of the product was 17.2 mg, and its estimated purity by LCMS analysis was 88.9%. Analysis condition A: Retention time=1.5 min; ESI-MS(+) m/z [M+2H]²⁺: 1111.8.

### Preparation of Example 2276

Example 2276 was prepared on a 50 μmol scale. The yield of the product was 13.5 mg, and its estimated purity by LCMS analysis was 89.7%. Analysis condition B: Retention time=1.53 min; ESI-MS(+) m/z [M+2H]²⁺: 1060.7.

### Preparation of Example 2277

Example 2277 was prepared on a 50 μmol scale. The yield of the product was 9.5 mg, and its estimated purity by LCMS analysis was 95.8%. Analysis condition A: Retention time=1.58 min; ESI-MS(+) m/z [M+2H]²⁺: 1075.9.

### Preparation of Example 2278

Example 2278 was prepared on a 50 μmol scale. The yield of the product was 9.3 mg, and its estimated purity by LCMS analysis was 89.9%. Analysis condition A: Retention time=1.64 min; ESI-MS(+) m/z [M+2H]²⁺: 1103.

### Preparation of Example 2279

Example 2279 was prepared on a 50 μmol scale. The yield of the product was 9.8 mg, and its estimated purity by LCMS analysis was 90.6%. Analysis condition B: Retention time=1.58 min; ESI-MS(+) m/z [M+2H]²⁺: 1072.7.

### Preparation of Example 2280

Example 2280 was prepared on a 50 μmol scale. The yield of the product was 2.3 mg, and its estimated purity by LCMS analysis was 82.7%. Analysis condition A: Retention time=1.63 min; ESI-MS(+) m/z [M+2H]²⁺: 1087.6.

### Preparation of Example 2281

Example 2281 was prepared on a 50 μmol scale. The yield of the product was 10.5 mg, and its estimated purity by LCMS analysis was 94.2%. Analysis condition B: Retention time=1.71 min; ESI-MS(+) m/z [M+2H]²⁺: 1102.6.

### Preparation of Example 2282

Example 2282 was prepared on a 50 μmol scale. The yield of the product was 7.7 mg, and its estimated purity by LCMS analysis was 95.6%. Analysis condition B: Retention time=1.58 min; ESI-MS(+) m/z [M+3H]³⁺: 883.7.

### Preparation of Example 2283

Example 2283 was prepared on a 50 μmol scale. The yield of the product was 4.6 mg, and its estimated purity by LCMS analysis was 90.1%. Analysis condition A: Retention time=1.47 min; ESI-MS(+) m/z [M+H]⁺: 1793.7.

### Preparation of Example 2284

Example 2284 was prepared on a 50 μmol scale. The yield of the product was 3.9 mg, and its estimated purity by LCMS analysis was 88.7%. Analysis condition A: Retention time=1.53 min; ESI-MS(+) m/z [M+H]⁺: 1807.6.

### Preparation of Example 2285

Example 2285 was prepared on a 50 μmol scale. The yield of the product was 3.6 mg, and its estimated purity by LCMS analysis was 100%. Analysis condition B: Retention time=1.41 min; ESI-MS(+) m/z [M+2H]²⁺: 1048.5.

### Preparation of Example 2286

Example 2286 was prepared on a 50 μmol scale. The yield of the product was 30.2 mg, and its estimated purity by LCMS analysis was 98.3%. Analysis condition A: Retention time=1.24 min; ESI-MS(+) m/z [M+2H]²⁺: 1084.4.

### Preparation of Example 2287

Example 2287 was prepared on a 50 μmol scale. The yield of the product was 6.2 mg, and its estimated purity by LCMS analysis was 90.4%. Analysis condition B: Retention time=1.38 min; ESI-MS(+) m/z [M+2H]²⁺: 1055.8.

### Preparation of Example 2288

Example 2288 was prepared on a 50 μmol scale. The yield of the product was 16.7 mg, and its estimated purity by LCMS analysis was 86.6%. Analysis condition A: Retention time=1.19 min; ESI-MS(+) m/z [M+2H]²⁺: 1081.8.

### Preparation of Example 2289

Example 2289 was prepared on a 50 μmol scale. The yield of the product was 33.2 mg, and its estimated purity by LCMS analysis was 100%. Analysis condition A: Retention time=1.29 min; ESI-MS(+) m/z [M+2H]²⁺: 1091.5.

### Preparation of Example 2290

Example 2290 was prepared on a 50 μmol scale. The yield of the product was 15.5 mg, and its estimated purity by LCMS analysis was 90.7%. Analysis condition B: Retention time=1.83 min; ESI-MS(+) m/z [M+2H]²⁺: 1039.3.

### Preparation of Example 2291

Example 2291 was prepared on a 50 μmol scale. The yield of the product was 9.9 mg, and its estimated purity by LCMS analysis was 100%. Analysis condition A: Retention time=1.5 min; ESI-MS(+) m/z [M+2H]²⁺: 1097.5.

### Preparation of Example 2292

Example 2292 was prepared on a 50 μmol scale. The yield of the product was 8.5 mg, and its estimated purity by LCMS analysis was 90.8%. Analysis condition A: Retention time=1.53 min; ESI-MS(+) m/z [M+2H]²⁺: 1075.2.

### Preparation of Example 2293

Example 2293 was prepared on a 50 μmol scale. The yield of the product was 10.2 mg, and its estimated purity by LCMS analysis was 100%. Analysis condition A: Retention time=1.49 min; ESI-MS(+) m/z [M+2H]²⁺: 1075.8.

### Preparation of Example 2294

Example 2294 was prepared on a 50 μmol scale. The yield of the product was 16.7 mg, and its estimated purity by LCMS analysis was 91.6%. Analysis condition A: Retention time=1.55 min; ESI-MS(+) m/z [M+2H]²⁺: 1067.2.

### Preparation of Example 2295

Example 2295 was prepared on a 50 μmol scale. The yield of the product was 9.8mg, and its estimated purity by LCMS analysis was 93.8%. Analysis condition A: Retention time =1.55 min; ESI-MS(+) m/z [M2H]²⁺: 1097.7.

### Preparation of Example 2296

Example 2296 was prepared on a 50 μmol scale. The yield of the product was 12.5 mg, and its estimated purity by LCMS analysis was 91.1%. Analysis condition A: Retention time=1.53 min; ESI-MS(+) m/z [M+2H]²⁺: 1067.4.

### Preparation of Example 2297

Example 2297 was prepared on a 50 μmol scale. The yield of the product was 9.4 mg, and its estimated purity by LCMS analysis was 92.500. Analysis condition A: Retention time=1.54 min; ESI-MS(+) m/z [M+2H]²⁺: 1065.4.

### Preparation of Example 2298

Example 2298 was prepared on a 50 μmol scale. The yield of the product was 10.3 mg, and its estimated purity by LCMS analysis was 92.5%. Analysis condition A: Retention time=1.58 min; ESI-MS(+) m/z [M+2H]²⁺: 1065.3.

### Preparation of Example 2299

Example 2299 was prepared on a 50 μmol scale. The yield of the product was 8.3 mg, and its estimated purity by LCMS analysis was 95.5%. Analysis condition A: Retention time=1.57 min; ESI-MS(+) m/z [M+2H]²⁺: 1076.3.

### Preparation of Example 2300

Example 2300 was prepared on a 50 μmol scale. The yield of the product was 15.2 mg, and its estimated purity by LCMS analysis was 92.1%. Analysis condition A: Retention time=1.53 min; ESI-MS(+) m/z [M+2H]²⁺: 1058.3.

### Preparation of Example 2301

Example 2301 was prepared on a 50 μmol scale. The yield of the product was 13.6 mg, and its estimated purity by LCMS analysis was 91.1%. Analysis condition A: Retention time=1.46 min; ESI-MS(+) m/z [M+2H]²⁺: 1066.3.

### HPLC Analysis Conditions for Example 5001-5298:

#1: Column: XBridge C18, 2.1 mm×50 mm, Mobile Phase A: ACN/H₂O (5:95) with 10 mM Ammonium Acetate; Mobile Phase B: ACN/H₂O (95:5) with 10 mM Ammonium Acetate; Temperature: 50° C.; Gradient: 0-100% B (0.0-3.0 min), 100% B (3.0-3.5 min); Flow: 1.0 mL/min; Detection: UV (220 nm) and MS (ESI +).

#2: Column: XBridge C18, 2.1 mm×50 mm, Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI +).

### Preparation of Example 5001

Example 5001 was prepared on a 40 μmol scale. The yield of the product was 10.1 mg, and its estimated purity by LCMS analysis was 95.1%. Analysis condition 2: Retention time=1.72 min; ESI-MS(+) m/z (M+3H)³⁺: 1870.2.

### Preparation of Example 5003

Example 5003 was prepared on a 50 μmol scale. The yield of the product was 33.7 mg, and its estimated purity by LCMS analysis was 98.4%. Analysis condition 2: Retention time=1.73, 1.77 min; ESI-MS(+) m/z (M+3H)³⁺: 1052.1.

### Preparation of Example 5004

Example 5004 was prepared on a 50 μmol scale. The yield of the product was 32.5 mg, and its estimated purity by LCMS analysis was 95.3%. Analysis condition 1: Retention time=1.81 min; ESI-MS(+) m/z (M+3H)³⁺: 1137.

### Preparation of Example 5005

Example 5005 was prepared on a 50 μmol scale. The yield of the product was 13.4 mg, and its estimated purity by LCMS analysis was 95.1%. Analysis condition 2: Retention time=1.65 min; ESI-MS(+) m/z (M+3H)³⁺: 1039.2.

### Preparation of Example 5006

Example 5006 was prepared on a 50 μmol scale. The yield of the product was 3 mg, and its estimated purity by LCMS analysis was 96.8%. Analysis condition 2: Retention time=1.68 min; ESI-MS(+) m/z (M+3H)³⁺: 1045.

### Preparation of Example 5007

Example 5007 was prepared on a 50 μmol scale. The yield of the product was 21 mg, and its estimated purity by LCMS analysis was 99.2%. Analysis condition 1: Retention time=1.69 min; ESI-MS(+) m/z (M+3H)³⁺: 1100.

### Preparation of Example 5008

Example 5008 was prepared on a 50 μmol scale. The yield of the product was 29.6 mg, and its estimated purity by LCMS analysis was 96.6%. Analysis condition 1: Retention time=1.64 min; ESI-MS(+) m/z (M+3H)³⁺: 1119.2.

### Preparation of Example 5009

Example 5009 was prepared on a 30 μmol scale. The yield of the product was 12.7 mg, and its estimated purity by LCMS analysis was 96.3%. Analysis condition 2: Retention time=1.88 min; ESI-MS(+) m/z (M+3H)³⁺: 1976.2.

### Preparation of Example 5013

Example 5013 was prepared on a 50 μmol scale. The yield of the product was 0.9 mg, and its estimated purity by LCMS analysis was 71.7%. Analysis condition 1: Retention time=1.66 min; ESI-MS(+) m/z (M+3H)³⁺: 1088.2.

### Preparation of Example 5014

Example 5014 was prepared on a 50 μmol scale. The yield of the product was 1.8 mg, and its estimated purity by LCMS analysis was 92.8%. Analysis condition 1: Retention time=1.65 min: ESI-MS(+) m/z (M+3H)³⁺: 1088.3.

### Preparation of Example 5015

Example 5015 was prepared on a 50 μmol scale. The yield of the product was 5.7 mg, and its estimated purity by LCMS analysis was 96.4%. Analysis condition 1: Retention time=1.5 min; ESI-MS(+) m/z (M+3H)³⁺: 1065.6.

### Preparation of Example 5016

Example 5016 was prepared on a 50 μmol scale. The yield of the product was 0.8 mg, and its estimated purity by LCMS analysis was 100%. Analysis condition 2: Retention time=1.35 min; ESI-MS(+) m/z (M+3H)³⁺: 1066.6.

### Preparation of Example 5017

Example 5017 was prepared on a 50 μmol scale. The yield of the product was 3.3 mg, and its estimated purity by LCMS analysis was 93.9%. Analysis condition 2: Retention time=1.63 min: ESI-MS(+) m/z (M+3H)³⁺: 1128.

### Preparation of Example 5018

Example 5018 was prepared on a 50 μmol scale. The yield of the product was 6.7 mg, and its estimated purity by LCMS analysis was 90.9%. Analysis condition 1: Retention time=1.6 min; ESI-MS(+) m/z (M+3H)³⁺: 1128.1.

### Preparation of Example 5019

Example 5019 was prepared on a 50 μmol scale. The yield of the product was 7.2 mg, and its estimated purity by LCMS analysis was 98.8%. Analysis condition 2: Retention time=1.83 min; ESI-MS(+) m/z (M+3H)³⁺: 1106.

### Preparation of Example 5020

Example 5020 was prepared on a 50 μmol scale. The yield of the product was 12.5 mg, and its estimated purity by LCMS analysis was 90.1%. Analysis condition 2: Retention time=1.68 min: ESI-MS(+) m/z (M+3H)³⁺: 1106.

### Preparation of Example 5021

Example 5021 was prepared on a 50 μmol scale. The yield of the product was 3.9 mg, and its estimated purity by LCMS analysis was 100%. Analysis condition 1: Retention time=1.64 min; ESI-MS(+) m/z (M+3H)³⁺: 1087.9.

### Preparation of Example 5022

Example 5022 was prepared on a 50 μmol scale. The yield of the product was 6.3 mg, and its estimated purity by LCMS analysis was 94.5%. Analysis condition 2: Retention time=1.92 min; ESI-MS(+) m/z (M+3H)³⁺: 1134.1.

### Preparation of Example 5023

Example 5023 was prepared on a 50 μmol scale. The yield of the product was 3.9 mg, and its estimated purity by LCMS analysis was 100%. Analysis condition 1: Retention time=1.74 min; ESI-MS(+) m/z (M+3H)³⁺: 1130.8.

### Preparation of Example 5024

Example 5024 was prepared on a 50 μmol scale. The yield of the product was 6.5 mg, and its estimated purity by LCMS analysis was 97.2%. Analysis condition 1: Retention time=1.67 min; ESI-MS(+) m/z (M+3H)³⁺: 1095.

### Preparation of Example 5025

Example 5025 was prepared on a 50 μmol scale. The yield of the product was 18.1 mg, and its estimated purity by LCMS analysis was 80.1%. Analysis condition 2: Retention time=2.03 min; ESI-MS(+) m/z (M+3H)³⁺: 1874.6.

### Preparation of Example 5026

Example 5026 was prepared on a 50 μmol scale. The yield of the product was 3.4 mg, and its estimated purity by LCMS analysis was 95.5%. Analysis condition 2: Retention time=1.85 min; ESI-MS(+) m/z (M+3H)³⁺: 1902.5.

### Preparation of Example 5027

Example 5027 was prepared on a 900 μmol scale. The yield of the product was 105.6 mg, and its estimated purity by LCMS analysis was 87.7%. Analysis condition 2: Retention time=1.69 min: ESI-MS(+) m/z (M+3H)³⁺: 1102.4.

### Preparation of Example 5028

Example 5028 was prepared on a 900 μmol scale. The yield of the product was 149.5 mg, and its estimated purity by LCMS analysis was 83.4%. Analysis condition 2: Retention time=1.64 min; ESI-MS(+) m/z (M+3H)³⁺: 1102.6.

### Preparation of Example 5029

Example 5029 was prepared on a 50 μmol scale. The yield of the product was 0.8 mg, and its estimated purity by LCMS analysis was 100%. Analysis condition 1: Retention time=1.48 min; ESI-MS(+) m/z (M+3H)³⁺: 1083.6.

### Preparation of Example 5030

Example 5030 was prepared on a 50 μmol scale. The yield of the product was 29.5 mg, and its estimated purity by LCMS analysis was 91.2%. Analysis condition 2: Retention time=1.72 min; ESI-MS(+) m/z (M+3H)³⁺: 1052.1.

### Preparation of Example 5031

Example 5031 was prepared on a 50 μmol scale. The yield of the product was 6.4 mg, and its estimated purity by LCMS analysis was 90.6%. Analysis condition 2: Retention time=1.87 min; ESI-MS(+) m/z (M+3H)³⁺: 1091.4.

### Preparation of Example 5032

Example 5032 was prepared on a 50 μmol scale. The yield of the product was 5.7 mg, and its estimated purity by LCMS analysis was 906%. Analysis condition 1: Retention time=1.94 min; ESI-MS(+) m/z (M+3H)³⁺: 1932.7.

### Preparation of Example 5033

Example 5033 was prepared on a 50 μmol scale. The yield of the product was 29.9 mg, and its estimated purity by LCMS analysis was 91.3%. Analysis condition 2: Retention time=1.55 min; ESI-MS(+) m/z (M+3H)³⁺: 1092.4.

### Preparation of Example 5034

Example 5034 was prepared on a 50 μmol scale. The yield of the product was 9.8 mg, and its estimated purity by LCMS analysis was 93.6%. Analysis condition 2: Retention time=1.67 min; ESI-MS(+) m/z (M+3H)³⁺: 1137.4.

### Preparation of Example 5035

Example 5035 was prepared on a 50 μmol scale. The yield of the product was 11.9 mg, and its estimated purity by LCMS analysis was 90.2%. Analysis condition 1: Retention time=1.56 min; ESI-MS(+) m/z (M+3H)³⁺: 1132.6.

### Preparation of Example 5036

Example 5036 was prepared on a 600 μmol scale. The yield of the product was 277.7 mg, and its estimated purity by LCMS analysis was 92.3%. Analysis condition 2: Retention time=1.66 min; ESI-MS(+) m/z (M+3H)³⁺: 1119.9.

### Preparation of Example 5037

Example 5037 was prepared on a 50 μmol scale. The yield of the product was 0.6 mg, and its estimated purity by LCMS analysis was 100%. Analysis condition 2: Retention time=1.78 min; ESI-MS(+) m/z (M+3H)³⁺: 1058.9.

### Preparation of Example 5038

Example 5038 was prepared on a 50 μmol scale. The yield of the product was 7 mg, and its estimated purity by LCMS analysis was 92.4%. Analysis condition 1: Retention time=1.42 min; ESI-MS(+) m/z (M+3H)³⁺: 1058.8.

### Preparation of Example 5039

Example 5039 was prepared on a 50 μmol scale. The yield of the product was 38.2 mg, and its estimated purity by LCMS analysis was 90.3%. Analysis condition 2: Retention time=1.4 min; ESI-MS(+) m/z (M+3H)³⁺: 1058.9.

### Preparation of Example 5040

Example 5040 was prepared on a 50 μmol scale. The yield of the product was 17.8 mg, and its estimated purity by LCMS analysis was 93.1%. Analysis condition 2: Retention time=1.42 min; ESI-MS(+) m/z (M+3H)³⁺: 1066.

### Preparation of Example 5041

Example 5041 was prepared on a 50 μmol scale. The yield of the product was 17.2 mg, and its estimated purity by LCMS analysis was 92.7%. Analysis condition 1: Retention time=1.58 min; ESI-MS(+) m/z (M+3H)³⁺: 1092.7.

### Preparation of Example 5042

Example 5042 was prepared on a 50 μmol scale. The yield of the product was 14 mg, and its estimated purity by LCMS analysis was 93.6%. Analysis condition 1: Retention time=1.58 min; ESI-MS(+) m/z (M+3H)³⁺: 1092.8.

### Preparation of Example 5043

Example 5043 was prepared on a 50 μmol scale. The yield of the product was 37.7 mg, and its estimated purity by LCMS analysis was 95.7%. Analysis condition 2: Retention time=1.49 min; ESI-MS(+) m/z (M+3H)³⁺: 1073.4.

### Preparation of Example 5044

Example 5044 was prepared on a 50 μmol scale. The yield of the product was 45.5 mg, and its estimated purity by LCMS analysis was 90.3%. Analysis condition 2: Retention time=1.58 min; ESI-MS(+) m/z (M+3H)³⁺: 1083.5.

### Preparation of Example 5045

Example 5045 was prepared on a 50 μmol scale. The yield of the product was 25.1 mg, and its estimated purity by LCMS analysis was 94.1%. Analysis condition 1: Retention time=1.56 min: ESI-MS(+) m/z (M+3H)³⁺: 1092.9.

### Preparation of Example 5046

Example 5046 was prepared on a 50 μmol scale. The yield of the product was 14.3 mg, and its estimated purity by LCMS analysis was 96.3%. Analysis condition 1: Retention time=1.57 min; ESI-MS(+) m/z (M+3H)³⁺: 1131.3.

### Preparation of Example 5047

Example 5047 was prepared on a 34 μmol scale. The yield of the product was 6.6 mg, and its estimated purity by LCMS analysis was 93.1%. Analysis condition 2: Retention time=1.65 min; ESI-MS (+) m/z (M+3H)³⁺: 1118.2.

### Preparation of Example 5048

Example 5048 was prepared on a 34 μmol scale. The yield of the product was 4.1 mg, and its estimated purity by LCMS analysis was 92.3%. Analysis condition 2: Retention time=1.58 min; ESI-MS(+) m/z (M+3H)³⁺: 1091.3.

### Preparation of Example 5049

Example 5049 was prepared on a 34 μmol scale. The yield of the product was 2.9 mg, and its estimated purity by LCMS analysis was 93.1%. Analysis condition 1: Retention time=1.53 min; ESI-MS(+) m/z (M+3H)³⁺: 1097.

### Preparation of Example 5050

Example 5050 was prepared on a 34 μmol scale. The yield of the product was 7.7 mg, and its estimated purity by LCMS analysis was 92.9%. Analysis condition 2: Retention time=1.63 min; ESI-MS(+) m/z (M+3H)³⁺: 1090.2.

### Preparation of Example 5051

Example 5051 was prepared on a 34 μmol scale. The yield of the product was 5.3 mg, and its estimated purity by LCMS analysis was 943%. Analysis condition 2: Retention time=1.56 min; ESI-MS(+) m/z (M+3H)³⁺: 1063.3.

### Preparation of Example 5052

Example 5052 was prepared on a 34 μmol scale. The yield of the product was 3.1 mg, and its estimated purity by LCMS analysis was 90.9%. Analysis condition 1: Retention time=1.53 min; ESI-MS(+) m/z (M+3H)³⁺: 1069.

### Preparation of Example 5053

Example 5053 was prepared on a 34 μmol scale. The yield of the product was 6.9 mg, and its estimated purity by LCMS analysis was 90.9%. Analysis condition 2: Retention time=1.71 min; ESI-MS (+) m/z (M+3H)³⁺: 1096.8.

### Preparation of Example 5054

Example 5054 was prepared on a 34 μmol scale. The yield of the product was 3.9 mg, and its estimated purity by LCMS analysis was 91.300. Analysis condition 2: Retention time=1.63 min; ESI-MS(+) m/z (M+3H)³⁺: 1069.8.

### Preparation of Example 5055

Example 5055 was prepared on a 34 μmol scale. The yield of the product was 4.1 mg, and its estimated purity by LCMS analysis was 91.8%. Analysis condition 2: Retention time=1.57 min; ESI-MS(+) m/z (M+3H)³⁺: 1076.

### Preparation of Example 5056

Example 5056 was prepared on a 50 μmol scale. The yield of the product was 8.7 mg, and its estimated purity by LCMS analysis was 93.3%. Analysis condition 2: Retention time=1.56 min; ESI-MS(+) m/z (M+3H)³⁺: 1078.7.

### Preparation of Example 5057

Example 5057 was prepared on a 50 μmol scale. The yield of the product was 6.6 mg, and its estimated purity by LCMS analysis was 90.8%. Analysis condition 2: Retention time=1.45 min; ESI-MS(+) m/z (M+3H)³⁺: 1072.2.

### Preparation of Example 5058

Example 5058 was prepared on a 34 μmol scale. The yield of the product was 4.2 mg, and its estimated purity by LCMS analysis was 9400. Analysis condition 1: Retention time=1.62 min; ESI-MS(+) m/z (M+3H)³⁺: 1111.3.

### Preparation of Example 5059

Example 5059 was prepared on a 34 μmol scale. The yield of the product was 5 mg, and its estimated purity by LCMS analysis was 100%. Analysis condition 1: Retention time=1.64 min; ESI-MS(+) m/z (M+3H)³⁺: 1084.6.

### Preparation of Example 5060

Example 5060 was prepared on a 34 μmol scale. The yield of the product was 4.7 mg, and its estimated purity by LCMS analysis was 92.7%. Analysis condition 2: Retention time=1.38 min; ESI-MS(+) m/z (M+3H)³⁺: 1090.8.

### Preparation of Example 5061

Example 5061 was prepared on a 34 μmol scale. The yield of the product was 2.9 mg, and its estimated purity by LCMS analysis was 97.3%. Analysis condition 1: Retention time=1.57 min; ESI-MS(+) m/z (M+3H)³⁺: 1056.4.

### Preparation of Example 5062

Example 5062 was prepared on a 34 μmol scale. The yield of the product was 4.3 mg, and its estimated purity by LCMS analysis was 90.3%. Analysis condition 2: Retention time=1.39 min; ESI-MS(+) m/z (M+3H)³⁺: 1062.4.

### Preparation of Example 5063

Example 5063 was prepared on a 34 μmol scale. The yield of the product was 2.4 mg, and its estimated purity by LCMS analysis was 100%. Analysis condition 1: Retention time=1.7 min; ESI-MS(+) m/z (M+3H)³⁺: 1062.9.

### Preparation of Example 5064

Example 5064 was prepared on a 34 μmol scale. The yield of the product was 6.9 mg, and its estimated purity by LCMS analysis was 93.5%. Analysis condition 2: Retention time=1.48 min; ESI-MS(+) m/z (M+3H)³⁺: 1069.1.

### Preparation of Example 5065

Example 5065 was prepared on a 50 μmol scale. The yield of the product was 39.6 mg, and its estimated purity by LCMS analysis was 93.7%. Analysis condition 2: Retention time=1.61 min; ESI-MS(+) m/z (M+3H)³⁺: 1080.6.

### Preparation of Example 5066

Example 5066 was prepared on a 50 μmol scale. The yield of the product was 23.8 mg, and its estimated purity by LCMS analysis was 98.4%. Analysis condition 2: Retention time=1.55 min; ESI-MS(+) m/z (M+3H)³⁺: 1073.7.

### Preparation of Example 5067

Example 5067 was prepared on a 50 μmol scale. The yield of the product was 32 mg, and its estimated purity by LCMS analysis was 96.3%. Analysis condition 1: Retention time=1.75 min: ESI-MS(+) m/z (M+3H)³⁺: 1074.5.

### Preparation of Example 5068

Example 5068 was prepared on a 50 μmol scale. The yield of the product was 37.2 mg, and its estimated purity by LCMS analysis was 95.5%. Analysis condition 1: Retention time=1.66 min; ESI-MS(+) m/z (M+3H)³⁺: 1086.6.

### Preparation of Example 5069

Example 5069 was prepared on a 50 μmol scale. The yield of the product was 26.5 mg, and its estimated purity by LCMS analysis was 96.5%. Analysis condition 1: Retention time=1.8 min; ESI-MS(+) m/z (M+3H)³⁺: 1109.8.

### Preparation of Example 5070

Example 5070 was prepared on a 50 μmol scale. The yield of the product was 30.7 mg, and its estimated purity by LCMS analysis was 95.5%. Analysis condition 2: Retention time=1.6 min: ESI-MS(+) m/z (M+3H)³⁺: 1103.6.

### Preparation of Example 5071

Example 5071 was prepared on a 50 μmol scale. The yield of the product was 35.1 mg, and its estimated purity by LCMS analysis was 95.7%. Analysis condition 2: Retention time=1.59 min; ESI-MS(+) m/z (M+3H)³⁺: 1103.7.

### Preparation of Example 5072

Example 5072 was prepared on a 50 μmol scale. The yield of the product was 56 mg, and its estimated purity by LCMS analysis was 94.7%. Analysis condition 1: Retention time=1.63 min; ESI-MS(+) m/z (M+3H)³⁺: 744.5.

### Preparation of Example 5073

Example 5073 was prepared on a 50 μmol scale. The yield of the product was 51.9 mg, and its estimated purity by LCMS analysis was 96%. Analysis condition 2: Retention time=1.72 min: ESI-MS(+) m/z (M+3H)³⁺: 1128.2.

### Preparation of Example 5074

Example 5074 was prepared on a 50 μmol scale. The yield of the product was 27.6 mg, and its estimated purity by LCMS analysis was 97.400. Analysis condition 1: Retention time=1.96 min; ESI-MS(+) m/z (M+3H)³⁺: 1159.1.

### Preparation of Example 5075

Example 5075 was prepared on a 50 μmol scale. The yield of the product was 2.1 mg, and its estimated purity by LCMS analysis was 96%. Analysis condition 1: Retention time=1.57 min; ESI-MS(+) m/z (M+3H)³⁺: 1108.8.

### Preparation of Example 5076

Example 5076 was prepared on a 50 μmol scale. The yield of the product was 1.6 mg, and its estimated purity by LCMS analysis was 89.2%. Analysis condition 2: Retention time=1.58 min; ESI-MS(+) m/z (M+3H)³⁺: 1152.5.

### Preparation of Example 5077

Example 5077 was prepared on a 50 μmol scale. The yield of the product was 4.7 mg, and its estimated purity by LCMS analysis was 93.8%. Analysis condition 2: Retention time=1.64 min ESI-MS(+) m/z (M+3H)³⁺: 1152.1.

### Preparation of Example 5078

Example 5078 was prepared on a 50 μmol scale. The yield of the product was 4.6 mg, and its estimated purity by LCMS analysis was 95.1%. Analysis condition 1: Retention time=1.75 min; ESI-MS(+) m/z (M+3H)³⁺: 1161.3.

### Preparation of Example 5079

Example 5079 was prepared on a 50 μmol scale. The yield of the product was 18.5 mg, and its estimated purity by LCMS analysis was 85.4%. Analysis condition 2: Retention time=1.65 min; ESI-MS(+) m/z (M+3H)³⁺: 1152.1.

### Preparation of Example 5080

Example 5080 was prepared on a 50 μmol scale. The yield of the product was 20.3 mg, and its estimated purity by LCMS analysis was 85.5%. Analysis condition 2: Retention time=1.62 min; ESI-MS(+) m/z (M+3H)³⁺: 1160.4.

### Preparation of Example 5081

Example 5081 was prepared on a 50 μmol scale. The yield of the product was 6.1 mg, and its estimated purity by LCMS analysis was 95.4%. Analysis condition 1: Retention time=1.74 min; ESI-MS(+) m/z (M+3H)³⁺: 1169.2.

### Preparation of Example 5082

Example 5082 was prepared on a 50 μmol scale. The yield of the product was 6.5 mg, and its estimated purity by LCMS analysis was 96.2%. Analysis condition 1: Retention time=1.75 min; ESI-MS(+) m/z (M+3H)³⁺: 1178.9.

### Preparation of Example 5083

Example 5083 was prepared on a 50 μmol scale. The yield of the product was 1.4 mg, and its estimated purity by LCMS analysis was 88.4%. Analysis condition 1: Retention time=1.7 min; ESI-MS(+) m/z (M+3H)³⁺: 1179.2.

### Preparation of Example 5084

Example 5084 was prepared on a 50 μmol scale. The yield of the product was 19.3 mg, and its estimated purity by LCMS analysis was 96.9%. Analysis condition 2: Retention time=1.67 min; ESI-MS(+) m/z (M+3H)³⁺: 1188.7.

### Preparation of Example 5085

Example 5085 was prepared on a 50 μmol scale. The yield of the product was 20.7 mg, and its estimated purity by LCMS analysis was 95.9%. Analysis condition 2: Retention time=1.47 min: ESI-MS(+) m/z (M+3H)³⁺: 1182.6.

### Preparation of Example 5086

Example 5086 was prepared on a 50 μmol scale. The yield of the product was 12.3 mg, and its estimated purity by LCMS analysis was 94.5%. Analysis condition 2: Retention time=1.56 min; ESI-MS(+) m/z (M+3H)³⁺: 1174.9.

### Preparation of Example 5087

Example 5087 was prepared on a 50 mol scale. The yield of the product was 24.8 mg, and its estimated purity by LCMS analysis was 94.7%. Analysis condition 2: Retention time=1.54 min; ESI-MS(+) m/z (M+3H)³⁺: 1174.9.

### Preparation of Example 5088

Example 5088 was prepared on a 50 μmol scale. The yield of the product was 18.1 mg, and its estimated purity by LCMS analysis was 95.4%. Analysis condition 2: Retention time=1.62 min; ESI-MS(+) m/z (M+3H)³⁺: 1121.

### Preparation of Example 5089

Example 5089 was prepared on a 50 μmol scale. The yield of the product was 17.2 mg, and its estimated purity by LCMS analysis was 96.8%. Analysis condition 2: Retention time=1.64 min; ESI-MS(+) m/z (M+3H)³⁺: 1128.5.

### Preparation of Example 5090

Example 5090 was prepared on a 50 μmol scale. The yield of the product was 14.3 mg, and its estimated purity by LCMS analysis was 96%. Analysis condition 2: Retention time=1.64 min; ESI-MS(+) m/z (M+3H)³⁺: 1121.

### Preparation of Example 5091

Example 5091 was prepared on a 50 μmol scale. The yield of the product was 27.2 mg, and its estimated purity by LCMS analysis was 97.4%. Analysis condition 2: Retention time=1.65 min ESI-MS(+) m/z (M+3H)³⁺: 1149.

### Preparation of Example 5092

Example 5092 was prepared on a μmol scale. The yield of the product was 2.8 mg, and its estimated purity by LCMS analysis was 91.6%. Analysis condition 2: Retention time=1.52 min; ESI-MS(+) m/z (M+3H)³⁺: 1168.3.

### Preparation of Example 5093

Example 5093 was prepared on a μmol scale. The yield of the product was 1.6 mg, and its estimated purity by LCMS analysis was 95.2%. Analysis condition 1: Retention time=1.74 min; ESI-MS(+) m/z (M+3H)³⁺: 1161.6.

### Preparation of Example 5094

Example 5094 was prepared on a 50 μmol scale. The yield of the product was 10.1 mg, and its estimated purity by LCMS analysis was 99.1%. Analysis condition 2: Retention time=1.87 min; ESI-MS(+) m/z (M+3H)³⁺: 1097.

### Preparation of Example 5095

Example 5095 was prepared on a 50 μmol scale. The yield of the product was 36.6 mg, and its estimated purity by LCMS analysis was 95.2%. Analysis condition 2: Retention time=1.75 min; ESI-MS(+) m/z (M+3H)³⁺: 1089.5.

### Preparation of Example 5096

Example 5096 was prepared on a 50 μmol scale. The yield of the product was 23.3 mg, and its estimated purity by LCMS analysis was 98%. Analysis condition 2: Retention time=1.72 min; ESI-MS(+) m/z (M+3H)³⁺: 1089.6.

### Preparation of Example 5097

Example 5097 was prepared on a 50 μmol scale. The yield of the product was 35 mg, and its estimated purity by LCMS analysis was 96.8%. Analysis condition 1: Retention time=1.67 min; ESI-MS(+) m/z (M+3H)³⁺: 1128.

### Preparation of Example 5098

Example 5098 was prepared on a 50 μmol scale. The yield of the product was 10.8 mg, and its estimated purity by LCMS analysis was 98.5%. Analysis condition 2: Retention time=1.66 min; ESI-MS(+) m/z (M+3H)³⁺: 1120.9.

### Preparation of Example 5099

Example 5099 was prepared on a 50 μmol scale. The yield of the product was 14 mg, and its estimated purity by LCMS analysis was 95.8%. Analysis condition 2: Retention time=1.66 min: ESI-MS(+) m/z (M+3H)³⁺: 1166.7.

### Preparation of Example 5100

Example 5100 was prepared on a 9.1 μmol scale. The yield of the product was 0.6 mg, and its estimated purity by LCMS analysis was 95.300. Analysis condition 2: Retention time=1.7 min; ESI-MS(+) m/z (M+3H)³⁺: 1085.9.

### Preparation of Example 5101

Example 5101 was prepared on a 50 μmol scale. The yield of the product was 26.7 mg, and its estimated purity by LCMS analysis was 97.2%. Analysis condition 1: Retention time=1.77 min; ESI-MS(+) m/z (M+3H)³⁺: 1076.4.

### Preparation of Example 5102

Example 5102 was prepared on a 50 μmol scale. The yield of the product was 11.5 mg, and its estimated purity by LCMS analysis was 100%. Analysis condition 2: Retention time=1.62 min: ESI-MS(+) m/z (M+3H)³⁺: 1134.6.

### Preparation of Example 5103

Example 5103 was prepared on a 9.1 μmol scale. The yield of the product was 0.6 mg, and its estimated purity by LCMS analysis was 96.1%. Analysis condition 2: Retention time=1.72 min; ESI-MS(+) m/z (M+3H)³⁺: 1085.4.

### Preparation of Example 5104

Example 5104 was prepared on a 9.1 μmol scale. The yield of the product was 1 mg, and its estimated purity by LCMS analysis was 90%. Analysis condition 2: Retention time=1.75 min; ESI-MS(+) m/z (M+3H)³⁺: 1085.5.

### Preparation of Example 5105

Example 5105 was prepared on a 50 μmol scale. The yield of the product was 2.3 mg, and its estimated purity by LCMS analysis was 90.9%. Analysis condition 2: Retention time=1.69 min; ESI-MS(+) m/z (M+3H)³⁺: 761.1.

### Preparation of Example 5106

Example 5106 was prepared on a 7.7 μmol scale. The yield of the product was 2.2 mg, and its estimated purity by LCMS analysis was 91.6%. Analysis condition 1: Retention time=1.85 min; ESI-MS(+) m/z (M+3H)³⁺: 1152.2.

### Preparation of Example 5107

Example 5107 was prepared on a 9.5 μmol scale. The yield of the product was 6.9 mg, and its estimated purity by LCMS analysis was 90.5%. Analysis condition 2: Retention time=1.9 min; ESI-MS(+) m/z (M+3H)³⁺: 1151.9.

### Preparation of Example 5108

Example 5108 was prepared on a 9.5 μmol scale. The yield of the product was 8.2 mg, and its estimated purity by LCMS analysis was 90.1%. Analysis condition 1: Retention time=1.77 min; ESI-MS(+) m/z (M+3H)³⁺: 1124.1.

### Preparation of Example 5109

Example 5109 was prepared on a 50 μmol scale. The yield of the product was 67.9 mg, and its estimated purity by LCMS analysis was 95%. Analysis condition 2: Retention time=1.62 min; ESI-MS(+) m/z (M+3H)³⁺: 1121.

### Preparation of Example 5110

Example 5110 was prepared on a 50 μmol scale. The yield of the product was 66.8 mg, and its estimated purity by LCMS analysis was 93.9%. Analysis condition 1: Retention time=1.46 min; ESI-MS(+) m/z (M+3H)³⁺: 1120.5.

### Preparation of Example 5111

Example 5111 was prepared on a 50 μmol scale. The yield of the product was 30.4 mg, and its estimated purity by LCMS analysis was 96.1%. Analysis condition 2: Retention time=1.74 min: ESI-MS(+) m/z (M+3H)³⁺: 1111.

### Preparation of Example 5112

Example 5112 was prepared on a 50 μmol scale. The yield of the product was 33.2 mg, and its estimated purity by LCMS analysis was 96%. Analysis condition 1: Retention time=1.72 min; ESI-MS(+) m/z (M+3H)³⁺: 1159.6.

### Preparation of Example 5113

Example 5113 was prepared on a 50 μmol scale. The yield of the product was 12.3 mg, and its estimated purity by LCMS analysis was 100%. Analysis condition 2: Retention time=1.75 min; ESI-MS(+) m/z (M+3H)³⁺: 1129.4.

### Preparation of Example 5114

Example 5114 was prepared on a 50 μmol scale. The yield of the product was 63.9 mg, and its estimated purity by LCMS analysis was 96.2%. Analysis condition 2: Retention time=1.75 min; ESI-MS(+) m/z (M+3H)³⁺: 1165.7.

### Preparation of Example 5115

Example 5115 was prepared on a 50 μmol scale. The yield of the product was 52.5 mg, and its estimated purity by LCMS analysis was 96.6%. Analysis condition 2: Retention time=1.68 min; ESI-MS(+) m/z (M+3H)³⁺: 1159.6.

### Preparation of Example 5116

Example 5116 was prepared on a 50 μmol scale. The yield of the product was 4 mg, and its estimated purity by LCMS analysis was 95.3%. Analysis condition 1: Retention time=1.85 min; ESI-MS(+) m/z (M+3H)³⁺: 1208.6.

### Preparation of Example 5117

Example 5117 was prepared on a μmol scale. The yield of the product was 24.2 mg, and its estimated purity by LCMS analysis was 91.1%. Analysis condition 2: Retention time=1.54 min; ESI-MS(+) m/z (M+3H)³⁺: 1166.

### Preparation of Example 5118

Example 5118 was prepared on a 50 μmol scale. The yield of the product was 21 mg, and its estimated purity by LCMS analysis was 98.4%. Analysis condition 1: Retention time=1.7 min; ESI-MS(+) m/z (M+3H)³⁺: 752.7.

### Preparation of Example 5119

Example 5119 was prepared on a 50 μmol scale. The yield of the product was 40.7 mg, and its estimated purity by LCMS analysis was 92.9%. Analysis condition 1: Retention time=1.56 min; ESI-MS(+) m/z (M+3H)³⁺: 1071.5.

### Preparation of Example 5120

Example 5120 was prepared on a 50 μmol scale. The yield of the product was 8.6 mg, and its estimated purity by LCMS analysis was 97.40. Analysis condition 2: Retention time=1.55 min: ESI-MS(+) m/z (M+3H)³⁺: 1181.9.

### Preparation of Example 5121

Example 5121 was prepared on a mol scale. The yield of the product was 4.8 mg, and its estimated purity by LCMS analysis was 93.3%. Analysis condition 1: Retention time=1.82 min; ESI-MS(+) m/z (M+3H)³⁺: 1124.8.

### Preparation of Example 5122

Example 5122 was prepared on a μmol scale. The yield of the product was 6.9 mg, and its estimated purity by LCMS analysis was 91.4%. Analysis condition 2: Retention time=1.44 min; ESI-MS(+) m/z (M+3H)³⁺: 1167.1.

### Preparation of Example 5123

Example 5123 was prepared on a μmol scale. The yield of the product was 5.5 mg, and its estimated purity by LCMS analysis was 98%. Analysis condition 1: Retention time=1.71 min; ESI-MS(+) m/z (M+3H)³⁺: 1173.7.

### Preparation of Example 5124

Example 5124 was prepared on a μmol scale. The yield of the product was 4.3 mg, and its estimated purity by LCMS analysis was 91.5%. Analysis condition 1: Retention time=1.83 min; ESI-MS(+) m/z (M+3H)³⁺: 1037.9.

### Preparation of Example 5125

Example 5125 was prepared on a μmol scale. The yield of the product was 9.4 mg, and its estimated purity by LCMS analysis was 90.1%. Analysis condition 1: Retention time=1.87 min; ESI-MS(+) m/z (M+3H)³⁺: 1025.4.

### Preparation of Example 5126

Example 5126 was prepared on a μmol scale. The yield of the product was 2.6 mg, and its estimated purity by LCMS analysis was 91.6%. Analysis condition 1: Retention time=1.89 min; ESI-MS(+) m/z (M+3H)³⁺: 1012.5.

### Preparation of Example 5127

Example 5127 was prepared on a μmol scale. The yield of the product was 26.4 and its estimated purity by LCMS analysis was 9%. Analysis condition: Retention time=1.9 min: ES-MS(+m z (M+3H)³⁺: 1189.8.

Example 5128 was prepared on a mol scale. The yield of the product was 6.4 g and its estimated purity by LCMS analysis was 9.9. Analysis condition: Retention time=1.32 min; ES-MS(+) m/z (M+3H)³⁺: 118.

### Preparation of Example 5129

Example 5129 was prepared on a μmol scale. The yield of the product was 2.8 mg, and its estimated purity by LCMS analysis was 96.50%. Analysis condition 1: Retention time=1.9 min; ESI-MS(+) m/z (M+3H)³⁺: 1176.8.

### Preparation of Example 5130

Example 5130 was prepared on a μmol scale. The yield of the product was 5.5 mg, and its estimated purity by LCMS analysis was 90%. Analysis condition 1: Retention time=1.81 min; ESI-MS (+) m/z (M+3H)³⁺: 1170.7.

### Preparation of Example 5131

Example 5131 was prepared on a μmol scale. The yield of the product was 10.6 mg, and its estimated purity by LCMS analysis was 90.6%. Analysis condition 2: Retention time=1.45 min; ESI-MS(+) m/z (M+3H)³⁺: 1145.9.

### Preparation of Example 5132

Example 5132 was prepared on a μmol scale. The yield of the product was 12.2 mg, and its estimated purity by LCMS analysis was 90.9%. Analysis condition 2: Retention time=1.52 min; ESI-MS(+) m/z (M+3H)³⁺: 1167.7.

### Preparation of Example 5133

Example 5133 was prepared on a μmol scale. The yield of the product was 8.4 mg, and its estimated purity by LCMS analysis was 90.5%. Analysis condition 2: Retention time=1.45 min; ESI-MS(+) m/z (M+3H)³⁺: 1173.7.

### Preparation of Example 5134

Example 5134 was prepared on a μmol scale. The yield of the product was 5.4 mg, and its estimated purity by LCMS analysis was 91.2%. Analysis condition 2: Retention time=1.6 min; ESI-MS(+) m/z (M+3H)³⁺: 1195.8.

### Preparation of Example 5135

Example 5135 was prepared on a 50 μmol scale. The yield of the product was 16.5 mg, and its estimated purity by LCMS analysis was 95.3%. Analysis condition 1: Retention time=1.76 min; ESI-MS(+) m/z (M+3H)³⁺: 1035.5.

### Preparation of Example 5136

Example 5136 was prepared on a 50 μmol scale. The yield of the product was 20.4 mg, and its estimated purity by LCMS analysis was 97.9%. Analysis condition 1: Retention time=1.77 min; ESI-MS(+) m/z (M+3H)³⁺: 1042.8.

### Preparation of Example 5137

Example 5137 was prepared on a 50 μmol scale. The yield of the product was 12 mg, and its estimated purity by LCMS analysis was 94.300. Analysis condition 1: Retention time=1.75 min: ESI-MS(+) m/z (M+3H)³⁺: 1057.9.

### Preparation of Example 5138

Example 5138 was prepared on a 50 μmol scale. The yield of the product was 7.6 mg, and its estimated purity by LCMS analysis was 95.8%. Analysis condition 1: Retention time=1.7 min; ESI-MS(+) m/z (M+3H)³⁺: 1058.

### Preparation of Example 5139

Example 5139 was prepared on a 50 μmol scale. The yield of the product was 21.9 mg, and its estimated purity by LCMS analysis was 94.4%. Analysis condition 2: Retention time=1.32 min; ESI-MS(+) m/z (M+3H)³⁺: 1088.1.

### Preparation of Example 5140

Example 5140 was prepared on a 50 μmol scale. The yield of the product was 17.4 mg, and its estimated purity by LCMS analysis was 83.3%. Analysis condition 2: Retention time=1.71 min; ESI-MS(+) m/z (M+3H)³⁺: 1038.6.

### Preparation of Example 5141

Example 5141 was prepared on a 50 μmol scale. The yield of the product was 13 mg, and its estimated purity by LCMS analysis was 98.3%. Analysis condition 1: Retention time=1.87 min; ESI-MS(+) m/z (M+3H)³⁺: 1031.4.

### Preparation of Example 5142

Example 5142 was prepared on a 50 μmol scale. The yield of the product was 5 mg, and its estimated purity by LCMS analysis was 94.500. Analysis condition 1: Retention time=1.56 min; ESI-MS(+) m/z (M+3H)³⁺: 1141.2.

### Preparation of Example 5143

Example 5143 was prepared on a 50 μmol scale. The yield of the product was 21.5 mg, and its estimated purity by LCMS analysis was 96%. Analysis condition 2: Retention time=1.52 min; ESI-MS(+) m/z (M+3H)³⁺: 1101.

### Preparation of Example 5144

Example 5144 was prepared on a 50 μmol scale. The yield of the product was 2.9 mg, and its estimated purity by LCMS analysis was 100%. Analysis condition 2: Retention time=1.53 min: ESI-MS(+) m/z (M+3H)³⁺: 1141.5.

### Preparation of Example 5145

Example 5145 was prepared on a 50 μmol scale. The yield of the product was 5.2 mg, and its estimated purity by LCMS analysis was 100%. Analysis condition 2: Retention time=1.52 min; ESI-MS(+) m/z (M+3H)³⁺: 1184.

### Preparation of Example 5146

Example 5146 was prepared on a 50 μmol scale. The yield of the product was 8.8 mg, and its estimated purity by LCMS analysis was 86%. Analysis condition 1: Retention time=1.69 min; ESI-MS(+) m/z (M+3H)³⁺: 1126.1.

### Preparation of Example 5147

Example 5147 was prepared on a 50 μmol scale. The yield of the product was 22.4 mg, and its estimated purity by LCMS analysis was 96.5%. Analysis condition 1: Retention time=1.63 min; ESI-MS(+) m/z (M+3H)³⁺: 1086.

### Preparation of Example 5148

Example 5148 was prepared on a 50 μmol scale. The yield of the product was 4.5 mg, and its estimated purity by LCMS analysis was 82.8%. Analysis condition 1: Retention time=1.73 min; ESI-MS(+) m/z (M+3H)³⁺: 1142.9.

### Preparation of Example 5149

Example 5149 was prepared on a 50 μmol scale. The yield of the product was 14.7 mg, and its estimated purity by LCMS analysis was 100%. Analysis condition 2: Retention time=1.5 min; ESI-MS(+) m/z (M+3H)³⁺: 1157.1.

### Preparation of Example 5150

Example 5150 was prepared on a 50 μmol scale. The yield of the product was 3.7 mg, and its estimated purity by LCMS analysis was 9700. Analysis condition 1: Retention time=1.67 min; ESI-MS(+) m/z (M+3H)³⁺: 1955.2.

### Preparation of Example 5151

Example 5151 was prepared on a 50 μmol scale. The yield of the product was 7.4 mg, and its estimated purity by LCMS analysis was 95.2%. Analysis condition 1: Retention time=1.59 min; ESI-MS(+) m/z (M+3H)³⁺: 1098.1.

### Preparation of Example 5152

Example 5152 was prepared on a 50 μmol scale. The yield of the product was 5.8 mg, and its estimated purity by LCMS analysis was 86.6%. Analysis condition 2: Retention time=1.66 min; ESI-MS(+) m/z (M+3H)³⁺: 1156.2.

### Preparation of Example 5153

Example 5153 was prepared on a 50 μmol scale. The yield of the product was 18.8 mg, and its estimated purity by LCMS analysis was 84.2%. Analysis condition 2: Retention time=1.59 min; ESI-MS(+) m/z (M+3H)³⁺: 1189.1.

### Preparation of Example 5154

Example 5154 was prepared on a 50 μmol scale. The yield of the product was 17.2 mg, and its estimated purity by LCMS analysis was 98.8%. Analysis condition 2: Retention time=1.65 min; ESI-MS(+) m/z (M+3H)³⁺: 1128.9.

### Preparation of Example 5155

Example 5155 was prepared on a 50 μmol scale. The yield of the product was 15.7 mg, and its estimated purity by LCMS analysis was 88.2%. Analysis condition 2: Retention time=1.54 min: ESI-MS(+) m/z (M+3H)³⁺: 1172.

### Preparation of Example 5156

Example 5156 was prepared on a 50 μmol scale. The yield of the product was 27.4 mg, and its estimated purity by LCMS analysis was 77.6%. Analysis condition 1: Retention time=1.78 min; ESI-MS(+) m/z (M+3H)³⁺: 1204.4.

### Preparation of Example 5157

Example 5157 was prepared on a 50 μmol scale. The yield of the product was 25.5 mg, and its estimated purity by LCMS analysis was 91.9%. Analysis condition 2: Retention time=1.69 min; ESI-MS(+) m/z (M+3H)³⁺: 1156.1.

### Preparation of Example 5158

Example 5158 was prepared on a 50 μmol scale. The yield of the product was 22.3 mg, and its estimated purity by LCMS analysis was 90.6%. Analysis condition 1: Retention time=1.76 min; ESI-MS(+) m/z (M+3H)³⁺: 1188.5.

### Preparation of Example 5159

Example 5159 was prepared on a 50 μmol scale. The yield of the product was 26.6 mg, and its estimated purity by LCMS analysis was 100%. Analysis condition 1: Retention time=1.75 min; ESI-MS(+) m/z (M+3H)³⁺: 1156.7.

### Preparation of Example 5160

Example 5160 was prepared on a 50 μmol scale. The yield of the product was 19.9 mg, and its estimated purity by LCMS analysis was 91.9%. Analysis condition 2: Retention time=1.63 min; ESI-MS(+) m/z (M+3H)³⁺: 1128.5.

### Preparation of Example 5161

Example 5161 was prepared on a 50 μmol scale. The yield of the product was 25.4 mg, and its estimated purity by LCMS analysis was 90.3%. Analysis condition 1: Retention time=1.68 min; ESI-MS(+) m/z (M+3H)³⁺: 1171.7.

### Preparation of Example 5162

Example 5162 was prepared on a 50 μmol scale. The yield of the product was 27.6 mg, and its estimated purity by LCMS analysis was 100%. Analysis condition 1: Retention time=1.74 min; ESI-MS(+) m/z (M+3H)³⁺: 1143.7.

### Preparation of Example 5163

Example 5163 was prepared on a 50 μmol scale. The yield of the product was 49.7 mg, and its estimated purity by LCMS analysis was 87.7%. Analysis condition 1: Retention time=1.77 min; ESI-MS(+) m/z (M+3H)³⁺: 1204.2.

### Preparation of Example 5164

Example 5164 was prepared on a 50 μmol scale. The yield of the product was 13.9 mg, and its estimated purity by LCMS analysis was 100%. Analysis condition 2: Retention time=1.47 min; ESI-MS(+) m/z (M+3H)³⁺: 1182.1.

### Preparation of Example 5165

Example 5165 was prepared on a 50 μmol scale. The yield of the product was 13.2 mg, and its estimated purity by LCMS analysis was 92%. Analysis condition 2: Retention time=1.74 min; ESI-MS(+) m/z (M+3H)³⁺: 1109.6.

### Preparation of Example 5166

Example 5166 was prepared on a 50 μmol scale. The yield of the product was 8.5 mg, and its estimated purity by LCMS analysis was 83.7%. Analysis condition 1: Retention time=1.81 min; ESI-MS(+) m/z (M+3H)³⁺: 1089.6.

### Preparation of Example 5167

Example 5167 was prepared on a 50 μmol scale. The yield of the product was 7.8 mg, and its estimated purity by LCMS analysis was 97.2%. Analysis condition 2: Retention time=1.54 min: ESI-MS(+) m/z (M+3H)³⁺: 1144.

### Preparation of Example 5168

Example 5168 was prepared on a 50 μmol scale. The yield of the product was 1.3 mg, and its estimated purity by LCMS analysis was 85.3%. Analysis condition 2: Retention time=1.48 min; ESI-MS(+) m/z (M+3H)³⁺: 1093.6.

### Preparation of Example 5169

Example 5169 was prepared on a 50 μmol scale. The yield of the product was 3.4 mg, and its estimated purity by LCMS analysis was 92.4%. Analysis condition 1: Retention time=1.63 min; ESI-MS(+) m/z (M+3H)³⁺: 1069.6.

### Preparation of Example 5170

Example 5170 was prepared on a 50 μmol scale. The yield of the product was 1.7 mg, and its estimated purity by LCMS analysis was 86%. Analysis condition 1: Retention time=1.64 min; ESI-MS(+) m/z (M+3H)³⁺: 1048.

### Preparation of Example 5171

Example 5171 was prepared on a 25 μmol scale. The yield of the product was 2.7 mg, and its estimated purity by LCMS analysis was 82%. Analysis condition 2: Retention time=1.43 min; ESI-MS(+) m/z (M+3H)³⁺: 1105.4.

### Preparation of Example 5172

Example 5172 was prepared on a 25 μmol scale. The yield of the product was 6.2 mg, and its estimated purity by LCMS analysis was 85.2%. Analysis condition 2: Retention time=1.52 min; ESI-MS(+) m/z (M+3H)³⁺: 1057.4.

### Preparation of Example 5173

Example 5173 was prepared on a 25 μmol scale. The yield of the product was 3.7 mg, and its estimated purity by LCMS analysis was 9400. Analysis condition 2: Retention time=1.59 min: ESI-MS(+) m/z (M+3H)³⁺: 1123.4.

### Preparation of Example 5174

Example 5174 was prepared on a 25 μmol scale. The yield of the product was 6.9 mg, and its estimated purity by LCMS analysis was 91.2%. Analysis condition 2: Retention time=1.61 min; ESI-MS(+) m/z (M+3H)³⁺: 1059.9.

### Preparation of Example 5175

Example 5175 was prepared on a 25 μmol scale. The yield of the product was 2.8 mg, and its estimated purity by LCMS analysis was 91.5%. Analysis condition 2: Retention time=1.52 min; ESI-MS(+) m/z (M+3H)³⁺: 1039.9.

### Preparation of Example 5176

Example 5176 was prepared on a 50 μmol scale. The yield of the product was 18 mg, and its estimated purity by LCMS analysis was 87.6%. Analysis condition 1: Retention time=1.6 min: ESI-MS(+) m/z (M+3H)³⁺: 1071.5.

### Preparation of Example 5177

Example 5177 was prepared on a 25 μmol scale. The yield of the product was 0.8 mg, and its estimated purity by LCMS analysis was 94.2%. Analysis condition 1: Retention time=1.63 min; ESI-MS(+) m/z (M+3H)³⁺: 1116.9.

### Preparation of Example 5178

Example 5178 was prepared on a 25 μmol scale. The yield of the product was 3.6 mg, and its estimated purity by LCMS analysis was 81.2%. Analysis condition 2: Retention time=1.48 min; ESI-MS(+) m/z (M+3H)³⁺: 1026.3.

### Preparation of Example 5179

Example 5179 was prepared on a 25 μmol scale. The yield of the product was 1.5 mg, and its estimated purity by LCMS analysis was 92.5%. Analysis condition 2: Retention time=1.52 min; ESI-MS(+) m/z (M+3H)³⁺: 1033.5.

### Preparation of Example 5180

Example 5180 was prepared on a 25 μmol scale. The yield of the product was 2 mg, and its estimated purity by LCMS analysis was 95.7%. Analysis condition 2: Retention time=1.53 min; ESI-MS(+) m/z (M+3H)³⁺: 1040.3.

### Preparation of Example 5181

Example 5181 was prepared on a 25 μmol scale. The yield of the product was 1.1 mg, and its estimated purity by LCMS analysis was 100%. Analysis condition 2: Retention time=1.49 min; ESI-MS(+) m/z (M+3H)³⁺: 1047.3.

### Preparation of Example 5182

Example 5182 was prepared on a 25 μmol scale. The yield of the product was 1.8 mg, and its estimated purity by LCMS analysis was 85.2%. Analysis condition 2: Retention time=1.5 min; ESI-MS(+) m/z (M+3H)³⁺: 703.3.

### Preparation of Example 5183

Example 5183 was prepared on a 25 μmol scale. The yield of the product was 5.2 mg, and its estimated purity by LCMS analysis was 88.8%. Analysis condition 1: Retention time=1.5 min; ESI-MS(+) m/z (M+3H)³⁺: 1061.3.

### Preparation of Example 5184

Example 5184 was prepared on a 25 μmol scale. The yield of the product was 3 mg, and its estimated purity by LCMS analysis was 85.3%. Analysis condition 2: Retention time=1.48 min; ESI-MS(+) m/z (M+3H)³⁺: 1068.4.

### Preparation of Example 5185

Example 5185 was prepared on a 25 μmol scale. The yield of the product was 7 mg, and its estimated purity by LCMS analysis was 90.8%. Analysis condition 2: Retention time=1.54 min; ESI-MS(+) m/z (M+3H)³⁺: 1039.

### Preparation of Example 5186

Example 5186 was prepared on a 25 μmol scale. The yield of the product was 6.5 mg, and its estimated purity by LCMS analysis was 94.5%. Analysis condition 2: Retention time=1.67 min; ESI-MS(+) m/z (M+3H)³⁺: 1066.8.

### Preparation of Example 5187

Example 5187 was prepared on a 25 μmol scale. The yield of the product was 3.1 mg, and its estimated purity by LCMS analysis was 85.3%. Analysis condition 2: Retention time=1.46 min; ESI-MS(+) m/z (M+3H)³⁺: 1040.3.

### Preparation of Example 5188

Example 5188 was prepared on a 50 μmol scale. The yield of the product was 13.3 mg, and its estimated purity by LCMS analysis was 89%. Analysis condition 1: Retention time=1.58 min; ESI-MS(+) m/z (M+3H)³⁺: 1071.5.

### Preparation of Example 5189

Example 5189 was prepared on a 50 μmol scale. The yield of the product was 22.1 mg, and its estimated purity by LCMS analysis was 89.2%. Analysis condition 2: Retention time=1.47 min; ESI-MS(+) m/z (M+3H)³⁺: 1064.6.

### Preparation of Example 5190

Example 5190 was prepared on a 50 μmol scale. The yield of the product was 16.8 mg, and its estimated purity by LCMS analysis was 86.4%. Analysis condition 2: Retention time=1.51 min; ESI-MS(+) m/z (M+3H)³⁺: 1064.6.

### Preparation of Example 5191

Example 5191 was prepared on a 50 μmol scale. The yield of the product was 3.5 mg, and its estimated purity by LCMS analysis was 96%. Analysis condition 2: Retention time=1.56 min: ESI-MS(+) m/z (M+3H)³⁺: 708.

### Preparation of Example 5192

Example 5192 was prepared on a 50 μmol scale. The yield of the product was 3.9 mg, and its estimated purity by LCMS analysis was 92.7%. Analysis condition 2: Retention time=1.51 min; ESI-MS(+) m/z (M+3H)³⁺: 1039.3.

### Preparation of Example 5193

Example 5193 was prepared on a 50 μmol scale. The yield of the product was 4.5 mg, and its estimated purity by LCMS analysis was 80.8%. Analysis condition 1: Retention time=1.81 min; ESI-MS(+) m/z (M+3H)³⁺: 1067.1.

### Preparation of Example 5194

Example 5194 was prepared on a 50 μmol scale. The yield of the product was 7.2 mg, and its estimated purity by LCMS analysis was 90%. Analysis condition 1: Retention time=1.53 min; ESI-MS(+) m/z (M+3H)³⁺: 1057.7.

### Preparation of Example 5195

Example 5195 was prepared on a 50 μmol scale. The yield of the product was 29.4 mg, and its estimated purity by LCMS analysis was 86.4%. Analysis condition 1: Retention time=1.91 min; ESI-MS(+) m/z (M+3H)³⁺: 1110.5.

### Preparation of Example 5196

Example 5196 was prepared on a 50 μmol scale. The yield of the product was 14.1 mg, and its estimated purity by LCMS analysis was 94.7%. Analysis condition 2: Retention time=1.48 min; ESI-MS(+) m/z (M+3H)³⁺: 1084.1.

### Preparation of Example 5197

Example 5197 was prepared on a 50 μmol scale. The yield of the product was 10 mg, and its estimated purity by LCMS analysis was 72.300. Analysis condition 1: Retention time=1.68 min; ESI-MS(+) m/z (M+3H)³⁺: 1122.7.

### Preparation of Example 5198

Example 5198 was prepared on a 50 μmol scale. The yield of the product was 19.4 mg, and its estimated purity by LCMS analysis was 90%. Analysis condition 1: Retention time=1.91 min; ESI-MS(+) m/z (M+3H)³⁺: 1121.6.

### Preparation of Example 5199

Example 5199 was prepared on a 50 μmol scale. The yield of the product was 12 mg, and its estimated purity by LCMS analysis was 83.3%. Analysis condition 1: Retention time=1.91 min; ESI-MS(+) m/z (M+3H)³⁺: 1128.6.

### Preparation of Example 5200

Example 5200 was prepared on a 50 μmol scale. The yield of the product was 31.7 mg, and its estimated purity by LCMS analysis was 89.1%. Analysis condition 2: Retention time=1.68 min; ESI-MS(+) m/z (M+3H)³⁺: 1127.6.

### Preparation of Example 5201

Example 5201 was prepared on a 50 μmol scale. The yield of the product was 32.9 mg, and its estimated purity by LCMS analysis was 86.8%. Analysis condition 2: Retention time=1.53 min; ESI-MS(+) m/z (M+3H)³⁺: 1098.5.

### Preparation of Example 5202

Example 5202 was prepared on a 50 μmol scale. The yield of the product was 19.6 mg, and its estimated purity by LCMS analysis was 100%. Analysis condition 2: Retention time=1.53 min; ESI-MS(+) m/z (M+3H)³⁺: 1143.4.

### Preparation of Example 5203

Example 5203 was prepared on a 50 μmol scale. The yield of the product was 19 mg, and its estimated purity by LCMS analysis was 87.7%. Analysis condition 1: Retention time=1.89 min; ESI-MS(+) m/z (M+3H)³⁺: 1096.6.

### Preparation of Example 5204

Example 5204 was prepared on a 50 μmol scale. The yield of the product was 20.8 mg, and its estimated purity by LCMS analysis was 96.9%. Analysis condition 1. Retention time=1.76 min: ESI-MS(+) m/z (M+3H)³⁺: 1097.2.

### Preparation of Example 5205

Example 5205 was prepared on a 50 μmol scale. The yield of the product was 19.3 mg, and its estimated purity by LCMS analysis was 84.9%. Analysis condition 1: Retention time=1.58 min; ESI-MS(+) m/z (M+3H)³⁺: 1033.4.

### Preparation of Example 5206

Example 5206 was prepared on a 50 μmol scale. The yield of the product was 25.1 mg, and its estimated purity by LCMS analysis was 98.9%. Analysis condition 2: Retention time=1.87 min; ESI-MS(+) m/z (M+3H)³⁺: 1076.4.

### Preparation of Example 5207

Example 5207 was prepared on a 50 μmol scale. The yield of the product was 6.9 mg, and its estimated purity by LCMS analysis was 95.8%. Analysis condition 2: Retention time=1.75 min; ESI-MS (+) m/z (M+3H)³⁺: 1130.8.

### Preparation of Example 5208

Example 5208 was prepared on a 50 μmol scale. The yield of the product was 21.6 mg, and its estimated purity by LCMS analysis was 98.3%. Analysis condition 1: Retention time=1.77 min; ESI-MS(+) m/z (M+3H)³⁺: 1070.6.

### Preparation of Example 5209

Example 5209 was prepared on a 50 μmol scale. The yield of the product was 7 mg, and its estimated purity by LCMS analysis was 99.1%. Analysis condition 1: Retention time=1.87 min; ESI-MS(+) m/z (M+3H)³⁺: 1133.

### Preparation of Example 5210

Example 5210 was prepared on a 50 μmol scale. The yield of the product was 5.6 mg, and its estimated purity by LCMS analysis was 9700. Analysis condition 2: Retention time=1.57 min; ESI-MS(+) m/z (M+3H)³⁺: 1126.2.

### Preparation of Example 5211

Example 5211 was prepared on a 50 μmol scale. The yield of the product was 26.4 mg, and its estimated purity by LCMS analysis was 81.8%. Analysis condition 2: Retention time=1.7 min; ESI-MS(+) m/z (M+3H)³⁺: 1080.9.

### Preparation of Example 5212

Example 5212 was prepared on a 50 μmol scale. The yield of the product was 16.3 mg, and its estimated purity by LCMS analysis was 97.3%. Analysis condition 2: Retention time=1.69 min; ESI-MS(+) m/z (M+3H)³⁺: 1054.2.

### Preparation of Example 5213

Example 5213 was prepared on a 50 μmol scale. The yield of the product was 16.6 mg, and its estimated purity by LCMS analysis was 91%. Analysis condition 1: Retention time=1.97 min: ESI-MS(+) m/z (M+3H)³⁺: 1079.3.

### Preparation of Example 5214

Example 5214 was prepared on a 50 μmol scale. The yield of the product was 15.9 mg, and its estimated purity by LCMS analysis was 85.2%. Analysis condition 2: Retention time=1.82 min; ESI-MS(+) m/z (M+3H)³⁺: 1052.3.

### Preparation of Example 5215

Example 5215 was prepared on a 50 μmol scale. The yield of the product was 21.5 mg, and its estimated purity by LCMS analysis was 92.9%. Analysis condition 1: Retention time=1.74 min; ESI-MS(+) m/z (M+3H)³⁺: 1052.8.

### Preparation of Example 5216

Example 5216 was prepared on a 50 μmol scale. The yield of the product was 21.7 mg, and its estimated purity by LCMS analysis was 89.3%. Analysis condition 1: Retention time=1.68 min; ESI-MS(+) m/z (M+3H)³⁺: 1073.4.

### Preparation of Example 5217

Example 5217 was prepared on a 50 μmol scale. The yield of the product was 34.2 mg, and its estimated purity by LCMS analysis was 89.6%. Analysis condition 2: Retention time=1.73 min: ESI-MS(+) m/z (M+3H)³⁺: 1046.6.

### Preparation of Example 5218

Example 5218 was prepared on a 50 μmol scale. The yield of the product was 42.7 mg, and its estimated purity by LCMS analysis was 85.8%. Analysis condition 2: Retention time=1.93 min; ESI-MS(+) m/z (M+3H)³⁺: 1045.1.

### Preparation of Example 5219

Example 5219 was prepared on a 50 μmol scale. The yield of the product was 38.4 mg, and its estimated purity by LCMS analysis was 86.8%. Analysis condition 2: Retention time=1.76 min; ESI-MS(+) m/z (M+3H)³⁺: 1045.3.

### Preparation of Example 5220

Example 5220 was prepared on a 50 μmol scale. The yield of the product was 27.4 mg, and its estimated purity by LCMS analysis was 82.3%. Analysis condition 2: Retention time=1.56 min; ESI-MS(+) m/z (M+3H)³⁺: 1075.3.

### Preparation of Example 5221

Example 5221 was prepared on a 50 μmol scale. The yield of the product was 30.6 mg, and its estimated purity by LCMS analysis was 90.1%. Analysis condition 2: Retention time=1.68 min: ESI-MS(+) m/z (M+3H)³⁺: 1053.6.

### Preparation of Example 5222

Example 5222 was prepared on a 50 μmol scale. The yield of the product was 45.4 mg, and its estimated purity by LCMS analysis was 91.8%. Analysis condition 2: Retention time=1.88 min; ESI-MS(+) m/z (M+3H)³⁺: 1052.

### Preparation of Example 5223

Example 5223 was prepared on a 50 μmol scale. The yield of the product was 46.9 mg, and its estimated purity by LCMS analysis was 80.5%. Analysis condition 2: Retention time=1.75 min; ESI-MS(+) m/z (M+3H)³⁺: 1052.4.

### Preparation of Example 5224

Example 5224 was prepared on a 50 μmol scale. The yield of the product was 27.1 mg, and its estimated purity by LCMS analysis was 89.2%. Analysis condition 2: Retention time=1.44 min; ESI-MS(+) m/z (M+3H)³⁺: 1083.4.

### Preparation of Example 5225

Example 5225 was prepared on a 50 μmol scale. The yield of the product was 30.1 mg, and its estimated purity by LCMS analysis was 83.9%. Analysis condition 2: Retention time=1.71 min; ESI-MS(+) m/z (M+3H)³⁺: 1040.9.

### Preparation of Example 5226

Example 5226 was prepared on a 50 μmol scale. The yield of the product was 39.1 mg, and its estimated purity by LCMS analysis was 83.4%. Analysis condition 1: Retention time=1.97 min; ESI-MS(+) m/z (M+3H)³⁺: 1066.4.

### Preparation of Example 5227

Example 5227 was prepared on a 50 μmol scale. The yield of the product was 31.2 mg, and its estimated purity by LCMS analysis was 92.5%. Analysis condition 2: Retention time=1.82 min; ESI-MS(+) m/z (M+3H)³⁺: 1040.

### Preparation of Example 5228

Example 5228 was prepared on a 50 μmol scale. The yield of the product was 20.2 mg, and its estimated purity by LCMS analysis was 97.2%. Analysis condition 2: Retention time=1.54 min; ESI-MS(+) m/z (M+3H)³⁺: 1139.7.

### Preparation of Example 5229

Example 5229 was prepared on a 50 μmol scale. The yield of the product was 16.1 mg, and its estimated purity by LCMS analysis was 95.7%. Analysis condition 1: Retention time=1.93 min: ESI-MS(+) m/z (M+3H)³⁺: 1147.9.

### Preparation of Example 5230

Example 5230 was prepared on a 50 μmol scale. The yield of the product was 9.2 mg, and its estimated purity by LCMS analysis was 96.6%. Analysis condition 2: Retention time=1.47 min; ESI-MS(+) m/z (M+3H)³⁺: 1127.9.

### Preparation of Example 5231

Example 5231 was prepared on a 50 μmol scale. The yield of the product was 8.4 mg, and its estimated purity by LCMS analysis was 950%. Analysis condition 2: Retention time=1.63 min; ESI-MS(+) m/z (M+3H)³⁺: 1124.2.

### Preparation of Example 5232

Example 5232 was prepared on a 50 μmol scale. The yield of the product was 3.4 mg, and its estimated purity by LCMS analysis was 96.7%. Analysis condition 1: Retention time=1.88 min; ESI-MS(+) m/z (M+3H)³⁺: 1131.

### Preparation of Example 5233

Example 5233 was prepared on a 50 μmol scale. The yield of the product was 4 mg, and its estimated purity by LCMS analysis was 100%. Analysis condition 1: Retention time=1.77 min; ESI-MS(+) m/z (M+3H)³⁺: 1127.8.

### Preparation of Example 5234

Example 5234 was prepared on a 50 μmol scale. The yield of the product was 5.3 mg, and its estimated purity by LCMS analysis was 98%. Analysis condition 2: Retention time=1.41 min; ESI-MS(+) m/z (M+3H)³⁺: 1148.1.

### Preparation of Example 5235

Example 5235 was prepared on a 50 μmol scale. The yield of the product was 27.9 mg, and its estimated purity by LCMS analysis was 97.9%. Analysis condition 1: Retention time=1.64 min; ESI-MS(+) m/z (M+3H)³⁺: 1044.7.

### Preparation of Example 5236

Example 5236 was prepared on a 50 μmol scale. The yield of the product was 32.4 mg, and its estimated purity by LCMS analysis was 93.2%. Analysis condition 2: Retention time=1.81 min; ESI-MS(+) m/z (M+3H)³⁺: 1016.2.

### Preparation of Example 5237

Example 5237 was prepared on a 50 μmol scale. The yield of the product was 20.9 mg, and its estimated purity by LCMS analysis was 90.5%. Analysis condition 2: Retention time=1.67 min; ESI-MS(+) m/z (M+3H)³⁺: 1074.7.

### Preparation of Example 5238

Example 5238 was prepared on a 50 μmol scale. The yield of the product was 20.9 mg, and its estimated purity by LCMS analysis was 9500. Analysis condition 1: Retention time=1.82 min; ESI-MS(+) m/z (M+3H)³⁺: 1072.7.

### Preparation of Example 5239

Example 5239 was prepared on a 50 μmol scale. The yield of the product was 27.3 mg, and its estimated purity by LCMS analysis was 85.1%. Analysis condition 2: Retention time=1.68 min; ESI-MS(+) m/z (M+3H)³⁺: 1046.4.

### Preparation of Example 5240

Example 5240 was prepared on a 50 μmol scale. The yield of the product was 39.2 mg, and its estimated purity by LCMS analysis was 89.4%. Analysis condition 2: Retention time=1.91 min; ESI-MS(+) m/z (M+3H)³⁺: 1051.4.

### Preparation of Example 5241

Example 5241 was prepared on a 50 μmol scale. The yield of the product was 47.1 mg, and its estimated purity by LCMS analysis was 93.9%. Analysis condition 1: Retention time=2.02 min; ESI-MS(+) m/z (M+3H)³⁺: 1049.9.

### Preparation of Example 5242

Example 5242 was prepared on a 50 μmol scale. The yield of the product was 50.2 mg, and its estimated purity by LCMS analysis was 96.4%. Analysis condition 2: Retention time=1.86 min; ESI-MS(+) m/z (M+3H)³⁺: 1023.4.

### Preparation of Example 5243

Example 5243 was prepared on a 50 μmol scale. The yield of the product was 24.3 mg, and its estimated purity by LCMS analysis was 100%. Analysis condition 2: Retention time=1.99 min; ESI-MS(+) m/z (M+3H)³⁺: 1036.8.

### Preparation of Example 5244

Example 5244 was prepared on a 50 μmol scale. The yield of the product was 19.6 mg, and its estimated purity by LCMS analysis was 86.6%. Analysis condition 2: Retention time=1.79 min; ESI-MS(+) m/z (M+3H)³⁺: 1008.6.

### Preparation of Example 5245

Example 5245 was prepared on a 50 μmol scale. The yield of the product was 10.6 mg, and its estimated purity by LCMS analysis was 91.7%. Analysis condition 2: Retention time=1.75 min; ESI-MS(+) m/z (M+3H)³⁺: 1026.8.

### Preparation of Example 5246

Example 5246 was prepared on a 50 μmol scale. The yield of the product was 16.5 mg, and its estimated purity by LCMS analysis was 91.8%. Analysis condition 2: Retention time=2.21 min; ESI-MS(+) m/z (M+3H)³⁺: 1025.3.

### Preparation of Example 5247

Example 5247 was prepared on a 50 μmol scale. The yield of the product was 15.8 mg, and its estimated purity by LCMS analysis was 93.4%. Analysis condition 1: Retention time=1.66 min ESI-MS(+) m/z (M+3H)³⁺: 1995.8.

### Preparation of Example 5248

Example 5248 was prepared on a 50 μmol scale. The yield of the product was 20.5 mg, and its estimated purity by LCMS analysis was 93.5%. Analysis condition 2: Retention time=1.75 min; ESI-MS(+) m/z (M+3H)³⁺: 1055.1.

### Preparation of Example 5249

Example 5249 was prepared on a 50 μmol scale. The yield of the product was 9.5 mg, and its estimated purity by LCMS analysis was 100%. Analysis condition 2: Retention time=1.54 min: ESI-MS(+) m/z (M+3H)³⁺: 1029.1.

### Preparation of Example 5250

Example 5250 was prepared on a 50 μmol scale. The yield of the product was 23.4 mg, and its estimated purity by LCMS analysis was 94.200. Analysis condition 2: Retention time=1.56 min; ESI-MS(+) m/z (M+3H)³⁺: 1120.4.

### Preparation of Example 5251

Example 5251 was prepared on a 50 μmol scale. The yield of the product was 7.6 mg, and its estimated purity by LCMS analysis was 93.8%. Analysis condition 1: Retention time=1.65 min; ESI-MS(+) m/z (M+3H)³⁺: 1077.8.

### Preparation of Example 5252

Example 5252 was prepared on a 50 μmol scale. The yield of the product was 20 mg, and its estimated purity by LCMS analysis was 93.1%. Analysis condition 2: Retention time=1.76 min; ESI-MS(+) m/z (M+3H)³⁺: 1091.2.

### Preparation of Example 5253

Example 5253 was prepared on a μmol scale. The yield of the product was 11.6 mg, and its estimated purity by LCMS analysis was 95.1%. Analysis condition 1: Retention time=1.76 min; ESI-MS(+) m/z (M+3H)³⁺: 1186.2.

### Preparation of Example 5254

Example 5254 was prepared on a μmol scale. The yield of the product was 27.3 mg, and its estimated purity by LCMS analysis was 92.6%. Analysis condition 1: Retention time=1.69 min: ESI-MS(+) m/z (M+3H)³⁺: 11037.8.

Example 5255 was prepared on a mol scale. The yield of the product was 27.8 mg, and its estimated purity by LCMS analysis was 92.62%. Analysis condition 2: Retention time=1.48 min; ESI-MS(+) m/z (M+3H)³⁺: 11037.2.

### Preparation of Example 5256

Example 5256 was prepared on a μmol scale. The yield of the product was 22.8 mg, and its estimated purity by LCMS analysis was 90.2%. Analysis condition 2: Retention time=1.44 min; ESI-MS(+) m/z (M+3H)³⁺: 1092.2.

### Preparation of Example 5257

Example 5257 was prepared on a μmol scale. The yield of the product was 22.8 mg, and its estimated purity by LCMS analysis was 92.300. Analysis condition 1: Retention time=1.7 min: ESI-MS(+) m/z (M+3H)³⁺: 1136.3.

### Preparation of Example 5258

Example 5258 was prepared on a μmol scale. The yield of the product was 35.7 mg, and its estimated purity by LCMS analysis was 91.1%. Analysis condition 1: Retention time=1.64 min; ESI-MS(+) m/z (M+3H)³⁺: 1092.

### Preparation of Example 5259

Example 5259 was prepared on a μmol scale. The yield of the product was 22.5 mg, and its estimated purity by LCMS analysis was 92.2%. Analysis condition 1: Retention time=1.72 min: ESI-MS(+H) m/z (M+3H)³⁺: 1085.

### Preparation of Example 5260

Example 5260 was prepared on a μmol scale. The yield of the product was 47.6 mg, and its estimated purity by LCMS analysis was 90.2%. Analysis condition 2: Retention time=1.5 min; ESI-MS(+) m/z (M+3H)³⁺: 1085.

### Preparation of Example 5261

Example 5261 was prepared on a 50 mol scale. The yield of the product was 97.4 mg, and its estimated purity by LCMS analysis was 94.2%. Analysis condition 2: Retention time=1.55 min: ESI-MS(+) m/z (M+3H)³⁺: 1056.2.

### Preparation of Example 5262

Example 5262 was prepared on a 50 μmol scale. The yield of the product was 40.1 mg, and its estimated purity by LCMS analysis was 95.5%. Analysis condition 1: Retention time=1.57 min; ESI-MS(+) m/z (M+3H)³⁺: 1105.7.

### Preparation of Example 5263

Example 5263 was prepared on a 50 μmol scale. The yield of the product was 41.1 mg, and its estimated purity by LCMS analysis was 95.4%. Analysis condition 1: Retention time=1.63 min; ESI-MS(+) m/z (M+3H)³⁺: 1081.6.

### Preparation of Example 5264

Example 5264 was prepared on a 50 μmol scale. The yield of the product was 30.9 mg, and its estimated purity by LCMS analysis was 95.3%. Analysis condition 1: Retention time=1.65 min; ESI-MS(+) m/z (M+3H)³⁺: 1073.7.

### Preparation of Example 5265

Example 5265 was prepared on a 50 μmol scale. The yield of the product was 29.6 mg, and its estimated purity by LCMS analysis was 85%. Analysis condition 2: Retention time=1.66 min; ESI-MS(+) m/z (M+3H)³⁺: 1064.1.

### Preparation of Example 5266

Example 5266 was prepared on a 50 μmol scale. The yield of the product was 13.1 mg, and its estimated purity by LCMS analysis was 87.3%. Analysis condition 2: Retention time=1.56 min; ESI-MS(+) m/z (M+3H)³⁺: 1018.9.

### Preparation of Example 5267

Example 5267 was prepared on a 50 μmol scale. The yield of the product was 39.2 mg, and its estimated purity by LCMS analysis was 89.8%. Analysis condition 2: Retention time=1.63 min; ESI-MS(+) m/z (M+3H)³⁺: 1057.2.

### Preparation of Example 5268

Example 5268 was prepared on a 50 μmol scale. The yield of the product was 19 mg, and its estimated purity by LCMS analysis was 85.4%. Analysis condition 1: Retention time=1.69 min; ESI-MS(+) m/z (M+3H)³⁺: 1040.

### Preparation of Example 5269

Example 5269 was prepared on a 50 μmol scale. The yield of the product was 14.9 mg, and its estimated purity by LCMS analysis was 94.6%. Analysis condition 2: Retention time=1.6 min; ESI-MS(+) m/z (M+3H)³⁺: 1040.

### Preparation of Example 5270

Example 5270 was prepared on a 50 μmol scale. The yield of the product was 22.7 mg, and its estimated purity by LCMS analysis was 85%. Analysis condition 2: Retention time=1.52 min ESI-MS(+) m/z (M+3H)³⁺: 1057.

### Preparation of Example 5271

Example 5271 was prepared on a 50 μmol scale. The yield of the product was 25.8 mg, and its estimated purity by LCMS analysis was 84.8%. Analysis condition 2: Retention time=1.56 min; ESI-MS(+) m/z (M+3H)³⁺: 1065.1.

### Preparation of Example 5272

Example 5272 was prepared on a 50 μmol scale. The yield of the product was 10.6 mg, and its estimated purity by LCMS analysis was 80.3%. Analysis condition 2: Retention time=1.61 min; ESI-MS(+) m/z (M+3H)³⁺: 1039.9.

### Preparation of Example 5273

Example 5273 was prepared on a 50 μmol scale. The yield of the product was 15.4 mg, and its estimated purity by LCMS analysis was 98.6%. Analysis condition 1: Retention time=1.69 min: ESI-MS(+) m/z (M+3H)³⁺: 1064.

### Preparation of Example 5274

Example 5274 was prepared on a 50 μmol scale. The yield of the product was 10.6 mg, and its estimated purity by LCMS analysis was 99%. Analysis condition 1: Retention time=1.56 min; ESI-MS(+) m/z (M+3H)³⁺: 1052.1.

### Preparation of Example 5275

Example 5275 was prepared on a 50 μmol scale. The yield of the product was 2 mg, and its estimated purity by LCMS analysis was 98.3%. Analysis condition 1: Retention time=1.6 min; ESI-MS(+) m/z (M+3H)³⁺: 1041.

### Preparation of Example 5276

Example 5276 was prepared on a 50 μmol scale. The yield of the product was 19.6 mg, and its estimated purity by LCMS analysis was 88.2%. Analysis condition 1: Retention time=1.69 min; ESI-MS(+) m/z (M+3H)³⁺: 1091.1.

### Preparation of Example 5277

Example 5277 was prepared on a 50 μmol scale. The yield of the product was 33.8 mg, and its estimated purity by LCMS analysis was 94.5%. Analysis condition 1: Retention time=1.88 min; ESI-MS(+) m/z (M+3H)³⁺: 1064.5.

### Preparation of Example 5278

Example 5278 was prepared on a 50 μmol scale. The yield of the product was 17.1 mg, and its estimated purity by LCMS analysis was 100%. Analysis condition 1: Retention time=1.51 min; ESI-MS(+) m/z (M+3H)³⁺: 1092.8.

### Preparation of Example 5279

Example 5279 was prepared on a 50 μmol scale. The yield of the product was 24.1 mg, and its estimated purity by LCMS analysis was 92%. Analysis condition 1: Retention time=1.61 min; ESI-MS(+) m/z (M+3H)³⁺: 1092.6.

### Preparation of Example 5280

Example 5280 was prepared on a 50 μmol scale. The yield of the product was 5.1 mg, and its estimated purity by LCMS analysis was 90.4%. Analysis condition 1: Retention time=1.59 min; ESI-MS(+) m/z (M+3H)³⁺: 1130.8.

### Preparation of Example 5281

Example 5281 was prepared on a 50 μmol scale. The yield of the product was 7.6 mg, and its estimated purity by LCMS analysis was 91%. Analysis condition 1: Retention time=1.47 min; ESI-MS(+) m/z (M+3H)³⁺: 770.3.

### Preparation of Example 5282

Example 5282 was prepared on a 50 μmol scale. The yield of the product was 18 mg, and its estimated purity by LCMS analysis was 97.2%. Analysis condition 1: Retention time=1.62 min; ESI-MS(+) m/z (M+3H)³⁺: 1048.

### Preparation of Example 5283

Example 5283 was prepared on a 50 μmol scale. The yield of the product was 11.4 mg, and its estimated purity by LCMS analysis was 93.6%. Analysis condition 2: Retention time=1.7 min; ESI-MS(+) m/z (M+3H)³⁺: 1061.1.

### Preparation of Example 5284

Example 5284 was prepared on a 50 μmol scale. The yield of the product was 17 mg, and its estimated purity by LCMS analysis was 92.7%. Analysis condition 2: Retention time=1.6 min; ESI-MS(+) m/z (M+3H)³⁺: 1075.2.

### Preparation of Example 5285

Example 5285 was prepared on a 50 μmol scale. The yield of the product was 26.8 mg, and its estimated purity by LCMS analysis was 93.8%. Analysis condition 2: Retention time=1.64 min; ESI-MS(+) m/z (M+3H)³⁺: 1075.

### Preparation of Example 5286

Example 5286 was prepared on a 50 μmol scale. The yield of the product was 14.2 mg, and its estimated purity by LCMS analysis was 93.9%. Analysis condition 1: Retention time=1.73 min; ESI-MS(+) m/z (M+3H)³⁺: 1074.1.

### Preparation of Example 5287

Example 5287 was prepared on a 50 μmol scale. The yield of the product was 22.3 mg, and its estimated purity by LCMS analysis was 86.8%. Analysis condition 2: Retention time=1.63 min; ESI-MS(+) m/z (M+3H)³⁺: 1084.2.

### Preparation of Example 5288

Example 5288 was prepared on a 50 μmol scale. The yield of the product was 7.6 mg, and its estimated purity by LCMS analysis was 90.1%. Analysis condition 2: Retention time=1.66 min; ESI-MS(+) m/z (M+3H)³⁺: 1073.9.

### Preparation of Example 5289

Example 5289 was prepared on a 50 μmol scale. The yield of the product was 13.5 mg, and its estimated purity by LCMS analysis was 88.4%. Analysis condition 1: Retention time=1.73 min; ESI-MS(+) m/z (M+3H)³⁺: 1063.9.

### Preparation of Example 5290

Example 5290 was prepared on a 50 μmol scale. The yield of the product was 10 mg, and its estimated purity by LCMS analysis was 92.8%. Analysis condition 2: Retention time=1.61 min; ESI-MS(+) m/z (M+3H)³⁺: 1084.1.

### Preparation of Example 5291

Example 5291 was prepared on a 50 μmol scale. The yield of the product was 15.5 mg, and its estimated purity by LCMS analysis was 94.1%. Analysis condition 2: Retention time=1.62 min; ESI-MS(+) m/z (M+3H)³⁺: 1073.5.

### Preparation of Example 5292

Example 5292 was prepared on a 50 μmol scale. The yield of the product was 15 mg, and its estimated purity by LCMS analysis was 87.3%. Analysis condition 2: Retention time=1.64 min; ESI-MS(+) m/z (M+3H)³⁺: 1091.2.

### Preparation of Example 5293

Example 5293 was prepared on a 50 μmol scale. The yield of the product was 16.7 mg, and its estimated purity by LCMS analysis was 87%. Analysis condition 2: Retention time=1.56 min; ESI-MS(+) m/z (M+3H)³⁺: 1066.2.

### Preparation of Example 5294

Example 5294 was prepared on a 50 μmol scale. The yield of the product was 1.9 mg, and its estimated purity by LCMS analysis was 85.5%. Analysis condition 1: Retention time=1.62 min; ESI-MS(+) m/z (M+3H)³⁺: 1130.9.

### Preparation of Example 5295

Example 5295 was prepared on a 15 μmol scale. The yield of the product was 6.4 mg, and its estimated purity by LCMS analysis was 94.1%. Analysis condition 1: Retention time=1.76 min; ESI-MS(+) m/z (M+3H)³⁺: 1155.

### Preparation of Example 5296

Example 5296 was prepared on a 80 μmol scale. The yield of the product was 12.9 mg, and its estimated purity by LCMS analysis was 98.2%. Analysis condition 1: Retention time=1.8 min: ESI-MS(+) m/z (M+3H)³⁺: 1202.6.

### Preparation of Example 5297

Example 5297 was prepared on a 50 μmol scale. The yield of the product was 7.2 mg, and its estimated purity by LCMS analysis was 83.8%. Analysis condition 2: Retention time=1.51 min; ESI-MS(+) m/z (M+3H)³⁺: 1033.

### Preparation of Example 5298

Example 5298 was prepared on a 50 μmol scale. The yield of the product was 6.2 mg, and its estimated purity by LCMS analysis was 84.8%. Analysis condition 1: Retention time=1.55 min; ESI-MS(+) m/z (M+3H)³⁺: 1048.3.

### Biological Activity

The ability of the compounds of formula (I) to bind to PD-1 was investigated using a Jurkat-PD-1 Cell Binding High-Content Screening Assay or HTRF Assay.

### Jurkat-PD-1 Cell Binding High-Content Screening Assay (CBA): Method 1

Phycoerythrin (PE) was covalently linked to the Ig epitope tag of human PD-L1-Ig and fluorescently-labeled PD-L1-Ig was used for binding studies with a Jurkat cell line over-expressing human PD-1 (Jurkat-PD-1). Briefly, 8×10³ Jurkat-hPD-1 cells were seeded into 384 well plates in 20 μl of DMEM supplemented with 10% fetal calf serum. 100 nl of compound was added to cells followed by incubation at 37° C. for 2 hours. Then, 5 μl of PE-labeled PD-L1-Ig (20 nM final), diluted in DMEM supplemented with 10% fetal calf serum. After 1 hour incubation, cells were fixed with 4% paraformaldehyde in dPBS containing 10 μg/ml Hoechst 33342 and then washed 3× in 100 μl dPBS. Data was collected and processed using a Cell Insight NXT High Content Imager and associated software.

### Protein Sequence Information

hPDL1(18-239)-TVMV-mIgG1(221-447)-C225S
(SEQ ID NO: 1)

| | |
|---|---|
| 1 | AFTVTVPKDL YVVEYGSNMT IECKFPVEKQ |
| | LDLAALIVYW EMEDKNIIQF |
| 51 | VHGEEDLKVQ HSSYRQRARL LKDQLSLGNA |
| | ALQITDVKLQ DAGVYRCMIS |
| 101 | YGGADYKRIT VKVNAPYNKI NQRILVVDPV |
| | TSEHELTCQA EGYPKAEVIW |
| 151 | TSSDHQVLSG KTTTTNSKRE EKLFNVTSTL |
| | RINTTTNEIF YCTFRRLDPE |
| 201 | ENHTAELVIP ELPLAHPPNE RTGSPGGGGG |
| | RETVRFQGGT GDAVPRDSGC |
| 251 | KPCICTVPEV SSVFIFPPKP KDVLTITLTP |
| | KVTCVVVDIS KDDPEVQFSW |
| 301 | FVDDVEVHTA QTQPREEQFN STFRSVSELP |
| | IMHQDWLNGK EFKCRVNSAA |
| 351 | FPAPIEKTIS KTKGRPKAPQ VYTIPPPKEQ |
| | MAKDKVSLTC MITDFFPEDI |
| 401 | TVEWQWNGQP AENYKNTQPI MDTDGSYFVY |
| | SKLNVQKSNW EAGNTFTCSV |
| 451 | LHEGLHNHHT EKSLSHSPGK |

Jurkat HPDL1 PD1 IC₅₀ (μM) is presented in Table 3.

TABLE 3

| Compound | Jurkat HPDL1 PD1 IC₅₀ |
|---|---|
| Number | (uM) (Method 1) |
| 1000 | 0.86 |
| 1001 | 0.33 |
| 1002 | 0.35 |
| 1003 | 0.14 |
| 1004 | 0.25 |
| 1005 | 0.01 |
| 1006 | 0.60 |
| 1007 | 0.26 |
| 1008 | 0.70 |
| 1009 | 0.09 |
| 1010 | 0.16 |
| 1011 | 0.10 |
| 1012 | 0.13 |
| 1013 | 0.16 |
| 1014 | 0.20 |
| 1015 | 0.16 |
| 1016 | 0.13 |
| 1017 | 0.04 |
| 1018 | 0.12 |
| 1019 | 0.36 |
| 1020 | 0.07 |
| 1021 | 0.14 |
| 1022 | 0.64 |
| 1023 | 0.14 |
| 1024 | 0.15 |
| 1025 | 0.12 |
| 1026 | 0.13 |
| 1027 | 0.21 |
| 1028 | 0.11 |
| 1029 | 0.04 |
| 1030 | 0.03 |
| 1031 | 0.09 |
| 1032 | 0.51 |
| 1033 | 0.03 |
| 1034 | 0.05 |
| 1035 | 0.08 |
| 1036 | 0.05 |
| 1037 | 0.14 |
| 1038 | 0.13 |
| 1039 | 0.08 |
| 1040 | 0.17 |
| 1041 | 0.31 |
| 1042 | 0.04 |
| 1043 | 0.17 |
| 1044 | 0.04 |
| 1045 | 0.20 |
| 1046 | 0.10 |
| 1047 | 0.07 |
| 1048 | 0.06 |
| 1049 | 0.01 |
| 1050 | 0.11 |
| 1051 | 0.15 |
| 1052 | 0.04 |
| 1053 | 0.12 |
| 1054 | 0.09 |
| 1055 | 0.11 |
| 1056 | 0.06 |
| 1057 | 0.18 |
| 1058 | 0.91 |
| 1059 | 0.21 |
| 1060 | 0.11 |
| 1061 | 0.10 |
| 1062 | 0.15 |
| 1063 | 0.06 |
| 1064 | 0.10 |
| 1065 | 0.11 |
| 1066 | 0.79 |
| 1067 | 0.12 |
| 1068 | 0.17 |
| 1069 | 0.22 |
| 1070 | 0.63 |
| 1071 | 0.13 |
| 1072 | 0.18 |
| 1073 | 0.13 |
| 1074 | 0.11 |
| 1075 | 0.55 |
| 1076 | 0.05 |
| 1077 | 0.05 |
| 1078 | 0.48 |
| 1079 | 0.25 |
| 1080 | 0.14 |
| 1081 | 0.06 |
| 1082 | 0.04 |
| 1083 | 0.25 |
| 1084 | 0.09 |
| 1085 | 0.07 |
| 1086 | 0.69 |
| 1087 | 0.33 |
| 1088 | 0.41 |
| 1089 | 0.94 |
| 1090 | 1.00 |
| 1091 | 0.09 |
| 1092 | 0.42 |
| 1093 | 0.05 |
| 1094 | 0.77 |
| 1095 | 0.15 |
| 1096 | 0.29 |
| 1097 | 0.21 |
| 1098 | 0.13 |
| 1099 | 0.03 |
| 1100 | 0.53 |
| 1101 | 0.29 |
| 1102 | 0.30 |
| 1103 | 0.69 |
| 1104 | 0.13 |
| 1105 | 0.87 |
| 1106 | 0.07 |
| 1107 | 0.07 |
| 1108 | 0.53 |
| 1109 | 0.12 |
| 1110 | 0.04 |
| 1111 | 0.07 |
| 1112 | 0.10 |
| 1113 | 0.14 |
| 1114 | 0.93 |
| 1115 | 0.05 |
| 1116 | 0.06 |
| 1117 | 0.36 |
| 1118 | 0.16 |
| 1119 | 0.03 |
| 1120 | 0.27 |
| 1121 | 0.06 |
| 1122 | 0.24 |
| 1123 | 0.06 |
| 1124 | 0.69 |
| 1125 | 0.05 |
| 1126 | 0.23 |
| 1127 | 0.09 |
| 1128 | 0.08 |
| 1129 | 0.09 |
| 1130 | 0.18 |
| 1131 | 0.28 |
| 1132 | 0.16 |
| 1133 | 0.35 |
| 1134 | 0.11 |
| 1135 | 0.14 |
| 1136 | 0.21 |
| 1137 | 0.27 |
| 1138 | 0.07 |
| 1139 | 0.06 |
| 1140 | 0.03 |
| 1141 | 0.04 |
| 1142 | 0.24 |
| 1143 | 0.24 |
| 1144 | 0.05 |
| 1145 | 0.02 |
| 1146 | 0.06 |
| 1147 | 0.02 |
| 1148 | 0.05 |
| 1149 | 0.07 |
| 1150 | 0.05 |
| 1151 | 0.04 |
| 1152 | 0.08 |
| 1153 | 0.19 |
| 1154 | 0.03 |
| 1155 | 0.02 |
| 1156 | 0.03 |
| 1157 | 0.11 |
| 1158 | 0.03 |
| 1159 | 0.07 |
| 1160 | 0.03 |
| 1161 | 0.11 |
| 1162 | 0.22 |
| 1163 | 0.04 |
| 1164 | 0.07 |
| 1165 | 0.15 |
| 1166 | 0.06 |
| 1167 | 0.10 |
| 1168 | 0.12 |
| 1169 | 0.06 |
| 1170 | 0.05 |
| 1171 | 0.15 |
| 1172 | 0.29 |
| 1173 | 0.22 |
| 1174 | 0.06 |
| 1175 | 0.12 |
| 1176 | 0.07 |
| 1177 | 0.12 |
| 1178 | 0.05 |
| 1179 | 0.07 |
| 1180 | 0.51 |
| 1181 | 0.34 |
| 1182 | 0.30 |
| 1183 | 0.15 |
| 1184 | 0.13 |
| 1185 | 0.35 |
| 1186 | 0.93 |
| 1187 | 0.05 |
| 1188 | 0.09 |
| 1189 | 0.03 |
| 1190 | 0.02 |
| 1191 | 0.07 |
| 1192 | 0.03 |
| 1193 | 0.35 |
| 1194 | 0.69 |
| 1195 | 0.32 |
| 1196 | 0.24 |
| 1197 | 0.36 |
| 1198 | 0.06 |
| 1199 | 0.45 |
| 1200 | 0.49 |
| 1201 | 0.56 |
| 1202 | 0.73 |
| 1203 | 0.10 |
| 1204 | 0.33 |
| 1205 | 0.81 |
| 1206 | 0.59 |
| 1207 | 0.07 |
| 1208 | 0.14 |
| 1209 | 0.14 |
| 1210 | 0.88 |
| 1211 | 0.37 |
| 1212 | 0.69 |
| 1213 | 0.25 |
| 1214 | 0.05 |
| 1215 | 0.08 |
| 1216 | 0.03 |
| 1217 | 0.06 |
| 1218 | 0.20 |
| 1219 | 0.26 |
| 1220 | 0.26 |
| 1221 | 0.22 |
| 1222 | 0.12 |
| 1223 | 0.32 |
| 1224 | 0.04 |
| 1225 | 0.29 |
| 1226 | 0.03 |
| 1227 | 0.04 |
| 1228 | 0.90 |
| 1229 | 0.13 |
| 1230 | 0.01 |
| 1231 | 0.04 |
| 1232 | 0.12 |
| 1233 | 0.05 |
| 1234 | 0.03 |
| 1235 | 0.06 |
| 1236 | 0.11 |
| 1237 | 0.03 |
| 1238 | 0.03 |
| 1239 | 0.08 |
| 1240 | 0.04 |
| 1241 | 0.02 |
| 1242 | 0.09 |
| 1243 | 0.01 |
| 1244 | 0.04 |
| 1245 | 0.10 |
| 1246 | 0.05 |
| 1247 | 0.06 |
| 1248 | 0.07 |
| 1249 | 0.06 |
| 1250 | 0.07 |
| 1251 | 0.04 |
| 1252 | 0.09 |
| 1253 | 0.03 |
| 1254 | 0.03 |
| 1255 | 0.03 |
| 1256 | 0.06 |
| 1257 | 0.13 |
| 1258 | 0.04 |
| 1259 | 0.11 |
| 1260 | 0.17 |
| 1261 | 0.08 |
| 1262 | 4.1 nM |
| 1263 | 0.89 |
| 1264 | 4.9 nM |
| 1265 | 0.01 |
| 1266 | 0.03 |
| 1267 | 0.26 |
| 1268 | 0.11 |
| 1269 | 0.03 |
| 1270 | 0.11 |
| 1271 | 0.03 |
| 1272 | 0.16 |
| 1273 | 0.01 |
| 1274 | 0.03 |
| 1275 | 0.03 |
| 1276 | 0.03 |
| 1277 | 0.02 |
| 1278 | 0.01 |
| 1279 | 0.02 |
| 1280 | 0.08 |
| 1281 | 0.33 |
| 1282 | 0.13 |
| 1283 | 0.04 |
| 1284 | 0.01 |
| 1285 | 0.05 |
| 1286 | 0.01 |
| 1287 | 0.01 |
| 1288 | 0.08 |
| 1289 | 0.09 |
| 1290 | 0.01 |
| 1291 | 0.02 |
| 1292 | 0.03 |
| 1293 | 0.07 |
| 1294 | 0.30 |
| 1295 | 0.02 |
| 1296 | 0.01 |
| 1297 | 0.32 |
| 1298 | 0.03 |
| 1299 | 0.02 |
| 1300 | 0.02 |
| 1301 | 0.06 |
| 1302 | 0.02 |
| 1303 | 0.07 |
| 1304 | 0.02 |
| 1305 | 0.20 |
| 1306 | 0.05 |
| 1307 | 0.10 |
| 1308 | 0.11 |
| 1309 | 0.09 |
| 1310 | 0.11 |
| 1311 | 0.05 |
| 1312 | 0.05 |
| 1313 | 0.04 |
| 1314 | 0.04 |
| 1315 | 0.06 |
| 1316 | 0.05 |
| 1317 | 0.04 |
| 1318 | 0.02 |
| 1319 | 0.04 |
| 1320 | 0.02 |
| 1321 | 0.08 |
| 1322 | 0.02 |
| 1323 | 3.0 nM |
| 1324 | 0.03 |
| 1325 | 0.01 |
| 1326 | 0.02 |
| 1327 | 0.08 |
| 1328 | 0.01 |
| 1329 | 0.19 |
| 1330 | 0.04 |
| 1331 | 0.01 |
| 1332 | 0.03 |
| 1333 | 0.02 |
| 1334 | 0.63 |
| 1335 | 5.7 nM |
| 1336 | 0.01 |
| 1337 | 0.28 |
| 1338 | 0.01 |
| 1339 | 0.05 |
| 1340 | 0.02 |
| 1341 | 0.05 |
| 1342 | 0.06 |
| 1343 | 0.04 |
| 1344 | 0.07 |
| 1345 | 0.01 |
| 1346 | 0.07 |
| 1347 | 0.03 |
| 1348 | 0.05 |
| 1349 | 0.06 |
| 1350 | 0.03 |
| 1351 | 0.05 |
| 1352 | 0.01 |
| 1353 | 0.03 |
| 1354 | 0.02 |
| 1355 | 0.13 |
| 1356 | 0.02 |
| 1357 | 0.06 |
| 1358 | 0.01 |
| 1359 | 0.05 |
| 1360 | 0.02 |
| 1361 | 0.01 |
| 1362 | 0.03 |
| 1363 | 0.11 |
| 1364 | 0.01 |
| 1365 | 0.01 |
| 1366 | 0.03 |
| 1367 | 0.39 |
| 1368 | 0.13 |
| 1369 | 0.03 |
| 1370 | 0.01 |
| 1371 | 0.03 |
| 1372 | 0.14 |
| 1373 | 0.03 |
| 1374 | 0.04 |
| 1375 | 0.05 |
| 1376 | 0.02 |
| 1377 | 0.02 |
| 1378 | 0.01 |
| 1379 | 0.11 |
| 1380 | 0.02 |
| 1381 | 0.03 |
| 1382 | 0.08 |
| 1383 | 0.06 |
| 1384 | 0.02 |
| 1385 | 4.7 nM |
| 1386 | 0.05 |
| 1387 | 0.03 |
| 1388 | 0.06 |
| 1389 | 0.03 |
| 1390 | 0.11 |
| 1391 | 0.03 |
| 1392 | 0.04 |
| 1393 | 0.03 |
| 1394 | 0.01 |
| 1395 | 0.02 |
| 1396 | 0.01 |
| 1397 | 0.02 |
| 1398 | 3.7 nM |
| 1399 | 0.14 |
| 1400 | 0.02 |
| 1401 | 0.01 |
| 1402 | 0.02 |
| 1403 | 0.13 |
| 1404 | 0.12 |
| 1405 | 0.03 |
| 1406 | 0.02 |
| 1407 | 0.04 |
| 1408 | 0.10 |
| 1409 | 0.04 |
| 1410 | 0.02 |
| 1411 | 0.02 |
| 1412 | 0.05 |
| 1413 | 0.03 |
| 1414 | 0.10 |
| 1415 | 0.04 |
| 1416 | 4.7 nM |
| 1417 | 4.9 nM |
| 1418 | 0.01 |
| 1419 | 0.01 |
| 1420 | 0.01 |
| 1421 | 0.01 |
| 1422 | 0.05 |
| 1423 | 0.07 |
| 1424 | 0.05 |
| 1425 | 0.14 |
| 1426 | 0.01 |
| 1427 | 0.11 |
| 1428 | 0.04 |
| 1429 | 0.01 |
| 1430 | 0.01 |
| 1431 | 0.01 |
| 1432 | 0.05 |
| 1433 | 0.16 |
| 1434 | 0.05 |
| 1435 | 0.05 |
| 1436 | 0.06 |
| 1437 | 0.02 |
| 1438 | 0.07 |
| 1439 | 0.06 |
| 1440 | 0.19 |
| 1441 | 0.09 |
| 1442 | 0.03 |
| 1443 | 0.01 |
| 1444 | 0.08 |
| 1445 | 0.11 |
| 1446 | 0.08 |
| 1447 | 0.03 |
| 1448 | 0.06 |
| 1449 | 0.17 |
| 1450 | 0.06 |
| 1451 | 0.08 |
| 1452 | 0.04 |
| 1453 | 0.08 |
| 1454 | 0.02 |
| 1455 | 0.08 |
| 1456 | 0.04 |
| 1457 | 0.01 |
| 1458 | 0.04 |
| 1459 | 0.04 |
| 1460 | 0.02 |
| 1461 | 0.23 |
| 1462 | 0.01 |
| 1463 | 5.9 nM |
| 1464 | 0.01 |
| 1465 | 5.8 nM |
| 1466 | 0.02 |
| 1467 | 0.02 |
| 1468 | 5.0 nM |
| 1469 | 0.01 |
| 1470 | 5.7 nM |
| 1471 | 0.01 |
| 1472 | 0.01 |
| 1473 | 0.04 |
| 1474 | 0.08 |
| 1475 | 0.05 |
| 1476 | 0.06 |
| 1477 | 0.02 |
| 1478 | 0.02 |
| 1479 | 0.02 |
| 1480 | 0.04 |
| 1481 | 0.12 |
| 1482 | 0.11 |
| 1483 | 0.06 |
| 1484 | 0.07 |
| 1485 | 0.39 |
| 1486 | 0.62 |
| 1487 | 0.32 |
| 1488 | 0.04 |
| 1489 | 0.04 |
| 1490 | 0.07 |
| 1491 | 0.09 |
| 1492 | 0.04 |
| 1493 | 0.03 |
| 1494 | 0.05 |
| 1495 | 0.04 |
| 1496 | 0.18 |
| 1497 | 0.20 |
| 1498 | 0.12 |
| 1499 | 0.14 |
| 1500 | 0.08 |
| 1501 | 0.08 |
| 1502 | 0.10 |
| 1503 | 0.01 |
| 1504 | 0.02 |
| 1505 | 0.02 |
| 1506 | 0.02 |
| 1507 | 0.01 |
| 1508 | 0.02 |
| 1509 | 0.02 |
| 1510 | 0.06 |
| 1511 | 0.01 |
| 1512 | 0.02 |
| 1513 | 0.02 |
| 1514 | 0.01 |
| 1515 | 0.02 |
| 1516 | 0.02 |
| 1517 | 0.02 |
| 1518 | 0.05 |
| 1519 | 0.02 |
| 1520 | 0.01 |
| 1521 | 0.07 |
| 1522 | 0.05 |
| 1523 | 5.6 nM |
| 1524 | 0.01 |
| 1525 | 0.03 |
| 1526 | 0.01 |
| 1527 | 0.01 |
| 1528 | 0.01 |
| 1529 | 0.05 |
| 1530 | 0.03 |
| 1531 | 0.11 |
| 1532 | 0.17 |
| 1533 | 0.15 |
| 1534 | 0.49 |
| 1535 | 0.36 |
| 1536 | 0.01 |
| 1537 | 0.02 |
| 1538 | 0.10 |
| 1539 | 0.10 |
| 1540 | 0.03 |
| 1541 | 0.02 |
| 1542 | 0.04 |
| 1543 | 0.01 |
| 1544 | 0.01 |
| 1545 | 0.04 |
| 1546 | 0.02 |
| 1547 | 0.01 |
| 1548 | 0.01 |
| 1549 | 0.04 |
| 1550 | 0.01 |
| 1551 | 0.01 |
| 1552 | 0.01 |
| 1553 | 0.01 |
| 1554 | 2.8 nM |
| 1555 | 4.6 nM |
| 1556 | 5.1 nM |
| 1557 | 0.01 |
| 1558 | 3.7 nM |
| 1559 | 2.3 nM |
| 1560 | 0.02 |
| 1561 | 0.03 |
| 1562 | 0.03 |
| 1563 | 0.02 |
| 1564 | 0.01 |
| 1565 | 0.01 |
| 1566 | 0.04 |
| 1567 | 0.02 |
| 1568 | 0.01 |
| 1569 | 0.04 |
| 1570 | 0.01 |
| 1571 | 0.01 |
| 1572 | 0.01 |
| 1573 | 0.05 |
| 1574 | 0.02 |
| 1575 | 0.05 |
| 1576 | 0.11 |
| 1577 | 0.03 |
| 1578 | 0.01 |
| 1579 | 0.02 |
| 1580 | 0.01 |
| 1581 | 3.9 nM |
| 1582 | 0.02 |
| 1583 | 0.01 |
| 1584 | 0.02 |
| 1585 | 0.73 |
| 1586 | 0.54 |
| 1587 | 0.70 |
| 1588 | 0.32 |
| 1589 | 0.03 |
| 1590 | 4.7 nM |
| 1591 | 0.03 |
| 1592 | 0.01 |
| 1593 | 2.4 nM |
| 1594 | 0.01 |
| 1595 | 0.01 |
| 1596 | 0.02 |
| 1597 | 0.05 |
| 1598 | 0.01 |
| 1599 | 0.01 |
| 1600 | 0.01 |
| 1601 | 0.02 |
| 1602 | 0.01 |
| 1603 | 0.01 |
| 1604 | 0.01 |
| 1605 | 0.01 |
| 1606 | 0.02 |
| 1607 | 0.02 |
| 1608 | 0.02 |
| 1609 | 0.11 |
| 1610 | 0.02 |
| 1611 | 0.04 |
| 1612 | 0.02 |
| 1613 | 0.05 |
| 1614 | 0.01 |
| 1615 | 0.01 |
| 1616 | 0.01 |
| 1617 | 0.01 |
| 1618 | 0.04 |
| 1619 | 0.02 |
| 1620 | 0.01 |
| 1621 | 0.02 |
| 1622 | 0.02 |
| 1623 | 0.19 |
| 1624 | 3.9 nM |
| 1625 | 0.01 |
| 1626 | 4.2 nM |
| 1627 | 0.06 |
| 1628 | 0.09 |
| 1629 | 0.05 |
| 1630 | 0.03 |
| 1631 | 0.11 |
| 1632 | 0.13 |
| 1633 | 0.05 |
| 1634 | 0.01 |
| 1635 | 0.01 |
| 1636 | 0.01 |
| 1637 | 0.15 |
| 1638 | 0.07 |
| 1639 | 0.06 |
| 1640 | 0.10 |
| 1641 | 0.14 |
| 1642 | 0.18 |
| 1643 | 0.08 |
| 1644 | 0.06 |
| 1645 | 0.01 |
| 1646 | 0.03 |
| 1647 | 0.04 |
| 1648 | 0.03 |
| 1649 | 0.10 |
| 1650 | 0.08 |
| 1651 | 0.10 |
| 1652 | 0.03 |
| 1653 | 0.03 |
| 1654 | 0.08 |
| 1655 | 0.03 |
| 1656 | 0.03 |
| 1657 | 0.05 |
| 1658 | 0.03 |
| 1659 | 0.03 |
| 1660 | 0.02 |
| 1661 | 0.05 |
| 1662 | 0.01 |
| 1663 | 0.01 |
| 1664 | 0.05 |
| 1665 | 0.09 |
| 1666 | 0.09 |
| 1667 | 0.11 |
| 1668 | 0.05 |
| 1669 | 0.06 |
| 1670 | 0.08 |
| 1671 | 0.09 |
| 1672 | 0.09 |
| 1673 | 0.09 |
| 1674 | 0.07 |
| 1675 | 0.12 |
| 1676 | 0.24 |
| 1677 | 0.16 |
| 1678 | 0.15 |
| 1679 | 0.23 |
| 1680 | 0.17 |
| 1681 | 0.03 |
| 1682 | 0.09 |
| 1683 | 0.03 |
| 1684 | 0.01 |
| 1685 | 0.10 |
| 1686 | 0.16 |
| 1687 | 0.11 |
| 1688 | 1.0 |
| 1689 | 0.02 |
| 1690 | 0.01 |
| 1691 | 0.02 |
| 1692 | 0.08 |
| 1693 | 0.02 |
| 1694 | 0.10 |
| 1695 | 0.01 |
| 1696 | 0.01 |
| 1697 | 0.02 |
| 1698 | 0.10 |
| 1699 | 0.04 |
| 1700 | 0.18 |
| 1701 | 0.01 |
| 1702 | 0.02 |
| 1703 | 0.02 |
| 1704 | 0.15 |
| 1705 | 0.03 |
| 1706 | 0.12 |
| 1707 | 4.2 nM |
| 1708 | 5.8 nM |
| 1709 | 0.03 |
| 1710 | 0.11 |
| 1711 | 0.06 |
| 1712 | 0.24 |
| 1713 | 0.13 |
| 1714 | 0.02 |
| 1715 | 0.06 |
| 1716 | 0.01 |
| 1717 | 0.02 |
| 1718 | 0.03 |
| 1719 | 0.03 |
| 1720 | 0.04 |
| 1721 | 0.24 |
| 1722 | 0.01 |
| 1723 | 0.01 |
| 1724 | 0.01 |
| 1725 | 0.01 |
| 1726 | 0.01 |
| 1727 | 0.01 |
| 1728 | 0.01 |
| 1729 | 0.01 |
| 1730 | 0.01 |
| 1731 | 0.01 |
| 1732 | 0.01 |
| 1733 | 0.01 |
| 1734 | 0.01 |
| 1735 | 0.02 |
| 1736 | 0.02 |
| 1737 | 0.02 |
| 1738 | 0.02 |
| 1739 | 0.02 |
| 1740 | 0.02 |
| 1741 | 0.02 |
| 1742 | 0.02 |
| 1743 | 0.02 |
| 1744 | 0.02 |
| 1745 | 0.02 |
| 1746 | 0.02 |
| 1747 | 0.02 |
| 1748 | 0.02 |
| 1749 | 0.02 |
| 1750 | 0.02 |
| 1751 | 0.03 |
| 1752 | 0.03 |
| 1753 | 0.03 |
| 1754 | 0.03 |
| 1755 | 0.03 |
| 1756 | 0.03 |
| 1757 | 0.03 |
| 1758 | 0.03 |
| 1759 | 0.03 |
| 1760 | 0.03 |
| 1761 | 0.03 |
| 1762 | 0.04 |
| 1763 | 0.04 |
| 1764 | 0.04 |
| 1765 | 0.04 |
| 1766 | 0.04 |
| 1767 | 0.04 |
| 1768 | 0.04 |
| 1769 | 0.04 |
| 1770 | 0.04 |
| 1771 | 0.04 |
| 1772 | 0.04 |
| 1773 | 0.05 |
| 1774 | 0.05 |
| 1775 | 0.05 |
| 1776 | 0.06 |
| 1777 | 0.06 |
| 1778 | 0.07 |
| 1779 | 0.07 |
| 1780 | 0.08 |
| 1781 | 0.08 |
| 1782 | 0.08 |
| 1783 | 0.09 |
| 1784 | 0.09 |
| 1785 | 0.09 |
| 1786 | 0.10 |
| 1787 | 0.10 |
| 1788 | 0.11 |
| 1789 | 0.12 |
| 1790 | 0.13 |
| 1791 | 0.13 |
| 1792 | 0.13 |
| 1793 | 0.15 |
| 1794 | 0.15 |
| 1795 | 0.16 |
| 1796 | 0.16 |
| 1797 | 0.16 |
| 1798 | 0.17 |
| 1799 | 0.18 |
| 1800 | 0.18 |
| 1801 | 0.19 |
| 1802 | 0.19 |
| 1803 | 0.20 |
| 1804 | 0.21 |
| 1805 | 0.23 |
| 1806 | 0.23 |
| 1807 | 0.24 |
| 1808 | 0.86 |
| 1809 | 0.27 |
| 1810 | 0.28 |
| 1811 | 0.28 |
| 1812 | 0.28 |
| 1813 | 0.30 |
| 1814 | 0.30 |
| 1815 | 0.31 |
| 1816 | 0.32 |
| 1817 | 0.41 |
| 1818 | 0.42 |
| 1819 | 0.43 |
| 1820 | 0.48 |
| 1821 | 0.49 |
| 1822 | 0.58 |
| 1823 | 0.60 |
| 1824 | 0.63 |
| 1825 | 0.66 |
| 1826 | 0.76 |
| 1827 | 0.79 |
| 1828 | 0.82 |
| 1829 | 0.85 |
| 1830 | 0.90 |
| 1831 | 0.98 |
| 1832 | 0.24 |
| 1833 | 0.43 |
| 1834 | 0.01 |
| 1835 | 0.11 |
| 1836 | 0.67 |
| 1837 | 0.47 |
| 1838 | 0.01 |
| 1839 | 5.1 nM |
| 1840 | 0.05 |
| 1841 | 0.02 |
| 1842 | 0.02 |
| 1843 | 0.01 |
| 1844 | 0.02 |
| 1845 | 0.01 |
| 1846 | 0.02 |
| 1847 | 0.70 |
| 1848 | 0.06 |
| 1849 | 0.01 |
| 1850 | 0.01 |
| 1851 | 0.60 |
| 1852 | 0.02 |
| 1853 | 0.03 |
| 1854 | 0.02 |
| 1855 | 0.01 |
| 1856 | 0.02 |
| 1857 | 0.01 |
| 1858 | 0.01 |
| 1859 | 0.05 |
| 1860 | 0.01 |
| 1861 | 0.10 |
| 1862 | 0.04 |
| 1863 | 0.01 |
| 1864 | 0.04 |
| 1865 | 0.02 |
| 1866 | 0.05 |
| 1867 | 0.03 |
| 1868 | 0.17 |
| 1869 | 0.05 |
| 1870 | 0.01 |
| 1871 | 5.3 nM |
| 1872 | 0.02 |
| 1873 | 0.03 |
| 1874 | 0.28 |
| 1875 | 0.05 |
| 1876 | 0.34 |
| 1877 | 0.01 |
| 1878 | 0.01 |
| 1879 | 0.01 |
| 1880 | 0.05 |
| 1881 | 0.10 |
| 1882 | 0.03 |
| 1883 | 0.06 |
| 1884 | 0.03 |
| 1885 | 0.11 |
| 1886 | 0.14 |
| 1887 | 0.25 |
| 1888 | 0.20 |
| 1889 | 0.03 |
| 1890 | 0.15 |
| 1891 | 0.15 |
| 1892 | 0.06 |
| 1893 | 0.26 |
| 1894 | 0.30 |
| 1895 | 0.11 |
| 1896 | 0.19 |
| 1897 | 0.01 |
| 1898 | 0.07 |
| 1899 | 0.01 |
| 1900 | 0.05 |
| 1901 | 0.01 |
| 1902 | 0.01 |
| 1903 | 0.04 |
| 1904 | 0.01 |
| 1905 | 0.01 |
| 1906 | 0.05 |
| 1907 | 0.12 |
| 1908 | 0.04 |
| 1909 | 0.01 |
| 1910 | 0.02 |
| 1911 | 0.01 |
| 1912 | 0.02 |
| 1913 | 0.01 |
| 1914 | 0.01 |
| 1915 | 0.01 |
| 1916 | 0.02 |
| 1917 | 0.04 |
| 1918 | 0.27 |
| 1919 | 0.03 |
| 1920 | 0.01 |
| 1921 | 0.01 |
| 1922 | 0.04 |
| 1923 | 0.02 |
| 1924 | 0.04 |
| 1925 | 0.03 |
| 1926 | 0.01 |
| 1927 | 0.01 |
| 1928 | 0.02 |
| 1929 | 0.02 |
| 1930 | 0.02 |
| 1931 | 0.02 |
| 1932 | 0.02 |
| 1933 | 0.01 |
| 1934 | 0.01 |
| 1935 | 0.04 |
| 1936 | 0.34 |
| 1937 | 0.07 |
| 1938 | 0.34 |
| 1939 | 0.11 |
| 1940 | 0.02 |
| 1941 | 0.01 |
| 1942 | 0.01 |
| 1943 | 0.01 |
| 1944 | 0.90 |
| 1945 | 0.93 |
| 1946 | 0.39 |
| 1947 | 0.43 |
| 1948 | 0.02 |
| 2000 | 0.02 |
| 2001 | 0.11 |
| 2002 | 0.14 |
| 2003 | 0.02 |
| 2004 | 0.02 |
| 2005 | 0.02 |
| 2006 | 0.04 |
| 2007 | 0.01 |
| 2008 | 0.04 |
| 2009 | 0.02 |
| 2010 | 0.03 |
| 2011 | 0.03 |
| 2012 | 0.01 |
| 2013 | 0.01 |
| 2014 | 0.02 |
| 2015 | 0.04 |
| 2016 | 0.10 |
| 2017 | 0.02 |
| 2018 | 0.12 |
| 2019 | 0.28 |
| 2020 | 0.14 |
| 2021 | 0.01 |
| 2022 | 0.01 |
| 2023 | 0.17 |
| 2024 | 0.01 |
| 2025 | 0.26 |
| 2026 | 0.12 |
| 2027 | 0.02 |
| 2028 | 0.48 |
| 2029 | 0.04 |
| 2030 | 0.03 |
| 2031 | 0.07 |
| 2032 | 0.05 |
| 2033 | 0.03 |
| 2034 | 0.06 |
| 2035 | 0.04 |
| 2036 | 0.04 |
| 2037 | 0.02 |
| 2038 | 0.07 |
| 2039 | 0.08 |
| 2040 | 0.05 |
| 2041 | 0.02 |
| 2042 | 4.5 nM |
| 2043 | 0.01 |
| 2044 | 0.02 |
| 2045 | 0.01 |
| 2046 | 0.02 |
| 2047 | 0.06 |
| 2048 | 0.03 |
| 2049 | 0.09 |
| 2050 | 0.01 |
| 2051 | 0.01 |
| 2052 | 0.02 |
| 2053 | 0.03 |
| 2054 | 0.02 |
| 2055 | 0.95 |
| 2056 | 0.04 |
| 2057 | 0.04 |
| 2058 | 0.04 |
| 2059 | 0.74 |
| 2060 | 0.28 |
| 2061 | 0.01 |
| 2062 | 0.02 |
| 2063 | 0.05 |
| 2064 | 0.04 |
| 2065 | 0.01 |
| 2066 | 0.35 |
| 2067 | 3.8 nM |
| 2068 | 0.03 |
| 2069 | 0.10 |
| 2070 | 0.05 |
| 2071 | 0.09 |
| 2072 | 0.02 |
| 2073 | 0.01 |
| 2074 | 0.03 |
| 2075 | 0.09 |
| 2076 | 0.16 |
| 2077 | 0.03 |
| 2078 | 0.02 |
| 2079 | 0.02 |
| 2080 | 0.15 |
| 2081 | 0.03 |
| 2082 | 0.07 |
| 2083 | 0.06 |
| 2084 | 0.07 |
| 2085 | 0.01 |
| 2086 | 0.20 |
| 2087 | 0.05 |
| 2088 | 0.17 |
| 2089 | 0.05 |
| 2090 | 0.14 |
| 2091 | 0.03 |
| 2092 | 0.35 |
| 2093 | 0.11 |
| 2094 | 0.02 |
| 2095 | 0.04 |
| 2096 | 0.04 |
| 2097 | 0.03 |
| 2098 | 0.01 |
| 2099 | 0.02 |
| 2100 | 0.03 |
| 2101 | 0.11 |
| 2102 | 0.19 |
| 2103 | 0.04 |
| 2104 | 0.05 |
| 2105 | 0.10 |
| 2106 | 0.06 |
| 2107 | 0.24 |
| 2108 | 0.11 |
| 2109 | 0.20 |
| 2110 | 0.01 |
| 2111 | 0.01 |
| 2112 | 0.02 |
| 2113 | 0.02 |
| 2114 | 0.02 |
| 2115 | 0.01 |
| 2116 | 0.01 |
| 2117 | 0.02 |
| 2118 | 0.05 |
| 2119 | 0.02 |
| 2120 | 0.08 |
| 2121 | 0.04 |
| 2122 | 0.05 |
| 2123 | 0.02 |
| 2124 | 0.04 |
| 2125 | 0.04 |
| 2126 | 0.66 |
| 2127 | 0.01 |
| 2128 | 0.01 |
| 2129 | 0.01 |
| 2130 | 0.03 |
| 2131 | 0.07 |
| 2132 | 0.07 |
| 2133 | 0.01 |
| 2134 | 5.9 nM |
| 2135 | 0.01 |
| 2136 | 0.01 |
| 2137 | 0.03 |
| 2138 | 0.03 |
| 2139 | 0.02 |
| 2140 | 0.10 |
| 2141 | 0.02 |
| 2142 | 0.18 |
| 2143 | 0.02 |
| 2144 | 0.01 |
| 2145 | 0.01 |
| 2146 | 0.01 |
| 2147 | 0.01 |
| 2148 | 0.01 |
| 2149 | 0.02 |
| 2150 | 0.01 |
| 2151 | 0.02 |
| 2152 | 0.01 |
| 2153 | 0.02 |
| 2154 | 0.01 |
| 2155 | 0.01 |
| 2156 | 0.12 |
| 2157 | 0.04 |
| 2158 | 0.02 |
| 2159 | 5.2 nM |
| 2160 | 4.4 nM |
| 2161 | 0.04 |
| 2162 | 0.02 |
| 2163 | 0.01 |
| 2164 | 0.05 |
| 2165 | 0.04 |
| 2166 | 0.02 |
| 2167 | 0.05 |
| 2168 | 0.03 |
| 2169 | 0.01 |
| 2170 | 0.01 |
| 2171 | 0.15 |
| 2172 | 0.08 |
| 2173 | 0.19 |
| 2174 | 0.31 |
| 2175 | 0.23 |
| 2176 | 0.19 |
| 2177 | 0.03 |
| 2178 | 0.04 |
| 2179 | 0.06 |
| 2180 | 0.02 |
| 2181 | 0.05 |
| 2182 | 0.09 |
| 2183 | 0.11 |
| 2184 | 0.04 |
| 2185 | 6.1 nM |
| 2186 | 3.8 nM |
| 2187 | 9.8 nM |
| 2188 | 0.02 |
| 2189 | 0.03 |
| 2190 | 0.03 |
| 2191 | 0.03 |
| 2192 | 0.02 |
| 2193 | 0.01 |
| 2194 | 0.26 |
| 2195 | 0.08 |
| 2196 | 0.02 |
| 2197 | 2.8 nM |
| 2198 | 0.11 |
| 2199 | 2.4 nM |
| 2200 | 0.01 |
| 2201 | 0.01 |
| 2202 | 0.50 |
| 2203 | 0.01 |
| 2204 | 0.01 |
| 2205 | 0.06 |
| 2206 | 0.04 |
| 2207 | 0.01 |
| 2208 | 0.01 |
| 2209 | 0.01 |
| 2210 | 0.02 |
| 2211 | 0.08 |
| 2212 | 4.8 nM |
| 2213 | 0.02 |
| 2214 | 0.02 |
| 2215 | 0.03 |
| 2216 | 0.12 |
| 2217 | 0.01 |
| 2218 | 0.01 |
| 2219 | 0.02 |
| 2220 | 0.04 |
| 2221 | 0.06 |
| 2222 | 0.29 |
| 2223 | 0.04 |
| 2224 | 0.24 |
| 2225 | 0.08 |
| 2226 | 0.06 |
| 2227 | 0.81 |
| 2228 | 0.12 |
| 2229 | 0.11 |
| 2230 | 0.03 |
| 2231 | 0.02 |
| 2232 | 0.01 |
| 2233 | 0.02 |
| 2234 | 0.02 |
| 2235 | 0.30 |
| 2236 | 0.31 |
| 2237 | 0.01 |
| 2238 | 0.01 |
| 2239 | 0.02 |
| 2240 | 0.04 |
| 2241 | 0.81 |
| 2242 | 0.03 |
| 2243 | 0.02 |
| 2244 | 0.04 |
| 2245 | 0.01 |
| 2246 | 0.03 |
| 2247 | 0.06 |
| 2248 | 0.04 |
| 2249 | 0.08 |
| 2250 | 0.02 |
| 2251 | 0.02 |
| 2252 | 0.08 |
| 2253 | 0.04 |
| 2254 | 0.16 |
| 2255 | 0.06 |
| 2256 | 0.10 |
| 2257 | 0.06 |
| 2258 | 0.16 |
| 2259 | 0.10 |
| 2260 | 0.26 |
| 2261 | 0.01 |
| 2262 | 0.01 |
| 2263 | 0.01 |
| 2264 | 0.09 |
| 2265 | 0.02 |
| 2266 | 0.15 |
| 2267 | 2.2 nM |
| 2268 | 1.3 nM |
| 2269 | 0.40 nM |
| 2270 | 0.80 nM |
| 2271 | 1.10 nM |
| 2272 | 0.11 |
| 2273 | 0.40 nM |
| 2274 | 0.40 nM |
| 2275 | 0.20 |
| 2276 | 0.17 |
| 2277 | 0.23 |
| 2278 | 0.12 |
| 2279 | 0.11 |
| 2280 | 0.14 |
| 2281 | 0.77 |
| 2282 | — |
| 2283 | 0.15 |
| 2284 | 0.10 |
| 2285 | 7.8 nM |
| 2286 | 0.08 |
| 2287 | 4.1 nM |
| 2288 | 4.8 nM |
| 2289 | 1.3 nM |
| 2290 | 7.9 nM |
| 2291 | 0.21 |
| 2292 | 0.5 nM |
| 2293 | 2.6 nM |
| 2294 | 0.95 |
| 2295 | 3.1 nM |
| 2296 | 0.44 |
| 2297 | 2.5 nM |
| 2298 | 0.01 |
| 2299 | 2.0 nM |
| 2300 | 0.40 nM |
| 2301 | 0.98 |

### Jurkat-PD-1 Cell Binding High-Content Screening Assay (CBA): Method 2

Phycoerythrin (PE) was covalently linked to the Ig epitope tag of human PD-L1-Ig and fluorescently labeled PD-L1-Ig was used for binding studies with a Jurkat cell line over-expressing human PD-1 (Jurkat-PD-1). Briefly, 8×103 Jurkat-hPD-1 cells were seeded into 384 well plates in 20 μl of DMEM supplemented with 10% fetal calf serum. 100 nl of compound was added to cells followed by incubation at 37° C. for 2 hours. To interrogate potential issues with compound aggregation, compounds were tested in the presence and absence of tween-20 detergent. Briefly, compounds serially diluted in 100% DMSO was treated with 1.25% Tween-20 for 1 hour at room temperature before being added to cells. The final concentration of tween in the CBA was 0.0125%. Then, 5 μl of PE-labeled PD-L1-Ig (20 nM final), diluted in DMEM supplemented with 10% fetal calf serum. After 1 hour incubation, cells were fixed with 4% paraformaldehyde in dPBS containing 10 μg/ml Hoechst 33342 and then washed 3× in 100 μl dPBS. Data was collected and processed using a Cell Insight NXT High Content Imager and associated software.

### Jurkat-PD-1 Cell Binding High-Content Screening Assay (CBA): Method 3

Alexa fluor-647 was covalently linked to the Ig epitope tag of human PD-L1-Ig and fluorescently labeled PD-L1-Ig was used for the binding studies with a Jurkat cell line over-expressing human PD-1. Briefly, 2.5×10⁴ Jurkat-hPD-1 cells were seeded into 384 well plates in 20 μl of RPMI supplemented with 10% fetal calf serum. 12.5 nl of compound was added to cells followed by incubation at 37° C. for 2 hours. Then, 10 ul of Alexa fluor-647 labeled PD-L1-Ig (177 nM final), diluted in RPMI supplemented with 10% fetal calf serum was added to the cells and incubated for 1 hour. Cells were then fixed with 4% paraformaldehyde containing 10 μg/ml Hoechst 33342 and then washed 3× in 100 ul PBS and 100 ul of PBS was added in the final step. Data was collected using the Operetta High Content Imager and the data was analyzed using the columbus software.

### HTRF Assay

A time-resolved fluorescence resonance energy transfer (TR-FRET) assay was employed to measure the inhibition of the PD1-PDL1 protein-protein binding interaction. The reaction buffer was prepared by mixing the HiBlock buffer and LANCE detection buffer (PerkinElmer, TRF1011F and CR97-100) at a 1:1 ratio. Assay reactions contained 5 nM human programmed cell death 1 [hPD1(25-167)-hIgG, BMS], 5 nM human programmed cell death ligand 1 [hPD-L1 (19-239)-6×His, BMS], in the presence of 1 nM LANCE Eu-W1024 Anti-Human IgG (Eu-anti hIgG, PerkinElmer, AD0074) and 20 nM SureLight Allophycocyanin-anti-6×His antibody (APC-anti-6×His, PerkinElmer, AD0059H). Test compounds were evaluated in a 10-point dose-response format with serial three-fold dilutions starting from a 2 uM top concentration. Reactions were carried out in a total volume of 10 uL in 384-well microtiter plates (Proxiplate white, PerkinElmer, 6008289). Plates were sealed and incubated for 24 hours at 25° C. After 24 hours, the TR-FRET signal was measured using a PerkinElmer EnVision multimode plate reader and the resulting ratiometric data set (signal at 665 nm/615 nm, multiplied by a factor of 10,000) was recorded. The concentration giving half-maximal inhibition (the IC₅₀) was obtained by fitting the data to a four-parameter logistic equation using Graphpad Prism or Dotmatics.

TABLE 4

| | CBA | CBA | CBA | |
|---|---|---|---|---|
| | Method | Method | Method | HTRF |
| Example | 1 μM | 2 μM | 3 μM | μM |
| 5001 | 0.01 | | | |
| 5002 | 0.89 | | | |
| 5003 | 0.03 | | | |
| 5004 | 0.03 | | | |
| 5005 | 0.02 | | | |
| 5006 | 0.02 | | | |
| 5007 | 0.01 | | | |
| 5008 | 0.02 | | | |
| 5009 | 0.45 | | | |
| 5010 | 0.74 | | | |
| 5011 | 0.75 | | | |
| 5012 | 0.69 | | | |
| 5013 | 11 nM | 4.0 nM | | |
| 5014 | 0.17 | 0.04 | | |
| 5015 | 0.21 | 0.31 | | |
| 5016 | 0.05 | 0.050 | | |
| 5017 | 3.3 nM | 3.0 nM | | |
| 5018 | 3.1 nM | 2.9 nM | | |
| 5019 | 4.9 nM | 7.6 nM | | |
| 5050 | 5.8 nM | 5.0 nM | | |
| 5021 | 3.3 nM | 3.1 nM | | |
| 5022 | 13 nM | 8.3 nM | | |
| 5023 | 7.1 nM | 4.1 nM | | |
| 5024 | 8.5 nM | 6.7 nM | | |
| 5025 | 0.31 | 0.56 | | |
| 5026 | 0.12 | 0.12 | | |
| 5027 | 2.2 nM | 0.01 | | |
| 5028 | 0.0057 | 0.0044 | | |
| 5029 | 0.3559 | 0.2994 | | |
| 5030 | 0.0047 | 0.0043 | | |
| 5031 | 0.0087 | 0.0068 | | |
| 5032 | 0.0987 | 0.1077 | | |
| 5033 | 0.0032 | 0.0033 | | |
| 5034 | 0.0051 | 0.0031 | | |
| 5035 | 0.0042 | 0.0035 | | |
| 5036 | 0.0045 | 0.0031 | | |
| 5037 | 0.0138 | 0.0304 | | |
| 5038 | 0.0425 | 0.0171 | | |
| 5039 | 0.0729 | 0.0607 | | |
| 5040 | 0.0159 | 0.0146 | | |
| 5041 | 0.0888 | 0.0822 | | |
| 5042 | 0.0639 | 0.0689 | | |
| 5043 | 0.0098 | 0.0098 | | |
| 5044 | 0.1310 | 1.0000 | | |
| 5045 | 0.0016 | 0.0014 | | |
| 5046 | 0.5139 | 0.5170 | | |
| 5047 | 0.0052 | 0.0041 | | |
| 5048 | 0.0048 | 0.0044 | | |
| 5049 | 0.0083 | 0.0070 | | |
| 5050 | 0.0065 | 0.0059 | | |
| 5051 | 0.0032 | 0.0021 | | |
| 5052 | 0.0081 | 0.0084 | | |
| 5053 | 0.0090 | 0.0074 | | |
| 5054 | 0.0053 | 0.0057 | | |
| 5055 | 0.0385 | 0.0321 | | |
| 5056 | 0.0100 | 0.0095 | | |
| 5057 | 0.0089 | 0.0106 | | |
| 5058 | 0.0032 | 0.0024 | | |
| 5059 | 0.0029 | 0.0033 | | |
| 5060 | 0.0068 | 0.0093 | | |
| 5061 | 0.0025 | 0.0019 | | |
| 5062 | 0.0045 | 0.0040 | | |
| 5063 | 0.0054 | 0.0052 | | |
| 5064 | 0.0544 | 0.0348 | | |
| 5065 | 0.0077 | 0.0060 | | |
| 5066 | 0.0304 | 0.0028 | | |
| 5067 | 0.0072 | 0.0069 | | |
| 5068 | 0.0172 | 0.0106 | | |
| 5069 | 0.0136 | 0.0131 | | |
| 5070 | 0.0055 | 0.0061 | | |
| 5071 | 0.0322 | 0.0261 | | |
| 5072 | 0.0167 | 0.0140 | | |
| 5073 | 0.0059 | 0.0050 | | |
| 5074 | 0.0401 | 0.0267 | | |
| 5075 | 0.1282 | 0.3717 | | |
| 5076 | 0.4633 | 0.1114 | | |
| 5077 | 0.3447 | 0.3380 | | |
| 5078 | 0.5327 | 0.2636 | | |
| 5079 | 0.1378 | 0.2290 | | |
| 5080 | 0.2241 | 0.3321 | | |
| 5081 | 0.1730 | 0.1125 | | |
| 5082 | 0.0479 | 0.1103 | | |
| 5083 | 0.3546 | 1.0000 | | |
| 5084 | 0.0243 | 0.0332 | | |
| 5085 | 0.0315 | 0.0100 | | |
| 5086 | 5.5 nM | 9.9 nM | | |
| 5087 | 0.03 | 0.11 | | |
| 5088 | 0.09 | 0.20 | | |
| 5089 | 0.01 | 0.11 | | |
| 5090 | 0.01 | 8.7 nM | | |
| 5091 | 0.09 | 0.07 | | |
| 5092 | | | 0.10 | |
| 5093 | | | 0.16 | |
| 5094 | | | 0.03 | |
| 5095 | | | 6.1 nM | |
| 5096 | | | 0.04 | |
| 5097 | | | 0.02 | |
| 5098 | | | 0.01 | |
| 5099 | | | 0.65 | |
| 5100 | | | 0.27 | |
| 5101 | | | 3.7 nM | |
| 5102 | | | 0.51 | |
| 5103 | | | 0.38 | |
| 5104 | | | 0.05 | |
| 5105 | | | 0.02 | |
| 5106 | | | 0.06 | |
| 5107 | | | 0.33 | |
| 5108 | | | 7.3 nM | |
| 5109 | | | 4.4 nM | |
| 5110 | | | 8.8 nM | |
| 5111 | | | 4.7 nM | |
| 5112 | | | 0.10 | |
| 5113 | | | 0.15 | |
| 5114 | | | 0.11 | |
| 5115 | | | 0.28 | |
| 5116 | | | 0.33 | |
| 5117 | | | 9.7 nM | |
| 5118 | | | 1.7 nM | |
| 5119 | | | 0.02 | |
| 5120 | | | 5.6 nM | |
| 5121 | | | 0.07 | |
| 5122 | | | 0.11 | |
| 5123 | | | 9.4 nM | |
| 5124 | | | 2.6 nM | |
| 5125 | | | 9.4 nM | |
| 5126 | | | 3.2 nM | |
| 5127 | | | 0.02 | |
| 5128 | | | 0.03 | |
| 5129 | | | 0.03 | |
| 5130 | | | 0.06 | |
| 5131 | | | 0.19 | |
| 5132 | | | 0.02 | |
| 5133 | | | 0.20 | |
| 5134 | | | 0.03 | |
| 5135 | | | 6.8 nM | |
| 5136 | | | 0.02 | |
| 5137 | | | 4.4 nM | |
| 5138 | | | 0.03 | |
| 5139 | | | 0.03 | |
| 5140 | | | 0.01 | |
| 5141 | | | 0.02 | |
| 5142 | | | 3.3 nM | |
| 5143 | | | 7.0 nM | |
| 5144 | | | 9.7 nM | |
| 5145 | | | 0.9 nM | |
| 5146 | | | 0.09 | 0.03 |
| 5147 | | | 0.01 | |
| 5148 | | | 0.9 nM | |
| 5149 | | | 5.9 nM | |
| 5150 | | | 0.06 | |
| 5151 | | | 0.5 nM | |
| 5152 | | | 0.5 nM | |
| 5153 | | | 0.4 nM | |
| 5154 | | | 6.3 nM | |
| 5155 | | | 0.10 | |
| 5156 | | | 0.07 | |
| 5157 | | | 0.12 | |
| 5158 | | | 0.02 | |
| 5159 | | | 0.04 | |
| 5160 | | | 0.04 | |
| 5161 | | | 2.4 nM | |
| 5162 | | | 0.5 nM | |
| 5163 | | | 2.5 nM | |
| 5164 | | | 0.15 | |
| 5165 | | | 0.03 | |
| 5166 | | | 1.0 nM | |
| 5167 | | | 0.3 nM | |
| 5168 | | | 1.4 nM | |
| 5169 | | | 8.9 nM | |
| 5170 | | | 5.9 nM | |
| 5171 | | | 4.4 nM | |
| 5172 | | | 0.33 | |
| 5173 | | | 0.02 | 0.07 |
| 5174 | | | 0.01 | |
| 5175 | | | 0.01 | |
| 5176 | | | 0.01 | |
| 5177 | | | 7.6 nM | 0.03 |
| 5178 | | | 0.30 | |
| 5179 | | | 3.9 nM | |
| 5180 | | | 6.8 nM | |
| 5181 | | | 3.8 nM | |
| 5182 | | | 0.01 | |
| 5183 | | | 2.3 nM | |
| 5184 | | | 1.1 nM | |
| 5185 | | | 2.3 nM | 0.01 |
| 5186 | | | 4.5 nM | 0.03 |
| 5187 | | | 8.0 nM | |
| 5188 | | | 0.05 | |
| 5189 | | | 0.33 | |
| 5190 | | | 0.02 | |
| 5191 | | | 0.12 | |
| 5192 | | | 0.15 | |
| 5193 | | | 0.38 | |
| 5194 | | | 0.04 | |
| 5195 | | | 1.7 nM | |
| 5196 | | | 7.2 nM | |
| 5197 | | | 0.45 | |
| 5198 | | | 0.01 | |
| 5199 | | | 0.01 | |
| 5200 | | | 0.02 | |
| 5201 | | | 6.2 nM | |
| 5202 | | | 4.9 nM | |
| 5203 | | | 0.01 | |
| 5204 | | | 6.5 nM | |
| 5205 | | | 0.16 | |
| 5206 | | | 0.01 | 0.05 |
| 5207 | | | 0.03 | |
| 5208 | | | 3.8 nM | |
| 5209 | | | 0.02 | |
| 5210 | | | 0.14 | |
| 5211 | | | 0.01 | 0.04 |
| 5212 | | | 3.9 nM | 0.02 |
| 5213 | | | 0.33 | |
| 5214 | | | 0.03 | |
| 5215 | | | 1.0 nM | |
| 5216 | | | 6.0 nM | |
| 5217 | | | 0.26 | |
| 5218 | | | 0.02 | |
| 5219 | | | 0.10 | |
| 5220 | | | 2.4 nM | |
| 5221 | | | 3.0 nM | 0.01 |
| 5222 | | | 0.02 | |
| 5223 | | | 0.05 | |
| 5224 | | | 3.5 nM | |
| 5225 | | | 0.28 | |
| 5226 | | | 0.07 | |
| 5227 | | | 1.5 nM | |
| 5228 | | | 0.8 nM | |
| 5229 | | | 3.8 nM | 0.04 |
| 5230 | | | 0.03 | |
| 5231 | | | 0.14 | |
| 5232 | | | 0.10 | |
| 5233 | | | 0.02 | |
| 5234 | | | 3.4 nM | |
| 5235 | | | 3.6 nM | 0.03 |
| 5236 | | | 1.1 nM | 0.10 |
| 5237 | | | 4.4 nM | 0.03 |
| 5238 | | | 0.01 | |
| 5239 | | | 0.02 | 0.02 |
| 5240 | | | 4.4 nM | |
| 5241 | | | 0.4 nM | |
| 5242 | | | 2.1 nM | |
| 5243 | | | 2.2 nM | 0.03 |
| 5244 | | | 6.4 nM | 0.03 |
| 5245 | | | 0.01 | 0.01 |
| 5246 | | | 0.42 | |
| 5247 | | | 1.7 nM | |
| 5248 | | | 0.6 nM | |
| 5249 | | | 1.9 nM | |
| 5250 | | | 1.4 nM | |
| 5251 | | | 1.6 nM | |
| 5252 | | | 1.2 nM | |
| 5253 | | | 2.8 nM | |
| 5254 | | | 1.3 nM | |
| 5255 | | | 0.0022 | |
| 5256 | | | 3.4 nM | |
| 5257 | | | 0.03 | |
| 5258 | | | 0.6 nM | |
| 5259 | | | 0.01 | |
| 5260 | | | 0.09 | |
| 5261 | | | 0.06 | |
| 5262 | | | 0.11 | |
| 5263 | | | 0.18 | |
| 5264 | | | 0.10 | |
| 5265 | | | 0.03 | |
| 5266 | | | 2.0 nM | |
| 5267 | | | 1.2 nM | |
| 5268 | | | 5.3 nM | |
| 5269 | | | 0.02 | |
| 5270 | | | 0.02 | 0.0093 |
| 5271 | | | 1.0 nM | |
| 5272 | | | 0.01 | |
| 5273 | | | 0.5 nM | |
| 5274 | | | 6.0 nM | |
| 5275 | | | 1.6 nM | |
| 5276 | | | 1.1 nM | |
| 5277 | | | 0.03 | |
| 5278 | | | 0.31 | |
| 5279 | | | 3.0 nM | 0.0150 |
| 5280 | | | 4.6 nM | |
| 5281 | | | 0.16 | |
| 5282 | | | 0.17 | |
| 5283 | | | 0.01 | |
| 5284 | | | 0.20 | 0.01 |
| 5285 | | | 0.30 | |
| 5286 | | | 1.8 nM | |
| 5287 | | | 0.27 | |
| 5288 | | | 1.0 nM | 0.08 |
| 5289 | | | 0.8 nM | |
| 5290 | | | 0.10 | |
| 5291 | | | 0.2 nM | |
| 5292 | | | 0.2 nM | 0.03 |
| 5293 | | | 0.06 | |
| 5294 | | | 0.04 | |
| 5295 | | | 1.6 nM | |
| 5296 | | | 0.02 | |
| 5297 | | | | 2.3 nM |
| 5298 | | | 0.5 nM | 0.01 |

It is to be appreciated that the Detailed Description section, and not the Summary and Abstract sections, is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A compound of Formula (I):
or a pharmaceutically acceptable salt thereof, wherein:
R¹ is selected from C₁-C₃alkoxyC₁-C₃alkyl; C₁-C₆alkyl; C₁-C₃alkylS(O)C₁-C₆alkyl;
mono-, di- or tri-C₁-C₆alkylaminoC₁-C₆alkyl; aminoC₁-C₆alkyl; aminocarbonylC₁-C₆alkyl;
arylC₁-C₆alkyl; carbamidylC₁-C₆alkyl; carboxyC₁-C₃alkyl; cyanoC₁-C₆alkyl;
C₃-C₆cycloalkylC₁-C₆alkyl; C₃-C₆cycloalkylcarbonylaminoC₁-C₆alkyl; heteroarylC₁-C₆alkyl;
heterocyclylC₁-C₆alkyl; hydroxyC₁-C₆alkyl; H₂NC(X)NHC₁-C₆alkyl; and H₂NC(X) , where X is O or NH, and represents an azetidine, pyrrolidine, or piperidine ring; and wherein the aryl part of the arylC₁-C₆alkyl and the heteroaryl part of the heteroarylC₁-C₆alkyl are optionally substituted with one, two, three, four, or five groups independently selected from aminoC₂-C₆alkoxy, C₁-C₃alkyl, C₁-C₃alkylcarbonylaminoC₁-C₃alkyl, aminoC₁-C₆alkyl, R⁷⁰NHC₁-C₆alkyl, aminocarbonyl, carboxy, carboxyC₁-C₆alkoxy, carboxyC₁-C₆alkyl, guanidinylC₁-C₆alkyl, halo, haloC₁-C₃alkyl, hydroxy, nitro, and phenyl optionally substituted with a C₁-C₃alkylcarbonylamino or a carboxy group; wherein R⁷⁰ is selected from C₁-C₃alkylcarbonyl, arylC₁-C₃alkylcarbonyl, C₃-C₆cycloalkylcarbonyl, and heteroarylC₁-C₃alkylcarbonyl;
R² is selected from C₂-C₆alkenyl; C₁-C₆alkylcarbonylaminoC₁-C₆alkylthioC₁-C₆alkyl; aminocarbonylC₁-C₆alkyl; arylC₁-C₆alkyl; aryl-arylC₁-C₃alkyl; aryl-heteroarylC₁-C₃alkyl; heteroarylC₁-C₆alkyl; and hydroxyC₁-C₆alkyl; wherein the aryl part of the arylC₁-C₆alkyl and the aryl-arylC₁-C₃alkyl is optionally substituted with one, two, three, four, or five groups independently selected from C₂-C₆alkenyl, C₂-C₆alkenyloxy, C₁-C₆alkoxy, C₁-C₆alkyl, C₁-C₆alkylcarbonyloxyC₁-C₆alkoxy, C₂-C₆alkynyloxy, amino, aminoC₁-C₆alkoxy, aminoC₁-C₆alkyl, aminocarbonyl, aryloxy, carboxy, carboxyC₁-C₆alkoxy, cyano, halo, hydroxy, hydroxyC₂-C₆alkenyl, carboxyaryl, nitro, trifluoromethyl, and —OP(O)X¹X², wherein each of X¹ and X² is —OH, —NH₂, or —N(C₁-C₆alkyl)₂;
R³ is selected from aminocarbonylC₁-C₃alkyl; C₁-C₃alkylsulfonylaminocarbonylC₁-C₃alkyl; arylsulfonylaminocarbonylC₁-C₃alkyl; bis(carboxyC₁-C₃alkyl)aminoC₁-C₃alkylcarbonylaminoC₁-C₃alkyl; carboxyC₁-C₃alkyl; carboxyC₁-C₃alkylaminocarbonylC₁-C₃alkyl; carboxyC₁-C₃alkylcarbonylaminoC₁-C₃alkyl; dimethylaminosulfonylaminocarbonylC₁-C₃alkyl; heteroarylaminocarbonylC₁-C₃alkyl, (OH)₂P(O)OC₁-C₃alkyl; tetrazolylC₁-C₃alkyl; and R⁶⁵R⁶⁶C═C(CH₃)—NHC₁-C₃alkyl; wherein R⁶⁵ and R⁶⁶, together with the carbon atom to which they are attached, form a five- to seven-membered cycloalkyl ring optionally substituted with one, two, three, or four groups selected from C₁-C₃alkyl and oxo; wherein the aryl part of the arylsulfonylaminocarbonylC₁-C₃alkyl is optionally substituted with one, two, or three groups selected from C₁-C₃alkoxycarbonyl and halo;
R⁴ is selected from arylC₁-C₆alkyl and heteroarylC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, cyano, fluoroC₁-C₆alkyl, and halo;
R⁵ is selected from C₂-C₆alkenyl; C₁-C₆alkyl; C₁-C₆alkylcarbonylaminoC₁-C₆alkylthioC₁-C₆alkyl; aminoC₁-C₆alkyl; aminocarbonylC₁-C₆alkyl; arylC₁-C₆alkyl; aryl-arylC₁-C₃alkyl; arylcarbonylaminoC₁-C₃alkylarylC₁-C₃alkyl; carboxyC₁-C₆alkyl; cyanoC₁-C₆alkyl; C₃-C₈cycloalkyl; (C₃-C₈cycloalkyl)C₁-C₆alkyl; (C₃-C₈cycloalkyl)carbonylaminoC₁-C₃alkylarylC₁-C₃alkyl; and heteroarylC₁-C₆alkyl; heteroaryl-arylC₁-C₃alkyl, heteroarylcarbonylaminoC₁-C₃alkylarylC₁-C₃alkyl and hydroxyC₁-C₆alkyl; wherein the aryl part of the arylC₁-C₆alkyl, the aryl-arylC₁-C₃alkyl, and the arylcarbonylaminoC₁-C₃alkylarylC₁-C₃alkyl, and the heteroaryl part of the heteroarylC₁-C₆alkyl and the heteroaryl-arylC₁-C₃alkyl are optionally substituted with one, two, three, four, or five groups independently selected from C₁-C₆alkyl, C₁-C₃alkylcarbonylamino, amino, aminoC₁-C₆alkyl, aminocarbonyl, C₁-C₃alkylaminosulfonyl, carboxy, carboxyC₁-C₆alkoxy, cyano, C₃-C₈cycloalkyl, (C₃-C₈cycloalkyl)oxy, fluoroC₁-C₆alkyl, halo, haloC₁-C₃alkyl, hydroxy, heterocyclylsulfonyl, and phenylcarbonyl;
R⁶ is aryl-arylC₁-C₃alkyl, heteroaryl-arylC₁-C₃alkyl, aryl-heteroarylC₁-C₃alkyl, heteroaryl-heteroarylC₁-C₃alkyl, wherein the aryl or the heteroaryl part is optionally substituted with one, two, three, four, or five groups independently selected from C₁-C₆alkylcarbonylamino, aminocarbonyl, fluoroC₁-C₆alkyl, halo, hydroxy, trifluoromethoxy, C₁-C₆alkoxy, C₁-C₆alkoxyC₁-C₆alkyl, carboxyC₁-C₆alkoxyC₁-C₆alkyl, cyanoC₁-C₆alkyl, and arylC₁-C₆alkoxy;
R⁷ is selected from hydrogen; C₂-C₆alkenyl; C₁-C₆alkyl; C₁-C₃alkylcarbonylaminoC₁-C₆alkyl; aminoC₁-C₆alkyl; aminocarbonylC₁-C₆alkyl; carboxyC₁-C₆alkyl; arylC₁-C₆alkyl; aryl-arylC₁-C₃alkyl; C₁-C₆alkylcarbonylaminoC₁-C₆alkylthioC₁-C₆alkyl; C₁-C₆alkylcarbonylaminoC₁-C₆alkyl; guanidinyl; heteroarylC₁-C₆alkyl; hydroxyC₁-C₆alkyl; and NH₂C(X)NHC₁-C₆alkyl, where X is O or NH; and wherein the aryl part of the arylC₁-C₆alkyl and the aryl-arylC₁-C₃alkyl, and the heteroaryl part of the heteroarylC₁-C₆alkyl are optionally substituted with one, two, three, four, or five groups independently selected from aminoC₁-C₆alkyl, aminocarbonyl, carboxy, carboxyC₁-C₆alkoxy, and hydroxy;
R⁸ is selected from C₁-C₆alkyl; aminoC₁-C₆alkyl; carboxyC₁-C₆alkyl; aryl; arylC₁-C₆alkyl; heteroarylC₁-C₆alkyl; and hydroxyC₁-C₆alkyl; wherein the aryl part of the arylC₁-C₆alkyl is optionally substituted with one, two, three, four, or five groups independently selected from halo and hydroxy;
R⁹ is selected from hydrogen; C₁-C₆alkyl; aminoC₁-C₆alkyl; aminocarbonylC₁-C₆alkyl; aryl; arylC₁-C₆alkyl; carboxyC₁-C₆alkyl; C₃-C₈cycloalkyl; C₃-C₈cycloalkylC₁-C₆alkyl; heteroarylC₁-C₆alkyl; hydroxyC₁-C₆alkyl; C₁-C₆alkylthioC₁-C₆alkyl; and NH₂C(X)NHC₁-C₆alkyl, where X is O or NH; and wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amino, carboxyC₁-C₆alkyl, cyano, halo, hydroxy, nitro, and trifluoromethyl;
R¹⁰ is selected from C₁-C₆alkyl; C₁-C₆alkylcarbonylaminoC₁-C₆alkyl; C₁-C₆alkylNHC₁-C₆alkyl; aminoC₁-C₆alkyl; aminocarbonylC₁-C₆alkyl; carboxyC₁-C₆alkyl; hydroxyC₁-C₆alkyl;
NH₂C(X)NHC₁-C₆alkyl, where X is O or NH; heteroarylC₁-C₆alkyl; and arylC₁-C₆alkyl; and
wherein the aryl part of the arylC₁-C₆alkyl is optionally substituted with one, two, three, four, or five aminoC₁-C₆alkyl groups;
R¹¹ is selected from C₁-C₆alkyl, aminoC₁-C₆alkyl, arylC₁-C₆alkyl, C₃-C₈cycloalkylC₁-C₆alkyl, heteroarylC₁-C₆alkyl, and heterocyclylC₁-C₆alkyl; wherein the aryl part of the arylC₁-C₆alkyl, the heteroaryl part of the heteroarylC₁-C₆alkyl, and the heterocyclyl part of the heterocyclylC₁-C₆alkyl are optionally substituted with one, two, three, four, or five groups independently selected from C₁-C₆alkoxy, C₁-C₆alkyl, amino, aminoC₁-C₃alkyl, halo, and hydroxy;
R¹² is selected from C₂-C₆alkenyl; C₁-C₆alkyl; C₁-C₆alkylcarbonylaminoC₁-C₆alkylthioC₁-C₆alkyl; aminoC₁-C₆alkyl; arylC₁-C₆alkyl; carboxyC₁-C₆alkyl; hydroxyC₁-C₆alkyl; and NH₂C(X)NHC₁-C₆alkyl, where X is O or NH;
R¹³ is selected from C₂-C₆alkenyl; C₁-C₆alkyl; C₁-C₆alkylcarbonylaminoC₁-C₆alkyl; C₁-C₆alkylcarbonylaminoC₁-C₆alkylthioC₁-C₆alkyl; aminoC₁-C₆alkyl; aminocarbonylC₁-C₆alkyl; carboxyC₁-C₆alkyl; carboxyC₁-C₆alkylcarbonylaminoC₁-C₃alkyl; cyanoC₁-C₆alkyl;
C₃-C₈cycloalkyl; heteroarylC₁-C₆alkyl; hydroxyC₁-C₆alkyl; haloC₁-C₆alkylcarbonylaminoC₁-C₃alkyl; hydroxyC₁-C₆alkylcarbonylaminoC₁-C₃alkyl; and NH₂C(X)NHC₁-C₆alkyl, where X is O or NH;
R¹⁴ is aminocarbonyl or —C(O)NR^{14′}CR¹⁵R^{15′}R^{15″}, wherein
R^{14′} is hydrogen, or R¹⁵ and R^{14′}, together with the atoms to which they are attached, form an azetidine, morpholine, piperidine, piperazine, or pyrrolidine ring, wherein each ring is optionally substituted with an amino, aminocarbonyl, or a hydroxy group;
R¹⁵ is selected from hydrogen; C₂-C₆alkenyl; C₁-C₆alkyl; C₁-C₆alkylcarbonylaminoC₁-C₆alkyl; C₁-C₆alkylcarbonylaminoC₁-C₆alkylthioC₁-C₆alkyl; aminoC₁-C₆alkyl; aminocarbonylC₁-C₆alkyl; carboxy; carboxyC₁-C₆alkyl; heterocyclyl; hydroxyC₁-C₆alkyl; and NH₂C(X)NHC₁-C₆alkyl, where X is O or NH;
R^{15′} is hydrogen, or R¹⁵ and R^{15′}, together with the atoms to which they are attached, form a C₃-C₈cycloalkyl ring; and
R^{15″} is hydrogen; —C(O)NH₂, or —(CH₂)ₙC(O)NHCHR¹⁶R^{16′}; wherein
n is 0, 1, or 2;
R¹⁶ is selected from hydrogen, C₂-C₆alkynyl, aminoC₁-C₆alkyl, carboxyC₁-C₆alkyl, and hydroxyC₁-C₃alkyl;
R^{16′} is hydrogen; C₁-C₆alkyl; aminocarbonyl; carboxy; or —C(O)NHCHR¹⁷R^{17′}; wherein
R¹⁷ is hydrogen or hydroxyC₁-C₃alkyl; and
R^{17′} is —C(O)NH₂ or —C(O)NHCHR¹⁸R^{18′}; wherein
R¹⁸ is aminoC₁-C₆alkyl; and
R^{18′} is carboxy.

2. The compound of claim 1, or the pharmaceutically acceptable salt thereof, wherein R¹ is selected from aminoC₁-C₆alkyl; aminocarbonylC₁-C₆alkyl; arylC₁-C₆alkyl; C₃-C₆cycloalkylC₁-C₆alkyl; heteroarylC₁-C₆alkyl; and hydroxyC₁-C₆alkyl; wherein the aryl part of the arylC₁-C₆alkyl is optionally substituted with one, two, or three groups independently selected from C₁-C₃alkyl, carboxyC₁-C₆alkoxy, halo, and haloC₁-C₃alkyl.

3. The compound of claim 1, or the pharmaceutically acceptable salt thereof, wherein R² is selected from aryl-arylC₁-C₂alkyl, arylC₁-C₆alkyl and heteroarylC₁-C₆alkyl, wherein the aryl part of the aryl-arylC₁-C₂alkyl and the arylC₁-C₆alkyl are optionally substituted with one, two, or three groups independently selected from carboxy, carboxyC₁-C₆alkoxy, cyano, halo, and hydroxy.

4. The compound of claim 1, or the pharmaceutically acceptable salt thereof, wherein R³ is aminocarbonylC₁-C₃alkyl, carboxyC₁-C₃alkyl, or tetrazolylC₁-C₆alkyl.

5. The compound of claim 1, or the pharmaceutically acceptable salt thereof, wherein R⁴ is arylC₁-C₃alkyl or heteroarylC₁-C₃alkyl, wherein the aryl part of the arylC₁-C₃alkyl and the heteroaryl part of the heteroarylC₁-C₆alkyl are optionally substituted with one, two, three, four, or five groups independently selected from C₁-C₆alkyl and cyano.

6. The compound of claim 1, or the pharmaceutically acceptable salt thereof, wherein R⁵ is C₁-C₆alkyl, aryl-arylC₁-C₃alkyl, or arylC₁-C₆alkyl, wherein the aryl part of the arylC₁-C₆alkyl is optionally substituted with one, two, or three groups independently selected from carboxy, carboxyC₁-C₆alkoxy, hydroxy, and methylcarbonylamino.

7. The compound of claim 1, or the pharmaceutically acceptable salt thereof, wherein R⁶ is aryl-arylC₁-C₆alkyl.

8. The compound of claim 1, or the pharmaceutically acceptable salt thereof, wherein R⁷ is selected from C₁-C₆alkyl; and arylC₁-C₆alkyl; carboxyC₁-C₆alkyl; and NH₂C(X)NHC₁-C₆alkyl, where X is O or NH; and wherein the aryl part of the arylC₁-C₆alkyl is optionally substituted with one, two, or three groups independently selected from carboxy, carboxyC₁-C₆alkoxy and hydroxy.

9. The compound of claim 1, or the pharmaceutically acceptable salt thereof, wherein R⁸ is C₁-C₆alkyl.

10. The compound of claim 1, or the pharmaceutically acceptable salt thereof, wherein
R⁹ is C₁-C₆alkyl or arylC₁-C₆alkyl; and
R^{9′} is hydrogen or methyl.

11. The compound of claim 1, or the pharmaceutically acceptable salt thereof, wherein R¹⁰ is aminoC₁-C₆alkyl; aminocarbonylC₁-C₆alkyl; or NH₂C(X)NHC₁-C₆alkyl, where X is O or NH.

12. The compound of claim 1, or the pharmaceutically acceptable salt thereof, wherein R¹¹ is C₁-C₄alkyl or C₃-C₆cycloalkylC₁-C₃alkyl.

13. The compound of claim 1, or the pharmaceutically acceptable salt thereof, wherein R¹² is C₁-C₄alkyl or hydroxyC₁-C₄alkyl.

14. The compound of claim 1, or the pharmaceutically acceptable salt thereof, wherein R¹³ is aminoC₁-C₆alkyl, carboxyC₁-C₆alkyl, or hydroxyC₁-C₄alkyl.

15. The compound of claim 1, or the pharmaceutically acceptable salt thereof, wherein R¹⁴ is aminocarbonyl or —C(O)NHCHR¹⁵C(O)NH₂; and wherein R¹⁵ is hydrogen or C₁-C₆alkyl.

16. The compound of claim 1, or the pharmaceutically acceptable salt thereof, wherein R¹⁵ is hydrogen; C₁-C₆alkyl; aminoC₁-C₆alkyl; or carboxyC₁-C₆alkyl.

17. The compound of claim 1, or the pharmaceutically acceptable salt thereof, wherein R¹⁶ is hydrogen or C₂-C₄alkynyl.

18. The compound of claim 1, or the pharmaceutically acceptable salt thereof, wherein
R¹ is selected from aminoC₁-C₄alkyl; aminocarbonylC₁-C₃alkyl; butyl; carbamidylC₃-C₄alkyl; cyanoC₁-C₆alkyl; cyclohexylmethyl; cyclopropylcarbonylaminopropyl; guanidinylC₃-C₄alkyl; heteroarylC₁-C₂alkyl; heterocyclylmethyl; hydroxyethyl; mono-, di-, or tri-methylaminoC₁-C₆alkyl; and H₂NC(X) , where X is O or NH, and represents a piperidine ring; arylC₁-C₂alkyl; wherein the aryl part of the arylC₁-C₂alkyl is optionally substituted with one, two, or three groups independently selected from C₁-C₃alkyl, aminocarbonyl, aminoethoxy, aminomethyl, carboxy, carboxymethoxy, halo, haloC₁-C₃alkyl, hydroxy, and nitro;
R² is selected from aryl-arylC₁-C₂alkyl; arylC₁-C₂alkyl; hydroxyethyl; heteroarylC₁-C₂alkyl; methylcarbonylaminomethylthiomethyl; and propenyl; wherein the aryl part of the aryl-arylC₁-C₂alkyl and the arylC₁-C₂alkyl are optionally substituted with one, two, or three groups independently selected from amino, aminocarbonyl, aminoethoxy, aminomethyl, aryloxy, carboxy, carboxymethoxy, cyano, halo, hydroxy, methyl, methoxy, nitro, propenoxyl, propenyl, propynoxyl, trifluoromethyl, and —OP(O)X¹X², wherein each of X¹ and X² independently is amino, hydroxy, or mono- or di-methylamino;
R³ is selected from aminocarbonylmethyl; carboxymethyl; methyl dihydrogen phosphate; and tetrazolylmethyl;
R⁴ is selected from arylmethyl and heteroarylmethyl; and wherein the aryl part of the arylmethyl and the heteroaryl part of the heteroarylmethyl are optionally substituted with one, two, three, four, or five groups independently selected from cyano, halo, methyl, methoxy, and trifluoromethyl;
R⁵ is selected from C₃-C₄alkyl; aminocarbonylethyl; aminoethyl; arylmethyl; biphenylmethyl; carboxyethyl; cyanomethyl; cyclohexylmethyl; cyclopentyl; heteroarylmethyl; hydroxypropyl; methylcarbonylaminomethylthiomethyl; and propenyl; and wherein the distal phenyl of the biphenylmethyl and the aryl part of the arylmethyl is optionally substituted with one, two, or three groups independently selected from aminocarbonyl, aminomethyl, carboxy, carboxymethoxy, halo, hydroxy, methyl, and methylcarbonylamino;
R⁶ is aryl-arylmethyl, wherein the terminal aryl part of the aryl-arylmethyl is optionally substituted with one, two, or three groups independently selected from C₁-C₂alkoxy, aminocarbonyl, benzyloxy, carboxymethoxyC₁-C₂alkyl, cyanoethyl, halo, hydroxy, methoxymethyl, methylcarbonylaminotrifluoromethoxy, heteroaryl, and, trifluoromethyl;
R⁷ is selected from hydrogen; C₁-C₅alkyl; aminoC₃-C₄alkyl; aminocarbonylC₁-C₂alkyl; arylmethyl; carboxyC₁-C₃alkyl; heteroarylmethyl; hydroxyC₁-C₃alkyl; methylcarbonylaminomethylthiomethyl; methylcarbonylaminoC₃-C₄alkyl; propenyl; and NH₂C(X)NHpropyl, where X is O or NH; and wherein the aryl part of the arylmethyl is optionally substituted with one, two, or three groups independently selected from aminocarbonyl, aminoC₁-C₂alkyl, carboxy, carboxymethoxy, and hydroxy;
R⁸ is selected from C₁-C₄alkyl; aminopropyl; aryl; arylmethyl; carboxymethyl;
heteroarylmethyl; and hydroxymethyl; wherein the aryl part of the arylmethyl is optionally substituted with one, two, three, four, or five hydroxy groups;
R⁹ is selected from hydrogen; C₁-C₄alkyl; cyclohexyl; cyclohexylmethyl; aminoC₁-C₄alkyl; aminocarbonylC₁-C₆alkyl; carboxyC₁-C₆alkyl; aryl; arylmethyl; hydroxyC₁-C₂alkyl; heteroarylmethyl; methylthioethyl; and NH₂C(X)NHpropyl, where X is O or NH; and wherein the aryl part of the arylmethyl and the heteroaryl part of the heteroarylmethyl are optionally substituted with one or more groups independently selected from halo, trifluoromethyl, nitro, amino, cyano, methyl, methoxy, and carboxymethyl;
R^{9′} is hydrogen or methyl;
R¹⁰ is selected from C₁-C₃alkyl; aminoC₁-C₄alkyl; aminocarbonylmethyl; carboxyC₁-C₂alkyl; hydroxyethyl; C₁-C₄alkylcarbonylaminoethyl; methylaminoethyl; and NH₂C(X)NHpropyl, where X is O or NH; heteroarylmethyl; and arylmethyl; and wherein the aryl part of the arylmethyl is optionally substituted with one, two, or three aminomethyl;
R¹¹ is selected from C₂-C₄alkyl or C₃-C₆cycloalkylmethyl;
R¹² is selected from C₃-C₄alkyl; aminoC₁-C₄alkyl; arylmethyl; carboxyC₁-C₃alkyl; hydroxyC₂-C₃alkyl; methylcarbonylaminomethylthiomethyl; propenyl; and NH₂C(X)NHpropyl, where X is O or NH;
R¹³ is selected from aminoC₁-C₄alkyl; aminocarbonylC₁-C₂alkyl; butyl; carboxyC₁-C₂alkyl; cyanomethyl; cyclopentyl; heteroarylmethyl; hydroxyC₁-C₃alkyl; methylcarbonylaminobutyl; methylcarbonylaminomethylthiomethyl; propenyl; and NH₂C(X)NHpropyl, where X is O or NH;
R¹⁴ is aminocarbonyl or —C(O)NR^{14′}CR¹⁵R^{15′}R^{15″}, wherein
R^{14′} is hydrogen, or R¹⁵ and R^{14′}, together with the atoms to which they are attached, form a pyrrolidine ring;
R¹⁵ is selected from hydrogen; C₁-C₃alkyl; C₁-C₄alkylcarbonylaminoethyl; aminoC₁-C₄alkyl; aminocarbonylmethyl; carboxy; carboxyC₁-C₂alkyl; heterocyclyl; hydroxyC₁-C₃alkyl; methylcarbonylaminomethylthiomethyl; propenyl; and NH₂C(X)NHpropyl, where X is O or NH;
R^{15′} is hydrogen or R¹⁵ and R^{15′}, together with the atoms to which they are attached, form a cyclopropyl ring; and
R^{15″} is hydrogen; aminocarbonyl; carboxy; or —(CH₂)ₙC(O)NHCHR¹⁶R^{16′}; wherein
n is 0 or 1;
R¹⁶ is selected from hydrogen, C₃-C₄alkynyl, aminoC₁-C₅alkyl, and carboxyethyl; and
R^{16′} is hydrogen; C₁-C₂alkyl; aminocarbonyl; carboxy; or —C(O)NHCHR¹⁷R¹⁷;
wherein
R¹⁷ is hydrogen; and
R^{17′} is —C(O)CHR¹⁸R^{18″}; wherein
R¹⁸ is aminoethyl; and
R^{18′} is carboxy.

19. The compound of claim 18, or the pharmaceutically acceptable salt thereof, wherein R¹ is selected from aminoC₁-C₄alkyl; aminocarbonylC₁-C₃alkyl; arylC₁-C₂alkyl; cyclohexylmethyl; heteroarylmethyl; and hydroxyethyl; wherein the aryl part of the arylC₁-C₂alkyl is optionally substituted with one, two, or three groups independently selected from C₁-C₃alkyl, aminocarbonyl, halo, haloC₁-C₃alkyl, hydroxy, and nitro.

20. The compound of claim 1, or the pharmaceutically acceptable salt thereof, wherein
R¹ is selected from aminoC₁-C₄alkyl; butyl; aminocarbonylC₁-C₃alkyl; arylC₁-C₂alkyl; carbamidylC₃-C₄alkyl; cyanoC₁-C₆alkyl; cyclohexylmethyl; cyclopropylcarbonylaminopropyl; guanidinylC₃-C₄alkyl; heteroarylC₁-C₂alkyl; heterocyclylmethyl; hydroxyethyl; mono-, di-, or tri-methylaminoC₁-C₆alkyl; and H₂NC(X) , where X is O or NH, and represents a piperidine ring; wherein the aryl part of the arylC₁-C₂alkyl is optionally substituted with one, two, or three groups independently selected from C₁-C₃alkyl, halo, haloC₁-C₃alkyl, nitro, aminocarbonyl, aminomethyl, aminoethoxy, carboxy, or carboxymethoxy;
R² is selected from aryl-arylC₁-C₂alkyl, arylC₁-C₂alkyl; heteroarylC₁-C₂alkyl; hydroxyethyl; methylcarbonylaminomethylthiomethyl; and propenyl; wherein the aryl part of the aryl-arylC₁-C₂alkyl and the arylC₁-C₂alkyl are optionally substituted with one, two, or three groups independently selected from amino, aminocarbonyl, aminoethoxy, aminomethyl, carboxy, carboxymethoxy, cyano, halo, hydroxy, nitro, methoxy, methyl, propenyl, trifluoromethyl, and —OP(O)X¹X², wherein each of X¹ and X² independently is hydroxy, amino, or dimethylamino;
R³ is selected from aminocarbonylmethyl; carboxymethyl; and tetrazolylmethyl;
R⁴ is selected from arylmethyl and heteroarylmethyl; and wherein the aryl part of the arylmethyl and the heteroaryl part of the heteroarylmethyl are optionally substituted with one or more groups independently selected from bromo, chloro, cyano, methoxy, methyl, and trifluoromethyl;
R⁵ is selected from C₃-C₄alkyl; arylmethyl; biphenylmethyl; cyclopentyl; cyclohexylmethyl; hydroxypropyl; and propenyl; and wherein the distal phenyl of the biphenylmethyl and the aryl part of the arylmethyl is optionally substituted with one, two, or three groups independently selected from aminocarbonyl, carboxy, and carboxymethoxy, fluoro, hydroxy, and methylcarbonylamino;
R⁶ is aryl-arylmethyl; and wherein the terminal aryl part of the aryl-arylmethyl is optionally substituted with one, two, or three groups independently selected from chloro, fluoro, and thiophenyl;
R⁷ is selected from C₁-C₅alkyl; propenyl; aminoC₃-C₄alkyl; hydroxyC₁-C₃alkyl; aminocarbonylC₁-C₂alkyl; carboxyC₁-C₃alkyl; arylmethyl; heteroarylmethyl; methylcarbonylaminoC₃-C₄alkyl; and NH₂C(X)NHpropyl, where X is O or NH; and wherein the aryl part of the arylmethyl is optionally substituted with one, two, or three groups independently selected from hydroxy, aminocarbonyl, carboxy, aminoC₁-C₂alkyl, and carboxymethoxy;
R⁸ is selected from C₁-C₄alkyl; hydroxymethyl; phenyl; and phenylmethyl; wherein the phenyl part of the phenylmethyl is optionally substituted with one, two, or three hydroxy groups;
R⁹ is selected from hydrogen; C₁-C₄alkyl; aminocarbonylC₁-C₂alkyl; arylmethyl; cyclohexyl; cyclohexylmethyl; and heteroarylmethyl; and wherein the aryl part of the arylmethyl and the heteroaryl part of the heteroarylmethyl are optionally substituted with one, two, three, four, or five groups independently selected from carboxymethyl and cyano;
R^{9′} is hydrogen;
R¹⁰ is selected from C₁-C₄alkylcarbonylaminoethyl; aminoC₁-C₄alkyl;
aminocarbonylmethyl; arylmethyl; carboxyC₁-C₂alkyl; heteroarylmethyl; hydroxyethyl; methyl; methylaminoethyl; and NH₂C(X)NHpropyl, where X is O or NH; wherein the aryl part of the arylmethyl is optionally substituted with one, two, or three aminomethyl groups;
R¹¹ is selected from butyl; cyclohexylmethyl; cyclopropylmethyl; isobutyl; and isopentyl;
R¹² is selected from C₃-C₄alkyl; aminoC₃-C₄alkyl; carboxyC₁-C₃alkylisopropyl; carboxypropyl; hydroxyC₂-C₃alkyl; imidazolylmethyl; phenylmethyl; and propenyl;
R¹³ is selected from aminoC₁-C₄alkyl; aminocarbonylC₁-C₂alkyl; carboxyC₁-C₂alkyl; cyanomethyl; hydroxyC₁-C₂alkyl; methylcarbonylaminobutyl; propenyl; and NH₂C(X)NHpropyl, where X is O or NH, and
R¹⁴ is aminocarbonyl or —C(O)NR^{14′}CR¹⁵R^{15′}R^{15″}, wherein
R^{14′} is hydrogen, or R¹⁵ and R^{14′}, together with the atoms to which they are attached, form a pyrrolidine ring;
R¹⁵ is selected from hydrogen; aminoC₁-C₄alkyl; aminocarbonylmethyl; butylcarbonylaminoethyl; carboxy; carboxyC₁-C₂alkyl; hydroxymethyl; methyl; propenyl; methylcarbonylaminoethyl; methylcarbonylaminomethylthiomethyl; and NH₂C(X)NHpropyl, where X is O or NH;
R^{15′} is hydrogen or R¹⁵ and R^{15′}, together with the atoms to which they are attached, form a cyclopropyl ring; and
R^{15″} is hydrogen; aminocarbonyl; carboxy; or —(CH₂)ₙC(O)NHCHR¹⁶R^{16′}; wherein
R¹⁶ is selected from hydrogen; C₃-C₄alkynyl; aminoC₁-C₄alkyl; and carboxyethyl; and
R^{16′} is hydrogen; C₁-C₂alkyl; aminocarbonyl; carboxy; or —C(O)NHCHR¹⁷R¹⁷; wherein
n is 0 or 1;
R¹⁷ is hydrogen; and
R^{17′} is —C(O)CHR¹⁸R^{18′}; wherein
R¹⁸ is aminoethyl; and
R^{18′} is carboxy.

21. The compound of claim 20, or the pharmaceutically acceptable salt thereof, wherein
R¹ is selected from aminoC₁-C₄alkyl; aminocarbonylmethyl; arylC₁-C₂alkyl; carbamidylC₃-C₄alkyl; cyanomethyl; cyclohexylmethyl; cyclopropylcarbonylaminopropyl; guanidinylC₃-C₄alkyl; heteroarylC₁-C₂alkyl; heterocyclylmethyl; 1-hydroxyethyl; mono-, di-, or tri-methylaminoC₁-C₆alkyl; and H₂NC(X) , where X is O or NH, and represents a piperidine ring; wherein the aryl part of the arylC₁-C₂alkyl is optionally substituted with one, two, or three groups independently selected from aminocarbonyl, aminomethyl, aminoethoxy, carboxy, carboxymethoxy, methyl, fluoro, and trifluoromethyl;
R² is selected from aryl-arylC₁-C₂alkyl, arylC₁-C₂alkyl and heteroarylC₁-C₂alkyl; wherein the aryl part of the aryl-arylC₁-C₂alkyl and the arylC₁-C₂alkyl are optionally substituted with one, two, or three groups independently selected from aminocarbonyl, aminoethoxy, aminomethyl, carboxy, carboxymethoxy, cyano, fluoro, hydroxy, methoxy, methyl, nitro, and propenoxyl;
R³ is selected from aminocarbonylmethyl; carboxymethyl; and imidazolylmethyl;
R⁴ is selected from indolylmethyl and phenylmethyl, and wherein the phenyl part of the phenylmethyl is optionally substituted with one, two, or three groups independently selected from chloro, methyl, methoxy, and trifluoromethyl;
R⁵ is selected from C₃-C₄alkyl; biphenylmethyl, hydroxypropyl; hydroxyisopropyl; and phenymethyl; and wherein the distal phenyl of the biphenylmethyl and the phenyl part of the phenylmethyl are optionally substituted with one, two, or three groups independently selected from aminocarbonyl, carboxy, carboxymethoxy, fluoro, hydroxy, and methylcarbonylamino;
R⁶ is biphenylmethyl;
R⁷ is selected from C₃-C₄alkyl; aminocarbonylC₁-C₂alkyl; aminopropyl; carboxyethyl; hydroxyC₂-C₃alkyl; imidazolylmethyl; methylcarbonylaminobutyl; phenylmethyl; and NH₂C(X)NHpropyl, where X is O or NH; and wherein the phenyl part of the phenylmethyl is optionally substituted with one, two, or three groups independently selected from aminocarbonyl, aminomethyl, carboxy, carboxymethoxy, and hydroxy;
R⁸ is selected from C₁-C₄alkyl; hydroxymethyl; and phenylmethyl; wherein the phenyl part of the phenylmethyl is optionally substituted with one or two hydroxy groups;
R⁹ is selected from isobutyl and methyl;
R^{9′} is hydrogen;
R¹⁰ is selected from aminoC₁-C₄alkyl; aminocarbonylmethyl; carboxymethyl; methyl; methylcarbonylaminoethyl; and NH₂C(X)NHpropyl, where X is O or NH;
R¹¹ is selected from cyclohexylmethyl and isobutyl;
R¹² is selected from C₃-C₄alkyl; aminoC₃-C₄alkyl; hydroxyC₂-C₃alkyl; and phenylmethyl;
R¹³ is selected from aminopropyl; aminocarbonylC₁-C₂alkyl; carboxyethyl; hydroxyC₁-C₂alkyl; imidazolylmethyl; methylcarbonylaminobutyl; and NH₂C(X)NHpropyl, where X is O or NH;
R¹⁴ is aminocarbonyl or —C(O)NR^{14′}CR¹⁵R^{15′}R^{15″}, wherein
R^{14′} is hydrogen;
R¹⁵ is selected from hydrogen; aminoC₁-C₃alkyl; aminocarbonylmethyl;
butylcarbonylaminoethyl; carboxy; carboxyC₁-C₂alkyl; hydroxymethyl; methyl; and methylcarbonylaminoethyl;
R^{15′} is hydrogen or R¹⁵ and R^{15′}, together with the atoms to which they are attached, form a cyclopropyl ring; and
R^{15″} is hydrogen; aminocarbonyl; carboxy; or —(CH₂)ₙC(O)NHCHR¹⁶R^{16′}; wherein
n is 0 or 1;
R¹⁶ is selected from hydrogen; C₃-C₄alkynyl; and aminoC₁-C₄alkyl; and
R^{16′} is hydrogen; C₁-C₂alkyl; aminocarbonyl; or carboxy.

22. The compound of claim 1, or the pharmaceutically acceptable salt thereof, wherein
R¹ is selected from aminocarbonylmethyl; aminoethyl; aminomethyl; aminopropyl; cyclohexylmethyl; 1-hydroxyethyl; imidazolylmethyl; morpholinylmethyl; phenylmethyl; pyridylmethyl; and thienylmethyl; wherein the phenyl part of the phenylmethyl is optionally substituted with a carboxymethoxy, methyl, halo, or trifluoromethyl group;
R² is selected from biphenylmethyl, phenylmethyl, and pyridylmethyl; wherein the distal phenyl of the biphenylmethyl, and the phenyl part of the phenylmethyl are optionally substituted with carboxy, carboxymethoxy, or hydroxy;
R³ is carboxymethyl;
R⁴ is selected from indolylmethyl and phenylmethyl, wherein the phenyl part of the phenylmethyl is optionally substituted with a methyl or a trifluoromethyl group;
R⁵ is selected from C₃-C₄alkyl, biphenylmethyl, and phenymethyl, and wherein distal phenyl of the biphenylmethyl and the phenyl part of the phenylmethyl are optionally substituted with aminocarbonyl, carboxy, carboxymethoxy, methylcarbonylamino, or fluoro;
R⁶ is biphenylmethyl;
R⁷ is selected from C₃-C₄alkyl; aminocarbonylethyl; phenylmethyl; and NH₂C(X)NHpropyl, where X is O or NH; and wherein the phenyl part of the phenylmethyl is optionally substituted with one or two groups independently selected from aminocarbonyl, carboxy, carboxymethoxy, and hydroxy;
R⁸ is methyl;
R⁹ is selected from methyl and butyl;
R^{9′} is hydrogen;
R¹⁰ is selected from aminocarbonylmethyl and aminoethyl;
R¹¹ is selected from butyl and cyclohexylmethyl;
R¹² is selected from hydroxypropyl and propyl;
R¹³ is selected from aminopropyl; carboxyethyl; hydroxyC₁-C₂alkyl; imidazolylmethyl; and methylcarbonylaminobutyl;
R¹⁴ is aminocarbonyl or —C(O)NR^{14′}CR¹⁵R^{15′}R^{15″}, wherein
R^{14′} is hydrogen;
R¹⁵ is selected from hydrogen; aminoC₁-C₂alkyl; aminocarbonylmethyl; and methyl;
R^{15′} is hydrogen; and
R^{15″} is hydrogen; aminocarbonyl; carboxy; or C(O)NHCHR¹⁶R^{16′}; wherein
R¹⁶ is hydrogen; and
R^{16′} is hydrogen or ethyl.

23. A pharmaceutical composition comprising a compound of any one of claims 1 to 22, or a pharmaceutically acceptable salt thereof.

24. A method of enhancing, stimulating, and/or increasing an immune response in a subject in need thereof, wherein the method comprises administering to the subject a therapeutically effective amount of a compound of any one of claims 1 to 22, or a pharmaceutically acceptable salt thereof.

25. A method of blocking the interaction of PD-1 with PD-L1 in a subject, wherein the method comprises administering to the subject a therapeutically effective amount of a compound of any one of claims 1 to 22 or a pharmaceutically acceptable salt thereof.

26. A compound of Formula (II):
or a pharmaceutically acceptable salt thereof, wherein:
R¹ is selected from C₁-C₆alkyl; mono-, di- or tri-C₁-C₆alkylaminoC₁-C₆alkyl; aminoC₁-C₆alkyl; aminocarbonylC₁-C₆alkyl; arylC₁-C₆alkyl; carbamidylC₁-C₆alkyl; cyanoC₁-C₆alkyl;
C₃-C₆cycloalkylcarbonylaminoC₁-C₆alkyl; guanidinylC₁-C₆alkyl; heteroarylC₁-C₆alkyl;
heterocyclylC₁-C₆alkyl; hydroxyC₁-C₆alkyl; and H₂NC(X) , where X is O or NH, and represents an azetidine, pyrrolidine, or piperidine ring; and wherein the aryl part of the arylC₁-C₆alkyl and the heteroaryl part of the heteroarylC₁-C₆alkyl a optionally substituted with one, two, three, four, or five groups independently selected from aminoC₂-C₆alkoxy, aminoC₁-C₆alkyl, aminocarbonyl, carboxy, carboxyC₁-C₆alkoxy halo, hydroxy, and nitro;
R² is selected from C₂-C₆alkenyl; C₁-C₆alkylcarbonylaminoC₁-C₆alkyl-thio-C₁-C₆alkyl; aminocarbonyl C₁-C₆alkyl; arylC₁-C₆alkyl; heteroarylC₁-C₆alkyl; and hydroxyC₁-C₆alkyl; wherein the aryl part of the arylC₁-C₆alkyl is optionally substituted with one, two, three, four, or five groups independently selected from C₂-C₆alkenyl, C₂-C₆alkenyloxy, C₁-C₆alkoxy, C₁-C₆alkyl, C₁-C₆alkylcarbonyloxyC₁-C₆alkoxy, C₂-C₆alkynyloxy, amino, aminoC₁-C₆alkoxy, aminoC₁-C₆alkyl, aminocarbonyl, aryloxy, carboxy, carboxyC₁-C₆alkoxy, cyano, halo, hydroxy, carboxyaryl, nitro, trifluoromethyl, and —OP(O)X¹X², wherein each of X¹ and X² is —OH, —NH₂, or —N(C₁-C₆alkyl)₂;
R³ is selected from aminocarbonylC₁-C₃alkyl; carboxyC₁-C₃alkyl; (OH)₂P(O)OC₁-C₃alkyl; and tetrazolylC₁-C₃alkyl;
R⁴ is selected from arylC₁-C₆alkyl and heteroarylC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, cyano, fluoroC₁-C₆alkyl, and halo;
R⁵ is selected from C₂-C₆alkenyl; C₁-C₆alkyl; C₁-C₆alkylcarbonylaminoC₁-C₆alkylthioC₁-C₆alkyl; aminoC₁-C₆alkyl; aminocarbonylC₁-C₆alkyl; arylC₁-C₆alkyl; carboxyC₁-C₆alkyl; cyanoC₁-C₆alkyl; C₃-C₈cycloalkyl; (C₃-C₈cycloalkyl)C₁-C₆alkyl; and heteroarylC₁-C₆alkyl; and hydroxyC₁-C₆alkyl; wherein the aryl part of the arylC₁-C₆alkyl and the heteroaryl part of the heteroarylC₁-C₆alkyl are optionally substituted with one, two, three, four, or five groups independently selected from C₁-C₆alkyl, fluoroC₁-C₆alkyl, carboxy, aminoC₁-C₆alkyl, aminocarbonyl, carboxyC₁-C₆alkoxy halo, and hydroxy;
R⁶ is aryl-arylC₁-C₃alkyl, heteroaryl-arylC₁-C₃alkyl, aryl-heteroarylC₁-C₃alkyl, heteroaryl-heteroarylC₁-C₃alkyl, wherein the aryl or the heteroaryl part is optionally substituted with one, two, three, four, or five groups independently selected from C₁-C₆alkylcarbonylamino, aminocarbonyl, fluoroC₁-C₆alkyl, halo, hydroxy, trifluoromethoxy, C₁-C₆alkoxy, C₁-C₆alkoxyC₁-C₆alkyl, carboxyC₁-C₆alkoxyC₁-C₆alkyl, cyanoC₁-C₆alkyl, and arylC₁-C₆alkoxy;
R⁷ is selected from hydrogen; C₂-C₆alkenyl; C₁-C₆alkyl; aminoC₁-C₆alkyl; aminocarbonylC₁-C₆alkyl; carboxyC₁-C₆alkyl; arylC₁-C₆alkyl; C₁-C₆alkylcarbonylaminoC₁-C₆alkylthioC₁-C₆alkyl; C₁-C₆alkylcarbonylaminoC₁-C₆alkyl; heteroarylC₁-C₆alkyl; hydroxyC₁-C₆alkyl; and NH₂C(X)NHC₁-C₆alkyl, where X is O or NH; and wherein the aryl part of the arylC₁-C₆alkyl and the heteroaryl part of the heteroarylC₁-C₆alkyl are optionally substituted with one, two, three, four, or five groups independently selected from aminoC₁-C₆alkyl, aminocarbonyl, carboxy, carboxyC₁-C₆alkoxy, and hydroxy;
R⁸ is selected from C₁-C₆alkyl; aminoC₁-C₆alkyl; carboxyC₁-C₆alkyl; aryl; arylC₁-C₆alkyl; heteroarylC₁-C₆alkyl; and hydroxyC₁-C₆alkyl; wherein the aryl part of the arylC₁-C₆alkyl is optionally substituted with one, two, three, four, or five groups independently selected from halo and hydroxy;
R⁹ is selected from hydrogen; C₁-C₆alkyl; aminoC₁-C₆alkyl; aminocarbonylC₁-C₆alkyl; aryl; arylC₁-C₆alkyl; carboxyC₁-C₆alkyl; C₃-C₈cycloalkyl; C₃-C₈cycloalkylC₁-C₆alkyl; heteroarylC₁-C₆alkyl; hydroxyC₁-C₆alkyl; C₁-C₆alkylthioC₁-C₆alkyl; and NH₂C(X)NHC₁-C₆alkyl, where X is O or NH; and wherein the aryl part of the arylC₁-C₆alkyl and the heteroaryl part of the heteroarylC₁-C₆alkyl are optionally substituted with one, two, three, four, or five groups independently selected from C₁-C₆alkoxy, C₁-C₆alkyl, amino, carboxyC₁-C₆alkyl, cyano, halo, hydroxy, nitro, and trifluoromethyl;
R¹⁰ is selected from C₁-C₆alkyl; C₁-C₆alkylcarbonylaminoC₁-C₆alkyl; C₁-C₆alkylNHC₁-C₆alkyl; aminoC₁-C₆alkyl; aminocarbonylC₁-C₆alkyl; carboxyC₁-C₆alkyl; hydroxyC₁-C₆alkyl; NH₂C(X)NHC₁-C₆alkyl, where X is O or NH; heteroarylC₁-C₆alkyl; and arylC₁-C₆alkyl; and wherein the aryl part of the arylC₁-C₆alkyl is optionally substituted with one, two, three, four, or five aminoC₁-C₆alkyl groups;
R¹¹ is selected from C₁-C₆alkyl, arylC₁-C₆alkyl, and C₃-C₈cycloalkylC₁-C₆alkyl; wherein the aryl part of the arylC₁-C₆alkyl is optionally substituted with one, two, three, four, or five groups independently selected from C₁-C₆alkyl, halo, and hydroxy;
R¹² is selected from C₂-C₆alkenyl; C₁-C₆alkyl; C₁-C₆alkylcarbonylaminoC₁-C₆alkylthioC₁-C₆alkyl; aminoC₁-C₆alkyl; arylC₁-C₆alkyl; carboxyC₁-C₆alkyl; hydroxyC₁-C₆alkyl; and NH₂C(X)NHC₁-C₆alkyl, where X is O or NH;
R¹³ is selected from C₂-C₆alkenyl; C₁-C₆alkyl; C₁-C₆alkylcarbonylaminoC₁-C₆alkyl; C₁-C₆alkylcarbonylaminoC₁-C₆alkylthioC₁-C₆alkyl; aminoC₁-C₆alkyl; aminocarbonylC₁-C₆alkyl; carboxyC₁-C₆alkyl; cyanoC₁-C₆alkyl; C₃-C₈cycloalkyl; heteroarylC₁-C₆alkyl; hydroxyC₁-C₆alkyl; and NH₂C(X)NHC₁-C₆alkyl, where X is O or NH;
R¹⁴ is aminocarbonyl or —C(O)NR^{14′}CR¹⁵R^{15′}R^{15″}, wherein
R^{14′} is hydrogen, or R¹⁵ and R^{14′}, together with the atoms to which they are attached, form an azetidine, morpholine, piperidine, piperazine, or pyrrolidine ring, wherein each ring is optionally substituted with an amino or a hydroxy group;
R¹⁵ is selected from hydrogen; C₂-C₆alkenyl; C₁-C₆alkyl; C₁-C₆alkylcarbonylaminoC₁-C₆alkyl; C₁-C₆alkylcarbonylaminoC₁-C₆alkylthioC₁-C₆alkyl; aminoC₁-C₆alkyl; aminocarbonylC₁-C₆alkyl; carboxy; carboxyC₁-C₆alkyl; heterocyclyl; hydroxyC₁-C₆ alkyl; and NH₂C(X)NHC₁-C₆alkyl, where X is O or NH;
R^{15′} is hydrogen, or R¹⁵ and R^{15′}, together with the atoms to which they are attached, form a C₃-C₈cycloalkyl ring; and
R^{15″} is hydrogen; —C(O)NH₂, or —(CH₂)ₙC(O)NHCHR¹⁶R^{16′}; wherein
n is 0, 1, or 2;
R¹⁶ is selected from hydrogen, C₂-C₆alkynyl, aminoC₁-C₆alkyl, and carboxyC₁-C₆alkyl;
R^{16′} is hydrogen; C₁-C₆alkyl; aminocarbonyl; carboxy; or —C(O)NHCHR¹⁷R¹⁷; wherein
R¹⁷ is hydrogen; and
R¹⁷ is —C(O)NHCHR¹⁸R^{18′}; wherein
R¹⁸ is aminoC₁-C₆alkyl; and
R^{18′} is carboxy.
